SEQUENCE LISTING (243 sequences)

SEQ ID NO: 1 - DNA, 35 bp, Rattus rattus
aaaaggtacctctgtgtttttccgttctagtccag

SEQ ID NO: 2 - DNA, 35 bp, Rattus rattus
aaaaccgcggctgaagtatacgtggctctcttgga

SEQ ID NO: 3 - DNA, 34 bp, Rattus rattus
gtgtagcggccgcgacaaggccaatggctatcac

SEQ ID NO: 4 - DNA, 34 bp, Rattus rattus
gtgtagtttaaacgacaaggccaatggctatcac

SEQ ID NO: 5 - DNA, 36 bp, Rattus rattus
ttgtcgcggccgctacacggcagatttgaagacctc

SEQ ID NO: 6 - DNA, 36 bp, Rattus rattus
ttgtcgtttaaactacacggcagatttgaagacctc

SEQ ID NO: 7 - DNA, 39 bp, Rattus rattus
aaaaggtacctcagactggtccagattttagamaagggg

SEQ ID NO: 8 - DNA, 36 bp, Rattus rattus
aaaaccgcggataaatctactggttcgccaagctag

SEQ ID NO: 9 - DNA, 32 bp, Rattus rattus
aaaaggtaccgaggtagtaggaaatgcacttc

SEQ ID NO: 10 - DNA, 33 bp, Rattus rattus
aaaaccgcgggaagagaattattgctgacagtc

SEQ ID NO: 11 - DNA, 22 bp, Rattus rattus
gagcctatcaacgtagatgagg

SEQ ID NO: 12 - DNA, 24 bp, Rattus rattus
cttacatccttcacaggtcatgac

SEQ ID NO: 13 - DNA, 30 bp, Rattus rattus
aaaaggtaccgggagtggatgaaggagttg

SEQ ID NO: 14 - DNA, 28 bp, Rattus rattus
aaaaccgcggcggatcacaagcaataat

SEQ ID NO: 15 - DNA, 33 bp, Rattus rattus
cttcgcggccgcgatctgcaactggagtctttc

SEQ ID NO: 16 - DNA, 33 bp, Rattus rattus
cttcgtttaaacgatctgcaactggagtctttc

SEQ ID NO: 17 - DNA, 32 bp, Rattus rattus
gatcgcggccgcgaagaagggggaagggaatc

SEQ ID NO: 18 - DNA, 32 bp, Rattus rattus
gatcgtttaaacgaagaagggggaagggaatc

SEQ ID NO: 19 - DNA, 30 bp, Rattus rattus
aaaaggtaccgcgtgtgaaaacacaaatgg

SEQ ID NO: 20 - DNA, 30 bp, Rattus rattus
aaaaccgcggaaggaaagaggcattcatgg

SEQ ID NO: 21 - DNA, 30 bp, Rattus rattus
aaaaggtaccattatggaggggaggactgg

SEQ ID NO: 22 - DNA, 29 bp, Rattus rattus
aaaaccgcggacatgtggcaaacaggaga

SEQ ID NO: 23 - DNA, 19 bp, Rattus rattus
tgtcttctgaggaccgccc

SEQ ID NO: 24 - DNA, 19 bp, Rattus rattus
ctgcccagaagactcccgc

SEQ ID NO: 25 - DNA, 30 bp, Mus musculus
aaaaggraccaacaacactaggctcaggag

SEQ ID NO: 26 - DNA, 31 bp, Mus musculus
aaaaccgcggcacatggctaagcacagcatg

SEQ ID NO: 27 - DNA, 31 bp, Mus musculus
cctgcggccgcggactgtcagctggtatttg

SEQ ID NO: 28 - DNA, 31 bp, Mus musculus
cctgtttaaacggactgtcagctggtatttg

SEQ ID NO: 29 - DNA, 31 bp, Mus musculus
gtccgcggccgcagggctgatggccaaaatc

SEQ ID NO: 30 - DNA, 31 bp, Mus musculus
gtccgtttaaacagggctgatggccaaaatc

SEQ ID NO: 31 - DNA, 29 bp, Mus musculus
aaaaggtaccatgctgtgaagcagatacc

SEQ ID NO: 32 - DNA, 34 bp, Mus musculus
aaaaccgcggctgaaaactgaatgagacatttgc

SEQ ID NO: 33 - DNA, 33 bp, Mus musculus
aaaaggtaccgtaatgttccaattgcatcttcc

SEQ ID NO: 34 - DNA, 30 bp, Mus musculus
aaaaccgcggctctcagttctctgctgttg

SEQ ID NO: 35 - DNA, 20 bp, Mus musculus
gatttacccgtggctggaag

SEQ ID NO: 36 - DNA, 20 bp, Mus musculus
ctggactcatggacttcacc

SEQ ID NO: 37 - DNA, 31 bp, Rattus rattus
aaaaggtacctggctcaggagaaaaattgtg

SEQ ID NO: 38 - DNA, 30 bp, Rattus rattus
aaaaccgcggcacggctaaagacagcatga

SEQ ID NO: 39 - DNA, 32 bp, Rattus rattus
ccctgcggccgcggactgtcagctggtatttg

SEQ ID NO: 40 - DNA, 32 bp, Rattus rattus
ccctgtttaaacggactgtcagctggtatttg

SEQ ID NO: 41 - DNA, 31 bp, Rattus rattus; identical to SEQ ID NO: 29

SEQ ID NO: 42 - DNA, 31 bp, Rattus rattus; identical to SEQ ID NO: 30

SEQ ID NO: 43 - DNA, 30 bp, Rattus rattus
aaaaggtaccggagataggctggtttgacg

SEQ ID NO: 44 - DNA, 29 bp, Rattus rattus
aaaaccgcggatggtggtagttcggatgg

SEQ ID NO: 45 - DNA, 31 bp, Rattus rattus
aaaaggtaccaggttgttcttggagatgtgc

SEQ ID NO: 46 - DNA, 30 bp, Rattus rattus
aaaaccgcggtcctcttggctggtgagttt

SEQ ID NO: 47 - DNA, 20 bp, Rattus rattus
gatttactcgcggctggaag

SEQ ID NO: 48 - DNA, 19 bp, Rattus rattus
ctggactcacgggcttcac

SEQ ID NO: 49 - DNA, 30 bp, Homo sapiens
aaagcggccgcttggggttgcgccttttcc

SEQ ID NO: 50 - DNA, 30 bp, Homo sapiens
aaaagcggccgccatagagcccaccgcatc

SEQ ID NO: 51 - DNA, 192 bp, Felis catus
  misc_feature (150)..(150): n is a, c, g, or t
catgactagtccatccaagacccacatacatctcctccctccacagcccatgactagtccatccaagacccacatacatctcctccctccacagcccatgactagtccatccaagacccacatacatctcctccctccacagcccatgantagtccatccaagacccacatacatctcntccctncacagcc

SEQ ID NO: 52 - DNA, 372 bp, Felis catus
gcacgcgcaacgtgaaggttggggaggcggggaaggaaaggaaattaccatgactagtccatccaagaccctggataagagtccgttctccacgggaaggggagccaaggtagcgtcatctgttgactcggggacttaggatcctgcccacacatacatctcctccctccacagcccccaggcagtaagcagcccagagaggcctggcggtgcctcctggaaaaggatgttagacgcagccctcccaccctgccctactgttgcagccacagcaggtatgttccaggctggggagggagcacctgccactgcatcatgaagggggctcgtgttctcgtgcttctctgggctgccttgctcttgatctggggt

SEQ ID NO: 53 - protein, 149 aa, Artificial Sequence
  SYNTHESIZED
VPAAMAERPFQCRICMRNFSRSDNLARHIRTHTGEKPFACDICGRKFAQSSDLRRHTKIHTGGQRPFQCRICMRNFSRSDTLSQHIRTHTGEKPFACDICGRKFADRSARTRHTKIHTGEKPFQCRICMRKFAQSSDLRRHTKIHLRGS

SEQ ID NO: 54 - protein, 181 aa, Artificial Sequence
  SYNTHESIZED
VPAAMAERPFQCRICMRNFSERGTLARHIRTHTGEKPFACDICGRKFAQSADRTKHTKIHTGGQRPFQCRICMRNFSRSDHLSTHIRTHTGEKPFACDICGRKFADNANRTKHTKIHTGGGGSQKPFQCRICMRNFSQSSNLARHIRTHTGEKPFACDICGRKFARSDALTQHTKIHLRGS

SEQ ID NO: 55 - DNA, 28 bp, Felis catus
ctgctgtcctggctgaggccctgatgct

SEQ ID NO: 56 - DNA, 28 bp, Felis catus
gaatggatttactggtcagccagctact

SEQ ID NO: 57 - protein, 149 aa, Artificial Sequence
  SYNTHESIZED
VPAAMAERPFQCRICMRNFSRSDHLSTHIRTHTGEKPFACDICGRKFARSAHLSRHTKIHTGSQKPFQCRICMRNFSQSGSLTRHIRTHTGEKPFACDICGRKFARSDHLTQHTKIHTGEKPFQCRICMRKFALKQHLNEHTKIHLRGS

SEQ ID NO: 58 - protein, 178 aa, Artificial Sequence
  SYNTHESIZED
VPAAMAERPFQCRICMRNFSRSDNLSAHIRTHTGEKPFACDICGRKFAQSANRIKHTKIHTGSQKPFQCRICMRNFSQSGALARHIRTHTGEKPFACDICGRKFARSDNLREHTKIHTGSQKPFQCRICMRNFSRSDHLSEHIRTHTGEKPFACDICGRKFAQSATRKKHTKIHLRGS

SEQ ID NO: 59 - protein, 186 aa, Artificial Sequence
  SYNTHESIZED
VPAAMAERPFQCRICMRNFSQSGHLARHIRTHTGEKPFACDICGRKFAQSADRTKHTKIHTGSQKPFQCRICMRNFSRSDTLSEHIRTHTGEKPFACDICGRKFANRRGRWSHTKIHTPNPHRRTDPSHKPFQCRICMRNFSRSDHLSRHIRTHTGEKPFACDICGRKFADPSYLPRHTKIHLRGS

SEQ ID NO: 60 - protein, 121 aa, Artificial Sequence
  SYNTHESIZED
VPAAMAERPFQCRICMRNFSRSDSLSVHIRTHTGEKPFACDICGRKFAQNANRKTHTKIHTGSQKPFQCRICMRNFSRSANLARHIRTHTGEKPFACDICGRKFATSGSLTRHTKIHLRGS

SEQ ID NO: 61 - protein, 153 aa, Artificial Sequence
  SYNTHESIZED
VPAAMAERPFQCRICMRNFSRSDTLSAHIRTHTGEKPFACDICGRKFADKRTRTTHTKIHTHPRAPIPKPFQCRICMRNFSTSGSLSRHIRTHTGEKPFACDICGRKFADSSDRKKHTKIHTGEKPFQCRICMRKFARSDNLTRHTKIHLRGS

SEQ ID NO: 62 - protein, 153 aa, Artificial Sequence; identical to SEQ ID NO: 61
  SYNTHESIZED

SEQ ID NO: 63 - DNA, 28 bp, Felis catus
acagtagggcagggtgggagggctgcgt

SEQ ID NO: 64 - DNA, 28 bp, Felis catus
ggccacagcaggtataaaagggttccag

SEQ ID NO: 65 - DNA, 28 bp, Felis catus
tcgtcgggggttcccgtcaggaataggt

SEQ ID NO: 66 - DNA, 28 bp, Felis catus
atgttgagcaagtggcacaatacaatgc

SEQ ID NO: 67 - DNA, 28 bp, Felis catus
aagagtccgttctccacgtagcaatcct

SEQ ID NO: 68 - DNA, 28 bp, Felis catus
ccagggtcttggatggactagtcatggt

SEQ ID NO: 69 - protein, 42 aa, Artificial Sequence
  SYNTHESIZED
QSGNLARLAYDRRKRSDTLSEQSSHLARQSSDLSRRRDTLLD

SEQ ID NO: 70 - protein, 35 aa, Artificial Sequence
  SYNTHESIZED
QSGDLTRNKHHRNRRSDALARTSGNLTRRRYYLRL

SEQ ID NO: 71 - protein, 35 aa, Artificial Sequence
  SYNTHESIZED
RSDNLARWRGDRVKDRSHLARQSSDLSRQSGDLTR

SEQ ID NO: 72 - protein, 42 aa, Artificial Sequence
  SYNTHESIZED
RSDNLSESSRNLASRSANLARRSDNLTRRSDNLSESSRNLAS

SEQ ID NO: 73 - protein, 35 aa, Artificial Sequence
  SYNTHESIZED
DSSDRKKQSSDLSRYHWYLKKRSDHLSQTSANRTT

SEQ ID NO: 74 - protein, 42 aa, Artificial Sequence
  SYNTHESIZED
QSGNLARWLSSLGIDRSDLSRLRFNLRNQSGDLTRQSGNLAR

SEQ ID NO: 75 - protein, 35 aa, Artificial Sequence
  SYNTHESIZED
DRSNLSRDAFTRTRRSDNLSVERGTLARQSGDLTR

SEQ ID NO: 76 - protein, 35 aa, Artificial Sequence
  SYNTHESIZED
TNHGLNETSSNLSRQSSDLSRHKYHLRSQSGHLSR

SEQ ID NO: 77 - DNA, 28 bp, Felis catus
atccggctggaccgtctgaactcctagc

SEQ ID NO: 78 - DNA, 28 bp, Felis catus
agtgggatgtgggtgcacccaggccgga

SEQ ID NO: 79 - DNA, 28 bp, Felis catus
cagcagctggccctgaggggacacacag

SEQ ID NO: 80 - DNA, 28 bp, Felis catus
tgcaccagtgaggagcaccaggctggga

SEQ ID NO: 81 - DNA, 28 bp, Felis catus
tacagtggtttgcttcccccggacccat

SEQ ID NO: 82 - DNA, 28 bp, Felis catus
gggaagcaccatgcctgtgaacatgttc

SEQ ID NO: 83 - DNA, 28 bp, Felis catus
aggcagccaaggcggacccatcgaagga

SEQ ID NO: 84 - DNA, 28 bp, Felis catus
gaggacgtgctgatcgtgactacccagt

SEQ ID NO: 85 - DNA, 46 bp, Homo sapiens
tgtcgccttcgcctcctaatccctagccactatggtgagtaagccg

SEQ ID NO: 86 - DNA, 15 bp, Homo sapiens
cttcgcctcctaatc

SEQ ID NO: 87 - DNA, 15 bp, Homo sapiens
cactatggtgagtaa

SEQ ID NO: 88 - DNA, 18 bp, Homo sapiens
gtcgtcgacaacggctcc

SEQ ID NO: 89 - DNA, 19 bp, Homo sapiens
tgcaaggccggcttcgcgg

SEQ ID NO: 90 - DNA, 15 bp, Homo sapiens
cctcgccgccccgct

SEQ ID NO: 91 - DNA, 16 bp, Homo sapiens
gccgcccgccatggcg

SEQ ID NO: 92 - DNA, 300 bp, Rattus rattus
gggagactgaaacctctgatatcttctttcaggccgtgaggaattctacagcactcacccccacttcatgacccagagagccctgtacctggctgtctacgacctcagcaaggggcaggcggaggtggatgccatgaagccctggctcttcaacatcaaggtgatctcttctggtgccatggaaacagagtgggaccgttgctcactgttgtatgctatgcttctggttttgttttgttttgctttgtttgagtccgaatcttactatgtatccctggctgccctggaactttctgtgaa

SEQ ID NO: 93 - DNA, 200 bp, Rattus rattus
tcttctttcaggccgtgaggaattctacagcactcacccccacttcatgacccagagagccctgtacctggctgtctacgacctcagcaaggggcaggcggaggtggatgccatgaagccctggctcttcaacatcaaggtgatctcttctggtgccatggaaacagagtgggaccgttgctcactgttgtatgctatgc

SEQ ID NO: 94 - DNA, 28 bp, Rattus rattus
tgggtcatgaagtgggggtgagtgctgt

SEQ ID NO: 95 - DNA, 28 bp, Rattus rattus
gagccctgtacctggctgtctacgacct

SEQ ID NO: 96 - DNA, 28 bp, Rattus rattus
agtcagcacaggcatgtccatgttgagt

SEQ ID NO: 97 - DNA, 28 bp, Rattus rattus
cctctggggtagtgaacaggtctcccac

SEQ ID NO: 98 - DNA, 28 bp, Rattus rattus
aagccgactagagagagaacccaaacgc

SEQ ID NO: 99 - DNA, 28 bp, Rattus rattus
gtgaaggagatcctcaaggagcaggaga

SEQ ID NO: 100 - DNA, 28 bp, Rattus rattus
gaactcggagtttcccaggctggacctt

SEQ ID NO: 101 - DNA, 28 bp, Rattus rattus
gtgcggcacctgcagacaagcaaccctc

SEQ ID NO: 102 - DNA, 28 bp, Rattus rattus
gggtagtagtgtgggggtagcatgtcag

SEQ ID NO: 103 - DNA, 28 bp, Rattus rattus
aaggcctgggccacggccgcacactctt

SEQ ID NO: 104 - protein, 35 aa, Artificial Sequence
  SYNTHESIZED
WRSCRSAQSGSLTRRSDNLREQSGSLTRQSADRTK

SEQ ID NO: 105 - protein, 42 aa, Artificial Sequence
  SYNTHESIZED
RSDHLSADRSNRKTRSAALSRQSGSLTRRSDHLSERKHDRTK

SEQ ID NO: 106 - protein, 35 aa, Artificial Sequence
  SYNTHESIZED
RSDALSVQSQHRTTRSDNLSVDRSNLTRDRSDLSR

SEQ ID NO: 107 - protein, 35 aa, Artificial Sequence
  SYNTHESIZED
RSDNLSTDNSSRITTSSNLSRQSGHLQRQSGNLAR

SEQ ID NO: 108 - protein, 42 aa, Artificial Sequence
  SYNTHESIZED
ASTGLIRRSDHLSRRSDALSRQSGNLARNNTQLIETSSILSR

SEQ ID NO: 109 - protein, 42 aa, Artificial Sequence
  SYNTHESIZED
DRSALSRQSSDLRRRSDVLSADRSNRIKRSDSLSADRSSRTK

SEQ ID NO: 110 - protein, 35 aa, Artificial Sequence
  SYNTHESIZED
RSDNLSQASNDRKKHRSSLRRRSDHLSEARSTRTN

SEQ ID NO: 111 - protein, 35 aa, Artificial Sequence
  SYNTHESIZED
DRSNLSRQSGDLTRHKTSLKDQSGDLTRRSDDLTR

SEQ ID NO: 112 - protein, 35 aa, Artificial Sequence
  SYNTHESIZED
RSSHLSRRSDHLSTASSARKTQSGALARQSGSLTR

SEQ ID NO: 113 - protein, 42 aa, Artificial Sequence
  SYNTHESIZED
QSGDLTRDRSDLSRRSDTLSVDNSTRIKRSDALSVDSSHRTR

SEQ ID NO: 114 - DNA, 300 bp, Rattus rattus
caagagcactgtaggtcctgacccagccttaaacttactactctacacaggatgcctggccgagggagagctggaggtgacagatcagctcccagggcaaagcgaccaaccctgggagcaggccctgaaccgcttctgggattacctgcgctgggtgcagacgctttctgaccaggtccaggaagagctgcagagctcccaagtcacacaggaactgacgtgagtgctcagcgcttcaccctccgcacctgctgagtatccagatccaggggttcctcctatctgggcacctacctactt

SEQ ID NO: 115 - DNA, 400 bp, Rattus rattus
caagagcactgtaggtcctgacccagccttaaacttactactctacacaggatgcctggccgagggagagctggaggtgacagatcagctcccagggcaaagcgaccaaccctgggagcaggccctgaaccgcttctgcgattacctgcgctgggtgcagacgctttctgaccaggtccaggaagagctgcagagctcccaagtcacacaggaactgacgtgagtgctcagcgcttcaccctccgcacctgctgagtatccagatccaggggttcctcctatctgggcacctacctacttgtttcctttctccatgagtgtgtgggccaggttggccttgaactctcaatacttctgctttctttagccttctggatactgggatgaacaggcattcatt

SEQ ID NO: 116 - DNA, 300 bp, Rattus rattus
aagaagaagaagaccccagcgaggaaaatgtgctggagacccctgtgccggttcctgtggctttggtcctatctgtcctatgttcaagctgtgcctatccacaaagtccaggatgacaccaaaaccctcatcaagaccattgtcaccaggatcaatgacatttcacacacggtagggagtctcttgtggggaagcaaaggagaagtagggctagaaccagagtctgagaatcacagtgagcgccttctgagtgtcatgcaccccctagaacctgagaatttgtaagcctagggtgtccat

SEQ ID NO: 117 - DNA, 28 bp, Rattus rattus
aagcggttcagggcctgctcccagggtt

SEQ ID NO: 118 - DNA, 28 bp, Rattus rattus
gggattacctgcgctgggtgcagacgct

SEQ ID NO: 119 - DNA, 28 bp, Rattus rattus
gtggataggcacagcttgaacataggac

SEQ ID NO: 120 - DNA, 28 bp, Rattus rattus
aagtccaggatgacaccaaaaccctcat

SEQ ID NO: 121 - DNA, 20 bp, Rattus rattus
ccccagtttgtggtctgcca

SEQ ID NO: 122 - DNA, 18 bp, Rattus rattus
gctaaaggtgaagatcta

SEQ ID NO: 123 - protein, 35 aa, Artificial Sequence
  SYNTHESIZED
RSDALSVDSSHRTRRSDNLSETSGSLTRRSDDLTR

SEQ ID NO: 124 - protein, 35 aa, Artificial Sequence
  SYNTHESIZED
RSDHLSRQSSDLRRRSDVLSADRSNRIKTSSNLSR

SEQ ID NO: 125 - protein, 28 aa, Artificial Sequence
  SYNTHESIZED
RSDALSEQNATRTKRSDYLSTQNAHRKT

SEQ ID NO: 126 - protein, 35 aa, Artificial Sequence
  SYNTHESIZED
DQSTLRNDRSNLSRTSANLSRRSDNLSEDRSALAR

SEQ ID NO: 127 - DNA, 700 bp, Rattus rattus
agtcattgctggaagaatgcctatctgggcaggacatttttaatgctacagtttttaaatgtgctctttagctacatactccatactacatgctacatgctacatgctacattagtgaaacatgctccagccatggtaaaatgtctctgggtgcttctttagttggcactggcatctgctgtgtcctgctcctttacacgattctctgtcctggggaatgattggctctcttacaaaatggagcattcttctcaacttgccttccggtctcctttccagttcccacgacggcagccagcacccctgacgcagtcgacaagtacctggagacccccggagatgagaacgagcacgcccatttccagaaagccaaagagaggttggaagccaagcaccgagagagaatgtcccaggtacggagaaggcttccaacttctgctgttctgttgtctagggagatgcacgctcgcctctgcctcagacgggtagatacaaagtttaatttaaatgttttccacgaggacacagattgtagggttcccctacatctatccagtgtgcgatcacatcaaggaaaggcaagtacaagaggattttgaagcacataatcaattgtgcctctccgctaaagaaaaggtactctgcgagatggtgaggcaagggctgttaactgatacattgtagtaaactttgtgtgtgt

SEQ ID NO: 128 - DNA, 700 bp, Rattus rattus; identical to SEQ ID NO: 127

SEQ ID NO: 129 - DNA, 19 bp, Rattus rattus
gccagcacccctgacgcag

SEQ ID NO: 130 - DNA, 17 bp, Rattus rattus
tcgacaagtacctggag

SEQ ID NO: 131 - DNA, 1000 bp, Rattus rattus
gtccccaaatattatcaggaaaaggattgatcatttatggctgtacgatcggcacctaacagagcttagtaaaatagatttaatagctatctttggaaagaattaaatgaatgaaaatttgtgtgcattgatctcagttatttccagaatactgcaatatgatatgtatacttttcctttttgttttttgtttgtttgacacttttcttaacttgacttttccctccctccattttccccaggtagcttcgccaaaatggctgtccccttgccatctaccctgagactcagttctgcacctgatgaaattcagcacccgcacatcaaattttccgagtggaaatttaagctgtttagggtgagatcctttgaaaaggcacccgaagaagcacaaaaagagaaggattcctcagaagggaaaccttgtctcgagcagtctccagtagttctagataaccctgggggtcagaattcagttctgactcaacgagcattgaaactccatcctaaattttcaaagaaattccatgttgatgggaagtcaagcgacaaagcaattcaccaagccaggcttagacacttctgccgcatctgtggcaatcacttcaagagtgacgggcacaaccggagatacccagtccacgggcccgtggacgctaaaactcaaagccttttccgaaagaaggaaaaaagagtcacgtcctggccagatctcattgccagagttttccggattgatgtgaagtcagatgttgactccatccaccccactgaattctgccataactgttggagcattatgcacaggaagttcggcagtgctcacagtcaggtctactgcccaaggaatgtgaccgtggagtggcacccccacacaccgtcctgtgacatctgctttactgcccatcggggactgaagaggaagagacatcagcccaacgtgcagctcagcaagaaactaaaaactgtgctcaaccatgctagacgggaccgtcgcaag

SEQ ID NO: 132 - DNA, 200 bp, Rattus rattus
tgcacaggaagttcggcagtgctcacagtcaggtctactgcccaaggaatgtgaccgtggagtggcacccccacacaccgtcctgtgacatctgctttactgcccatcggggactgaagaggaagagacatcagcccaacgtgcagctcagcaagaaactaaaaactgtgctcaaccatgctagacgggaccgtcgcaag

SEQ ID NO: 133 - DNA, 300 bp, Rattus rattus
tatcatccacggagggaaaacgccaaacaatgagctttccgataagatttatatcatgtctgtcgcttgcaagaataacaaaaaagttactttccgttgtacagagaaagacttagtaggagatgtccctgaagccagatatggccattccattgacgtggtatatagccgaggaaaaagtgttggtgttctctttggaggacggtcatacatgccttctacccaaagaaccacagaaaaatggaatagtgtagctgattgcctaccccatgttttcttggtagattttgaatttgggtg

SEQ ID NO: 134 - DNA, 300 bp, Rattus rattus; identical to SEQ ID NO: 133

SEQ ID NO: 135 - DNA, 20 bp, Rattus rattus
ccccagtttgtggtctgcca

SEQ ID NO: 136 - DNA, 18 bp, Rattus rattus
gctaaaggtgaagatcta

SEQ ID NO: 137 - DNA, 28 bp, Rattus rattus
ttccttgggcagtagacctgactgtgag

SEQ ID NO: 138 - DNA, 28 bp, Rattus rattus
gtgaccgtggagtggcacccccacacac

SEQ ID NO: 139 - DNA, 28 bp, Rattus rattus
acgtggtatatagccgaggaaaaagtgt

SEQ ID NO: 140 - DNA, 28 bp, Rattus rattus
ataccacgtcaatggaatggccatatct

SEQ ID NO: 141 - DNA, 28 bp, Rattus rattus
ttaagggccatgaagatgaggatgctac

SEQ ID NO: 142 - DNA, 28 bp, Rattus rattus
cagcaagaccggaagccttccagtcagt

SEQ ID NO: 143 - DNA, 28 bp, Rattus rattus
ttgtcgattttggaaggattgagggccc

SEQ ID NO: 144 - DNA, 28 bp, Rattus rattus
atgcaggaagagctgcagaagtggaaga

SEQ ID NO: 145 - DNA, 28 bp, Rattus rattus
tacacaagtccttctccaggagctagaa

SEQ ID NO: 146 - DNA, 28 bp, Rattus rattus
acaaagcttatgaaggtcttagtgaaaa

SEQ ID NO: 147 - protein, 35 aa, Artificial Sequence
  SYNTHESIZED
DRSNLSRQSGSLTRERGTLARRSDHLTTHKTSLKD

SEQ ID NO: 148 - protein, 35 aa, Artificial Sequence
  SYNTHESIZED
QNATRIKRSDALSRQSGHLSRRSADLTEDRANLSR

SEQ ID NO: 149 - protein, 35 aa, Artificial Sequence
  SYNTHESIZED
RSDNLSRDSSTRKKNSGNLDKQSGALARRSDALAR

SEQ ID NO: 150 - protein, 35 aa, Artificial Sequence
  SYNTHESIZED
QSGNLARRSDSLSVQSADRTKRSDTLSTDRKTRIN

SEQ ID NO: 151 - protein, 35 aa, Artificial Sequence
  SYNTHESIZED
TSGNLTRQSGNLARLKQNLDADRSHLTRRLDNRTA

SEQ ID NO: 152 - protein, 35 aa, Artificial Sequence
  SYNTHESIZED
DRSDLSRQSGNLARRSDTLSEQRQHRTTQNATRIK

SEQ ID NO: 153 - protein, 35 aa, Artificial Sequence
  SYNTHESIZED
RSDHLSAQSGHLSRDSESLNATSSNLSRDRSSRKR

SEQ ID NO: 154 - protein, 35 aa, Artificial Sequence
  SYNTHESIZED
QSGSLTRQSSDLRRQRTHLTQQSGHLQRQSGDLTR

SEQ ID NO: 155 - protein, 35 aa, Artificial Sequence
  SYNTHESIZED
QSGDLTRSSSDRKKDSSDRKKRSDNLSTDNSNRIN

SEQ ID NO: 156 - protein, 35 aa, Artificial Sequence
  SYNTHESIZED
TSGHLSRQSGNLARHLGNLKTQSSDLSRQSGNRTT

SEQ ID NO: 157 - DNA, 300 bp, Rattus rattus
attaatgtactcactcttggcaaaacaaaactggctcaggagaaaaattgtgtatttgcaattccgacattatgtgtaacacaatctctttcttcttttagtgacgtctccaaaattaatgaaggaattggtgacaaaattggaatgttctttcaggcaatggcaacattttttggtggttttataataggatttactcgcggctggaagctaactcttgtgattttggccatcagccctgttcttggactgtcagctggtatttgggcaaaggtaggtgaagcccgtgagtccagattt

SEQ ID NO: 158 - DNA, 200 bp, Rattus rattus
gtgacgtctccaaaattaatgaaggaattggtgacaaaattggaatgttctttcaggcaatggcaacattttttggtggttttataataggatttactcgcggctggaagctaactcttgtgattttggccatcagccctgttcttggactgtcagctggtatttgggcaaaggtaggtgaagcccgtgagtccagattt

SEQ ID NO: 159 - DNA, 400 bp, Rattus rattus
ggtaaaaggaacaattgattgacagcacgcttgcttggtagagtctgcctgaagtatacagttcacactagattatgtgcgtggcagggctcttctgtttgctgagtgacttgtcactggctttttcttagaacctgggcccttctgtgctggctggggtggctgttatgatcctcatggtgcccttcaatgctgtgatggccatgaagaccaagacttaccaggtaggatgtccaactccatgagacttcattctgagccctggcctgggtctttccaggtgagggtcgcccagtttcctgatgcttgggcctaggatagacagcctaatctattataggtgtcataatacagggatactgcttccctcagtggccttcaaatgtctgcaagtttgcca

SEQ ID NO: 160 - DNA, 200 bp, Rattus rattus
gctgagtgacttgtcactggctttttcttagaacctgggcccttctgtgctggctggggtggctgttatgatcctcatggtgcccttcaatgctgtgatggccatgaagaccaagacttaccaggtaggatgtccaactccatgagacttcattctgagccctggcctgggtctttccaggtgagggtcgcccagtttcc

SEQ ID NO: 161 - DNA, 900 bp, Rattus rattus
atgcacatactttaaaaatcaactcattaaatttaaatatctaacaacaaggatatcctaactatctatctgataagaatcataatttgaaagcagcaattactgtgagttagctatggaaacatctacggtttctcctggtaacaaagacaccatgttatgctcatattaggagaaaaggctggatctcagttatggattgttacaaataaaggtgaagctagttttccatccacattctccagccctgaattggataggcccgatctagacatagactgtgaagacagtggtgaggtcactccatttcctaagggcatcatggagctccctcgaggacctgtcagagtgtagagaggtggccggtacctgatggagaaggagccataggaggacttttgattcaatagagcctctctccgtctcttctccatcctgtcttctagcatattctcttgtctctgtgctctccccccacgcttctgtcaaataaaggaccaggagatgtagacagtggaacagtagagaagtccaatgcttcatcctctgcctcagatctggaggataggttctgacagaagcagactgacgtagcccagagagactcagcctgtggactgttctcattccaggactgttctgaaaggttacaagcatccactgacactagaagatgtctgggatatcgatgaagggtttaaaacaaggtcagtcaccagcaagtttgaggcggccatgacaaaggacctgcagaaagccaggcaggcttttcagaggcggctgcagaagtcccagcggaaacctgaggccacactacacggactgaacaagaagcagagtcagagccaagacgttctcgtcctggtaactttaactcaagtgtccgtgtgaatgcactgt

SEQ ID NO: 162 - DNA, 900 bp, Rattus rattus
ccaggcccacgtgtggcacacaggcatatgtgcaggcaaaatacccatatacatcaaaaaaaatttttaagaaaaaatagaataattgagcctaaataagtggggagttagtagctatgaatttgaaataggcgttaggccagtttgagagaactgtaaagttgagaccagagctaaagtgtgctgtcctgtctatgttctcttgagaatgacagaagagaattagaatctctccacttagtaactgaagagacaatgatgacgtgcagatcttgaagtcagagagcacaagaacagttaatgttttcccttccttccttccttccttccttccttccttccttccttccttccttctttctttctttctggacttggatttagatctcagacagaagactagtgagcaccagagcatatcagtagtcctccgtggctcataatttatgatatacaccaatgcatgagtttctcaagtgtccaaaactcctctggaattattggagtatttgttaagcatatgagaaaaggatgtgttagtgaagggagagtgagaatggggagaacttggcgcgttctgtcttcccaagccttccctgcctcatctgaatacaccttctctattccaagaccatcagggtcgtgtgtccttgtgttgccttctcttgcaggttaccactgtgagccctacaacaaccctgcagacttcttccttgacgtcatcaacggagattcttcggctgtaatgttaaacagaggggaacaagaccatgaaggtatatgagtcttgtagattcatacgttggtgtccaattgtttgattgaagcccagtagtgtgtcttattaaagcatgcttttgtaggccagccaggactatgtagtgagatcctaccttaaaa

SEQ ID NO: 163 - DNA, 800 bp, Rattus rattus
tcttgagaatgacagaagagaattagaatctctccacttagtaactgaagagacaatgatgacgtgcagatcttgaagtcagagagcacaagaacagttaatgttttcccttccttccttccttccttccttccttccttccttccttccttccttctttctttctttctggacttggatttagatctcagacagaagactagtgagcaccagagcatatcagtagtcctccgtggctcataatttatgatatacaccaatgcatgagtttctcaagtgtccaaaactcctctggaattattggagtatttgttaagcatatgagaaaaggatgtgttagtgaagggagagtgagaatggggagaacttggcgcgttctgtcttcccaagccttccctgcctcatctgaatacaccttctctattccaagaccatcagggtcgtgtgtccttgtgttgccttctcttgcaggttaccactgtgagccctacaacaaccctgcagacttcttccttgacgtcatcaacggagattcttcggctgtaatgttaaacagaggggaacaagaccatgaaggtatatgagtcttgtagattcatacgttggtgtccaattgtttgattgaagcccagtagtgtgtcttattaaagcatgcttttgtaggccagccaggactatgtagtgagatcctaccttaaaacaaaaatcgtgggttggggattttgctcagtggtagagcgcttgcctagaaagcgcaaggccctgggttcagtccccagctccgaaaaaaaagaaccaaa

SEQ ID NO: 164 - DNA, 28 bp, Rattus rattus
acagggctgatggccaaaatcacaagag

SEQ ID NO: 165 - DNA, 28 bp, Rattus rattus
ttggactgtcagctggtatttgggcaaa

SEQ ID NO: 166 - DNA, 28 bp, Rattus rattus
aggaggggaagcagggttccgtggatga

SEQ ID NO: 167 - DNA, 28 bp, Rattus rattus
atgctggtgttcggatacatgacagata

SEQ ID NO: 168 - DNA, 28 bp, Rattus rattus
gaagggcccaggttctaagaaaaagcca

SEQ ID NO: 169 - DNA, 28 bp, Rattus rattus
tgctggctggggtggctgttatgatcct

SEQ ID NO: 170 - DNA, 28 bp, Rattus rattus
ttgctggtgactgaccttgttttaaacc

SEQ ID NO: 171 - DNA, 28 bp, Rattus rattus
ttgaggcggccatgacaaaggacctgca

SEQ ID NO: 172 - DNA, 28 bp, Rattus rattus
atgacgtcaaggaagaagtctgcagggt

SEQ ID NO: 173 - DNA, 28 bp, Rattus rattus
acggagattcttcggctgtaatgttaaa

SEQ ID NO: 174 - DNA, 29 bp, Rattus rattus
ctgtttcttgacaaaacaacactaggctc

SEQ ID NO: 175 - DNA, 25 bp, Rattus rattus
gggtcatgggaaagagtttaaaatc

SEQ ID NO: 176 - DNA, 20 bp, Rattus rattus
catgctgtgaagcagatacc

SEQ ID NO: 177 - DNA, 24 bp, Rattus rattus
ctgaaaactgaatgagacatttgc

SEQ ID NO: 178 - DNA, 22 bp, Rattus rattus
gccgataaaagagccatgtttg

SEQ ID NO: 179 - DNA, 21 bp, Rattus rattus
gataaggagaaaagctgcacc

SEQ ID NO: 180 - DNA, 33 bp, Rattus rattus
gccatcagccctgttcttggactgtcagctggt

SEQ ID NO: 181 - DNA, 33 bp, Rattus rattus
gccatcagctctcaaagaggactgtaagaagct

SEQ ID NO: 182 - DNA, 32 bp, Rattus rattus
gccaacagctctattttggactctccgctgct

SEQ ID NO: 183 - DNA, 33 bp, Rattus rattus
gccatcagctctataacatgactgtctactgat

SEQ ID NO: 184 - DNA, 33 bp, Rattus rattus
gtcaccaaccctctccatggaaagtcagctggt

SEQ ID NO: 185 - DNA, 33 bp, Rattus rattus
gacttcagccctgactgctgactggcaactggt

SEQ ID NO: 186 - DNA, 33 bp, Rattus rattus
gccagcagccctttccttgaagggtcagctagt

SEQ ID NO: 187 - DNA, 33 bp, Rattus rattus
gccatcagcccgctcatgagcctgtttgctggt

SEQ ID NO: 188 - DNA, 32 bp, Rattus rattus
gccagcagccctgcctgggcctggcagttagt

SEQ ID NO: 189 - DNA, 33 bp, Rattus rattus
gctgtcagccctcttattggattgtcatctgct

SEQ ID NO: 190 - DNA, 33 bp, Rattus rattus
gccctcagccctcgagatgctctgtcatcaggt

SEQ ID NO: 191 - DNA, 32 bp, Rattus rattus
gccatcagcccactgtgggactttgagtgggt

SEQ ID NO: 192 - DNA, 32 bp, Rattus rattus
cacctgagcccgcaactggactgtcagctggt

SEQ ID NO: 193 - DNA, 33 bp, Rattus rattus
cccatcaacactaacacaggactggcatctggt

SEQ ID NO: 194 - DNA, 32 bp, Rattus rattus
tccagcagctctgtctgggactgttagatggt

SEQ ID NO: 195 - DNA, 32 bp, Rattus rattus
cccaacagccctattagggacaggcacctggt

SEQ ID NO: 196 - DNA, 32 bp, Rattus rattus
gccatcaggcatggagaggacattcagctgga

SEQ ID NO: 197 - DNA, 32 bp, Rattus rattus
gccatcgcccctggcctggatggtctgctggt

SEQ ID NO: 198 - DNA, 33 bp, Rattus rattus
cccatcagcactgtggacggtcggtcatctggt

SEQ ID NO: 199 - DNA, 33 bp, Rattus rattus
gccaggagcctttcaagtggactgtcagttgct

SEQ ID NO: 200 - DNA, 33 bp, Rattus rattus
gccagcagctgtgactgtgggctatcagctggt

SEQ ID NO: 201 - protein, 28 aa, Artificial Sequence
  SYNTHESIZED
DRSHLSRTSGNLTRQSSDLSRRSDHLTQ

SEQ ID NO: 202 - protein, 35 aa, Artificial Sequence
  SYNTHESIZED
TSGHLSRQSSDLSRQSADRTKRSDVLSEQSGHLSR

SEQ ID NO: 203 - protein, 35 aa, Artificial Sequence
  SYNTHESIZED
TSGHLSRRSDNLSERNANRITRSDHLSERNDNRKR

SEQ ID NO: 204 - protein, 28 aa, Artificial Sequence
  SYNTHESIZED
RSDHLSENNSSRTRTSGHLSRQSSDLRR

SEQ ID NO: 205 - protein, 35 aa, Artificial Sequence
  SYNTHESIZED
TNGQLKETSSSLSRRSDNLSEASKTRKNRSDHLTQ

SEQ ID NO: 206 - protein, 35 aa, Artificial Sequence
  SYNTHESIZED
DRSALSRRSDALARRSDHLSRQSSDLRRRSDVLSE

SEQ ID NO: 207 - protein, 35 aa, Artificial Sequence
  SYNTHESIZED
TSDHLTEDRSNLSRDRSNLTRTSGHLSRQSSDLRR

SEQ ID NO: 208 - protein, 42 aa, Artificial Sequence
  SYNTHESIZED
RSDNLSVQNATRINRSDALSTDRSTRTKRSDDLSRRNDNRTK

SEQ ID NO: 209 - protein, 35 aa, Artificial Sequence
  SYNTHESIZED
QSGNLARQSGNLARRSDSLSTDNASRIRDRSNLTR

SEQ ID NO: 210 - protein, 35 aa, Artificial Sequence
  SYNTHESIZED
QSSDLSRRNDDRKKRREDLITTSSNLSRQSGHLSR

SEQ ID NO: 211 - DNA, 400 bp, Rattus rattus
tcgaagagctgctggatgaggaccagaaggttcggcccaacgaagaaaaccataaggacgcggacttgtacacttcccgggtgatgctcagcagtcaagtgcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcataga

SEQ ID NO: 212 - DNA, 300 bp, Rattus rattus
gcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcataga

SEQ ID NO: 213 - DNA, 19 bp, Rattus rattus
gccagcacccctgacgcag

SEQ ID NO: 214 - DNA, 17 bp, Rattus rattus
tcgacaagtacctggag

SEQ ID NO: 215 - DNA, 28 bp, Rattus rattus
aagcggttcagggcctgctcccagggtt

SEQ ID NO: 216 - DNA, 28 bp, Rattus rattus
gggattacctgcgctgggtgcagacgct

SEQ ID NO: 217 - DNA, 28 bp, Rattus rattus
cggggtcggagtggcgccgaaccctcat

SEQ ID NO: 218 - DNA, 28 bp, Rattus rattus
cggggtcggagtggcgccgaaccctcat

SEQ ID NO: 219 - protein, 35 aa, Artificial Sequence; identical to SEQ ID NO: 123
  SYNTHESIZED

SEQ ID NO: 220 - protein, 35 aa, Artificial Sequence; identical to SEQ ID NO: 124
  SYNTHESIZED

SEQ ID NO: 221 - protein, 35 aa, Artificial Sequence
  SYNTHESIZED
DRSDLSRDRSHLARRSHNLARRSDDLSKRSAHLSR

SEQ ID NO: 222 - protein, 35 aa, Artificial Sequence
  SYNTHESIZED
RSDNLARQSSDLRRRSSHLSRRSDALSRDRSDLSR

SEQ ID NO: 223 - protein, 35 aa, Artificial Sequence; identical to SEQ ID NO: 123
  SYNTHESIZED

SEQ ID NO: 224 - protein, 35 aa, Artificial Sequence; identical to SEQ ID NO: 124
  SYNTHESIZED

SEQ ID NO: 225 - DNA, 200 bp, Rattus rattus
tacttcaagggctggcagcgatgacccagaagccccacttgagggacagcggaggaccaccgcccaggatagcctgccgggactagccgtcaccaggcgggactggctgatgcgagagaaagagcaattgcaggtgagcggtatagagtcttccagagagggagcggccctctcttatccgtgtgagtcagagcagagaa

SEQ ID NO: 226 - DNA, 28 bp, Rattus rattus
tagtcccggcaggctatcctgggcggtg

SEQ ID NO: 227 - DNA, 28 bp, Rattus rattus
ccgtcaccaggcgggactggctgatgcg

SEQ ID NO: 228 - DNA, 28 bp, Rattus rattus
cggggtcggagtggcgccgaaccctcat

SEQ ID NO: 229 - DNA, 28 bp, Rattus rattus
ccgccgtggggagctgagcgtgtgtgac

SEQ ID NO: 230 - protein, 35 aa, Artificial Sequence
  SYNTHESIZED
NSGNLDKDRSHLSRQSGDLTRRSDTLSQDRSARTR

SEQ ID NO: 231 - protein, 35 aa, Artificial Sequence
  SYNTHESIZED
DRSNLSRRSDNLRERSDHLSADSSTRKTDRSSRKR

SEQ ID NO: 232 - protein, 35 aa, Artificial Sequence; identical to SEQ ID NO: 221
  SYNTHESIZED

SEQ ID NO: 233 - protein, 35 aa, Artificial Sequence; identical to SEQ ID NO: 222
  SYNTHESIZED

SEQ ID NO: 234 - DNA, 28 bp, Rattus rattus
atctggaggaagactggagaacaagagc

SEQ ID NO: 235 - DNA, 28 bp, Rattus rattus
atattctggtaaggagccgggcaagagg

SEQ ID NO: 236 - DNA, 28 bp, Rattus rattus
atgacgtcaaggaagaagtctgcagggt

SEQ ID NO: 237 - DNA, 28 bp, Rattus rattus
acggagattcttcggctgtaatgttaaa

SEQ ID NO: 238 - DNA, 20 bp, Rattus rattus
ccccagtttgtggtctgcca

SEQ ID NO: 239 - DNA, 18 bp, Rattus rattus
gctaaaggtgaagatcta

SEQ ID NO: 240 - protein, 42 aa, Artificial Sequence
  SYNTHESIZED
QSGNLARQSGHLSRDRSALSRQSGNLARRSDALSRRSDALTQ

SEQ ID NO: 241 - protein, 42 aa, Artificial Sequence
  SYNTHESIZED
RSDHLSETSSDRTKRSDHLSAQSGSLTRRSDVLSEHSNARKT

SEQ ID NO: 242 - protein, 35 aa, Artificial Sequence; identical to SEQ ID NO: 209
  SYNTHESIZED

SEQ ID NO: 243 - protein, 35 aa, Artificial Sequence; identical to SEQ ID NO: 210
  SYNTHESIZED

(19) United States
(12) Patent Application Publication
(10) Pub. No.: US 2012/0192298 A1
(43) Pub. Date: Jul. 26, 2012

(54) METHOD FOR GENOME EDITING

(75) Inventors: Edward Weinstein, St. Louis, MO (US); Xiaoxia Cui, St. Louis, MO (US); Phil Simmons, St. Louis, MO (US)

(73) Assignee: SIGMA ALDRICH CO. LLC, St. Louis, MO (US)

(21) Appl. No.: 13/386,394

(22) PCT Filed: Jul. 23, 2010
(86) PCT No.: PCT/US10/43167

§ 371 Date: Apr. 13, 2012

Related U.S. Application Data
(63) Continuation-in-part of application No. 12/842,217, filed on Jul. 23, 2010.
Application No. 12/842,217 is a continuation-in-part of application No. 12/842,219, filed on Jul. 23, 2010.
Application No. 12/842,219 is a continuation-in-part of application No. 12/842,269, filed on Jul. 23, 2010.
Application No. 12/842,269 is a continuation-in-part of application No. 12/842,208, filed on Jul. 23, 2010.
Application No. 12/842,208 is a continuation-in-part of application No. 12/842,204, filed on Jul. 23, 2010.
Application No. 12/842,204 is a continuation-in-part of application No. 12/842,198, filed on Jul. 23, 2010.
Application No. 12/842,198 is a continuation-in-part of application No. 12/842,188, filed on Jul. 23, 2010.
Application No. 12/842,188 is a continuation-in-part of application No. 12/842,719, filed on Jul. 23, 2010.
Application No. 12/842,719 is a continuation-in-part of application No. 12/842,713, filed on Jul. 23, 2010.
Application No. 12/842,713 is a continuation-in-part of application No. 12/842,708, filed on Jul. 23, 2010.
Application No. 12/842,708 is a continuation-in-part of application No. 12/842,694, filed on Jul. 23, 2010.
Application No. 12/842,694 is a continuation-in-part of application No. 12/842,678, filed on Jul. 23, 2010.
Application No. 12/842,678 is a continuation-in-part of application No. 12/842,666, filed on Jul. 23, 2010.
Application No. 12/842,666 is a continuation-in-part of application No. 12/842,620, filed on Jul. 23, 2010.
Application No. 12/842,620 is a continuation-in-part of application No. 12/842,578, filed on Jul. 23, 2010.
Application No. 12/842,578 is a continuation-in-part of application No. 12/842,897, filed on Jul. 23, 2010.
Application No. 12/842,897 is a continuation-in-part of application No. 12/842,893, filed on Jul. 23, 2010.
Application No. 12/842,893 is a continuation-in-part of application No. 12/842,886, filed on Jul. 23, 2010.
Application No. 12/842,886 is a continuation-in-part of application No. 12/842,839, filed on Jul. 23, 2010.
Application No. 12/842,839 is a continuation-in-part of application No. 12/842,980, filed on Jul. 23, 2010.
Application No. 12/842,980 is a continuation-in-part of application No. 12/842,978, filed on Jul. 23, 2010.
Application No. 12/842,978 is a continuation-in-part of application No. 12/842,976, filed on Jul. 23, 2010.
Application No. 12/842,976 is a continuation-in-part of application No. 12/842,982, filed on Jul. 23, 2010.
Application No. 12/842,982 is a continuation-in-part of application No. 12/842,994, filed on Jul. 23, 2010.
Application No. 12/842,994 is a continuation-in-part of application No. 12/842,993, filed on Jul. 23, 2010.
Application No. 12/842,993 is a continuation-in-part of application No. 12/842,991, filed on Jul. 23, 2010.
Application No. 12/842,991 is a continuation-in-part of application No. 12/842,999, filed on Jul. 23, 2010.
Application No. 12/842,999 is a continuation-in-part of application No. 12/843,000, filed on Jul. 23, 2010.
(60) Provisional application No. 61/228,419, filed on Jul. 24, 2009.
(60) Provisional application No. 61/232,620, filed on Aug. 10, 2009.
(60) Provisional application No. 61/245,877, filed on Sep. 25, 2009.
(60) Provisional application No. 61/263,696, filed on Nov. 23, 2009.
(60) Provisional application No. 61/263,904, filed on Nov. 24, 2009.
(60) Provisional application No. 61/336,000, filed on Jan. 14, 2010.
(60) Provisional application No. 61/308,089, filed on Feb. 25, 2010.
(60) Provisional application No. 61/309,729, filed on Mar. 2, 2010.
(60) Provisional application No. 61/323,702, filed on Apr. 13, 2010.
(60) Provisional application No. 61/323,698, filed on Apr. 13, 2010.
(60) Provisional application No. 61/323,719, filed on Apr. 13, 2010.
(60) Provisional application No. 61/343,287, filed on Apr. 26, 2010.

(51) Int. Cl. C12N 15/85; C12N 5/10; A01K 67/027

(52) U.S. Cl. 800/14; 435/455; 435/325

(57) ABSTRACT

The present invention encompasses a method for creating an animal or cell with at least one chromosomal edit. In particular, the invention relates to the use of targeted zinc finger nucleases to edit chromosomal sequences. The invention further encompasses an animal or a cell created by a method of the invention.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### FIELD OF THE INVENTION

The invention encompasses a method for creating an animal or cell with at least one chromosomal edit. In particular, the invention relates to the use of targeted zinc finger nucleases to edit chromosomal sequences.

### BACKGROUND OF THE INVENTION

Rational genome engineering has enormous potential across basic research, drug discovery, and cell-based medicines. Existing methods for targeted gene knock-out or site-specific gene insertion rely on homologous recombination. The low rate of spontaneous recombination in certain cell types, however, has been an enormous hurdle to universal genome editing. The scale of screening effort and the time required to isolate the targeted event was prohibitive. Thus, there exists a strong need for a technology that can rapidly achieve genomic editing in most cell types with high speed and efficiency, so as to greatly reduce the overall engineering effort.

### SUMMARY OF THE INVENTION

One aspect of the present invention encompasses a method for editing a chromosomal sequence. The method comprises, in part, (a) introducing into a cell comprising the chromosomal sequence at least one nucleic acid encoding a zinc finger nuclease that recognizes a target sequence in the chromosomal sequence and is able to cleave a cleavage site in the chromosomal sequence, and, optionally, (i) at least one donor polynucleotide comprising a donor sequence for integration, an upstream sequence, and a downstream sequence, wherein the donor sequence is flanked by the upstream sequence and the downstream sequence, and wherein the upstream sequence and the downstream sequence share substantial sequence identity with either side of the cleavage site, or (ii) at least one exchange polynucleotide comprising an exchange sequence that is substantially identical to a portion of the chromosomal sequence at the cleavage site, and further comprising at least one nucleotide change; and (b) culturing the cell to allow expression of the zinc finger nuclease such that the zinc finger nuclease introduces a double-stranded break into the chromosomal sequence at the cleavage site, and wherein the double-stranded break is repaired by (i) a non-homologous end-joining repair process such that a mutation is introduced into the chromosomal sequence, or optionally (ii) a homology-directed repair process such that the donor sequence is integrated into the chromosomal sequence or the exchange sequence is exchanged with the portion of the chromosomal sequence.

Another aspect of the present invention encompasses a non-human animal. The non-human animal may be created in part, by (a) introducing into a cell comprising the chromosomal sequence at least one nucleic acid encoding a zinc finger nuclease that recognizes a target sequence in the chromosomal sequence and is able to cleave a cleavage site in the chromosomal sequence, and, optionally, (i) at least one donor polynucleotide comprising a donor sequence for integration, an upstream sequence, and a downstream sequence, wherein the donor sequence is flanked by the upstream sequence and the downstream sequence, and wherein the upstream sequence and the downstream sequence share substantial sequence identity with either side of the cleavage site, or (ii) at least one exchange polynucleotide comprising an exchange sequence that is substantially identical to a portion of the chromosomal sequence at the cleavage site, and further comprising at least one nucleotide change; and (b) culturing the cell to allow expression of the zinc finger nuclease such that the zinc finger nuclease introduces a double-stranded break into the chromosomal sequence at the cleavage site, and wherein the double-stranded break is repaired by (i) a non-homologous end-joining repair process such that a mutation is introduced into the chromosomal sequence, or optionally (ii) a homology-directed repair process such that the donor sequence is integrated into the chromosomal sequence or the exchange sequence is exchanged with the portion of the chromosomal sequence.

Yet another aspect of the present invention encompasses a cell. The cell may be created in part, by in part, by (a) introducing into the cell comprising the chromosomal sequence at least one nucleic acid encoding a zinc finger nuclease that recognizes a target sequence in the chromosomal sequence and is able to cleave a cleavage site in the chromosomal sequence, and, optionally, (i) at least one donor polynucleotide comprising a donor sequence for integration, an upstream sequence, and a downstream sequence, wherein the donor sequence is flanked by the upstream sequence and the downstream sequence, and wherein the upstream sequence and the downstream sequence share substantial sequence identity with either side of the cleavage site, or (ii) at least one exchange polynucleotide comprising an exchange sequence that is substantially identical to a portion of the chromosomal sequence at the cleavage site, and further comprising at least one nucleotide change; and (b) culturing the cell to allow expression of the zinc finger nuclease such that the zinc finger nuclease introduces a double-stranded break into the chromosomal sequence at the cleavage site, and wherein the double-stranded break is repaired by (i) a non-homologous end-joining repair process such that a mutation is introduced into the chromosomal sequence, or optionally (ii) a homology-directed repair process such that the donor sequence is integrated into the chromosomal sequence or the exchange sequence is exchanged with the portion of the chromosomal sequence.

A further aspect of the present invention encompasses an embryo. Typically, the embryo comprises at least one nucleic acid encoding a zinc finger nuclease that recognizes a target sequence in the chromosomal sequence and is able to cleave a cleavage site in the chromosomal sequence, and, optionally, (i) at least one donor polynucleotide comprising a donor sequence for integration, an upstream sequence and a downstream sequence, wherein the donor sequence is flanked by the upstream sequence and the downstream sequence and wherein the upstream sequence and the downstream sequence share substantial sequence identity with either side of the cleavage site, or (ii) at least one exchange polynucleotide comprising an exchange sequence that is substantially identical to a portion of the chromosomal sequence at the cleavage site and which further comprises at least one nucleotide change.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

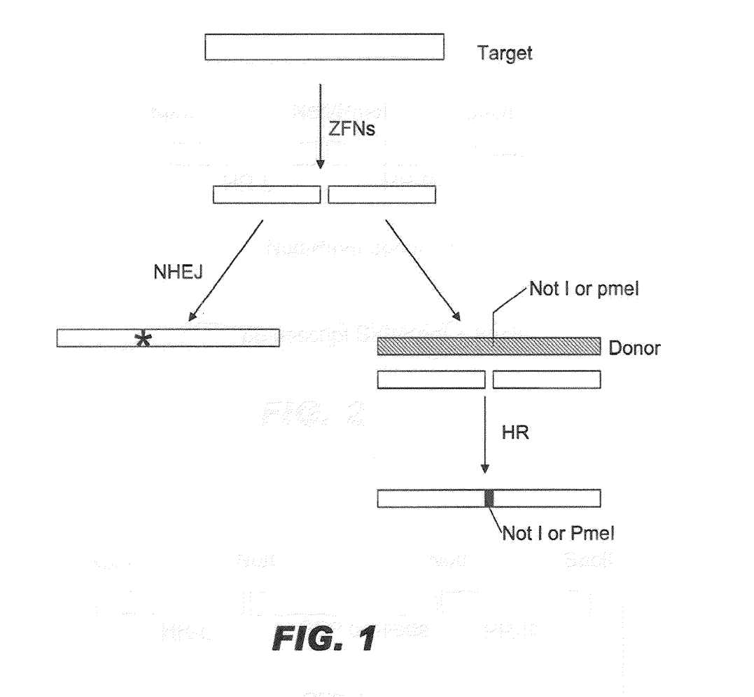

FIG. 1 is a schematic depicting the repair outcomes after a targeted ZFN-induced double stranded break. Shaded bars represent the donor fragment, whereas white bars depict target site for ZFN double stranded break.

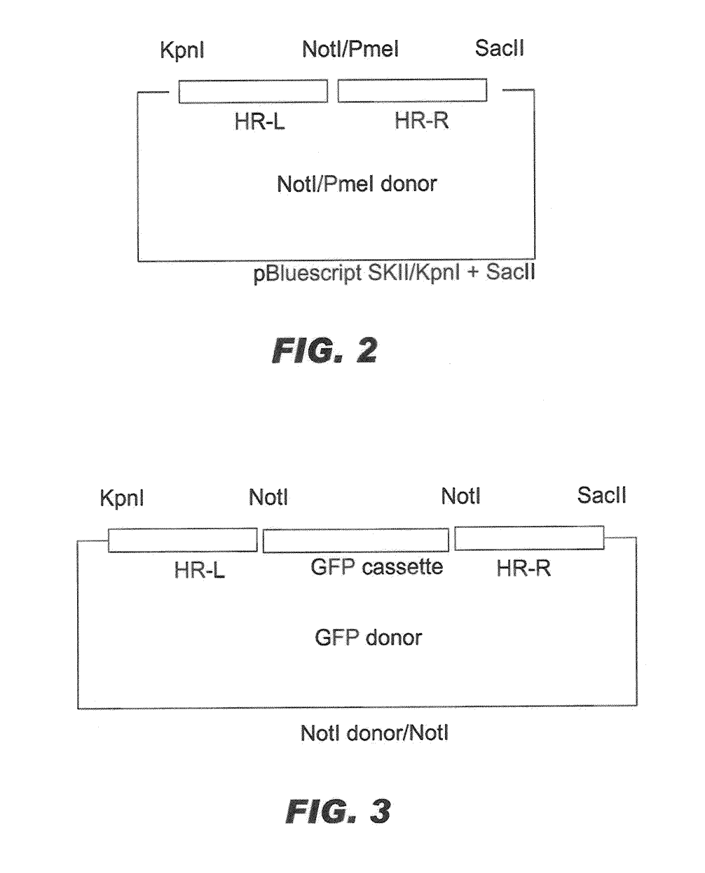

FIG. 2 is a schematic depicting the construction of RFLP donor plasmids. Shown, are the plasmid, and left and right PCR-amplified fragments homologous to the integration target site. Restriction enzymes used for cloning are denoted. The left fragment used KpnI and NotI or PmeI. The right fragment used NotI or PmeI and SacII.

FIG. 3 is a schematic depicting the construction of GFP-expressing donor plasmids. The GFP cassette was PCR amplified from an existing plasmid and cloned into the NotI RFLP donor using a NotI site.

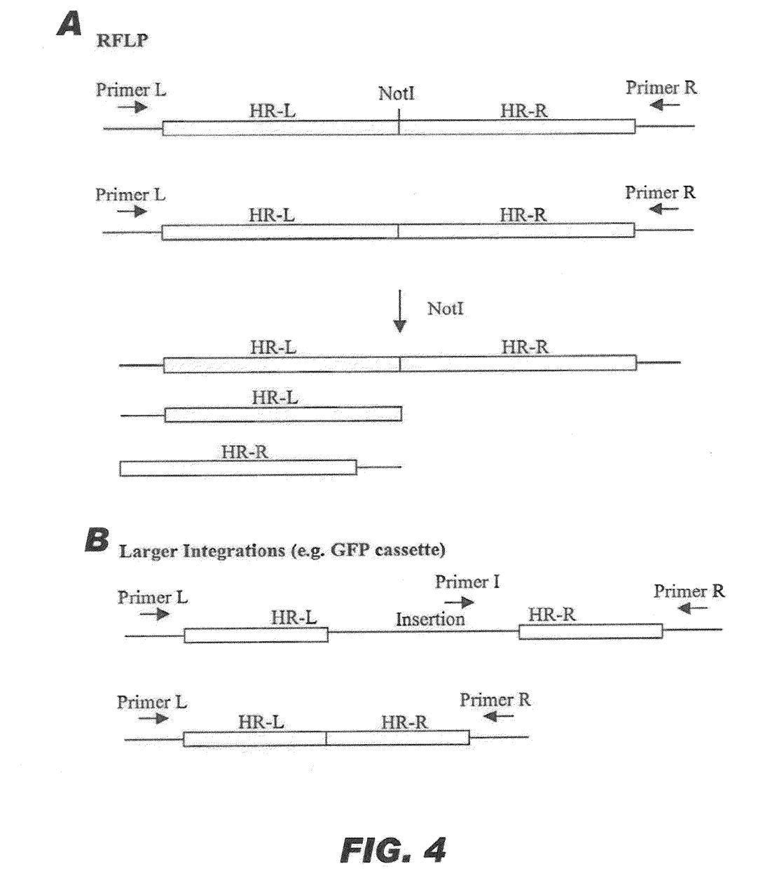

FIG. 4 is a schematic depicting methods of detecting (A) RFLP integration and restriction enzyme digestion and (B) integration of the GFP expression cassette using PCR amplification.

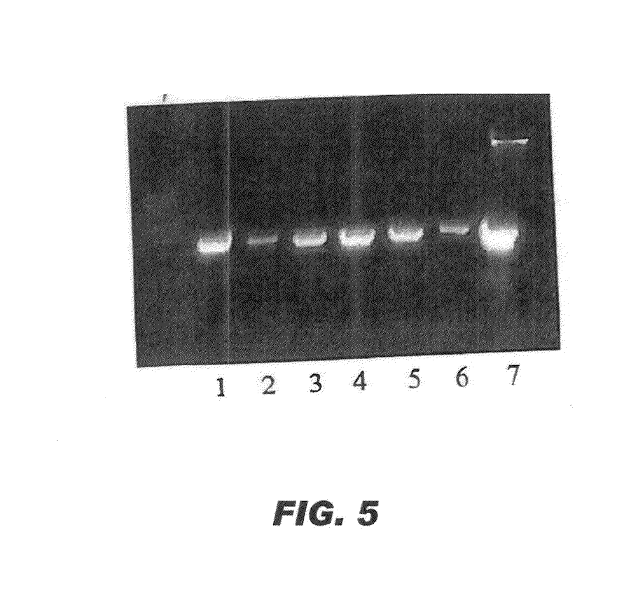

FIG. 5 is a photographic image of fluorescently stained PCR fragments resolved on an agarose gel. The leftmost lane contains a DNA ladder. Lanes 1 to 6 contain PCR fragments amplified using mouse Mdr1a-specific primers from a whole or a fraction of a mouse blastocyst. Lanes 1 and 2 were amplified from ⅚ and ⅙ of a blastocyst, respectively. Lane 3 was from one whole blastocyst. Lanes 4 to 6 were from ½, ⅓, and ⅙ of the same blastocyst, respective. Lane 7 contains a positive control PCR fragment amplified using the same primers from extracted mouse toe DNA.

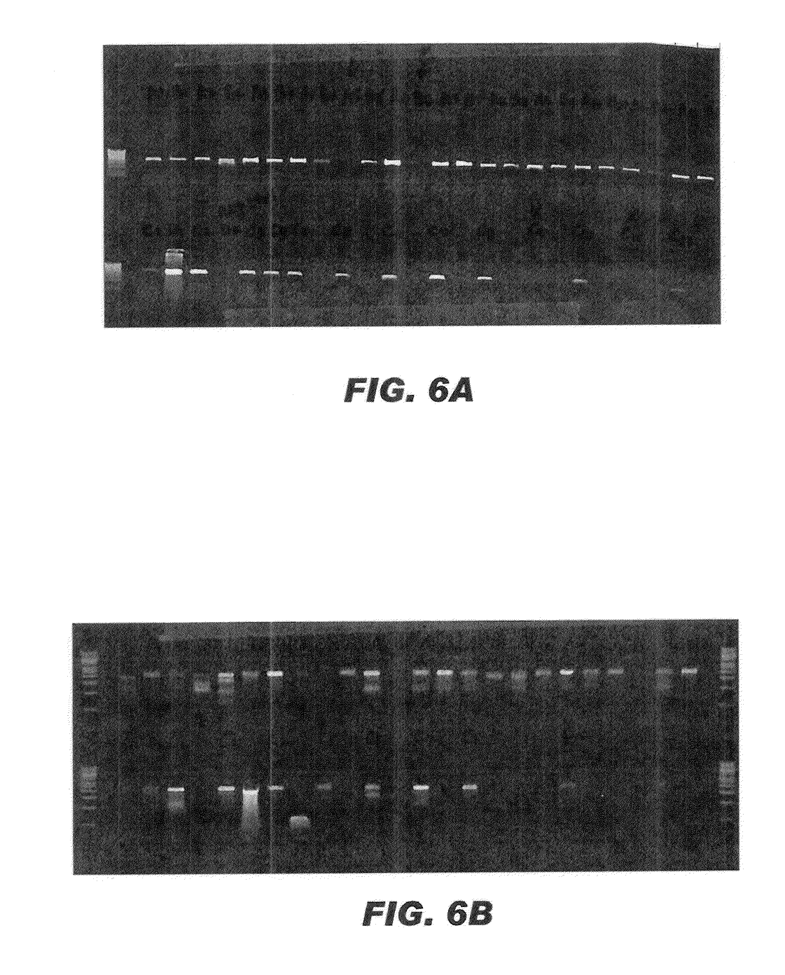

FIG. 6 is a photographic image of fluorescently stained DNA fragments resolved on an agarose gel. The leftmost lanes contain a DNA ladder. (A) Lanes 1 to 39 contain PCR fragments amplified using mMdr1a-specific primers from 37 mouse embryos cultured in vitro after being microinjected with ZFN RNA against mouse Mdr1a and RFLP donor with NotI site, along with one positive and negative control for PCR amplification. (B) Lanes 1 to 39 contain the PCR fragments in (A) after performing the Surveyor's mutation detection assay.

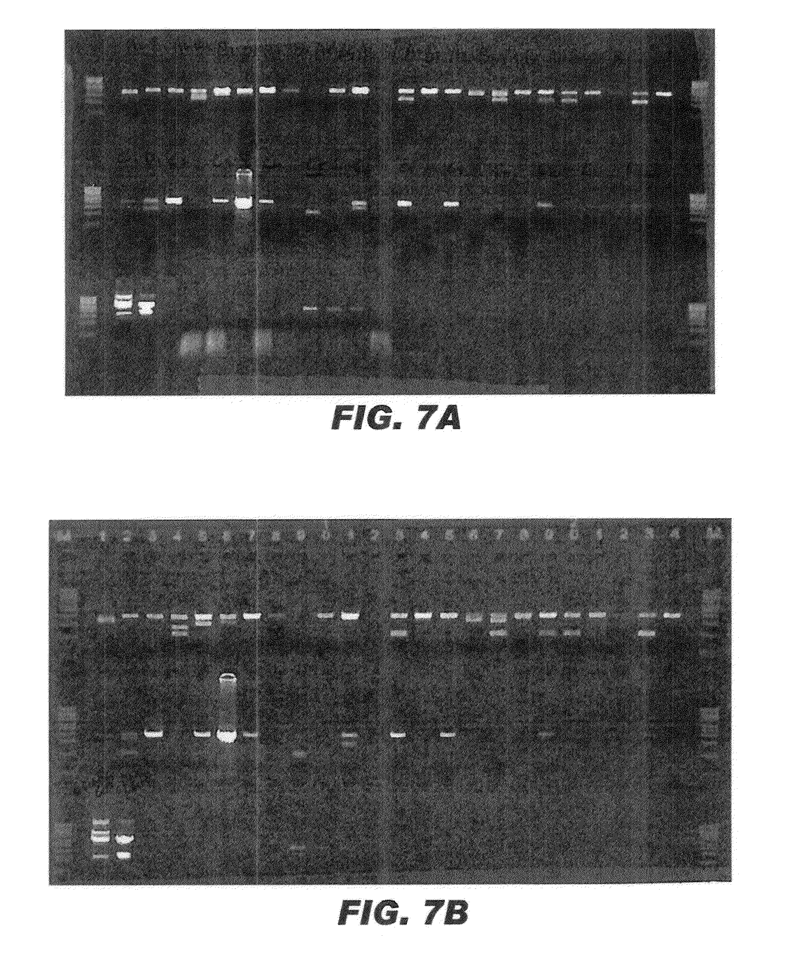

FIG. 7 is a photographic image of fluorescently stained DNA fragments resolved on an agarose gel. The leftmost and rightmost lanes contain a DNA ladder. (A) Lanes contain PCR fragments amplified using mMdr1a-specific primers from mouse embryos in FIG. 6, and digested with NotI without purifying the PCR product. FIG. 7B is a longer run of the same gel in FIG. 7A. The uncut PCR products are around 1.8 kb, and the digested products are two bands around 900 bp.

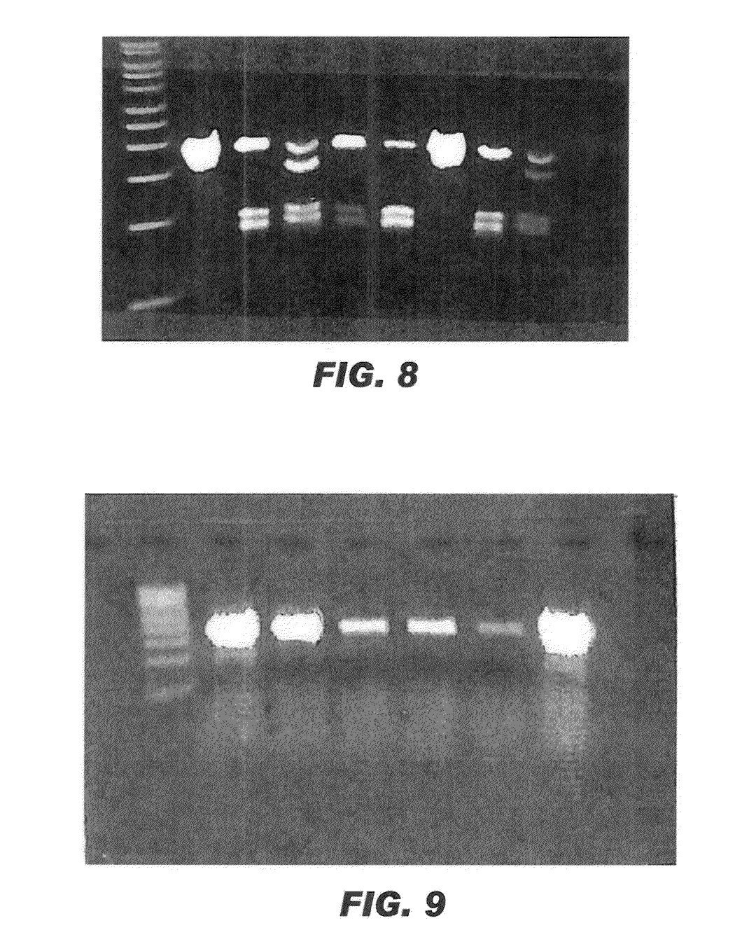

FIG. 8 is a photographic image of fluorescently stained DNA fragments resolved on an agarose gel. The leftmost lane contains a DNA ladder. Lanes 1 to 6 contain some of the PCR fragments from FIG. 7 digested with NotI after the PCR products were column purified so that NotI can work in its optimal buffer. Lines 7 and 8 are two of the samples digested with NotI as in FIG. 7. This gel shows NotI digestion in PCR reactions was complete.

FIG. 9 is a photographic image of fluorescently stained PCR fragments resolved on an agarose gel. The leftmost lane contains a DNA ladder. Lanes 1 to 5 contain PCR fragments amplified using PXR-specific primers from 1, ½, ⅙, 1/10, 1/30 of a rat blastocyst. Lane 6 is a positive control amplified using the same primers from purified Sprague Dawley genomic DNA.

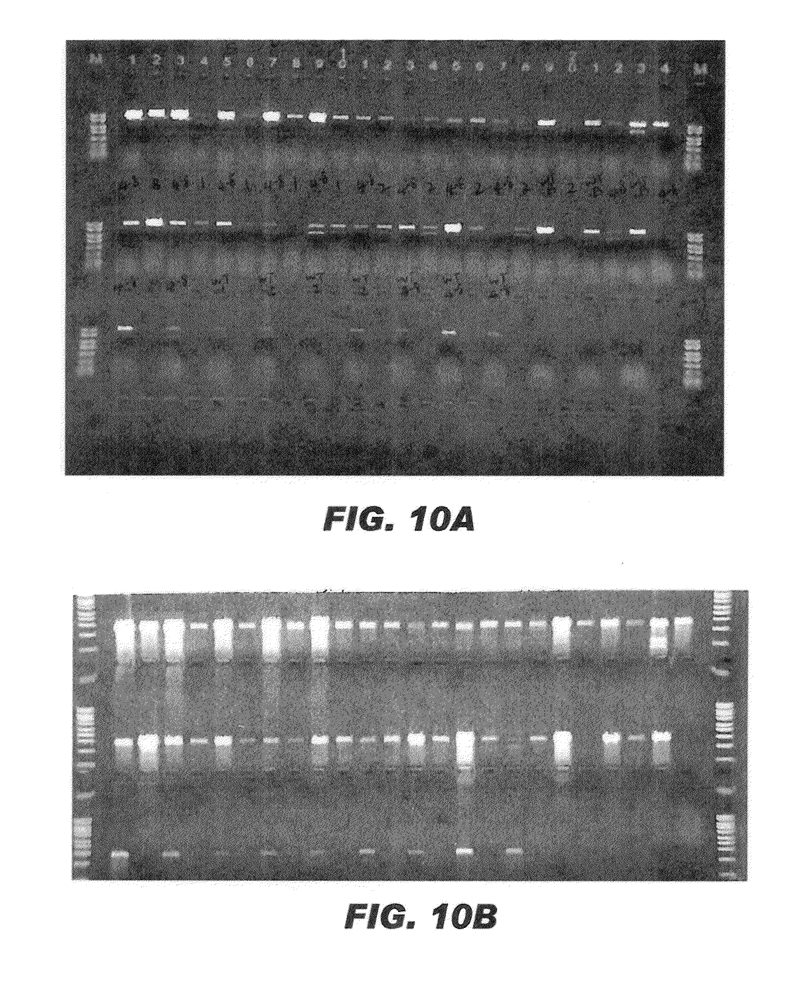

FIG. 10 is a photographic image of fluorescently stained DNA fragments resolved on an agarose gel. The leftmost and rightmost lanes contain a DNA ladder. (A) Lanes contain PCR fragments amplified from rat embryos cultured in vitro after microinjection of PXR ZFN mRNA and the NotI RFLP donor, using PXR-specific primers and digested with NotI. (B) Lanes contain the same PCR fragments as in FIG. 10A after performing the Surveyor's mutation detection assay.

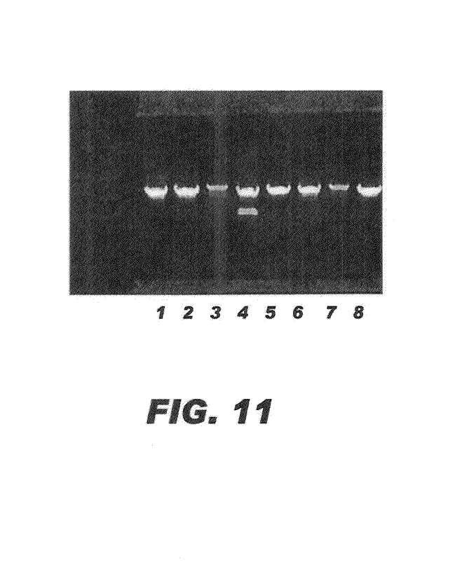

FIG. 11 is a photographic image of fluorescently stained DNA fragments resolved on an agarose gel. The first 4 lanes are PCR amplified from 4 well developed fetus at 12.5 days post conception from embryos injected with mMdr1a ZFN mRNA with the NotI RFLP donor. The PCR was digested with NotI. Lane 4 is positive one. Lanes 5-8 are 4 decidua, aborted implantations. All four were negative.

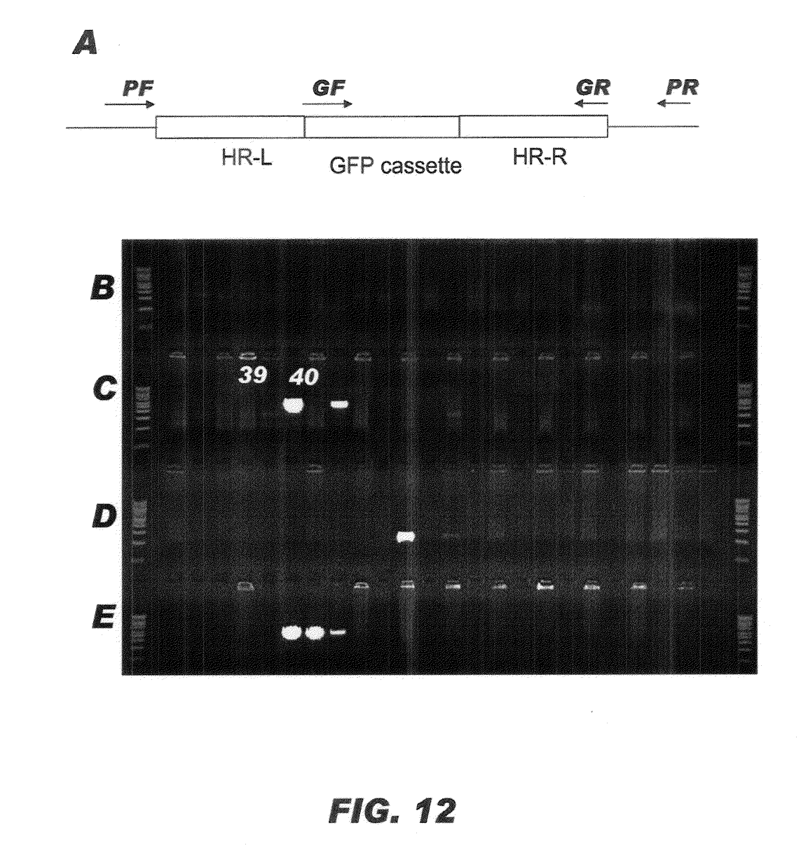

FIG. 12 is a schematic and photographic image of fluorescently stained DNA fragments resolved on an agarose gel. (A) A schematic showing the location of the primers used. Panels (B) and (C) show results from primers PF and GR. Panels (D) and (E) show results from primers PR+GF. Expected fragment size is 2.4 kb. Two out of forty fetuses were positive for GFP.

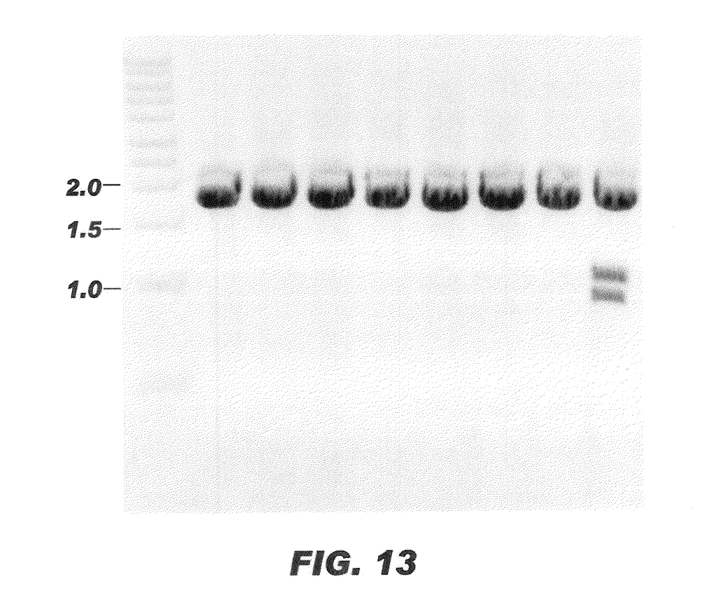

FIG. 13 is a photographic image of DNA fragments resolved on an agarose gel. Lane 8 represents a 13 dpc fetus positive for the NotI site.

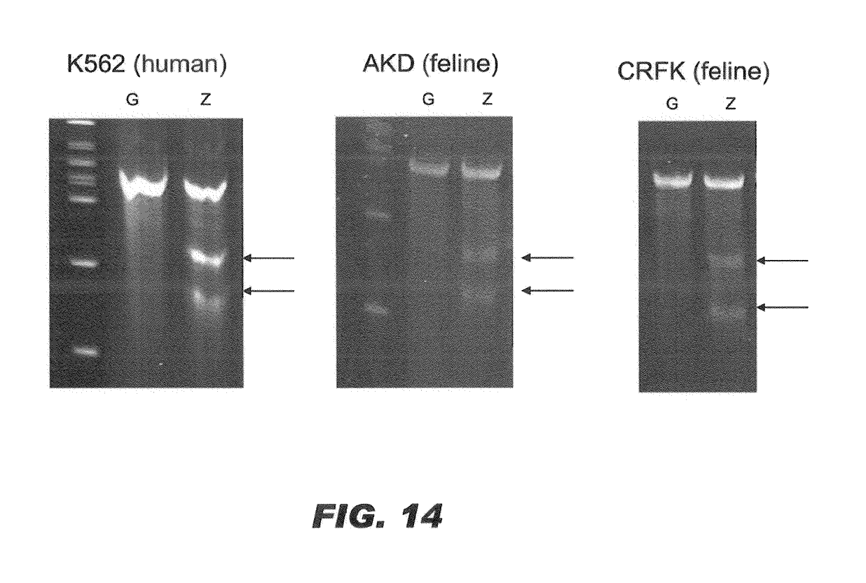

FIG. 14 illustrates ZFN-mediated cleavage of SMAD4 in human and feline cells, as detected by a Cel-1 surveyor nuclease assay. G=GFP (no ZFN control). Z=SMAD4 ZFN (191160/19159). Arrows denote cleavage products.

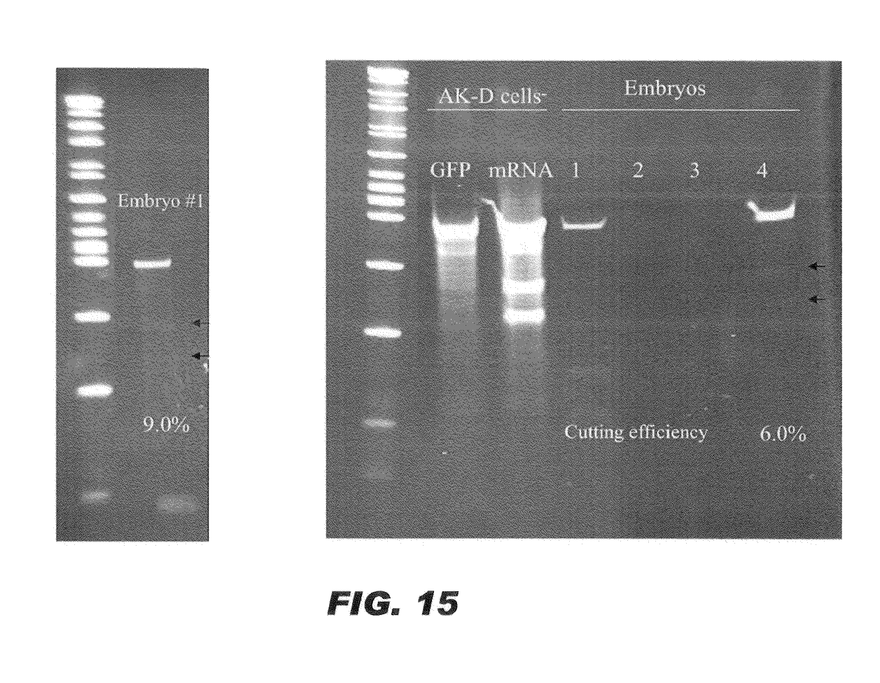

FIG. 15 depicts Cel-1 assays confirming SMAD4 ZFN activity in cat embryos.

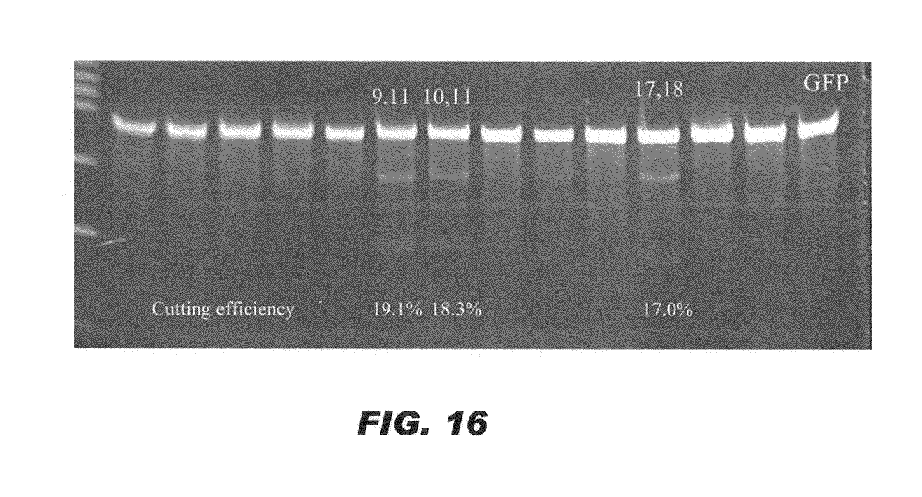

FIG. 16 illustrates cleavage of Fel d1 in AKD cells. Presented is Cel-1 screening of the Fel d1 ZFN pair 17, 18 cleavage of chain 1-exon 1.

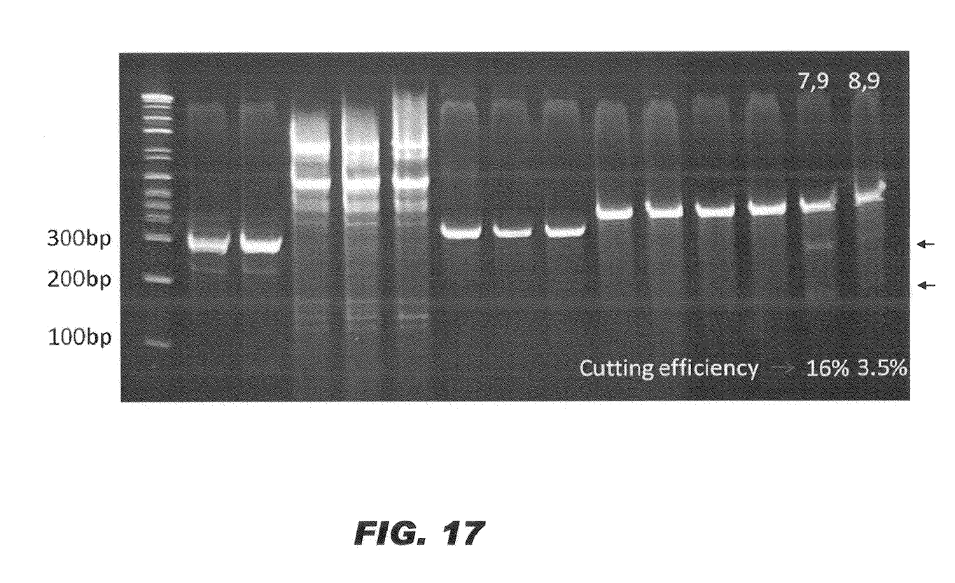

FIG. 17 illustrates cleavage of Fel d1 chain 1-exon 2 in AKD cells by the Fel d1 ZFN pair 7, 9.

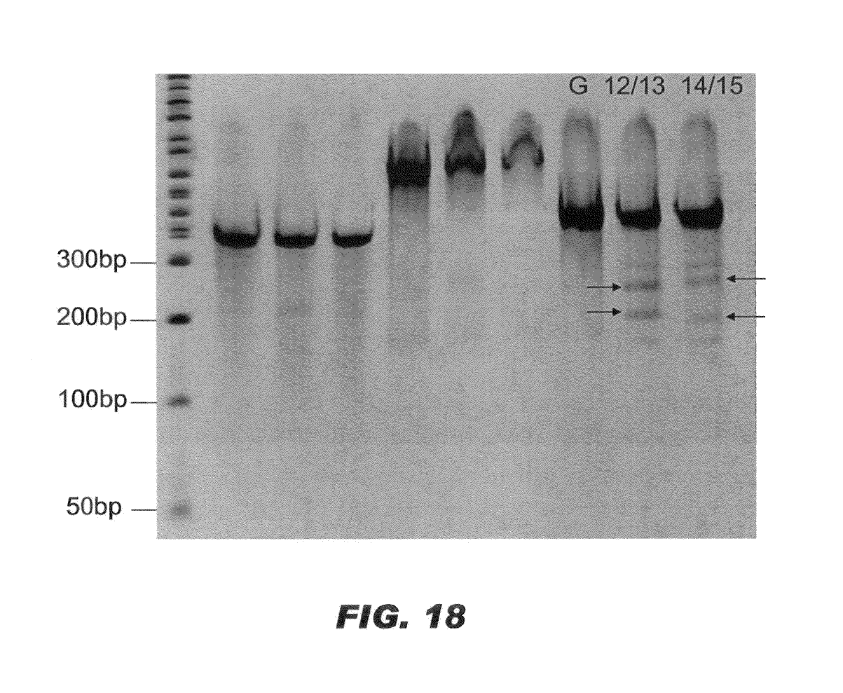

FIG. 18 depicts Cel-1 analysis of the Fel d1 ZFN pair 12/13 cleavage of chain 1-exon 2 in AKD cells.

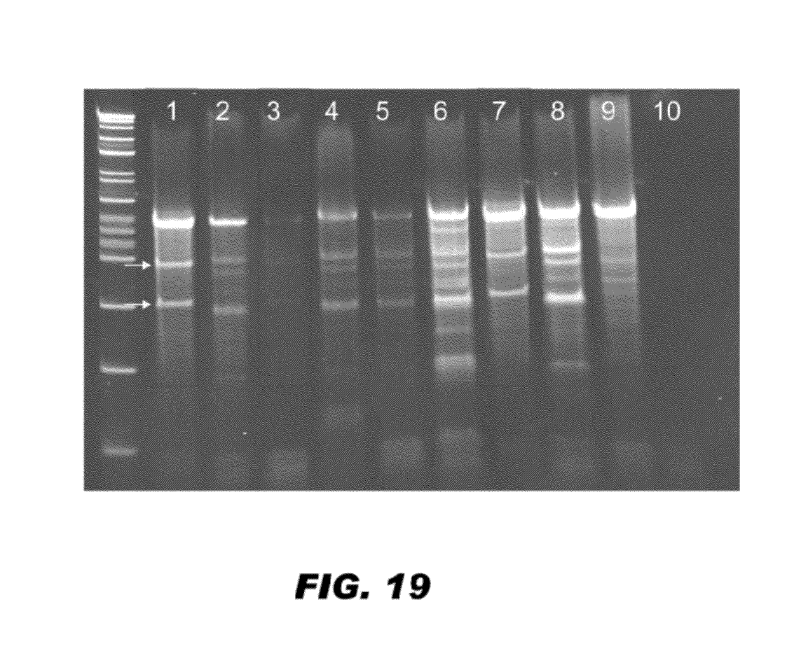

FIG. 19 illustrates cleavage of Fel d1 locus in cat embryos by ZFN pairs 17, 18 and 12, 13. Lanes 1, 2, 7, and 8 contain samples from individual blastocysts derived from embryos injected with 40 ng/μL of ZFNs. Lane 3 presents a sample from a blastocyst derived an embryo injected with 20 ng/μL of ZFNs. Lanes 4, 9, and 10 contain samples from individual morulas derived from embryos injected with 40 ng/μL of ZFNs. Lane 3 presents a sample from a morula derived an embryo injected with 20 ng/μL of ZFNs. Lane 6 presents a sample from a control blastocyst.

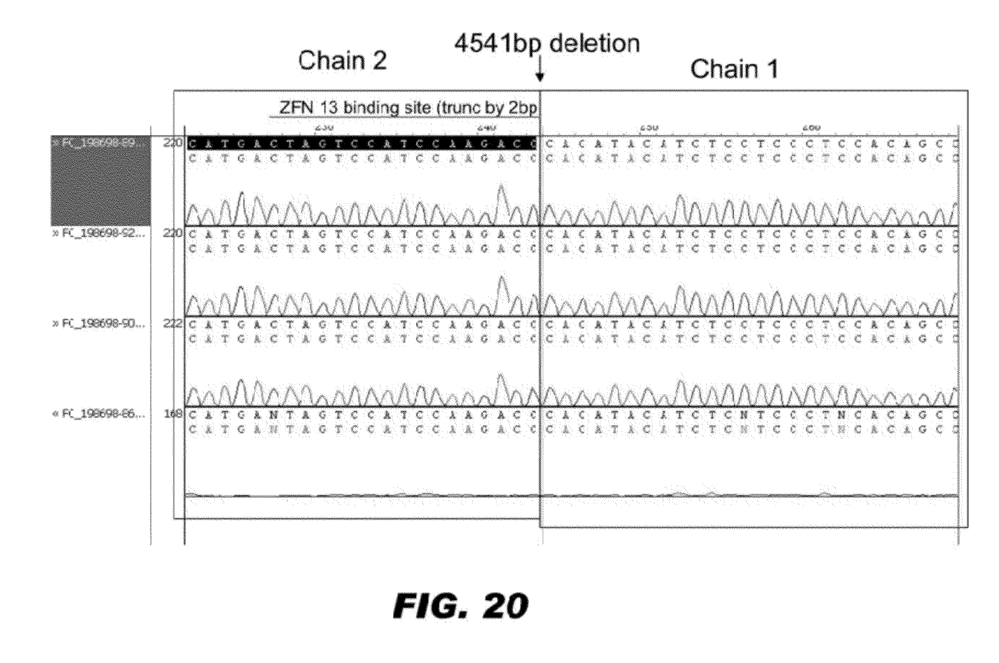

FIG. 20 presents the DNA sequence of an edited Fel dl locus comprising a 4541 bp deletion (SEQ ID NO:51) between the regions coding for chain 2 and chain 1.

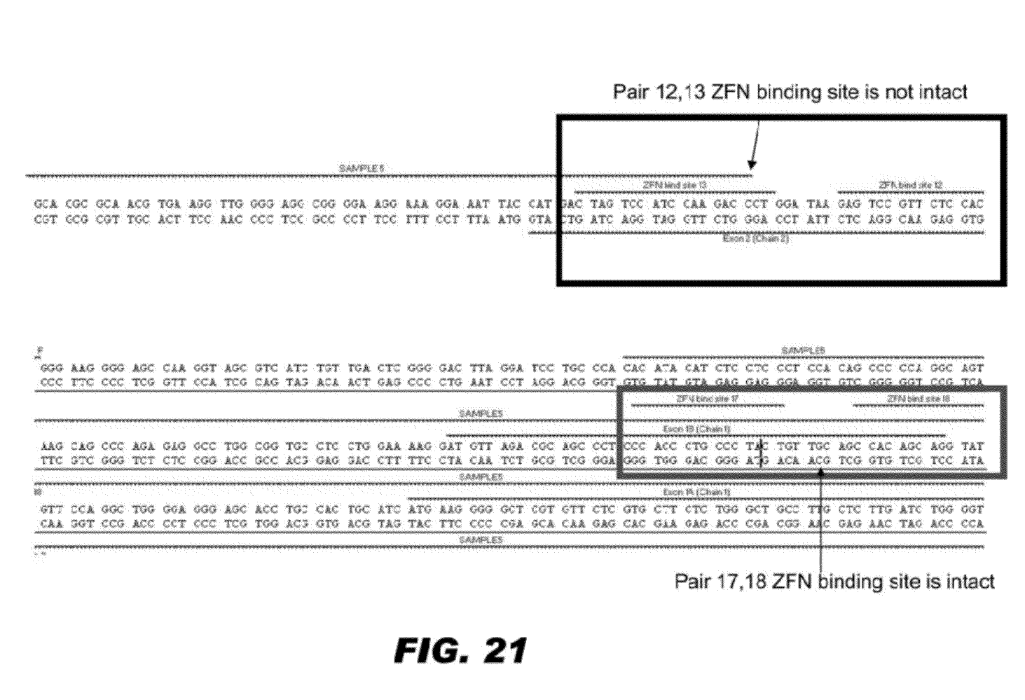

FIG. 21 aligns the edited Fel d1 locus (designated by red dotted line, labeled “sample 5”) comprising the 4541 bp deletion with the sequence of the wild-type Fel d1 locus (SEQ ID NO:52). In the edited sample, the binding site for ZFN 13 is truncated (and the binding sire for ZFN 12 is missing), but the binding site for ZFN pair 17, 18 is intact.

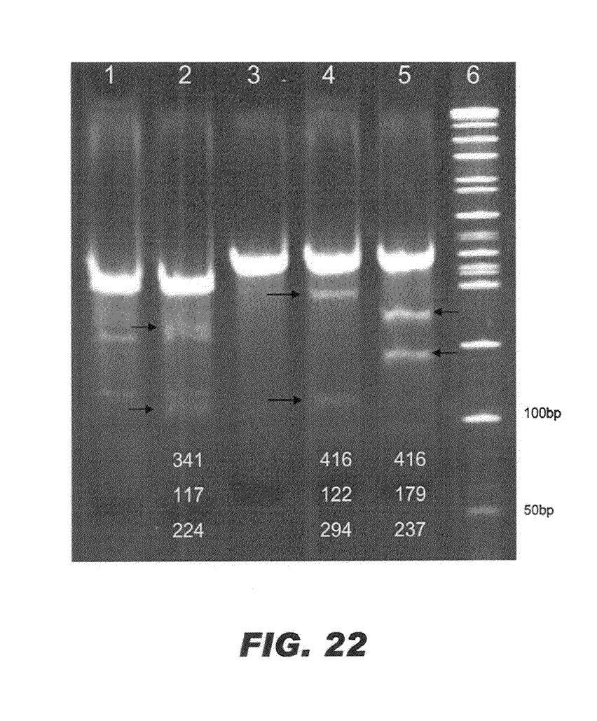

FIG. 22 depicts cleavage of the cauxin locus by cauxin ZFN pair 1/2 (lane 2), ZFN pair 9/10 (lane 4), and ZFN pair 17/18 (lane 5) in AKD cells. Lanes 1 and 3 contain samples from control (GFP) cells.

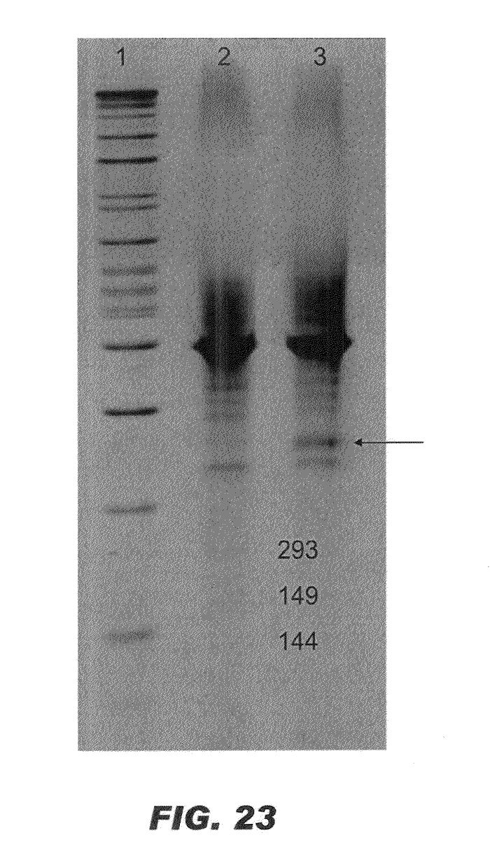

FIG. 23 illustrates cleavage of the cauxin locus by cauxin ZFN pair 29/30 (lane 2). Lane 2 contains a control (GFP) sample.

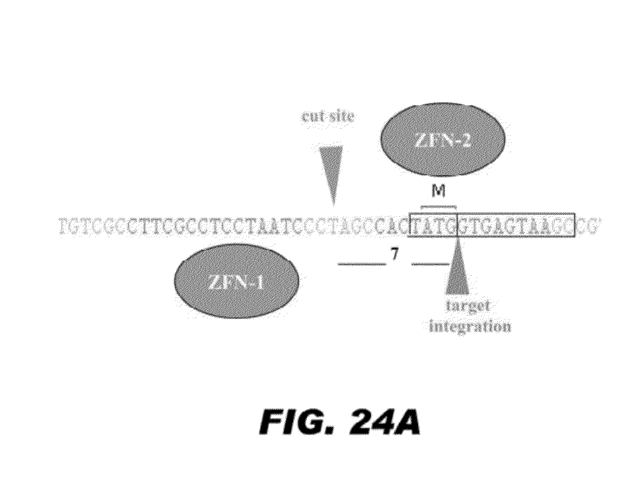

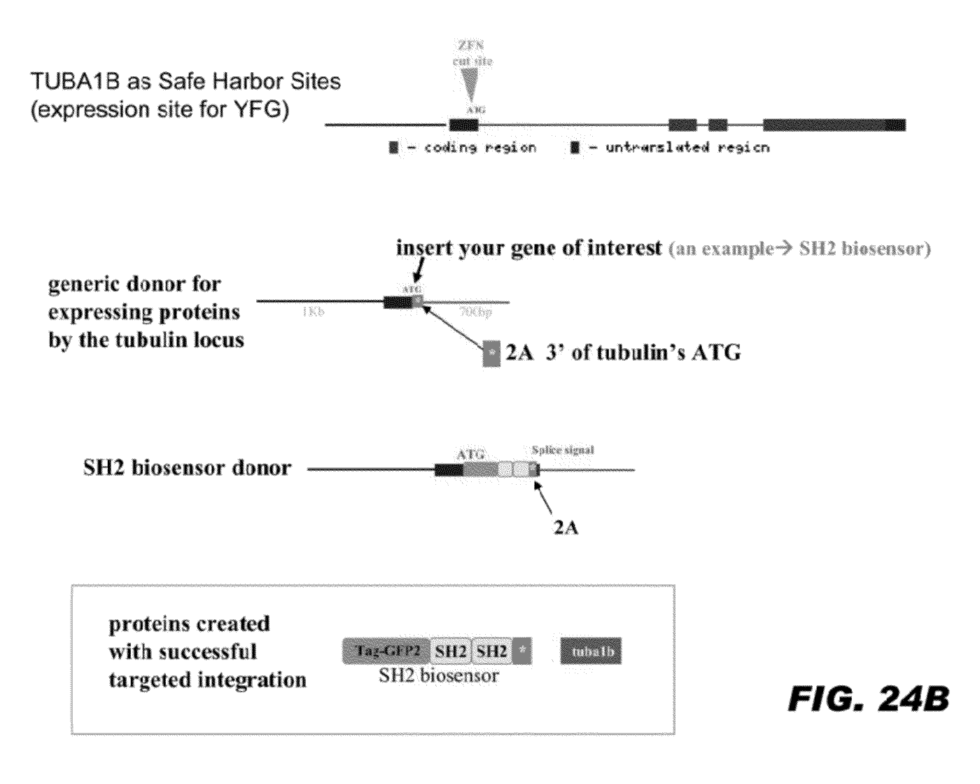

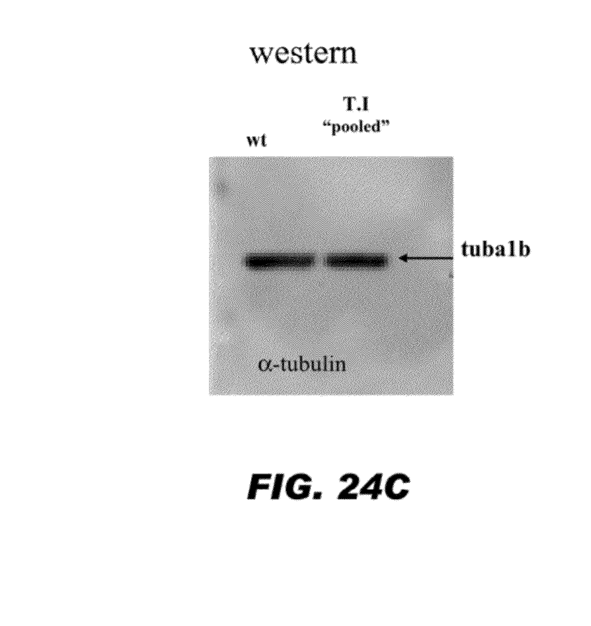

FIG. 24 depicts integration at the TUBA1B locus. (A) is a schematic showing the chromosome sequence (SEQ ID NO:85) at the target region for integration of the heterologous coding sequence, ZFN binding sites (yellow sequence) on the chromosome target region, the ZFN cut site (yellow arrow), and the integration site (green arrow). (B) presents schematics of the TUBA1B locus, site of integration, design of the SH2 biosensor, and the proteins expressed after successful integration. (C) presents an image of a Western blot of wild-type and integrated cells.

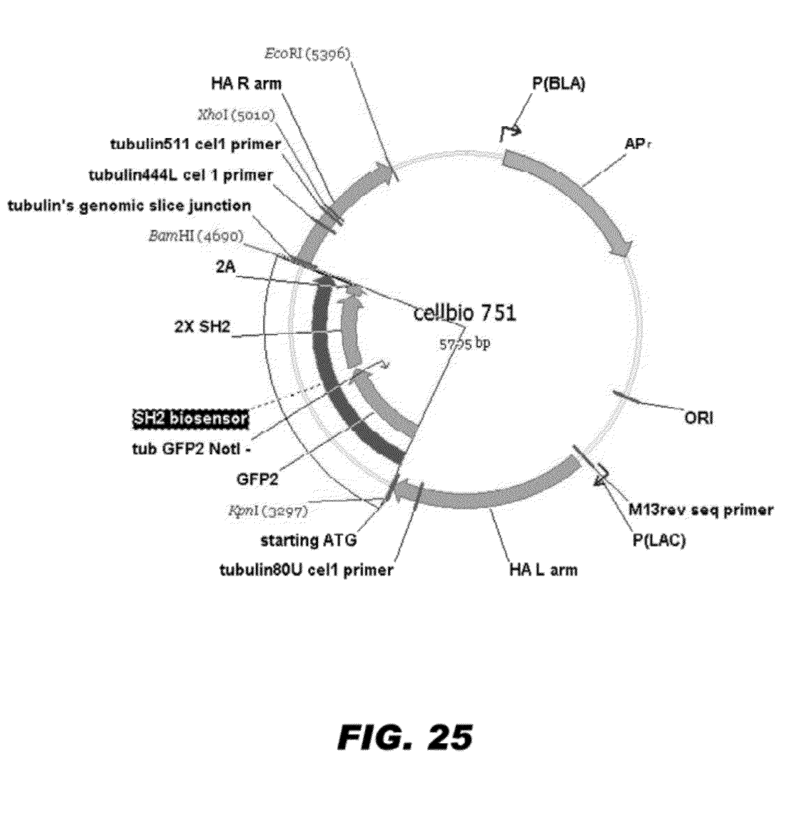

FIG. 25 depicts the map of a donor plasmid comprising the SH2 biosensor sequence flanked by TUBA1A sequences at the target region.

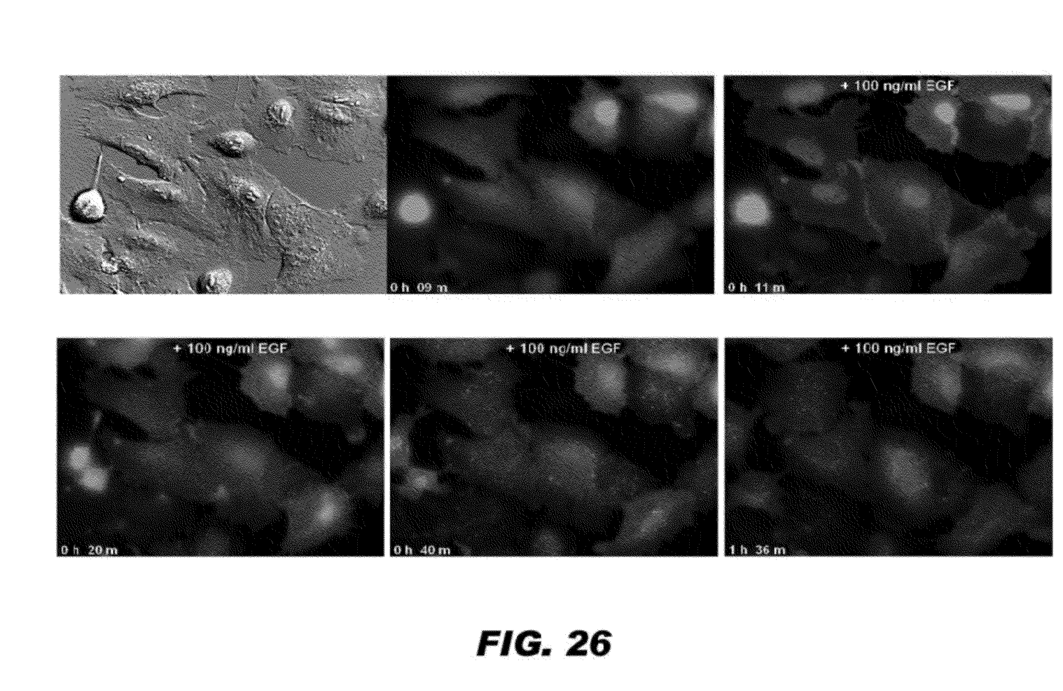

FIG. 26 presents differential interference contrast (DIC) and fluorescence microscopy images of individual isolated cell clones expressing the GFP-2xSH2-Grb1-2A protein. Fluorescent images show a time course of biosensor translocation after exposure to 100 ng/mL of EGF.

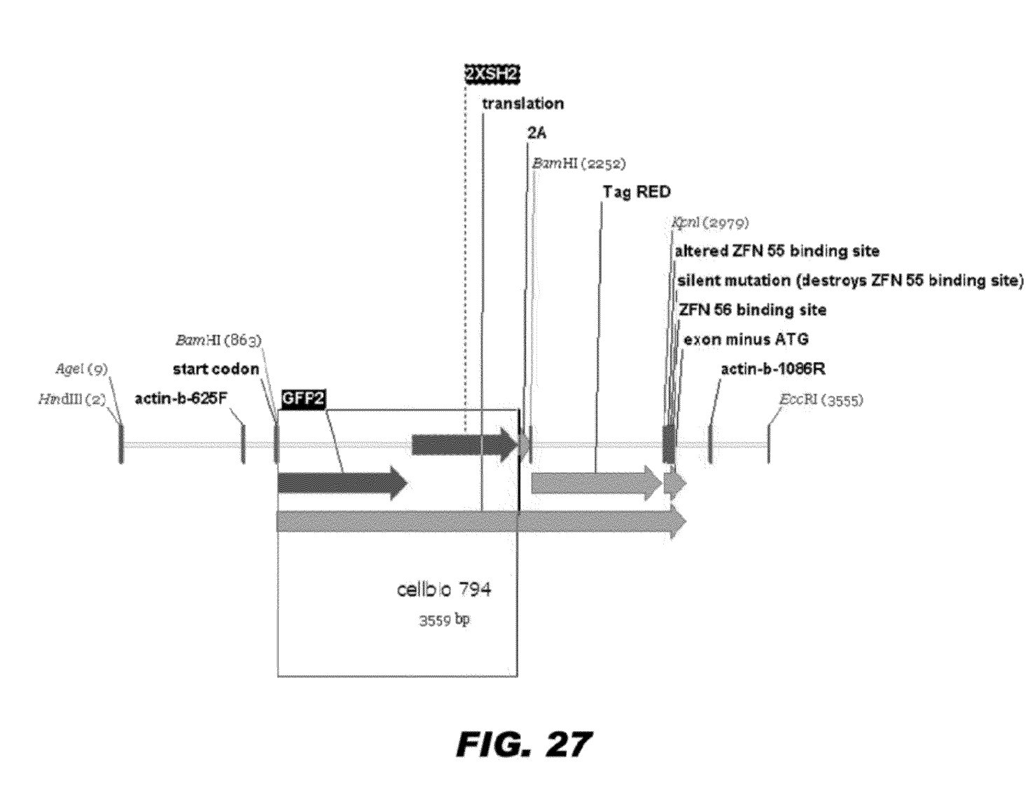

FIG. 27 presents the map of a donor plasmid comprising the SH2 biosensor sequence flanked by the ACTB sequences at the target region.

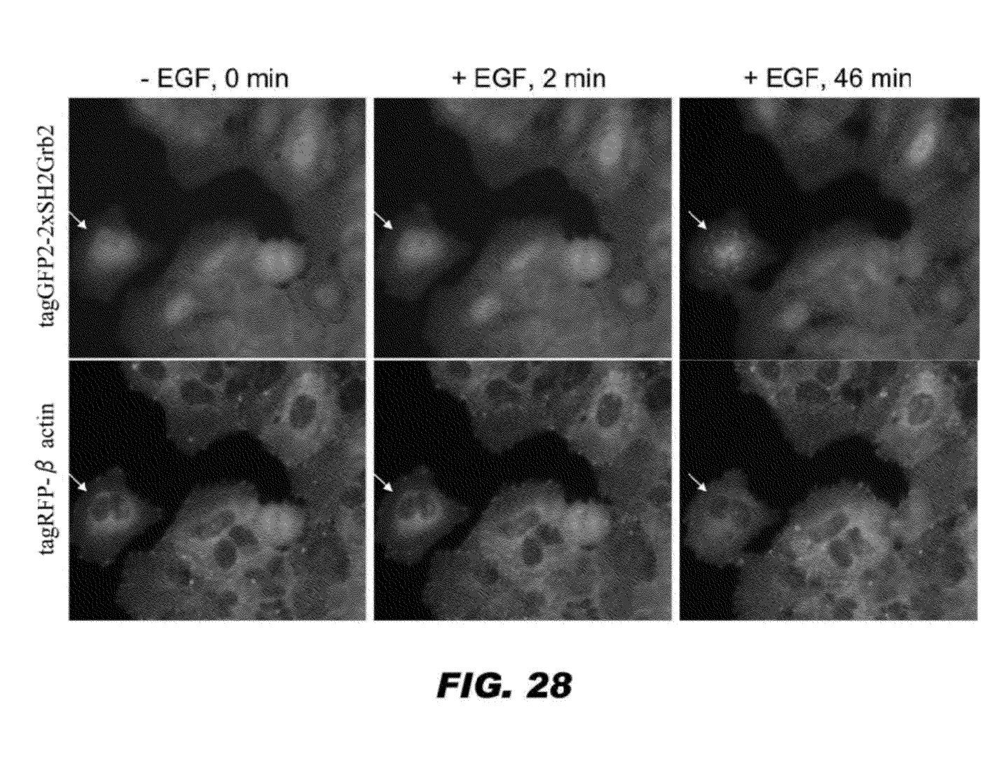

FIG. 28 depicts fluorescence microscopy images of individual isolated cell clones expressing GFP-2xSH2-Grb1-2A (upper panels) and RFP-β-actin (lower panels). Presented is a time course after exposure to 100 ng/mL of EGF.

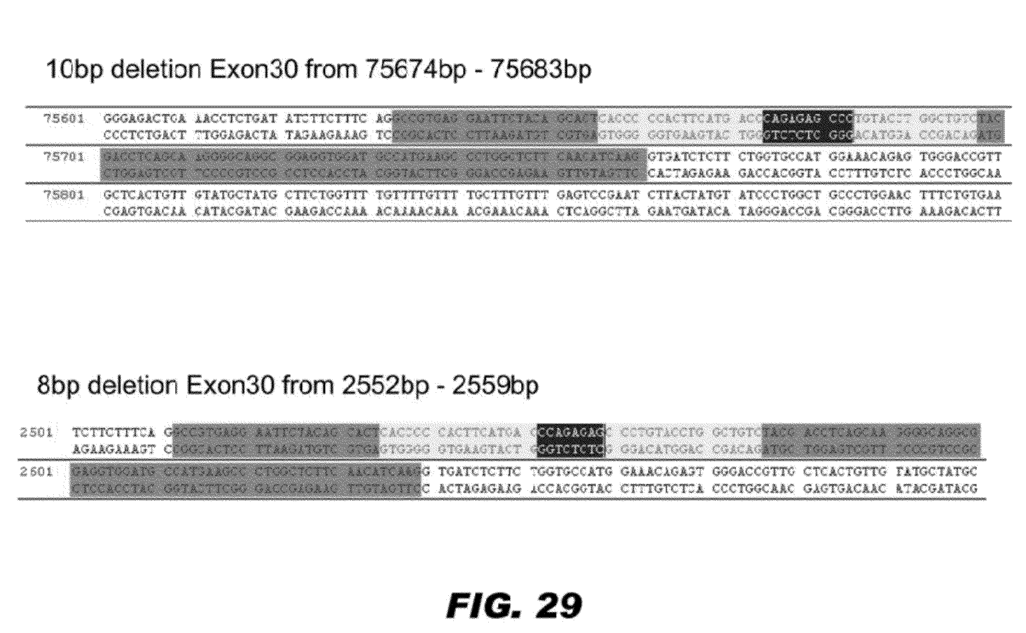

FIG. 29 presents the DNA sequences of two edited LRRK2 loci. The upper sequence (SEQ ID NO:92) has a 10 bp deletion in the target sequence of exon 30, and the lower sequence (SEQ ID NO:93) has a 8 bp deletion in the target sequence of exon 30. The exon is shown in green; the target site is presented in yellow, and the deletions are shown in dark blue.

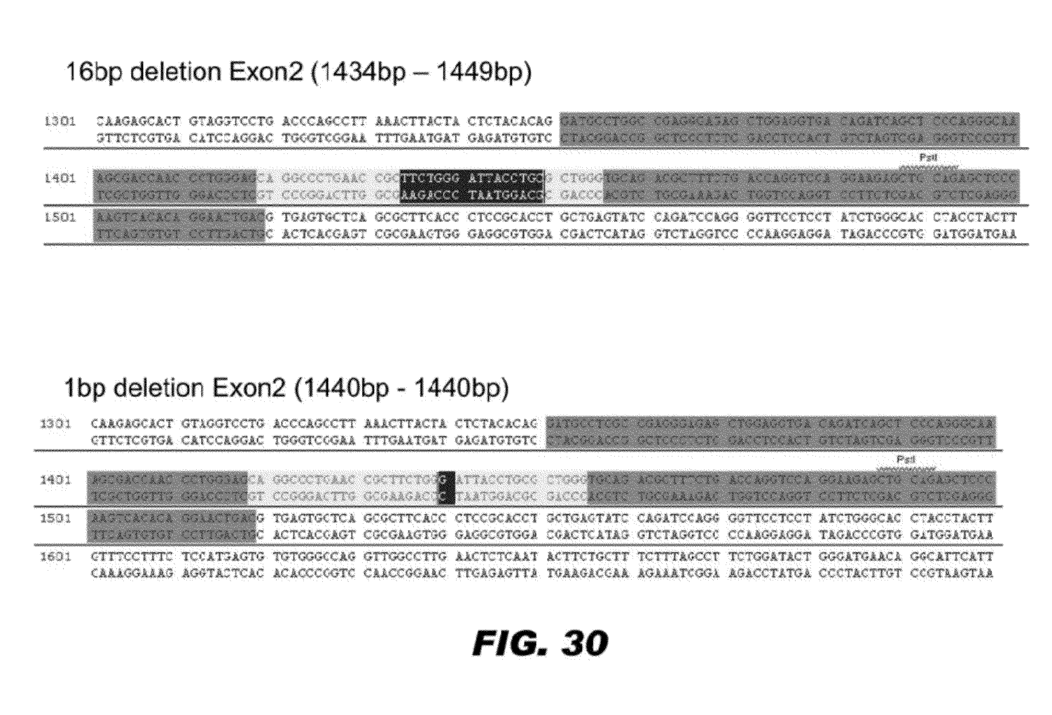

FIG. 30 presents the DNA sequences of two edited ApoE loci. The upper sequence (SEQ ID NO:114) has a 16 bp deletion in the target sequence of exon 2, and the lower sequence (SEQ ID NO:115) has a 1 bp deletion in the target sequence of exon 2. The exon sequence is shown in green; the target site is presented in yellow, and the deletions are shown in dark blue.

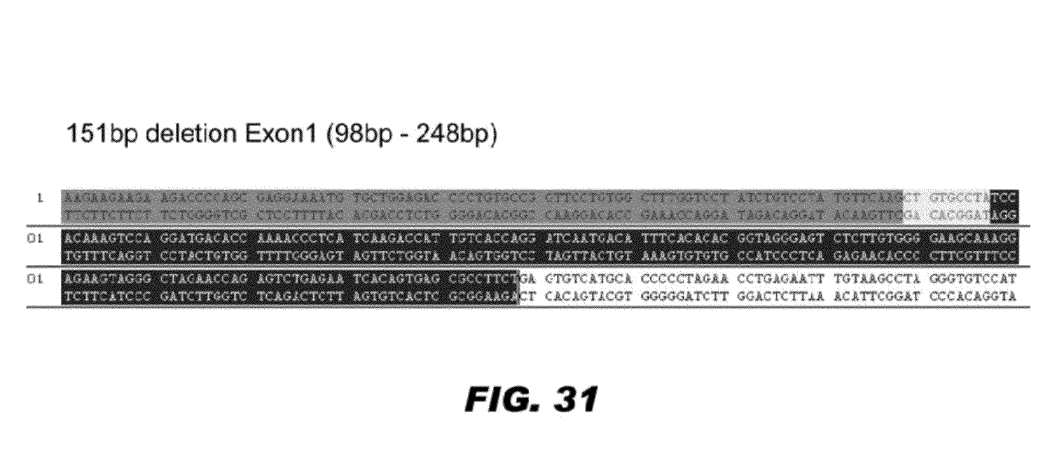

FIG. 31 shows the DNA sequence of an edited leptin locus. Presented is a region of the leptin locus (SEQ ID NO:116) in which 151 bp are deleted from exon 1 and intron 1. The exon is shown in green; the target site is presented in yellow, and the deletion is shown in dark blue.

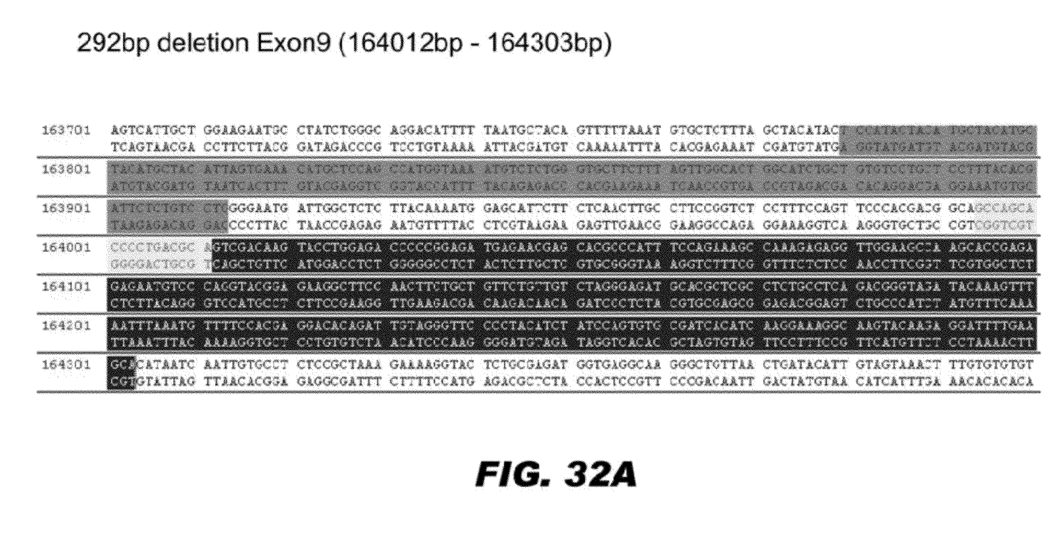

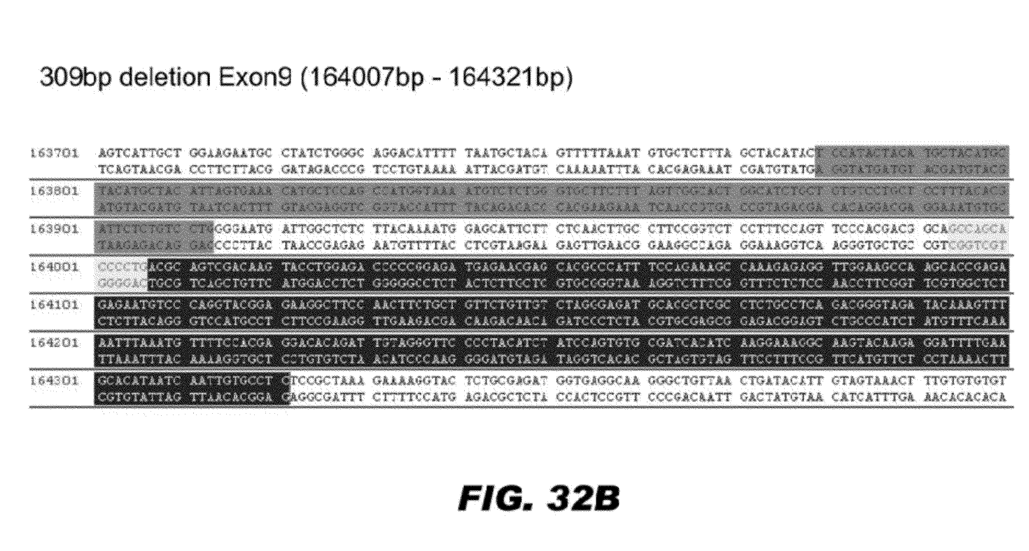

FIG. 32 presents the DNA sequences of edited APP loci in two animals. (A) Shows a region of the rat APP locus (SEQ ID NO:127) in which 292 bp is deleted from exon 9. (B) Presents a region of the rat APP locus (SEQ ID NO:128) in which there is a 309 bp deletion in exon 9. The exon is shown in green; the target site is presented in yellow, and the deletion is shown in dark blue.

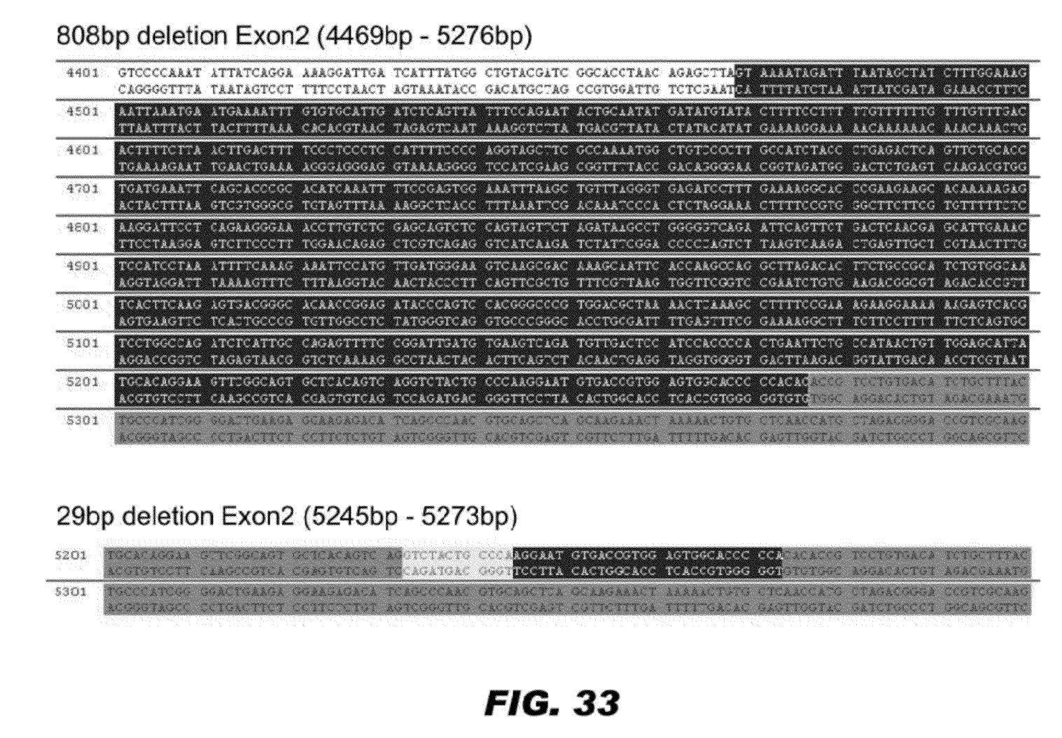

FIG. 33 presents the DNA sequences of edited Rag1 loci in two animals. The upper sequence (SEQ ID NO:131) has a 808 bp deletion in exon 2, and the lower sequence (SEQ ID NO:132) has a 29 bp deletion in exon 2. The exon sequence is shown in green; the target site is presented in yellow, and the deletions are shown in dark blue.

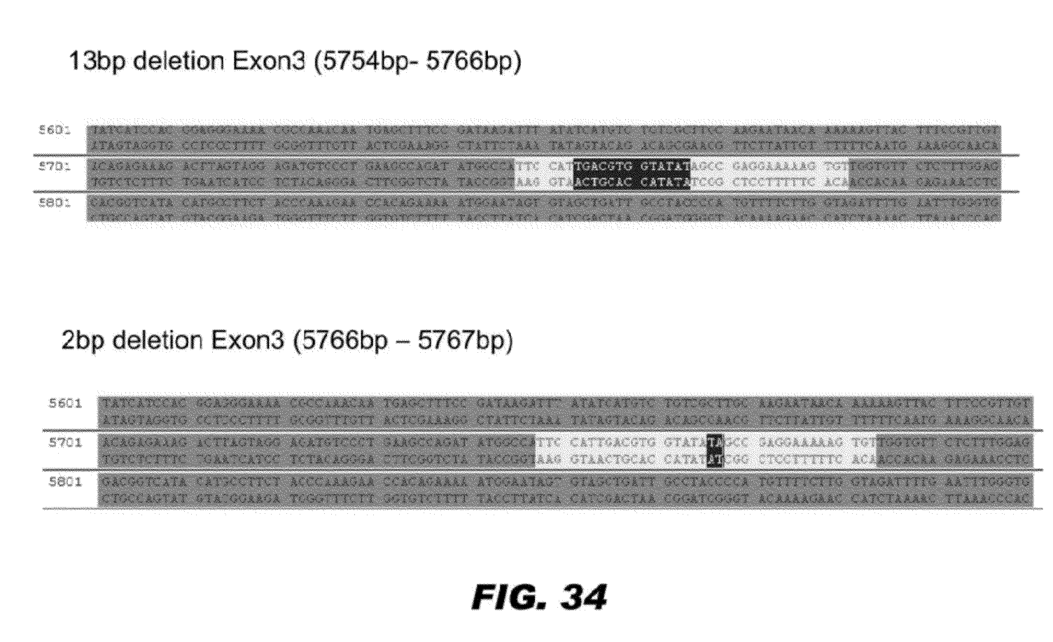

FIG. 34 presents the DNA sequences of edited Rag2 loci in two animals. The upper sequence (SEQ ID NO:133) has a 13 bp deletion in the target sequence in exon 3, and the lower sequence (SEQ ID NO:134) has a 2 bp deletion in the target sequence in exon 2. The exon sequence is shown in green; the target site is presented in yellow, and the deletions are shown in dark blue.

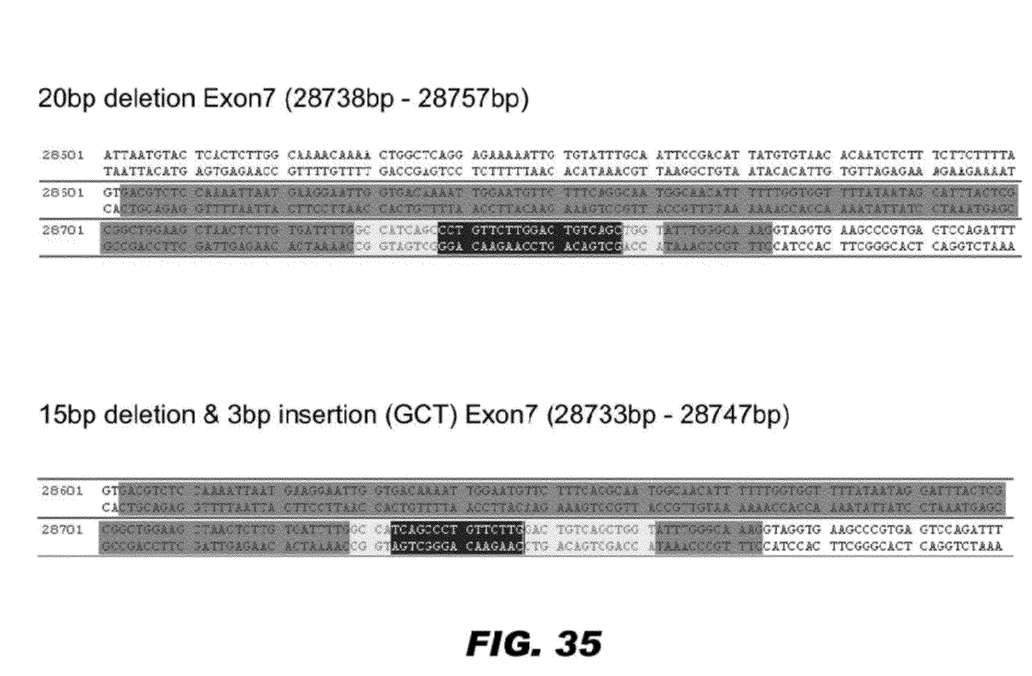

FIG. 35 presents the DNA sequences of edited Mdr1a loci in two animals. The upper sequence (SEQ ID NO:157) has a 20 bp deletion in exon 7, and the lower sequence (SEQ ID NO:158) has a 15 bp deletion and a 3 bp insertion (GCT) in exon 7. The exon sequence is shown in green; the target sequence is presented in yellow, and the deletions are shown in dark blue.

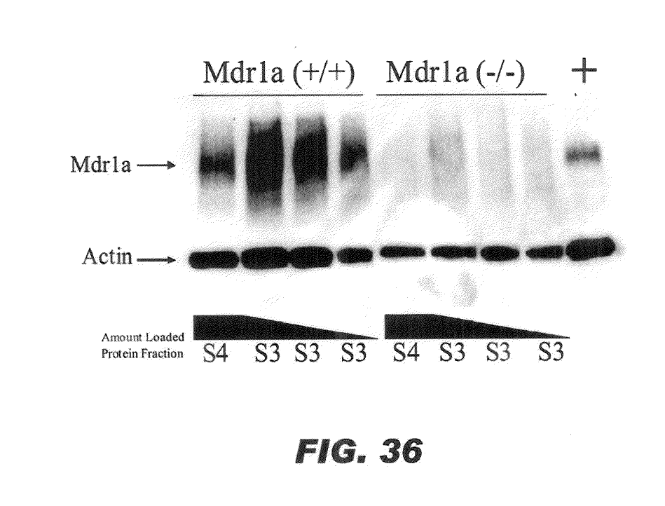

FIG. 36 illustrates knockout of the Mdr1a gene in rat. Presented is a Western blot of varying amounts of a colon lysate from an Mdr1a knockout rat and a control cell lysate. The relative locations Mdr1a protein and actin protein are indicated to the left of the image

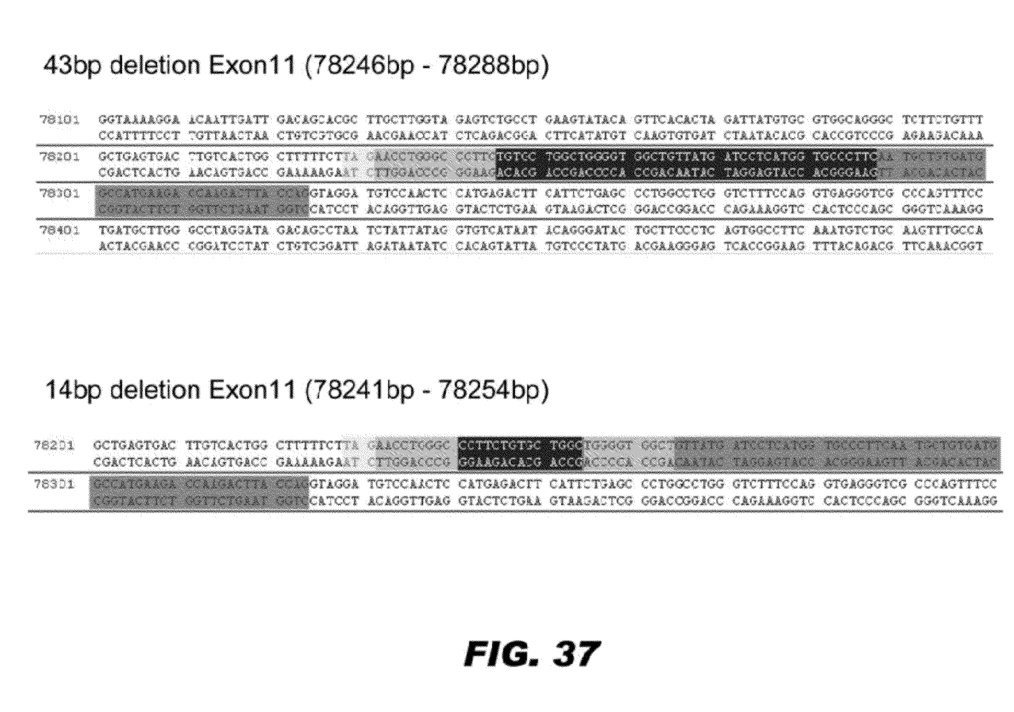

FIG. 37 presents the DNA sequences of edited Mrp1 loci in two animals. The upper sequence (SEQ ID NO:159) has a 43 bp deletion in exon 11, and the lower sequence (SEQ ID NO:160) has a 14 bp deletion in exon 11. The exon sequence is shown in green; the target sequence is presented in yellow, the deletions are shown in dark blue; and overlap between the target sequence and the exon is shown in grey.

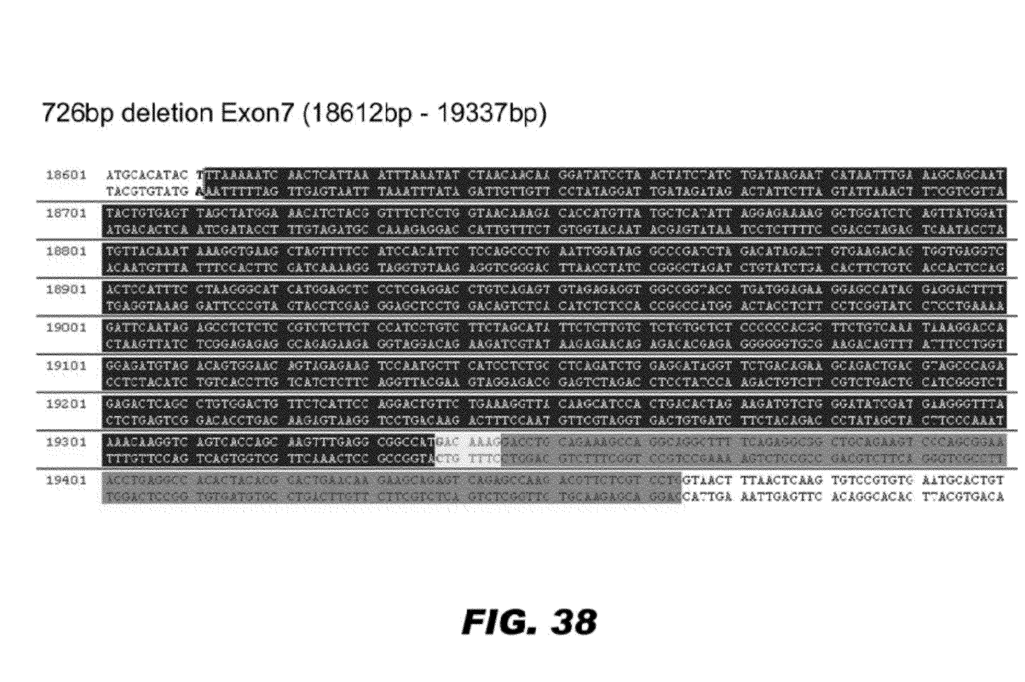

FIG. 38 shows the DNA sequence of an edited Mrp2 locus. The sequence (SEQ ID NO:161) has a 726 bp deletion in exon 7. The exon is shown in green; the target sequence is presented in yellow, and the deletion is shown in dark blue.

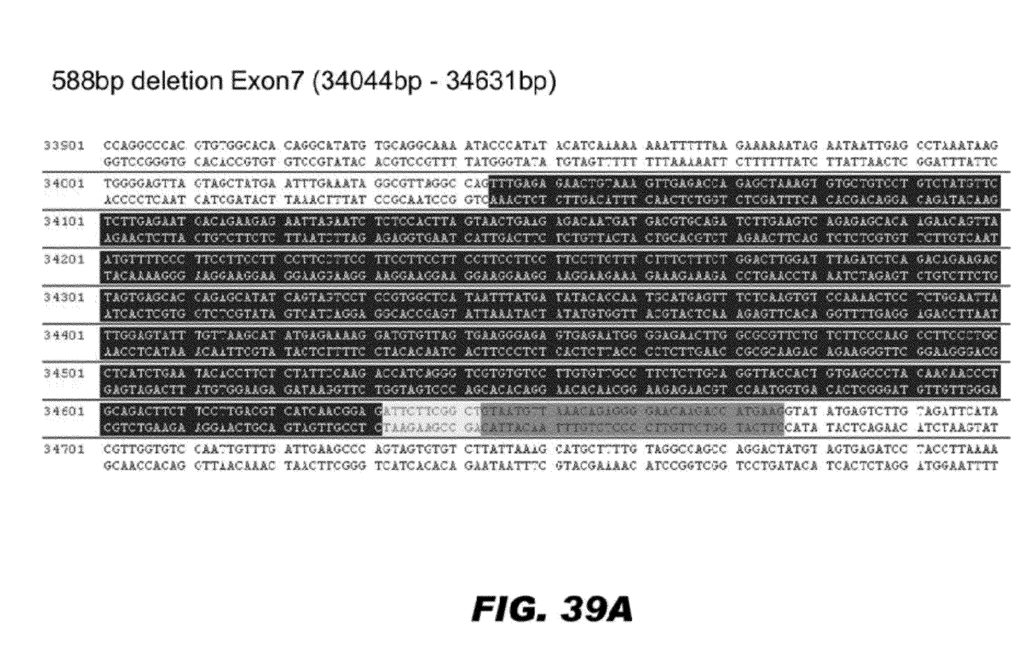

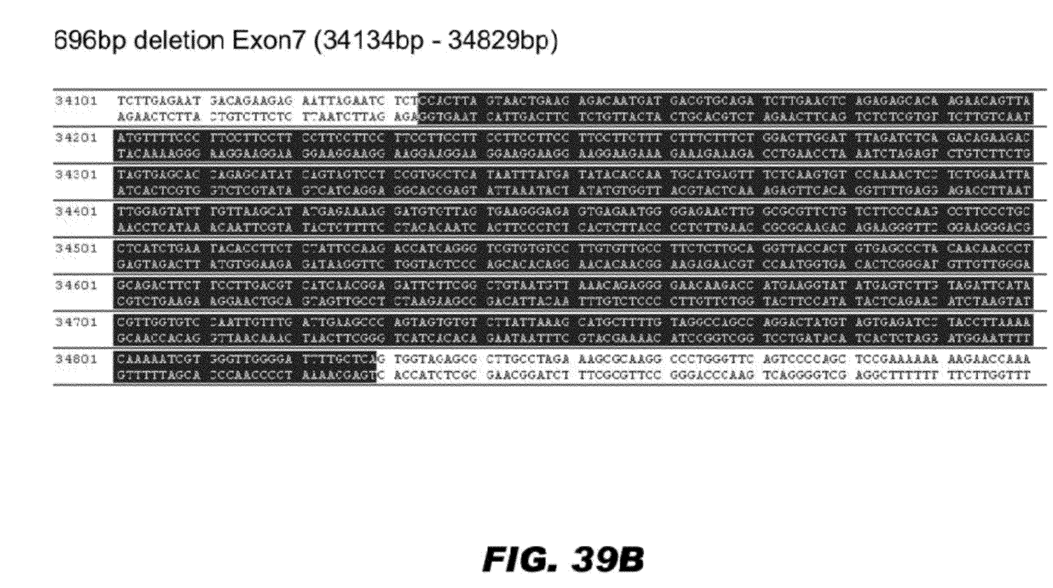

FIG. 39 presents the DNA sequences of edited BCRP loci in two animals. (A) Shows a region of the rat BCRP locus (SEQ ID NO:162) comprising a 588 bp deletion in exon 7. (B) Presents a region of the rat BCRP locus (SEQ ID NO:163) comprising a 696 bp deletion in exon 7. The exon sequence is shown in green; the target site is presented in yellow, and the deletions are shown in dark blue.

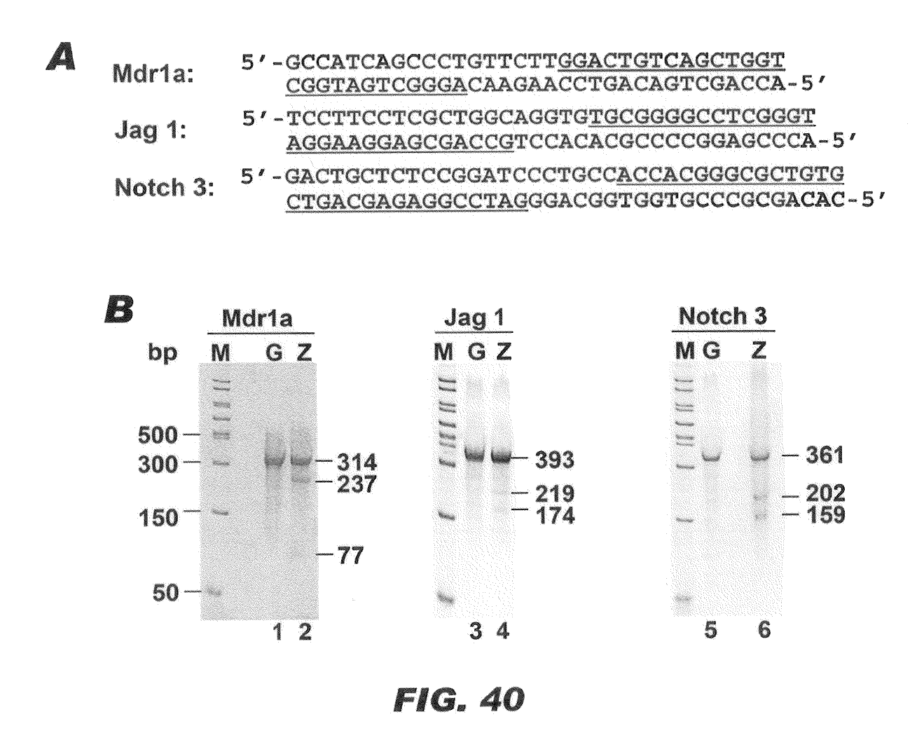

FIG. 40 presents target sites and ZFN validation of Mdr1a, and two additional genes, Jag1, and Notch3. (A) shows ZFN target sequences. The ZFN binding sites are underlined. (B) shows results of a mutation detection assay in NIH 3T3 cells to validate the ZFN mRNA activity. Each ZFN mRNA pair was cotransfected into NIH 3T3 cells. Transfected cells were harvested 24 h later. Genomic DNA was analyzed with the mutation detection assay to detect NHEJ products, indicative of ZFN activity. M, PCR marker; G (lanes 1, 3, and 5): GFP transfected control; Z (lanes 2, 4, and 6), ZFN transfected samples. Uncut and cleaved bands are marked with respective sizes in base pairs.

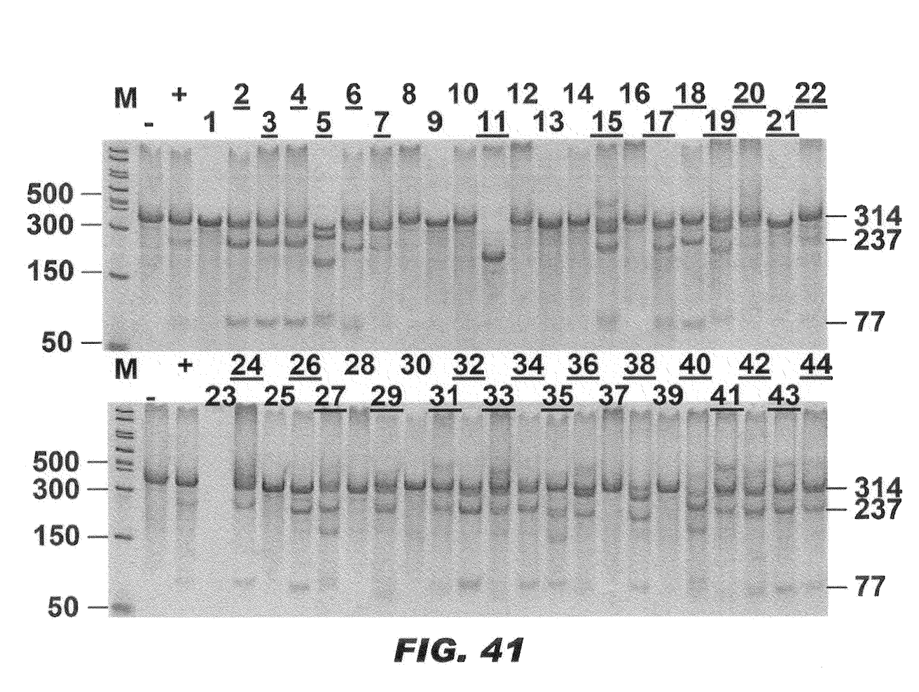

FIG. 41 presents identification of genetically engineered Mdr1a founders using a mutation detection assay. Uncut and cleaved bands are marked with respective sizes in base pairs. Cleaved bands indicate a mutation is present at the target site. M, PCR marker. 1-44, 44 pups born from injected eggs. The numbers of founders are underlined.

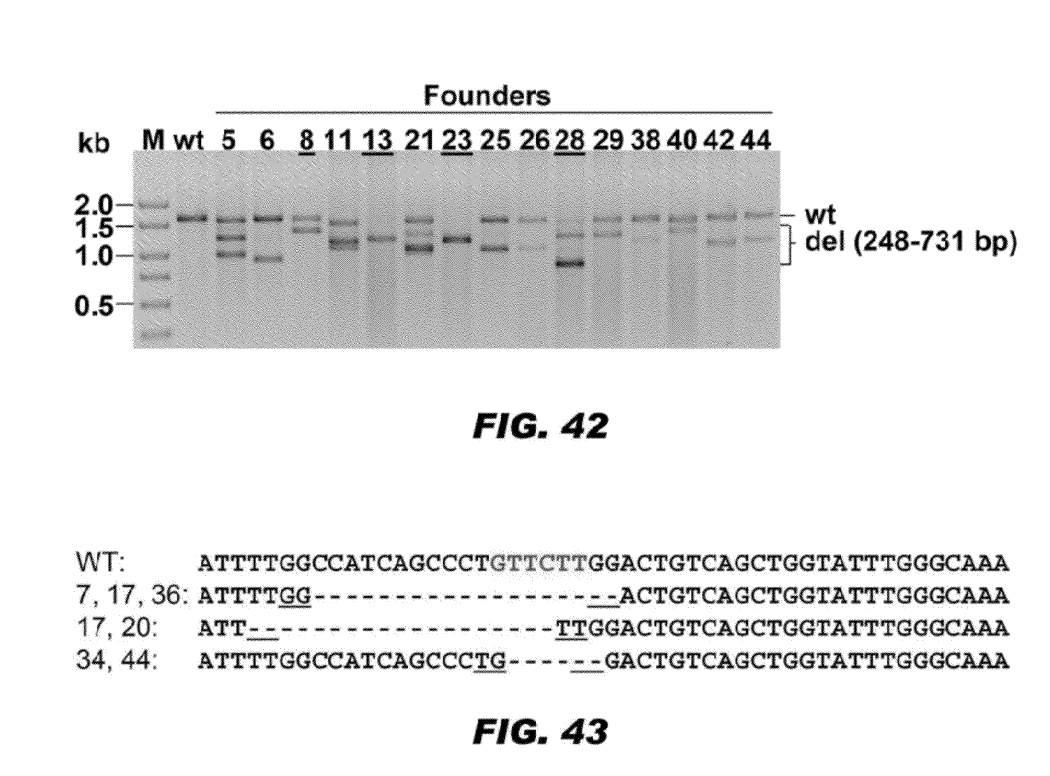

FIG. 42 presents amplification of large deletions in Mdr1a founders. PCR products were amplified using primers located 800 bp upstream and downstream of the ZFN target site. Bands significantly smaller than the 1.6 kb wild-type band indicate large deletions in the target locus. Four founders that were not identified in FIG. 7 are underlined.

FIG. 43 presents the results of a mutation detection assay at the Mdr1b site in 44 Mdr1a ZFN injected pups. M, PCR marker; WT, toe DNA from FVB/N mice that were not injected with Mdr1a ZFNs; 3T3, NIH 3T3 cells transfected with Mdr1a ZFNs as a control.

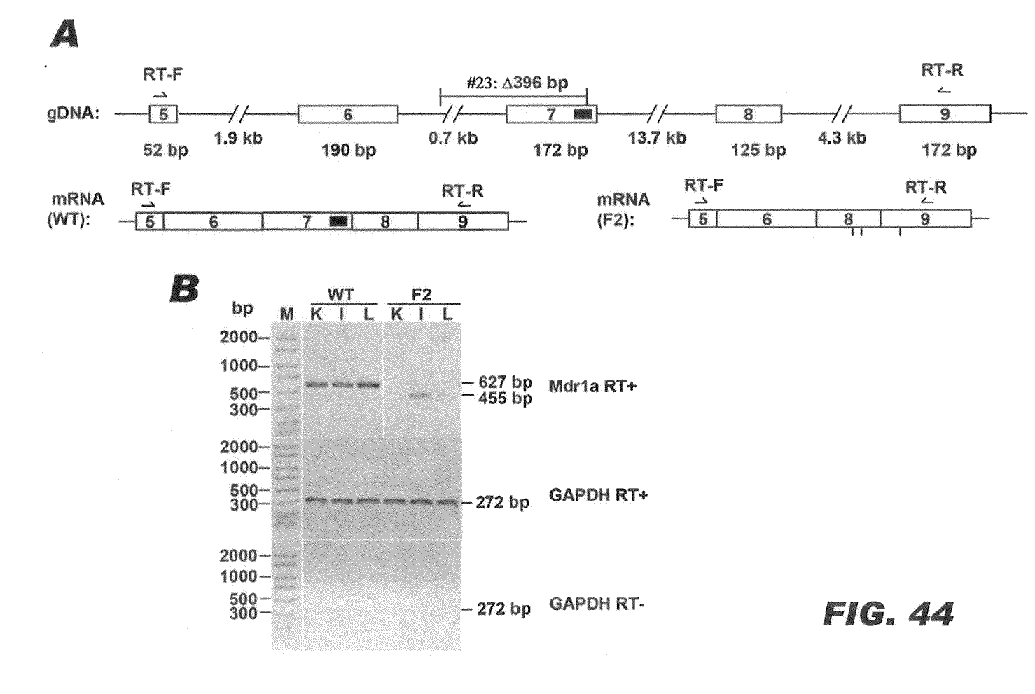

FIG. 44 presents detection of Mdr1a expression by using RT-PCR in Mdr1a−/− mice. (A) is a schematic illustration of Mdr1a genomic and mRNA structures around the target site. Exons are represented by open rectangles with respective numbers. The size of each exon in base pairs is labeled directly underneath. Intron sequences are represented by broken bars with size in base pairs underneath. The ZFN target site in exon 7 is marked with a solid rectangle. The position of the 396 bp deletion in founder #23 is labeled above intron 6 and exon 7. RT-F and RT-R are the primers used in RT-PCR, located in exons 5 and 9, respectively. In the RT reaction, 40 ng of total RNA was used as template. Normalization of the input RNA is confirmed by GAPDH amplification with or without RT.

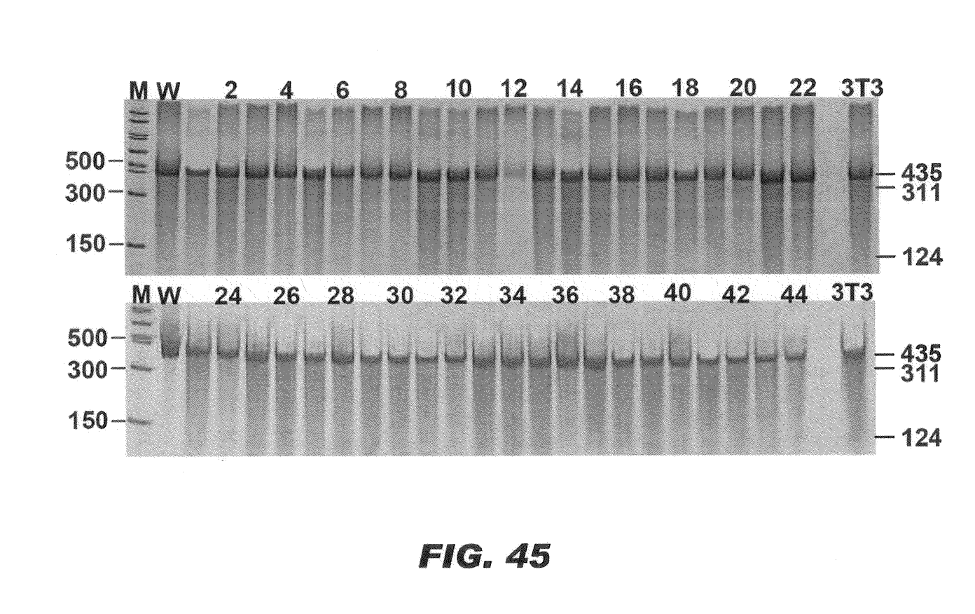

FIG. 45 presents the results of band isolation following isolation and purification of the species at the wild-type size in the Mdr1a−/− samples, and then use as a template in a nested PCR.

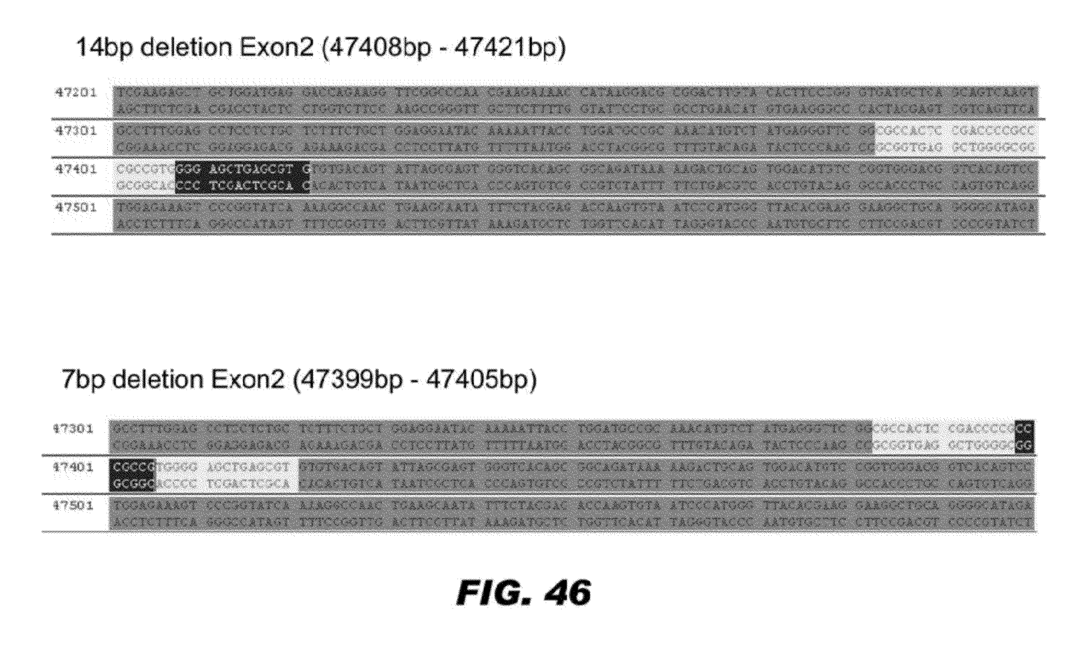

FIG. 46 shows the DNA sequences of edited BDNF loci in two animals. The upper sequence (SEQ ID NO:211) has a 14 bp deletion in the target sequence in exon 2, and the lower sequence (SEQ ID NO:212) has a 7 bp deletion in the target sequence in exon 2. The exon is shown in green; the target site is presented in yellow, and the deletions are shown in dark blue.

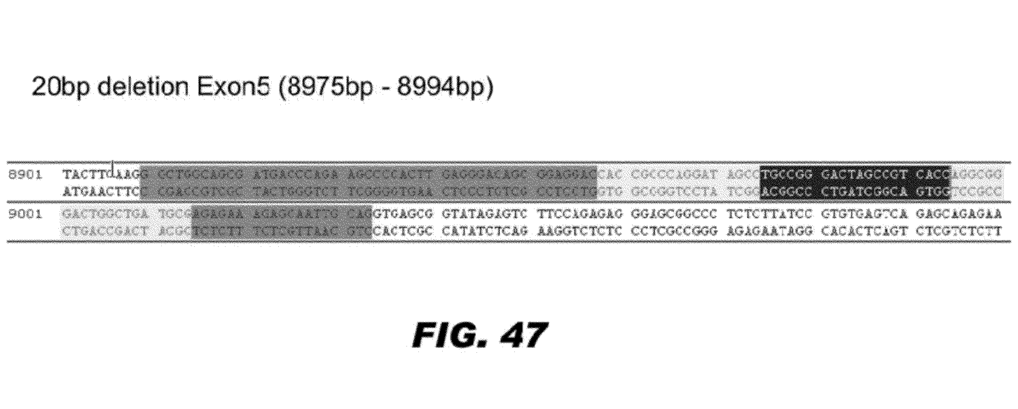

FIG. 47 presents the DNA sequence of an edited DISC1 locus. Presented is a region of the rat DISC1 (SEQ ID NO:225) in which there is a 20 bp deletion in the target sequence in exon 5. The exon is shown in green; the target site is presented in yellow, and the deletion is shown in dark blue.

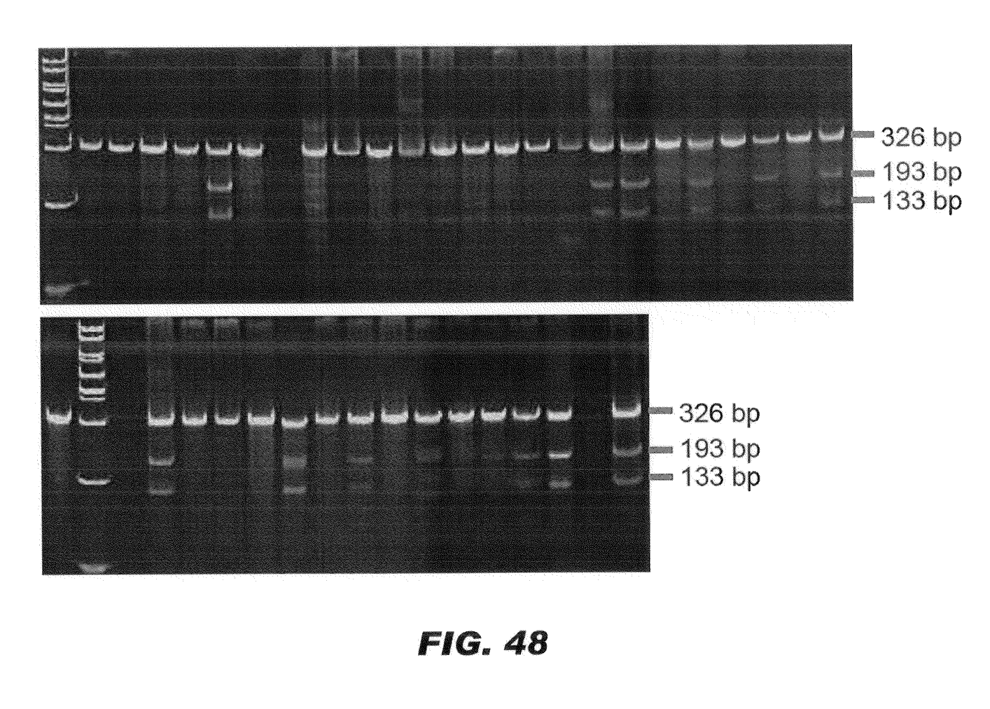

FIG. 48 illustrates editing of the p53 locus in rats. Presented is a Cel-1 assay in which the presence of cleavage products indicated editing of the p53 gene.

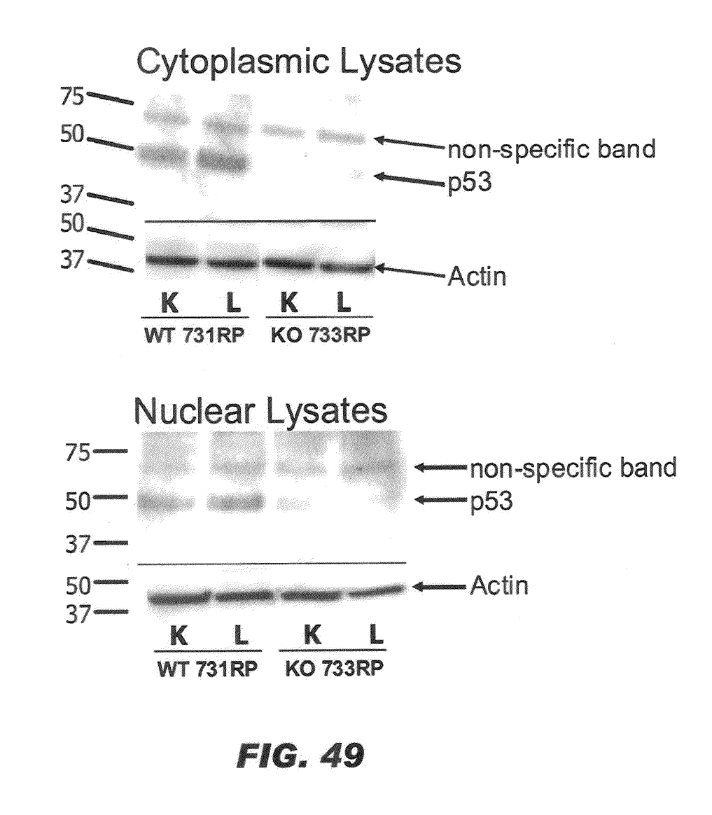

FIG. 49 illustrates knockout of the p53 gene in rats. Presented are Western blots of cytoplasmic and nuclear lysates of kidney (K) and liver (L) samples from wild-type (WT 731RP) and p53 knockout (KO 733RP) animals. The relative locations p53 protein and actin protein are indicated to the right of each image.

### DETAILED DESCRIPTION OF THE INVENTION

The present disclosure provides a method for creating a genetically modified animal or animal cell comprising at least one edited chromosomal sequence. The edited chromosomal sequence may be (1) inactivated, (2) modified, or (3) comprise an integrated sequence. An inactivated chromosomal sequence is altered such that a functional protein is not made or a control sequence no longer functions the same as a wild-type control sequence. Thus, a genetically modified animal comprising an inactivated chromosomal sequence may be termed a “knock-out” or a “conditional knock-out.” Similarly, a genetically modified animal comprising an integrated sequence may be termed a “knock-in” or a “conditional knock-in.” As detailed below, a knock-in animal may be a humanized animal. Furthermore, a genetically modified animal comprising a modified chromosomal sequence may comprise a targeted point mutation(s) or other modification such that an altered protein product is produced. A chromosomal sequence generally is edited using a zinc finger nuclease-mediated process. Briefly, the process comprises introducing into a cell at least one nucleic acid encoding a targeted zinc finger nuclease and, optionally, at least one accessory polynucleotide. The method further comprises incubating the cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. In an exemplary embodiment, the cell is an embryo. The method of editing chromosomal sequences using targeted zinc finger nuclease technology as described herein is rapid, precise, and highly efficient.

Additionally, the invention encompasses an animal or a cell comprising at least one edited chromosomal sequence. A method of the invention, an animal of the invention, a cell of the invention, and applications thereof are described in more detail below.

### I. Method for Chromosomal Editing

One aspect of the present invention encompasses a method for chromosomal editing. As used herein, “chromosomal editing” refers to editing a chromosomal sequence such that the sequence is (1) inactivated, (2) modified, or (3) comprises an integrated sequence. Generally speaking, a method for editing a chromosomal sequence comprises: (a) introducing into a cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration, the sequence flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the cell to allow expression of the zinc finger nuclease such that the zinc finger nuclease introduces a double-stranded break into the chromosomal sequence, and wherein the double-stranded break is repaired by (i) a non-homologous end-joining repair process such that a mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method of editing a chromosomal sequence are described in more detail below.

### (a) Nucleic Acid Encoding a Zinc Finger Nuclease

The method comprises, in part, introducing into a cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

Generally speaking, a zinc finger nuclease of the invention, once introduced into a cell, creates a double-stranded break in the chromosomal sequence. The double-stranded break may be repaired, in certain embodiments, by a non-homologous end-joining repair process of the cell, such that a mutation is introduced into the chromosomal sequence. In other embodiments, as described below, a homology-directed repair process is used to edit the chromosomal sequence.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publicly available web-based tools for identifying potential target sites in DNA sequences and designing zinc finger binding domains may be found at www.zincfingertools.org and bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger DNA binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain.

The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:I538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

(iii) Exemplary Zinc Finger Nuclease

Provided herein are non-limiting examples of zinc finger nucleases that recognize and bind target sequences found in various animal chromosomal sequences. For instance, a zinc finger nuclease of the invention may have an amino acid sequence that is at least 80% identical to a sequence chosen from a zinc finger nuclease having a SEQ ID NO chosen from 53, 54, 57-62, 69-76, 104-113, 123-126, 147-156, 201-210, 219-222, 223-224, 230-233, 240-243. In other embodiments, the sequence identity may be about 81, 82, 83, 84, 85, 86, 87, 88, 89, 90, 91, 92, 93, 94, 95, 96, 97, 98, 99, or 100%.

Moreover, the zinc finger nucleases encoded by a SEQ ID NO chosen from 53, 54, 57-62, 69-76, 104-113, 123-126, 147-156, 201-210, 219-222, 223-224, 230-233, 240-243 may recognize and bind a chromosomal sequence having at least about 80, 81, 82, 83, 84, 85, 86, 87, 88, 89, 90, 91, 92, 93, 94, 95, 96, 97, 98, 99, or 100% sequence identity to a chromosomal SEQ ID NO 55, 56, 63-68, 77-84, 86-91, 94-103, 117-122, 129, 130, 135, 136, 137, 138, 139-146, 164-173, 213-218, 226-229, 234, 235, 236, 237, 238, 239.

(iv) Additional Methods for Targeted Cleavage

Any nuclease having a target site in a chromosome may be used in the methods disclosed herein. For example, homing endonucleases and meganucleases have very long recognition sequences, some of which are likely to be present, on a statistical basis, once in a human-sized genome. Any such nuclease having a unique target site in a genome may be used instead of, or in addition to, a zinc finger nuclease, for targeted cleavage of a chromosome.

Non-limiting examples of homing endonucleases include I-SceI, I-CeuI, PI-PspI, PI-Sce, I-SceIV, I-CsmI, I-Pant, I-SceII, I-PpoI, I-SceIII, I-CreI, I-TevI, I-TevII and I-TevIII. The recognition sequences of these enzymes are known in the art. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

Although the cleavage specificity of most homing endonucleases is not absolute with respect to their recognition sites, the sites are of sufficient length that a single cleavage event per mammalian-sized genome may be obtained by expressing a homing endonuclease in a cell containing a single copy of its recognition site. It has also been reported that the specificity of homing endonucleases and meganucleases may be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66.

### (b) Optional Exchange Polynucleotide

A method for editing chromosomal sequences may further comprise introducing into a cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may be at least about 80, 81, 82, 83, 84, 85, 86, 87, 88, 89, 90, 91, 92, 93, 94, 95, 96, 97, 98, or 99% identical to a region of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein may be produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 25 bp to about 10,000 bp in length. In various embodiments, the sequence in the exchange polynucleotide may be about 50, 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (c) Optional Donor Polynucleotide

A method for editing chromosomal sequences may alternatively comprise introducing at least one donor polynucleotide comprising a sequence for integration into a cell. A donor polynucleotide comprises at least three components: the sequence to be integrated that is flanked by an upstream sequence and a downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide may be a DNA plasmid.

The donor polynucleotide comprises a sequence for integration. The sequence for integration may be a sequence endogenous to the animal or cell or it may be an exogenous sequence. The sequence for integration may encode a protein or a non-coding RNA (e.g., a microRNA). Thus, the sequence for integration may be operably linked to an appropriate control sequence or sequences. Alternatively, the sequence for integration may provide a regulatory function. Accordingly, the size of the sequence for integration can and will vary. In general, the sequence for integration may range from about one nucleotide to several million nucleotides.

The donor polynucleotide also comprises upstream and downstream sequence flanking the sequence to be integrated. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 20 bp to about 2500 bp. In various embodiments, an upstream or downstream sequence may comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for editing a chromosomal sequence by integrating a sequence, the double stranded break introduced into the chromosomal sequence by the zinc finger nuclease is repaired, via homologous recombination with the donor polynucleotide, such that the sequence is integrated into the chromosome. The presence of a double-stranded break facilitates integration of the sequence. A donor polynucleotide may be physically integrated or, alternatively, the donor polynucleotide may be used as a template for repair of the break, resulting in the introduction of the sequence as well as all or part of the upstream and downstream sequences of the donor polynucleotide into the chromosome. Thus, the endogenous chromosomal sequence may be converted to the sequence of the donor polynucleotide.

(d) Introducing Nucleic Acid into a Cell

To mediate zinc finger nuclease genome editing, at least one nucleic acid molecule encoding a zinc finger nuclease and, optionally, at least one exchange polynucleotide or at least one donor polynucleotide is introduced into a cell. As used herein, the term “cell” encompasses any animal cell that comprises a chromosomal sequence. In some embodiments, the term “cell” may refer to an embryo. In certain exemplary embodiments, the embryo is a fertilized one-cell stage embryo. In other exemplary embodiments, the embryo may be an embryo of any stage.

Suitable methods of introducing the nucleic acids to the embryo or cell may include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and an exchange (or donor) polynucleotide are introduced into an embryo or cell, the ratio of exchange (or donor) polynucleotide to nucleic acid encoding a zinc finger nuclease may range from about 1:10 to about 10:1. In various embodiments, the ratio of exchange (or donor) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one exchange (or donor) polynucleotide is introduced into an embryo or cell, the nucleic acids may be introduced simultaneously or sequentially. For example, nucleic acids encoding the zinc finger nucleases, each specific for a distinct recognition sequence, as well as the optional exchange (or donor) polynucleotides, may be introduced at the same time. Alternatively, each nucleic acid encoding a zinc finger nuclease, as well as the optional exchange (or donor) polynucleotides, may be introduced sequentially.

In one embodiment, at least one nucleic acid molecule encoding a zinc finger nuclease is introduced into a cell. In another embodiment, at least 2, 3, 4, 5, or more than 5 nucleic acid molecules encoding a zinc finger nuclease are introduced into a cell. In each of the above embodiments, one or more corresponding donor or exchange polynucleotides may also be introduced into the cell, in a ratio from about 1:10 to about 10:1 donor or exchange polynucleotides to zinc finger nuclease nucleic acids, as described above.

### (e) Culturing the Cell

A method for editing a chromosomal sequence using a zinc finger nuclease-mediated process as described herein further comprises culturing the cell comprising the introduced nucleic acid(s) to allow expression of the at least one zinc finger nuclease.

Cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type or cell species. Routine optimization may be used, in all cases, to determine the best techniques for a particular cell type.

In one embodiment where the cell is an embryo, the embryo may be cultured in vitro (e.g., in cell culture). Typically, the embryo is cultured for a short period of time at an appropriate temperature and in appropriate media with the necessary O2/CO2 ratio to allow the expression of the zinc finger nuclease. A skilled artisan will appreciate that culture conditions can and will vary depending on the embryo species. Routine optimization may be used, in all cases, to determine the best culture conditions for a particular species of embryo. In some cases, a cell line may be derived from an in vitro-cultured embryo (e.g., an embryonic stem cell line).

Preferably, the embryo will be cultured in vivo by transferring the embryo into the uterus of a female host. Generally speaking, the female host is from the same or a similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal generally will comprise the disrupted chromosomal sequence(s) in every cell of its body.

Upon expression of the at least one zinc finger nuclease in a cell, the chromosomal sequence of the cell may be edited. In cases in which the cell comprises an expressed zinc finger nuclease but no exchange (or donor) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, or insertion resulting in a missense or nonsense mutation may be introduced in the chromosomal sequence such that the sequence is inactivated.

In cases in which the embryo or cell comprises an expressed zinc finger nuclease as well as an exchange (or donor) polynucleotide, the zinc finger nuclease recognizes, binds, and cleaves the target sequence in the chromosome. The double-stranded break introduced by the zinc finger nuclease is repaired, via homologous recombination with the exchange (or donor) polynucleotide, such that a portion of the chromosomal sequence is converted to the sequence in the exchange polynucleotide or the sequence in the donor polynucleotide is integrated into the chromosomal sequence. As a consequence, the chromosomal sequence is edited.

The genetically modified animals disclosed herein may be crossbred to create animals comprising more than one edited chromosomal sequence or to create animals that are homozygous for one or more edited chromosomal sequences. Those of skill in the art will appreciate that many combinations are possible. Moreover, the genetically modified animals disclosed herein may be crossed with other animals to combine the edited chromosomal sequence with other genetic backgrounds. By way of non-limiting example, suitable genetic backgrounds include wild-type, natural mutations giving rise to known phenotypes, targeted chromosomal integration, non-targeted integrations, etc.

### (f) Types of Chromosomal Edits

As stated above, a method of the invention may be used to (1) inactivate a chromosomal sequence, (2) modify a chromosomal sequence, or (3) integrate a sequence into a chromosome. Each of these is discussed in more detail below.

i. Inactivate a Sequence

In one embodiment, an edited chromosomal sequence may be inactivated such that the sequence is not transcribed, the coded protein is not produced, or the sequence does not function as the wild-type sequence does. For example, a protein coding sequence may be inactivated such that the protein is not produced. Alternatively, a microRNA coding sequence may be inactivated such that the microRNA is not produced. Furthermore, a control sequence may be inactivated such that it no longer functions as a control sequence. As used herein, “control sequence” refers to any nucleic acid sequence that effects the transcription, translation, or accessibility of a nucleic acid sequence. By way of non-limiting example, a promoter, a transcription terminator, and an enhancer are control sequences. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). In some embodiments, a chromosomal sequence that is inactivated may be termed a “knock-out.” In an interation of the invention, a “knock-out” animal created by a method of the invention does not comprise any exogenous sequence.

ii. Modify a Sequence

In another embodiment, an edited chromosomal sequence may be modified such that it codes for an altered gene product or the function of the sequence is altered. A chromosomal sequence encoding a protein may be modified to include at least one changed nucleotide such that the codon comprising the changed nucleotide codes for a different amino acid. The resultant protein, therefore, comprises at least one amino acid change. Moreover, a protein coding sequence may be modified by insertions or deletions such that the reading from of the sequence is not altered and a modified protein is produced. In such embodiments, the modified sequence may result in a phenotype change.

Alternatively, a chromosomal sequence that functions as a control sequence may be modified. For instance, a promoter may be modified such that it is always active or is regulated by an exogenous signal.

In yet another embodiment, at least one chromosomal sequence encoding a protein of interest may be edited such that the expression pattern of the protein is altered. For example, regulatory regions controlling the expression of the protein, such as a promoter or transcription factor binding site, may be altered such that the protein of interest is over-produced, or the tissue-specific or temporal expression of the protein is altered, or a combination thereof.

iii. Integrate a Sequence

In yet another embodiment, an edited chromosomal sequence may comprise an integrated sequence. Such a sequence may encode an endogenous protein, an exogenous or heterologous protein, a wild-type protein, a modified protein, a fusion protein, a microRNA, or the like. An integrated protein coding sequence may be linked to a reporter sequence (the reporter sequence may be linked 5′ or 3′ to the protein coding sequence). An integrated protein coding sequence may also be placed under control of an endogenous promoter, may be operably linked to an exogenous promoter, or may be fused in-frame with an endogenous protein coding sequence. Additionally, the integrated sequence may function as a control element. Accordingly, the integrated sequence may be endogenous or exogenous to the cell. An animal or cell comprising such an integrated sequence may be termed “knock-in.” In one iteration of the above embodiments, it should be understood that no selectable marker is present.

In certain embodiments, a sequence may be integrated to alter the expression pattern of a protein of interest. For instance, a conditional knock-out system may be created.

### A. Conditional Mutations

In certain embodiments, a sequence may be edited to alter the expression pattern of a protein of interest. For instance, a conditional knock-out system may be created.

As used herein, a “conditional knock-out” system is a model where the expression of a nucleic acid molecule is disrupted in a particular organ, tissue, or cell type, as opposed to the entire animal, and/or in a temporally controlled manner. A conditional knock-out allows, for example, the study of a gene function even when global disruption of the gene is lethal.

A non-limiting example of a conditional knock-out system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified cell is generated with lox sites flanking a chromosomal sequence of interest. A genetically modified animal comprising a cell with the lox-flanked chromosomal sequence of interest may then be crossed with another genetically modified animal expressing Cre recombinase in one or more cells. Progeny animals comprising one or more cells comprising a lox-flanked chromosomal sequence and one or more cells comprising a Cre recombinase are then produced. In the cells that comprise both a lox-flanked chromosomal sequence and a Cre recombinase, the lox-flanked chromosomal sequence encoding a protein of interest is recombined, leading to deletion or inversion of the chromosomal sequence encoding the protein of interest. Expression of Cre recombinase may be temporally and conditionally regulated to effect temporally and conditionally regulated recombination of the chromosomal sequence encoding the protein of interest.

A. Integrations that Disrupt an Endogenous Locus

In another embodiment, a method of the invention may be used to integrate a mutation that disrupts an endogenous locus. For instance, a chromosomal sequence may be disrupted by the substitution of an exogenous sequence for an endogenous sequence, such that the exogenous sequence is under the control of the endogenous promoter. In these embodiments, the disrupted endogenous sequence would not be expressed, but the integrated exogenous sequence would be expressed. The exogenous sequence may be a homolog of the endogenous sequence. For instance, the exogenous sequence may be a human sequence when the endogenous sequence is non-human. In some embodiments, the exogenous sequence may be unrelated to the endogenous sequence it is replacing. For instance, an endogenous sequence may be substituted for an exogenous marker such that when the endogenous promoter is active, the marker is detectable. In some embodiments, the marker may be an enzymatic marker that can amplify the detectable signal of the marker.

Alternatively, in some embodiments a method of the invention may be used to substitute an endogenous promoter or other regulatory sequence with an exogenous promoter or regulator sequence. In these embodiments, the expression pattern of the locus would be dictated by the exogenous promoter or regulatory sequence, as opposed to the endogenous promoter or regulatory sequence. Such an exogenous promoter or regulatory sequence may be a homolog of the endogenous promoter or regulatory sequence. For instance, the exogenous sequence may be a human sequence when the endogenous sequence is non-human. In some embodiments, the exogenous sequence may be unrelated to the endogenous sequence it is replacing.

### C. Integration of an Exogenous Nucleic Acid Sequence

Alternatively, instead of disrupting a locus, a method of the invention may be used to integrate an exogenous sequence, with or without a promoter, into a chromosomal sequence without disrupting the expression of an endogenous locus. In some embodiments, such integration may be in a “safe harbor” locus, such as Rosa26 locus in the rat (or an equivalent in another animal) or the HPRT locus on the X chromosome in the rat (or an equivalent in another animal).

In one embodiment, a cassette comprising an exogenous promoter operably linked to an exogenous nucleic acid sequence may be integrated into a safe harbor locus. In certain embodiments, the exogenous promoter may be conditional. For instance, a conditional promoter may be a tissue-specific promoter, an organ specific promoter, or a cell-type specific promoter (such as a stem cell promoter, a B-cell promoter, a hair cell promoter, etc.) or an inducible promoter. An inducible promoter, as used herein, is a promoter that is active only in the presence of a particular substance, such as an antibiotic, a drug, or other exogenous compound. In some embodiments, the integration of a cassette comprising a conditional promoter may be used to track cell lineages.

In another embodiment, an exogenous nucleic acid sequence may be integrated to serve as a detectable marker for a particular nucleic acid sequence.

### D. Humanized

In an additional embodiment, the genetically modified animal may be a “humanized” animal comprising at least one chromosomally integrated sequence encoding a functional human protein. The functional human protein may have no corresponding ortholog in the genetically modified animal. Alternatively, the wild-type animal from which the genetically modified animal is derived may comprise an ortholog corresponding to the functional human protein. In this case, the orthologous sequence in the “humanized” animal is inactivated such that no endogenous functional protein is made and the “humanized” animal comprises at least one chromosomally integrated sequence encoding the human protein. Those of skill in the art appreciate that “humanized” animals may be generated by crossing a knock-out animal with a knock-in animal comprising the chromosomally integrated sequence.

### (g) Multiple Chromosomal Edits

A further embodiment of the above invention comprises performing a method of the invention serially, such that a cell is developed with more than one chromosomal edit. For instance, an embryo with a first edit may be cultured to produce an animal comprising the first genomic edit. An embryo deriving from this animal may then be used in a method of the invention to create a second genomic edit. The same process may be repeated to create an embryo with three, four, five, six, seven, eight, nine, ten or more than ten genomic edits.

Alternatively, a cell with multiple genomic edits may be developed by simultaneously introducing more than one zinc finger nuclease, each specific for a distinct edit site. A corresponding number of donor and/or exchange polynucleotides may optionally be introduced as well. The number of zinc finger nucleases and optional corresponding donor or exchange polynucleotides introduced into a cell may be two, three, four, five or more than five.

II. Applications Derived from a Method of the Invention

A method of the invention may be used to create an animal or cell comprising an edited chromosomal sequence. Such an animal or cell may be used for several different applications, including, for instance, research applications, livestock applications, companion animal applications, or biomolecule production applications. Non-limiting examples of such applications are detailed in sections (a)-(d) below.

### (a) Research Applications

In certain embodiments, a method of the invention may be used to create an animal or cell that may be used in research applications. Such applications may include disease models, pharmacological models, developmental models, cellular function models, and humanized models, each of which are detailed below.

i. Disease Models

A method of the invention may be used to create an animal or cell that may be used as a disease model. As used herein, “disease” refers to a disease, disorder, or indication in a subject. For instance, in one embodiment, a method of the invention may be used to create an animal or cell that comprises a chromosomal edit in one or more nucleic acid sequences associated with a disease. Such a nucleic acid sequence may encode a disease associated protein sequence or may be a disease associated control sequence.

In one embodiment, an animal or cell created by a method of the invention may be used to study the effects of mutations on the animal or cell and development and/or progression of the disease using measures commonly used in the study of the disease. Alternatively, such an animal or cell may be used to study the effect of a pharmaceutically active compound on the disease.

In another embodiment, an animal or cell created by a method of the invention may be used to assess the efficacy of a potential gene therapy strategy. That is, a chromosomal sequence encoding a protein associated with a disease may be modified such that the disease development and/or progression is inhibited or reduced. In particular, the method comprises editing a chromosomal sequence encoding a protein associated with the disease such that an altered protein is produced and, as a result, the animal or cell has an altered response. Accordingly, in some embodiments, a genetically modified animal may be compared with an animal predisposed to development of the disease such that the effect of the gene therapy event may be assessed.

In certain embodiments, a method of the invention may be used to create an animal or cell that maybe used as a disease model for a disease listed in Table A. Such an animal or cell may comprise a chromosomal edit in a gene listed in Table A. In another embodiment, a method of the invention may be used to create an animal or cell that maybe used as a disease model for a disease listed in Table B. Such an animal or cell may comprise a chromosomal edit in a gene listed in Table B. In Table B, a six-digit number following an entry in the Disease/Disorder/Indication column is an OMIM number (Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), available on the World Wide Web. A number in parentheses after the name of each disorder indicates whether the mutation was positioned by mapping the wildtype gene (1), by mapping the disease phenotype itself (2), or by both approaches (3). For example, a “(3)”, includes mapping of the wildtype gene combined with demonstration of a mutation in that gene in association with the disorder.”

TABLE A

| DISEASE/DISORDERS | GENE(S) |
|---|---|
| Neoplasia | PTEN; ATM; ATR; EGFR; ERBB2; ERBB3; ERBB4; |
| | Notch1; Notch2; Notch3; Notch4; AKT; AKT2; AKT3; HIF; |
| | HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; DAOA; |
| | DTNBP1; Dao (Dao1) |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and Atn1 |
| | (DRPLA Dx); CBP (Creb-BP - global instability); VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion - related disorders | Prp |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE B

| DISEASE/DISORDER/INDICATION | GENE(S) |
|---|---|
| 17,20-lyase deficiency, isolated, 202110 (3) | CYP17A1, CYP17, P450C17 |
| 17-alpha-hydroxylase/17,20-lyase | CYP17A1, CYP17, P450C17 |
| deficiency, 202110 (3) | |
| 2-methyl-3-hydroxybutyryl-CoA | HADH2, ERAB |
| dehydrogenase deficiency, 300438 (3) | |
| 2-methylbutyrylglycinuria (3) | ACADSB |
| 3-beta-hydroxysteroid dehydrogenase, type | HSD3B2 |
| II, deficiency (3) | |
| 3-hydroxyacyl-CoA dehydrogenase | HADHSC, SCHAD |
| deficiency, 609609 (3) | |
| 3-Methylcrotonyl-CoA carboxylase 1 | MCCC1, MCCA |
| deficiency, 210200 (3) | |
| 3-Methylcrotonyl-CoA carboxylase 2 | MCCC2, MCCB |
| deficiency, 210210 (3) | |
| 3-methylglutaconic aciduria, type I, 250950 | AUH |
| (3) | |
| 3-methylglutaconicaciduria, type III, 258501 | OPA3, MGA3 |
| (3) | |
| 3-M syndrome, 273750 (3) | CUL7 |
| 6-mercaptopurine sensitivity (3) | TPMT |
| Aarskog-Scott syndrome (3) | FGD1, FGDY, AAS |
| Abacavir hypersensitivity, susceptibility to | HLA-B |
| (3) | |
| ABCD syndrome, 600501 (3) | EDNRB, HSCR2, ABCDS |
| Abetalipoproteinemia, 200100 (3) | MTP |
| Abetalipoproteinemia (3) | APOB, FLDB |
| Acampomelic campolelic dysplasia, 114290 | SOX9, CMD1, SRA1 |
| (3) | |
| Acatalasemia (3) | CAT |
| Accelerated tumor formation, susceptibility | MDM2 |
| to (3) | |
| Achalasia-addisonianism-alacrimia | AAAS, AAA |
| syndrome, 231550 (3) | |
| Acheiropody, 200500 (3) | C7orf2, ACHP, LMBR1 |
| Achondrogenesis-hypochondrogenesis, | COL2A1 |
| type II, 200610 (3) | |
| Achondrogenesis Ib, 600972 (3) | SLC26A2, DTD, DTDST, D5S1708, |
| | EDM4 |
| Achondroplasia, 100800 (3) | FGFR3, ACH |
| Achromatopsia-2, 216900 (3) | CNGA3, CNG3, ACHM2 |
| Achromatopsia-3, 262300 (3) | CNGB3, ACHM3 |
| Achromatopsia-4 (3) | GNAT2, ACHM4 |
| Acid-labile subunit, deficiency of (3) | IGFALS, ALS |
| Acquired long QT syndrome, susceptibility | KCNH2, LQT2, HERG |
| to (3) | |
| Acrocallosal syndrome, 200990 (3) | GLI3, PAPA, PAPB, ACLS |
| Acrocapitofemoral dysplasia, 607778 (3) | IHH, BDA1 |
| Acrodermatitis enteropathica, 201100 (3) | SLC39A4, ZIP4 |
| Acrokeratosis verruciformis, 101900 (3) | ATP2A2, ATP2B, DAR |
| Acromegaly, 102200 (3) | GNAS, GNAS1, GPSA, POH, PHP1B, |
| | PHP1A, AHO |
| Acromegaly, 102200 (3) | SSTR5 |
| Acromesomelic dysplasia, Hunter- | GDF5, CDMP1 |
| Thompson type, 201250 (3) | |
| Acromesomelic dysplasia, Maroteaux type, | NPR2, ANPRB, AMDM |
| 602875 (3) | |
| Acyl-CoA dehydrogenase, long chain, | ACADL, LCAD |
| deficiency of (3) | |
| Acyl-CoA dehydrogenase, medium chain, | ACADM, MCAD |
| deficiency of, 201450 (3) | |
| Acyl-CoA dehydrogenase, short-chain, | ACADS, SCAD |
| deficiency of, 201470 (3) | |
| Adenocarcinoma of lung, response to | EGFR |
| tyrosine kinase inhibitor in, 211980 (3) | |
| Adenocarcinoma of lung, somatic, 211980 | BRAF |
| (3) | |
| Adenocarcinoma of lung, somatic, 211980 | ERBB2, NGL, NEU, HER2 |
| (3) | |
| Adenocarcinoma of lung, somatic, 211980 | PRKN, PARK2, PDJ |
| (3) | |
| Adenocarcinoma, ovarian, somatic (3) | PRKN, PARK2, PDJ |
| Adenoma, periampullary (3) | APC, GS, FPC |
| Adenomas, multiple colorectal, 608456 (3) | MUTYH |
| Adenomas, salivary gland pleomorphic, | PLAG1, SGPA, PSA |
| 181030 (3) | |
| Adenomatous polyposis coli (3) | APC, GS, FPC |
| Adenomatous polyposis coli, attenuated (3) | APC, GS, FPC |
| Adenosine deaminase deficiency, partial, | ADA |
| 102700 (3) | |
| Adenylosuccinase deficiency, 103050 (3) | ADSL |
| Adiponectin deficiency (3) | APM1, GBP28 |
| Adrenal adenoma, sporadic (3) | MEN1 |
| Adrenal cortical carcinoma, 202300 (3) | TP53, P53, LFS1 |
| Adrenal hyperplasia, congenital, due to 11- | CYP11B1, P450C11, FHI |
| beta-hydroxylase deficiency (3) | |
| Adrenal hyperplasia, congenital, due to 21- | CYP21A2, CYP21, CA21H |
| hydroxylase deficiency (3) | |
| Adrenal hyperplasia, congenital, due to | POR |
| combined P450C17 and P450C21 | |
| deficiency, 201750 (3) | |
| Adrenal hypoplasia, congenital, with | DAX1, AHC, AHX, NROB1 |
| hypogonadotropic hypogonadism, 300200 | |
| (3) | |
| Adrenocortical insufficiency without ovarian | FTZF1, FTZ1, SF1 |
| defect (3) | |
| Adrenocortical tumor, somatic (3) | PRKAR1A, TSE1, CNC1, CAR |
| Adrenocorticotropic hormone deficiency, | TBS19 |
| 201400 (3) | |
| Adrenoleukodystrophy, 300100 (3) | ABCD1, ALD, AMN |
| Adrenoleukodystrophy, neonatal, 202370 | PEX10, NALD |
| (3) | |
| Adrenoleukodystrophy, neonatal, 202370 | PEX13, ZWS, NALD |
| (3) | |
| Adrenoleukodystrophy, neonatal, 202370 | PEX1, ZWS1 |
| (3) | |
| Adrenoleukodystrophy, neonatal, 202370 | PEX26 |
| (3) | |
| Adrenoleukodystrophy, neonatal, 202370 | PXR1, PEX5, PTS1R |
| (3) | |
| Adrenomyeloneuropathy, 300100 (3) | ABCD1, ALD, AMN |
| Adult i phenotype with congenital cataract, | GCNT2 |
| 110800 (3) | |
| Adult i phenotype without cataract, 110800 | GCNT2 |
| (3) | |
| ADULT syndrome, 103285 (3) | TP73L, TP63, KET, EEC3, SHFM4, |
| | LMS, RHS |
| Advanced sleep phase syndrome, familial, | PER2, FASPS, KIAA0347 |
| 604348 (3) | |
| Afibrinogenemia, 202400 (3) | FGA |
| Afibrinogenemia, congenital, 202400 (3) | FGB |
| Agammaglobulinemia, 601495 (3) | IGHM, MU |
| Agammaglobulinemia, autosomal recessive | IGLL1, IGO, IGL5, VPREB2 |
| (3) | |
| Agammaglobulinemia, non-Bruton type, | LRRC8, KIAA1437 |
| 601495 (3) | |
| Agammaglobulinemia, type 1, X-linked (3) | BTK, AGMX1, IMD1, XLA, AT |
| AGAT deficiency (3) | GATM, AGAT |
| Agenesis of the corpus callosum with | SLC12A6, KCC3A, KCC3B, KCC3, |
| peripheral neuropathy, 218000 (3) | ACCPN |
| AICA-ribosiduria due to ATIC deficiency, | ATIC, PURH, AICAR |
| 608688 (3) | |
| AIDS, delayed/rapid progression to (3) | KIR3DL1, NKAT3, NKB1, AMB11, |
| | KIR3DS1 |
| AIDS, rapid progression to, 609423 (3) | IFNG |
| AIDS, resistance to (3) | CXCL12, SDF1 |
| Alagille syndrome, 118450 (3) | JAG1, AGS, AHD |
| Albinism, brown oculocutaneous, (3) | OCA2, P, PED, D15S12, BOCA |
| Albinism, ocular, autosomal recessive (3) | OCA2, P, PED, D15S12, BOCA |
| Albinism, oculocutaneous, type IA, 203100 | TYR |
| (3) | |
| Albinism, oculocutaneous, type IB, 606952 | TYR |
| (3) | |
| Albinism, oculocutaneous, type II (3) | OCA2, P, PED, D15S12, BOCA |
| Albinism, rufous, 278400 (3) | TYRP1, CAS2, GP75 |
| Alcohol dependence, susceptibility to, | HTR2A |
| 103780 (3) | |
| Alcohol intolerance, acute (3) | ALDH2 |
| Alcoholism, susceptibility to, 103780 (3) | GABRA2 |
| Aldolase A deficiency (3) | ALDOA |
| Aldosterone to renin ratio raised (3) | CYP11B2 |
| Aldosteronism, glucocorticoid-remediable, | CYP11B1, P450C11, FHI |
| 103900 (3) | |
| Alexander disease, 203450 (3) | GFAP |
| Alexander disease, 203450 (3) | NDUFV1, UQOR1 |
| Alkaptonuria, 203500 (3) | HGD, AKU |
| Allan-Herndon-Dudley syndrome, 300523 | SLC16A2, DXS128, XPCT |
| (3) | |
| Allergic rhinitis, susceptibility to, 607154 (3) | IL13, ALRH |
| Alopecia universalis, 203655 (3) | HR, AU |
| Alpers syndrome, 203700 (3) | POLG, POLG1, POLGA, PEO |
| Alpha-1-antichymotrypsin deficiency (3) | SERPINA3, AACT, ACT |
| Alpha-actinin-3 deficiency (3) | ACTN3 |
| Alpha-methylacetoacetic aciduria, 203750 | ACAT1 |
| (3) | |
| Alpha-methylacyl-CoA racemase deficiency | AMACR |
| (3) | |
| Alpha-thalassemia/mental retardation | ATRX, XH2, XNP, MRXS3, SHS |
| syndrome, 301040 (3) | |
| Alpha-thalassemia myelodysplasia | ATRX, XH2, XNP, MRXS3, SHS |
| syndrome, somatic, 300448 (3) | |
| Alport syndrome, 301050 (3) | COL4A5, ATS, ASLN |
| Alport syndrome, autosomal recessive, | COL4A3 |
| 203780 (3) | |
| Alport syndrome, autosomal recessive, | COL4A4 |
| 203780 (3) | |
| Alstrom syndrome, 203800 (3) | ALMS1, ALSS, KIAA0328 |
| Alternating hemiplegia of childhood, 104290 | ATP1A2, FHM2, MHP2 |
| (3) | |
| Alveolar soft-part sarcoma, 606243 (3) | ASPCR1, RCC17, ASPL, ASPS |
| Alzheimer disease-1, APP-related (3) | APP, AAA, CVAP, AD1 |
| Alzheimer disease-2, 104310 (3) | APOE, AD2 |
| Alzheimer disease-4, 606889 (3) | PSEN2, AD4, STM2 |
| Alzheimer disease, late-onset, 104300 (3) | APBB2, FE65L1 |
| Alzheimer disease, late-onset, susceptibility | NOS3 |
| to, 104300 (3) | |
| Alzheimer disease, late-onset, susceptibility | PLAU, URK |
| to, 104300 (3) | |
| Alzheimer disease, susceptibility to, 104300 | ACE, DCP1, ACE1 |
| (3) | |
| Alzheimer disease, susceptibility to, 104300 | MPO |
| (3) | |
| Alzheimer disease, susceptibility to, 104300 | PACIP1, PAXIP1L, PTIP |
| (3) | |
| Alzheimer disease, susceptibility to (3) | A2M |
| Alzheimer disease, susceptibility to (3) | BLMH, BMH |
| Alzheimer disease, type 3, 607822 (3) | PSEN1, AD3 |
| Alzheimer disease, type 3, with spastic | PSEN1, AD3 |
| paraparesis and apraxia, 607822 (3) | |
| Alzheimer disease, type 3, with spastic | PSEN1, AD3 |
| paraparesis and unusual plaques, 607822 | |
| (3) | |
| Amelogenesis imperfecta 2, hypoplastic | ENAM |
| local, 104500 (3) | |
| Amelogenesis imperfecta, 301200 (3) | AMELX, AMG, AIH1, AMGX |
| Amelogenesis imperfecta, hypomaturation- | DLX3, TDO |
| hypoplastic type, with taurodontism, 104510 | |
| (3) | |
| Amelogenesis imperfecta, hypoplastic, and | ENAM |
| openbite malocclusion, 608563 (3) | |
| Amelogenesis imperfecta, pigmented | KLK4, EMSP1, PRSS17 |
| hypomaturation type, 204700 (3) | |
| Amish infantile epilepsy syndrome, 609056 | SIAT9, ST3GALV |
| (3) | |
| AMP deaminase deficiency, erythrocytic (3) | AMPD3 |
| Amyloid neuropathy, familial, several allelic | TTR, PALB |
| types (3) | |
| Amyloidosis, 3 or more types (3) | APOA1 |
| Amyloidosis, cerebroarterial, Dutch type (3) | APP, AAA, CVAP, AD1 |
| Amyloidosis, Finnish type, 105120 (3) | GSN |
| Amyloidosis, hereditary renal, 105200 (3) | FGA |
| Amyloidosis, renal, 105200 (3) | LYZ |
| Amyloidosis, senile systemic (3) | TTR, PALB |
| Amyotrophic lateral sclerosis 8, 608627 (3) | VAPB, VAPC, ALS8 |
| Amyotrophic lateral sclerosis, due to SOD1 | SOD1, ALS1 |
| deficiency, 105400 (3) | |
| Amyotrophic lateral sclerosis, juvenile, | ALS2, ALSJ, PLSJ, IAHSP |
| 205100 (3) | |
| Amyotrophic lateral sclerosis, susceptibility | DCTN1 |
| to, 105400 (3) | |
| Amyotrophic lateral sclerosis, susceptibility | NEFH |
| to, 105400 (3) | |
| Amyotrophic lateral sclerosis, susceptibility | PRPH |
| to, 105400 (3) | |
| Analbuminemia (3) | ALB |
| Analgesia from kappa-opioid receptor | MC1R |
| agonist, female-specific (3) | |
| Anderson disease, 607689 (3) | SARA2, SAR1B, CMRD |
| Androgen insensitivity, 300068 (3) | AR, DHTR, TFM, SBMA, KD, SMAX1 |
| Anemia, congenital dyserythropoietic, type I, | CDAN1, CDA1 |
| 224120 (3) | |
| Anemia, Diamond-Blackfan, 105650 (3) | RPS19, DBA |
| Anemia, hemolytic, due to PK deficiency (3) | PKLR, PK1 |
| Anemia, hemolytic, due to UMPH1 | NT5C3, UMPH1, PSN1 |
| deficiency, 266120 (3) | |
| Anemia, hemolytic, Rh-null, regulator type, | RHAG, RH50A |
| 268150 (3) | |
| Anemia, hypochromic microcytic, 206100 | NRAMP2 |
| (3) | |
| Anemia, neonatal hemolytic, fatal and near- | SPTB |
| fatal (3) | |
| Anemia, sideroblastic/hypochromic (3) | ALAS2, ANH1, ASB |
| Anemia, sideroblastic, with ataxia, 301310 | ABCB7, ABC7, ASAT |
| (3) | |
| Aneurysm, familial arterial (3) | COL3A1 |
| Angelman syndrome, 105830 (3) | MECP2, RTT, PPMX, MRX16, MRX79 |
| Angelman syndrome, 105830 (3) | UBE3A, ANCR |
| Angioedema, hereditary, 106100 (3) | C1NH, HAE1, HAE2, SERPING1 |
| Angioedema induced by ACE inhibitors, | XPNPEP2 |
| susceptibility to (3) | |
| Angiofibroma, sporadic (3) | MEN1 |
| Angiotensin I-converting enzyme, benign | ACE, DCP1, ACE1 |
| serum increase (3) | |
| Anhaptoglobinemia (3) | HP |
| Aniridia, type II, 106210 (3) | PAX6, AN2, MGDA |
| Ankylosing spoldylitis, susceptibility to, | HLA-B |
| 106300 (3) | |
| Anophthalmia 3, 206900 (3) | SOX2, ANOP3 |
| Anorexia nervosa, susceptibility to, 606788 | HTR2A |
| (3) | |
| Anterior segment anomalies and cataract | EYA1, BOR |
| (3) | |
| Anterior segment mesenchymal dysgenesis, | FOXE3, FKHL12, ASMD |
| 107250 (3) | |
| Anterior segment mesenchymal dysgenesis | FOXC1, FKHL7, FREAC3 |
| (3) | |
| Anterior segment mesenchymal dysgenesis | PITX3 |
| and cataract, 107250 (3) | |
| Antithrombin III deficiency (3) | AT3 |
| Antley-Bixler syndrome, 207410 (3) | POR |
| Anxiety-related personality traits (3) | SLC6A4, HTT, OCD1 |
| Aortic aneurysm, ascending, and dissection | FBN1, MFS1, WMS |
| (3) | |
| Apert syndrome, 101200 (3) | FGFR2, BEK, CFD1, JWS |
| Aplasia of lacrimal and salivary glands, | FGF10 |
| 180920 (3) | |
| Aplastic anemia, 609135 (3) | IFNG |
| Aplastic anemia, 609135 (3) | TERC, TRC3, TR |
| Aplastic anemia, susceptibility to, 609135 | TERT, TCS1, EST2 |
| (3) | |
| Apnea, postanesthetic (3) | BCHE, CHE1 |
| ApoA-I and apoC-III deficiency, combined | APOA1 |
| (3) | |
| Apolipoprotein A-II deficiency (3) | APOA2 |
| Apolipoprotein C3 deficiency (3) | APOC3 |
| Apolipoprotein H deficiency (3) | APOH |
| Apparent mineralocorticoid excess, | HSD11B2, HSD11K |
| hypertension due to (3) | |
| Aquaporin-1 deficiency (3) | AQP1, CHIP28, CO |
| ARC syndrome, 208085 (3) | VPS33B |
| Argininemia, 207800 (3) | ARG1 |
| Argininosuccinic aciduria, 207900 (3) | ASL |
| Aromatase deficiency (3) | CYP19A1, CYP19, ARO |
| Aromatic L-amino acid decarboxylase | DDC |
| deficiency, 608643 (3) | |
| Arrhythmogenic right ventricular dysplasia 2, | RYR2, VTSIP |
| 600996 (3) | |
| Arrhythmogenic right ventricular dysplasia 8, | DSP, KPPS2, PPKS2 |
| 607450 (3) | |
| Arrhythmogenic right ventricular dysplasia, | PKP2, ARVD9 |
| familial, 9, 609040 (3) | |
| Arthrogryposis multiplex congenita, distal, | TPM2, TMSB, AMCD1, DA1 |
| type 1, 108120 (3) | |
| Arthrogryposis multiplex congenita, distal, | TNNI2, AMCD2B, DA2B, FSSV |
| type 2B, 601680 (3) | |
| Arthropathy, progressive | WISP3, PPAC, PPD |
| pseudorheumatoid, of childhood, 208230 (3) | |
| Arthyrgryposis multiplex congenita, distal, | TNNT3, AMCD2B, DA2B, FSSV |
| type 2B, 601680 (3) | |
| Aspartylglucosaminuria (3) | AGA |
| Asperger syndrome, 300494 (3) | NLGN3 |
| Asperger syndrome, 300497 (3) | NLGN4, KIAA1260, AUTSX2 |
| Asthma, 600807 (3) | PHF11, NYREN34 |
| Asthma, atopic, susceptibility to (3) | MS4A2, FCER1B |
| Asthma, dimished response to | ALOX5 |
| antileukotriene treatment in, 600807 (3) | |
| Asthma, nocturnal, susceptibility to (3) | ADRB2 |
| Asthma, susceptibility to, 1, 607277 (3) | PTGDR, AS1 |
| Asthma, susceptibility to, 2, 608584 (3) | GPR154, GPRA, VRR1, PGR14 |
| Asthma, susceptibility to (3) | HNMT |
| Asthma, susceptibility to, 600807 (3) | IL12B, NKSF2 |
| Asthma, susceptibility to, 600807 (3) | IL13, ALRH |
| Asthma, susceptibility to, 600807 (3) | PLA2G7, PAFAH |
| Asthma, susceptibility to, 600807 (3) | SCGB3A2, UGRP1 |
| Asthma, susceptibility to, 600807 (3) | TNF, TNFA |
| Asthma, susceptibility to, 600807 (3) | UGB, CC10, CCSP, SCGB1A1 |
| Ataxia, cerebellar, Cayman type, 601238 (3) | ATCAY, CLAC, KIAA1872 |
| Ataxia, early-onset, with oculomotor apraxia | APTX, AOA, AOA1 |
| and hypoalbuminemia, 208920 (3) | |
| Ataxia, episodic (3) | CACNB4, EJM |
| Ataxia-ocular apraxia-2, 606002 (3) | SETX, SCAR1, AOA2 |
| Ataxia-telangiectasia, 208900 (3) | ATM, ATA, AT1 |
| Ataxia-telangiectasia-like disorder, 604391 | MRE11A, MRE11, ATLD |
| (3) | |
| Ataxia with isolated vitamin E deficiency, | TTPA, TTP1, AVED |
| 277460 (3) | |
| Atelosteogenesis II, 256050 (3) | SLC26A2, DTD, DTDST, D5S1708, |
| | EDM4 |
| Atelostogenesis, type I, 108720 (3) | FLNB, SCT, AOI |
| Athabaskan brainstem dysgenesis | HOXA1, HOX1F, BSAS |
| syndrome, 601536 (3) | |
| Atherosclerosis, susceptibility to (3) | ALOX5 |
| Atopy, 147050 (3) | SPINK5, LEKTI |
| Atopy, resistance to, 147050 (3) | HAVCR1, HAVCR |
| Atopy, susceptibility to, 147050 (3) | PLA2G7, PAFAH |
| Atopy, susceptibility to, 147050 (3) | SELP, GRMP |
| Atopy, susceptibility to (3) | IL4R, IL4RA |
| Atransferrinemia, 209300 (3) | TF |
| Atrial fibrillation, familial, 607554 (3) | KCNE2, MIRP1, LQT6 |
| Atrial fibrillation, familial, 607554 (3) | KCNQ1, KCNA9, LQT1, KVLQT1, |
| | ATFB1 |
| Atrial septal defect-2, 607941 (3) | GATA4 |
| Atrial septal defect 3 (3) | MYH6, ASD3, MYHCA |
| Atrial septal defect with atrioventricular | NKX2E, CSX |
| conduction defects, 108900 (3) | |
| Atrichia with papular lesions, 209500 (3) | HR, AU |
| Atrioventricular block, idiopathic second- | NKX2E, CSX |
| degree (3) | |
| Atrioventricular septal defect, 600309 (3) | GJA1, CX43, ODDD, SDTY3, ODOD |
| Atrioventricular septal defect, partial, with | CRELD1, AVSD2 |
| heterotaxy syndrome, 606217 (3) | |
| Atrioventricular septal defect, susceptibility | CRELD1, AVSD2 |
| to, 2, 606217 (3) | |
| Attention deficit-hyperactivity disorder, | DRD5, DRD1B, DRD1L2 |
| susceptibility to, 143465 (3) | |
| Autism, susceptibility to, 209850 (3) | GLO1 |
| Autism, X-linked, 300425 (3) | MECP2, RTT, PPMX, MRX16, MRX79 |
| Autism, X-linked, 300425 (3) | NLGN3 |
| Autism, X-linked, 300495 (3) | NLGN4, KIAA1260, AUTSX2 |
| Autoimmune lymphoproliferative syndrome, | TNFRSF6, APT1, FAS, CD95, ALPS1A |
| 601859 (3) | |
| Autoimmune lymphoproliferative syndrome, | TNFRSF6, APT1, FAS, CD95, ALPS1A |
| type IA, 601859 (3) | |
| Autoimmune lymphoproliferative syndrome, | CASP10, MCH4, ALPS2 |
| type II, 603909 (3) | |
| Autoimmune lymphoproliferative syndrome, | CASP8, MCH5 |
| type IIB, 607271 (3) | |
| Autoimmune polyglandular disease, type I, | AIRE, APECED |
| 240300 (3) | |
| Autoimmune thyroid disease, susceptibility | TG, AITD3 |
| to 3, 608175 (3) | |
| Autonomic nervous system dysfunction (3) | DRD4 |
| Axenfeld anomaly (3) | FOXC1, FKHL7, FREAC3 |
| Azoospermia (3) | USP9Y, DFFRY |
| Azoospermia due to perturbations of | SYCP3, SCP3, COR1 |
| meiosis, 270960 (3) | |
| Bamforth-Lazarus syndrome, 241850 (3) | FOXE1, FKHL15, TITF2, TTF2 |
| Bannayan-Riley-Ruvalcaba syndrome, | PTEN, MMAC1 |
| 153480 (3) | |
| Bannayan-Zonana syndrome, 153480 (3) | PTEN, MMAC1 |
| Bardet-Biedl syndrome 1, 209900 (3) | BBS1 |
| Bardet-Biedl syndrome 1, modifier of, | ARL6, BBS3 |
| 209900 (3) | |
| Bardet-Biedl syndrome, 209900 (3) | BBS7 |
| Bardet-Biedl syndrome 2, 209900 (3) | BBS2 |
| Bardet-Biedl syndrome 3, 600151 (3) | ARL6, BBS3 |
| Bardet-Biedl syndrome 4, 209900 (3) | BBS4 |
| Bardet-Biedl syndrome 5, 209900 (3) | BBS5 |
| Bardet-Biedl syndrome 6, 209900 (3) | MKKS, HMCS, KMS, MKS, BBS6 |
| Bardet-Biedl syndrome 8, 209900 (3) | TTC8, BBS8 |
| Bare lymphocyte syndrome, type I, 604571 | TAPBP, TPSN |
| (3) | |
| Bare lymphocyte syndrome, type I, due to | TAP2, ABCB3, PSF2, RING11 |
| TAP2 deficiency, 604571 (3) | |
| Bare lymphocyte syndrome, type II, | MHC2TA, C2TA |
| complementation group A, 209920 (3) | |
| Bare lymphocyte syndrome, type II, | RFX5 |
| complementation group C, 209920 (3) | |
| Bare lymphocyte syndrome, type II, | RFXAP |
| complementation group D, 209920 (3) | |
| Bare lymphocyte syndrome, type II, | RFX5 |
| complementation group E, 209920 (3) | |
| Barth syndrome, 302060 (3) | TAZ, EFE2, BTHS, CMD3A, LVNCX |
| Bart-Pumphrey syndrome, 149200 (3) | GJB2, CX26, DFNB1, PPK, DFNA3, |
| | KID, HID |
| Bartter syndrome, type 1, 601678 (3) | SLC12A1, NKCC2 |
| Bartter syndrome, type 2, 241200 (3) | KCNJ1, ROMK1 |
| Bartter syndrome, type 3, 607364 (3) | CLCNKB |
| Bartter syndrome, type 4, 602522 (3) | BSND |
| Bartter syndrome, type 4, digenic, 602522 | CLCNKA |
| (3) | |
| Bartter syndrome, type 4, digenic, 602522 | CLCNKB |
| (3) | |
| Basal cell carcinoma (3) | RASA1, GAP, CMAVM, PKWS |
| Basal cell carcinoma, somatic, 605462 (3) | PTCH2 |
| Basal cell carcinoma, somatic, 605462 (3) | PTCH, NBCCS, BCNS, HPE7 |
| Basal cell carcinoma, sporadic (3) | SMOH, SMO |
| Basal cell nevus syndrome, 109400 (3) | PTCH, NBCCS, BCNS, HPE7 |
| Basal ganglia disease, adult-onset, 606159 | FTL |
| (3) | |
| Basal ganglia disease, biotin-responsive, | SLC19A3 |
| 607483 (3) | |
| B-cell non-Hodgkin lymphoma, high-grade | BCL7A, BCL7 |
| (3) | |
| BCG infection, generalized familial (3) | IFNGR1 |
| Beare-Stevenson cutis gyrata syndrome, | FGFR2, BEK, CFD1, JWS |
| 123790 (3) | |
| Becker muscular dystrophy, 300376 (3) | DMD, BMD |
| Becker muscular dystrophy modifier, | MYF6 |
| 310200 (3) | |
| Beckwith-Wiedemann syndrome, 130650 | CDKN1C, KIP2, BWS |
| (3) | |
| Beckwith-Wiedemann syndrome, 130650 | H19, D11S813E, ASM1, BWS |
| (3) | |
| Beckwith-Wiedemann syndrome, 130650 | KCNQ10T1, LIT1 |
| (3) | |
| Beckwith-Wiedemann syndrome, 130650 | NSD1, ARA267, STO |
| (3) | |
| Benzene toxicity, susceptibility to (3) | NQO1, DIA4, NMOR1 |
| Bernard-Soulier syndrome, 231200 (3) | GP1BA |
| Bernard-Soulier syndrome, type B, 231200 | GP1BB |
| (3) | |
| Bernard-Soulier syndrome, type C (3) | GP9 |
| Beryllium disease, chronic, susceptibility to | HLA-DPB1 |
| (3) | |
| Beta-2-adrenoreceptor agonist, reduced | ADRB2 |
| response to (3) | |
| Beta-ureidopropionase deficiency (3) | UPB1, BUP1 |
| Bethlem myopathy, 158810 (3) | COL6A1, OPLL |
| Bethlem myopathy, 158810 (3) | COL6A2 |
| Bethlem myopathy, 158810 (3) | COL6A3 |
| Bietti crystalline corneoretinal dystrophy, | CYP4V2, BCD |
| 210370 (3) | |
| Bile acid malabsorption, primary (3) | SLC10A2, NTCP2 |
| Biotinidase deficiency, 253260 (3) | BTD |
| Bipolar disorder, susceptibility to, 125480 | XBP1, XBP2 |
| (3) | |
| Birt-Hogg-Dube syndrome, 135150 (3) | FLCN, BHD |
| Bladder cancer, 109800 (3) | FGFR3, ACH |
| Bladder cancer, 109800 (3) | KRAS2, RASK2 |
| Bladder cancer, 109800 (3) | RB1 |
| Bladder cancer, somatic, 109800 (3) | HRAS |
| Blau syndrome, 186580 (3) | CARD15, NOD2, IBD1, CD, ACUG, |
| | PSORAS1 |
| Bleeding disorder due to defective | TBXA2R |
| thromboxane A2 receptor (3) | |
| Bleeding due to platelet ADP receptor | P2RX1, P2X1 |
| defect, 600515 (3) | |
| Blepharophimosis, epicanthus inversus, and | FOXL2, BPES, BPES1, PFRK, POF3 |
| ptosis, type 1, 110100 (3) | |
| Blepharophimosis, epicanthus inversus, and | FOXL2, BPES, BPES1, PFRK, POF3 |
| ptosis, type 2, 110100 (3) | |
| Blepharospasm, primary benign, 606798 (3) | DRD5, DRD1B, DRD1L2 |
| Blood group, ABO system (3) | ABO |
| Blood group, Auberger system (3) | LU, AU, BCAM |
| Blood group, Colton, 110450 (3) | AQP1, CHIP28, CO |
| Blood group Cromer (3) | DAF |
| Blood group, Diego, 110500 (3) | SLC4A1, AE1, EPB3 |
| Blood group, Dombrock (3) | ART4, DO |
| Blood group, Gerbich (3) | GYPC, GE, GPC |
| Blood group GIL, 607457 (3) | AQP3 |
| Blood group, li, 110800 (3) | GCNT2 |
| Blood group, Indian system (3) | CD44, MDU2, MDU3, MIC4 |
| Blood group, Kell (3) | KEL |
| Blood group, Kidd (3) | SLC14A1, JK, UTE, UT1 |
| Blood group, Knops system, 607486 (3) | CR1, C3BR |
| Blood group, Landsteiner-Wiener (3) | LW |
| Blood group, Lewis (3) | FUT3, LE |
| Blood group, Lutheran system (3) | LU, AU, BCAM |
| Blood group, MN (3) | GYPA, MN, GPA |
| Blood group, OK, 111380 (3) | BSG |
| Blood group, P system, 111400 (3) | A4GALT, PK |
| Blood group, P system, 111400 (3) | B3GALT3, GLCT3, P |
| Blood group, Rhesus (3) | RHCE |
| Blood group, Ss (3) | GYPB, SS, MNS |
| Blood group, Waldner, 112010 (3) | SLC4A1, AE1, EPB3 |
| Blood group, Wright, 112050 (3) | SLC4A1, AE1, EPB3 |
| Blood group, XG system (3) | XG |
| Blood group, Yt system, 112100 (3) | ACHE, YT |
| Bloom syndrome, 210900 (3) | RECQL3, RECQ2, BLM, BS |
| Blue-cone monochromacy, 303700 (3) | OPN1LW, RCP, CBP, CBBM |
| Blue-cone monochromacy, 303700 (3) | OPN1MW, GCP, CBD, CBBM |
| Bombay phenotype (3) | FUT1, H, HH |
| Bombay phenotype (3) | FUT2, SE |
| Bone mineral density variability 1, 601884 | LRP5, BMND1, LRP7, LR3, OPPG, |
| (3) | VBCH2 |
| Borjeson-Forssman-Lehmann syndrome, | PHF6, BFLS |
| 301900 (3) | |
| Bosley-Salih-Alorainy syndrome, 601536 (3) | HOXA1, HOX1F, BSAS |
| Bothnia retinal dystrophy, 607475 (3) | RLBP1 |
| Brachydactyly, type A1, 112500 (3) | IHH, BDA1 |
| Brachydactyly, type A2, 112600 (3) | BMPR1B, ALK6 |
| Brachydactyly, type B1, 113000 (3) | ROR2, BDB1, BDB, NTRKR2 |
| Brachydactyly, type C, 113100 (3) | GDF5, CDMP1 |
| Brachydactyly, type D, 113200 (3) | HOXD13, HOX4I, SPD |
| Brachydactyly, type E, 113300 (3) | HOXD13, HOX4I, SPD |
| Bradyopsia, 608415 (3) | R9AP, RGS9, PERRS |
| Bradyopsia, 608415 (3) | RGS9, PERRS |
| Branchiootic syndrome (3) | EYA1, BOR |
| Branchiootorenal syndrome, 113650 (3) | EYA1, BOR |
| Branchiootorenal syndrome with cataract, | EYA1, BOR |
| 113650 (3) | |
| Breast and colorectal cancer, susceptibility | CHEK2, RAD53, CHK2, CDS1, LFS2 |
| to (3) | |
| Breast cancer, 114480 (3) | PIK3CA |
| Breast cancer, 114480 (3) | PPM1D, WIP1 |
| Breast cancer, 114480 (3) | SLC22A1L, BWSCR1A, IMPT1 |
| Breast cancer, 114480 (3) | TP53, P53, LFS1 |
| Breast cancer-1 (3) | BRCA1, PSCP |
| Breast cancer 2, early onset (3) | BRCA2, FANCD1 |
| Breast cancer (3) | TSG101 |
| Breast cancer, early-onset, 114480 (3) | BRIP1, BACH1, FANCJ |
| Breast cancer, invasive intraductal (3) | RAD54L, HR54, HRAD54 |
| Breast cancer, lobular (3) | CDH1, UVO |
| Breast cancer, male, susceptibility to, | BRCA2, FANCD1 |
| 114480 (3) | |
| Breast cancer, male, with Reifenstein | AR, DHTR, TFM, SBMA, KD, SMAX1 |
| syndrome (3) | |
| Breast cancer, somatic, 114480 (3) | KRAS2, RASK2 |
| Breast cancer, somatic, 114480 (3) | RB1CC1, CC1, KIAA0203 |
| Breast cancer, sporadic (3) | PHB |
| Breast cancer, susceptibility to, 114480 (3) | ATM, ATA, AT1 |
| Breast cancer, susceptibility to, 114480 (3) | BARD1 |
| Breast cancer, susceptibility to, 114480 (3) | CHEK2, RAD53, CHK2, CDS1, LFS2 |
| Breast cancer, susceptibility to, 114480 (3) | RAD51A, RECA |
| Breast cancer, susceptibility to (3) | XRCC3 |
| Breast-ovarian cancer (3) | BRCA1, PSCP |
| Brody myopathy, 601003 (3) | ATP2A1, SERCA1 |
| Bruck syndrome 2, 609220 (3) | PLOD2 |
| Brugada syndrome, 601144 (3) | SCN5A, LQT3, IVF, HB1, SSS1 |
| Brunner syndrome (3) | MAOA |
| Burkitt lymphoma, 113970 (3) | MYC |
| Buschke-Ollendorff syndrome, 166700 (3) | LEMD3, MAN1 |
| Butterfly dystrophy, retinal, 169150 (3) | RDS, RP7, PRPH2, PRPH, AVMD, |
| | AOFMD |
| C1q deficiency, type A (3) | C1QA |
| C1q deficiency, type B (3) | C1QB |
| C1q deficiency, type C (3) | C1QG |
| C1s deficiency, isolated (3) | C1S |
| C2 deficiency (3) | C2 |
| C3b inactivator deficiency (3) | IF |
| C3 deficiency (3) | C3 |
| C4 deficiency (3) | C4A, C4S |
| C4 deficiency (3) | C4B, C4F |
| C6 deficiency (3) | C6 |
| C7 deficiency (3) | C7 |
| C8 deficiency, type II (3) | C8B |
| C9 deficiency (3) | C9 |
| C9 deficiency with dermatomyositis (3) | C9 |
| Cafe-au-lait spots, multiple, with leukemia, | MSH2, COCA1, FCC1, HNPCC1 |
| 114030 (3) | |
| Cafe-au-lait spots with glioma or leukemia, | MLH1, COCA2, HNPCC2 |
| 114030 (3) | |
| Caffey disease, 114000 (3) | COL1A1 |
| Calcinosis, tumoral, 211900 (3) | FGF23, ADHR, HPDR2, PHPTC |
| Calcinosis, tumoral, 211900 (3) | GALNT3 |
| Campomelic dysplasia, 114290 (3) | SOX9, CMD1, SRA1 |
| Campomelic dysplasia with autosomal sex | SOX9, CMD1, SRA1 |
| reversal, 114290 (3) | |
| Camptodactyly-arthropathy-coxa vara- | PRG4, CACP, MSF, SZP, HAPO |
| pericarditis syndrome, 208250 (3) | |
| Camurati-Engelmann disease, 131300 (3) | TGFB1, DPD1, CED |
| Canavan disease, 271900 (3) | ASPA |
| Cancer progression/metastasis (3) | FGFR4 |
| Cancer susceptibility (3) | MSH6, GTBP, HNPCC5 |
| Capillary malformation-arteriovenous | RASA1, GAP, CMAVM, PKWS |
| malformation, 608354 (3) | |
| Carbamoylphosphate synthetase I | CPS1 |
| deficiency, 237300 (3) | |
| Carbohydrate-deficient glycoprotein | PMM2, CDG1 |
| syndrome, type I, 212065 (3) | |
| Carbohydrate-deficient glycoprotein | MPI, PMI1 |
| syndrome, type Ib, 602579 (3) | |
| Carbohydrate-deficient glycoprotein | MGAT2, CDGS2 |
| syndrome, type II, 212066 (3) | |
| Carboxypeptidase N deficiency, 212070 (3) | CPN1, SCPN, CPN |
| Carcinoid tumor of lung (3) | MEN1 |
| Carcinoid tumors, intestinal, 114900 (3) | SDHD, PGL1 |
| Cardioencephalomyopathy, fatal infantile, | SCO2 |
| due to cytochrome c oxidase deficiency, | |
| 604377 (3) | |
| Cardiomyopathy, Familial hypertrophic, 8, | MYL3, CMH8 |
| 608751 (3) | |
| Cardiomyopathy, dilated, 115200 (3) | ACTC |
| Cardiomyopathy, dilated, 115200 (3) | MYH7, CMH1, MPD1 |
| Cardiomyopathy, dilated, 1A, 115200 (3) | LMNA, LMN1, EMD2, FPLD, CMD1A, |
| | HGPS, LGMD1B |
| Cardiomyopathy, dilated, 1D, 601494 (3) | TNNT2, CMH2, CMD1D |
| Cardiomyopathy, dilated, 1G, 604145 (3), | TTN, CMD1G, TMD, LGMD2J |
| Tibial muscular dystrophy, tardive, 600334 | |
| (3) | |
| Cardiomyopathy, dilated, 1I, 604765 (3) | DES, CMD1I |
| Cardiomyopathy, dilated, 1J, 605362 (3) | EYA4, DFNA10, CMD1J |
| Cardiomyopathy, dilated, 1L, 606685 (3) | SGCD, SGD, LGMD2F, CMD1L |
| Cardiomyopathy, dilated, 1M, 607482 (3) | CSRP3, CRP3, CLP, CMD1M |
| Cardiomyopathy, dilated, 1N, 607487 (3) | TCAP, LGMD2G, CMD1N |
| Cardiomyopathy, dilated, with ventricular | ABCC9, SUR2 |
| tachycardia, 608569 (3) | |
| Cardiomyopathy, dilated, X-linked, 302045 | DMD, BMD |
| (3) | |
| Cardiomyopathy, familial hypertrophic, 10, | MYL2, CMH10 |
| 608758 (3) | |
| Cardiomyopathy, familial hypertrophic, 1, | MYH7, CMH1, MPD1 |
| 192600 (3) | |
| Cardiomyopathy, familial hypertrophic, | ACTC |
| 192600 (3) | |
| Cardiomyopathy, familial hypertrophic, | CAV3, LGMD1C |
| 192600 (3) | |
| Cardiomyopathy, familial hypertrophic, | MYH6, ASD3, MYHCA |
| 192600 (3) | |
| Cardiomyopathy, familial hypertrophic, | TNNC1 |
| 192600 (3) ( ) | |
| Cardiomyopathy, familial hypertrophic, 2, | TNNT2, CMH2, CMD1D |
| 115195 (3) | |
| Cardiomyopathy, familial hypertrophic, 3, | TPM1, CMH3 |
| 115196 (3) | |
| Cardiomyopathy, familial hypertrophic (3) | TNNI3 |
| Cardiomyopathy, familial hypertrophic, 4, | MYBPC3, CMH4 |
| 115197 (3) | |
| Cardiomyopathy, familial hypertrophic, 9 (3) | TTN, CMD1G, TMD, LGMD2J |
| Cardiomyopathy, familial restrictive, 115210 | TNNI3 |
| (3) | |
| Cardiomyopathy, hypertrophic, early-onset | COX15 |
| fatal (3) | |
| Cardiomyopathy, hypertrophic, mid-left | MYL2, CMH10 |
| ventricular chamber type, 608758 (3) | |
| Cardiomyopathy, hypertrophic, | MYLK2, MLCK |
| midventricular, digenic, 192600 (3) | |
| Cardiomyopathy, hypertrophic, with WPW, | PRKAG2, WPWS |
| 600858 (3) | |
| Cardiomyopathy, idiopathic dilated, 115200 | PLN, PLB |
| (3) | |
| Cardiomyopathy, X-linked dilated, 300069 | TAZ, EFE2, BTHS, CMD3A, LVNCX |
| (3) | |
| Carney complex, type 1, 160980 (3) | PRKAR1A, TSE1, CNC1, CAR |
| Carney complex variant, 608837 (3) | MYH8 |
| Carnitine-acylcarnitine translocase | SLC25A20, CACT, CAC |
| deficiency (3) | |
| Carnitine deficiency, systemic primary, | SLC22A5, OCTN2, CDSP, SCD |
| 212140 (3) | |
| Carpal tunnel syndrome, familial (3) | TTR, PALB |
| Cartilage-hair hypoplasia, 250250 (3) | RMRP, RMRPR, CHH |
| Cataract, autosomal dominant nuclear (3) | CRYAA, CRYA1 |
| Cataract, cerulean, type 2, 601547 (3) | CRYBB2, CRYB2 |
| Cataract, congenital (3) | PITX3 |
| Cataract, congenital, 604219 (3) | BFSP2, CP49, CP47 |
| Cataract, congenital progressive, autosomal | CRYAA, CRYA1 |
| recessive (3) | |
| Cataract, congenital, with late-onset corneal | PAX6, AN2, MGDA |
| dystrophy (3) | |
| Cataract, congenital zonular, with sutural | CRYBA1, CRYB1 |
| opacities, 600881 (3) | |
| Cataract, Coppock-like, 604307 (3) | CRYGC, CRYG3, CCL |
| Cataract, cortical pulverulent, late-onset (3) | LIM2, MP19 |
| Cataract, crystalline aculeiform, 115700 (3) | CRYGD, CRYG4 |
| Cataract, juvenile-onset, 604219 (3) | BFSP2, CP49, CP47 |
| Cataract, lamellar, 116800 (3) | HSF4, CTM |
| Cataract, Marner type, 116800 (3) | HSF4, CTM |
| Cataract, polymorphic and lamellar, 604219 | MIP, AQP0 |
| (3) | |
| Cataract, posterior polar 2 (3) | CRYAB, CRYA2, CTPP2 |
| Cataract, pulverulent (3) | CRYBB1 |
| Cataracts, punctate, progressive juvenile- | CRYGD, CRYG4 |
| onset (3) | |
| Cataract, sutural, with punctate and | CRYBB2, CRYB2 |
| cerulean opacities, 607133 (3) | |
| Cataract, variable zonular pulverulent (3) | CRYGC, CRYG3, CCL |
| Cataract, zonular central nuclear, autosomal | CRYAA, CRYA1 |
| dominant (3) | |
| Cataract, zonular pulverulent-1, 116200 (3) | GJA8, CX50, CAE1 |
| Cataract, zonular pulverulent-3, 601885 (3) | GJA3, CX46, CZP3, CAE3 |
| Cavernous malformations of CNS and | CCM1, CAM, KRIT1 |
| retina, 116860 (3) | |
| CD59 deficiency (3) | CD59, MIC11 |
| CD8 deficiency, familial, 608957 (3) | CD8A |
| Central core disease, 117000 (3) | RYR1, MHS, CCO |
| Central core disease, one form (3) ( ) | MYH7, CMH1, MPD1 |
| Central hypoventilation syndrome, 209880 | GDNF |
| (3) | |
| Central hypoventilation syndrome, | BDNF |
| congenital, 209880 (3) | |
| Central hypoventilation syndrome, | EDN3 |
| congenital, 209880 (3) | |
| Central hypoventilation syndrome, | PMX2B, NBPHOX, PHOX2B |
| congenital, 209880 (3) | |
| Central hypoventilation syndrome, | RET, MEN2A |
| congenital, 209880 (3) | |
| Cerebellar ataxia, 604290 (3) | CP |
| Cerebellar ataxia, pure (3) | CACNA1A, CACNL1A4, SCA6 |
| Cerebellar hypoplasia, VLDLR-associated, | VLDLR, VLDLRCH |
| 224050 (3) | |
| Cerebral amyloid angiopathy, 105150 (3) | ABCA1, ABC1, HDLDT1, TGD |
| Cerebral amyloid angiopathy, 105150 (3) | CST3 |
| Cerebral arteriopathy with subcortical | NOTCH3, CADASIL, CASIL |
| infarcts and leukoencephalopathy, 125310 | |
| (3) | |
| Cerebral cavernous malformations-1, | CCM1, CAM, KRIT1 |
| 116860 (3) | |
| Cerebral cavernous malformations-2, | C7orf22, CCM2, MGC4067 |
| 603284 (3) | |
| Cerebral cavernous malformations 3, | PDCD10, TFAR15, CCM3 |
| 603285 (3) | |
| Cerebral dysgenesis, neuropathy, | SNAP29, CEDNIK |
| ichthyosis, and palmoplantar keratoderma | |
| syndrome, 609528 (3) | |
| Cerebrooculofacioskeletal syndrome, | ERCC2, EM9 |
| 214150 (3) | |
| Cerebrooculofacioskeletal syndrome, | ERCC5, XPG |
| 214150 (3) | |
| Cerebrooculofacioskeletal syndrome | ERCC6, CKN2, COFS, CSB |
| 214150 (3) | |
| Cerebrotendinous xanthomatosis, 213700 | CYP27A1, CYP27, CTX |
| (3) | |
| Cerebrovascular disease, occlusive (3) | SERPINA3, AACT, ACT |
| Ceroid lipofuscinosis, neuronal-1, infantile, | PPT1, CLN1 |
| 256730 (3) | |
| Ceroid-lipofuscinosis, neuronal 2, classic | CLN2 |
| late infantile, 204500 (3) | |
| Ceroid-lipofuscinosis, neuronal-3, juvenile, | CLN3, BTS |
| 204200 (3) | |
| Ceroid-lipofuscinosis, neuronal-5, variant | CLN5 |
| late infantile, 256731 (3) | |
| Ceroid-lipofuscinosis, neuronal-6, variant | CLN6 |
| late infantile, 601780 (3) | |
| Ceroid lipofuscinosis, neuronal 8, 600143 | CLN8, EPMR |
| (3) | |
| Ceroid lipofuscinosis, neuronal, variant | PPT1, CLN1 |
| juvenile type, with granular osmiophilic | |
| deposits (3) | |
| Cervical cancer, somatic, 603956 (3) | FGFR3, ACH |
| CETP deficiency, 607322 (3) | CETP |
| Chanarin-Dorfman syndrome, 275630 (3) | ABHD5, CGI58, IECN2, NCIE2 |
| Charcot-Marie-Tooth disease, axonal, type | HSPB1, HSP27, CMT2F |
| 2F, 606595 (3) | |
| Charcot-Marie-Tooth disease, dominant | MPZ, CMT1B, CMTDI3, CHM, DSS |
| intermediate 3, 607791 (3) | |
| Charcot-Marie-Tooth disease, dominant | DNM2 |
| intermediate B, 606482 (3) | |
| Charcot-Marie-Tooth disease, foot deformity | HOXD10, HOX4D |
| of (3) | |
| Charcot-Marie-Tooth disease, mixed axonal | GDAP1, CMT4A, CMT2K, CMT2G |
| and demyelinating type, 214400 (3) | |
| Charcot-Marie-Tooth disease, type 1A, | PMP22, CMT1A, CMT1E, DSS |
| 118220 (3) | |
| Charcot-Marie-Tooth disease, type 1B, | MPZ, CMT1B, CMTDI3, CHM, DSS |
| 118200 (3) | |
| Charcot-Marie-Tooth disease, type 1C, | LITAF, CMT1C |
| 601098 (3) | |
| Charcot-Marie-Tooth disease, type 1D, | EGR2, KROX20 |
| 607678 (3) | |
| Charcot-Marie-Tooth disease, type 1E, | PMP22, CMT1A, CMT1E, DSS |
| 118300 (3) | |
| Charcot-Marie-Tooth disease, type 1F, | NEFL, CMT2E, CMT1F |
| 607734 (3) | |
| Charcot-Marie-Tooth disease, type 2A1, | KIF1B, CMT2A, CMT2A1 |
| 118210 (3) | |
| Charcot-Marie-Tooth disease, type 2A2, | MFN2, KIAA0214, CMT2A2 |
| 609260 (3) | |
| Charcot-Marie-Tooth disease, type 2B, | RAB7, CMT2B, PSN |
| 600882 (3) | |
| Charcot-Marie-Tooth disease, type 2D, | GARS, SMAD1, CMT2D |
| 601472 (3) | |
| Charcot-Marie-Tooth disease, type 2E, | NEFL, CMT2E, CMT1F |
| 607684 (3) | |
| Charcot-Marie-Tooth disease, type 2G, | GDAP1, CMT4A, CMT2K, CMT2G |
| 607706 (3) | |
| Charcot-Marie-Tooth disease, type 2I, | MPZ, CMT1B, CMTDI3, CHM, DSS |
| 607677 (3) | |
| Charcot-Marie-Tooth disease, type 2J, | MPZ, CMT1B, CMTDI3, CHM, DSS |
| 607736 (3) | |
| Charcot-Marie-Tooth disease, type 2K, | GDAP1, CMT4A, CMT2K, CMT2G |
| 607831 (3) | |
| Charcot-Marie-Tooth disease, type 4A, | GDAP1, CMT4A, CMT2K, CMT2G |
| 214400 (3) | |
| Charcot-Marie-Tooth disease, type 4B1, | MTMR2, CMT4B1 |
| 601382 (3) | |
| Charcot-Marie-Tooth disease, type 4B2, | SBF2, MTMR13, CMT4B2 |
| 604563 (3) | |
| Charcot-Marie-Tooth disease, type 4B2, | SBF2, MTMR13, CMT4B2 |
| with early-onset glaucoma, 607739 (3) | |
| Charcot-Marie-Tooth disease, type 4C, | KIAA1985 |
| 601596 (3) | |
| Charcot-Marie-Tooth disease, type 4D, | NDRG1, HMSNL, CMT4D |
| 601455 (3) | |
| Charcot-Marie-Tooth neuropathy, X-linked | GJB1, CX32, CMTX1 |
| dominant, 1, 302800 (3) | |
| CHARGE syndrome, 214800 (3) | CHD7 |
| Char syndrome, 169100 (3) | TFAP2B, CHAR |
| Chediak-Higashi syndrome, 214500 (3) | CHS1, LYST |
| Cherubism, 118400 (3) | SH3BP2, CRPM |
| CHILD syndrome, 308050 (3) | NSDHL |
| Chitotriosidase deficiency (3) | CHIT |
| Chloride diarrhea, congenital, Finnish type, | SLC26A3, DRA, CLD |
| 214700 (3) | |
| Cholelithiasis, 600803 (3) | ABCB4, PGY3, MDR3 |
| Cholestasis, benign recurrent intrahepatic, | ATP8B1, FIC1, BRIC, PFIC1 |
| 243300 (3) | |
| Cholestasis, familial intrahepatic, of | ABCB4, PGY3, MDR3 |
| pregnancy, 147480 (3) | |
| Cholestasis, progressive familial | ATP8B1, FIC1, BRIC, PFIC1 |
| intrahepatic 1, 211600 (3) | |
| Cholestasis, progressive familial | ABCB11, BSEP, SPGP, PFIC2 |
| intrahepatic 2, 601847 (3) | |
| Cholestasis, progressive familial | ABCB4, PGY3, MDR3 |
| intrahepatic 3, 602347 (3) | |
| Cholestasis, progressive familial | HSD3B7, PFIC4 |
| intrahepatic 4, 607765 (3) | |
| Cholesteryl ester storage disease (3) | LIPA |
| Chondrocalcinosis 2, 118600 (3) | ANKH, HANK, ANK, CMDJ, CCAL2, |
| | CPPDD |
| Chondrodysplasia, Grebe type, 200700 (3) | GDF5, CDMP1 |
| Chondrodysplasia punctata, rhizomelic, type | GNPAT, DHAPAT |
| 2, 222765 (3) | |
| Chondrodysplasia punctata, X-linked | EBP, CDPX2, CPXD, CPX |
| dominant, 302960 (3) | |
| Chondrodysplasia punctata, X-linked | ARSE, CDPX1, CDPXR |
| recessive, 302950 (3) | |
| Chondrosarcoma, 215300 (3) | EXT1 |
| Chondrosarcoma, extraskeletal myxoid (3) | CSMF |
| Chondrosarcoma, extraskeletal myxoid (3) | EWSR1, EWS |
| Chorea, hereditary benign, 118700 (3) | TITF1, NKX2A, TTF1 |
| Choreoacanthocytosis, 200150 (3) | VPS13A, CHAC |
| Choreoathetosis, hypothyroidism, and | TITF1, NKX2A, TTF1 |
| respiratory distress (3) | |
| Choroideremia, 303100 (3) | CHM, TCD |
| Chromosome 22q13.3 deletion syndrome, | PSAP2, PROSAP2, KIAA1650 |
| 606232 (3) | |
| Chronic granulomatous disease, autosomal, | CYBA |
| due to deficiency of CYBA, 233690 (3) | |
| Chronic granulomatous disease due to | NCF1 |
| deficiency of NCF-1, 233700 (3) | |
| Chronic granulomatous disease due to | NCF2 |
| deficiency of NCF-2, 233710 (3) | |
| Chronic granulomatous disease, X-linked, | CYBB, CGD |
| 306400 (3) | |
| Chronic infections, due to opsonin defect (3) | MBL2, MBL, MBP1 |
| Chudley-Lowry syndrome, 309490 (3) | ATRX, XH2, XNP, MRXS3, SHS |
| Chylomicronemia syndrome, familial (3) | LPL, LIPD |
| Chylomicron retention disease, 246700 (3) | SARA2, SAR1B, CMRD |
| Chylomicron retention disease with | SARA2, SAR1B, CMRD |
| Marinesco-Sjogren syndrome, 607692 (3) | |
| Ciliary dyskinesia, primary, 1, 242650 (3) | DNAI1, CILD1, ICS, PCD |
| Ciliary dyskinesia, primary, 3 608644 (3) | DNAH5, HL1, PCD, CILD3 |
| CINCA syndrome, 607115 (3) | CIAS1, C1orf7, FCU, FCAS |
| Cirrhosis, cryptogenic (3) | KRT18 |
| Cirrhosis, cryptogenic (3) | KRT8 |
| Cirrhosis, noncryptogenic, susceptibility to, | KRT18 |
| 215600 (3) | |
| Cirrhosis, noncryptogenic, susceptibility to, | KRT8 |
| 215600 (3) | |
| Cirrhosis, North American Indian childhood | CIRH1A, NAIC, TEX292, KIAA1988 |
| type, 604901 (3) | |
| Citrullinemia, 215700 (3) | ASS |
| Citrullinemia, adult-onset type II, 603471 (3) | SLC25A13, CTLN2 |
| Citrullinemia, type II, neonatal-onset, | SLC25A13, CTLN2 |
| 605814 (3) | |
| Cleft lip/palate ectodermal dysplasia | HVEC, PVRL1, PVRR1, PRR1 |
| syndrome, 225000 (3) | |
| Cleft lip/palate, nonsyndromic, 608874 (3) | MSX1, HOX7, HYD1, OFC5 |
| Cleft palate with ankyloglossia, 303400 (3) | TBX22, CPX |
| Cleidocranial dysplasia, 119600 (3) | RUNX2, CBFA1, PEBP2A1, AML3 |
| Coats disease, 300216 (3) | NDP, ND |
| Cockayne syndrome, type A, 216400 (3) | ERCC8, CKN1, CSA |
| Cockayne syndrome, type B, 133540 (3) | ERCC6, CKN2, COFS, CSB |
| Codeine sensitivity (3) | CYP2D@, CYP2D, P450C2D |
| Coffin-Lowry syndrome, 303600 (3) | RPS6KA3, RSK2, MRX19 |
| Cohen syndrome, 216550 (3) | COH1 |
| Colchicine resistance (3) | ABCB1, PGY1, MDR1 |
| Cold-induced autoinflammatory syndrome, | CIAS1, C1orf7, FCU, FCAS |
| familial, 120100 (3) | |
| Cold-induced sweating syndrome, 272430 | CRLF1, CISS |
| (3) | |
| Coloboma, ocular, 120200 (3) | PAX6, AN2, MGDA |
| Coloboma, ocular, 120200 (3) | SHH, HPE3, HLP3, SMMCI |
| Colon adenocarcinoma (3) | RAD54B |
| Colon adenocarcinoma (3) | RAD54L, HR54, HRAD54 |
| Colon cancer (3) | BCL10 |
| Colon cancer (3) | PTPN12, PTPG1 |
| Colon cancer (3) | TGFBR2, HNPCC6 |
| Colon cancer, advanced (3) | SRC, ASV, SRC1 |
| Colon cancer, hereditary nonpolypopsis, | MLH3, HNPCC7 |
| type 7 (3) | |
| Colon cancer, somatic, 114500 (3) | PTPRJ, DEP1 |
| Colonic adenoma recurrence, reduced risk | ODC1 |
| of, 114500 (3) | |
| Colonic aganglionosis, total, with small | RET, MEN2A |
| bowel involvement (3) | |
| Colorblindness, deutan (3) | OPN1MW, GCP, CBD, CBBM |
| Colorblindness, protan (3) | OPN1LW, RCP, CBP, CBBM |
| Colorblindness, tritan (3) | OPN1SW, BCP, CBT |
| Colorectal adenomatous polyposis, | MUTYH |
| autosomal recessive, with pilomatricomas, | |
| 132600 (3) | |
| Colorectal cancer, 114500 (3) | AXIN2 |
| Colorectal cancer, 114500 (3) | BUB1B, BUBR1 |
| Colorectal cancer, 114500 (3) | EP300 |
| Colorectal cancer, 114500 (3) | PDGFRL, PDGRL, PRLTS |
| Colorectal cancer, 114500 (3) | PIK3CA |
| Colorectal cancer, 114500 (3) | TP53, P53, LFS1 |
| Colorectal cancer (3) | APC, GS, FPC |
| Colorectal cancer (3) | BAX |
| Colorectal cancer (3) | CTNNB1 |
| Colorectal cancer (3) | DCC |
| Colorectal cancer (3) | MCC |
| Colorectal cancer (3) | NRAS |
| Colorectal cancer, hereditary nonpolyposis, | MSH2, COCA1, FCC1, HNPCC1 |
| type 1, 120435 (3) | |
| Colorectal cancer, hereditary nonpolyposis, | MLH1, COCA2, HNPCC2 |
| type 2, 609310 (3) | |
| Colorectal cancer, hereditary nonpolyposis, | PMS1, PMSL1, HNPCC3 |
| type 3 (3) | |
| Colorectal cancer, hereditary nonpolyposis, | PMS2, PMSL2, HNPCC4 |
| type 4 (3) | |
| Colorectal cancer, hereditary nonpolyposis, | MSH6, GTBP, HNPCC5 |
| type 5 (3) | |
| Colorectal cancer, hereditary nonpolyposis, | TGFBR2, HNPCC6 |
| type 6 (3) | |
| Colorectal cancer, somatic, 109800 (3) | FGFR3, ACH |
| Colorectal cancer, somatic, 114500 (3) | FLCN, BHD |
| Colorectal cancer, somatic, 114500 (3) | MLH3, HNPCC7 |
| Colorectal cancer, somatic (3) | BRAF |
| Colorectal cancer, somatic (3) | DLC1 |
| Colorectal cancer, sporadic, 114500 (3) | PLA2G2A, PLA2B, PLA2L, MOM1 |
| Colorectal cancer, susceptibility to (3) | CCND1, PRAD1, BCL1 |
| Colorectal cancer with chromosomal | BUB1 |
| instability (3) | |
| Combined C6/C7 deficiency (3) | C6 |
| Combined factor V and VIII deficiency, | LMAN1, ERGIC53, F5F8D, MCFD1 |
| 227300 (3) | |
| Combined hyperlipemia, familial (3) | LPL, LIPD |
| Combined immunodeficiency, X-linked, | IL2RG, SCIDX1, SCIDX, IMD4 |
| moderate, 312863 (3) | |
| Combined oxidative phosphorylation | GFM1, EFG1, GFM |
| deficiency, 609060 (3) | |
| Combined SAP deficiency (3) | PSAP, SAP1 |
| Complex I, mitochondrial respiratory chain, | NDUFS6 |
| deficiency of, 252010 (3) | |
| Complex V, mitochondrial respiratory chain, | ATPAF2, ATP12 |
| deficiency of, 604273 (3) | |
| Cone dystrophy-1, 304020 (3) | RPGR, RP3, CRD, RP15, COD1 |
| Cone dystrophy-3, 602093 (3) | GUCA1A, GCAP |
| Cone-rod dystrophy, 300029 (3) | RPGR, RP3, CRD, RP15, COD1 |
| Cone-rod dystrophy 3 (3) | ABCA4, ABCR, STGD1, FFM, RP19 |
| Cone-rod dystrophy (3) | AIPL1, LCA4 |
| Cone-rod dystrophy 6, 601777(3) | GUCY2D, GUC2D, LCA1, CORD6 |
| Cone-rod dystrophy 9, 608194 (3) | RPGRIP1, LCA6, CORD9 |
| Cone-rod retinal dystrophy-2, 120970 (3) | CRX, CORD2, CRD |
| Congenital bilateral absence of vas | CFTR, ABCC7, CF, MRP7 |
| deferens, 277180 (3) | |
| Congenital cataracts, facial dysmorphism, | CTDP1, FCP1, CCFDN |
| and neuropathy, 604168 (3) | |
| Congenital disorder of glycosylation, type Ic, | ALG6 |
| 603147 (3) | |
| Congenital disorder of glycosylation, type Id, | ALG3, NOT56L, CDGS4 |
| 601110 (3) | |
| Congenital disorder of glycosylation, type Ie, | DPM1, MPDS, CDGIE |
| 608799 (3) | |
| Congenital disorder of glycosylation, type If, | MPDU1, SL15, CDGIF |
| 609180 (3) | |
| Congenital disorder of glycosylation, type Ig, | ALG12 |
| 607143 (3) | |
| Congenital disorder of glycosylation, type Ih, | ALG8 |
| 608104 (3) | |
| Congenital disorder of glycosylation, type Ii, | ALG2, CDGII |
| 607906 (3) | |
| Congenital disorder of glycosylation, type II, | DIBD1, ALG9 |
| 608776 (3) | |
| Congenital disorder of glycosylation, type | SLC35C1, FUCT1 |
| IIc, 266265 (3) | |
| Congenital disorder of glycosylation, type | B4GALT1, GGTB2, GT1, GTB |
| IId, 607091 (3) | |
| Congenital disorder of glycosylation, type | COG7, CDG2E |
| IIe, 608779 (3) | |
| Congenital disorder of glycosylation, type Ij, | DPAGT2, DGPT |
| 608093 (3) | |
| Congenital disorder of glycosylation, type Ik, | ALG1, HMAT1, HMT1 |
| 608540 (3) | |
| Congestive heart failure, susceptibility to (3) | ADRA2C, ADRA2L2 |
| Congestive heart failure, susceptibility to (3) | ADRB1, ADRB1R, RHR |
| Conjunctivitis, ligneous, 217090 (3) | PLG |
| Conotruncal anomaly face syndrome, | TBX1, DGS, CTHM, CAFS, TGA, |
| 217095 (3) | DORV, VCFS, DGCR |
| Contractural arachnodactyly, congenital (3) | FBN2, CCA |
| Convulsions, familial febrile, 4, 604352 (3) | MASS1, VLGR1, KIAA0686, FEB4, |
| | USH2C |
| COPD, rate of decline of lung function in, | MMP1, CLG |
| 606963 (3) | |
| Coproporphyria (3) | CPO |
| Corneal clouding, autosomal recessive (3) | APOA1 |
| Corneal dystrophy, Avellino type, 607541 | TGFBI, CSD2, CDGG1, CSD, BIGH3, |
| (3) | CDG2 |
| Corneal dystrophy, gelatinous drop-like, | TACSTD2, TROP2, M1S1 |
| 204870 (3) | |
| Corneal dystrophy, Groenouw type I, | TGFBI, CSD2, CDGG1, CSD, BIGH3, |
| 121900 (3) | CDG2 |
| Corneal dystrophy, hereditary polymorphous | VSX1, RINX, PPCD, PPD, KTCN |
| posterior, 122000 (3) | |
| Corneal dystrophy, hereditary polymorphous | COL8A2, FECD, PPCD2 |
| posterior, 2, 122000 (3) | |
| Corneal dystrophy, lattice type I, 122200 (3) | TGFBI, CSD2, CDGG1, CSD, BIGH3, |
| | CDG2 |
| Corneal dystrophy, lattice type IIIA, 608471 | TGFBI, CSD2, CDGG1, CSD, BIGH3, |
| (3) | CDG2 |
| Corneal dystrophy, Reis-Bucklers type, | TGFBI, CSD2, CDGG1, CSD, BIGH3, |
| 608470 (3) | CDG2 |
| Corneal dystrophy, Thiel-Behnke type, | TGFBI, CSD2, CDGG1, CSD, BIGH3, |
| 602082 (3) | CDG2 |
| Corneal fleck dystrophy, 121850 (3) | PIP5K3, CFD |
| Cornea plana congenita, recessive, 217300 | KERA, CNA2 |
| (3) | |
| Cornelia de Lange syndrome, 122470 (3) | NIPBL, CDLS |
| Coronary artery disease, autosomal | MEF2A, ADCAD1 |
| dominant, 1, 608320 (3) | |
| Coronary artery disease in familial | ABCA1, ABC1, HDLDT1, TGD |
| hypercholesterolemia, protection against, | |
| 143890 (3) | |
| Coronary artery disease, susceptibility to (3) | KL |
| Coronary artery disease, susceptibility to (3) | PON1, PON, ESA |
| Coronary artery disease, susceptibility to (3) | PON2 |
| Coronary artery spasm, susceptibility to (3) | PON1, PON, ESA |
| Coronary heart disease, susceptibility to (3) | MMP3, STMY1 |
| Coronary spasms, susceptibility to (3) | NOS3 |
| Corpus callosum, agenesis of, with mental | IGBP1 |
| retardation, ocular coloboma and | |
| micrognathia, 300472 (3) | |
| Cortisol resistance (3) | NR3C1, GCR, GRL |
| Cortisone reductase deficiency, 604931 (3) | GDH |
| Cortisone reductase deficiency, 604931 (3) | HSD11B1, HSD11, HSD11L |
| Costello syndrome, 218040 (3) | HRAS |
| Coumarin resistance, 122700 (3) | CYP2A6, CYP2A3, CYP2A, P450C2A |
| Cowden disease, 158350 (3) | PTEN, MMAC1 |
| Cowden-like syndrome, 158350 (3) | BMPR1A, ACVRLK3, ALK3 |
| CPT deficiency, hepatic, type IA, 255120 (3) | CPT1A |
| CPT deficiency, hepatic, type II, 600649 (3) | CPT2 |
| CPT II deficiency, lethal neonatal, 608836 | CPT2 |
| (3) | |
| Cramps, familial, potassium-aggravated (3) | SCN4A, HYPP, NAC1A |
| Craniofacial anomalies, empty sella turcica, | VSX1, RINX, PPCD, PPD, KTCN |
| corneal endothelial changes, and abnormal | |
| retinal and auditory bipolar cells (3) | |
| Craniofacial-deafness-hand syndrome, | PAX3, WS1, HUP2, CDHS |
| 122880 (3) | |
| Craniofacial-skeletal-dermatologic dysplasia | FGFR2, BEK, CFD1, JWS |
| (3) | |
| Craniofrontonasal dysplasia, 304110 (3) | EFNB1, EPLG2, CFNS, CFND |
| Craniometaphyseal dysplasia, 123000 (3) | ANKH, HANK, ANK, CMDJ, CCAL2, |
| | CPPDD |
| Craniosynostosis, nonspecific (3) | FGFR2, BEK, CFD1, JWS |
| Craniosynostosis, type 2, 604757 (3) | MSX2, CRS2, HOX8 |
| CRASH syndrome, 303350 (3) | L1CAM, CAML1, HSAS1 |
| Creatine deficiency syndrome, X-linked, | SLC6A8, CRTR |
| 300352 (3) | |
| Creatine phosphokinase, elevated serum, | CAV3, LGMD1C |
| 123320 (3) | |
| Creatine phosphokinase, elevated serum, | CAV3, LGMD1C |
| 123320 (3) | |
| Creutzfeldt-Jakob disease, 123400 (3) | PRNP, PRIP |
| Creutzfeldt-Jakob disease, variant, | HLA-DQB1 |
| resistance to, 123400 (3) | |
| Crigler-Najjar syndrome, type I, 218800 (3) | UGT1A1, UGT1, GNT1 |
| Crigler-Najjar syndrome, type II, 606785 (3) | UGT1A1, UGT1, GNT1 |
| Crohn disease, susceptibility to, 266600 (3) | CARD15, NOD2, IBD1, CD, ACUG, |
| | PSORAS1 |
| Crohn disease, susceptibility to, 266600 (3) | DLG5, PDLG, KIAA0583 |
| Crouzon syndrome, 123500 (3) | FGFR2, BEK, CFD1, JWS |
| Crouzon syndrome with acanthosis | FGFR3, ACH |
| nigricans (3) | |
| Cryptorchidism, bilateral, 219050 (3) | LGR8, GREAT |
| Cryptorchidism, idiopathic, 219050 (3) | INSL3 |
| Currarino syndrome, 176450 (3) | HLXB9, HOXHB9, SCRA1 |
| Cutis laxa, AD, 123700 (3) | ELN |
| Cutis laxa, autosomal dominant, 123700 (3) | FBLN5, ARMD3 |
| Cutis laxa, autosomal recessive, 219100 (3) | FBLN5, ARMD3 |
| Cutis laxa, neonatal (3) | ATP7A, MNK, MK, OHS |
| Cyclic ichthyosis with epidermolytic | KRT1 |
| hyperkeratosis, 607602 (3) | |
| Cylindromatosis, familial, 132700 (3) | CYLD1, CDMT, EAC |
| Cystathioninuria, 219500 (3) | CTH |
| Cystic fibrosis, 219700 (3) | CFTR, ABCC7, CF, MRP7 |
| Cystinosis, atypical nephropathic (3) | CTNS |
| Cystinosis, late-onset juvenile or adolescent | CTNS |
| nephropathic, 219900 (3) | |
| Cystinosis, nephropathic, 219800 (3) | CTNS |
| Cystinosis, ocular nonnephropathic, 219750 | CTNS |
| (3) | |
| Cystinuria, 220100 (3) | SLC3A1, ATR1, D2H, NBAT |
| Cystinuria, type II (3) | SLC7A9, CSNU3 |
| Cystinuria, type III (3) | SLC7A9, CSNU3 |
| D-2-hydroxyglutaric aciduria, 600721 (3) | D2HGD |
| Darier disease, 124200 (3) | ATP2A2, ATP2B, DAR |
| D-bifunctional protein deficiency, 261515 (3) | HSD17B4 |
| Deafness, autosomal dominant 10, 601316 | EYA4, DFNA10, CMD1J |
| (3) | |
| Deafness, autosomal dominant 1, 124900 | DIAPH1, DFNA1, LFHL1 |
| (3) | |
| Deafness, autosomal dominant 11, | MYO7A, USH1B, DFNB2, DFNA11 |
| neurosensory, 601317 (3) | |
| Deafness, autosomal dominant 12, 601842 | TECTA, DFNA8, DFNA12, DFNB21 |
| (3) | |
| Deafness, autosomal dominant 13, 601868 | COL11A2, STL3, DFNA13 |
| (3) | |
| Deafness, autosomal dominant 15, 602459 | POU4F3, BRN3C |
| (3) | |
| Deafness, autosomal dominant 17, 603622 | MYH9, MHA, FTNS, DFNA17 |
| (3) | |
| Deafness, autosomal dominant 20/26, | ACTG1, DFNA20, DFNA26 |
| 604717 (3) | |
| Deafness, autosomal dominant 22, 606346 | MYO6, DFNA22, DFNB37 |
| (3) | |
| Deafness, autosomal dominant 2, 600101 | GJB3, CX31, DFNA2 |
| (3) | |
| Deafness, autosomal dominant 2, 600101 | KCNQ4, DFNA2 |
| (3) | |
| Deafness, autosomal dominant 28, 608641 | TFCP2L3, DFNA28 |
| (3) | |
| Deafness, autosomal dominant 3, 601544 | GJB2, CX26, DFNB1, PPK, DFNA3, |
| (3) | KID, HID |
| Deafness, autosomal dominant 3, 601544 | GJB6, CX30, DFNA3, HED, ED2 |
| (3) | |
| Deafness, autosomal dominant 36, 606705 | TMC1, DFNB7, DFNB11, DFNA36 |
| (3) | |
| Deafness, autosomal dominant 36, with | DSPP, DPP, DGI1, DFNA39, DTDP2 |
| dentinogenesis, 605594 (3) | |
| Deafness, autosomal dominant 40 (3) | CRYM, DFNA40 |
| Deafness, autosomal dominant 4, 600652 | MYH14, KIAA2034, DFNA4 |
| (3) | |
| Deafness, autosomal dominant 5 (3) | DFNA5 |
| Deafness, autosomal dominant 8, 601543 | TECTA, DFNA8, DFNA12, DFNB21 |
| (3) | |
| Deafness, autosomal dominant 9, 601369 | COCH, DFNA9 |
| (3) | |
| Deafness, autosomal dominant | MYO1A |
| nonsyndromic sensorineural, 607841 (3) | |
| Deafness, autosomal dominant, with | GJB3, CX31, DFNA2 |
| peripheral neuropathy (3) | |
| Deafness, autosomal recessive 10, | TMPRSS3, ECHOS1, DFNB8, DFNB10 |
| congenital, 605316 (3) | |
| Deafness, autosomal recessive 1, 220290 | GJB2, CX26, DFNB1, PPK, DFNA3, |
| (3) | KID, HID |
| Deafness, autosomal recessive 12, 601386 | CDH23, USH1D |
| (3) | |
| Deafness, autosomal recessive 12, modifier | ATP2B2, PMCA2 |
| of, 601386 (3) | |
| Deafness, autosomal recessive 16, 603720 | STRC, DFNB16 |
| (3) | |
| Deafness, autosomal recessive 18, 602092 | USH1C, DFNB18 |
| (3) | |
| Deafness, autosomal recessive 21, 603629 | TECTA, DFNA8, DFNA12, DFNB21 |
| (3) | |
| Deafness, autosomal recessive 22, 607039 | OTOA, DFNB22 |
| (3) | |
| Deafness, autosomal recessive 23, 609533 | PCDH15, DFNB23 |
| (3) | |
| Deafness, autosomal recessive 29 (3) | CLDN14, DFNB29 |
| Deafness, autosomal recessive 2, | MYO7A, USH1B, DFNB2, DFNA11 |
| neurosensory, 600060 (3) | |
| Deafness, autosomal recessive 30, 607101 | MYO3A, DFNB30 |
| (3) | |
| Deafness, autosomal recessive 31, 607084 | WHRN, CIP98, KIAA1526, DFNB31 |
| (3) | |
| Deafness, autosomal recessive 3, 600316 | MYO15A, DFNB3 |
| (3) | |
| Deafness, autosomal recessive 36, 609006 | ESPN |
| (3) | |
| Deafness, autosomal recessive 37, 607821 | MYO6, DFNA22, DFNB37 |
| (3) | |
| Deafness, autosomal recessive (3) | GJB3, CX31, DFNA2 |
| Deafness, autosomal recessive 4, 600791 | SLC26A4, PDS, DFNB4 |
| (3) | |
| Deafness, autosomal recessive 61 (3) | PRES, DFNB61, SLC26A5 |
| Deafness, autosomal recessive 6, 600971 | TMIE, DFNB6 |
| (3) | |
| Deafness, autosomal recessive 7, 600974 | TMC1, DFNB7, DFNB11, DFNA36 |
| (3) | |
| Deafness, autosomal recessive 8, childhood | TMPRSS3, ECHOS1, DFNB8, DFNB10 |
| onset, 601072 (3) | |
| Deafness, autosomal recessive 9, 601071 | OTOF, DFNB9, NSRD9 |
| (3) | |
| Deafness, congenital heart defects, and | JAG1, AGS, AHD |
| posterior embryotoxon (3) | |
| Deafness, nonsyndromic (3) ( ) | KIAA1199 |
| Deafness, nonsyndromic neurosensory, | GJB6, CX30, DFNA3, HED, ED2 |
| digenic (3) | |
| Deafness, sensorineural, with hypertrophic | MYO6, DFNA22, DFNB37 |
| cardiomyopathy, 606346 (3) | |
| Deafness, X-linked 1, progressive (3) | TIMM8A, DFN1, DDP, MTS, DDP1 |
| Deafness, X-linked 3, conductive, with | POU3F4, DFN3 |
| stapes fixation, 304400 (3) | |
| Debrisoquine sensitivity (3) | CYP2D@, CYP2D, P450C2D |
| Dejerine-Sottas disease, 145900 (3) | PMP22, CMT1A, CMT1E, DSS |
| Dejerine-Sottas neuropathy, 145900 (3) | EGR2, KROX20 |
| Dejerine-Sottas neuropathy, autosomal | PRX, CMT4F |
| recessive, 145900 (3) | |
| Dejerine-Sottas syndrome, 145900 (3) | MPZ, CMT1B, CMTDI3, CHM, DSS |
| Delayed sleep phase syndrome, | AANAT, SNAT |
| susceptibility to (3) | |
| Dementia, familial British, 176500 (3) | ITM2B, BRI, ABRI, FBD |
| Dementia, familial Danish, 117300 (3) | ITM2B, BRI, ABRI, FBD |
| Dementia, frontotemporal, 600274 (3) | PSEN1, AD3 |
| Dementia, frontotemporal, with | MAPT, MTBT1, DDPAC, MSTD |
| parkinsonism, 600274 (3) | |
| Dementia, Lewy body, 127750 (3) | SNCA, NACP, PARK1, PARK4 |
| Dementia, Lewy body, 127750 (3) | SNCB |
| Dementia, Pick disease-like, 172700 (3) | MAPT, MTBT1, DDPAC, MSTD |
| Dementia, vascular, susceptibility to (3) | TNF, TNFA |
| Dengue fever, protection against (3) | CD209, CDSIGN |
| Dental anomalies, isolated (3) | RUNX2, CBFA1, PEBP2A1, AML3 |
| Dentatorubro-pallidoluysian atrophy, 125370 | DRPLA |
| (3) | |
| Dent disease, 300009 (3) | CLCN5, CLCK2, NPHL2, DENTS |
| Dentin dysplasia, type II, 125420 (3) | DSPP, DPP, DGI1, DFNA39, DTDP2 |
| Dentinogenesis imperfecta, Shields type II, | DSPP, DPP, DGI1, DFNA39, DTDP2 |
| 125490 (3) | |
| Dentinogenesis imperfecta, Shields type III, | DSPP, DPP, DGI1, DFNA39, DTDP2 |
| 125500 (3) | |
| Dent syndrome, 300009 (3) | OCRL, LOCR, OCRL1, NPHL2 |
| Denys-Drash syndrome, 194080 (3) | WT1 |
| Dermatofibrosarcoma protuberans (3) | PDGFB, SIS |
| De Sanctis-Cacchione syndrome, 278800 | ERCC6, CKN2, COFS, CSB |
| (3) | |
| Desmoid disease, hereditary, 135290 (3) | APC, GS, FPC |
| Desmosterolosis, 602398 (3) | DHCR24, KIAA0018 |
| Diabetes insipidus, nephrogenic, 304800 (3) | AVPR2, DIR, DI1, ADHR |
| Diabetes insipidus, nephrogenic, autosomal | AQP2 |
| dominant, 125800 (3) | |
| Diabetes insipidus, nephrogenic, autosomal | AQP2 |
| recessive, 222000 (3) | |
| Diabetes insipidus, neurohypophyseal, | AVP, AVRP, VP |
| 125700 (3) | |
| Diabetes mellitus, 125853 (3) | ABCC8, SUR, PHHI, SUR1 |
| Diabetes mellitus, insulin-dependent, | TCF1, HNF1A, MODY3 |
| 222100 (3) | |
| Diabetes mellitus, insulin-dependent, 5, | SUMO4, IDDM5 |
| 600320 (3) | |
| Diabetes mellitus, insulin-dependent, | PTPN8, PEP, PTPN22, LYP |
| susceptibility to, 222100 (3) | |
| Diabetes mellitus, insulin-resistant, with | INSR |
| acanthosis nigricans (3) | |
| Diabetes mellitus, insulin-resistant, with | PPARG, PPARG1, PPARG2 |
| acanthosis nigricans and hypertension, | |
| 604367 (3) | |
| Diabetes mellitus, neonatal-onset, 606176 | GCK |
| (3) | |
| Diabetes mellitus, noninsulin-dependent, | GCGR |
| 125853 (3) | |
| Diabetes mellitus, noninsulin-dependent, | GPD2 |
| 125853 (3) | |
| Diabetes mellitus, noninsulin-dependent, | HNF4A, TCF14, MODY1 |
| 125853 (3) | |
| Diabetes mellitus, noninsulin-dependent, | IRS2 |
| 125853 (3) | |
| Diabetes mellitus, noninsulin-dependent, | MAPK8IP1, IB1 |
| 125853 (3) | |
| Diabetes mellitus, noninsulin-dependent, | NEUROD1, NIDDM |
| 125853 (3) | |
| Diabetes mellitus, noninsulin-dependent, | TCF2, HNF2 |
| 125853 (3) | |
| Diabetes mellitus, noninsulin-dependent, 2, | TCF1, HNF1A, MODY3 |
| 125853 (3) | |
| Diabetes mellitus, noninsulin-dependent (3) | IRS1 |
| Diabetes mellitus, noninsulin-dependent (3) | SLC2A2, GLUT2 |
| Diabetes mellitus, noninsulin-dependent (3) | SLC2A4, GLUT4 |
| Diabetes mellitus, noninsulin-dependent, | CAPN10 |
| 601283 (3) | |
| Diabetes mellitus, non-insulin-dependent, | ENPP1, PDNP1, NPPS, M6S1, PCA1 |
| susceptibility to, 125853 (3) | |
| Diabetes mellitus, noninsulin-dependent, | RETN, RSTN, FIZZ3 |
| susceptibility to, 125853 (3) | |
| Diabetes mellitus, permanent neonatal, with | PTF1A |
| cerebellar agenesis, 609069 (3) | |
| Diabetes mellitus, permanent neonatal, with | KCNJ11, BIR, PHHI |
| neurologic features, 606176 (3) | |
| Diabetes mellitus, type II, 125853 (3) | AKT2 |
| Diabetes mellitus, type II, susceptibility to, | IPF1 |
| 125853 (3) | |
| Diabetes mellitus, type I, susceptibility to, | FOXP3, IPEX, AIID, XPID, PIDX |
| 222100 (3) | |
| Diabetes, permanent neonatal, 606176 (3) | KCNJ11, BIR, PHHI |
| Diabetic nephropathy, susceptibility to, | ACE, DCP1, ACE1 |
| 603933 (3) | |
| Diabetic retinopathy, NIDDM-related, | VEGF |
| susceptibility to, 125853 (3) | |
| Diastrophic dysplasia, 222600 (3) | SLC26A2, DTD, DTDST, D5S1708, |
| | EDM4 |
| Diastrophic dysplasia, broad bone- | SLC26A2, DTD, DTDST, D5S1708, |
| platyspondylic variant (3) | EDM4 |
| DiGeorge syndrome, 188400 (3) | TBX1, DGS, CTHM, CAFS, TGA, |
| | DORV, VCFS, DGCR |
| Dihydropyrimidinuria (3) | DPYS, DHP |
| Dilated cardiomyopathy with woolly hair and | DSP, KPPS2, PPKS2 |
| keratoderma, 605676 (3) | |
| Dimethylglycine dehydrogenase deficiency, | DMGDH, DMGDHD |
| 605850 (3) | |
| Disordered steroidogenesis, isolated (3) | POR |
| Dissection of cervical arteries (3) | COL1A1 |
| DNA ligase I deficiency (3) | LIG1 |
| DNA topoisomerase I, camptothecin- | TOP1 |
| resistant (3) | |
| DNA topoisomerase II, resistance to | TOP2A, TOP2 |
| inhibition of, by amsacrine (3) | |
| Dopamine-beta-hydroxylase activity levels, | DBH |
| plasma (3) | |
| Dopamine beta-hydroxylase deficiency, | DBH |
| 223360 (3) | |
| Dosage-sensitive sex reversal, 300018 (3) | DAX1, AHC, AHX, NROB1 |
| Double-outlet right ventricle, 217095 (3) | CFC1, CRYPTIC, HTX2 |
| Down syndrome, risk of, 190685 (3) | MTR |
| Doyne honeycomb degeneration of retina, | EFEMP1, FBNL, DHRD |
| 126600 (3) | |
| Drug addiction, susceptibility to (3) | FAAH |
| Duane-radial ray syndrome, 607323 (3) | SALL4, HSAL4 |
| Dubin-Johnson syndrome, 237500 (3) | ABCC2, CMOAT |
| Duchenne muscular dystrophy, 310200 (3) | DMD, BMD |
| Dyggve-Melchior-Clausen disease, 223800 | DYM, FLJ90130, DMC, SMC |
| (3) | |
| Dysalbuminemic hyperthyroxinemia (3) | ALB |
| Dysautonomia, familial, 223900 (3) | IKBKAP, IKAP |
| Dyschromatosis symmetrica hereditaria, | ADAR, DRADA, DSH, DSRAD |
| 127400 (3) | |
| Dyserythropoietic anemia with | GATA1, GF1, ERYF1, NFE1 |
| thrombocytopenia, 300367 (3) | |
| Dysfibrinogenemia, alpha type, causing | FGA |
| bleeding diathesis (3) | |
| Dysfibrinogenemia, alpha type, causing | FGA |
| recurrent thrombosis (3) | |
| Dysfibrinogenemia, beta type (3) | FGB |
| Dysfibrinogenemia, gamma type (3) | FGG |
| Dyskeratosis congenita-1, 305000 (3) | DKC1, DKC |
| Dyskeratosis congenita, autosomal | TERC, TRC3, TR |
| dominant, 127550 (3) | |
| Dyslexia, susceptibility to, 1, 127700 (3) | DYX1C1, DYXC1, DYX1 |
| Dyslexia, susceptibility to, 2, 600202 (3) | KIAA0319, DYX2, DYLX2, DLX2 |
| Dysprothrombinemia (3) | F2 |
| Dyssegmental dysplasia, Silverman- | HSPG2, PLC, SJS, SJA, SJS1 |
| Handmaker type, 224410 (3) | |
| Dystonia-12, 128235 (3) | ATP1A3, DYT12, RDP |
| Dystonia-1, torsion, 128100 (3) | DYT1, TOR1A |
| Dystonia, DOPA-responsive, 128230 (3) | GCH1, DYT5 |
| Dystonia, early-onset atypical, with | DYT1, TOR1A |
| myoclonic features (3) | |
| Dystonia, myoclonic, 159900 (3) | DRD2 |
| Dystonia, myoclonic, 159900 (3) | SGCE, DYT11 |
| Dystonia, primary cervical (3) | DRD5, DRD1B, DRD1L2 |
| Dystransthyretinemic hyperthyroxinemia(3) | TTR, PALB |
| EBD, Bart type, 132000 (3) | COL7A1 |
| EBD, localisata variant (3) | COL7A1 |
| Ectodermal dysplasia-1, anhidrotic, 305100 | ED1, EDA, HED |
| (3) | |
| Ectodermal dysplasia 2, hidrotic, 129500 (3) | GJB6, CX30, DFNA3, HED, ED2 |
| Ectodermal dysplasia, anhidrotic, 224900 | EDARADD |
| (3) | |
| Ectodermal dysplasia, anhidrotic, | IKBKG, NEMO, FIP3, IP2 |
| lymphedema and immunodeficiency, 300301 | |
| (3) | |
| Ectodermal dysplasia, anhidrotic, with T-cell | NFKBIA, IKBA |
| immunodeficiency (3) | |
| Ectodermal dysplasia, hypohidrotic, | EDAR, DL, ED3, EDA3 |
| autosomal dominant, 129490 (3) | |
| Ectodermal dysplasia, hypohidrotic, | EDAR, DL, ED3, EDA3 |
| autosomal recessive, 224900 (3) | |
| Ectodermal dysplasia, hypohidrotic, with | IKBKG, NEMO, FIP3, IP2 |
| immune deficiency, 300291 (3) | |
| Ectodermal dysplasia, Margarita Island type, | HVEC, PVRL1, PVRR1, PRR1 |
| 225060 (3) | |
| Ectodermal dysplasia/skin fragility | PKP1 |
| syndrome, 604536 (3) | |
| Ectopia lentis, familial, 129600 (3) | FBN1, MFS1, WMS |
| Ectopia pupillae, 129750 (3) | PAX6, AN2, MGDA |
| Ectrodactyly, ectodermal dysplasia, and | TP73L, TP63, KET, EEC3, SHFM4, |
| cleft lip/palate syndrome 3, 604292 (3) | LMS, RHS |
| Ehlers-Danlos due to tenascin X deficiency, | TNXB, TNX, TNXB1, TNXBS, TNXB2 |
| 606408 (3) | |
| Ehlers-Danlos syndrome, hypermobility | TNXB, TNX, TNXB1, TNXBS, TNXB2 |
| type, 130020 (3) | |
| Ehlers-Danlos syndrome, progeroid form, | B4GALT7, XGALT1, XGPT1 |
| 130070 (3) | |
| Ehlers-Danlos syndrome, type I, 130000 (3) | COL1A1 |
| Ehlers-Danlos syndrome, type I, 130000 (3) | COL5A1 |
| Ehlers-Danlos syndrome, type I, 130000 (3) | COL5A2 |
| Ehlers-Danlos syndrome, type II, 130010 (3) | COL5A1 |
| Ehlers-Danlos syndrome, type III, 130020 | COL3A1 |
| (3) | |
| Ehlers-Danlos syndrome, type IV, 130050 | COL3A1 |
| (3) | |
| Ehlers-Danlos syndrome, type VI, 225400 | PLOD, PLOD1 |
| (3) | |
| Ehlers-Danlos syndrome, type VII, 130060 | COL1A1 |
| (3) | |
| Ehlers-Danlos syndrome, type VIIA2, | COL1A2 |
| 130060 (3) | |
| Ehlers-Danlos syndrome, type VIIC, 225410 | ADAMTS2, NPI |
| (3) | |
| Elite sprint athletic performance (3) | ACTN3 |
| Elliptocytosis-1 (3) | EPB41, EL1 |
| Elliptocytosis-2 (3) | SPTA1 |
| Elliptocytosis-3 (3) | SPTB |
| Elliptocytosis, Malaysian-Melanesian type | SLC4A1, AE1, EPB3 |
| (3) | |
| Ellis-van Creveld syndrome, 225500 (3) | EVC |
| Ellis-van Creveld syndrome, 225500 (3) | LBN, EVC2 |
| Emery-Dreifuss muscular dystrophy, | EMD, EDMD, STA |
| 310300 (3) | |
| Emery-Dreifuss muscular dystrophy, AD, | LMNA, LMN1, EMD2, FPLD, CMD1A, |
| 181350 (3) | HGPS, LGMD1B |
| Emery-Dreifuss muscular dystrophy, AR, | LMNA, LMN1, EMD2, FPLD, CMD1A, |
| 604929 (3) | HGPS, LGMD1B |
| Emphysema (3) | PI, AAT |
| Emphysema-cirrhosis (3) | PI, AAT |
| Encephalopathy, familial, with neuroserpin | SERPINI1, PI12 |
| inclusion bodies, 604218 (3) | |
| Encephalopathy, progressive mitochondrial, | COX10 |
| with proximal renal tubulopathy due to | |
| cytochrome c oxidase deficiency (3) | |
| Enchondromatosis, Ollier type, 166000 (3) | PTHR1, PTHR |
| Endometrial carcinoma (3) | CDH1, UVO |
| Endometrial carcinoma (3) | MSH3 |
| Endometrial carcinoma (3) | MSH6, GTBP, HNPCC5 |
| Endometrial carcinoma (3) | PTEN, MMAC1 |
| Endotoxin hyporesponsiveness (3) | TLR4 |
| Endplate acetylcholinesterase deficiency, | COLQ, EAD |
| 603034 (3) | |
| Enhanced S-cone syndrome, 268100 (3) | NR2E3, PNR, ESCS |
| Enlarged vestibular aqueduct, 603545 (3) | SLC26A4, PDS, DFNB4 |
| Enolase-beta deficiency (3) | ENO3 |
| Enterokinase deficiency, 226200 (3) | PRSS7, ENTK |
| Eosinophil peroxidase deficiency, 261500 | EPX |
| (3) | |
| Epidermodysplasia verruciformis, 226400 | EVER1, EV1 |
| (3) | |
| Epidermodysplasia verruciformis, 226400 | EVER2, EV2 |
| (3) | |
| Epidermolysis bullosa dystrophica, AD, | COL7A1 |
| 131750 (3) | |
| Epidermolysis bullosa dystrophica, AR, | COL7A1 |
| 226600 (3) | |
| Epidermolysis bullosa, generalized atrophic | COL17A1, BPAG2 |
| benign, 226650 (3) | |
| Epidermolysis bullosa, generalized atrophic | ITGB4 |
| benign, 226650 (3) | |
| Epidermolysis bullosa, generalized atrophic | LAMA3, LOCS |
| benign, 226650 (3) | |
| Epidermolysis bullosa, generalized atrophic | LAMB3 |
| benign, 226650 (3) | |
| Epidermolysis bullosa, generalized atrophic | LAMC2, LAMNB2, LAMB2T |
| benign, 226650 (3) | |
| Epidermolysis bullosa, Herlitz junctional | LAMB3 |
| type, 226700 (3) | |
| Epidermolysis bullosa, Herlitz junctional | LAMC2, LAMNB2, LAMB2T |
| type, 226700 (3) | |
| Epidermolysis bullosa, junctional, Herlitz | LAMA3, LOCS |
| type, 226700 (3) | |
| Epidermolysis bullosa, junctional, with | ITGB4 |
| pyloric atresia, 226730 (3) | |
| Epidermolysis bullosa, junctional, with | ITGA6 |
| pyloric stenosis, 226730 (3) | |
| Epidermolysis bullosa, lethal acantholytic, | DSP, KPPS2, PPKS2 |
| 609638 (3) | |
| Epidermolysis bullosa of hands and feet, | ITGB4 |
| 131800 (3) | |
| Epidermolysis bullosa, pretibial, 131850 (3) | COL7A1 |
| Epidermolysis bullosa pruriginosa, 604129 | COL7A1 |
| (3) | |
| Epidermolysis bullosa simplex, Koebner, | KRT14 |
| Dowling-Meara, and Weber-Cockayne types, | |
| 131900, 131760, 131800 (3) | |
| Epidermolysis bullosa simplex, Koebner, | KRT5 |
| Dowling-Meara, and Weber-Cockayne types, | |
| 131900, 131760, 131800 (3) | |
| Epidermolysis bullosa simplex, Ogna type, | PLEC1, PLTN, EBS1 |
| 131950 (3) | |
| Epidermolysis bullosa simplex, recessive, | KRT14 |
| 601001 (3) | |
| Epidermolysis bullosa simplex with mottled | KRT5 |
| pigmentation, 131960 (3) | |
| Epidermolytic hyperkeratosis, 113800 (3) | KRT10 |
| Epidermolytic hyperkeratosis, 113800 (3) | KRT1 |
| Epidermolytic palmoplantar keratoderma, | KRT9, EPPK |
| 144200 (3) | |
| Epilepsy, benign, neonatal, type 1, 121200 | KCNQ2, EBN1 |
| (3) | |
| Epilepsy, benign neonatal, type 2, 121201 | KCNQ3, EBN2, BFNC2 |
| (3) | |
| Epilepsy, childhood absence, 607681 (3) | GABRG2, GEFSP3, CAE2, ECA2 |
| Epilepsy, childhood absence, 607682 (3) | CLCN2, EGMA, ECA3, EGI3 |
| Epilepsy, childhood absence, evolving to | JRK, JH8 |
| juvenile myoclonic epilepsy (3) | |
| Epilepsy, generalized idiopathic, 600669 (3) | CACNB4, EJM |
| Epilepsy, generalized, with febrile seizures | GABRG2, GEFSP3, CAE2, ECA2 |
| plus, 604233 (3) | |
| Epilepsy, generalized, with febrile seizures | SCN1A, GEFSP2, SMEI |
| plus, type 2, 604233 (3) | |
| Epilepsy, idopathic generalized, | ME2 |
| susceptibility to, 600669 (3) | |
| Epilepsy, juvenile absence, 607631 (3) | CLCN2, EGMA, ECA3, EGI3 |
| Epilepsy, juvenile myoclonic, 606904 (3) | CACNB4, EJM |
| Epilepsy, juvenile myoclonic, 606904 (3) | CLCN2, EGMA, ECA3, EGI3 |
| Epilepsy, juvenile myoclonic, 606904 (3) | GABRA1, EJM |
| Epilepsy, myoclonic, Lafora type, 254780 | EPM2A, MELF, EPM2 |
| (3) | |
| Epilepsy, myoclonic, Lafora type, 254780 | NHLRC1, EPM2A, EPM2B |
| (3) | |
| Epilepsy, neonatal myoclonic, with | SLC25A22, GC1 |
| suppression-burst pattern, 609304 (3) | |
| Epilepsy, nocturnal frontal lobe, 1, 600513 | CHRNA4, ENFL1 |
| (3) | |
| Epilepsy, nocturnal frontal lobe, 3, 605375 | CHRNB2, EFNL3 |
| (3) | |
| Epilepsy, partial, with auditory features, | LGI1, EPT, ETL1 |
| 600512 (3) | |
| Epilepsy, progressive myoclonic 1, 254800 | CSTB, STFB, EPM1 |
| (3) | |
| Epilepsy, progressive myoclonic 2B, 254780 | NHLRC1, EPM2A, EPM2B |
| (3) | |
| Epilepsy, severe myoclonic, of infancy, | SCN1A, GEFSP2, SMEI |
| 607208 (3) | |
| Epilepsy with grand mal seizures on | CLCN2, EGMA, ECA3, EGI3 |
| awakening, 607628 (3) | |
| Epilepsy, X-linked, with variable learning | SYN1 |
| disabilities and behavior disorders, 300491 | |
| (3) | |
| Epiphyseal dysplasia, multiple 1, 132400 (3) | COMP, EDM1, MED, PSACH |
| Epiphyseal dysplasia, multiple, 226900 (3) | SLC26A2, DTD, DTDST, D5S1708, |
| | EDM4 |
| Epiphyseal dysplasia, multiple, 3, 600969 | COL9A3, EDM3, IDD |
| (3) | |
| Epiphyseal dysplasia, multiple, 5, 607078 | MATN3, EDM5, HOA |
| (3) | |
| Epiphyseal dysplasia, multiple, COL9A1- | COL9A1, MED |
| related (3) | |
| Epiphyseal dysplasia, multiple, type 2, | COL9A2, EDM2 |
| 600204 (3) | |
| Epiphyseal dysplasia, multiple, with | COL9A3, EDM3, IDD |
| myopathy (3) | |
| Episodic ataxia/myokymia syndrome, | KCNA1, AEMK, EA1 |
| 160120 (3) | |
| Episodic ataxia, type 2, 108500 (3) | CACNA1A, CACNL1A4, SCA6 |
| Epithelial ovarian cancer, somatic, 604370 | OPCML |
| (3) | |
| Epstein syndrome, 153650 (3) | MYH9, MHA, FTNS, DFNA17 |
| Erythermalgia, primary, 133020 (3) | SCN9A, NENA, PN1 |
| Erythremias, alpha-(3) | HBA1 |
| Erythremias, beta-(3) | HBB |
| Erythrocytosis (3) | HBA2 |
| Erythrocytosis, familial, 133100 (3) | EPOR |
| Erythrokeratoderma, progressive symmetric, | LOR |
| 602036 (3) | |
| Erythrokeratodermia variabilis, 133200 (3) | GJB3, CX31, DFNA2 |
| Erythrokeratodermia variabilis with | GJB4, CX30.3 |
| erythema gyratum repens, 133200 (3) | |
| Esophageal cancer, 133239 (3) | TGFBR2, HNPCC6 |
| Esophageal carcinoma, somatic, 133239 (3) | RNF6 |
| Esophageal squamous cell carcinoma, | LZTS1, F37, FEZ1 |
| 133239 (3) | |
| Esophageal squamous cell carcinoma, | WWOX, FOR |
| 133239 (3) | |
| Estrogen resistance (3) | ESR1, ESR |
| Ethylmalonic encephalopathy, 602473 (3) | ETHE1, HSCO, D83198 |
| Ewing sarcoma (3) | EWSR1, EWS |
| Exertional myoglobinuria due to deficiency | LDHA, LDH1 |
| of LDH-A (3) | |
| Exostoses, multiple, type 1, 133700 (3) | EXT1 |
| Exostoses, multiple, type 2, 133701 (3) | EXT2 |
| Exudative vitreoretinopathy, 133780 (3) | FZD4, EVR1 |
| Exudative vitreoretinopathy, dominant, | LRP5, BMND1, LRP7, LR3, OPPG, |
| 133780 (3) | VBCH2 |
| Exudative vitreoretinopathy, recessive, | LRP5, BMND1, LRP7, LR3, OPPG, |
| 601813 (3) | VBCH2 |
| Exudative vitreoretinopathy, X-linked, | NDP, ND |
| 305390 (3) | |
| Eye anomalies, multiplex (3) | PAX6, AN2, MGDA |
| Ezetimibe, nonresponse to (3) | NPC1L1 |
| Fabry disease (3) | GLA |
| Facioscapulohumeral muscular dystrophy- | FSHMD1A, FSHD1A |
| 1A (3) | |
| Factor H and factor H-like 1 (3) | HF1, CFH, HUS |
| Factor V and factor VIII, combined | MCFD2 |
| deficiency of, 227300 (3) | |
| Factor VII deficiency (3) | F7 |
| Factor X deficiency (3) | F10 |
| Factor XI deficiency, autosomal dominant | F11 |
| (3) | |
| Factor XI deficiency, autosomal recessive | F11 |
| (3) | |
| Factor XII deficiency (3) | F12, HAF |
| Factor XIIIA deficiency (3) | F13A1, F13A |
| Factor XIIIB deficiency (3) | F13B |
| Familial Mediterranean fever, 249100 (3) | MEFV, MEF, FMF |
| Fanconi anemia, complementation group A, | FANCA, FACA, FA1, FA, FAA |
| 227650 (3) | |
| Fanconi anemia, complementation group B, | FAAP95, FAAP90, FLJ34064, FANCB |
| 300514 (3) | |
| Fanconi anemia, complementation group C | FANCC, FACC |
| (3) | |
| Fanconi anemia, complementation group | BRCA2, FANCD1 |
| D1, 605724 (3) | |
| Fanconi anemia, complementation group D2 | FANCD2, FANCD, FACD, FAD |
| (3) | |
| Fanconi anemia, complementation group E | FANCE, FACE |
| (3) | |
| Fanconi anemia, complementation group F | FANCF |
| (3) | |
| Fanconi anemia, complementation group G | XRCC9, FANCG |
| (3) | |
| Fanconi anemia, complementation group J, | BRIP1, BACH1, FANCJ |
| 609054 (3) | |
| Fanconi anemia, complementation group L | PHF9, FANCL |
| (3) | |
| Fanconi anemia, complementation group M | FANCM, KIAA1596 |
| (3) | |
| Fanconi-Bickel syndrome, 227810 (3) | SLC2A2, GLUT2 |
| Farber lipogranulomatosis (3) | ASAH, AC |
| Fatty liver, acute, of pregnancy (3) | HADHA, MTPA |
| Favism (3) | G6PD, G6PD1 |
| Fechtner syndrome, 153640 (3) | MYH9, MHA, FTNS, DFNA17 |
| Feingold syndrome, 164280 (3) | MYCN, NMYC, ODED, MODED |
| Fertile eunuch syndrome, 228300 (3) | GNRHR, LHRHR |
| Fibrocalculous pancreatic diabetes, | SPINK1, PSTI, PCTT, TATI |
| susceptibility to (3) | |
| Fibromatosis, gingival, 135300 (3) | SOS1, GINGF, GF1, HGF |
| Fibromatosis, juvenile hyaline, 228600 (3) | ANTXR2, CMG2, JHF, ISH |
| Fibrosis of extraocular muscles, congenital, | KIF21A, KIAA1708, FEOM1, CFEOM1 |
| 1, 135700 (3) | |
| Fibrosis of extraocular muscles, congenital, | PHOX2A, ARIX, CFEOM2 |
| 2, 602078 (3) | |
| Fibular hypoplasia and complex | GDF5, CDMP1 |
| brachydactyly, 228900 (3) | |
| Fish-eye disease, 136120 (3) | LCAT |
| Fish-odor syndrome, 602079 (3) | FMO3 |
| Fitzgerald factor deficiency (3) | KNG |
| Fluorouracil toxicity, sensitivity to (3) | DPYD, DPD |
| Focal cortical dysplasia, Taylor balloon cell | TSC1, LAM |
| type, 607341 (3) | |
| Follicle-stimulating hormone deficiency, | FSHB |
| isolated, 229070 (3) | |
| Forebrain defects (3) | TDGF1 |
| Foveal hypoplasia, isolated, 136520 (3) | PAX6, AN2, MGDA |
| Foveomacular dystrophy, adult-onset, with | RDS, RP7, PRPH2, PRPH, AVMD, |
| choroidal neovascularization, 608161 (3) | AOFMD |
| Fragile X syndrome (3) | FMR1, FRAXA |
| Fraser syndrome, 219000 (3) | FRAS1 |
| Fraser syndrome, 219000 (3) | FREM2 |
| Frasier syndrome, 136680 (3) | WT1 |
| Friedreich ataxia, 229300 (3) | FRDA, FARR |
| Friedreich ataxia with retained reflexes, | FRDA, FARR |
| 229300 (3) | |
| Frontometaphyseal dysplasia, 304120 (3) | FLNA, FLN1, ABPX, NHBP, OPD1, |
| | OPD2, FMD, MNS |
| Fructose-bisphosphatase deficiency (3) | FBP1 |
| Fructose intolerance (3) | ALDOB |
| Fructosuria (3) | KHK |
| Fuchs endothelial corneal dystrophy, | COL8A2, FECD, PPCD2 |
| 136800 (3) | |
| Fucosidosis (3) | FUCA1 |
| Fucosyltransferase-6 deficiency (3) | FUT6 |
| Fumarase deficiency, 606812 (3) | FH |
| Fundus albipunctatus, 136880 (3) | RDH5 |
| Fundus albipunctatus, 136880 (3) | RLBP1 |
| Fundus flavimaculatus, 248200 (3) | ABCA4, ABCR, STGD1, FFM, RP19 |
| G6PD deficiency (3) | G6PD, G6PD1 |
| GABA-transaminase deficiency (3) | ABAT, GABAT |
| Galactokinase deficiency with cataracts, | GALK1 |
| 230200 (3) | |
| Galactose epimerase deficiency, 230350 (3) | GALE |
| Galactosemia, 230400 (3) | GALT |
| Galactosialidosis (3) | PPGB, GSL, NGBE, GLB2, CTSA |
| GAMT deficiency (3) | GAMT |
| Gardner syndrome (3) | APC, GS, FPC |
| Gastric cancer, 137215 (3) | APC, GS, FPC |
| Gastric cancer, 137215 (3) | IRF1, MAR |
| Gastric cancer, familial diffuse, 137215 (3) | CDH1, UVO |
| Gastric cancer risk after H. pylori infection, | IL1B |
| 137215 (3) | |
| Gastric cancer risk after H. pylori infection, | IL1RN |
| 137215 (3) | |
| Gastric cancer, somatic, 137215 (3) | CASP10, MCH4, ALPS2 |
| Gastric cancer, somatic, 137215 (3) | ERBB2, NGL, NEU, HER2 |
| Gastric cancer, somatic, 137215 (3) | FGFR2, BEK, CFD1, JWS |
| Gastric cancer, somatic, 137215 (3) | KLF6, COPEB, BCD1, ZF9 |
| Gastric cancer, somatic, 137215 (3) | MUTYH |
| Gastrointestinal stromal tumor, somatic, | KIT, PBT |
| 606764 (3) | |
| Gastrointestinal stromal tumor, somatic, | PDGFRA |
| 606764 (3) | |
| Gaucher disease, 230800 (3) | GBA |
| Gaucher disease, variant form (3) | PSAP, SAP1 |
| Gaucher disease with cardiovascular | GBA |
| calcification, 231005 (3) | |
| Gaze palsy, horizontal, with progressive | ROBO3, RBIG1, RIG1, HGPPS |
| scoliosis, 607313 (3) | |
| Generalized epilepsy and paroxysmal | KCNMA1, SLO |
| dyskinesia, 609446 (3) | |
| Generalized epilepsy with febrile seizures | SCN1B, GEFSP1 |
| plus, 604233 (3) | |
| Germ cell tumor (3) | BCL10 |
| Germ cell tumors, 273300 (3) | KIT, PBT |
| Gerstmann-Straussler disease, 137440 (3) | PRNP, PRIP |
| Giant axonal neuropathy-1, 256850 (3) | GAN, GAN1 |
| Giant-cell fibroblastoma (3) | PDGFB, SIS |
| Giant cell hepatitis, neonatal, 231100 (3) | CYP7B1 |
| Giant platelet disorder, isolated (3) | GP1BB |
| Gilbert syndrome, 143500 (3) | UGT1A1, UGT1, GNT1 |
| Gitelman syndrome, 263800 (3) | SLC12A3, NCCT, TSC |
| Glanzmann thrombasthenia, type A, 273800 | ITGA2B, GP2B, CD41B |
| (3) | |
| Glanzmann thrombasthenia, type B (3) | ITGB3, GP3A |
| Glaucoma 1A, primary open angle, juvenile- | MYOC, TIGR, GLC1A, JOAG, GPOA |
| onset, 137750 (3) | |
| Glaucoma 1A, primary open angle, | MYOC, TIGR, GLC1A, JOAG, GPOA |
| recessive (3) | |
| Glaucoma 1E, primary open angle, adult- | OPTN, GLC1E, FIP2, HYPL, NRP |
| onset, 137760 (3) | |
| Glaucoma 3A, primary congenital, 231300 | CYP1B1, GLC3A |
| (3) | |
| Glaucoma, early-onset, digenic (3) | CYP1B1, GLC3A |
| Glaucoma, early-onset, digenic (3) | MYOC, TIGR, GLC1A, JOAG, GPOA |
| Glaucoma, normal tension, susceptibility to, | OPA1, NTG, NPG |
| 606657 (3) | |
| Glaucoma, normal tension, susceptibility to, | OPTN, GLC1E, FIP2, HYPL, NRP |
| 606657 (3) | |
| Glaucoma, primary open angle, adult-onset, | CYP1B1, GLC3A |
| 137760 (3) | |
| Glaucoma, primary open angle, juvenile- | CYP1B1, GLC3A |
| onset, 137750 (3) | |
| Glioblastoma, early-onset, 137800 (3) | MSH2, COCA1, FCC1, HNPCC1 |
| Glioblastoma multiforme, somatic, 137800 | DMBT1 |
| (3) | |
| Glioblastoma, somatic, 137800 (3) | ERBB2, NGL, NEU, HER2 |
| Glioblastoma, somatic, 137800 (3) | LGI1, EPT, ETL1 |
| Glioblastoma, susceptibility to, 137800 (3) | PPARG, PPARG1, PPARG2 |
| Glomerulocystic kidney disease, | TCF2, HNF2 |
| hypoplastic, 137920 (3) | |
| Glomerulosclerosis, focal segmental, 1, | ACTN4, FSGS1, FSGS |
| 603278 (3) | |
| Glomerulosclerosis, focal segmental, 2, | TRPC6, TRP6, FSGS2 |
| 603965 (3) | |
| Glomerulosclerosis, focal segmental, 3, | CD2AP, CMS |
| 607832 (3) | |
| Glomuvenous malformations, 138000 (3) | GLML, GVM, VMGLOM |
| Glucocorticoid deficiency 2, 607398 (3) | MRAP, FALP, C21orf61 |
| Glucocorticoid deficiency, due to ACTH | MC2R |
| unresponsiveness, 202200 (3) | |
| Glucose/galactose malabsorption, 606824 | SLC5A1, SGLT1 |
| (3) | |
| Glucose transport defect, blood-brain | SLC2A1, GLUT1 |
| barrier, 606777 (3) | |
| Glucosidase I deficiency, 606056 (3) | GCS1 |
| Glutamate formiminotransferase deficiency, | FTCD |
| 229100 (3) | |
| Glutaricaciduria, type I, 231670 (3) | GCDH |
| Glutaricaciduria, type IIA, 231680 (3) | ETFA, GA2, MADD |
| Glutaricaciduria, type IIB, 231680 (3) | ETFB, MADD |
| Glutaricaciduria, type IIC, 231680 (3) | ETFDH, MADD |
| Glutathione synthetase deficiency, 266130 | GSS, GSHS |
| (3) | |
| Glycerol kinase deficiency, 307030 (3) | GK |
| Glycine encephalopathy, 605899 (3) | AMT, NKH, GCE |
| Glycine encephalopathy, 605899 (3) | GCSH, NKH |
| Glycine encephalopathy, 605899 (3) | GLDC, HYGN1, GCSP, GCE, NKH |
| Glycine N-methyltransferase deficiency, | GNMT |
| 606664 (3) | |
| Glycogenosis, hepatic, autosomal (3) | PHKG2 |
| Glycogenosis, X-linked hepatic, type I (3) | PHKA2, PHK |
| Glycogenosis, X-linked hepatic, type II (3) | PHKA2, PHK |
| Glycogen storage disease I (3) | G6PC, G6PT |
| Glycogen storage disease Ib, 232220 (3) | G6PT1 |
| Glycogen storage disease Ic, 232240 (3) | G6PT1 |
| Glycogen storage disease II, 232300 (3) | GAA |
| Glycogen storage disease IIb, 300257 (3) | LAMP2, LAMPB |
| Glycogen storage disease IIIa (3) | AGL, GDE |
| Glycogen storage disease IIIb (3) | AGL, GDE |
| Glycogen storage disease IV, 232500 (3) | GBE1 |
| Glycogen storage disease, type 0, 240600 | GYS2 |
| (3) | |
| Glycogen storage disease VI (3) | PYGL |
| Glycogen storage disease VII (3) | PFKM |
| GM1-gangliosidosis (3) | GLB1 |
| GM2-gangliosidosis, AB variant (3) | GM2A |
| GM2-gangliosidosis, several forms, 272800 | HEXA, TSD |
| (3) | |
| Gnthodiaphyseal dysplasia, 166260 (3) | TMEM16E, GDD1 |
| Goiter, congenital (3) | TPO, TPX |
| Goiter, nonendemic, simple (3) | TG, AITD3 |
| Goldberg-Shprintzen megacolon syndrome, | KIAA1279 |
| 609460 (3) | |
| Gonadal dysgenesis, 46XY, partial, with | DHH |
| minifascicular neuropathy, 607080 (3) | |
| Gonadal dysgenesis, XY type (3) | SRY, TDF |
| GRACILE syndrome, 603358 (3) | BCS1L, FLNMS, GRACILE |
| Graft-versus-host disease, protection | IL10, CSIF |
| against (3) | |
| Graves disease, susceptibility to, 275000 (3) | CTLA4 |
| Graves disease, susceptibility to, 3, 275000 | GC, DBP |
| (3) | |
| Greenberg dysplasia, 215140 (3) | LBR, PHA |
| Greig cephalopolysyndactyly syndrome, | GLI3, PAPA, PAPB, ACLS |
| 175700 (3) | |
| Griscelli syndrome, type 1, 214450 (3) | MYO5A, MYH12, GS1 |
| Griscelli syndrome, type 2, 607624 (3) | RAB27A, RAM, GS2 |
| Griscelli syndrome, type 3, 609227 (3) | MLPH |
| Growth hormone deficient dwarfism (3) | GHRHR |
| Growth hormone insensitivity with | STAT5B |
| immunodeficiency, 245590 (3) | |
| Growth retardation with deafness and | IGF1 |
| mental retardation due to IGF1 deficiency, | |
| 608747 (3) | |
| Guttmacher syndrome, 176305 (3) | HOXA13, HOX1J |
| Gyrate atrophy of choroid and retina with | OAT |
| ornithinemia, B6 responsive or unresponsive | |
| (3) | |
| Hailey-Hailey disease, 169600 (3) | ATP2C1, BCPM, HHD |
| Haim-Munk syndrome, 245010 (3) | CTSC, CPPI, PALS, PLS, HMS |
| Hand-foot-uterus syndrome, 140000 (3) | HOXA13, HOX1J |
| Harderoporphyrinuria (3) | CPO |
| HARP syndrome, 607236 (3) | PANK2, NBIA1, PKAN, HARP |
| Hartnup disorder, 234500 (3) | SLC6A19, HND |
| Hay-Wells syndrome, 106260 (3) | TP73L, TP63, KET, EEC3, SHFM4, |
| | LMS, RHS |
| HDL deficiency, familial, 604091 (3) | ABCA1, ABC1, HDLDT1, TGD |
| HDL response to hormone replacement, | ESR1, ESR |
| augmented (3) | |
| Hearing loss, low-frequency sensorineural, | WFS1, WFRS, WFS, DFNA6 |
| 600965 (3) | |
| Heart block, nonprogressive, 113900 (3) | SCN5A, LQT3, IVF, HB1, SSS1 |
| Heart block, progressive, type I, 113900 (3) | SCN5A, LQT3, IVF, HB1, SSS1 |
| Heinz body anemia (3) | HBA2 |
| Heinz body anemias, alpha-(3) | HBA1 |
| Heinz body anemias, beta-(3) | HBB |
| HELLP syndrome, maternal, of pregnancy | HADHA, MTPA |
| (3) | |
| Hemangioblastoma, cerebellar, somatic (3) | VHL |
| Hemangioma, capillary infantile, somatic, | FLT4, VEGFR3, PCL |
| 602089 (3) | |
| Hemangioma, capillary infantile, somatic, | KDR |
| 602089 (3) | |
| Hematopoiesis, cyclic, 162800 (3) | ELA2 |
| Hematuria, familial benign (3) | COL4A4 |
| Heme oxygenase-1 deficiency (3) | HMOX1 |
| Hemiplegic migraine, familial, 141500 (3) | CACNA1A, CACNL1A4, SCA6 |
| Hemochromatosis (3) | HFE, HLA-H, HFE1 |
| Hemochromatosis, juvenile, 602390 (3) | HAMP, LEAP1, HEPC, HFE2 |
| Hemochromatosis, juvenile, digenic, 602390 | HAMP, LEAP1, HEPC, HFE2 |
| (3) | |
| Hemochromatosis, type 2A, 602390 (3) | HJV, HFE2A |
| Hemochromatosis, type 3, 604250 (3) | TFR2, HFE3 |
| Hemochromatosis, type 4, 606069 (3) | SLC40A1, SLC11A3, FPN1, IREG1, |
| | HFE4 |
| Hemoglobin H disease (3) | HBA2 |
| Hemolytic anemia due to adenylate kinase | AK1 |
| deficiency (3) | |
| Hemolytic anemia due to band 3 defect | SLC4A1, AE1, EPB3 |
| defect (3) | |
| Hemolytic anemia due to | BPGM |
| bisphosphoglycerate mutase deficiency (3) | |
| Hemolytic anemia due to G6PD deficiency | G6PD, G6PD1 |
| (3) | |
| Hemolytic anemia due to gamma- | GCLC, GLCLC |
| glutamylcysteine synthetase deficiency, | |
| 230450 (3) | |
| Hemolytic anemia due to glucosephosphate | GPI |
| isomerase deficiency (3) | |
| Hemolytic anemia due to glutathione | GSS, GSHS |
| synthetase deficiency, 231900 (3) | |
| Hemolytic anemia due to hexokinase | HK1 |
| deficiency (3) | |
| Hemolytic anemia due to PGK deficiency (3) | PGK1, PGKA |
| Hemolytic anemia due to triosephosphate | TPI1 |
| isomerase deficiency (3) | |
| Hemolytic-uremic syndrome, 235400 (3) | HF1, CFH, HUS |
| Hemophagocytic lymphohistiocytosis, | PRF1, HPLH2 |
| familial, 2, 603553 (3) | |
| Hemophagocytic lymphohistiocytosis, | UNC13D, MUNC13-4, HPLH3, HLH3, |
| familial, 3, 608898 (3) | FHL3 |
| Hemophilia A (3) | F8, F8C, HEMA |
| Hemophilia B (3) | F9, HEMB |
| Hemorrhagic diathesis due to | PI, AAT |
| \\grave{ }antithrombin\’ Pittsburgh (3) | |
| Hemorrhagic diathesis due to factor V | F5 |
| deficiency (3) | |
| Hemosiderosis, systemic, due to | CP |
| aceruloplasminemia, 604290 (3) | |
| Hepatic adenoma, 142330 (3) | TCF1, HNF1A, MODY3 |
| Hepatic failure, early onset, and neurologic | SCOD1, SCO1 |
| disorder (3) | |
| Hepatic lipase deficiency (3) | LIPC |
| Hepatoblastoma (3) | CTNNB1 |
| Hepatocellular cancer, 114550 (3) | PDGFRL, PDGRL, PRLTS |
| Hepatocellular carcinoma, 114550 (3) | AXIN1, AXIN |
| Hepatocellular carcinoma, 114550 (3) | CTNNB1 |
| Hepatocellular carcinoma, 114550 (3) | TP53, P53, LFS1 |
| Hepatocellular carcinoma (3) | IGF2R, MPRI |
| Hepatocellular carcinoma, childhood type, | MET |
| 114550 (3) | |
| Hepatocellular carcinoma, somatic, 114550 | CASP8, MCH5 |
| (3) | |
| Hereditary hemorrhagic telangiectasia-1, | ENG, END, HHT1, ORW |
| 187300 (3) | |
| Hereditary hemorrhagic telangiectasia-2, | ACVRL1, ACVRLK1, ALK1, HHT2 |
| 600376 (3) | |
| Hereditary persistence of alpha-fetoprotein | AFP, HPAFP |
| (3) | |
| Hermansky-Pudlak syndrome, 203300 (3) | HPS1 |
| Hermansky-Pudlak syndrome, 203300 (3) | HPS3 |
| Hermansky-Pudlak syndrome, 203300 (3) | HPS4 |
| Hermansky-pudlak syndrome, 203300 (3) | HPS5, RU2, KIAA1017 |
| Hermansky-Pudlak syndrome, 203300 (3) | HPS6, RU |
| Hermansky-Pudlak syndrome, 608233 (3) | AP3B1, ADTB3A, HPS2 |
| Hermansky-Pudlak syndrome 7, 203300 (3) | DTNBP1, HPS7 |
| Heterotaxy, visceral, 605376 (3) | CFC1, CRYPTIC, HTX2 |
| Heterotaxy, X-linked visceral, 306955 (3) | ZIC3, HTX1, HTX |
| Heterotopia, periventricular, 300049 (3) | FLNA, FLN1, ABPX, NHBP, OPD1, |
| | OPD2, FMD, MNS |
| Heterotopia, periventricular, ED variant, | FLNA, FLN1, ABPX, NHBP, OPD1, |
| 300537 (3) | OPD2, FMD, MNS |
| Heterotopia, periventricular nodular, with | FLNA, FLN1, ABPX, NHBP, OPD1, |
| frontometaphyseal dysplasia, 300049 (3) | OPD2, FMD, MNS |
| Hex A pseudodeficiency, 272800 (3) | HEXA, TSD |
| High-molecular-weight kininogen deficiency | KNG |
| (3) | |
| Hirschsprung disease, 142623 (3) | EDN3 |
| Hirschsprung disease, 142623 (3) | GDNF |
| Hirschsprung disease, 142623 (3) | NRTN, NTN |
| Hirschsprung disease, 142623 (3) | RET, MEN2A |
| Hirschsprung disease-2, 600155 (3) | EDNRB, HSCR2, ABCDS |
| Hirschsprung disease, cardiac defects, and | ECE1 |
| autonomic dysfunction (3) | |
| Hirschsprung disease, short-segment, | PMX2B, NBPHOX, PHOX2B |
| 142623 (3) | |
| Histidinemia, 235800 (3) | HAL, HSTD |
| Histiocytoma (3) | TP53, P53, LFS1 |
| HIV-1 disease, delayed progression of (3) | CCL5, SCYA5, D17S136E, TCP228 |
| HIV-1 disease, rapid progression of (3) | CCL5, SCYA5, D17S136E, TCP228 |
| HIV-1, susceptibility to (3) | IL10, CSIF |
| HIV infection, susceptibility/resistance to (3) | CMKBR2, CCR2 |
| HIV infection, susceptibility/resistance to (3) | CMKBR5, CCCKR5 |
| HMG-CoA lyase deficiency (3) | HMGCL |
| HMG-CoA synthase-2 deficiency, 605911 | HMGCS2 |
| (3) | |
| Holocarboxylase synthetase deficiency, | HLCS, HCS |
| 253270 (3) | |
| Holoprosencephaly-2, 157170 (3) | SIX3, HPE2 |
| Holoprosencephaly-3, 142945 (3) | SHH, HPE3, HLP3, SMMCI |
| Holoprosencephaly-4, 142946 (3) | TGIF, HPE4 |
| Holoprosencephaly-5, 609637 (3) | ZIC2, HPE5 |
| Holoprosencephaly-7 (3) | PTCH, NBCCS, BCNS, HPE7 |
| Holt-Oram syndrome, 142900 (3) | TBX5 |
| Homocysteine, total plasma, elevated (3) | CTH |
| Homocystinuria, B6-responsive and | CBS |
| nonresponsive types (3) | |
| Homocystinuria due to MTHFR deficiency, | MTHFR |
| 236250 (3) | |
| Homocystinuria-megaloblastic anemia, cbl E | MTRR |
| type, 236270 (3) | |
| Homozygous 2p16 deletion syndrome, | SLC3A1, ATR1, D2H, NBAT |
| 606407 (3) | |
| Hoyeraal-Hreidarsson syndrome, 300240 | DKC1, DKC |
| (3) | |
| HPFH, deletion type (3) | HBB |
| HPFH, nondeletion type A (3) | HBG1 |
| HPFH, nondeletion type G (3) | HBG2 |
| HPRT-related gout, 300323 (3) | HPRT1, HPRT |
| H. pylori infection, susceptibility to, 600263 | IFNGR1 |
| (3) | |
| Huntington disease (3) | HD, IT15 |
| Huntington disease-like 1, 603218 (3) | PRNP, PRIP |
| Huntington disease-like 2, 606438 (3) | JPH3, JP3, HDL2 |
| Huntington disease-like-4, 607136 (3) | TBP, SCA17 |
| Hyalinosis, infantile systemic, 236490 (3) | ANTXR2, CMG2, JHF, ISH |
| Hydrocephalus due to aqueductal stenosis, | L1CAM, CAML1, HSAS1 |
| 307000 (3) | |
| Hydrocephalus with congenital idiopathic | L1CAM, CAML1, HSAS1 |
| intestinal pseudoobstruction, 307000 (3) | |
| Hydrocephalus with Hirschsprung disease | L1CAM, CAML1, HSAS1 |
| and cleft palate, 142623 (3) | |
| Hyperalphalipoproteinemia, 143470 (3) | CETP |
| Hyperammonemia with hypoornithinemia, | PYCS, GSAS |
| hypocitrullinemia, hypoargininemia, and | |
| hypoprolinemia (3) | |
| Hyperandrogenism, nonclassic type, due to | CYP21A2, CYP21, CA21H |
| 21-hydroxylase deficiency (3) | |
| Hyperapobetalipoproteinemia, susceptibility | PPARA, PPAR |
| to (3) | |
| Hyperbilirubinemia, familial transcient | UGT1A1, UGT1, GNT1 |
| neonatal, 237900 (3) | |
| Hypercalciuria, absorptive, susceptibility to, | SAC, HCA2 |
| 143870 (3) | |
| Hypercholanemia, familial, 607748 (3) | BAAT |
| Hypercholanemia, familial, 607748 (3) | EPHX1 |
| Hypercholanemia, familial, 607748 (3) | TJP2, ZO2 |
| Hypercholesterolemia, due to ligand- | APOB, FLDB |
| defective apo B, 144010 (3) | |
| Hypercholesterolemia, familial, 143890 (3) | LDLR, FHC, FH |
| Hypercholesterolemia, familial, 3, 603776 | PCSK9, NARC1, HCHOLA3, FH3 |
| (3) | |
| Hypercholesterolemia, familial, autosomal | ARH, FHCB2, FHCB1 |
| recessive, 603813 (3) | |
| Hypercholesterolemia, familial, due to LDLR | EPHX2 |
| defect, modifier of, 143890 (3) | |
| Hypercholesterolemia, familial, modification | APOA2 |
| of, 143890 (3) | |
| Hypercholesterolemia, susceptibility to, | GSBS |
| 143890 (3) | |
| Hypercholesterolemia, susceptibility to, | ITIH4, PK120, ITIHL1 |
| 143890 (3) | |
| Hyperekplexia and spastic paraparesis (3) | GLRA1, STHE |
| Hyperekplexia, autosomal recessive, | GLRB |
| 149400 (3) | |
| Hypereosinophilic syndrome, idiopathic, | PDGFRA |
| resistant to imatinib, 607685 (3) | |
| Hyperferritinemia-cataract syndrome, | FTL |
| 600886 (3) | |
| Hyper-IgD syndrome, 260920 (3) | MVK, MVLK |
| Hyperinsulinism, familial, 602485 (3) | GCK |
| Hyperinsulinism-hyperammonemia | GLUD1 |
| syndrome, 606762 (3) | |
| Hyperkalemic periodic paralysis, 170500 (3) | SCN4A, HYPP, NAC1A |
| Hyperkeratotic cutaneous capillary-venous | CCM1, CAM, KRIT1 |
| malformations associated with cerebral | |
| capillary malformations, 116860 (3) | |
| Hyperlipidemia, familial combined, | USF1, HYPLIP1 |
| susceptibility to, 602491 (3) | |
| Hyperlipoproteinemia, type Ib, 207750 (3) | APOC2 |
| Hyperlipoproteinemia, type III (3) | APOE, AD2 |
| Hyperlysinemia, 238700 (3) | AASS |
| Hypermethioninemia, persistent, autosomal | MAT1A, MATA1, SAMS1 |
| dominant, due to methionine | |
| adenosyltransferase I/III deficiency (3) | |
| Hypermethioninemia with deficiency of S- | AHCY, SAHH |
| adenosylhomocysteine hydrolase (3) | |
| Hyperornithinemia-hyperammonemia- | SLC25A15, ORNT1, HHH |
| homocitrullinemia syndrome, 238970 (3) | |
| Hyperostosis, endosteal, 144750 (3) | LRP5, BMND1, LRP7, LR3, OPPG, |
| | VBCH2 |
| Hyperoxaluria, primary, type 1, 259900 (3) | AGXT, SPAT |
| Hyperoxaluria, primary, type II, 260000 (3) | GRHPR, GLXR |
| Hyperparathyroidism, AD, 145000 (3) | MEN1 |
| Hyperparathyroidism, familial primary, | HRPT2, C1orf28 |
| 145000 (3) | |
| Hyperparathyroidism-jaw tumor syndrome, | HRPT2, C1orf28 |
| 145001 (3) | |
| Hyperparathyroidism, neonatal, 239200 (3) | CASR, HHC1, PCAR1, FIH |
| Hyperphenylalaninemia due to pterin-4a- | PCBD, DCOH |
| carbinolamine dehydratase deficiency, | |
| 264070 (3) | |
| Hyperphenylalaninemia, mild (3) | PAH, PKU1 |
| Hyperproinsulinemia, familial (3) | INS |
| Hyperprolinemia, type I, 239500 (3) | PRODH, PRODH2, SCZD4 |
| Hyperprolinemia, type II, 239510 (3) | ALDH4A1, ALDH4, P5CDH |
| Hyperproreninemia (3) | REN |
| Hyperprothrombinemia (3) | F2 |
| Hypertension, diastolic, resistance to, | KCNMB1 |
| 608622 (3) | |
| Hypertension, early-onset, autosomal | NR3C2, MLR, MCR |
| dominant, with exacerbation in pregnancy, | |
| 605115 (3) | |
| Hypertension, essential, 145500 (3) | AGTR1, AGTR1A, AT2R1 |
| Hypertension, essential, 145500 (3) | PTGIS, CYP8A1, PGIS, CYP8 |
| Hypertension, essential, salt-sensitive, | ADD1 |
| 145500 (3) | |
| Hypertension, essential, susceptibility to, | AGT, SERPINA8 |
| 145500 (3) | |
| Hypertension, essential, susceptibility to, | ECE1 |
| 145500 (3) | |
| Hypertension, essential, susceptibility to, | GNB3 |
| 145500 (3) | |
| Hypertension, insulin resistance-related, | RETN, RSTN, FIZZ3 |
| susceptibility to, 125853 (3) | |
| Hypertension, mild low-renin (3) | HSD11B2, HSD11K |
| Hypertension, pregnancy-induced, 189800 | NOS3 |
| (3) | |
| Hypertension, salt-sensitive essential, | CYP3A5, P450PCN3 |
| susceptibility to, 145500 (3) | |
| Hypertension, susceptibility to, 145500 (3) | NOS3 |
| Hyperthroidism, congenital (3) | TSHR |
| Hyperthyroidism, congenital (3) | TPO, TPX |
| Hypertriglyceridemia, one form (3) | APOA1 |
| Hypertriglyceridemia, susceptibility to, | APOA5 |
| 145750 (3) | |
| Hypertriglyceridemia, susceptibility to, | LIPI, LPDL, PRED5 |
| 145750 (3) | |
| Hypertriglyceridemia, susceptibility to, | RP1, ORP1 |
| 145750 (3) | |
| Hypertrypsinemia, neonatal (3) | CFTR, ABCC7, CF, MRP7 |
| Hyperuricemic nephropathy, familial | UMOD, HNFJ, FJHN, MCKD2, |
| juvenile, 162000 (3) | ADMCKD2 |
| Hypoaldosteronism, congenital, due to CMO | CYP11B2 |
| I deficiency, 203400 (3) | |
| Hypoaldosteronism, congenital, due to CMO | CYP11B2 |
| II deficiency (3) | |
| Hypoalphalipoproteinemia (3) | APOA1 |
| Hypobetalipoproteinemia (3) | APOB, FLDB |
| Hypocalcemia, autosomal dominant, | CASR, HHC1, PCAR1, FIH |
| 146200 (3) | |
| Hypocalcemia, autosomal dominant, with | CASR, HHC1, PCAR1, FIH |
| Bartter syndrome (3) | |
| Hypocalciuric hypercalcemia, type I, 145980 | CASR, HHC1, PCAR1, FIH |
| (3) | |
| Hypoceruloplasminemia, hereditary, 604290 | CP |
| (3) | |
| Hypochondroplasia, 146000 (3) | FGFR3, ACH |
| Hypochromic microcytic anemia (3) | HBA2 |
| Hypodontia, 106600 (3) | PAX9 |
| Hypodontia, autosomal dominant, 106600 | MSX1, HOX7, HYD1, OFC5 |
| (3) | |
| Hypodontia with orofacial cleft, 106600 (3) | MSX1, HOX7, HYD1, OFC5 |
| Hypofibrinogenemia, gamma type (3) | FGG |
| Hypoglobulinemia and absent B cells (3) | BLNK, SLP65 |
| Hypoglycemia of infancy, leucine-sensitive, | ABCC8, SUR, PHHI, SUR1 |
| 240800 (3) | |
| Hypoglycemia of infancy, persistent | ABCC8, SUR, PHHI, SUR1 |
| hyperinsulinemic, 256450 (3) | |
| Hypogonadism, hypergonadotropic (3) | LHB |
| Hypogonadotropic hypogonadism, 146110 | GPR54 |
| (3) | |
| Hypogonadotropic hypogonadism, 146110 | NELF |
| (3) | |
| Hypogonadotropic hypogonadism (3) | GNRHR, LHRHR |
| Hypogonadotropic hypogonadism (3) | LHCGR |
| Hypohaptoglobinemia (3) | HP |
| Hypokalemic periodic paralysis, 170400 (3) | CACNA1S, CACNL1A3, CCHL1A3 |
| Hypokalemic periodic paralysis, 170400 (3) | KCNE3, HOKPP |
| Hypokalemic periodic paralysis, 170400 (3) | SCN4A, HYPP, NAC1A |
| Hypolactasia, adult type, 223100 (3) | LCT, LAC, LPH |
| Hypolactasia, adult type, 223100 (3) | MCM6 |
| Hypomagnesemia-2, renal, 154020 (3) | FXYD2, ATP1G1, HOMG2 |
| Hypomagnesemia, primary, 248250 (3) | CLDN16, PCLN1 |
| Hypomagnesemia with secondary | TRPM6, CHAK2 |
| hypocalcemia, 602014 (3) | |
| Hypoparathyroidism, autosomal dominant(3) | PTH |
| Hypoparathyroidism, autosomal recessive | PTH |
| (3) | |
| Hypoparathyroidism, familial isolated, | GCMB |
| 146200 (3) | |
| Hypoparathyroidism-retardation- | TBCE, KCS, KCS1, HRD |
| dysmorphism syndrome, 241410 (3) | |
| Hypoparathyroidism, sensorineural | GATA3, HDR |
| deafness, and renal dysplasia, 146255 (3) | |
| Hypophosphatasia, childhood, 241510 (3) | ALPL, HOPS, TNSALP |
| Hypophosphatasia, infantile, 241500 (3) | ALPL, HOPS, TNSALP |
| Hypophosphatemia, type III (3) | CLCN5, CLCK2, NPHL2, DENTS |
| Hypophosphatemia, X-linked, 307800 (3) | PHEX, HYP, HPDR1 |
| Hypophosphatemic rickets, autosomal | FGF23, ADHR, HPDR2, PHPTC |
| dominant, 193100 (3) | |
| Hypoplastic enamel pitting, localized, | ENAM |
| 608563 (3) | |
| Hypoplastic left heart syndrome, 241550 (3) | GJA1, CX43, ODDD, SDTY3, ODOD |
| Hypoprothrombinemia (3) | F2 |
| Hypothyroidism, autoimmune, 140300 (3) | CTLA4 |
| Hypothyroidism, congenital, 274400 (3) | SLC5A5, NIS |
| Hypothyroidism, congenital, due to DUOX2 | DUOX2, THOX2 |
| deficiency, 607200 (3) | |
| Hypothyroidism, congenital, due to thyroid | PAX8 |
| dysgenesis or hypoplasia, 218700 (3) | |
| Hypothyroidism, congenital, due to TSH | TSHR |
| resistance, 275200 (3) | |
| Hypothyroidism, hereditary congenital (3) | TG, AITD3 |
| Hypothyroidism, nongoitrous (3) | TSHB |
| Hypothyroidism, subclinical (3) | TSHR |
| Hypotrichosis, congential, with juvenile | CDH3, CDHP, PCAD, HJMD |
| macular dystrophy, 601553 (3) | |
| Hypotrichosis, localized, autosomal | DSG4, LAH |
| recessive, 607903 (3) | |
| Hypotrichosis-lymphedema-telangiectasia | SOX18, HLTS |
| syndrome, 607823 (3) | |
| Hypotrichosis simplex of scalp, 146520 (3) | CDSN, HTSS |
| Hypouricemia, renal, 220150 (3) | SLC22A12, OAT4L, URAT1 |
| Hystrix-like ichthyosis with deafness, | GJB2, CX26, DFNB1, PPK, DFNA3, |
| 602540 (3) | KID, HID |
| Ichthyosiform erythroderma, congenital, | TGM1, ICR2, LI1 |
| 242100 (3) | |
| Ichthyosiform erythroderma, congenital, | ALOX12B |
| nonbullous, 1, 242100 (3) | |
| Ichthyosiform erythroderma, congenital, | ALOXE3 |
| nonbullous, 1, 242100 (3) | |
| Ichthyosis bullosa of Siemens, 146800 (3) | KRT2A, KRT2E |
| Ichthyosis, congenital, autosomal recessive | ICHYN |
| (3) | |
| Ichthyosis, cyclic, with epidermolytic | KRT10 |
| hyperkeratosis, 607602 (3) | |
| Ichthyosis, harlequin, 242500 (3) | ABCA12, ICR2B, LI2 |
| Ichthyosis histrix, Curth-Macklin type, | KRT1 |
| 146590 (3) | |
| Ichthyosis, lamellar 2, 601277 (3) | ABCA12, ICR2B, LI2 |
| Ichthyosis, lamellar, autosomal recessive, | TGM1, ICR2, LI1 |
| 242300 (3) | |
| Ichthyosis, X-linked (3) | STS, ARSC1, ARSC, SSDD |
| ICOS deficiency, 607594 (3) | ICOS, AILIM |
| IgE levels QTL, 147050 (3) | PHF11, NYREN34 |
| IgG2 deficiency, selective (3) | IGHG2 |
| IgG receptor I, phagocytic, familial | FCGR1A, IGFR1, CD64 |
| deficiency of (3) | |
| Immunodeficiency-centromeric instability- | DNMT3B, ICF |
| facial anomalies syndrome, 242860 (3) | |
| Immunodeficiency due to defect in CD3- | CD3E |
| epsilon (3) | |
| Immunodeficiency due to defect in CD3- | CD3G |
| gamma (3) | |
| Immunodeficiency with hyper-IgM, type 2, | AICDA, AID, HIGM2 |
| 605258 (3) | |
| Immunodeficiency with hyper-IgM, type 3, | TNFRSF5, CD40 |
| 606843 (3) | |
| Immunodeficiency with hyper IgM, type 4, | UNG, DGU, HIGM4 |
| 608106 (3) | |
| Immunodeficiency, X-linked, with hyper-IgM, | TNFSF5, CD40LG, HIGM1, IGM |
| 308230 (3) | |
| Immunodysregulation, polyendocrinopathy, | FOXP3, IPEX, AIID, XPID, PIDX |
| and enteropathy, X-linked, 304790 (3) | |
| Immunoglobulin A deficiency, 609529 (3) | TNFRSF14B, TACI |
| Inclusion body myopathy-3, 605637 (3) | MYH2 |
| Inclusion body myopathy, autosomal | GNE, GLCNE, IBM2, DMRV, NM |
| recessive, 600737 (3) | |
| Inclusion body myopathy with early-onset | VCP, IBMPFD |
| Paget disease and frontotemporal dementia, | |
| 167320 (3) | |
| Incontinentia pigmenti, type II, 308300 (3) | IKBKG, NEMO, FIP3, IP2 |
| Infantile spasm syndrome, 308350 (3) | ARX, ISSX, PRTS, MRXS1, MRX36, |
| | MRX54 |
| Infundibular hypoplasia and hypopituitarism | SOX3, MRGH |
| (3) | |
| Inosine triphosphatase deficiency (3) | ITPA |
| Insensitivity to pain, congenital, with | NTRK1, TRKA, MTC |
| anhidrosis, 256800 (3) | |
| Insomnia (3) ( ) | GABRB3 |
| Insomnia, fatal familial, 600072 (3) | PRNP, PRIP |
| Insulin resistance, severe, digenic, 604367 | PPARG, PPARG1, PPARG2 |
| (3) | |
| Insulin resistance, severe, digenic, 604367 | PPP1R3A, PPP1R3 |
| (3) | |
| Insulin resistance, susceptibility to (3) | PTPN1, PTP1B |
| Interleukin-2 receptor, alpha chain, | IL2RA, IL2R |
| deficiency of (3) | |
| Intervertebral disc disease, susceptibility to, | COL9A2, EDM2 |
| 603932 (3) | |
| Intervertebral disc disease, susceptibility to, | COL9A3, EDM3, IDD |
| 603932 (3) | |
| Intrauterine and postnatal growth retardation | IGF1R |
| (3) | |
| Intrauterine and postnatal growth retardation | IGF2 |
| (3) | |
| Intrinsic factor deficiency, 261000 (3) | GIF, IF |
| IRAK4 deficiency, 607676 (3) | IRAK4, REN64 |
| Iridogoniodysgenesis, 601631 (3) | FOXC1, FKHL7, FREAC3 |
| Iridogoniodysgenesis syndrome-2, 137600 | PITX2, IDG2, RIEG1, RGS, IGDS2 |
| (3) | |
| Iris hypoplasia and glaucoma (3) | FOXC1, FKHL7, FREAC3 |
| Iron deficiency anemia, susceptibility to (3) | TF |
| Iron overload, autosomal dominant (3) | FTH1, FTHL6 |
| Isolated growth hormone deficiency, IIIig | GH1, GHN |
| type with absent GH and Kowarski type with | |
| bioinactive GH (3) | |
| Isovaleric acidemia, 243500 (3) | IVD |
| Jackson-Weiss syndrome, 123150 (3) | FGFR1, FLT2, KAL2 |
| Jackson-Weiss syndrome, 123150 (3) | FGFR2, BEK, CFD1, JWS |
| Jensen syndrome, 311150 (3) | TIMM8A, DFN1, DDP, MTS, DDP1 |
| Jervell and Lange-Nielsen syndrome, | KCNE1, JLNS, LQT5 |
| 220400 (3) | |
| Jervell and Lange-Nielsen syndrome, | KCNQ1, KCNA9, LQT1, KVLQT1, |
| 220400 (3) | ATFB1 |
| Joubert syndrome, 213300 (3) | NPHP1, NPH1, SLSN1 |
| Joubert syndrome-3, 608629 (3) | AHI1 |
| Juberg-Marsidi syndrome, 309590 (3) | ATRX, XH2, XNP, MRXS3, SHS |
| Juvenile polyposis/hereditary hemorrhagic | MADH4, DPC4, SMAD4, JIP |
| telangiectasia syndrome, 175050 (3) | |
| Kallikrein, decreased urinary activity of (3) | KLK1, KLKR |
| Kallmann syndrome 2, 147950 (3) | FGFR1, FLT2, KAL2 |
| Kallmann syndrome (3) | KAL1, KMS, ADMLX |
| Kanzaki disease, 609242 (3) | NAGA |
| Kaposi sarcoma, susceptibility to, 148000 | IL6, IFNB2, BSF2 |
| (3) | |
| Kappa light chain deficiency (3) | IGKC |
| Kartagener syndrome, 244400 (3) | DNAH11, DNAHC11 |
| Kartagener syndrome, 244400 (3) | DNAH5, HL1, PCD, CILD3 |
| Kartagener syndrome, 244400 (3) | DNAI1, CILD1, ICS, PCD |
| Kenny-Caffey syndrome-1, 244460 (3) | TBCE, KCS, KCS1, HRD |
| Keratitis, 148190 (3) | PAX6, AN2, MGDA |
| Keratitis-ichthyosis-deafness syndrome, | GJB2, CX26, DFNB1, PPK, DFNA3, |
| 148210 (3) | KID, HID |
| Keratoconus, 148300 (3) | VSX1, RINX, PPCD, PPD, KTCN |
| Keratoderma, palmoplantar, with deafness, | GJB2, CX26, DFNB1, PPK, DFNA3, |
| 148350 (3) | KID, HID |
| Keratosis follicularis spinulosa decalvans, | SAT, SSAT, KFSD |
| 308800 (3) | |
| Keratosis palmoplantaria striata, 148700 (3) | KRT1 |
| Keratosis palmoplantaris striata I, 148700 | DSG1 |
| (3) | |
| Keratosis palmoplantaris striata II (3) | DSP, KPPS2, PPKS2 |
| Keratosis palmoplantaris striata III, 607654 | KRT1 |
| (3) | |
| Ketoacidosis due to SCOT deficiency (3) | SCOT, OXCT |
| Keutel syndrome, 245150 (3) | MGP, NTI |
| Kindler syndrome, 173650 (3) | KIND1, URP1, C20orf42 |
| Kininogen deficiency (3) | KNG |
| Klippel-Trenaunay syndrome, 149000 (3) | VG5Q, HUS84971, FLJ10283 |
| Kniest dysplasia, 156550 (3) | COL2A1 |
| Knobloch syndrome, 267750 (3) | COL18A1, KNO |
| Krabbe disease, 245200 (3) | GALC |
| L-2-hydroxyglutaric aciduria, 236792 (3) | L2HGDH, C14orf160 |
| Lactate dehydrogenase-B deficiency (3) | LDHB |
| Lacticacidemia due to PDX1 deficiency, | PDX1 |
| 245349 (3) | |
| Langer mesomelic dysplasia, 249700 (3) | SHOX, GCFX, SS, PHOG |
| Langer mesomelic dysplasia, 249700 (3) | SHOXY |
| Laron dwarfism, 262500 (3) | GHR |
| Larson syndrome, 150250 (3) | FLNB, SCT, AOI |
| Laryngoonychocutaneous syndrome, | LAMA3, LOCS |
| 245660 (3) | |
| Lathosterolosis, 607330 (3) | SC5DL, ERG3 |
| LCHAD deficiency (3) | HADHA, MTPA |
| Lead poisoning, susceptibility to (3) | ALAD |
| Leanness, inherited (3) | AGRP, ART, AGRT |
| Leber congenital amaurosis, 204000 (3) | CRB1, RP12 |
| Leber congenital amaurosis, 204000 (3) | CRX, CORD2, CRD |
| Leber congenital amaurosis, 204000 (3) | RPGRIP1, LCA6, CORD9 |
| Leber congenital amaurosis-2, 204100 (3) | RPE65, RP20 |
| Leber congenital amaurosis, 604393 (3) | AIPL1, LCA4 |
| Leber congenital amaurosis, type I, 204000 | GUCY2D, GUC2D, LCA1, CORD6 |
| (3) | |
| Leber congenital amaurosis, type III, | RDH12, LCA3 |
| 604232 (3) | |
| Left-right axis malformations (3) | ACVR2B |
| Left-right axis malformations (3) | EBAF, TGFB4, LEFTY2, LEFTA, |
| | LEFTYA |
| Left ventricular noncompaction, familial | DTNA, D18S892E, DRP3, LVNC1 |
| isolated, 1, 604169 (3) | |
| Left ventricular noncompaction with | DTNA, D18S892E, DRP3, LVNC1 |
| congenital heart defects, 606617 (3) | |
| Legionaire disease, susceptibility to, 608556 | TLR5, TIL3 |
| (3) | |
| Leigh syndrome, 256000 (3) | BCS1L, FLNMS, GRACILE |
| Leigh syndrome, 256000 (3) | DLD, LAD, PHE3 |
| Leigh syndrome, 256000 (3) | NDUFS3 |
| Leigh syndrome, 256000 (3) | NDUFS4, AQDQ |
| Leigh syndrome, 256000 (3) | NDUFS7, PSST |
| Leigh syndrome, 256000 (3) | NDUFS8 |
| Leigh syndrome, 256000 (3) | NDUFV1, UQOR1 |
| Leigh syndrome, 256000 (3) | SDHA, SDH2, SDHF |
| Leigh syndrome, due to COX deficiency, | SURF1 |
| 256000 (3) | |
| Leigh syndrome due to cytochrome c | COX15 |
| oxidase deficiency, 256000 (3) | |
| Leigh syndrome, French-Canadian type, | LRPPRC, LRP130, LSFC |
| 220111 (3) | |
| Leigh syndrome, X-linked, 308930 (3) | PDHA1, PHE1A |
| Leiomyomatosis and renal cell cancer, | FH |
| 605839 (3) | |
| Leiomyomatosis, diffuse, with Alport | COL4A6 |
| syndrome, 308940 (3) | |
| Leopard syndrome, 151100 (3) | PTPN11, PTP2C, SHP2, NS1 |
| Leprechaunism, 246200 (3) | INSR |
| Leprosy, susceptibility to, 607572 (3) | PRKN, PARK2, PDJ |
| Leri-Weill dyschondrosteosis, 127300 (3) | SHOX, GCFX, SS, PHOG |
| Leri-Weill dyschondrosteosis, 127300 (3) | SHOXY |
| Lesch-Nyhan syndrome, 300322, (3) | HPRT1, HPRT |
| Leukemia-1, T-cell acute lymphocytic (3) | TAL1, TCL5, SCL |
| Leukemia-2, T-cell acute lymphoblastic (3) | TAL2 |
| Leukemia, acute lymphoblastic (3) | FLT3 |
| Leukemia, acute lymphoblastic (3) | NBS1, NBS |
| Leukemia, acute lymphoblastic (3) | ZNFN1A1, IK1, LYF1 |
| Leukemia, acute lymphoblastic, | HOXD4, HOX4B |
| susceptibility to (3) | |
| Leukemia, acute lymphocytic (3) | BCR, CML, PHL, ALL |
| Leukemia, acute myeloblastic (3) | ARNT |
| Leukemia, acute myelogenous (3) | KRAS2, RASK2 |
| Leukemia, acute myelogenous, 601626 (3) | GMPS |
| Leukemia, acute myeloid, 601626 (3) | AF10 |
| Leukemia, acute myeloid, 601626 (3) | ARHGEF12, LARG, KIAA0382 |
| Leukemia, acute myeloid, 601626 (3) | CALM, CLTH |
| Leukemia, acute myeloid, 601626 (3) | CEBPA, CEBP |
| Leukemia, acute myeloid, 601626 (3) | CHIC2, BTL |
| Leukemia, acute myeloid, 601626 (3) | FLT3 |
| Leukemia, acute myeloid, 601626 (3) | KIT, PBT |
| Leukemia, acute myeloid, 601626 (3) | LPP |
| Leukemia, acute myeloid, 601626 (3) | NPM1 |
| Leukemia, acute myeloid, 601626 (3) | NUP214, D9S46E, CAN, CAIN |
| Leukemia, acute myeloid, 601626 (3) | RUNX1, CBFA2, AML1 |
| Leukemia, acute myeloid, 601626 (3) | WHSC1L1, NSD3 |
| Leukemia, acute myeloid, reduced survival | FLT3 |
| in (3) | |
| Leukemia, acute myelomonocytic (3) | AF1Q |
| Leukemia, acute promyelocytic, NPM/RARA | NPM1 |
| type (3) | |
| Leukemia, acute promyelocytic, | NUMA1 |
| NUMA/RARA type (3) | |
| Leukemia, acute promyelocytic, | ZNF145, PLZF |
| PL2F/RARA type (3) | |
| Leukemia, acute promyelocytic, PML/RARA | PML, MYL |
| type (3) | |
| Leukemia, acute promyeloyctic, | STAT5B |
| STAT5B/RARA type (3) | |
| Leukemia, acute T-cell lymphoblastic (3) | AF10 |
| Leukemia, acute T-cell lymphoblastic (3) | CALM, CLTH |
| Leukemia, chronic lymphatic, susceptibility | ARL11, ARLTS1 |
| to, 151400 (3) | |
| Leukemia, chronic lymphatic, susceptibility | P2RX7, P2X7 |
| to, 151400 (3) | |
| Leukemia, chronic myeloid, 608232 (3) | BCR, CML, PHL, ALL |
| Leukemia, juvenile myelomonocytic, 607785 | GRAF |
| (3) | |
| Leukemia, juvenile myelomonocytic, 607785 | NF1, VRNF, WSS, NFNS |
| (3) | |
| Leukemia, juvenile myelomonocytic, 607785 | PTPN11, PTP2C, SHP2, NS1 |
| (3) | |
| Leukemia/lymphoma, B-cell, 2 (3) | BCL2 |
| Leukemia/lymphoma, chronic B-cell, 151400 | CCND1, PRAD1, BCL1 |
| (3) | |
| Leukemia/lymphoma, T-cell (3) | TCRA |
| Leukemia, megakaryoblastic, of Down | GATA1, GF1, ERYF1, NFE1 |
| syndrome, 190685 (3) | |
| Leukemia, megakaryoblastic, with or without | GATA1, GF1, ERYF1, NFE1 |
| Down syndrome, 190685 (3) | |
| Leukemia, Philadelphia chromosome- | ABL1 |
| positive, resistant to imatinib (3) | |
| Leukemia, post-chemotherapy, susceptibility | NQO1, DIA4, NMOR1 |
| to (3) | |
| Leukemia, T-cell acute lymphoblastic (3) | NUP214, D9S46E, CAN, CAIN |
| Leukocyte adhesion deficiency, 116920 (3) | ITGB2, CD18, LCAMB, LAD |
| Leukoencephalopathy with vanishing white | EIF2B1, EIF2BA |
| matter, 603896 (3) | |
| Leukoencephalopathy with vanishing white | EIF2B2 |
| matter, 603896 (3) | |
| Leukoencephalopathy with vanishing white | EIF2B3 |
| matter, 603896 (3) | |
| Leukoencephalopathy with vanishing white | EIF2B5, LVWM, CACH, CLE |
| matter, 603896 (3) | |
| Leukoencephaly with vanishing white | EIF2B4 |
| matter, 603896 (3) | |
| Leydig cell adenoma, with precocious | LHCGR |
| puberty (3) | |
| Lhermitte-Duclos syndrome (3) | PTEN, MMAC1 |
| Liddle syndrome, 177200 (3) | SCNN1B |
| Liddle syndrome, 177200 (3) | SCNN1G, PHA1 |
| Li Fraumeni syndrome, 151623 (3) | CDKN2A, MTS1, P16, MLM, CMM2 |
| Li-Fraumeni syndrome, 151623 (3) | TP53, P53, LFS1 |
| Li-Fraumeni syndrome, 609265 (3) | CHEK2, RAD53, CHK2, CDS1, LFS2 |
| LIG4 syndrome, 606593 (3) | LIG4 |
| Limb-mammary syndrome, 603543 (3) | TP73L, TP63, KET, EEC3, SHFM4, |
| | LMS, RHS |
| Lipodystrophy, congenital generalized, type | AGPAT2, LPAAB, BSCL, BSCL1 |
| 1, 608594 (3) | |
| Lipodystrophy, congenital generalized, type | BSCL2, SPG17 |
| 2, 269700 (3) | |
| Lipodystrophy, familial partial, 151660 (3) | LMNA, LMN1, EMD2, FPLD, CMD1A, |
| | HGPS, LGMD1B |
| Lipodystrophy, familial partial, 151660 (3) | PPARG, PPARG1, PPARG2 |
| Lipodystrophy, familial partial, with | PPARGC1A, PPARGC1 |
| decreased subcutaneous fat of face and | |
| neck (3) | |
| Lipoid adrenal hyperplasia, 201710 (3) | STAR |
| Lipoid congenital adrenal hyperplasia, | CYP11A, P450SCC |
| 201710 (3) | |
| Lipoid proteinosis, 247100 (3) | ECM1 |
| Lipoma (3) | HMGA2, HMGIC, BABL, LIPO |
| Lipoma (3) | LPP |
| Lipoma, sporadic (3) | MEN1 |
| Lipomatosis, mutiple, 151900 (3) | HMGA2, HMGIC, BABL, LIPO |
| Lipoprotein lipase deficiency (3) | LPL, LIPD |
| Lissencephaly-1, 607432 (3) | PAFAH1B1, LIS1 |
| Lissencephaly syndrome, Norman-Roberts | RELN, RL |
| type, 257320 (3) | |
| Lissencephaly, X-linked, 300067 (3) | DCX, DBCN, LISX |
| Lissencephaly, X-linked with ambiguous | ARX, ISSX, PRTS, MRXS1, MRX36, |
| genitalia, 300215 (3) | MRX54 |
| Listeria monocytogenes, susceptibility to (3) | CDH1, UVO |
| Loeys-Dietz syndrome, 609192 (3) | TGFBR1 |
| Loeys-Dietz syndrome, 609192 (3) | TGFBR2, HNPCC6 |
| Longevity, exceptional, 152430 (3) | CETP |
| Longevity, reduced, 152430 (3) | AKAP10 |
| Long QT syndrome-1, 192500 (3) | KCNQ1, KCNA9, LQT1, KVLQT1, |
| | ATFB1 |
| Long QT syndrome-2 (3) | KCNH2, LQT2, HERG |
| Long QT syndrome-3, 603830 (3) | SCN5A, LQT3, IVF, HB1, SSS1 |
| Long QT syndrome 4, 600919 (3) | ANK2, LQT4 |
| Long QT syndrome-5 (3) | KCNE1, JLNS, LQT5 |
| Long QT syndrome-6 (3) | KCNE2, MIRP1, LQT6 |
| Long QT syndrome-7, 170390 (3) | KCNJ2, HHIRK1, KIR2.1, IRK1, LQT7 |
| Lower motor neuron disease, progressive, | DCTN1 |
| without sensory symptoms, 607641 (3) | |
| Lowe syndrome, 309000 (3) | OCRL, LOCR, OCRL1, NPHL2 |
| Low renin hypertension, susceptibility to (3) | CYP11B2 |
| LPA deficiency, congenital (3) | LPA |
| Lumbar disc disease, susceptibility to, | CILP |
| 603932 (3) | |
| Lung cancer, 211980 (3) | KRAS2, RASK2 |
| Lung cancer, 211980 (3) | PPP2R1B |
| Lung cancer, 211980 (3) | SLC22A1L, BWSCR1A, IMPT1 |
| Lung cancer, somatic, 211980 (3) | MAP3K8, COT, EST, TPL2 |
| Lupus nephritis, susceptibility to (3) | FCGR2A, IGFR2, CD32 |
| Lymphangioleiomyomatosis, 606690 (3) | TSC1, LAM |
| Lymphangioleiomyomatosis, somatic, | TSC2, LAM |
| 606690 (3) | |
| Lymphedema and ptosis, 153000 (3) | FOXC2, FKHL14, MFH1 |
| Lymphedema-distichiasis syndrome, | FOXC2, FKHL14, MFH1 |
| 153400 (3) | |
| Lymphedema-distichiasis syndrome with | FOXC2, FKHL14, MFH1 |
| renal disease and diabetes mellitus (3) | |
| Lymphedema, hereditary I, 153100 (3) | FLT4, VEGFR3, PCL |
| Lymphedema, hereditary II, 153200 (3) | FOXC2, FKHL14, MFH1 |
| Lymphocytic leukemia, acute T-cell (3) | RAP1GDS1 |
| Lymphoma, B-cell non-Hodgkin, somatic (3) | ATM, ATA, AT1 |
| Lymphoma, diffuse large cell (3) | BCL8 |
| Lymphoma, follicular (3) | BCL10 |
| Lymphoma, MALT (3) | BCL10 |
| Lymphoma, mantle cell (3) | ATM, ATA, AT1 |
| Lymphoma, non-Hodgkin (3) | RAD54B |
| Lymphoma, non-Hodgkin (3) | RAD54L, HR54, HRAD54 |
| Lymphoma, progression of (3) | FCGR2B, CD32 |
| Lymphoma, somatic (3) | MAD1L1, TXBP181 |
| Lymphoma, T-cell (3) | MSH2, COCA1, FCC1, HNPCC1 |
| Lymphoproliferative syndrome, X-linked, | SH2D1A, LYP, IMD5, XLP, XLPD |
| 308240 (3) | |
| Lynch cancer family syndrome II, 114400 | MSH2, COCA1, FCC1, HNPCC1 |
| (3) | |
| Lysinuric protein intolerance, 222700 (3) | SLC7A7, LPI |
| Machado-Joseph disease, 109150 (3) | ATXN3, MJD, SCA3 |
| Macrocytic anemia, refractory, of 5q- | IRF1, MAR |
| syndrome, 153550 (3) | |
| Macrothrombocytopenia, 300367 (3) | GATA1, GF1, ERYF1, NFE1 |
| Macular corneal dystrophy, 217800 (3) | CHST6, MCDC1 |
| Macular degeneration, age-related, 1, | HF1, CFH, HUS |
| 603075 (3) | |
| Macular degeneration, age-related, 1, | HMCN1, FBLN6, FIBL6 |
| 603075 (3) | |
| Macular degeneration, age-related, 3, | FBLN5, ARMD3 |
| 608895 (3) | |
| Macular degeneration, juvenile, 248200 (3) | CNGB3, ACHM3 |
| Macular degeneration, X-linked atrophic (3) | RPGR, RP3, CRD, RP15, COD1 |
| Macular dystrophy (3) | RDS, RP7, PRPH2, PRPH, AVMD, |
| | AOFMD |
| Macular dystrophy, age-related, 2, 153800 | ABCA4, ABCR, STGD1, FFM, RP19 |
| (3) | |
| Macular dystrophy, autosomal dominant, | ELOVL4, ADMD, STGD2, STGD3 |
| chromosome 6-linked, 600110 (3) | |
| Macular dystrophy, vitelliform, 608161 (3) | RDS, RP7, PRPH2, PRPH, AVMD, |
| | AOFMD |
| Macular dystrophy, vitelliform type, 153700 | VMD2 |
| (3) | |
| Maculopathy, bull's-eye, 153870 (3) | VMD2 |
| Major depressive disorder and accelerated | FKBP5, FKBP51 |
| response to antidepressant drug treatment, | |
| 608616 (3) | |
| Malaria, cerebral, reduced risk of, 248310 | CD36 |
| (3) | |
| Malaria, cerebral, susceptibility to, 248310 | CD36 |
| (3) | |
| Malaria, cerebral, susceptibility to (3) | ICAM1 |
| Malaria, cerebral, susceptibility to (3) | TNF, TNFA |
| Malaria, resistance to, 248310 (3) | GYPC, GE, GPC |
| Malaria, resistance to, 248310 (3) | NOS2A, NOS2 |
| Malignant hyperthermia susceptibility 1, | RYR1, MHS, CCO |
| 145600 (3) | |
| Malignant hyperthermia susceptibility 5, | CACNA1S, CACNL1A3, CCHL1A3 |
| 601887 (3) | |
| Malonyl-CoA decarboxylase deficiency, | MLYCD, MCD |
| 248360 (3) | |
| MALT lymphoma (3) | MALT1, MLT |
| Mandibuloacral dysplasia with type B | ZMPSTE24, FACE1, STE24, MADB |
| lipodystrophy, 608612 (3) | |
| Mannosidosis, alpha-, types I and II, 248500 | MAN2B1, MANB |
| (3) | |
| Mannosidosis, beta, 248510 (3) | MANBA, MANB1 |
| Maple syrup urine disease, type Ia, 248600 | BCKDHA, MSUD1 |
| (3) | |
| Maple syrup urine disease, type Ib (3) | BCKDHB, E1B |
| Maple syrup urine disease, type II (3) | DBT, BCATE2 |
| Maple syrup urine disease, type III, 248600 | DLD, LAD, PHE3 |
| (3) | |
| Marfan syndrome, 154700 (3) | FBN1, MFS1, WMS |
| Marfan syndrome, atypical (3) | COL1A2 |
| Maroteaux-Lamy syndrome, several forms | ARSB, MPS6 |
| (3) | |
| Marshall syndrome, 154780 (3) | COL11A1, STL2 |
| MASA syndrome, 303350 (3) | L1CAM, CAML1, HSAS1 |
| MASP2 deficiency (3) | MASP2 |
| MASS syndrome, 604308 (3) | FBN1, MFS1, WMS |
| Mast cell leukemia (3) | KIT, PBT |
| Mastocytosis with associated hematologic | KIT, PBT |
| disorder (3) | |
| Mast syndrome, 248900 (3) | ACP33, MAST, SPG21 |
| May-Hegglin anomaly, 155100 (3) | MYH9, MHA, FTNS, DFNA17 |
| McArdle disease, 232600 (3) | PYGM |
| McCune-Albright syndrome, 174800 (3) | GNAS, GNAS1, GPSA, POH, PHP1B, |
| | PHP1A, AHO |
| McKusick-Kaufman syndrome, 236700 (3) | MKKS, HMCS, KMS, MKS, BBS6 |
| McLeod syndrome (3) | XK |
| McLeod syndrome with neuroacanthosis (3) | XK |
| Medullary cystic kidney disease 2, 603860 | UMOD, HNFJ, FJHN, MCKD2, |
| (3) | ADMCKD2 |
| Medullary thyroid carcinoma, 155240 (3) | RET, MEN2A |
| Medullary thyroid carcinoma, familial, | NTRK1, TRKA, MTC |
| 155240 (3) | |
| Medulloblastoma, 155255 (3) | PTCH2 |
| Medulloblastoma, desmoplastic, 155255 (3) | SUFU, SUFUXL, SUFUH |
| Meesmann corneal dystrophy, 122100 (3) | KRT12 |
| Meesmann corneal dystrophy, 122100 (3) | KRT3 |
| Megakaryoblastic leukemia, acute (3) | MKL1, AMKL, MAL |
| Megalencephalic leukoencephalopathy with | MLC1, LVM, VL |
| subcortical cysts, 604004 (3) | |
| Megaloblastic anemia-1, Finnish type, | CUBN, IFCR, MGA1 |
| 261100 (3) | |
| Megaloblastic anemia-1, Norwegian type, | AMN |
| 261100 (3) | |
| Melanoma (3) | CDK4, CMM3 |
| Melanoma and neural system tumor | CDKN2A, MTS1, P16, MLM, CMM2 |
| syndrome, 155755 (3) | |
| Melanoma, cutaneous malignant, 2, 155601 | CDKN2A, MTS1, P16, MLM, CMM2 |
| (3) | |
| Melanoma, cutaneous malignant, | XRCC3 |
| susceptibility to (3) | |
| Melanoma, malignant sporadic (3) | STK11, PJS, LKB1 |
| Melanoma, melignant, somatic (3) | BRAF |
| Meleda disease, 248300 (3) | SLURP1, MDM |
| Melnick-Needles syndrome, 309350 (3) | FLNA, FLN1, ABPX, NHBP, OPD1, |
| | OPD2, FMD, MNS |
| Melorheostosis with osteopoikilosis, 155950 | LEMD3, MAN1 |
| (3) | |
| Memory impairment, susceptibility to (3) | BDNF |
| Meniere disease 156000 (3) ( ) | COCH, DFNA9 |
| Meningioma, 607174 (3) | MN1, MGCR |
| Meningioma, 607174 (3) | PTEN, MMAC1 |
| Meningioma, NF2-related, somatic, 607174 | NF2 |
| (3) | |
| Meningioma, SIS-related (3) | PDGFB, SIS |
| Meningococcal disease, susceptibility to (3) | MBL2, MBL, MBP1 |
| Menkes disease, 309400 (3) | ATP7A, MNK, MK, OHS |
| Mental retardation, nonsyndromic, | PRSS12, BSSP3 |
| autosomal recessive, 249500 (3) | |
| Mental retardation, nonsyndromic, | CRBN, MRT2A |
| autosomal recessive, 2A, 607417 (3) | |
| Mental retardation, X-linked, 300425 (3) | NLGN4, KIAA1260, AUTSX2 |
| Mental retardation, X-linked, 300458 (3) | MECP2, RTT, PPMX, MRX16, MRX79 |
| Mental retardation, X-linked 30, 300558 (3) | PAK3, MRX30, MRX47 |
| Mental retardation, X-linked, 34, 300426 (3) | IL1RAPL, MRX34 |
| Mental retardation, X-linked 36, 300430 (3) | ARX, ISSX, PRTS, MRXS1, MRX36, |
| | MRX54 |
| Mental retardation, X-linked (3) | SLC6A8, CRTR |
| Mental retardation, X-linked-44, 300501 (3) | FTSJ1, JM23, SPB1, MRX44, MRX9 |
| Mental retardation, X-linked 45, 300498 (3) | ZNF81, MRX45 |
| Mental retardation, X-linked 54, 300419 (3) | ARX, ISSX, PRTS, MRXS1, MRX36, |
| | MRX54 |
| Mental retardation, X-linked 58, 300218 (3) | TM4SF2, MXS1, A15 |
| Mental retardation, X-linked, 60, 300486 (3) | OPHN1 |
| Mental retardation, X-linked-9, 309549 (3) | FTSJ1, JM23, SPB1, MRX44, MRX9 |
| Mental retardation, X-linked, FRAXE type | FMR2, FRAXE, MRX2 |
| (3) | |
| Mental retardation, X-linked, JARID1C- | SMCX, MRXJ, DXS1272E, XE169, |
| related, 300534 (3) | JARID1C |
| Mental retardation, X-linked nonspecific, | GDI1, RABGD1A, MRX41, MRX48 |
| 309541 (3) | |
| Mental retardation, X-linked nonspecific, 63, | FACL4, ACS4, MRX63 |
| 300387 (3) | |
| Mental retardation, X-linked nonspecific, | RPS6KA3, RSK2, MRX19 |
| type 19 (3) | |
| Mental retardation, X-linked nonspecific, | ARHGEF6, MRX46, COOL2 |
| type 46, 300436 (3) | |
| Mental retardation, X-linked nonsyndromic | AGTR2 |
| (3) | |
| Mental retardation, X-linked nonsyndromic | FGD1, FGDY, AAS |
| (3) | |
| Mental retardation, X-linked nonsyndromic | ZNF41 |
| (3) | |
| Meesmann corneal dystrophy, 122100 (3) | KRT12 |
| Meesmann corneal dystrophy, 122100 (3) | KRT3 |
| Megakaryoblastic leukemia, acute (3) | MKL1, AMKL, MAL |
| Megalencephalic leukoencephalopathy with | MLC1, LVM, VL |
| subcortical cysts, 604004 (3) | |
| Megaloblastic anemia-1, Finnish type, | CUBN, IFCR, MGA1 |
| 261100 (3) | |
| Megaloblastic anemia-1, Norwegian type, | AMN |
| 261100 (3) | |
| Melanoma (3) | CDK4, CMM3 |
| Melanoma and neural system tumor | CDKN2A, MTS1, P16, MLM, CMM2 |
| syndrome, 155755 (3) | |
| Melanoma, cutaneous malignant, 2, 155601 | CDKN2A, MTS1, P16, MLM, CMM2 |
| (3) | |
| Melanoma, cutaneous malignant, | XRCC3 |
| susceptibility to (3) | |
| Melanoma, malignant sporadic (3) | STK11, PJS, LKB1 |
| Melanoma, melignant, somatic (3) | BRAF |
| Meleda disease, 248300 (3) | SLURP1, MDM |
| Melnick-Needles syndrome, 309350 (3) | FLNA, FLN1, ABPX, NHBP, OPD1, |
| | OPD2, FMD, MNS |
| Melorheostosis with osteopoikilosis, 155950 | LEMD3, MAN1 |
| (3) | |
| Memory impairment, susceptibility to (3) | BDNF |
| Meniere disease 156000 (3) ( ) | COCH, DFNA9 |
| Meningioma, 607174 (3) | MN1, MGCR |
| Meningioma, 607174 (3) | PTEN, MMAC1 |
| Meningioma, NF2-related, somatic, 607174 | NF2 |
| (3) | |
| Meningioma, SIS-related (3) | PDGFB, SIS |
| Meningococcal disease, susceptibility to (3) | MBL2, MBL, MBP1 |
| Menkes disease, 309400 (3) | ATP7A, MNK, MK, OHS |
| Mental retardation, nonsyndromic, | PRSS12, BSSP3 |
| autosomal recessive, 249500 (3) | |
| Mental retardation, nonsyndromic, | CRBN, MRT2A |
| autosomal recessive, 2A, 607417 (3) | |
| Mental retardation, X-linked, 300425 (3) | NLGN4, KIAA1260, AUTSX2 |
| Mental retardation, X-linked, 300458 (3) | MECP2, RTT, PPMX, MRX16, MRX79 |
| Mental retardation, X-linked 30, 300558 (3) | PAK3, MRX30, MRX47 |
| Mental retardation, X-linked, 34, 300426 (3) | IL1RAPL, MRX34 |
| Mental retardation, X-linked 36, 300430 (3) | ARX, ISSX, PRTS, MRXS1, MRX36, |
| | MRX54 |
| Mental retardation, X-linked (3) | SLC6A8, CRTR |
| Mental retardation, X-linked-44, 300501 (3) | FTSJ1, JM23, SPB1, MRX44, MRX9 |
| Mental retardation, X-linked 45, 300498 (3) | ZNF81, MRX45 |
| Mental retardation, X-linked 54, 300419 (3) | ARX, ISSX, PRTS, MRXS1, MRX36, |
| | MRX54 |
| Mental retardation, X-linked 58, 300218 (3) | TM4SF2, MXS1, A15 |
| Mental retardation, X-linked, 60, 300486 (3) | OPHN1 |
| Mental retardation, X-linked-9, 309549 (3) | FTSJ1, JM23, SPB1, MRX44, MRX9 |
| Mental retardation, X-linked, FRAXE type | FMR2, FRAXE, MRX2 |
| (3) | |
| Mental retardation, X-linked, JARID1C- | SMCX, MRXJ, DXS1272E, XE169, |
| related, 300534 (3) | JARID1C |
| Mental retardation, X-linked nonspecific, | GDI1, RABGD1A, MRX41, MRX48 |
| 309541 (3) | |
| Mental retardation, X-linked nonspecific, 63, | FACL4, ACS4, MRX63 |
| 300387 (3) | |
| Mental retardation, X-linked nonspecific, | RPS6KA3, RSK2, MRX19 |
| type 19 (3) | |
| Mental retardation, X-linked nonspecific, | ARHGEF6, MRX46, COOL2 |
| type 46, 300436 (3) | |
| Mental retardation, X-linked nonsyndromic | AGTR2 |
| (3) | |
| Mental retardation, X-linked nonsyndromic | FGD1, FGDY, AAS |
| (3) | |
| Mental retardation, X-linked nonsyndromic | ZNF41 |
| (3) | |
| Mental retardation, X-linked nonsyndromic, | DLG3, NEDLG, SAP102, MRX |
| DLG3-related (3) | |
| Mental retardation, X-linked, Snyder- | SMS, SRS, MRSR |
| Robinson type, 309583 (3) | |
| Mental retardation, X-linked, with isolated | SOX3, MRGH |
| growth hormone deficiency, 300123 (3) | |
| Mental retardation, X-linked, with | MECP2, RTT, PPMX, MRX16, MRX79 |
| progressive spasticity, 300279 (3) | |
| Mental retardation, X-linked, with seizures | SLC6A8, CRTR |
| and carrier manifestations, 300397 (3) | |
| Mephenytoin poor metabolizer (3) | CYP2C, CYP2C19 |
| Merkel cell carcinoma, somatic (3) | SDHD, PGL1 |
| Mesangial sclerosis, isolated diffuse, | WT1 |
| 256370 (3) | |
| Mesothelioma (3) | BCL10 |
| Metachromatic leukodystrophy, 250100 (3) | ARSA |
| Metachromatic leukodystrophy due to | PSAP, SAP1 |
| deficiency of SAP-1 (3) | |
| Metaphyseal chondrodysplasia, Murk | PTHR1, PTHR |
| Jansen type, 156400 (3) | |
| Metaphyseal chondrodysplasia, Schmid | COL10A1 |
| type (3) | |
| Metaphyseal dysplasia without | RMRP, RMRPR, CHH |
| hypotrichosis, 250460 (3) | |
| Methemoglobinemia due to cytochrome b5 | CYB5 |
| deficiency (3) | |
| Methemoglobinemias, alpha-(3) | HBA1 |
| Methemoglobinemias, beta-(3) | HBB |
| Methemoglobinemia, type I (3) | DIA1 |
| Methemoglobinemia, type II (3) | DIA1 |
| Methionine adenosyltransferase deficiency, | MAT1A, MATA1, SAMS1 |
| autosomal recessive (3) | |
| Methylcobalamin deficiency, cblG type, | MTR |
| 250940 (3) | |
| Methylmalonate semialdehyde | ALDH6A1, MMSDH |
| dehydrogenase deficiency (3) | |
| Methylmalonic aciduria, mut(0) type, 251000 | MUT, MCM |
| (3) | |
| Methylmalonic aciduria, vitamin B12- | MMAA |
| responsive, 251100 (3) | |
| Methylmalonic aciduria, vitamin B12- | MMAB |
| responsive, due to defect in synthesis of | |
| adenosylcobalamin, cblB complementation | |
| type, 251110 (3) | |
| Mevalonicaciduria (3) | MVK, MVLK |
| MHC class II deficiency, complementation | RFXANK |
| group B, 209920 (3) | |
| Microcephaly, Amish type, 607196 (3) | SLC25A19, DNC, MUP1, MCPHA |
| Microcephaly, autosomal recessive 1, | MCPH1 |
| 251200 (3) | |
| Microcephaly, primary autosomal recessive, | CDK5RAP2, KIAA1633, MCPH3 |
| 3, 604804 (3) | |
| Microcephaly, primary autosomal recessive, | ASPM, MCPH5 |
| 5, 608716 (3) | |
| Microcephaly, primary autosomal recessive, | CEMPJ, CPAP, MCPH6 |
| 6, 608393 (3) | |
| Microcoria-congenital nephrosis syndrome, | LAMB2, LAMS |
| 609049 (3) | |
| Micropenis (3) | LHCGR |
| Microphthalmia, cataracts, and iris | CHX10, HOX10 |
| abnormalities (3) | |
| Microphthalmia, SIX6-related (3) | SIX6 |
| Microphthalmia with associated anomalies | BCOR, KIAA1575, MAA2, ANOP2 |
| 2, 300412 (3) | |
| Migraine, familial hemiplegic, 2, 602481 (3) | ATP1A2, FHM2, MHP2 |
| Migraine, resistance to, 157300 (3) | EDNRA |
| Migraine, susceptibility to, 157300 (3) | ESR1, ESR |
| Migraine without aura, susceptibility to, | TNF, TNFA |
| 157300 (3) | |
| Miller-Dieker lissencephaly, 247200 (3) | YWHAE, MDCR, MDS |
| Mitochondrial complex I deficiency, 252010 | NDUFS1 |
| (3) | |
| Mitochondrial complex I deficiency, 252010 | NDUFS2 |
| (3) | |
| Mitochondrial complex I deficiency, 252010 | NDUFS4, AQDQ |
| (3) | |
| Mitochondrial complex I deficiency, 252010 | NDUFV1, UQOR1 |
| (3) | |
| Mitochondrial complex III deficiency, 124000 | BCS1L, FLNMS, GRACILE |
| (3) | |
| Mitochondrial complex III deficiency, 124000 | UQCRB, UQBP, QPC |
| (3) | |
| Mitochondrial DNA depletion myopathy, | TK2 |
| 251880 (3) | |
| Mitochondrial DNA depletion syndrome, | SUCLA2 |
| 251880 (3) | |
| Mitochondrial DNA-depletion syndrome, | DGUOK, DGK |
| hepatocerebral form, 251880 (3) | |
| Mitochondrial myopathy and sideroblastic | PUS1, MLASA |
| anemia, 600462 (3) | |
| Mitochondrial respiratory chain complex II | SDHA, SDH2, SDHF |
| deficiency, 252011 (3) | |
| Miyoshi myopathy, 254130 (3) | DYSF, LGMD2B |
| MODY5 with nephron agenesis (3) | TCF2, HNF2 |
| MODY5 with non-diabetic renal disease and | TCF2, HNF2 |
| Mullerian aplasia (3) | |
| MODY, one form, 125850 (3) | INS |
| MODY, type I, 125850 (3) | HNF4A, TCF14, MODY1 |
| MODY, type II, 125851 (3) | GCK |
| MODY, type III, 600496 (3) | TCF1, HNF1A, MODY3 |
| MODY, type IV (3) | IPF1 |
| MODY, type V, 604284 (3) | TCF2, HNF2 |
| Mohr-Tranebjaerg syndrome, 304700 (3) | TIMM8A, DFN1, DDP, MTS, DDP1 |
| Molybdenum cofactor deficiency, type A, | MOCS1, MOCOD |
| 252150 (3) | |
| Molybdenum cofactor deficiency, type B, | MOCS2, MPTS |
| 252150 (3) | |
| Molybdenum cofactor deficiency, type C, | GPH, KIAA1385, GEPH |
| 252150 (3) | |
| Monilethrix, 158000 (3) | KRTHB1, HB1 |
| Monilethrix, 158000 (3) | KRTHB6, HB6 |
| Morning glory disc anomaly (3) | PAX6, AN2, MGDA |
| Mowat-Wilson syndrome, 235730 (3) | ZFHX1B, SMADIP1, SIP1 |
| Moyamoya disease 3 (3) | MYMY3 |
| Muckle-Wells syndrome, 191900 (3) | CIAS1, C1orf7, FCU, FCAS |
| Mucoepidermoid salivary gland carcinoma | MAML2, MAM3 |
| (3) | |
| Mucoepidermoid salivary gland carcinoma | MECT1, KIAA0616 |
| (3) | |
| Mucolipidosis IIIA, 252600 (3) | GNPTAB, GNPTA |
| Mucolipidosis IIIC, 252605 (3) | GNPTAG |
| Mucolipidosis IV, 252650 (3) | MCOLN1, ML4 |
| Mucopolysaccharidosis Ih, 607014 (3) | IDUA, IDA |
| Mucopolysaccharidosis Ih/s, 607015 (3) | IDUA, IDA |
| Mucopolysaccharidosis II (3) | IDS, MPS2, SIDS |
| Mucopolysaccharidosis Is, 607016 (3) | IDUA, IDA |
| Mucopolysaccharidosis IVA (3) | GALNS, MPS4A |
| Mucopolysaccharidosis IVB (3) | GLB1 |
| Mucopolysaccharidosis type IIID, 252940 | GNS, G6S |
| (3) | |
| Mucopolysaccharidosis type IX, 601492 (3) | HYAL1 |
| Mucopolysaccharidosis VII (3) | GUSB, MPS7 |
| Muenke syndrome, 602849 (3) | FGFR3, ACH |
| Muir-Torre syndrome, 158320 (3) | MLH1, COCA2, HNPCC2 |
| Muir-Torre syndrome, 158320 (3) | MSH2, COCA1, FCC1, HNPCC1 |
| Mulibrey nanism, 253250 (3) | TRIM37, MUL, KIAA0898 |
| Multiple cutaneous and uterine | FH |
| leiomyomata, 150800 (3) | |
| Multiple endocrine neoplasia I (3) | MEN1 |
| Multiple endocrine neoplasia IIA, 171400 (3) | RET, MEN2A |
| Multiple endocrine neoplasia IIB, 162300 (3) | RET, MEN2A |
| Multiple malignancy syndrome (3) | TP53, P53, LFS1 |
| Multiple myeloma (3) | IRF4, LSIRF |
| Multiple myeloma, resistance to, 254500 (3) | LIG4 |
| Multiple sclerosis, susceptibility to, 126200 | MHC2TA, C2TA |
| (3) | |
| Multiple sclerosis, susceptibility to, 126200 | PTPRC, CD45, LCA |
| (3) | |
| Multiple sulfatase deficiency, 272200 (3) | SUMF1, FGE |
| Muscle-eye-brain disease, 253280 (3) | POMGNT1, MEB |
| Muscle glycogenosis (3) | PHKA1 |
| Muscle hypertrophy (3) | GDF8, MSTN |
| Muscular dystrophy, congenital, 1C (3) | FKRP, MDC1C, LGMD2I |
| Muscular dystrophy, congenital, due to | LAMA2, LAMM |
| partial LAMA2 deficiency, 607855 (3) | |
| Muscular dystrophy, congenital merosin- | LAMA2, LAMM |
| deficient, 607855 (3) | |
| Muscular dystrophy, congenital, type 1D, | LARGE, KIAA0609, MDC1D |
| 608840 (3) | |
| Muscular dystrophy, Fukuyama congenital, | FCMD |
| 253800 (3) | |
| Muscular dystrophy, limb-girdle, type 1A, | TTID, MYOT |
| 159000 (3) | |
| Muscular dystrophy, limb-girdle, type 2A, | CAPN3, CANP3 |
| 253600 (3) | |
| Muscular dystrophy, limb-girdle, type 2B, | DYSF, LGMD2B |
| 253601 (3) | |
| Muscular dystrophy, limb-girdle, type 2C, | SGCG, LGMD2C, DMDA1, SCG3 |
| 253700 (3) | |
| Muscular dystrophy, limb-girdle, type 2D, | SGCA, ADL, DAG2, LGMD2D, DMDA2 |
| 608099 (3) | |
| Muscular dystrophy, limb-girdle, type 2E, | SGCB, LGMD2E |
| 604286 (3) | |
| Muscular dystrophy, limb-girdle, type 2F, | SGCD, SGD, LGMD2F, CMD1L |
| 601287 (3) | |
| Muscular dystrophy, limb-girdle, type 2G, | TCAP, LGMD2G, CMD1N |
| 601954 (3) | |
| Muscular dystrophy, limb-girdle, type 2H, | TRIM32, HT2A, LGMD2H |
| 254110 (3) | |
| Muscular dystrophy, limb-girdle, type 2I, | FKRP, MDC1C, LGMD2I |
| 607155 (3) | |
| Muscular dystrophy, limb-girdle, type 2J, | TTN, CMD1G, TMD, LGMD2J |
| 608807 (3) | |
| Muscular dystrophy, limb-girdle, type 2K, | POMT1 |
| 609308 (3) | |
| Muscular dystrophy, limb-girdle, type IC, | CAV3, LGMD1C |
| 607801 (3) | |
| Muscular dystrophy, rigid spine, 1, 602771 | SEPN1, SELN, RSMD1 |
| (3) | |
| Muscular dystrophy with epidermolysis | PLEC1, PLTN, EBS1 |
| bullosa simplex, 226670 (3) | |
| Myasthenia, familial infantile, 1, 605809 (3) | CMS1A1, FIM1 |
| Myasthenic syndrome (3) | SCN4A, HYPP, NAC1A |
| Myasthenic syndrome, congenital, | CHRNB1, ACHRB, SCCMS, CMS2A, |
| associated with acetylcholine receptor | CMS1D |
| deficiency, 608931 (3) | |
| Myasthenic syndrome, congenital, | CHRNE, SCCMS, CMS2A, FCCMS, |
| associated with acetylcholine receptor | CMS1E, CMS1D |
| deficiency, 608931 (3) | |
| Myasthenic syndrome, congenital, | RAPSN, CMS1D, CMS1E |
| associated with acetylcholine receptor | |
| deficiency, 608931 (3) | |
| Myasthenic syndrome, congenital, | CHAT, CMS1A2 |
| associated with episodic apnea, 254210 (3) | |
| Myasthenic syndrome, congenital, | RAPSN, CMS1D, CMS1E |
| associated with facial dysmorphism and | |
| acetylcholine receptor deficiency, 608931 (3) | |
| Myasthenic syndrome, fast-channel | CHRNA1, ACHRD, CMS2A, SCCMS, |
| congenital, 608930 (3) | FCCMS |
| Myasthenic syndrome, fast-channel | CHRND, ACHRD, SCCMS, CMS2A, |
| congenital, 608930 (3) | FCCMS |
| Myasthenic syndrome, fast-channel | CHRNE, SCCMS, CMS2A, FCCMS, |
| congenital, 608930 (3) | CMS1E, CMS1D |
| Myasthenic syndrome, slow-channel | CHRNA1, ACHRD, CMS2A, SCCMS, |
| congenital, 601462 (3) | FCCMS |
| Myasthenic syndrome, slow-channel | CHRNB1, ACHRB, SCCMS, CMS2A, |
| congenital, 601462 (3) | CMS1D |
| Myasthenic syndrome, slow-channel | CHRND, ACHRD, SCCMS, CMS2A, |
| congenital, 601462 (3) | FCCMS |
| Myasthenic syndrome, slow-channel | CHRNE, SCCMS, CMS2A, FCCMS, |
| congenital, 601462 (3) | CMS1E, CMS1D |
| Mycobacterial and salmonella infections, | IL12RB1 |
| susceptibility to, 209950 (3) | |
| Mycobacterial infection, atypical, familial | IFNGR1 |
| disseminated, 209950 (3) | |
| Mycobacterial infection, atypical, familial | IFNGR2, IFNGT1, IFGR2 |
| disseminated, 209950 (3) | |
| Mycobacterial infection, atypical, familial | STAT1 |
| disseminated, 209950 (3) | |
| Mycobacterium tuberculosis, suceptibility to | NRAMP1, NRAMP |
| infection by, 607948 (3) | |
| Myelodysplasia syndrome-1 (3) | MDS1 |
| Myelodysplastic syndrome (3) | FACL6, ACS2 |
| Myelodysplastic syndrome, preleukemic (3) | IRF1, MAR |
| Myelofibrosis, idiopathic, 254450 (3) | JAK2 |
| Myelogenous leukemia, acute (3) | FACL6, ACS2 |
| Myelogenous leukemia, acute (3) | IRF1, MAR |
| Myeloid leukemia, acute, M4Eo subtype (3) | CBFB |
| Myeloid malignancy, predisposition to (3) | CSF1R, FMS |
| Myelokathexis, isolated (3) | CXCR4, D2S201E, NPY3R, WHIM |
| Myelomonocytic leukemia, chronic (3) | PDGFRB, PDGFR |
| Myeloperoxidase deficiency, 254600 (3) | MPO |
| Myeloproliferative disorder with eosinophilia, | PDGFRB, PDGFR |
| 131440 (3) | |
| Myoadenylate deaminase deficiency (3) | AMPD1 |
| Myocardial infarction, decreased | F7 |
| susceptibility to (3) | |
| Myocardial infarction susceptibility (3) | APOE, AD2 |
| Myocardial infarction, susceptibility to (3) | ACE, DCP1, ACE1 |
| Myocardial infarction, susceptibility to (3) | ALOX5AP, FLAP |
| Myocardial infarction, susceptibility to (3) | LGALS2 |
| Myocardial infarction, susceptibility to (3) | LTA, TNFB |
| Myocardial infarction, susceptibility to (3) | OLR1, LOX1 |
| Myocardial infarction, susceptibility to (3) | THBD, THRM |
| Myocardial infarction, susceptibility to, | GCLM, GLCLR |
| 608446 (3) | |
| Myocardial infarction, susceptibility to, | TNFSF4, GP34, OX4OL |
| 608446 (3) | |
| Myoclonic epilepsy, juvenile, 1, 254770 (3) | EFHC1, FLJ10466, EJM1 |
| Myoclonic epilepsy, severe, of infancy, | GABRG2, GEFSP3, CAE2, ECA2 |
| 607208 (3) | |
| Myoclonic epilepsy with mental retardation | ARX, ISSX, PRTS, MRXS1, MRX36, |
| and spasticity, 300432 (3) | MRX54 |
| Myoglobinuria/hemolysis due to PGK | PGK1, PGKA |
| deficiency (3) | |
| Myokymia with neonatal epilepsy, 606437 | KCNQ2, EBN1 |
| (3) | |
| Myoneurogastrointestinal | ECGF1 |
| encephalomyopathy syndrome, 603041 (3) | |
| Myopathy, actin, congenital, with cores (3) | ACTA1, ASMA, NEM3, NEM1 |
| Myopathy, actin, congenital, with excess of | ACTA1, ASMA, NEM3, NEM1 |
| thin myofilaments, 161800 (3) | |
| Myopathy, cardioskeletal, desmin-related, | CRYAB, CRYA2, CTPP2 |
| with cataract, 608810 (3) | |
| Myopathy, centronuclear, 160150 (3) | MYF6 |
| Myopathy, congenital (3) | ITGA7 |
| Myopathy, desmin-related, cardioskeletal, | DES, CMD1I |
| 601419 (3) | |
| Myopathy, distal, with anterior tibial onset, | DYSF, LGMD2B |
| 606768 (3) | |
| Myopathy, distal, with decreased caveolin 3 | CAV3, LGMD1C |
| (3) | |
| Myopathy due to CPT II deficiency, 255110 | CPT2 |
| (3) | |
| Myopathy due to phosphoglycerate mutase | PGAM2, PGAMM |
| deficiency (3) | |
| Myopathy, Laing distal, 160500 (3) | MYH7, CMH1, MPD1 |
| Myopathy, myosin storage, 608358 (3) | MYH7, CMH1, MPD1 |
| Myopathy, nemaline, 3, 161800 (3) | ACTA1, ASMA, NEM3, NEM1 |
| Myotilinopathy, 609200 (3) | TTID, MYOT |
| Myotonia congenita, atypical, | SCN4A, HYPP, NAC1A |
| acetazolamide-responsive, 608390 (3) | |
| Myotonia congenita, dominant, 160800 (3) | CLCN1 |
| Myotonia congenita, recessive, 255700 (3) | CLCN1 |
| Myotonia levior, recessive (3) | CLCN1 |
| Myotonic dystrophy, 160900 (3) | DMPK, DM, DMK |
| Myotonic dystrophy, type 2, 602668 (3) | ZNF9, CNBP1, DM2, PROMM |
| Myotubular myopathy, X-linked, 310400 (3) | MTM1, MTMX |
| Myxoid liposarcoma (3) | DDIT3, GADD153, CHOP10 |
| Myxoma, intracardiac, 255960 (3) | PRKAR1A, TSE1, CNC1, CAR |
| N-acetylglutamate synthase deficiency, | NAGS |
| 237310 (3) | |
| Nail-patella syndrome, 161200 (3) | LMX1B, NPS1 |
| Nail-patella syndrome with open-angle | LMX1B, NPS1 |
| glaucoma, 137750 (3) | |
| Nance-Horan syndrome, 302350 (3) | NHS |
| Narcolepsy, 161400 (3) | HCRT, OX |
| Nasopharyngeal carcinoma, 161550 (3) | TP53, P53, LFS1 |
| Nasu-Hakola disease, 221770 (3) | TREM2 |
| Nasu-Hakola disease, 221770 (3) | TYROBP, PLOSL, DAP12 |
| Naxos disease, 601214 (3) | JUP, DP3, PDGB |
| Nemaline myopathy, 161800 (3) | TPM2, TMSB, AMCD1, DA1 |
| Nemaline myopathy 1, autosomal dominant, | TPM3, NEM1 |
| 161800 (3) | |
| Nemaline myopathy 2, autosomal recessive, | NEB, NEM2 |
| 256030 (3) | |
| Nemaline myopathy, Amish type, 605355 | TNNT1, ANM |
| (3) | |
| Neonatal ichthyosis-sclerosing cholangitis | CLDN1, SEMP1 |
| syndrome, 607626 (3) | |
| Nephrogenic syndrome of inappropriate | AVPR2, DIR, DI1, ADHR |
| antidiuresis, 300539 (3) | |
| Nephrolithiasis, type I, 310468 (3) | CLCN5, CLCK2, NPHL2, DENTS |
| Nephrolithiasis, uric acid, susceptibility to, | ZNF365, UAN |
| 605990 (3) | |
| Nephronophthisis 2, infantile, 602088 (3) | INVS, INV, NPHP2, NPH2 |
| Nephronophthisis 4, 606966 (3) | NPHP4, SLSN4 |
| Nephronophthisis, adolescent, 604387 (3) | NPHP3, NPH3 |
| Nephronophthisis, juvenile, 256100 (3) | NPHP1, NPH1, SLSN1 |
| Nephropathy, chronic hypocomplementemic | HF1, CFH, HUS |
| (3) | |
| Nephropathy with pretibial epidermolysis | CD151, PETA3, SFA1 |
| bullosa and deafness, 609057 (3) | |
| Nephrosis-1, congenital, Finnish type, | NPHS1, NPHN |
| 256300 (3) | |
| Nephrotic syndrome, steroid-resistant, | PDCN, NPHS2, SRN1 |
| 600995 (3) | |
| Netherton syndrome, 256500 (3) | SPINK5, LEKTI |
| Neural tube defects, maternal risk of, | MTHFD, MTHFC |
| 601634 (3) | |
| Neuroblastoma, 256700 (3) | NME1, NM23 |
| Neuroblastoma, 256700 (3) | PMX2B, NBPHOX, PHOX2B |
| Neurodegeneration, pantothenate kinase- | PANK2, NBIA1, PKAN, HARP |
| associated, 234200 (3) | |
| Neuroectodermal tumors, supratentorial | PMS2, PMSL2, HNPCC4 |
| primitive, with cafe-au-lait spots, 608623 (3) | |
| Neurofibromatosis, familial spinal, 162210 | NF1, VRNF, WSS, NFNS |
| (3) | |
| Neurofibromatosis-Noonan syndrome, | NF1, VRNF, WSS, NFNS |
| 601321 (3) | |
| Neurofibromatosis, type 1 (3) | NF1, VRNF, WSS, NFNS |
| Neurofibromatosis, type 2, 101000 (3) | NF2 |
| Neurofibromatosis, type I, with leukemia, | MSH2, COCA1, FCC1, HNPCC1 |
| 162200 (3) | |
| Neurofibrosarcoma (3) | MXI1 |
| Neuropathy, congenital hypomyelinating, 1, | EGR2, KROX20 |
| 605253 (3) | |
| Neuropathy, congenital hypomyelinating, | MPZ, CMT1B, CMTDI3, CHM, DSS |
| 605253 (3) | |
| Neuropathy, distal hereditary motor, 608634 | HSPB1, HSP27, CMT2F |
| (3) | |
| Neuropathy, distal hereditary motor, type II, | HSPB8, H11, E2IG1, DHMN2 |
| 158590 (3) | |
| Neuropathy, hereditary sensory and | SPTLC1, LBC1, SPT1, HSN1, HSAN |
| autonomic, type 1, 162400 (3) | |
| Neuropathy, hereditary sensory and | NGFB, HSAN5 |
| autonomic, type V, 608654 (3) | |
| Neuropathy, hereditary sensory, type II, | HSN2 |
| 201300 (3) | |
| Neuropathy, recurrent, with pressure | PMP22, CMT1A, CMT1E, DSS |
| palsies, 162500 (3) | |
| Neutropenia, alloimmune neonatal (3) | FCGR3A, CD16, IGFR3 |
| Neutropenia, congenital, 202700 (3) | ELA2 |
| Neutropenia, severe congenital, 202700 (3) | GFI1, ZNF163 |
| Neutropenia, severe congenital, X-linked, | WAS, IMD2, THC |
| 300299 (3) | |
| Neutrophil immunodeficiency syndrome, | RAC2 |
| 608203 (3) | |
| Nevo syndrome, 601451 (3) | PLOD, PLOD1 |
| Nevus, epidermal, epidermolytic | KRT10 |
| hyperkeratotic type, 600648 (3) | |
| Newfoundland rod-cone dystrophy, 607476 | RLBP1 |
| (3) | |
| Nicotine addiction, protection from (3) | CYP2A6, CYP2A3, CYP2A, P450C2A |
| Nicotine addiction, susceptibility to, 188890 | CHRNA4, ENFL1 |
| (3) | |
| Nicotine dependence, susceptibility to, | GPR51, GABBR2 |
| 188890 (3) | |
| Niemann-Pick disease, type A, 257200 (3) | SMPD1, NPD |
| Niemann-Pick disease, type B, 607616 (3) | SMPD1, NPD |
| Niemann-Pick disease, type C1, 257220 (3) | NPC1, NPC |
| Niemann-pick disease, type C2, 607625 (3) | NPC2, HE1 |
| Niemann-Pick disease, type D, 257220 (3) | NPC1, NPC |
| Night blindness, congenital stationary (3) | GNAT1 |
| Night blindness, congenital stationary, type | CSNB1, NYX |
| 1, 310500 (3) | |
| Night blindness, congenital stationary, type | PDE6B, PDEB, CSNB3 |
| 3, 163500 (3) | |
| Night blindness, congenital stationary, X- | CACNA1F, CSNB2 |
| linked, type 2, 300071 (3) | |
| Night blindness, congenital stationery, | RHO, RP4, OPN2 |
| rhodopsin-related (3) | |
| Nijmegen breakage syndrome, 251260 (3) | NBS1, NBS |
| Nonaka myopathy, 605820 (3) | GNE, GLCNE, IBM2, DMRV, NM |
| Noncompaction of left ventricular | TAZ, EFE2, BTHS, CMD3A, LVNCX |
| myocardium, isolated, 300183 (3) | |
| Non-Hodgkin lymphoma, somatic, 605027 | CASP10, MCH4, ALPS2 |
| (3) | |
| Nonsmall cell lung cancer (3) | IRF1, MAR |
| Nonsmall cell lung cancer, response to | EGFR |
| tyrosine kinase inhibitor in, 211980 (3) | |
| Nonsmall cell lung cancer, somatic (3) | BRAF |
| Noonan syndrome 1, 163950 (3) | PTPN11, PTP2C, SHP2, NS1 |
| Norrie disease (3) | NDP, ND |
| Norum disease, 245900 (3) | LCAT |
| Norwalk virus infection, resistance to (3) | FUT2, SE |
| Nucleoside phosphorylase deficiency, | NP |
| immunodeficiency due to (3) | |
| Obesity, adrenal insufficiency, and red hair | POMC |
| (3) | |
| Obesity, autosomal dominant, 601665 (3) | MC4R |
| Obesity, hyperphagia, and developmental | AKR1C2, DDH2, DD2, HAKRD |
| delay (3) | |
| Obesity, hyperphagia, and developmental | NTRK2, TRKB |
| delay (3) | |
| Obesity, late-onset, 601665 (3) | AGRP, ART, AGRT |
| Obesity, mild, early-onset, 601665 (3) | NR0B2, SHP |
| Obesity, morbid, with hypogonadism (3) | LEP, OB |
| Obesity, morbid, with hypogonadism (3) | LEPR, OBR |
| Obesity, resistance to (3) | PPARG, PPARG1, PPARG2 |
| Obesity, severe, 601665 (3) | PPARG, PPARG1, PPARG2 |
| Obesity, severe, 601665 (3) | SIM1 |
| Obesity, severe, and type II diabetes, | UCP3 |
| 601665 (3) | |
| Obesity, severe, due to leptin deficiency (3) | LEP, OB |
| Obesity, severe, susceptibility to, 601665 (3) | MC3R |
| Obesity, susceptibility to, 300306 (3) | SLC6A14, OBX |
| Obesity, susceptibility to, 601665 (3) | ADRB2 |
| Obesity, susceptibility to, 601665 (3) | ADRB3 |
| Obesity, susceptibility to, 601665 (3) | CART |
| Obesity, susceptibility to, 601665 (3) | ENPP1, PDNP1, NPPS, M6S1, PCA1 |
| Obesity, susceptibility to, 601665 (3) | GHRL |
| Obesity, susceptibility to, 601665 (3) | UCP1 |
| Obesity, susceptibility to, 601665 (3) | UCP2 |
| Obestiy with impaired prohormone | PCSK1, NEC1, PC1, PC3 |
| processing, 600955 (3) | |
| Obsessive-compulsive disorder 1, 164230 | SLC6A4, HTT, OCD1 |
| (3) | |
| Obsessive-compulsive disorder, protection | BDNF |
| against, 164230 (3) | |
| Obsessive-compulsive disorder, | HTR2A |
| susceptibility to, 164230 (3) | |
| Occipital horn syndrome, 304150 (3) | ATP7A, MNK, MK, OHS |
| Ocular albinism, Nettleship-Falls type (3) | OA1 |
| Oculocutaneous albinism, type II, modifier of | MC1R |
| (3) | |
| Oculocutaneous albinism, type IV, 606574 | MATP, AIM1 |
| (3) | |
| Oculodentodigital dysplasia, 164200 (3) | GJA1, CX43, ODDD, SDTY3, ODOD |
| Oculofaciocardiodental syndrome, 300166 | BCOR, KIAA1575, MAA2, ANOP2 |
| (3) | |
| Oculopharyngeal muscular dystorphy, | PABPN1, PABP2, PAB2 |
| 164300 (3) | |
| Oculopharyngeal muscular dystrophy, | PABPN1, PABP2, PAB2 |
| autosomal recessive, 257950 (3) | |
| Odontohypophosphatasia, 146300 (3) | ALPL, HOPS, TNSALP |
| Oguchi disease-1, 258100 (3) | SAG |
| Oguchi disease-2, 258100 (3) | RHOK, RK, GRK1 |
| Oligodendroglioma, 137800 (3) | PTEN, MMAC1 |
| Oligodontia, 604625 (3) | PAX9 |
| Oligodontia-colorectal cancer syndrome, | AXIN2 |
| 608615 (3) | |
| Omenn syndrome, 603554 (3) | DCLRE1C, ARTEMIS, SCIDA |
| Omenn syndrome, 603554 (3) | RAG1 |
| Omenn syndrome, 603554 (3) | RAG2 |
| Opitz G syndrome, type I, 300000 (3) | MID1, OGS1, BBBG1, FXY, OSX |
| Opremazole poor metabolizer (3) | CYP2C, CYP2C19 |
| Optic atrophy 1, 165500 (3) | OPA1, NTG, NPG |
| Optic atrophy and cataract, 165300 (3) | OPA3, MGA3 |
| Optic nerve coloboma with renal disease, | PAX2 |
| 120330 (3) | |
| Optic nerve hypoplasia/aplasia, 165550 (3) | PAX6, AN2, MGDA |
| Oral-facial-digital syndrome 1, 311200 (3) | OFD1, CXorf5 |
| Ornithine transcarbamylase deficiency, | OTC |
| 311250 (3) | |
| Orofacial cleft 6, 608864 (3) | IRF6, VWS, LPS, PIT, PPS, OFC6 |
| Orolaryngeal cancer, multiple, (3) | CDKN2A, MTS1, P16, MLM, CMM2 |
| Oroticaciduria (3) | UMPS, OPRT |
| Orthostatic intolerance, 604715 (3) | SLC6A2, NAT1, NET1 |
| OSMED syndrome, 215150 (3) | COL11A2, STL3, DFNA13 |
| Osseous heteroplasia, progressive, 166350 | GNAS, GNAS1, GPSA, POH, PHP1B, |
| (3) | PHP1A, AHO |
| Ossification of posterior longitudinal | ENPP1, PDNP1, NPPS, M6S1, PCA1 |
| ligament of spine, 602475 (3) | |
| Osteoarthritis, hand, susceptibility to, | MATN3, EDM5, HOA |
| 607850 (3) | |
| Osteoarthritis of hip, female-specific, | FRZB, FRZB1, SRFP3 |
| susceptibility to, 165720 (3) | |
| Osteoarthritis, susceptibility to, 165720 (3) | ASPN, PLAP1 |
| Osteoarthrosis, 165720 (3) | COL2A1 |
| Osteogenesis imperfecta, 3 clinical forms, | COL1A2 |
| 166200, 166210, 259420 (3) | |
| Osteogenesis imperfecta, type I, 166200 (3) | COL1A1 |
| Osteogenesis imperfecta, type II, 166210 | COL1A1 |
| (3) | |
| Osteogenesis imperfecta, type III, 259420 | COL1A1 |
| (3) | |
| Osteogenesis imperfecta, type IV, 166220 | COL1A1 |
| (3) | |
| Osteolysis, familial expansile, 174810 (3) | TNFRSF11A, RANK, ODFR, OFE |
| Osteolysis, idiopathic, Saudi type, 605156 | MMP2, CLG4A, MONA |
| (3) | |
| Osteopetrosis, autosomal dominant, type I, | LRP5, BMND1, LRP7, LR3, OPPG, |
| 607634 (3) | VBCH2 |
| Osteopetrosis, autosomal dominant, type II, | CLCN7, CLC7, OPTA2 |
| 166600 (3) | |
| Osteopetrosis, autosomal recessive, | OSTM1, GL |
| 259700 (3) | |
| Osteopetrosis, recessive, 259700 (3) | CLCN7, CLC7, OPTA2 |
| Osteopetrosis, recessive, 259700 (3) | TCIRG1, TIRC7, OC116, OPTB1 |
| Osteopoikilosis, 166700 (3) | LEMD3, MAN1 |
| Osteoporosis, 166710 (3) | COL1A1 |
| Osteoporosis, 166710 (3) | LRP5, BMND1, LRP7, LR3, OPPG, |
| | VBCH2 |
| Osteoporosis (3) | CALCA, CALC1 |
| Osteoporosis, hypophosphatemic, (3) | SLC17A2, NPT2 |
| Osteoporosis, idiopathic, 166710 (3) | COL1A2 |
| Osteoporosis, postmenopausal, | CALCR, CRT |
| susceptibility, 166710 (3) | |
| Osteoporosis-pseudoglioma syndrome, | LRP5, BMND1, LRP7, LR3, OPPG, |
| 259770 (3) | VBCH2 |
| Osteoporosis, susceptibility to, 166710 (3) | RIL |
| Osteosarcoma (3) | TP53, P53, LFS1 |
| Osteosarcoma, somatic, 259500 (3) | CHEK2, RAD53, CHK2, CDS1, LFS2 |
| Otopalatodigital syndrome, type I, 311300 | FLNA, FLN1, ABPX, NHBP, OPD1, |
| (3) | OPD2, FMD, MNS |
| Otopalatodigital syndrome, type II, 304120 | FLNA, FLN1, ABPX, NHBP, OPD1, |
| (3) | OPD2, FMD, MNS |
| Ovarian cancer (3) | BRCA1, PSCP |
| Ovarian cancer (3) | MSH2, COCA1, FCC1, HNPCC1 |
| Ovarian cancer, 604370 (3) | PIK3CA |
| Ovarian cancer, endometrial type (3) | MSH6, GTBP, HNPCC5 |
| Ovarian cancer, somatic, (3) | ERBB2, NGL, NEU, HER2 |
| Ovarian carcinoma (3) | CDH1, UVO |
| Ovarian carcinoma (3) | RRAS2, TC21 |
| Ovarian carcinoma, endometrioid type (3) | CTNNB1 |
| Ovarian dysgenesis 1, 233300 (3) | FSHR, ODG1 |
| Ovarian dysgenesis 2, 300510 (3) | BMP15, GDF9B, ODG2 |
| Ovarian hyperstimulation syndrome, | FSHR, ODG1 |
| gestational, 608115 (3) | |
| Ovarian sex cord tumors (3) | FSHR, ODG1 |
| Ovarioleukodystrophy, 603896 (3) | EIF2B2 |
| Ovarioleukodystrophy, 603896 (3) | EIF2B4 |
| Ovarioleukodystrophy, 603896 (3) | EIF2B5, LVWM, CACH, CLE |
| Pachyonychia congenita, Jackson-Lawler | KRT17, PC2, PCHC1 |
| type, 167210 (3) | |
| Pachyonychia congenita, Jackson-Lawler | KRT6B, PC2 |
| type, 167210 (3) | |
| Pachyonychia congenita, Jadassohn- | KRT16 |
| Lewandowsky type, 167200 (3) | |
| Pachyonychia congenita, Jadassohn- | KRT6A |
| Lewandowsky type, 167200 (3) | |
| Paget disease, juvenile, 239000 (3) | TNFRSF11B, OPG, OCIF |
| Paget disease of bone, 602080 (3) | SQSTM1, P62, PDB3 |
| Paget disease of bone, 602080 (3) | TNFRSF11A, RANK, ODFR, OFE |
| Pallidopontonigral degeneration, 168610 (3) | MAPT, MTBT1, DDPAC, MSTD |
| Pallister-Hall syndrome, 146510 (3) | GLI3, PAPA, PAPB, ACLS |
| Palmoplantar keratoderma, | KRT16 |
| nonepidermolytic, 600962 (3) | |
| Palmoplantar verrucous nevus, unilateral, | KRT16 |
| 144200 (3) | |
| Pancreatic agenesis, 260370 (3) | IPF1 |
| Pancreatic cancer, 260350 (3) | ARMET, ARP |
| Pancreatic cancer, 260350 (3) | BRCA2, FANCD1 |
| Pancreatic cancer, 260350 (3) | TP53, P53, LFS1 |
| Pancreatic cancer (3) | MADH4, DPC4, SMAD4, JIP |
| Pancreatic cancer/melanoma syndrome, | CDKN2A, MTS1, P16, MLM, CMM2 |
| 606719 (3) | |
| Pancreatic cancer, somatic (3) | ACVR1B, ACVRLK4, ALK4 |
| Pancreatic cancer, sporadic (3) | STK11, PJS, LKB1 |
| Pancreatic carcinoma, somatic, 260350 (3) | KRAS2, RASK2 |
| Pancreatic carcinoma, somatic (3) | RBBP8, RIM |
| Pancreatitis, hereditary, 167800 (3) | PRSS1, TRY1 |
| Pancreatitis, hereditary, 167800 (3) | SPINK1, PSTI, PCTT, TATI |
| Pancreatitis, idiopathic (3) | CFTR, ABCC7, CF, MRP7 |
| Papillary serous carcinoma of the | BRCA1, PSCP |
| peritoneum (3) | |
| Papillon-Lefevre syndrome, 245000 (3) | CTSC, CPPI, PALS, PLS, HMS |
| Paraganglioma, familial malignant, 168000 | SDHB, SDH1, SDHIP |
| (3) | |
| Paragangliomas, familial central nervous | SDHD, PGL1 |
| system, 168000 (3) | |
| Paragangliomas, familial nonchromaffin, 1, | SDHD, PGL1 |
| with and without deafness, 168000 (3) | |
| Paragangliomas, familial nonchromaffin, 3, | SDHC, PGL3 |
| 605373 (3) | |
| Paraganglioma, sporadic corotid body, | SDHD, PGL1 |
| 168000 (3) | |
| Paramyotonia congenita, 168300 (3) | SCN4A, HYPP, NAC1A |
| Parathyroid adenoma, sporadic (3) | MEN1 |
| Parathyroid adenoma with cystic changes, | HRPT2, C1orf28 |
| 145001 (3) | |
| Parathyroid carcinoma, 608266 (3) | HRPT2, C1orf28 |
| Parietal foramina 1, 168500 (3) | MSX2, CRS2, HOX8 |
| Parietal foramina 2, 168500 (3) | ALX4, PFM2, FPP |
| Parietal foramina with cleidocranial | MSX2, CRS2, HOX8 |
| dysplasia, 168550 (3) | |
| Parkes Weber syndrome, 608355 (3) | RASA1, GAP, CMAVM, PKWS |
| Parkinson disease, 168600 (3) | NR4A2, NURR1, NOT, TINUR |
| Parkinson disease, 168600 (3) | SNCAIP |
| Parkinson disease, 168600 (3) | TBP, SCA17 |
| Parkinson disease 4, autosomal dominant | SNCA, NACP, PARK1, PARK4 |
| Lewy body, 605543 (3) | |
| Parkinson disease 7, autosomal recessive | DJ1, PARK7 |
| early-onset, 606324 (3) | |
| Parkinson disease-8, 607060 (3) | LRRK2, PARK8 |
| Parkinson disease, early onset, 605909 (3) | PINK1, PARK6 |
| Parkinson disease, familial, 168600 (3) | UCHL1, PARK5 |
| Parkinson disease, familial, 168601 (3) | SNCA, NACP, PARK1, PARK4 |
| Parkinson disease, juvenile, type 2, 600116 | PRKN, PARK2, PDJ |
| (3) | |
| Parkinson disease, resistance to, 168600 | DBH |
| (3) | |
| Parkinson disease, susceptibility to, 168600 | NDUFV2 |
| (3) | |
| Paroxysmal nocturnal hemoglobinuria (3) | PIGA |
| Paroxysmal nonkinesigenic dyskinesia, | MR1, TAHCCP2, KIPP1184, BRP17, |
| 118800 (3) | PNKD, FPD1, PDC, DYT8 |
| Partington syndrome, 309510 (3) | ARX, ISSX, PRTS, MRXS1, MRX36, |
| | MRX54 |
| PCWH, 609136 (3) | SOX10, WS4 |
| Pelger-Huet anomaly, 169400 (3) | LBR, PHA |
| Pelizaeus-Merzbacher disease, 312080 (3) | PLP1, PMD |
| Pelizaeus-Merzbacher-like disease, | GJA12, CX47, PMLDAR |
| autosomal recessive, 608804 (3) | |
| Pendred syndrome, 274600 (3) | SLC26A4, PDS, DFNB4 |
| Perineal hypospadias (3) | AR, DHTR, TFM, SBMA, KD, SMAX1 |
| Periodic fever, familial, 142680 (3) | TNFRSF1A, TNFR1, TNFAR, FPF |
| Periodontitis, juvenile, 170650 (3) | CTSC, CPPI, PALS, PLS, HMS |
| Periventricular heterotopia with | ARFGEF2, BIG2 |
| microcephaly, 608097 (3) | |
| Peroxisomal biogenesis disorder, | PEX6, PXAAA1, PAF2 |
| complementation group 4 (3) | |
| Peroxisomal biogenesis disorder, | PEX6, PXAAA1, PAF2 |
| complementation group 6 (3) | |
| Peroxisome biogenesis factor 12 (3) | PEX12 |
| Persistent hyperinsulinemic hypoglycemia of | KCNJ11, BIR, PHHI |
| infancy, 256450 (3) | |
| Persistent Mullerian duct syndrome, type I, | AMH, MIF |
| 261550 (3) | |
| Persistent Mullerian duct syndrome, type II, | AMHR2, AMHR |
| 261550 (3) | |
| Peters anomaly, 603807 (3) | PAX6, AN2, MGDA |
| Peters anomaly, 604229 (3) | CYP1B1, GLC3A |
| Peutz-Jeghers syndrome, 175200 (3) | STK11, PJS, LKB1 |
| Pfeiffer syndrome, 101600 (3) | FGFR1, FLT2, KAL2 |
| Pfeiffer syndrome, 101600 (3) | FGFR2, BEK, CFD1, JWS |
| Phenylketonuria (3) | PAH, PKU1 |
| Phenylketonuria due to dihydropteridine | QDPR, DHPR |
| reductase deficiency (3) | |
| Phenylketonuria due to PTS deficiency (3) | PTS |
| Phenylthiocarbamide tasting, 171200 (3) | TAS2R38, T2R61, PTC |
| Pheochromocytoma, 171300 (3) | SDHD, PGL1 |
| Pheochromocytoma, 171300 (3) | VHL |
| Pheochromocytoma, extraadrenal, and | SDHB, SDH1, SDHIP |
| cervical paraganglioma, 115310 (3) | |
| Phosphoglycerate dehydrogenase | PHGDH |
| deficiency, 601815 (3) | |
| Phosphoribosyl pyrophosphate synthetase- | PRPS1 |
| related gout (3) | |
| Phosphorylase kinase deficiency of liver and | PHKB |
| muscle, autosomal recessive, 261750 (3) | |
| Phosphoserine phosphatase deficiency (3) | PSP |
| Pick disease, 172700 (3) | PSEN1, AD3 |
| Piebaldism (3) | KIT, PBT |
| Pigmentation of hair, skin, and eyes, | MATP, AIM1 |
| variation in (3) | |
| Pigmented adrenocortical disease, primary | PRKAR1A, TSE1, CNC1, CAR |
| isolated, 160980 (3) | |
| Pigmented paravenous chorioretinal | CRB1, RP12 |
| atrophy, 172870 (3) | |
| Pilomatricoma, 132600 (3) | CTNNB1 |
| Pituitary ACTH-secreting adenoma (3) | GNAI2, GNAI2B, GIP |
| Pituitary ACTH secreting adenoma (3) | GNAS, GNAS1, GPSA, POH, PHP1B, |
| | PHP1A, AHO |
| Pituitary adenoma, nonfunctioning (3) | THRA, ERBA1, THRA1 |
| Pituitary anomalies with holoprosencephaly- | GLI2 |
| like features (3) | |
| Pituitary hormone deficiency, combined (3) | POU1F1, PIT1 |
| Pituitary hormone deficiency, combined (3) | PROP1 |
| Pituitary hormone deficiency, combined, | HESX1, RPX |
| HESX1-related, 182230 (3) | |
| Pituitary hormone deficiency, combined, | LHX3 |
| with rigid cervical spine, 262600 (3) | |
| Pituitary tumor, invasive (3) | PRKCA, PKCA |
| Placental abruption (3) | NOS3 |
| Placental steroid sulfatase deficiency (3) | STS, ARSC1, ARSC, SSDD |
| Plasmin inhibitor deficiency (3) | PLI, SERPINF2 |
| Plasminogen Tochigi disease (3) | PLG |
| Platelet-activating factor acetylhydrolase | PLA2G7, PAFAH |
| deficiency (3) | |
| Platelet ADP receptor defect (3) | P2RY12, P2Y12 |
| Platelet disorder, familial, with associated | RUNX1, CBFA2, AML1 |
| myeloid malignancy, 601399 (3) | |
| Platelet glycoprotein IV deficiency, 608404 | CD36 |
| (3) | |
| Pneumonitis, desquamative interstitial, | SFTPC, SFTP2 |
| 263000 (3) | |
| Pneumothorax, primary spontaneous, | FLCN, BHD |
| 173600 (3) | |
| Polycystic kidney and hepatic disease, | FCYT, PKHD1, ARPKD |
| 263200 (3) | |
| Polycystic kidney disease, adult type I, | PKD1 |
| 173900 (3) | |
| Polycystic kidney disease, adult, type II (3) | PKD2, PKD4 |
| Polycystic kidney disease, infantile severe, | PKDTS |
| with tuberous sclerosis (3) | |
| Polycystic liver disease, 174050 (3) | PRKCSH, G19P1, PCLD |
| Polycystic liver disease, 174050 (3) | SEC63 |
| Polycythemia, benign familial, 263400 (3) | VHL |
| Polycythemia vera, 263300 (3) | JAK2 |
| Polydactyly, postaxial, types A1 and B, | GLI3, PAPA, PAPB, ACLS |
| 174200 (3) | |
| Polydactyly, preaxial, type IV, 174700 (3) | GLI3, PAPA, PAPB, ACLS |
| Polymicrogyria, bilateral frontoparietal, | GPR56, TM7XN1, BFPP |
| 606854 (3) | |
| Polyposis, juvenile intestinal, 174900 (3) | BMPR1A, ACVRLK3, ALK3 |
| Polyposis, juvenile intestinal, 174900 (3) | MADH4, DPC4, SMAD4, JIP |
| Popliteal pterygium syndrome, 119500 (3) | IRF6, VWS, LPS, PIT, PPS, OFC6 |
| Porencephaly, 175780 (3) | COL4A1 |
| Porphyria, acute hepatic (3) | ALAD |
| Porphyria, acute intermittent (3) | HMBS, PBGD, UPS |
| Porphyria, acute intermittent, nonerythroid | HMBS, PBGD, UPS |
| variant (3) | |
| Porphyria, congenital erythropoietic, 263700 | UROS |
| (3) | |
| Porphyria cutanea tarda (3) | UROD |
| Porphyria, hepatoerythropoietic (3) | UROD |
| Porphyria variegata, 176200 (3) | HFE, HLA-H, HFE1 |
| Porphyria variegata, 176200 (3) | PPOX |
| PPM-X syndrome, 300055 (3) | MECP2, RTT, PPMX, MRX16, MRX79 |
| Prader-Willi syndrome, 176270 (3) | NDN |
| Prader-Willi syndrome, 176270 (3) | SNRPN |
| Precocious puberty, male, 176410 (3) | LHCGR |
| Preeclampsia/eclampsia 4 (3) | STOX1, PEE4 |
| Preeclampsia, susceptibility to, 189800 (3) | EPHX1 |
| Preeclampsia, susceptibility to (3) | AGT, SERPINA8 |
| Prekallikrein deficiency (3) | KLKB1, KLK3 |
| Premature chromosome condensation with | MCPH1 |
| microcephaly and mental retardation, | |
| 606858 (3) | |
| Premature ovarian failure, 300511 (3) | DIAPH2, DIA, POF2 |
| Premature ovarian failure 3, 608996 (3) | FOXL2, BPES, BPES1, PFRK, POF3 |
| Primary lateral sclerosis, juvenile, 606353 | ALS2, ALSJ, PLSJ, IAHSP |
| (3) | |
| Prion disease with protracted course, | PRNP, PRIP |
| 606688 (3) | |
| Progressive external ophthalmoplegia with | C10orf2, TWINKLE, PEO1, PEO |
| mitochondrial DNA deletions, 157640 (3) | |
| Progressive external ophthalmoplegia with | POLG, POLG1, POLGA, PEO |
| mitochondrial DNA deletions, 157640 (3) | |
| Progressive external ophthalmoplegia with | SLC25A4, ANT1, T1, PEO3 |
| mitochondrial DNA deletions, 157640 (3) | |
| Proguanil poor metabolizer (3) | CYP2C, CYP2C19 |
| Prolactinoma, hyperparathyroidism, | MEN1 |
| carcinoid syndrome (3) | |
| Prolidase deficiency (3) | PEPD |
| Properdin deficiency, X-linked, 312060 (3) | PFC, PFD |
| Propionicacidemia, 606054 (3) | PCCA |
| Propionicacidemia, 606054 (3) | PCCB |
| Prostate cancer 1, 176807, 601518 (3) | RNASEL, RNS4, PRCA1, HPC1 |
| Prostate cancer, 176807 (3) | BRCA2, FANCD1 |
| Prostate cancer, 176807 (3) | PTEN, MMAC1 |
| Prostate cancer (3) | AR, DHTR, TFM, SBMA, KD, SMAX1 |
| Prostate cancer, familial, 176807 (3) | CHEK2, RAD53, CHK2, CDS1, LFS2 |
| Prostate cancer, hereditary, 176807 (3) | MSR1 |
| Prostate cancer, progression and | EPHB2, EPHT3, DRT, ERK |
| metastasis of, 176807 (3) | |
| Prostate cancer, somatic, 176807 (3) | KLF6, COPEB, BCD1, ZF9 |
| Prostate cancer, somatic, 176807 (3) | MAD1L1, TXBP181 |
| Prostate cancer, susceptibility to, 176807 | AR, DHTR, TFM, SBMA, KD, SMAX1 |
| (3) | |
| Prostate cancer, susceptibility to, 176807 | ATBF1 |
| (3) | |
| Prostate cancer, susceptibility to, 176807 | ELAC2, HPC2 |
| (3) | |
| Prostate cancer, susceptibility to, 176807 | MXI1 |
| (3) | |
| Protein S deficiency (3) | PROS1 |
| Proteinuria, low molecular weight, with | CLCN5, CLCK2, NPHL2, DENTS |
| hypercalciuric nephrocalcinosis (3) | |
| Protoporphyria, erythropoietic (3) | FECH, FCE |
| Protoporphyria, erythropoietic, recessive, | FECH, FCE |
| with liver failure (3) | |
| Proud syndrome, 300004 (3) | ARX, ISSX, PRTS, MRXS1, MRX36, |
| | MRX54 |
| Pseudoachondroplasia, 177170 (3) | COMP, EDM1, MED, PSACH |
| Pseudohermaphroditism, male, with | HSD17B3, EDH17B3 |
| gynecomastia, 264300 (3) | |
| Pseudohermaphroditism, male, with Leydig | LHCGR |
| cell hypoplasia (3) | |
| Pseudohypoaldosteronism, type I, 264350 | SCNN1A |
| (3) | |
| Pseudohypoaldosteronism, type I, 264350 | SCNN1B |
| (3) | |
| Pseudohypoaldosteronism, type I, 264350 | SCNN1G, PHA1 |
| (3) | |
| Pseudohypoaldosteronism type I, autosomal | NR3C2, MLR, MCR |
| dominant, 177735 (3) | |
| Pseudohypoaldosteronism type II (3) | WNK4, PRKWNK4, PHA2B |
| Pseudohypoaldosteronism, type IIC, 145260 | WNK1, PRKWNK1, KDP, PHA2C |
| (3) | |
| Pseudohypoparathyroidism, type Ia, 103580 | GNAS, GNAS1, GPSA, POH, PHP1B, |
| (3) | PHP1A, AHO |
| Pseudohypoparathyroidism, type Ib, 603233 | GNAS, GNAS1, GPSA, POH, PHP1B, |
| (3) | PHP1A, AHO |
| Pseudovaginal perineoscrotal hypospadias, | SRD5A2 |
| 264600 (3) | |
| Pseudovitamin D deficiency rickets 1 (3) | CYP27B1, PDDR, VDD1 |
| Pseudoxanthoma elasticum, autosomal | ABCC6, ARA, ABC34, MLP1, PXE |
| dominant, 177850 (3) | |
| Pseudoxanthoma elasticum, autosomal | ABCC6, ARA, ABC34, MLP1, PXE |
| recessive, 264800 (3) | |
| Psoriasis, susceptibility to, 177900 (3) | PSORS6 |
| Psoriatic arthritis, susceptibility to, 607507 | CARD15, NOD2, IBD1, CD, ACUG, |
| (3) | PSORAS1 |
| Pulmonary alveolar proteinosis, 265120 (3) | CSF2RB |
| Pulmonary alveolar proteinosis, 265120 (3) | SFTPC, SFTP2 |
| Pulmonary alveolar proteinosis, congenital, | SFTPB, SFTB3 |
| 265120 (3) | |
| Pulmonary fibrosis, idiopathic, familial, | SFTPC, SFTP2 |
| 178500 (3) | |
| Pulmonary fibrosis, idiopathic, susceptibility | SFTPA1, SFTP1 |
| to, 178500 (3) | |
| Pulmonary hypertension, familial primary, | BMPR2, PPH1 |
| 178600 (3) | |
| Pycnodysostosis, 265800 (3) | CTSK |
| Pyloric stenosis, infantile hypertrophic, | NOS1 |
| susceptibility to, 179010 (3) | |
| Pyogenic sterile arthritis, pyoderma | PSTPIP1, PSTPIP, CD2BP1, PAPAS |
| gangrenosum, and acne, 604416 (3) | |
| Pyropoikilocytosis (3) | SPTA1 |
| Pyruvate carboxylase deficiency, 266150 (3) | PC |
| Pyruvate dehydrogenase deficiency (3) | PDHA1, PHE1A |
| Pyruvate dehydrogenase E1-beta deficiency | PDHB |
| (3) | |
| Rabson-Mendenhall syndrome, 262190 (3) | INSR |
| Radioulnar synostosis with amegakaryocytic | HOXA11, HOX1I |
| thrombocytopenia, 605432 (3) | |
| RAPADILINO syndrome, 266280 (3) | RECQL4, RTS, RECQ4 |
| Rapid progression to AIDS from HIV1 | CX3CR1, GPR13, V28 |
| infection (3) | |
| Rapp-Hodgkin syndrome, 129400 (3) | TP73L, TP63, KET, EEC3, SHFM4, |
| | LMS, RHS |
| Red hair/fair skin (3) | MC1R |
| Refsum disease, 266500 (3) | PEX7, RCDP1 |
| Refsum disease, 266500 (3) | PHYH, PAHX |
| Refsum disease, infantile, 266510 (3) | PEX1, ZWS1 |
| Refsum disease, infantile form, 266510 (3) | PEX26 |
| Refsum disease, infantile form, 266510 (3) | PXMP3, PAF1, PMP35, PEX2 |
| Renal carcinoma, chromophobe, somatic, | FLCN, BHD |
| 144700 (3) | |
| Renal cell carcinoma, 144700 (3) | TRC8, RCA1, HRCA1 |
| Renal cell carcinoma, clear cell, somatic, | OGG1 |
| 144700 (3) | |
| Renal cell carcinoma, papillary, 1, 605074 | PRCC, RCCP1 |
| (3) | |
| Renal cell carcinoma, papillary, 1, 605074 | TFE3 |
| (3) | |
| Renal cell carcinoma, papillary, familial and | MET |
| sporadic, 605074 (3) | |
| Renal cell carcinoma, somatic (3) | VHL |
| Renal glucosuria, 233100 (3) | SLC5A2, SGLT2 |
| Renal hypoplasia, isolated (3) | PAX2 |
| Renal tubular acidosis, distal, 179800, | SLC4A1, AE1, EPB3 |
| 602722 (3) | |
| Renal tubular acidosis, distal, autosomal | ATP6V0A4, ATP6N1B, VPP2, RTA1C, |
| recessive, 602722 (3) | RTADR |
| Renal tubular acidosis-osteopetrosis | CA2 |
| syndrome (3) | |
| Renal tubular acidosis, proximal, with ocular | SLC4A4, NBC1, KNBC, SLC4A5 |
| abnormalities, 604278 (3) | |
| Renal tubular acidosis with deafness, | ATP6B1, VPP3 |
| 267300 (3) | |
| Renal tubular dysgenesis, 267430 (3) | ACE, DCP1, ACE1 |
| Renal tubular dysgenesis, 267430 (3) | AGTR1, AGTR1A, AT2R1 |
| Renal tubular dysgenesis, 267430 (3) | AGT, SERPINA8 |
| Renal tubular dysgenesis, 267430 (3) | REN |
| Renpenning syndrome, 309500 (3) | PQBP1, NPW38, SHS, MRX55, |
| | MRXS3, RENS1, MRXS8 |
| Response to morphine-6-glucuronide (3) | OPRM1 |
| Resting heart rate, 607276 (3) | ADRB1, ADRB1R, RHR |
| Restrictive dermopathy, lethal, 275210 (3) | ZMPSTE24, FACE1, STE24, MADB |
| Retinal degeneration, autosomal recessive, | NRL, D14S46E, RP27 |
| clumped pigment type (3) | |
| Retinal degeneration, autosomal recessive, | PROM1, PROML1, AC133 |
| prominin-related (3) | |
| Retinal degeneration, late-onset, autosomal | C1QTNF5, CTRP5, LORD |
| dominant, 605670 (3) | |
| Retinal dystrophy, early-onset severe (3) | LRAT |
| Retinitis pigmentosa-10, 180105 (3) | IMPDH1 |
| Retinitis pigmentosa-11, 600138 (3) | PRPF31, PRP31 |
| Retinitis pigmentosa-1, 180100 (3) | RP1, ORP1 |
| Retinitis pigmentosa-12, autosomal | CRB1, RP12 |
| recessive, 600105 (3) | |
| Retinitis pigmentosa-13, 600059 (3) | PRPF8, PRPC8, RP13 |
| Retinitis pigmentosa-14, 600132 (3) | TULP1, RP14 |
| Retinitis pigmentosa-17, 600852 (3) | CA4, RP17 |
| Retinitis pigmentosa-18, 601414 (3) | HPRP3, RP18 |
| Retinitis pigmentosa-19, 601718 (3) | ABCA4, ABCR, STGD1, FFM, RP19 |
| Retinitis pigmentosa-20 (3) | RPE65, RP20 |
| Retinitis pigmentosa-2 (3) | RP2 |
| Retinitis pigmentosa-26, 608380 (3) | CERKL |
| Retinitis pigmentosa-27 (3) | NRL, D14S46E, RP27 |
| Retinitis pigmentosa-30, 607921 (3) | FSCN2, RFSN |
| Retinitis pigmentosa-3, 300389 (3) | RPGR, RP3, CRD, RP15, COD1 |
| Retinitis pigmentosa-4, autosomal dominant | RHO, RP4, OPN2 |
| (3) | |
| Retinitis pigmentosa-7, 608133 (3) | RDS, RP7, PRPH2, PRPH, AVMD, |
| | AOFMD |
| Retinitis pigmentosa-9, 180104 (3) | RP9 |
| Retinitis pigmentosa, AR, 268000 (3) | RLBP1 |
| Retinitis pigmentosa, AR, without hearing | USH2A |
| loss, 268000 (3) | |
| Retinitis pigmentosa, autosomal dominant | RGR |
| (3) | |
| Retinitis pigmentosa, autosomal recessive, | CNGB1, CNCG3L, CNCG2 |
| 268000 (3) | |
| Retinitis pigmentosa, autosomal recessive | CNGA1, CNCG1 |
| (3) | |
| Retinitis pigmentosa, autosomal recessive | PDE6A, PDEA |
| (3) | |
| Retinitis pigmentosa, autosomal recessive | PDE6B, PDEB, CSNB3 |
| (3) | |
| Retinitis pigmentosa, autosomal recessive | RGR |
| (3) | |
| Retinitis pigmentosa, autosomal recessive | RHO, RP4, OPN2 |
| (3) | |
| Retinitis pigmentosa, digenic (3) | ROM1, ROSP1 |
| Retinitis pigmentosa, digenic, 608133 (3) | RDS, RP7, PRPH2, PRPH, AVMD, |
| | AOFMD |
| Retinitis pigmentosa, juvenile (3) | AIPL1, LCA4 |
| Retinitis pigmentosa, late onset, 268000 (3) | NR2E3, PNR, ESCS |
| Retinitis pigmentosa, late-onset dominant, | CRX, CORD2, CRD |
| 268000 (3) | |
| Retinitis pigmentosa, MERTK-related, | MERTK |
| 268000 (3) | |
| Retinitis pigmentosa, X-linked with deafness | RPGR, RP3, CRD, RP15, COD1 |
| and sinorespiratory infections, 300455 (3) | |
| Retinitis pigmentosa, X-linked, with | RPGR, RP3, CRD, RP15, COD1 |
| recurrent respiratory infections, 300455 (3) | |
| Retinitis punctata albescens, 136880 (3) | RDS, RP7, PRPH2, PRPH, AVMD, |
| | AOFMD |
| Retinitis punctata albescens, 136880 (3) | RLBP1 |
| Retinoblastoma (3) | RB1 |
| Retinol binding protein, deficiency of (3) | RBP4 |
| Retinoschisis (3) | RS1, XLRS1 |
| Rett syndrome, 312750 (3) | MECP2, RTT, PPMX, MRX16, MRX79 |
| Rett syndrome, atypical, 312750 (3) | CDKL5, STK9 |
| Rett syndrome, preserved speech variant, | MECP2, RTT, PPMX, MRX16, MRX79 |
| 312750 (3) | |
| Rhabdoid predisposition syndrome, familial | SMARCB1, SNF5, INI1, RDT |
| (3) | |
| Rhabdoid tumors (3) | SMARCB1, SNF5, INI1, RDT |
| Rhabdomyosarcoma, 268210 (3) | SLC22A1L, BWSCR1A, IMPT1 |
| Rhabdomyosarcoma, alveolar, 268220 (3) | FOXO1A, FKHR |
| Rhabdomyosarcoma, alveolar, 268220 (3) | PAX3, WS1, HUP2, CDHS |
| Rhabdomyosarcoma, alveolar, 268220 (3) | PAX7 |
| Rheumatoid arthritis, progression of, | IL10, CSIF |
| 180300 (3) | |
| Rheumatoid arthritis, susceptibility to, | MHC2TA, C2TA |
| 180300 (3) | |
| Rheumatoid arthritis, susceptibility to, | NFKBIL1 |
| 180300 (3) | |
| Rheumatoid arthritis, susceptibility to, | PADI4, PADI5, PAD |
| 180300 (3) | |
| Rheumatoid arthritis, susceptibility to, | PTPN8, PEP, PTPN22, LYP |
| 180300 (3) | |
| Rheumatoid arthritis, susceptibility to, | RUNX1, CBFA2, AML1 |
| 180300 (3) | |
| Rheumatoid arthritis, susceptibility to, | SLC22A4, OCTN1 |
| 180300 (3) | |
| Rheumatoid arthritis, systemic juvenile, | MIF |
| susceptibility to, 604302 (3) | |
| Rhizomelic chondrodysplasia punctata, type | PEX7, RCDP1 |
| 1, 215100 (3) | |
| Rhizomelic chondrodysplasia punctata, type | AGPS, ADHAPS |
| 3, 600121 (3) | |
| Rh-mod syndrome (3) | RHAG, RH50A |
| Rh-negative blood type (3) | RHD |
| Rh-null disease, amorph type (3) | RHCE |
| Ribose 5-phosphate isomerase deficiency, | RPIA, RPI |
| 608611 (3) | |
| Rickets due to defect in vitamin D 25- | CYP2R1 |
| hydroxylation, 600081 (3) | |
| Rickets, vitamin D-resistant, type IIA, | VDR |
| 277440 (3) | |
| Rickets, vitamin D-resistant, type IIB, | VDR |
| 277420 (3) | |
| Rieger anomaly (3) | FOXC1, FKHL7, FREAC3 |
| Rieger syndrome, 180500 (3) | PITX2, IDG2, RIEG1, RGS, IGDS2 |
| Ring dermoid of cornea, 180550 (3) | PITX2, IDG2, RIEG1, RGS, IGDS2 |
| Rippling muscle disease, 606072 (3) | CAV3, LGMD1C |
| Roberts syndrome, 268300 (3) | ESCO2 |
| Robinow syndrome, autosomal recessive, | ROR2, BDB1, BDB, NTRKR2 |
| 268310 (3) | |
| Rokitansky-Kuster-Hauser syndrome, | WNT4 |
| 277000 (3) | |
| Rothmund-Thomson syndrome, 268400 (3) | RECQL4, RTS, RECQ4 |
| Roussy-Levy syndrome, 180800 (3) | MPZ, CMT1B, CMTDI3, CHM, DSS |
| Roussy-Levy syndrome, 180800 (3) | PMP22, CMT1A, CMT1E, DSS |
| Rubenstein-Taybi syndrome, 180849 (3) | CREBBP, CBP, RSTS |
| Rubinstein-Taybi syndrome, 180849 (3) | EP300 |
| Saethre-Chotzen syndrome, 101400 (3) | FGFR2, BEK, CFD1, JWS |
| Saethre-Chotzen syndrome, 101400 (3) | TWIST, ACS3, SCS |
| Saethre-Chotzen syndrome with eyelid | TWIST, ACS3, SCS |
| anomalies, 101400 (3) | |
| Salivary adenoma (3) | HMGA2, HMGIC, BABL, LIPO |
| Salla disease, 604369 (3) | SLC17A5, SIASD, SLD |
| Sandhoff disease, infantile, juvenile, and | HEXB |
| adult forms, 268800 (3) | |
| Sanfilippo syndrome, type A, 252900 (3) | SGSH, MPS3A, SFMD |
| Sanfilippo syndrome, type B (3) | NAGLU |
| Sarcoidosis, early-onset, 181000 (3) | CARD15, NOD2, IBD1, CD, ACUG, |
| | PSORAS1 |
| Sarcoidosis, susceptibility to, 181000 (3) | BTNL2 |
| Sarcoidosis, susceptibility to, 181000 (3) | HLA-DR1B |
| Sarcoma, synovial (3) | SSX1, SSRC |
| Sarcoma, synovial (3) | SSX2 |
| SARS, progression of (3) | ACE, DCP1, ACE1 |
| Schimke immunoosseous dysplasia, | SMARCAL1, HARP, SIOD |
| 242900 (3) | |
| Schindler disease, type I, 609241 (3) | NAGA |
| Schindler disease, type III, 609241 (3) | NAGA |
| Schizencephaly, 269160 (3) | EMX2 |
| Schizoaffective disorder, susceptibility to, | DISC1 |
| 181500 (3) | |
| Schizophrenia 5, 603175 (3) | TRAR4 |
| Schizophrenia, chronic (3) | APP, AAA, CVAP, AD1 |
| Schizophrenia, susceptibility to, 181500 (3) | COMT |
| Schizophrenia, susceptibility to, 181500 (3) | DISC1 |
| Schizophrenia, susceptibility to, 181500 (3) | HTR2A |
| Schizophrenia, susceptibility to, 181500 (3) | RTN4R, NOGOR |
| Schizophrenia, susceptibility to, 181500 (3) | SYN2 |
| Schizophrenia, susceptibility to, 181510 (3) | EPN4, EPNR, KIAA0171, SCZD1 |
| Schizophrenia, susceptibility to, 4 600850 | PRODH, PRODH2, SCZD4 |
| (3) | |
| Schwannomatosis, 162091 (3) | NF2 |
| Schwartz-Jampel syndrome, type 1, 255800 | HSPG2, PLC, SJS, SJA, SJS1 |
| (3) | |
| SCID, autosomal recessive, T-negative/B- | JAK3, JAKL |
| positive type (3) | |
| Sclerosteosis, 269500 (3) | SOST |
| Scurvy (3) | GULOP, GULO |
| Sea-blue histiocyte disease, 269600 (3) | APOE, AD2 |
| Seasonal affective disorder, susceptibility to, | HTR2A |
| 608516 (3) | |
| Sebastian syndrome, 605249 (3) | MYH9, MHA, FTNS, DFNA17 |
| Seckel syndrome 1, 210600 (3) | ATR, FRP1, SCKL |
| Segawa syndrome, recessive (3) | TH, TYH |
| Seizures, afebrile, 604233 (3) | SCN2A1, SCN2A |
| Seizures, benign familial neonatal-infantile, | SCN2A1, SCN2A |
| 607745 (3) | |
| Selective T-cell defect (3) | ZAP70, SRK, STD |
| Self-healing collodion baby, 242300 (3) | TGM1, ICR2, LI1 |
| SEMD, Pakistani type (3) | PAPSS2, ATPSK2 |
| Senior-Loken syndrome-1, 266900 (3) | NPHP1, NPH1, SLSN1 |
| Senior-Loken syndrome 4, 606996 (3) | NPHP4, SLSN4 |
| Senior-Loken syndrome 5, 609254 (3) | IQCB1, NPHP5, KIAA0036 |
| Sensory ataxic neuropathy, dysarthria, and | POLG, POLG1, POLGA, PEO |
| ophthalmoparesis, 157640 (3) | |
| Sepiapterin reductase deficiency (3) | SPR |
| Sepsis, susceptibility to (3) | CASP12, CASP12P1 |
| Septic shock, susceptibility to (3) | TNF, TNFA |
| Septooptic dysplasia, 182230 (3) | HESX1, RPX |
| Sertoli cell-only syndrome, susceptibility to, | USP26 |
| 305700 (3) | |
| Severe combined immunodeficiency, | DCLRE1C, ARTEMIS, SCIDA |
| Athabascan type, 602450 (3) | |
| Severe combined immunodeficiency, B cell- | RAG1 |
| negative, 601457 (3) | |
| Severe combined immunodeficiency, B cell- | RAG2 |
| negative, 601457 (3) | |
| Severe combined immunodeficiency due to | ADA |
| ADA deficiency, 102700 (3) | |
| Severe combined immunodeficiency due to | PTPRC, CD45, LCA |
| PTPRC deficiency (3) | |
| Severe combined immunodeficiency, T-cell | IL7R |
| negative, B-cell/natural killer cell-positive | |
| type, 600802 (3) | |
| Severe combined immunodeficiency, T- | CD3D, T3D |
| negative/B-positive type, 600802 (3) | |
| Severe combined immunodeficiency, X- | IL2RG, SCIDX1, SCIDX, IMD4 |
| linked, 300400 (3) | |
| Sex reversal, XY, with adrenal failure (3) | FTZF1, FTZ1, SF1 |
| Sezary syndrome (3) | BCL10 |
| Shah-Waardenburg syndrome, 277580 (3) | EDN3 |
| Short stature, autosomal dominant, with | GHR |
| normal serum growth hormone binding | |
| protein (3) | |
| Short stature, idiopathic (3) | GHR |
| Short stature, idiopathic familial, 604271 (3) | SHOX, GCFX, SS, PHOG |
| Short stature, idiopathic familial, 604271 (3) | SHOXY |
| Short stature, pituitary and cerebellar | LHX4 |
| defects, and small sella turcica, 606606 (3) | |
| Shprintzen-Goldberg syndrome, 182212 (3) | FBN1, MFS1, WMS |
| Shwachman-Diamond syndrome, 260400 | SBDS, SDS |
| (3) | |
| Sialic acid storage disorder, infantile, | SLC17A5, SIASD, SLD |
| 269920 (3) | |
| Sialidosis, type I, 256550 (3) | NEU1, NEU, SIAL1 |
| Sialidosis, type II, 256550 (3) | NEU1, NEU, SIAL1 |
| Sialuria, 269921 (3) | GNE, GLCNE, IBM2, DMRV, NM |
| Sickle cell anemia (3) | HBB |
| Sick sinus syndrome, 608567 (3) | SCN5A, LQT3, IVF, HB1, SSS1 |
| Silver spastic paraplegia syndrome, 270685 | BSCL2, SPG17 |
| (3) | |
| Simpson-Golabi-Behmel syndrome, type 1, | GPC3, SDYS, SGBS1 |
| 312870 (3) | |
| Sitosterolemia, 210250 (3) | ABCG5 |
| Sitosterolemia, 210250 (3) | ABCG8 |
| Situs ambiguus (3) | NODAL |
| Situs inversus viscerum, 270100 (3) | DNAH11, DNAHC11 |
| Sjogren-Larsson syndrome, 270200 (3) | ALDH3A2, ALDH10, SLS, FALDH |
| Skin fragility-woolly hair syndrome, 607655 | DSP, KPPS2, PPKS2 |
| (3) | |
| Slow acetylation (3) | NAT2, AAC2 |
| Slowed nerve conduction velocity, AD, | ARHGEF10, KIAA0294 |
| 608236 (3) | |
| Small patella syndrome, 147891 (3) | TBX4 |
| SMED Strudwick type, 184250 (3) | COL2A1 |
| Smith-Fineman-Myers syndrome, 309580 | ATRX, XH2, XNP, MRXS3, SHS |
| (3) | |
| Smith-Lemli-Opitz syndrome, 270400 (3) | DHCR7, SLOS |
| Smith-Magenis syndrome, 182290 (3) | RAI1, SMCR, SMS |
| Smith-McCort dysplasia, 607326 (3) | DYM, FLJ90130, DMC, SMC |
| Solitary median maxillary central incisor, | SHH, HPE3, HLP3, SMMCI |
| 147250 (3) | |
| Somatotrophinoma (3) | GNAS, GNAS1, GPSA, POH, PHP1B, |
| | PHP1A, AHO |
| Sorsby fundus dystrophy, 136900 (3) | TIMP3, SFD |
| Sotos syndrome, 117550 (3) | NSD1, ARA267, STO |
| Spastic ataxia, Charlevoix-Saguenay type, | SACS, ARSACS |
| 270550 (3) | |
| Spastic paralysis, infantile onset ascending, | ALS2, ALSJ, PLSJ, IAHSP |
| 607225 (3) | |
| Spastic paraplegia 10, 604187 (3) | KIF5A, NKHC, SPG10 |
| Spastic paraplegia-13, 605280 (3) | HSPD1, SPG13, HSP60 |
| Spastic paraplegia-2, 312920 (3) | PLP1, PMD |
| Spastic paraplegia-3A, 182600 (3) | SPG3A |
| Spastic paraplegia-4, 182601 (3) | SPG4, SPAST |
| Spastic paraplegia-6, 600363 (3) | NIPA1, SPG6 |
| Spastic paraplegia-7, 607259 (3) | PGN, SPG7, CMAR, CAR |
| Specific granule deficiency, 245480 (3) | CEBPE, CRP1 |
| Speech-language disorder-1, 602081 (3) | FOXP2, SPCH1, TNRC10, CAGH44 |
| Spermatogenic failure, susceptibility to (3) | DAZL, DAZH, SPGYLA |
| Spherocytosis-1 (3) | SPTB |
| Spherocytosis-2 (3) | ANK1, SPH2 |
| Spherocytosis, hereditary (3) | SLC4A1, AE1, EPB3 |
| Spherocytosis, hereditary, Japanese type | EPB42 |
| (3) | |
| Spherocytosis, recessive (3) | SPTA1 |
| Spina bifida, 601634 (3) | MTHFD, MTHFC |
| Spina bifida, risk of, 601634, 182940 (3) | MTR |
| Spina bifida, risk of, 601634, 182940 (3) | MTRR |
| Spinal and bulbar muscular atrophy of | AR, DHTR, TFM, SBMA, KD, SMAX1 |
| Kennedy, 313200 (3) | |
| Spinal muscrular atrophy, late-onset, Finkel | VAPB, VAPC, ALS8 |
| type, 182980 (3) | |
| Spinal muscular atrophy-1, 253300 (3) | SMN1, SMA1, SMA2, SMA3, SMA4 |
| Spinal muscular atrophy-2, 253550 (3) | SMN1, SMA1, SMA2, SMA3, SMA4 |
| Spinal muscular atrophy-3, 253400 (3) | SMN1, SMA1, SMA2, SMA3, SMA4 |
| Spinal muscular atrophy-4, 271150 (3) | SMN1, SMA1, SMA2, SMA3, SMA4 |
| Spinal muscular atrophy, distal, type V, | BSCL2, SPG17 |
| 600794 (3) | |
| Spinal muscular atrophy, distal, type V, | GARS, SMAD1, CMT2D |
| 600794 (3) | |
| Spinal muscular atrophy, juvenile (3) | HEXB |
| Spinal muscular atrophy with respiratory | IGHMBP2, SMUBP2, CATF1, SMARD1 |
| distress, 604320 (3) | |
| Spinocerebellar ataxia-10 (3) | ATXN10, SCA10 |
| Spinocerebellar ataxia-1, 164400 (3) | ATXN1, ATX1, SCA1 |
| Spinocerebellar ataxia 12, 604326 (3) | PPP2R2B |
| Spinocerebellar ataxia 14, 605361 (3) | PRKCG, PKCC, PKCG, SCA14 |
| Spinocerebellar ataxia 17, 607136 (3) | TBP, SCA17 |
| Spinocerebellar ataxia-2, 183090 (3) | ATXN2, ATX2, SCA2 |
| Spinocerebellar ataxia 25 (3) | SCA25 |
| Spinocerebellar ataxia-27, 609307 (3) | FGF14, FHF4, SCA27 |
| Spinocerebellar ataxia 4, pure Japanese | PLEKHG4 |
| type, 117210 (3) | |
| Spinocerebellar ataxia-6, 183086 (3) | CACNA1A, CACNL1A4, SCA6 |
| Spinocerebellar ataxia-7, 164500 (3) | ATXN7, SCA7, OPCA3 |
| Spinocerebellar ataxia 8, 608768 (3) | SCA8 |
| Spinocerebellar ataxia, autosomal recessive | TDP1 |
| with axonal neuropathy, 607250 (3) | |
| Split hand/foot malformation, type 3, 600095 | SHFM3, DAC |
| (3) | |
| Split-hand/foot malformation, type 4, 605289 | TP73L, TP63, KET, EEC3, SHFM4, |
| (3) | LMS, RHS |
| Spondylocarpotarsal synostosis syndrome, | FLNB, SCT, AOI |
| 272460 (3) | |
| Spondylocostal dysostosis, autosomal | DLL3, SCDO1 |
| recessive, 1, 277300 (3) | |
| Spondylocostal dysostosis, autosomal | MESP2 |
| recessive 2, 608681 (3) | |
| Spondyloepimetaphyseal dysplasia, 608728 | MATN3, EDM5, HOA |
| (3) | |
| Spondyloepiphyseal dysplasia, Kimberley | AGC1, CSPG1, MSK16, SEDK |
| type, 608361 (3) | |
| Spondyloepiphyseal dysplasia, Omani type, | CHST3, C6ST, C6ST1 |
| 608637 (3) | |
| Spondyloepiphyseal dysplasia tarda, | SEDL, SEDT |
| 313400 (3) | |
| Spondyloepiphyseal dysplasia tarda with | WISP3, PPAC, PPD |
| progressive arthropathy, 208230 (3) | |
| Spondylometaphyseal dysplasia, Japanese | COL10A1 |
| type (3) | |
| Squamous cell carcinoma, burn scar- | TNFRSF6, APT1, FAS, CD95, ALPS1A |
| related, somatic (3) | |
| Squamous cell carcinoma, head and neck, | ING1 |
| 601400 (3) | |
| Squamous cell carcinoma, head and neck, | TNFRSF10B, DR5, TRAILR2 |
| 601400 (3) | |
| Stapes ankylosis syndrome without | NOG, SYM1, SYNS1 |
| symphalangism, 184460 (3) | |
| Stargardt disease-1, 248200 (3) | ABCA4, ABCR, STGD1, FFM, RP19 |
| Stargardt disease 3, 600110 (3) | ELOVL4, ADMD, STGD2, STGD3 |
| Startle disease, autosomal recessive (3) | GLRA1, STHE |
| Startle disease/hyperekplexia, autosomal | GLRA1, STHE |
| dominant, 149400 (3) | |
| STAT1 deficiency, complete (3) | STAT1 |
| Statins, attenuated cholesterol lowering by | HMGCR |
| (3) | |
| Steatocystoma multiplex, 184500 (3) | KRT17, PC2, PCHC1 |
| Stem-cell leukemia/lymphoma syndrome (3) | ZNF198, SCLL, RAMP, FIM |
| Stevens-Johnson syndrome, | HLA-B |
| carbamazepine-induced, susceptibility to, | |
| 608579 (3) | |
| Stickler syndrome, type I, 108300 (3) | COL2A1 |
| Stickler syndrome, type II, 604841 (3) | COL11A1, STL2 |
| Stickler syndrome, type III, 184840 (3) | COL11A2, STL3, DFNA13 |
| Stomach cancer, 137215 (3) | KRAS2, RASK2 |
| Stroke, susceptibility to, 1, 606799 (3) | PDE4D, DPDE3, STRK1 |
| Stroke, susceptibility to, 601367 (3) | ALOX5AP, FLAP |
| Stuve-Wiedemann syndrome/Schwartz- | LIFR, STWS, SWS, SJS2 |
| Jampel type 2 syndrome, 601559 (3) | |
| Subcortical laminal heteropia, X-linked, | DCX, DBCN, LISX |
| 300067 (3) | |
| Subcortical laminar heterotopia (3) | PAFAH1B1, LIS1 |
| Succinic semialdehyde dehydrogenase | SSADH |
| deficiency (3) | |
| Sucrose intolerance (3) | SI |
| Sudden infant death with dysgenesis of the | TSPYL1, TSPYL, SIDDT |
| testes syndrome, 608800 (3) | |
| Sulfite oxidase deficiency, 272300 (3) | SUOX |
| Superoxide dismutase, elevated | SOD3 |
| extracellular (3) | |
| Supranuclear palsy, progressive, 601104 (3) | MAPT, MTBT1, DDPAC, MSTD |
| Supranuclear palsy, progressive atypical, | MAPT, MTBT1, DDPAC, MSTD |
| 260540 (3) | |
| Supravalvar aortic stenosis, 185500 (3) | ELN |
| Surfactant deficiency, neonatal, 267450 (3) | ABCA3, ABC3 |
| Surfactant protein C deficiency (3) | SFTPC, SFTP2 |
| Sutherland-Haan syndrome-like, 300465 (3) | ATRX, XH2, XNP, MRXS3, SHS |
| Sweat chloride elevation without CF (3) | CFTR, ABCC7, CF, MRP7 |
| Symphalangism, proximal, 185800 (3) | NOG, SYM1, SYNS1 |
| Syndactyly, type III, 186100 (3) | GJA1, CX43, ODDD, SDTY3, ODOD |
| Synostoses syndrome, multiple, 1, 186500 | NOG, SYM1, SYNS1 |
| (3) | |
| Synpolydactyly, 3/3′4, associated with | FBLN1 |
| metacarpal and metatarsal synostoses, | |
| 608180 (3) | |
| Synpolydactyly, type II, 186000 (3) | HOXD13, HOX4I, SPD |
| Synpolydactyly with foot anomalies, 186000 | HOXD13, HOX4I, SPD |
| (3) | |
| Systemic lupus erythematosus, | TNFSF6, APT1LG1, FASL |
| susceptibility, 152700 (3) | |
| Systemic lupus erythematosus, | DNASE1, DNL1 |
| susceptibility to, 152700 (3) | |
| Systemic lupus erythematosus, | PTPN8, PEP, PTPN22, LYP |
| susceptibility to, 152700 (3) | |
| Systemic lupus erythematosus, | PDCD1, SLEB2 |
| susceptibility to, 2, 605218, 152700 (3) | |
| Tall stature, susceptibility to (3) | MCM6 |
| Tangier disease, 205400 (3) | ABCA1, ABC1, HDLDT1, TGD |
| Tarsal-carpal coalition syndrome, 186570 | NOG, SYM1, SYNS1 |
| (3) | |
| Tauopathy and respiratory failure (3) | MAPT, MTBT1, DDPAC, MSTD |
| Tay-Sachs disease, 272800 (3) | HEXA, TSD |
| T-cell acute lymphoblastic leukemia (3) | BAX |
| T-cell immunodeficiency, congenital | WHN |
| alopecia, and nail dystrophy (3) | |
| T-cell prolymphocytic leukemia, sporadic (3) | ATM, ATA, AT1 |
| Temperature-sensitive apoptosis, cellular | DAD1 |
| (3) | |
| Tetra-amelia, autosomal recessive, 273395 | WNT3, INT4 |
| (3) | |
| Tetralogy of Fallot, 187500 (3) | JAG1, AGS, AHD |
| Tetralogy of Fallot, 187500 (3) | ZFPM2, FOG2 |
| Tetrology of Fallot, 187500 (3) | NKX2E, CSX |
| Thalassemia, alpha-(3) | HBA2 |
| Thalassemia-beta, dominant inclusion-body, | HBB |
| 603902 (3) | |
| Thalassemia, delta-(3) | HBD |
| Thalassemia due to Hb Lepore (3) | HBD |
| Thalassemia, Hispanic gamma-delta-beta | LCRB |
| (3) | |
| Thalassemias, alpha-(3) | HBA1 |
| Thalassemias, beta-(3) | HBB |
| Thanatophoric dysplasia, types I and II, | FGFR3, ACH |
| 187600 (3) | |
| Thiamine-responsive megaloblastic anemia | SLC19A2, THTR1 |
| syndrome, 249270 (3) | |
| Thrombocythemia, essential, 187950 (3) | JAK2 |
| Thrombocythemia, essential, 187950 (3) | THPO, MGDF, MPLLG, TPO |
| Thrombocytopenia-2, 188000 (3) | FLJ14813, THC2 |
| Thrombocytopenia, congenital | MPL, TPOR, MPLV |
| amegakaryocytic, 604498 (3) | |
| Thrombocytopenia, X-linked, 313900 (3) | WAS, IMD2, THC |
| Thrombocytopenia, X-linked, intermittent, | WAS, IMD2, THC |
| 313900 (3) | |
| Thromboembolism susceptibility due to | F5 |
| factor V Leiden (3) | |
| Thrombophilia due to factor V Liverpool (3) | F5 |
| Thrombophilia due to heparin cofactor II | HCF2, HC2, SERPIND1 |
| deficiency (3) | |
| Thrombophilia due to HRG deficiency (3) | HRG |
| Thrombophilia due to protein C deficiency | PROC |
| (3) | |
| Thrombophilia due to thrombomodulin | THBD, THRM |
| defect (3) | |
| Thrombophilia, dysfibrinogenemic (3) | FGB |
| Thrombophilia, dysfibrinogenemic (3) | FGG |
| Thrombosis, hyperhomocysteinemic (3) | CBS |
| Thrombotic thrombocytopenic purpura, | ADAMTS13, VWFCP, TTP |
| familial, 274150 (3) | |
| Thrombycytosis, susceptibility to, 187950 | MPL, TPOR, MPLV |
| (3) | |
| Thymine-uraciluria (3) | DPYD, DPD |
| Thyroid adenoma, hyperfunctioning (3) | TSHR |
| Thyroid carcinoma (3) | TP53, P53, LFS1 |
| Thyroid carcinoma, follicular, 188470 (3) | MINPP1, HIPER1 |
| Thyroid carcinoma, follicular, 188470 (3) | PTEN, MMAC1 |
| Thyroid carcinoma, follicular, somatic, | HRAS |
| 188470 (3) | |
| Thyroid carcinoma, papillary, 188550 (3) | GOLGA5, RFG5, PTC5 |
| Thyroid carcinoma, papillary, 188550 (3) | NCOA4, ELE1, PTC3 |
| Thyroid carcinoma, papillary, 188550 (3) | PCM1, PTC4 |
| Thyroid carcinoma, papillary, 188550 (3) | PRKAR1A, TSE1, CNC1, CAR |
| Thyroid carcinoma, papillary, 188550 (3) | TIF1G, RFG7, PTC7 |
| Thyroid carcinoma, papillary, 188550 (3) | TRIM24, TIF1, TIF1A, PTC6 |
| Thyroid hormone organification defect IIA, | TPO, TPX |
| 274500 (3) | |
| Thyroid hormone resistance, 188570 (3) | THRB, ERBA2, THR1 |
| Thyroid hormone resistance, autosomal | THRB, ERBA2, THR1 |
| recessive, 274300 (3) | |
| Thyrotoxic periodic paralysis, susceptibility | CACNA1S, CACNL1A3, CCHL1A3 |
| to, 188580 (3) | |
| Thyrotropin-releasing hormone resistance, | TRHR |
| generalized (3) | |
| Thyroxine-binding globulin deficiency (3) | TBG |
| Tietz syndrome, 103500 (3) | MITF, WS2A |
| Timothy syndrome, 601005 (3) | CACNA1C, CACNL1A1, CCHL1A1, TS |
| Toenail dystrophy, isolated, 607523 (3) | COL7A1 |
| Tolbutamide poor metabolizer (3) | CYP2C9 |
| Total iodide organification defect, 274500 | TPO, TPX |
| (3) | |
| Townes-Brocks branchiootorenal-like | SALL1, HSAL1, TBS |
| syndrome, 107480 (3) | |
| Townes-Brocks syndrome, 107480 (3) | SALL1, HSAL1, TBS |
| Transaldolase deficiency, 606003 (3) | TALDO1 |
| Transcobalamin II deficiency (3) | TCN2, TC2 |
| Transient bullous of the newborn, 131705 | COL7A1 |
| (3) | |
| Transposition of great arteries, dextro- | CFC1, CRYPTIC, HTX2 |
| looped, 217095 (3) | |
| Transposition of the great arteries, dextro- | THRAP2, PROSIT240, TRAP240L, |
| looped, 608808 (3) | KIAA1025 |
| Treacher Collins mandibulofacial | TCOF1, MFD1 |
| dysostosis, 154500 (3) | |
| Tremor, familial essential, 2, 602134 (3) | HS1BP3, FLJ14249, ETM2 |
| Trichodontoosseous syndrome, 190320 (3) | DLX3, TDO |
| Trichorhinophalangeal syndrome, type I, | TRPS1 |
| 190350 (3) | |
| Trichorhinophalangeal syndrome, type III, | TRPS1 |
| 190351 (3) | |
| Trichothiodystrophy (3) | ERCC3, XPB |
| Trichothiodystrophy, 601675 (3) | ERCC2, EM9 |
| Trichothiodystrophy, complementation | TGF2H5, TTDA, TFB5, C6orf175 |
| group A, 601675 (3) | |
| Trichothiodystrophy, nonphotosensitive 1, | TTDN1, C7orf11, ABHS |
| 234050 (3) | |
| Trifunctional protein deficiency, type 1 (3) | HADHA, MTPA |
| Trifunctional protein deficiency, type II (3) | HADHB |
| Trismus-pseudocomptodactyly syndrome, | MYH8 |
| 158300 (3) | |
| Tropical calcific pancreatitis, 608189 (3) | SPINK1, PSTI, PCTT, TATI |
| Troyer syndrome, 275900 (3) | SPG20 |
| TSC2 angiomyolipomas, renal, modifier of, | IFNG |
| 191100 (3) | |
| Tuberculosis, susceptibility to (3) | IFNGR1 |
| Tuberculosis, susceptibility to, 607948 (3) | IFNG |
| Tuberous sclerosis-1, 191100 (3) | TSC1, LAM |
| Tuberous sclerosis-2, 191100 (3) | TSC2, LAM |
| Turcot syndrome, 276300 (3) | APC, GS, FPC |
| Turcot syndrome with glioblastoma, 276300 | MLH1, COCA2, HNPCC2 |
| (3) | |
| Turcot syndrome with glioblastoma, 276300 | PMS2, PMSL2, HNPCC4 |
| (3) | |
| Twinning, dizygotic, 276400 (3) | FSHR, ODG1 |
| Tyrosinemia, type I (3) | FAH |
| Tyrosinemia, type II (3) | TAT |
| Tyrosinemia, type III (3) | HPD |
| Ullrich congenital muscular dystrophy, | COL6A1, OPLL |
| 254090 (3) | |
| Ullrich congenital muscular dystrophy, | COL6A3 |
| 254090 (3) | |
| Ullrich scleroatonic muscular dystrophy, | COL6A2 |
| 254090 (3) | |
| Ulnar-mammary syndrome, 181450 (3) | TBX3 |
| Unipolar depression, susceptibility to, | TPH2, NTPH |
| 608516 (3) | |
| Unna-Thost disease, nonepidermolytic, | KRT1 |
| 600962 (3) | |
| Urolithiasis, 2,8-dihydroxyadenine (3) | APRT |
| Urolithiasis, hypophosphatemic (3) | SLC17A2, NPT2 |
| Usher syndrome, type 1B (3) | MYO7A, USH1B, DFNB2, DFNA11 |
| Usher syndrome, type 1C, 276904 (3) | USH1C, DFNB18 |
| Usher syndrome, type 1D, 601067 (3) | CDH23, USH1D |
| Usher syndrome, type 1F, 602083 (3) | PCDH15, DFNB23 |
| Usher syndrome, type 1G, 606943 (3) | SANS, USH1G |
| Usher syndrome, type 2A, 276901 (3) | USH2A |
| Usher syndrome, type 3, 276902 (3) | USH3A, USH3 |
| Usher syndrome, type IIC, 605472 (3) | MASS1, VLGR1, KIAA0686, FEB4, |
| | USH2C |
| Uterine leiomyoma (3) | HMGA2, HMGIC, BABL, LIPO |
| UV-induced skin damage, vulnerability to (3) | MC1R |
| van Buchem disease, type 2, 607636 (3) | LRP5, BMND1, LRP7, LR3, OPPG, |
| | VBCH2 |
| van der Woude syndrome, 119300 (3) | IRF6, VWS, LPS, PIT, PPS, OFC6 |
| VATER association with hydrocephalus, | PTEN, MMAC1 |
| 276950 (3) | |
| Velocardiofacial syndrome, 192430 (3) | TBX1, DGS, CTHM, CAFS, TGA, |
| | DORV, VCFS, DGCR |
| Venous malformations, multiple cutaneous | TEK, TIE2, VMCM |
| and mucosal, 600195 (3) | |
| Venous thrombosis, susceptibility to (3) | SERPINA10, ZPI |
| Ventricular fibrillation, idiopathic, 603829 (3) | SCN5A, LQT3, IVF, HB1, SSS1 |
| Ventricular tachycardia, idiopathic, 192605 | GNAI2, GNAI2B, GIP |
| (3) | |
| Ventricular tachycardia, stress-induced | CASQ2 |
| polymorphic, 604772 (3) | |
| Ventricular tachycardia, stress-induced | RYR2, VTSIP |
| polymorphic, 604772 (3) | |
| Vertical talus, congenital, 192950 (3) | HOXD10, HOX4D |
| Viral infections, recurrent (3) | FCGR3A, CD16, IGFR3 |
| Viral infection, susceptibility to (3) | OAS1, OIAS |
| Virilization, maternal and fetal, from | CYP19A1, CYP19, ARO |
| placental aromatase deficiency (3) | |
| Vitamin K-dependent clotting factors, | VKORC1, VKOR, VKCFD2, FLJ00289 |
| combined deficiency of, 2, 607473 (3) | |
| Vitamin K-dependent coagulation defect, | GGCX |
| 277450 (3) | |
| Vitelliform macular dystrophy, adult-onset, | VMD2 |
| 608161 (3) | |
| VLCAD deficiency, 201475 (3) | ACADVL, VLCAD |
| Vohwinkel syndrome, 124500 (3) | GJB2, CX26, DFNB1, PPK, DFNA3, |
| | KID, HID |
| Vohwinkel syndrome with ichthyosis, | LOR |
| 604117 (3) | |
| von Hippel-Lindau disease, modification of, | CCND1, PRAD1, BCL1 |
| 193300 (3) | |
| von Hippel-Lindau syndrome, 193300 (3) | VHL |
| von Willebrand disease (3) | VWF, F8VWF |
| Waardenburg-Shah syndrome, 277580 (3) | EDNRB, HSCR2, ABCDS |
| Waardenburg-Shah syndrome, 277580 (3) | SOX10, WS4 |
| Waardenburg syndrome/albinism, digenic, | TYR |
| 103470 (3) | |
| Waardenburg syndrome/ocular albinism, | MITF, WS2A |
| digenic, 103470 (3) | |
| Waardenburg syndrome, type I, 193500 (3) | PAX3, WS1, HUP2, CDHS |
| Waardenburg syndrome, type IIA, 193510 | MITF, WS2A |
| (3) | |
| Waardenburg syndrome, type III, 148820 (3) | PAX3, WS1, HUP2, CDHS |
| Waardenburg syndrome, typ IID, 608890 (3) | SNAI2, SLUG, WS2D |
| Wagner syndrome, 143200 (3) | COL2A1 |
| WAGR syndrome, 194072 (3) | WT1 |
| Walker-Warburg syndrome, 236670 (3) | FCMD |
| Walker-Warburg syndrome, 236670 (3) | POMT1 |
| Warburg micro syndrome 1, 600118 (3) | RAB3GAP, WARBM1, P130 |
| Warfarin resistance, 122700 (3) | VKORC1, VKOR, VKCFD2, FLJ00289 |
| Warfarin sensitivity, 122700 (3) | CYP2C9 |
| Warfarin sensitivity (3) | F9, HEMB |
| Watson syndrome, 193520 (3) | NF1, VRNF, WSS, NFNS |
| Weaver syndrome, 277590 (3) | NSD1, ARA267, STO |
| Wegener-like granulomatosis (3) | TAP2, ABCB3, PSF2, RING11 |
| Weill-Marchesani syndrome, dominant, | FBN1, MFS1, WMS |
| 608328 (3) | |
| Weill-Marchesani syndrome, recessive, | ADAMTS10, WMS |
| 277600 (3) | |
| Weissenbacher-Zweymuller syndrome, | COL11A2, STL3, DFNA13 |
| 277610 (3) | |
| Werner syndrome, 277700 (3) | RECQL2, RECQ3, WRN |
| Wernicke-Korsakoff syndrome, susceptibility | TKT |
| to, 277730 (3) | |
| Weyers acrodental dysostosis, 193530 (3) | EVC |
| WHIM syndrome, 193670 (3) | CXCR4, D2S201E, NPY3R, WHIM |
| White sponge nevus, 193900 (3) | KRT13 |
| White sponge nevus, 193900 (3) | KRT4, CYK4 |
| Williams-Beuren syndrome, 194050 (3) | ELN |
| Wilms tumor, 194070 (3) | BRCA2, FANCD1 |
| Wilms tumor, somatic, 194070 (3) | GPC3, SDYS, SGBS1 |
| Wilms tumor susceptibility-5, 601583 (3) | POU6F2, WTSL, WT5 |
| Wilms tumor, type 1, 194070 (3) | WT1 |
| Wilson disease, 277900 (3) | ATP7B, WND |
| Wiskott-Aldrich syndrome, 301000 (3) | WAS, IMD2, THC |
| Witkop syndrome, 189500 (3) | MSX1, HOX7, HYD1, OFC5 |
| Wolcott-Rallison syndrome, 226980 (3) | EIF2AK3, PEK, PERK, WRS |
| Wolff-Parkinson-White syndrome, 194200 | PRKAG2, WPWS |
| (3) | |
| Wolfram syndrome, 222300 (3) | WFS1, WFRS, WFS, DFNA6 |
| Wolman disease (3) | LIPA |
| Xanthinuria, type I, 278300 (3) | XDH |
| Xeroderma pigmentosum, group A (3) | XPA |
| Xeroderma pigmentosum, group B (3) | ERCC3, XPB |
| Xeroderma pigmentosum, group C (3) | XPC, XPCC |
| Xeroderma pigmentosum, group D, 278730 | ERCC2, EM9 |
| (3) | |
| Xeroderma pigmentosum, group E, DDB- | DDB2 |
| negative subtype, 278740 (3) | |
| Xeroderma pigmentosum, group F, 278760 | ERCC4, XPF |
| (3) | |
| Xeroderma pigmentosum, group G, 278780 | ERCC5, XPG |
| (3) | |
| Xeroderma pigmentosum, variant type, | POLH, XPV |
| 278750 (3) | |
| X-inactivation, familial skewed, 300087 (3) | XIC, XCE, XIST, SXI1 |
| XLA and isolated growth hormone | BTK, AGMX1, IMD1, XLA, AT |
| deficiency, 307200 (3) | |
| Yellow nail syndrome, 153300 (3) | FOXC2, FKHL14, MFH1 |
| Yemenite deaf-blind hypopigmentation | SOX10, WS4 |
| syndrome, 601706 (3) | |
| Zellweger syndrome-1, 214100 (3) | PEX1, ZWS1 |
| Zellweger syndrome, 214100 (3) | PEX10, NALD |
| Zellweger syndrome, 214100 (3) | PEX13, ZWS, NALD |
| Zellweger syndrome, 214100 (3) | PEX14 |
| Zellweger syndrome, 214100 (3) | PEX26 |
| Zellweger syndrome, 214100 (3) | PXF, HK33, D1S2223E, PEX19 |
| Zellweger syndrome, 214100 (3) | PXR1, PEX5, PTS1R |
| Zellweger syndrome-2 (3) | ABCD3, PXMP1, PMP70 |
| Zellweger syndrome-3 (3) | PXMP3, PAF1, PMP35, PEX2 |
| Zellweger syndrome, complementation | PEX16 |
| group 9 (3) | |
| Zellweger syndrome, complementation | PEX3 |
| group G, 214100 (3) | |
| Zlotogora-Ogur syndrome, 225000 (3) | HVEC, PVRL1, PVRR1, PRR1 |

Further non-limiting examples of disease models created by a method of the invention include a Parkinson's disease model, an addiction model, an inflammation model, a cardiovascular disease model, an Alzheimer's disease model, an autism spectrum disorder model, a macular degeneration model, a schizophrenia model, a tumor suppression model, a trinucleotide repeat disorder model, a neurotransmission disorder model, a secretase-associated disorder model, an ALS model, a prion disease model, on ABC transporter protein—associated disorder model, and an immunodeficiency model. Each is discussed in more detail below.

### A. Parkinsons Disease

In one embodiment, a method of the invention may be used to create an animal or cell in which at least one chromosomal sequence associated with Parkinsons disease (PD) has been edited. Suitable chromosomal edits may include, but are not limited to, the type of edits detailed in section I(f) above.

In some embodiments, one or more chromosomal sequences encoding a protein or control sequence associated with PD may be edited. A PD-associated protein or control sequence may typically be selected based on an experimental association of the PD-associated protein or control sequence to PD. By way of non-limiting example, the production rate or circulating concentration of a PD-related protein may be elevated or depressed in a population having PD relative to a population not having PD. Differences in protein levels may be assessed using proteomic or genomic analysis techniques known in the art. By way of non-limiting example, proteins associated with Parkinson's disease include but are not limited to α-synuclein, DJ-1, LRRK2, PINK1, Parkin, UCHL1, Synphilin-1, and NURR1.

In certain embodiments, an animal created by a method of the invention may be used to study the effects of mutations on the animal and development and/or progression of PD using measures commonly used in the study of PD. Methods for measuring and studying progression of PD in animals are known in the art. Commonly used measures in the study of PD may include without limit, amyloidogenesis or protein aggregation, dopamine response, neurodegeneration, development of mitochondrial related dysfunction phenotypes, as well as functional, pathological or biochemical assays. Other relevant indicators regarding development or progression of PD include coordination, balance, gait, motor impairment, tremors and twitches, rigidity, hypokinesia, and cognitive impairments. Such assays may be made in comparison to wild type littermates.

### B. Addiction

Addiction, as used herein, is defined as a chronic disease of brain reward, motivation, memory, and related neuronal circuitry contained within various brain structures. Specific examples of brain structures that may experience dysfunction associated with an addiction disorder include nucleus accumbens, ventral pallidum, dorsal thalamus, prefrontal cortex, striatum, substantia nigra, pontine reticular formation, amygdala, and ventral tegmental area. Dysfunction in these neural circuits may lead to various biological, psychological, social and behavioral symptoms of addiction.

Biological symptoms of addiction may include overproduction or underproduction of one or more addiction-related proteins; redistribution of one or more addiction-related proteins within the brain; the development of tolerance, reverse tolerance, or other changes in sensitivity to the effects of an addictive substance or a neurotransmitter within the brain; high blood pressure; and withdrawal symptoms such as insomnia, restlessness, loss of appetite, depression, weakness, irritability, anger, pain, and craving.

Psychological symptoms of addiction may vary depending on the particular addictive substance and the duration of the addiction. Non-limiting examples of psychological symptoms of addiction include mood swings, paranoia, insomnia, psychosis, schizophrenia, tachycardia panic attacks, cognitive impairments, and drastic changes in the personality that can lead to aggressive, compulsive, criminal and/or erratic behaviors.

Social symptoms of addiction may include low self-esteem, verbal hostility, ignorance of interpersonal means, focal anxiety such as fear of crowds, rigid interpersonal behavior, grossly bizarre behavior, rebelliousness, and diminished recognition of significant problems with an individual's behaviors and interpersonal relationships.

Non-limiting examples of behavioral symptoms of addiction include impairment in behavioral control, inability to consistently abstain from the use of addictive substances, cycles of relapse and remission, risk-taking behavior, pleasure-seeking behavior, novelty-seeking behavior, relief-seeking behavior, and reward-seeking behavior.

Addictions may be substance addictions typically associated with the ingestion of addictive substances. Addictive substances may include psychoactive substances capable of crossing the blood-brain barrier and temporarily altering the chemical milieu of the brain. Non-limiting examples of addictive substances include alcohol; opioid compounds such as opium and heroin; sedative, hypnotic, or anxiolytic compounds such as benzodiazepine and barbiturate compounds; cocaine and related compounds; cannabis and related compounds; amphetamine and amphetamine-like compounds; hallucinogen compounds; inhalants such as glue or aerosol propellants; phencyclidine or phencyclidine-like compounds; and nicotine. In addition, addictions may be behavioral addictions associated with compulsions that are not substance-related, such as problem gambling and computer addiction.

In one embodiment, a method of the invention may be used to create an animal or cell in which at least one addiction-related chromosomal sequence has been edited. Suitable edits may include, but are not limited to, the type of edits detailed in section I(f) above.

Addiction-related nucleic acid sequences are a diverse set of sequences associated with susceptibility for developing an addiction, the presence of an addiction, the severity of an addiction or any combination thereof. An addiction-related nucleic acid sequence may typically be selected based on an experimental association of the addiction-related nucleic acid sequence to an addiction disorder. An addiction-related nucleic acid sequence may encode an addiction-related protein or may be an addiction-related control sequence. By way of non-limiting example, the production rate or circulating concentration of an addiction-related protein may be elevated or depressed in a population having an addiction disorder relative to a population lacking the addiction disorder. Differences in protein levels may be assessed using proteomic or genomic analysis techniques known in the art.

Non-limiting examples of addiction-related proteins include ABAT (4-aminobutyrate aminotransferase); ACN9 (ACN9 homolog (S. cerevisae)); ADCYAP1 (Adenylate cyclase activating polypeptide 1); ADH1B (Alcohol dehydrogenase IB (class I), beta polypeptide); ADH1C (Alcohol dehydrogenase 1C (class I), gamma polypeptide); ADH4 (Alcohol dehydrogenase 4); ADH7 (Alcohol dehydrogenase 7 (class IV), mu or sigma polypeptide); ADORA1 (Adenosine A1 receptor); ADRA1A (Adrenergic, alpha-1A-, receptor); ALDH2 (Aldehyde dehydrogenase 2 family); ANKK1 (Ankyrin repeat, TaqI A1 allele); ARC (Activity-regulated cytoskeleton-associated protein); ATF2 (Corticotrophin-releasing factor); AVPR1A (Arginine vasopressin receptor 1A); BDNF (Brain-derived neurotrophic factor); BMAL1 (Aryl hydrocarbon receptor nuclear translocator-like); CDK5 (Cyclin-dependent kinase 5); CHRM2 (Cholinergic receptor, muscarinic 2); CHRNA3 (Cholinergic receptor, nicotinic, alpha 3); CHRNA4 (Cholinergic receptor, nicotinic, alpha 4); CHRNA5 (Cholinergic receptor, nicotinic, alpha 5); CHRNA7 (Cholinergic receptor, nicotinic, alpha 7); CHRNB2 (Cholinergic receptor, nicotinic, beta 2); CLOCK (Clock homolog (mouse)); CNR1 (Cannabinoid receptor 1); CNR2 (Cannabinoid receptor type 2); COMT (Catechol-O-methyltransferase); CREB1 (cAMP Responsive element binding protein 1); CREB2 (Activating transcription factor 2); CRHR1 (Corticotropin releasing hormone receptor 1); CRY1 (Cryptochrome 1); CSNK1E (Casein kinase 1, epsilon); CSPG5 (Chondroitin sulfate proteoglycan 5); CTNNB1 (Catenin (cadherin-associated protein), beta 1, 88 kDa); DBI (Diazepam binding inhibitor); DDN (Dendrin); DRD1 (Dopamine receptor D1); DRD2 (Dopamine receptor D2); DRD3 (Dopamine receptor D3); DRD4 (Dopamine receptor D4); EGR1 (Early growth response 1); ELTD1 (EGF, latrophilin and seven transmembrane domain containing 1); FAAH (Fatty acid amide hydrolase); FOSB (FBJ murine osteosarcoma viral oncogene homolog); FOSB (FBJ murine osteosarcoma viral oncogene homolog B); GABBR2 (Gamma-aminobutyric acid (GABA) B receptor, 2); GABRA2 (Gamma-aminobutyric acid (GABA) A receptor, alpha 2); GABRA4 (Gamma-aminobutyric acid (GABA) A receptor, alpha 4); GABRA6 (Gamma-aminobutyric acid (GABA) A receptor, alpha 6); GABRB3 (Gamma-aminobutyric acid (GABA) A receptor, alpha 3); GABRE (Gamma-aminobutyric acid (GABA) A receptor, epsilon); GABRG1 (Gamma-aminobutyric acid (GABA) A receptor, gamma 1); GAD1 (Glutamate decarboxylase 1); GAD2 (Glutamate decarboxylase 2); GAL (Galanin prepropeptide); GDNF (Glial cell derived neurotrophic factor); GRIA1 (Glutamate receptor, ionotropic, AMPA 1); GRIA2 (Glutamate receptor, ionotropic, AMPA 2); GRIN1 (Glutamate receptor, ionotropic, N-methyl D-aspartate 1); GRIN2A (Glutamate receptor, ionotropic, N-methyl D-aspartate 2A); GRM2 (Glutamate receptor, metabotropic 2, mGluR2); GRM5 (Metabotropic glutamate receptor 5); GRM6 (Glutamate receptor, metabotropic 6); GRM8 (Glutamate receptor, metabotropic 8); HTR1B (5-Hydroxytryptamine (serotonin) receptor 1B); HTR3A (5-Hydroxytryptamine (serotonin) receptor 3A); IL1 (Interleukin 1); IL15 (Interleukin 15); ILIA (Interleukin 1 alpha); IL1B (Interleukin 1 beta); KCNMA1 (Potassium large conductance calcium-activated channel, subfamily M, alpha member 1); LGALS1 (lectin galactoside-binding soluble 1); MAOA (Monoamine oxidase A); MAOB (Monoamine oxidase B); MAPK1 (Mitogen-activated protein kinase 1); MAPK3 (Mitogen-activated protein kinase 3); MBP (Myelin basic protein); MC2R (Melanocortin receptor type 2); MGLL (Monoglyceride lipase); MOBP (Myelin-associated oligodendrocyte basic protein); NPY (Neuropeptide Y); NR4A1 (Nuclear receptor subfamily 4, group A, member 1); NR4A2 (Nuclear receptor subfamily 4, group A, member 2); NRXN1 (Neurexin 1); NRXN3 (Neurexin 3); NTRK2 (Neurotrophic tyrosine kinase, receptor, type 2); NTRK2 (Tyrosine kinase B neurotrophin receptor); OPRD1 (delta-Opioid receptor); OPRK1 (kappa-Opioid receptor); OPRM1 (mu-Opioid receptor); PDYN (Dynorphin); PENK (Enkephalin); PER2 (Period homolog 2 (Drosophila)); PKNOX2 (PBX/knotted 1 homeobox 2); PLP1 (Proteolipid protein 1); POMC (Proopiomelanocortin); PRKCE (Protein kinase C, epsilon); PROKR2 (Prokineticin receptor 2); RGS9 (Regulator of G-protein signaling 9); RIMS2 (Regulating synaptic membrane exocytosis 2); SCN9A (sodium channel voltage-gated type IX alpha subunit); SLC17A6 (Solute carrier family 17 (sodium-dependent inorganic phosphate cotransporter), member 6); SLC17A7 (Solute carrier family 17 (sodium-dependent inorganic phosphate cotransporter), member 7); SLC1A2 (Solute carrier family 1 (glial high affinity glutamate transporter), member 2); SLC1A3 (Solute carrier family 1 (glial high affinity glutamate transporter), member 3); SLC29A1 (solute carrier family 29 (nucleoside transporters), member 1); SLC4A7 (Solute carrier family 4, sodium bicarbonate cotransporter, member 7); SLC6A3 (Solute carrier family 6 (neurotransmitter transporter, dopamine), member 3); SLC6A4 (Solute carrier family 6 (neurotransmitter transporter, serotonin), member 4); SNCA (Synuclein, alpha (non A4 component of amyloid precursor)); TFAP2B (Transcription factor AP-2 beta); and TRPV1 (Transient receptor potential cation channel, subfamily V, member 1).

Preferred addiction-related proteins may include ABAT (4-aminobutyrate aminotransferase), DRD2 (Dopamine receptor D2), DRD3 (Dopamine receptor D3), DRD4 (Dopamine receptor D4), GRIA1 (Glutamate receptor, ionotropic, AMPA 1), GRIA2 (Glutamate receptor, ionotropic, AMPA 2), GRIN1 (Glutamate receptor, ionotropic, N-methyl D-aspartate 1), GRIN2A (Glutamate receptor, ionotropic, N-methyl D-aspartate 2A), GRM5 (Metabotropic glutamate receptor 5), HTR1B (5-Hydroxytryptamine (serotonin) receptor 1B), PDYN (Dynorphin), PRKCE (Protein kinase C, epsilon), LGALS1 (lectin galactoside-binding soluble 1), TRPV1 (transient receptor potential cation channel subfamily V member 1), SCN9A (sodium channel voltage-gated type IX alpha subunit), OPRD1 (opioid receptor delta 1), OPRK1 (opioid receptor kappa 1), OPRM1 (opioid receptor mu 1), and any combination thereof.

In certain embodiments, an animal created by a method of the invention may be used as a model for indications of addiction disorders by comparing the measurements of an assay obtained from a genetically modified animal comprising at least one edited chromosomal sequence encoding an addiction-related protein to the measurements of the assay using a wild-type animal. Non-limiting examples of assays used to assess for indications of an addictive disorder include behavioral assays, physiological assays, whole animal assays, tissue assays, cell assays, biomarker assays, and combinations thereof. The indications of addiction disorders may occur spontaneously, or may be promoted by exposure to exogenous agents such as addictive substances or addiction-related proteins. Alternatively, the indications of addiction disorders may be induced by withdrawal of an addictive substance or other compound such as an exogenously administered addiction-related protein.

An additional aspect of the present disclosure encompasses a method of assessing the efficacy of a treatment for inhibiting addictive behaviors and/or reducing withdrawal symptoms of a genetically modified animal comprising at least one edited chromosomal sequence associated with addiction. Treatments for addiction that may be assessed include the administering of one or more novel candidate therapeutic compounds, a novel combination of established therapeutic compounds, a novel therapeutic method, and any combination thereof. Novel therapeutic methods may include various drug delivery mechanisms, nanotechnology applications in drug therapy, surgery, and combinations thereof.

Behavioral testing of a genetically modified animal comprising at least one edited addiction-related protein and/or a wild-type animal may be used to assess the side effects of a therapeutic compound or combination of therapeutic agents. The genetically modified animal and optionally a wild-type animal may be treated with the therapeutic compound or combination of therapeutic agents and subjected to behavioral testing. The behavioral testing may assess behaviors including but not limited to learning, memory, anxiety, depression, addiction, and sensory-motor functions.

An additional aspect provides a method for assessing the therapeutic potential of an agent in an animal that may include contacting a genetically modified animal comprising at least one edited chromosomal sequence encoding an addiction-related protein, and comparing results of a selected parameter to results obtained from a wild-type animal with no contact with the same agent. Selected parameters include but are not limited to a) spontaneous behaviors; b) performance during behavioral testing; c) physiological anomalies; d) abnormalities in tissues or cells; e) biochemical function; and f) molecular structures.

### C. Inflammation

In one embodiment, a method of the invention may be used to create an animal or cell in which at least one chromosomal sequence associated with inflammation has been edited. Suitable chromosomal edits may include, but are not limited to, the type of edits detailed in section I(f) above.

In each of the above embodiments, one or more chromosomal sequences associated with inflammation may be edited. An inflammation-related chromosomal sequence may typically be selected based on an experimental association of the inflammation-related sequence to an inflammation disorder. An inflammation-related sequence may encode an inflammation-related protein or may be an inflammation-related control sequence. For example, the production rate or circulating concentration of an inflammation-related protein may be elevated or depressed in a population having an inflammation disorder relative to a population lacking the inflammation disorder. Differences in protein levels may be assessed using proteomic or genomic analysis techniques known in the art.

Non-limiting examples of inflammation-related proteins whose chromosomal sequence may be edited include the monocyte chemoattractant protein-1 (MCP1) encoded by the Ccr2 gene, the C-C chemokine receptor type 5 (CCR5) encoded by the Ccr5 gene, the IgG receptor IIB (FCGR2b, also termed CD32) encoded by the Fcgr2b gene, the Fc epsilon R1g (FCER1g) protein encoded by the Fcer1g gene, the forkhead box N1 transcription factor (FOXN1) encoded by the FOXN1 gene, Interferon-gamma (IFN-γ) encoded by the IFNg gene, interleukin 4 (IL-4) encoded by the IL-4 gene, perforin-1 encoded by the PRF-1 gene, the cyclooxygenase 1 protein (COX1) encoded by the COX1 gene, the cyclooxygenase 2 protein (COX2) encoded by the COX2 gene, the T-box transcription factor (TBX21) protein encoded by the TBX21 gene, the SH2-B PH domain containing signaling mediator 1 protein (SH2BPSM1) encoded by the SH2B1 gene (also termed SH2BPSM1), the fibroblast growth factor receptor 2 (FGFR2) protein encoded by the FGFR2 gene, the solute carrier family 22 member 1 (SLC22A1) protein encoded by the OCT1 gene (also termed SLC22A1), the peroxisome proliferator-activated receptor alpha protein (PPAR-alpha, also termed the nuclear receptor subfamily 1, group C, member 1; NR1C1) encoded by the PPARA gene, the phosphatase and tensin homolog protein (PTEN) encoded by the PTEN gene, interleukin 1 alpha (IL-1α) encoded by the IL-1A gene, interleukin 1 beta (IL-1β) encoded by the IL-1B gene, interleukin 6 (IL-6) encoded by the IL-6 gene, interleukin 10 (IL-10) encoded by the IL-10 gene, interleukin 12 alpha (IL-12a) encoded by the IL-12A gene, interleukin 12 beta (IL-12β) encoded by the IL-12B gene, interleukin 13 (IL-13) encoded by the IL-13 gene, interleukin 17A(IL-17A, also termed CTLA8) encoded by the IL-17A gene, interleukin 17B (IL-17B) encoded by the IL-17B gene, interleukin 17C (IL-170) encoded by the IL-17C gene interleukin 17D (IL-17D) encoded by the IL-17D gene interleukin 17F (IL-17F) encoded by the IL-17F gene, interleukin 23 (IL-23) encoded by the IL-23 gene, the chemokine (C-X3-C motif) receptor 1 protein (CX3CR1) encoded by the CX3CR1 gene, the chemokine (C-X3-C motif) ligand 1 protein (CX3CL1) encoded by the CX3CL1 gene, the recombination activating gene 1 protein (RAG1) encoded by the RAG1 gene, the recombination activating gene 2 protein (RAG2) encoded by the RAG2 gene, the protein kinase, DNA-activated, catalytic polypeptide1 (PRKDC) encoded by the PRKDC (DNAPK) gene, the protein tyrosine phosphatase non-receptor type 22 protein (PTPN22) encoded by the PTPN22 gene, tumor necrosis factor alpha (TNFα) encoded by the TNFA gene, the nucleotide-binding oligomerization domain containing 2 protein (NOD2) encoded by the NOD2 gene (also termed CARD15), or the cytotoxic T-lymphocyte antigen 4 protein (CTLA4, also termed CD152) encoded by the CTLA4 gene.

In certain embodiments, an animal created by a method of the invention may be used to study the effects of mutations on the animal and development and/or progression of inflammation using measures commonly used in the study of inflammation. Alternatively, an animal created by a method of the invention may be used to study the effects of the mutations on the progression of a disease state or disorder associated with inflammation-related proteins using measures commonly used in the study of said disease state or disorder. Non-limiting examples of measures that may be used include spontaneous behaviors of the genetically modified animal, performance during behavioral testing, physiological anomalies, differential responses to a compound, abnormalities in tissues or cells, and biochemical or molecular differences between genetically modified animals and wild type animals.

### D. Cardiovascular Disease

Cardiovascular diseases generally include high blood pressure, heart attacks, heart failure, and stroke and TIA. In one embodiment, a method of the invention may be used to create an animal or cell in which at least one chromosomal sequence associated with cardiovascular disease has been edited. Suitable chromosomal edits may include, but are not limited to, the type of edits detailed in section I(f) above.

Any chromosomal sequence involved in cardiovascular disease or the protein encoded by any chromosomal sequence involved in cardiovascular disease may be utilized in a method of the invention. A cardiovascular-related sequence may typically be selected based on an experimental association of the cardiovascular-related sequence to the development of cardiovascular disease. A cardiovascular-related nucleic acid sequence may encode a cardiovascular-related protein or may be a cardiovascular-related control sequence. For example, the production rate or circulating concentration of a cardiovascular-related protein may be elevated or depressed in a population having a cardiovascular disorder relative to a population lacking the cardiovascular disorder. Differences in protein levels may be assessed using proteomic or genomic analysis techniques known in the art.

By way of example, the chromosomal sequence may comprise, but is not limited to, IL1B (interleukin 1, beta), XDH (xanthine dehydrogenase), TP53 (tumor protein p53), PTGIS (prostaglandin I2 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP(C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), IL1A (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein IIIa, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C-X-C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), URI (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABIN1 (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member 1B), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PECAM1 (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PRO51 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-O-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (8321 placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHCl (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1B (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity IIIa, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor H1), NR1I2 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDX5 (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine:polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adenovirus E1B 19 kDa interacting protein 3), GCKR (glucokinase (hexokinase 4) regulator), S100A12 (S100 calcium binding protein A12), PADI4 (peptidyl arginine deiminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine N1-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RHOB (ras homolog gene family, member B), LY96 (lymphocyte antigen 96), FOXO1 (forkhead box O1), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15-Sep (15 kDa selenoprotein), CILP2 (cartilage intermediate layer protein 2), TERC (telomerase RNA component), GGT2 (gamma-glutamyltransferase 2), MT-001 (mitochondrially encoded cytochrome c oxidase I), and UOX (urate oxidase, pseudogene).

In an additional embodiment, the chromosomal sequence may further be selected from Pon1 (paraoxonase 1), LDLR (LDL receptor), ApoE (Apolipoprotein E), Apo B-100 (Apolipoprotein B-100), ApoA (Apolipoprotein(a)), ApoA1 (Apolipoprotein A1), CBS (Cystathione B-synthase), Glycoprotein IIb/IIb, MTHRF (5,10-methylenetetrahydrofolate reductase (NADPH), and combinations thereof. In one iteration, the chromosomal sequences and proteins encoded by chromosomal sequences involved in cardiovascular disease may be chosen from Cacna1C, Sod1, Pten, Ppar(alpha), Apo E, Leptin, and combinations thereof.

In certain embodiments, an animal created by a method of the invention may be used to study the effects of mutations on the animal and development and/or progression of cardiovascular disease using measures commonly used in the study of cardiovascular disease. For instance, suitable disease measures may include behavioral, electrophysiological, neurochemical, biochemical, or cellular dysfunctions which can be evaluated using any of a number of well-known diagnostic tests or assays.

### E. Alzheimer's Disease

In one embodiment, a method of the invention may be used to create an animal or cell in which at least one chromosomal sequence associated with Alheimer's disease (AD) has been edited. Suitable chromosomal edits may include, but are not limited to, the type of edits detailed in section I(f) above.

In some embodiments, one or more chromosomal sequences associated with AD may be edited. The AD-related nucleic acid sequence may typically be selected based on an experimental association of the AD-related nucleic acid sequence to an AD disorder. An AD-related nucleic acid sequence may encode an AD-related protein or may be an AD-related control sequence. For example, the production rate or circulating concentration of an AD-related protein may be elevated or depressed in a population having an AD disorder relative to a population lacking the AD disorder. Differences in protein levels may be assessed using proteomic or genomic analysis techniques known in the art.

By way of non-limiting example, proteins associated with AD include but are not limited to the very low density lipoprotein receptor protein (VLDLR) encoded by the VLDLR gene, the ubiquitin-like modifier activating enzyme 1 (UBA1) encoded by the UBA1 gene, the NEDD8-activating enzyme E1 catalytic subunit protein (UBE1C) encoded by the UBA3 gene, the aquaporin 1 protein (AQP1) encoded by the AQP1 gene, the ubiquitin carboxyl-terminal esterase L1 protein (UCHL1) encoded by the UCHL1 gene, the ubiquitin carboxyl-terminal hydrolase isozyme L3 protein (UCHL3) encoded by the UCHL3 gene, the ubiquitin B protein (UBB) encoded by the UBB gene, the microtubule-associated protein tau (MAPT) encoded by the MAPT gene, the protein tyrosine phosphatase receptor type A protein (PTPRA) encoded by the PTPRA gene, the phosphatidylinositol binding clathrin assembly protein (PICALM) encoded by the PICALM gene, the clusterin protein (also known as apoplipoprotein J) encoded by the CLU gene, the presenilin 1 protein encoded by the PSEN1 gene, the presenilin 2 protein encoded by the PSEN2 gene, the sortilin-related receptor L (DLR class) A repeats-containing protein (SORL1) protein encoded by the SORL1 gene, the amyloid precursor protein (APP) encoded by the APP gene, the Apolipoprotein E precursor (APOE) encoded by the APOE gene, or the brain-derived neurotrophic factor (BDNF) encoded by the BDNF gene, or combinations thereof.

In certain embodiments, an animal created by a method of the invention may be used to study the effects of mutations on the animal and development and/or progression of AD using measures commonly used in the study of AD. Commonly used measures in the study of AD include without limit, learning and memory, anxiety, depression, addiction, and sensory-motor functions, as well as functional, pathological, metabolic, or biochemical assays. Those of skill in the art are familiar with other suitable measures or indicators of AD. In general, such measures may be made in comparison to wild type littermates.

Other measures of behavior may include assessments of spontaneous behavior. Spontaneous behavior may be assessed using any one or more methods of spontaneous behavioral observations known in the art. In general, any spontaneous behavior within a known behavioral repertoire of an animal may be observed, including movement, posture, social interaction, rearing, sleeping, blinking, eating, drinking, urinating, defecating, mating, and aggression. An extensive battery of observations for quantifying the spontaneous behavior of mice and rats is well-known in the art, including but not limited to home-cage observations such as body position, respiration, tonic involuntary movement, unusual motor behavior such as pacing or rocking, catatonic behavior, vocalization, palpebral closure, mating frequency, running wheel behavior, nest building, and frequency of aggressive interactions.

In another embodiment, the animals of the invention may be used to study the effects of the mutations on the progression of a disease state or disorder other than AD, but which is also associated with AD-related proteins, using measures commonly used in the study of said disease state or disorder. Non limiting examples of disease states or disorders other than AD that may be associated with AD-related proteins include dementia, congenital cerebellar ataxia, mental retardation such as learning and memory defects, lissencephaly, tauopathy or fibrilization, amyloidosis, neurodegeneration, Parkinsonism, progressive supranuclear palsy, Pick disease, male infertility, prostate and breast cancer, squamous cell carcinoma, lymphoma, leukemia, and atherosclerosis.

Yet another aspect encompasses a method for assessing the efficacy of a potential gene therapy strategy. That is, a chromosomal sequence encoding a protein associated with AD may be modified such that the genetically modified animal may have an altered response to the development and/or progression of AD as compared to a non treated animal. Stated another way, a mutated gene that predisposes an animal to AD may be “corrected” through gene therapy.

### F. Autism Spectrum Disorder

In one embodiment, a method of the invention may be used to create an animal or cell in which at least one chromosomal sequence associated with autism spectrum disorder (ASD) has been edited. Suitable chromosomal edits may include, but are not limited to, the type of edits detailed in section I(f) above.

In each of the above embodiments, one or more chromosomal sequences associated with ASD may be edited. An ASD associated protein or control sequence may typically be selected based on an experimental association of the protein or control sequence to an incidence or indication of an ASD. For example, the production rate or circulating concentration of a protein associated with ASD may be elevated or depressed in a population having an ASD relative to a population lacking the ASD. Differences in protein levels may be assessed using proteomic or genomic analysis techniques known in the art.

The identity of the protein associated with ASD whose chromosomal sequence is edited can and will vary. In preferred embodiments, the proteins associated with ASD whose chromosomal sequence is edited may be the benzodiazapine receptor (peripheral) associated protein 1 (BZRAP1) encoded by the BZRAP1 gene, the AF4/FMR2 family member 2 protein (AFF2) encoded by the AFF2 gene (also termed MFR2), the fragile X mental retardation autosomal homolog 1 protein (FXR1) encoded by the FXR1 gene, the fragile X mental retardation autosomal homolog 2 protein (FXR2) encoded by the FXR2 gene, the MAM domain containing glycosylphosphatidylinositol anchor 2 protein (MDGA2) encoded by the MDGA2 gene, the methyl CpG binding protein 2 (MECP2) encoded by the MECP2 gene, the metabotropic glutamate receptor 5 (MGLUR5) encoded by the MGLUR5-1 gene (also termed GRM5), the neurexin 1 protein encoded by the NRXN1 gene, or the semaphorin-5A protein (SEMA5A) encoded by the SEMA5A gene.

The edited or integrated chromosomal sequence may be modified to encode an altered protein associated with ASD. Non-limiting examples of mutations in proteins associated with ASD include the L18Q mutation in neurexin 1 where the leucine at position 18 is replaced with a glutamine, the R451C mutation in neuroligin 3 where the arginine at position 451 is replaced with a cysteine, the R87W mutation in neuroligin 4 where the arginine at position 87 is replaced with a tryptophan, and the 1425V mutation in serotonin transporter where the isoleucine at position 425 is replaced with a valine. A number of other mutations and chromosomal rearrangements in ASD-related chromosomal sequences have been associated with ASD and are known in the art. See, for example, Freitag et al. (2010) Eur. Child. Adolesc. Psychiatry 19:169-178, and Bucan et al. (2009) PLoS Genetics 5: e1000536, the disclosure of which is incorporated by reference herein in its entirety.

In certain embodiments, an animal created by a method of the invention may be used to study the effects of mutations on the animal and development and/or progression of ASD using measures commonly used in the study of ASD.

### G. Macular Degeneration

In one embodiment, a method of the invention may be used to create an animal or cell in which at least one chromosomal sequence associated with macular degeneration (MD) has been edited. Suitable chromosomal edits may include, but are not limited to, the type of edits detailed in section I(f) above.

In each of the above embodiments, one or more chromosomal sequences associated with MD may be edited. A MD-associated protein or control sequence may typically be selected based on an experimental association of the protein associated with MD to an MD disorder. For example, the production rate or circulating concentration of a protein associated with MD may be elevated or depressed in a population having an MD disorder relative to a population lacking the MD disorder. Differences in protein levels may be assessed using proteomic or genomic analysis techniques known in the art.

The identity of the protein associated with MD whose chromosomal sequence is edited can and will vary. In preferred embodiments, the proteins associated with MD whose chromosomal sequence is edited may be the ATP-binding cassette, sub-family A (ABC1) member 4 protein (ABCA4) encoded by the ABCR gene, the apolipoprotein E protein (APOE) encoded by the APOE gene, the chemokine (C-C motif) Ligand 2 protein (CCL2) encoded by the CCL2 gene, the chemokine (C-C motif) receptor 2 protein (CCR2) encoded by the CCR2 gene, the ceruloplasmin protein (CP) encoded by the CP gene, the cathepsin D protein (CTSD) encoded by the CTSD gene, or the metalloproteinase inhibitor 3 protein (TIMP3) encoded by the TIMP3 gene.

In certain embodiments, a genetically modified animal created by a method of the invention may be used to study the effects of mutations on the progression of MD using measures commonly used in the study of MD. Alternatively, the genetically modified animals of the invention may be used to study the effects of the mutations on the progression of a disease state or disorder associated with proteins associated with MD using measures commonly used in the study of said disease state or disorder. Non-limiting examples of measures that may be used include drusen accumulation, lipofuscin accumulation, thickening of Bruch's membrane, retinal degeneration, choroidal neovascularization, differential responses to a compound, abnormalities in tissues or cells, biochemical or molecular differences between genetically modified animals and wild type animals or a combination thereof.

### H. Schizophrenia

In one embodiment, a method of the invention may be used to create an animal or cell in which at least one chromosomal sequence associated with schizophrenia has been edited. Suitable chromosomal edits may include, but are not limited to, the type of edits detailed in section I(f) above.

In each of the above embodiments, one or more chromosomal sequences associated with schizophrenia may be edited. A schizophrenia-associated protein or control sequence may typically be selected based on an experimental association of the protein associated with schizophrenia to the development or progression of schizophrenia. For example, the production rate or circulating concentration of a protein associated with schizophrenia may be elevated or depressed in a population having schizophrenia relative to a population not having schizophrenia. Differences in protein levels may be assessed using proteomic or genomic analysis techniques known in the art. Exemplary non-limiting examples of chromosomal sequences associated with schizophrenia include NRG1, ErbB4, CPLX1, TPH1, TPH2, NRXN1, GSK3A, BDNF, DISC1, GSK3B, and combinations thereof, each of which is described in more detail below.

In certain embodiments, an animal created by a method of the invention may be used to study the effects of mutations on the animal and development and/or progression of MD using measures commonly used in the study of MD.

The incidence or indication of the schizophrenia or related disorder may occur spontaneously in the genetically modified animal. Alternatively, the incidence or indication of the schizophrenia or related disorder may be promoted by exposure to a disruptive agent. Non-limiting examples of disruptive agents include a protein associated with schizophrenia such as any of those described above, a drug, a toxin, a chemical, an activated retrovirus, and an environmental stress. Non-limiting examples of environmental stresses include forced swimming, cold swimming, platform shaker stimuli, loud noises, and immobilization stress.

### I. Tumor Suppression

Tumor suppression genes are genes whose protein products protect a cell from one step on the path to cancer. A mutation in a tumor suppressor gene may cause a loss or reduction in the protective function of its protein product, thereby increasing the probability that a tumor will form, leading to cancer, usually in combination with other genetic changes. The proteins encoded by tumor suppressor genes have a dampening or repressive effect on the regulation of the cell cycle or promote apoptosis, and sometimes both. Tumor suppressor proteins are involved in the repression of genes essential for the continuing cell cycle; coupling the cell cycle to DNA damage so that the cell cycle can continue; initiating apoptosis in the cell if the damage cannot be repaired; and cell adhesion to prevent tumors from dispersing, blocking a loss of contact inhibition, and inhibiting metastasis.

In one embodiment, a method of the invention may be used to create an animal or cell in which at least one chromosomal sequence associated with tumor suppression has been edited. Suitable chromosomal edits may include, but are not limited to, the type of edits detailed in section I(f) above.

In each of the above embodiments, one or more chromosomal sequences associated with tumor suppression may be edited. A tumor suppression-associated protein or control sequence may typically selected based on an experimental association of the protein of interest with a cancer. For example, the production rate or circulating concentration of a protein associated with tumor suppression may be elevated or depressed in a population having cancer relative to a population not having cancer. Differences in protein levels may be assessed using proteomic or genomic analysis techniques known in the art.

By way of example, proteins involved in tumor suppression may comprise, but are not limited to, TNF (tumor necrosis factor (TNF superfamily, member 2)), TP53 (tumor protein p53), ERBB2 (v-erb-b2 erythroblastic leukemia viral oncogene homolog 2, neuro/glioblastoma derived oncogene homolog (avian)), FN1 (fibronectin 1), TSC1 (tuberous sclerosis 1), PTGS2 (prostaglandin-endoperoxide synthase 2 (prostaglandin G/H synthase and cyclooxygenase)), PTEN (phosphatase and tensin homolog), PCNA (proliferating cell nuclear antigen), COL18A1 (collagen, type XVIII, alpha 1), TSSC4 (tumor suppressing subtransferable candidate 4), JUN (jun oncogene), MAPK8 (mitogen-activated protein kinase 8), TGFB1 (transforming growth factor, beta 1), IL6 (interleukin 6 (interferon, beta 2)), IFNG (interferon, gamma), BRCA1 (breast cancer 1, early onset), TSPAN32 (tetraspanin 32), BCL2 (B-cell CLL/lymphoma 2), NF2 (neurofibromin 2 (merlin)), GJB1 (gap junction protein, beta 1, 32 kDa), MAPK1 (mitogen-activated protein kinase 1), CD44 (CD44 molecule (Indian blood group)), PGR (progesterone receptor), TNS1 (tensin 1), PROK1 (prokineticin 1), SIAH1 (seven in absentia homolog 1 (Drosophila)), ENG (endoglin), TP73 (tumor protein p73), APC (adenomatous polyposis coli), BAX (BCL2-associated X protein), SRC (v-src sarcoma (Schmidt-Ruppin A-2) viral oncogene homolog (avian)), VHL (von Hippel-Lindau tumor suppressor), FHIT (fragile histidine triad gene), NFKB1 (nuclear factor of kappa light polypeptide gene enhancer in B-cells 1), IFNα1 (interferon, alpha 1), TGFBR1 (transforming growth factor, beta receptor 1), PRKCD (protein kinase C, delta), TGIF1 (TGFB-induced factor homeobox 1), DLC1 (deleted in liver cancer 1), SLC22A18 (solute carrier family 22, member 18), VEGFA (vascular endothelial growth factor A), MME (membrane metallo-endopeptidase), IL3 (interleukin 3 (colony-stimulating factor, multiple)), MKI67 (antigen identified by monoclonal antibody Ki-67), HSPD1 (heat shock 60 kDa protein 1 (chaperonin)), HSPB1 (heat shock 27 kDa protein 1), HSP90B2P (heat shock protein 90 kDa beta (Grp94), member 2 (pseudogene)), MBL2 (mannose-binding lectin (protein C) 2, soluble (opsonic defect)), ZFYVE9 (zinc finger, FYVE domain containing 9), TERT (telomerase reverse transcriptase), PML (promyelocytic leukemia), SKP2 (S-phase kinase-associated protein 2 (p45)), CYCS (cytochrome c, somatic), MAPK10 (mitogen-activated protein kinase 10), PAX7 (paired box 7), YAP1 (Yes-associated protein 1), PARP1 (poly (ADP-ribose) polymerase 1), MIR34A (microRNA 34a), PRKCA (protein kinase C, alpha), FAS (Fas (TNF receptor superfamily, member 6)), SYK (spleen tyrosine kinase), GSK3B (glycogen synthase kinase 3 beta), PRKCE (protein kinase C, epsilon), CYP19A1 (cytochrome P450, family 19, subfamily A, polypeptide 1), ABCB1 (ATP-binding cassette, sub-family B (MDR/TAP), member 1), NFKBIA (nuclear factor of kappa light polypeptide gene enhancer in B-cells inhibitor, alpha), RUNX1 (runt-related transcription factor 1), PRKCG (protein kinase C, gamma), RELA (v-rel reticuloendotheliosis viral oncogene homolog A (avian)), PLAU (plasminogen activator, urokinase), BTK (Bruton agammaglobulinemia tyrosine kinase), PRKCB (protein kinase C, beta), CSF1 (colony stimulating factor 1 (macrophage)), POMC (proopiomelanocortin), CEBPB (CCAAT/enhancer binding protein (C/EBP), beta), ROCK1 (Rho-associated, coiled-coil containing protein kinase 1), KDR (kinase insert domain receptor (a type III receptor tyrosine kinase)), NPM1 (nucleophosmin (nucleolar phosphoprotein B23, numatrin)), ROCK2 (Rho-associated, coiled-coil containing protein kinase 2), PRKAB1 (protein kinase, AMP-activated, beta 1 non-catalytic subunit), BAK1 (BCL2-antagonist/killer 1), AURKA (aurora kinase A), NTN1 (netrin 1), FLT1 (fms-related tyrosine kinase 1 (vascular endothelial growth factor/vascular permeability factor receptor)), NBN (nibrin), DNM3 (dynamin 3), PRDM10 (PR domain containing 10), PAX5 (paired box 5), EIF4G1 (eukaryotic translation initiation factor 4 gamma, 1), KAT2B (K(lysine) acetyltransferase 2B), TIMP3 (TIMP metallopeptidase inhibitor 3), CCL22 (chemokine (C-C motif) ligand 22), GRIN2B (glutamate receptor, ionotropic, N-methyl D-aspartate 2B), CD81 (CD81 molecule), CCL27 (chemokine (C-C motif) ligand 27), MAPK11 (mitogen-activated protein kinase 11), DKK1 (dickkopf homolog 1 (Xenopus laevis)), HYAL1 (hyaluronoglucosaminidase 1), CTSL1 (cathepsin L1), PKD1 (polycystic kidney disease 1 (autosomal dominant)), BUB1B (budding uninhibited by benzimidazoles 1 homolog beta (yeast)), MPP1 (membrane protein, palmitoylated 1, 55 kDa), SIAH2 (seven in absentia homolog 2 (Drosophila)), DUSP13 (dual specificity phosphatase 13), CCL21 (chemokine (C-C motif) ligand 21), RTN4 (reticulon 4), SMO (smoothened homolog (Drosophila)), CCL19 (chemokine (C-C motif) ligand 19), CSTF2 (cleavage stimulation factor, 3\′ pre-RNA, subunit 2, 64 kDa), RSF1 (remodeling and spacing factor 1), EZH2 (enhancer of zeste homolog 2 (Drosophila)), AK1 (adenylate kinase 1), CKM (creatine kinase, muscle), HYAL3 (hyaluronoglucosaminidase 3), ALOX15B (arachidonate 15-lipoxygenase, type B), PAG1 (phosphoprotein associated with glycosphingolipid microdomains 1), MIR21 (microRNA 21), S100A2 (S100 calcium binding protein A2), HYAL2 (hyaluronoglucosaminidase 2), CSTF1 (cleavage stimulation factor, 3V pre-RNA, subunit 1, 50 kDa), PCGF2 (polycomb group ring finger 2), THSD1 (thrombospondin, type I, domain containing 1), HOPX (HOP homeobox), SLC5A8 (solute carrier family 5 (iodide transporter), member 8), EMB (embigin homolog (mouse)), PAX9 (paired box 9), ARMCX3 (armadillo repeat containing, X-linked 3), ARMCX2 (armadillo repeat containing, X-linked 2), ARMCX1 (armadillo repeat containing, X-linked 1), RASSF4 (Ras association (RalGDS/AF-6) domain family member 4), MIR34B (microRNA 34b), MIR205 (microRNA 205), RB1 (retinoblastoma 1), DYT10 (dystonia 10), CDKN2A (cyclin-dependent kinase inhibitor 2A (melanoma, p16, inhibits CDK4)), CDKN1A (cyclin-dependent kinase inhibitor 1A (p21, Cip1)), CCND1 (cyclin D1), AKT1 (v-akt murine thymoma viral oncogene homolog 1), MYC (v-myc myelocytomatosis viral oncogene homolog (avian)), CTNNB1 (catenin (cadherin-associated protein), beta 1, 88 kDa), MDM2 (Mdm2 p53 binding protein homolog (mouse)), SERPINB5 (serpin peptidase inhibitor, clade B (ovalbumin), member 5), EGF (epidermal growth factor (beta-urogastrone)), FOS (FBJ murine osteosarcoma viral oncogene homolog), NOS2 (nitric oxide synthase 2, inducible), CDK4 (cyclin-dependent kinase 4), SOD2 (superoxide dismutase 2, mitochondrial), SMAD3 (SMAD family member 3), CDKN1B (cyclin-dependent kinase inhibitor 1B (p27, Kip1)), SOD1 (superoxide dismutase 1, soluble), CCNA2 (cyclin A2), LOX (lysyl oxidase), SMAD4 (SMAD family member 4), HGF (hepatocyte growth factor (hepapoietin A; scatter factor)), THBS1 (thrombospondin 1), CDK6 (cyclin-dependent kinase 6), ATM (ataxia telangiectasia mutated), STAT3 (signal transducer and activator of transcription 3 (acute-phase response factor)), HIF1A (hypoxia inducible factor 1, alpha subunit (basic helix-loop-helix transcription factor)), IGF1R (insulin-like growth factor 1 receptor), MTOR (mechanistic target of rapamycin (serine/threonine kinase)), TSC2 (tuberous sclerosis 2), CDC42 (cell division cycle 42 (GTP binding protein, 25 kDa)), ODC1 (ornithine decarboxylase 1), SPARC (secreted protein, acidic, cysteine-rich (osteonectin)), HDAC1 (histone deacetylase 1), CDK2 (cyclin-dependent kinase 2), BARD1 (BRCA1 associated RING domain 1), CDH1 (cadherin 1, type 1, E-cadherin (epithelial)), EGR1 (early growth response 1), INSR (insulin receptor), IRF1 (interferon regulatory factor 1), PHB (prohibitin), PXN (paxillin), HSPA4 (heat shock 70 kDa protein 4), TYR (tyrosinase (oculocutaneous albinism IA)), CAV1 (caveolin 1, caveolae protein, 22 kDa), CDKN2B (cyclin-dependent kinase inhibitor 2B (p15, inhibits CDK4)), FOXO3 (forkhead box O3), HDAC9 (histone deacetylase 9), FBXW7 (F-box and WD repeat domain containing 7), FOXO1 (forkhead box 01), E2F1 (E2F transcription factor 1), STK11 (serine/threonine kinase 11), BMP2 (bone morphogenetic protein 2), HSP90AA1 (heat shock protein 90 kDa alpha (cytosolic), class A member 1), HNF4A (hepatocyte nuclear factor 4, alpha), CAMK2G (calcium/calmodulin-dependent protein kinase II gamma), TP53BP1 (tumor protein p53 binding protein 1), CRYAB (crystallin, alpha B), HMGCR (3-hydroxy-3-methylglutaryl-Coenzyme A reductase), PLAUR (plasminogen activator, urokinase receptor), MCL1 (myeloid cell leukemia sequence 1 (BCL2-related)), NOTCH1 (Notch homolog 1, translocation-associated (Drosophila)), RASSF1 (Ras association (RalGDS/AF-6) domain family member 1), GSN (gelsolin), CADM1 (cell adhesion molecule 1), ATF2 (activating transcription factor 2), IFNB1 (interferon, beta 1, fibroblast), DAPK1 (death-associated protein kinase 1), CHFR (checkpoint with forkhead and ring finger domains), KITLG (KIT ligand), NDUFA13 (NADH dehydrogenase (ubiquinone) 1 alpha subcomplex, 13), DPP4 (dipeptidyl-peptidase 4), GLB1 (galactosidase, beta 1), IKZF1 (IKAROS family zinc finger 1 (Ikaros)), ST5 (suppression of tumorigenicity 5), TGFA (transforming growth factor, alpha), EIF4EBP1 (eukaryotic translation initiation factor 4E binding protein 1), TGFBR2 (transforming growth factor, beta receptor II (70/80 kDa)), EIF2AK2 (eukaryotic translation initiation factor 2-alpha kinase 2), GJA1 (gap junction protein, alpha 1, 43 kDa), MYD88 (myeloid differentiation primary response gene (88)), IF127 (interferon, alpha-inducible protein 27), RBMX (RNA binding motif protein, X-linked), EPHA1 (EPH receptor A1), TWSG1 (twisted gastrulation homolog 1 (Drosophila)), H2AFX (H2A histone family, member X), LGALS3 (lectin, galactoside-binding, soluble, 3), MUC3A (mucin 3A, cell surface associated), ILK (integrin-linked kinase), APAF1 (apoptotic peptidase activating factor 1), MAOA (monoamine oxidase A), ERBB3 (v-erb-b2 erythroblastic leukemia viral oncogene homolog 3 (avian)), EIF2S1 (eukaryotic translation initiation factor 2, subunit 1 alpha, 35 kDa), PER2 (period homolog 2 (Drosophila)), IGFBP7 (insulin-like growth factor binding protein 7), KDM5B (lysine (K)-specific demethylase 5B), SMARCA4 (SWI/SNF related, matrix associated, actin dependent regulator of chromatin, subfamily a, member 4), NME1 (non-metastatic cells 1, protein (NM23A) expressed in), F2RL1 (coagulation factor II (thrombin) receptor-like 1), ZFP36 (zinc finger protein 36, C3H type, homolog (mouse)), HSPA8 (heat shock 70 kDa protein 8), WNT5A (wingless-type MMTV integration site family, member 5A), ITGB4 (integrin, beta 4), RARB (retinoic acid receptor, beta), VEGFC (vascular endothelial growth factor C), CCL20 (chemokine (C-C motif) ligand 20), EPHB2 (EPH receptor B2), CSNK2A1 (casein kinase 2, alpha 1 polypeptide), PSMD9 (proteasome (prosome, macropain) 26S subunit, non-ATPase, 9), SERPINB2 (serpin peptidase inhibitor, clade B (ovalbumin), member 2), RHOB (ras homolog gene family, member B), DUSP6 (dual specificity phosphatase 6), CDKN1C (cyclin-dependent kinase inhibitor 1C (p57, Kip2)), SLIT2 (slit homolog 2 (Drosophila)), CEACAM1 (carcinoembryonic antigen-related cell adhesion molecule 1 (biliary glycoprotein)), UBC (ubiquitin C), STS (steroid sulfatase (microsomal), isozyme S), FST (follistatin), KRT1 (keratin 1), EIF6 (eukaryotic translation initiation factor 6), JUP (junction plakoglobin), HDAC4 (histone deacetylase 4), NEDD4 (neural precursor cell expressed, developmentally down-regulated 4), KRT14 (keratin 14), GLI2 (GLI family zinc finger 2), MYH11 (myosin, heavy chain 11, smooth muscle), MAPKAPK5 (mitogen-activated protein kinase-activated protein kinase 5), MAD1L1 (MAD1 mitotic arrest deficient-like 1 (yeast)), TNFAIP3 (tumor necrosis factor, alpha-induced protein 3), WEE1 (WEE1 homolog (S. pombe)), BTRC (beta-transducin repeat containing), NKX3-1 (NK3 homeobox 1), GPC3 (glypican 3), CREB3 (cAMP responsive element binding protein 3), PLCB3 (phospholipase C, beta 3 (phosphatidylinositol-specific)), DMPK (dystrophia myotonica-protein kinase), BLNK (B-cell linker), PPIA (peptidylprolyl isomerase A (cyclophilin A)), DAB2 (disabled homolog 2, mitogen-responsive phosphoprotein (Drosophila)), KLF4 (Kruppel-like factor 4 (gut)), RUNX3 (runt-related transcription factor 3), FLG (filaggrin), IVL (involucrin), CCT5 (chaperonin containing TCP1, subunit 5 (epsilon)), LRPAP1 (low density lipoprotein receptor-related protein associated protein 1), IGF2R (insulin-like growth factor 2 receptor), PER1 (period homolog 1 (Drosophila)), BIK (BCL2-interacting killer (apoptosis-inducing)), PSMC4 (proteasome (prosome, macropain) 26S subunit, ATPase, 4), USF2 (upstream transcription factor 2, c-fos interacting), GAS1 (growth arrest-specific 1), LAMP2 (lysosomal-associated membrane protein 2), PSMD10 (proteasome (prosome, macropain) 26S subunit, non-ATPase, 10), IL24 (interleukin 24), GADD45G (growth arrest and DNA-damage-inducible, gamma), ARHGAP1 (Rho GTPase activating protein 1), CLDN1 (claudin 1), ANXA7 (annexin A7), CHN1 (chimerin (chimaerin) 1), TXNIP (thioredoxin interacting protein), PEG3 (paternally expressed 3), EIF3A (eukaryotic translation initiation factor 3, subunit A), CASC5 (cancer susceptibility candidate 5), TCF4 (transcription factor 4), CSNK2A2 (casein kinase 2, alpha prime polypeptide), CSNK2B (casein kinase 2, beta polypeptide), CRY1 (cryptochrome 1 (photolyase-like)), CRY2 (cryptochrome 2 (photolyase-like)), EIF4G2 (eukaryotic translation initiation factor 4 gamma, 2), LOXL2 (lysyl oxidase-like 2), PSMD13 (proteasome (prosome, macropain) 26S subunit, non-ATPase, 13), ANP32A (acidic (leucine-rich) nuclear phosphoprotein 32 family, member A), COL4A3 (collagen, type IV, alpha 3 (Goodpasture antigen)), SCGB1A1 (secretoglobin, family 1A, member 1 (uteroglobin)), BNIP3L (BCL2/adenovirus E1B 19 kDa interacting protein 3-like), MCC (mutated in colorectal cancers), EFNB3 (ephrin-B3), RBBP8 (retinoblastoma binding protein 8), PALB2 (partner and localizer of BRCA2), HBP1 (HMG-box transcription factor 1), MRPL28 (mitochondrial ribosomal protein L28), KDM5A (lysine (K)-specific demethylase 5A), QSOX1 (quiescin Q6 sulfhydryl oxidase 1), ZFR (zinc finger RNA binding protein), MN1 (meningioma (disrupted in balanced translocation) 1), SMYD4 (SET and MYND domain containing 4), USP7 (ubiquitin specific peptidase 7 (herpes virus-associated)), STK4 (serine/threonine kinase 4), THY1 (Thy-1 cell surface antigen), PTPRG (protein tyrosine phosphatase, receptor type, G), E2F6 (E2F transcription factor 6), STX11 (syntaxin 11), CDC42BPA (CDC42 binding protein kinase alpha (DMPK-like)), MYOCD (myocardin), DAP (death-associated protein), LOXL1 (lysyl oxidase-like 1), RNF139 (ring finger protein 139), HTATIP2 (HIV-1 Tat interactive protein 2, 30 kDa), AIM1 (absent in melanoma 1), BCClP (BRCA2 and CDKN1A interacting protein), LOXL4 (lysyl oxidase-like 4), WWC1 (WW and C2 domain containing 1), LOXL3 (lysyl oxidase-like 3), CENPN (centromere protein N), TNS4 (tensin 4), SIK1 (salt-inducible kinase 1), PCGF6 (polycomb group ring finger 6), PHLDA3 (pleckstrin homology-like domain, family A, member 3), IL32 (interleukin 32), LATS1 (LATS, large tumor suppressor, homolog 1 (Drosophila)), COMMD7 (COMM domain containing 7), CDHR2 (cadherin-related family member 2), LELP1 (late cornified envelope-like proline-rich 1), NCRNA00188 (non-protein coding RNA 188), and ENSG00000131023.

Exemplary non-limiting examples of tumor suppression proteins include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, Notch 4, ATK1 (v-akt murine thymoma viral oncogene homolog 1), ATK2 (v-akt murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R(fibroblast growth factor 1 receptor), FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia)), VHL (von-Hippel-Lindau tumor suppressor), BRCA1 (breast cancer 1), BRCA2 (breast cancer 2), AR (androgen receptor), TSG101 (tumor susceptibility gene 101), Igf1 (insulin-like growth factor 1), Igf2 (insulin-like growth factor 2), Igf 1R (insulin-like growth factor 1 receptor), Igf 2R (insulin-like growth factor 2 receptor) Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7(Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), p53, and combinations thereof.

In certain embodiments, an animal created by a method of the invention may be used to study the effects of mutations on the animal and on tumor suppression using measures commonly used in the study of tumor suppression. In one embodiment, a genetically modified animal comprising an inactivated chromosomal sequence involved with tumor suppression may be used to determine susceptibility to developing tumors. The method comprises exposing the genetically modified animal comprising an inactivated tumor suppressor sequence and a wild-type animal to a carcinogenic agent, and then monitoring the development of tumors. The animal comprising the inactivated tumor suppressor sequence may have an increased risk for tumor formation. Moreover, an animal homozygous for the inactivated tumor suppressor sequence may have increased risk relative to an animal heterozygous for the same inactivated sequence, which in turn may have increased risk relative to a wild-type animal. A similar method may be used to screen for spontaneous tumors, wherein the animals described above are not exposed to a carcinogenic agent.

In another embodiment, an animal comprising an inactivated chromosomal sequence associated with tumor suppression may be used to evaluate the carcinogenic potential of a test agent. The method comprises contacting the genetically modified animal comprising an inactivated tumor suppressor sequence and a wild-type animal to the test agent, and then monitoring the development of tumors. If the animal comprising an inactivated tumor suppressor sequence has an increased incidence of tumors relative to the wild-type animal, the test agent may be carcinogenic.

### J. Secretase Associated Disorders

Secretases make up a diverse set of proteins that affect susceptibility for numerous disorders, the presence of a disorder, the severity of a disorder, or any combination thereof. Secretases are enzymes that clip off smaller pieces of another transmembrane protein. Secretases are implicated in many disorders including, for example, Alzheimer's discase. In one embodiment, a method of the invention may be used to create an animal or cell in which at least one chromosomal sequence associated with secretase associated disorders has been edited. Suitable chromosomal edits may include, but are not limited to, the type of edits detailed in section I(f) above.

In each of the above embodiments, one or more chromosomal sequences associated with a secretase associated disorder may be edited. A secretase associated disorder-associated protein or control sequence may typically be selected based on an experimental association of the secretase-related proteins with the development of a secretase disorder. For example, the production rate or circulating concentration of a protein associated with a secretase disorder may be elevated or depressed in a population with a secretase disorder relative to a population without a secretase disorder. Differences in protein levels may be assessed using proteomic or genomic analysis techniques known in the art.

By way of non-limiting example, proteins associated with a secretase disorder include PSENEN (presenilin enhancer 2 homolog (C. elegans)), CTSB (cathepsin B), PSEN1 (presenilin 1), APP (amyloid beta (A4) precursor protein), APH1B (anterior pharynx defective 1 homolog B (C. elegans)), PSEN2 (presenilin 2 (Alzheimer disease 4)), BACE1 (beta-site APP-cleaving enzyme 1), ITM2B (integral membrane protein 2B), CTSD (cathepsin D), NOTCH1 (Notch homolog 1, translocation-associated (Drosophila)), TNF (tumor necrosis factor (TNF superfamily, member 2)), INS (insulin), DYT10 (dystonia 10), ADAM17 (ADAM metallopeptidase domain 17), APOE (apolipoprotein E), ACE (angiotensin I converting enzyme (peptidyl-dipeptidase A) 1), STN (statin), TP53 (tumor protein p53), IL6 (interleukin 6 (interferon, beta 2)), NGFR (nerve growth factor receptor (TNFR superfamily, member 16)), IL1B (interleukin 1, beta), ACHE (acetylcholinesterase (Yt blood group)), CTNNB1 (catenin (cadherin-associated protein), beta 1, 88 kDa), IGF1 (insulin-like growth factor 1 (somatomedin C)), IFNG (interferon, gamma), NRG1 (neuregulin 1), CASP3 (caspase 3, apoptosis-related cysteine peptidase), MAPK1 (mitogen-activated protein kinase 1), CDH1 (cadherin 1, type 1, E-cadherin (epithelial)), APBB1 (amyloid beta (A4) precursor protein-binding, family B, member 1 (Fe65)), HMGCR (3-hydroxy-3-methylglutaryl-Coenzyme A reductase), CREB1 (cAMP responsive element binding protein 1), PTGS2 (prostaglandin-endoperoxide synthase 2 (prostaglandin G/H synthase and cyclooxygenase)), HES1 (hairy and enhancer of split 1, (Drosophila)), CAT (catalase), TGFB1 (transforming growth factor, beta 1), ENO2 (enolase 2 (gamma, neuronal)), ERBB4 (v-erb-a erythroblastic leukemia viral oncogene homolog 4 (avian)), TRAPPC10 (trafficking protein particle complex 10), MAOB (monoamine oxidase B), NGF (nerve growth factor (beta polypeptide)), MMP12 (matrix metallopeptidase 12 (macrophage elastase)), JAG1 (jagged 1 (Alagille syndrome)), CD40LG (CD40 ligand), PPARG (peroxisome proliferator-activated receptor gamma), FGF2 (fibroblast growth factor 2 (basic)), IL3 (interleukin 3 (colony-stimulating factor, multiple)), LRP1 (low density lipoprotein receptor-related protein 1), NOTCH4 (Notch homolog 4 (Drosophila)), MAPK8 (mitogen-activated protein kinase 8), PREP (prolyl endopeptidase), NOTCH3 (Notch homolog 3 (Drosophila)), PRNP (prion protein), CTSG (cathepsin G), EGF (epidermal growth factor (beta-urogastrone)), REN (renin), CD44 (CD44 molecule (Indian blood group)), SELP (selectin P (granule membrane protein 140 kDa, antigen CD62)), GHR (growth hormone receptor), ADCYAP1 (adenylate cyclase activating polypeptide 1 (pituitary)), INSR (insulin receptor), GFAP (glial fibrillary acidic protein), MMP3 (matrix metallopeptidase 3 (stromelysin 1, progelatinase)), MAPK10 (mitogen-activated protein kinase 10), SP1 (Sp1 transcription factor), MYC (v-myc myelocytomatosis viral oncogene homolog (avian)), CTSE (cathepsin E), PPARA (peroxisome proliferator-activated receptor alpha), JUN (jun oncogene), TIMP1 (TIMP metallopeptidase inhibitor 1), IL5 (interleukin 5 (colony-stimulating factor, eosinophil)), ILIA (interleukin 1, alpha), MMP9 (matrix metallopeptidase 9 (gelatinase B, 92 kDa gelatinase, 92 kDa type IV collagenase)), HTR4 (5-hydroxytryptamine (serotonin) receptor 4), HSPG2 (heparan sulfate proteoglycan 2), KRAS (v-Ki-ras2 Kirsten rat sarcoma viral oncogene homolog), CYCS (cytochrome c, somatic), SMG1 (SMG1 homolog, phosphatidylinositol 3-kinase-related kinase (C. elegans)), IL1RI (interleukin 1 receptor, type I), PROK1 (prokineticin 1), MAPK3 (mitogen-activated protein kinase 3), NTRK1 (neurotrophic tyrosine kinase, receptor, type 1), IL13 (interleukin 13), MME (membrane metallo-endopeptidase), TKT (transketolase), CXCR2 (chemokine (C-X-C motif) receptor 2), IGF1R (insulin-like growth factor 1 receptor), RARA (retinoic acid receptor, alpha), CREBBP (CREB binding protein), PTGS1 (prostaglandin-endoperoxide synthase 1 (prostaglandin G/H synthase and cyclooxygenase)), GALT (galactose-1-phosphate uridylyltransferase), CHRM1 (cholinergic receptor, muscarinic 1), ATXN1 (ataxin 1), PAWR (PRKC, apoptosis, WT1, regulator), NOTCH2 (Notch homolog 2 (Drosophila)), M6PR (mannose-6-phosphate receptor (cation dependent)), CYP46A1 (cytochrome P450, family 46, subfamily A, polypeptide 1), CSNK1D (casein kinase 1, delta), MAPK14 (mitogen-activated protein kinase 14), PRG2 (proteoglycan 2, bone marrow (natural killer cell activator, eosinophil granule major basic protein)), PRKCA (protein kinase C, alpha), L1CAM (L1 cell adhesion molecule), CD40 (CD40 molecule, TNF receptor superfamily member 5), NR112 (nuclear receptor subfamily 1, group I, member 2), JAG2 (jagged 2), CTNND1 (catenin (cadherin-associated protein), delta 1), CDH2 (cadherin 2, type 1, N-cadherin (neuronal)), CMA1 (chymase 1, mast cell), SORT1 (sortilin 1), DLK1 (delta-like 1 homolog (Drosophila)), THEM4 (thioesterase superfamily member 4), JUP (junction plakoglobin), CD46 (CD46 molecule, complement regulatory protein), CCL11 (chemokine (C-C motif) ligand 11), CAV3 (caveolin 3), RNASE3 (ribonuclease, RNase A family, 3 (eosinophil cationic protein)), HSPA8 (heat shock 70 kDa protein 8), CASP9 (caspase 9, apoptosis-related cysteine peptidase), CYP3A4 (cytochrome P450, family 3, subfamily A, polypeptide 4), CCR3 (chemokine (C-C motif) receptor 3), TFAP2A (transcription factor AP-2 alpha (activating enhancer binding protein 2 alpha)), SCP2 (sterol carrier protein 2), CDK4 (cyclin-dependent kinase 4), HIF1A (hypoxia inducible factor 1, alpha subunit (basic helix-loop-helix transcription factor)), TCF7L2 (transcription factor 7-like 2 (T-cell specific, HMG-box)), IL1R2 (interleukin 1 receptor, type II), B3GALTL (beta 1,3-galactosyltransferase-like), MDM2 (Mdm2 p53 binding protein homolog (mouse)), RELA (v-rel reticuloendotheliosis viral oncogene homolog A (avian)), CASP7 (caspase 7, apoptosis-related cysteine peptidase), IDE (insulin-degrading enzyme), FABP4 (fatty acid binding protein 4, adipocyte), CASK (calcium/calmodulin-dependent serine protein kinase (MAGUK family)), ADCYAP1R1 (adenylate cyclase activating polypeptide 1 (pituitary) receptor type I), ATF4 (activating transcription factor 4 (tax-responsive enhancer element B67)), PDGFA (platelet-derived growth factor alpha polypeptide), C21orf33 (chromosome 21 open reading frame 33), SCG5 (secretogranin V (7B2 protein)), RNF123 (ring finger protein 123), NFKB1 (nuclear factor of kappa light polypeptide gene enhancer in B-cells 1), ERBB2 (v-erb-b2 erythroblastic leukemia viral oncogene homolog 2, neuro/glioblastoma derived oncogene homolog (avian)), CAV1 (caveolin 1, caveolae protein, 22 kDa), MMP7 (matrix metallopeptidase 7 (matrilysin, uterine)), TGFα (transforming growth factor, alpha), RXRA (retinoid X receptor, alpha), STX1A (syntaxin 1A (brain)), PSMC4 (proteasome (prosome, macropain) 26S subunit, ATPase, 4), P2RY2 (purinergic receptor P2Y, G-protein coupled, 2), TNFRSF21 (tumor necrosis factor receptor superfamily, member 21), DLG1 (discs, large homolog 1 (Drosophila)), NUMBL (numb homolog (Drosophila)-like), SPN (sialophorin), PLSCR1 (phospholipid scramblase 1), UBQLN2 (ubiquilin 2), UBQLN1 (ubiquilin 1), PCSK7 (proprotein convertase subtilisin/kexin type 7), SPON1 (spondin 1, extracellular matrix protein), SILV (silver homolog (mouse)), QPCT (glutaminyl-peptide cyclotransferase), HES5 (hairy and enhancer of split 5 (Drosophila)), GCC1 (GRIP and coiled-coil domain containing 1), and any combination thereof.

Preferred proteins associated with a secretase disorder include APH-1A (anterior pharynx-defective 1, alpha), APH-1B (anterior pharynx-defective 1, beta), PSEN-1 (presenilin-1), NCSTN (nicastrin), PEN-2 (presenilin enhancer 2), and any combination thereof.

In certain embodiments, an animal created by a method of the invention may be used to study the effects of mutations on the animal and development and/or progression of a secretase associated disorder using measures commonly used in the study of secretase disorders.

The incidence or indication of a secretase disorder may occur spontaneously in the genetically modified animal. Alternatively, the incidence or indication of the secretase disorder may be promoted by exposure to a disruptive agent. Non-limiting examples of disruptive agents include a protein associated with a secretase disorder such as any of those described above, a drug, a toxin, a chemical, an activated retrovirus, and an environmental stress. Non-limiting examples of environmental stresses include forced swimming, cold swimming, platform shaker stimuli, loud noises, and immobilization stress.

### K. Amyotrophic Lateral Sclerosis

Certain nucleic acid sequences, and the proteins encoded by them, are associated with motor neuron disorders. These sequences make up a diverse set of sequences that affect susceptibility for developing a motor neuron disorder, the presence of the motor neuron disorder, the severity of the motor neuron disorder or any combination thereof. In one embodiment, a method of the invention may be used to create an animal or cell in which at least one chromosomal sequence associated with a specific motor neuron disorder, amyotrophic lateral sclerosis (ALS), has been edited. Suitable chromosomal edits may include, but are not limited to, the type of edits detailed in section I(f) above.

In each of the above embodiments, one or more chromosomal sequences associated with ALS may be edited. A chromosomal sequence associated with ALS may typically be selected based on an experimental association of an ALS-related sequence to ALS. An ALS-related nucleic acid sequence may encode an ALS-related protein or may be an ALS-related control sequence. For example, the production rate or circulating concentration of a protein associated with ALS may be elevated or depressed in a population with ALS relative to a population without ALS. Differences in protein levels may be assessed using proteomic or genomic analysis techniques known in the art.

By way of non-limiting example, proteins associated with ALS include but are not limited to SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

In certain embodiments, an animal created by a method of the invention may be used to study the effects of mutations on the animal and development and/or progression of ALS using measures commonly used in the study of ALS.

### L. Prion Diseases

Prion disorders appear to be diseases of protein conformation, which results in abnormal protein aggregation. In one embodiment, a method of the invention may be used to create an animal or cell in which at least one chromosomal sequence associated with a prion disease has been edited. Suitable chromosomal edits may include, but are not limited to, the type of edits detailed in section I(f) above.

In each of the above embodiments, one or more chromosomal sequences encoding a protein or control sequence associated with prion disorders may be edited. A prion disorder-related nucleic acid sequence may typically be selected based on an experimental association of the prion disorder-related nucleic acid sequence to a prion disorder. A prion disorder-related nucleic acid sequence may encode a prion disorder-related protein or isoform thereof, or may be a prion disorder-related control sequence. For example, the production rate or circulating concentration of a prion disorder-related protein or isoform may be elevated or depressed in a population having a prion disorder relative to a population lacking the prion disorder. Differences in protein or certain isoform levels may be assessed using proteomic techniques including but not limited to Western blot, immunohistochemical staining, enzyme linked immunosorbent assay (ELISA), and mass spectrometry. Alternatively, the prion disorder-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

Non-limiting examples of prion disorder-related proteins include PrP^{C} and its isoforms, PrP^{Sc} and its isoforms, HECTD2 (e3-ubipuitin ligase protein), STI1 (stress inducible protein 1), DPL (residue Doppel protein, encoded by Prnd), APOA1 (Apolipoprotein A1), BCL-2 (B-cell lymphoma 2), HSP60 (Heat shock 60 kDa protein), BAX-inhibiting peptide (Bcl-2-associated X protein inhibitor), NRF2 (nuclear respiratory factor 2), NCAMs (neural cell-adhesion molecules), heparin, laminin and laminin receptor.

Further, non-limiting examples of genes that may be related to neurodegenerative conditions in prion disorders include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), ADRA1D (Alpha-1D adrenergic receptor for Alpha-1D adrenoreceptor), AHSG (Alpha-2-HS-glycoprotein), A1F1 (Allograft inflammatory factor 1), ALAS2 (Delta-aminolevulinate synthase 2), AMBP (Alpha-1-microglobulin/bikunin precursor), ANK3 (Ankryn 3), ANXA3 (Annexin A3), APCS (Amyloid P component serum), APOA1 (Apolipoprotein A1), APOA12 (Apolipoprotein A2), APOB (Apolipoprotein B), APOCl (Apolipoprotein C1), APOE (Apolipoprotein E), APOH (Apolipoprotein H), APP (Amyloid precursor protein), ARC (Activity-regulated cytoskeleton-associated protein), ARF6 (ADP-ribosylation factor 6), ARHGAP5 (Rho GTPase activating protein 5), ASCL1 (Achaete-scute homolog 1), B2M (Beta-2 microglobulin), B4GALNT1 (Beta-1,4-N-acetyl-galactosaminyl transferase 1), BAX (BcI-2-associated X protein), BCAT (Branched chain amino-acid transaminase 1 cytosolic), BCKDHA (Branched chain keto acid dehydrogenase E1 alpha), BCKDK (Branched chain alpha-ketoacid dehydrogenase kinase), BCL2 (B-cell lymphoma 2), BCL2L1 (BCL2-like 1), BDNF (Brain-derived neurotrophic factor), BHLHE40 (Class E basic helix-loop-helix protein 40), BHLHE41 (Class E basic helix-loop-helix protein 41), BMP2 (Bone morphogenetic protein 2A), BMP3 (Bone morphogenetic protein 3), BMP5 (Bone morphogenetic protein 5), BRD1 (Bromodomain containing 1), BTC (Betacellulin), BTNL8 (Butyrophilin-like protein 8), CALB1 (Calbindin 1), CALM1 (Calmodulin 1), CAMK1 (Calcium/calmodulin-dependent protein kinase type I), CAMK4 (Calcium/calmodulin-dependent protein kinase type IV), CAMKIIB (Calcium/calmodulin-dependent protein kinase type IIB), CAMKIIG (Calcium/calmodulin-dependent protein kinase type IIG), CASP11 (Caspase-10), CASP8 (Caspase 8 apoptosis-related cysteine peptidase), CBLN1 (cerebellin 1 precursor), CCL2 (Chemokine (C-C motif) ligand 2), CCL22 (Chemokine (C-C motif) ligand 22), CCL3 (Chemokine (C-C motif) ligand 3), CCL8 (Chemokine (C-C motif) ligand 8), CCNG1 (Cyclin-G1), CCNT2 (Cyclin T2), CCR4 (C-C chemokine receptor type 4 (CD194)), CD58 (CD58), CD59 (Protectin), CD5L (CD5 antigen-like), CD93 (CD93), CDKN2AIP (CDKN2A interacting protein), CDKN2B (Cyclin-dependent kinase inhibitor 2B), CDX1 (Homeobox protein CDX-1), CEA (Carcinoembryonic antigen), CEBPA (CCAAT/enhancer-binding protein alpha), CEBPB (CCAAT/enhancer binding protein C/EBP beta), CEBPB (CCAAT/enhancer-binding protein beta), CEBPD (CCAAT/enhancer-binding protein delta), CEBPG (CCAAT/enhancer-binding protein gamma), CENPB (Centromere protein B), CGA (Glycoprotein hormone alpha chain), CGGBP1 (CGG triplet repeat-binding protein 1), CHGA (Chromogranin A), CHGB (Secretoneurin), CHN2 (Beta-chimaerin), CHRD (Chordin), CHRM1 (Cholinergic receptor muscarinic 1), CITED2 (Cbp/p300-interacting transactivator 2), CLEC4E (C-type lectin domain family 4 member E), CMTM2 (CKLF-like MARVEL transmembrane domain-containing protein 2), CNTN1 (Contactin 1), CNTNAP1 (Contactin-associated protein-like 1), CR1 (Erythrocyte complement receptor 1), CREM (cAMP-responsive element modulator), CRH (Corticotropin-releasing hormone), CRHR1 (Corticotropin releasing hormone receptor 1), CRKRS (Cell division cycle 2-related protein kinase 7), CSDA (DNA-binding protein A), CSF3 (Granulocyte colony stimulating factor 3), CSF3R (Granulocyte colony-stimulating factor 3 receptor), CSP (Chemosensory protein), CSPG4 (Chondroitin sulfate proteoglycan 4), CTCF (CCCTC-binding factor zinc finger protein), CTGF (Connective tissue growth factor), CXCL12 (Chemokine C-X-C motif ligand 12), DAD1 (Defender against cell death 1), DAXX (Death associated protein 6), DBN1 (Drebrin 1), DBP (D site of albumin promoter-albumin D-box binding protein), DDR1 (Discoidin domain receptor family member 1), DDX14 (DEAD/DEAN box helicase), DEFA3 (Defensin alpha 3 neutrophil-specific), DVL3 (Dishevelled dsh homolog 3), EDN1 (Endothelin 1), EDNRA (Endothelin receptor type A), EGF (Epidermal growth factor), EGFR (Epidermal growth factor receptor), EGR1 (Early growth response protein 1), EGR2 (Early growth response protein 2), EGR3 (Early growth response protein 3), EIF2AK2 (Eukaryotic translation initiation factor 2-alpha kinase 2), ELANE (Elastase neutrophil expressed), ELK1 (ELK1 member of ETS oncogene family), ELK3 (ELK3 ETS-domain protein (SRF accessory protein 2)), EML2 (Echinoderm microtubule associated protein like 2), EPHA4 (EPH receptor A4), ERBB2 (V-erb-b2 erythroblastic leukemia viral oncogene homolog 2), ERBB3 (Receptor tyrosine-protein kinase erbB-3), ESR2 (Estrogen receptor 2), ESR2 (Estrogen receptor 2), ETS1 (V-ets erythroblastosis virus E26 oncogene homolog 1), ETV6 (Ets variant 6), FASLG (Fas ligand TNF superfamily member 6), FCAR (Fc fragment of IgA receptor), FCER1G (Fc fragment of IgE high affinity I receptor for gamma polypeptide), FCGR2A (Fc fragment of IgG low affinity IIa receptor—CD32), FCGR3B (Fc fragment of IgG low affinity IIIb receptor—CD16b), FCGRT (Fc fragment of IgG receptor transporter alpha), FGA (Basic fibrinogen), FGF1 (Acidic fibroblast growth factor 1), FGF14 (Fibroblast growth factor 14), FGF16 (fibroblast growth factor 16), FGF18 (Fibroblast growth factor 18), FGF2 (Basic fibroblast growth factor 2), FIBP (Acidic fibroblast growth factor intracellular binding protein), FIGF (C-fos induced growth factor), FMR1 (Fragile X mental retardation 1), FOSB (FBJ murine osteosarcoma viral oncogene homolog B), FOXO1 (Forkhead box O1), FSHB (Follicle stimulating hormone beta polypeptide), FTH1 (Ferritin heavy polypeptide 1), FTL (Ferritin light polypeptide), G1P3 (Interferon alpha-inducible protein 6), G6S(N-acetylglucosamine-6-sulfatase), GABRA2 (Gamma-aminobutyric acid A receptor alpha 2), GABRA3 (Gamma-aminobutyric acid A receptor alpha 3), GABRA4 (Gamma-aminobutyric acid A receptor alpha 4), GABRB1 (Gamma-aminobutyric acid A receptor beta 1), GABRG1 (Gamma-aminobutyric acid A receptor gamma 1), GADD45A (Growth arrest and DNA-damage-inducible alpha), GCLC (Glutamate-cysteine ligase catalytic subunit), GDF15 (Growth differentiation factor 15), GDF9 (Growth differentiation factor 9), GFRA1 (GDNF family receptor alpha 1), GIT1 (G protein-coupled receptor kinase interactor 1), GNA13 (Guanine nucleotide-binding protein/G protein alpha 13), GNAQ (Guanine nucleotide binding protein/G protein q polypeptide), GPR12 (G protein-coupled receptor 12), GPR18 (G protein-coupled receptor 18), GPR22 (G protein-coupled receptor 22), GPR26 (G protein-coupled receptor 26), GPR27 (G protein-coupled receptor 27), GPR77 (G protein-coupled receptor 77), GPR85 (G protein-coupled receptor 85), GRB2 (Growth factor receptor-bound protein 2), GRLF1 (Glucocorticoid receptor DNA binding factor 1), GST (Glutathione S-transferase), GTF2B (General transcription factor IIB), GZMB (Granzyme B), HAND1 (Heart and neural crest derivatives expressed 1), HAVCR1 (Hepatitis A virus cellular receptor 1), HES1 (Hairy and enhancer of split 1), HES5 (Hairy and enhancer of split 5), HLA-DQA1 (Major histocompatibility complex class II DQ alpha), HOXA2 (Homeobox A2), HOXA4 (Homeobox A4), HP (Haptoglobin), HPGDS (Prostaglandin-D synthase), HSPA8 (Heat shock 70 kDa protein 8), HTR1A (5-hydroxytryptamine receptor 1A), HTR2A (5-hydroxytryptamine receptor 2A), HTR3A (5-hydroxytryptamine receptor 3A), ICAM1 (Intercellular adhesion molecule 1 (CD54)), IFIT2 (Interferon-induced protein with tetratricopeptide repeats 2), IFNAR2 (Interferon alpha/beta/omega receptor 2), IGF1 (Insulin-like growth factor 1), IGF2 (Insulin-like growth factor 2), IGFBP2 (Insulin-like growth factor binding protein 2, 36 kDa), IGFBP7 (Insulin-like growth factor binding protein 7), IL10 (Interleukin 10), IL10RA (Interleukin 10 receptor alpha), IL11 (Interleukin 11), IL11RA (Interleukin 11 receptor alpha), IL11RB (Interleukin 11 receptor beta), IL13 (Interleukin 13), IL15 (Interleukin 15), IL17A (Interleukin 17A), IL17RB (interleukin 17 receptor B), IL18 (Interleukin 18), IL18RAP (Interleukin 18 receptor accessory protein), IL1R2 (Interleukin 1 receptor type II), IL1RN (Interleukin 1 receptor antagonist), IL2RA (Interleukin 2 receptor alpha), IL4R (Interleukin 4 receptor), IL6 (Interleukin 6), IL6R (Interleukin 6 receptor), IL7 (Interleukin 7), IL8 (Interleukin 8), IL8RA (Interleukin 8 receptor alpha), IL8RB (Interleukin 8 receptor beta), ILK (Integrin-linked kinase), INPP4A (Inositol polyphosphate-4-phosphatase type I, 107 kDa), INPP4B (Inositol polyphosphate-4-phosphatase type 1 beta), INS (Insulin), IRF2 (Interferon regulatory factor 2), IRF3 (Interferon regulatory factor 3), IRF9 (Interferon regulatory factor 9), IRS1 (Insulin receptor substrate 1), ITGA4 (integrin alpha 4), ITGA6 (Integrin alpha-6), ITGAE (Integrin alpha E), ITGAV (Integrin alpha-V), JAG1 (Jagged 1), JAK1 (Janus kinase 1), JDP2 (Jun dimerization protein 2), JUN (Jun oncogene), JUNB (Jun B proto-oncogene), KCNJ15 (Potassium inwardly-rectifying channel subfamily J member 15), KIF5B (Kinesin family member 5B), KLRC4 (Killer cell lectin-like receptor subfamily C member 4), KRT8 (Keratin 8), LAMP2 (Lysosomal-associated membrane protein 2), LEP (Leptin), LHB (Luteinizing hormone beta polypeptide), LRRN3 (Leucine rich repeat neuronal 3), MAL (Mal T-cell differentiation protein), MAN1A1 (Mannosidase alpha class 1A member 1), MAOB (Monoamine oxidase B), MAP3K1 (Mitogen-activated protein kinase kinase kinase 1), MAPK1 (Mitogen-activated protein kinase 1), MAPK3 (Mitogen-activated protein kinase 3), MAPRE2 (Microtubule-associated protein RP/EB family member 2), MARCKS (Myristoylated alanine-rich protein kinase C substrate), MAS1 (MAS1 oncogene), MASL1 (MAS1 oncogene-like), MBP (Myelin basic protein), MCL1 (Myeloid cell leukemia sequence 1), MDMX (MDM2-like p53-binding protein), MECP2 (Methyl CpG binding protein 2), MFGE8 (Milk fat globule-EGF factor 8 protein), MIF (Macrophage migration inhibitory factor), MMP2 (Matrix metallopeptidase 2), MOBP (Myelin-associated oligodendrocyte basic protein), MUC16 (Cancer antigen 125), MX2 (Myxovirus (influenza virus) resistance 2), MYBBP1A (MYB binding protein 1a), NBN (Nibrin), NCAM1 (Neural cell adhesion molecule 1), NCF4 (Neutrophil cytosolic factor 4 40 kDa), NCOA1 (Nuclear receptor coactivator 1), NCOA2 (Nuclear receptor coactivator 2), NEDD9 (Neural precursor cell expressed developmentally down-regulated 9), NEUR (Neuraminidase), NFATC1 (Nuclear factor of activated T-cells cytoplasmic calcineurin-dependent 1), NFE2L2 (Nuclear factor erythroid-derived 2-like 2), NFIC (Nuclear factor I/C), NFKBIA (Nuclear factor of kappa light polypeptide gene enhancer in B-cells inhibitor alpha), NGFR (Nerve growth factor receptor), NIACR2 (niacin receptor 2), NLGN3 (Neuroligin 3), NPFFR2 (neuropeptide FF receptor 2), NPY (Neuropeptide Y), NR3C2 (Nuclear receptor subfamily 3 group C member 2), NRAS (Neuroblastoma RAS viral (v-ras) oncogene homolog), NRCAM (Neuronal cell adhesion molecule), NRG1 (Neuregulin 1), NRTN (Neurturin), NRXN1 (Neurexin 1), NSMAF (Neutral sphingomyelinase activation associated factor), NTF3 (Neurotrophin 3), NTF5 (Neurotrophin 4/5), ODC1 (Ornithine decarboxylase 1), OR10A1 (Olfactory receptor 10A1), OR1A1 (Olfactory receptor family 1 subfamily A member 1), OR1N1 (Olfactory receptor family 1 subfamily N member 1), OR3A2 (Olfactory receptor family 3 subfamily A member 2), OR7A17 (Olfactory receptor family 7 subfamily A member 17), ORM1 (Orosomucoid 1), OXTR (Oxytocin receptor), P2RY13 (Purinergic receptor P2Y G-protein coupled 13), P2Y12 (Purinergic receptor P2Y G-protein coupled 12), P70S6K (P70S6 kinase), PAK1 (P21/Cdc42/Rac1-activated kinase 1), PAR1 (Prader-Willi/Angelman region-1), PBEF1 (Pre-B-cell colony enhancing factor 1), PCAF (P300/CBP-associated factor), PDE4A (cAMP-specific 3′,5′-cyclic phosphodiesterase 4A), PDE4B (Phosphodiesterase 4B cAMP-specific), PDE4B (Phosphodiesterase 4B cAMP-specific), PDE4D (Phosphodiesterase 4D cAMP-specific), PDGFA (Platelet-derived growth factor alpha polypeptide), PDGFB (Platelet-derived growth factor beta polypeptide), PDGFC (Platelet derived growth factor C), PDGFRB (Beta-type platelet-derived growth factor receptor), PDPN (Podoplanin), PENK (Enkephalin), PER1 (Period homolog 1), PLA2 (Phospholipase A2), PLAU (Plasminogen activator urokinase), PLXNC1 (Plexin C1), PMVK (Phosphomevalonate kinase), PNOC (Prepronociceptin), POLH (Polymerase (DNA directed) eta), POMC (Proopiomelanocortin (adrenocorticotropin/beta-lipotropin/alpha-melanocyte stimulating hormone/beta-melanocyte stimulating hormone/beta-endorphin)), POU2AF1 (POU domain class 2 associating factor 1), PRKAA1 (5′-AMP-activated protein kinase catalytic subunit alpha-1), PRL (Prolactin), PSCDBP (Cytohesin 1 interacting protein), PSPN (Persephin), PTAFR (Platelet-activating factor receptor), PTGS2 (Prostaglandin-endoperoxide synthase 2), PTN (Pleiotrophin), PTPN11 (Protein tyrosine phosphatase non-receptor type 11), PYY (Peptide YY), RAB11B (RAB11B member RAS oncogene family), RAB6A (RAB6A member RAS oncogene family), RAD17 (RAD17 homolog), RAF1 (RAF proto-oncogene serine/threonine-protein kinase), RANBP2 (RAN binding protein 2), RAP1A (RAP1A member of RAS oncogene family), RB1 (Retinoblastoma 1), RBL2 (Retinoblastoma-like 2 (p130)), RCVRN (Recoverin), REM2 (RAS/RAD/GEM-like GTP binding 2), RFRP (RFamide-related peptide), RPS6KA3 (Ribosomal protein S6 kinase 90 kDa polypeptide 3), RTN4 (Reticulon 4), RUNX1 (Runt-related transcription factor 1), S100A4 (S100 calcium binding protein A4), S1PR1 (Sphingosine-1-phosphate receptor 1), SCG2 (Secretogranin II), SCYE1 (Small inducible cytokine subfamily E member 1), SELENBP1 (Selenium binding protein 1), SGK (Serum/glucocorticoid regulated kinase), SKD1 (Suppressor of K+ transport growth defect 1), SLC14A1 (Solute carrier family 14 (urea transporter) member 1 (Kidd blood group)), SLC25A37 (Solute carrier family 25 member 37), SMAD2 (SMAD family member 2), SMAD5 (SMAD family member 5), SNAP23 (Synaptosomal-associated protein 23 kDa), SNCB (Synuclein beta), SNF1LK (SNF1-like kinase), SORT1 (Sortilin 1), SSB (Sjogren syndrome antigen B), STAT1 (Signal transducer and activator of transcription 1, 91 kDa), STAT5A (Signal transducer and activator of transcription 5A), STAT5B (Signal transducer and activator of transcription 5B), STX16 (Syntaxin 16), TAC1 (Tachykinin precursor 1), TBX1 (T-box 1), TEF (Thyrotrophic embryonic factor), TF (Transferrin), TGFA (Transforming growth factor alpha), TGFB1 (Transforming growth factor beta 1), TGFB2 (Transforming growth factor beta 2), TGFB3 (Transforming growth factor beta 3), TGFBR1 (Transforming growth factor beta receptor I), TGM2 (Transglutaminase 2), THPO (Thrombopoietin), TIMP1 (TIMP metallopeptidase inhibitor 1), TIMP3 (TIMP metallopeptidase inhibitor 3), TMEM129 (Transmembrane protein 129), TNFRC6 (TNFR/NGFR cysteine-rich region), TNFRSF10A (Tumor necrosis factor receptor superfamily member 10a), TNFRSF10C (Tumor necrosis factor receptor superfamily member 10c decoy without an intracellular domain), TNFRSF1A (Tumor necrosis factor receptor superfamily member 1A), TOB2 (Transducer of ERBB2 2), TOP1 (Topoisomerase (DNA) I), TOPOII (Topoisomerase 2), TRAK2 (Trafficking protein kinesin binding 2), TRH (Thyrotropin-releasing hormone), TSH (Thyroid-stimulating hormone alpha), TUBA1A (Tubulin alpha 1a), TXK (TXK tyrosine kinase), TYK2 (Tyrosine kinase 2), UCP1 (Uncoupling protein 1), UCP2 (Uncoupling protein 2), ULIP (Unc-33-like phosphoprotein), UTRN (Utrophin), VEGF (Vascular endothelial growth factor), VGF (VGF nerve growth factor inducible), VIP (Vasoactive intestinal peptide), VNN1 (Vanin 1), VTN (Vitronectin), WNT2 (Wingless-type MMTV integration site family member 2), XRCC6 (X-ray repair cross-complementing 6), ZEB2 (Zinc finger E-box binding homeobox 2), and ZNF461 (Zinc finger protein 461).

Exemplary prion disorder-related proteins, include PrP^{C} and isoforms thereof, PrP^{Sc} and isoforms thereof, HECTD2 (e3-ubipuitin ligase protein), STI1 (stress inducible protein 1), DPL (residue Doppel protein, encoded by Prnd), APOA1 (Apolipoprotein A1), BCL-2 (B-cell lymphoma 2), HSP60 (Heat shock 60 kDa protein), BAX-inhibiting peptide (Bcl-2-associated X protein inhibitor), NRF2 (nuclear respiratory factor 2), NCAMs (neural cell-adhesion molecules), heparin, laminin and laminin receptor and any combination thereof.

In certain embodiments, an animal created by a method of the invention may be used to study the effects of mutations on the animal and development and/or progression of a prion disorder using measures commonly used in the study of prion disorders.

### M. Immunodeficiency

In one embodiment, a method of the invention may be used to create an animal or cell in which at least one chromosomal sequence associated with immunodeficiency has been edited. Suitable chromosomal edits may include, but are not limited to, the type of edits detailed in section I(f) above.

In each of the above embodiments, one or more chromosomal sequences associated with immunodeficiency may be edited. An immunodeficiency protein or control sequence is a protein or control sequence for which an alteration in activity is linked to an immunodeficiency, which may be the primary or a secondary symptom of an animal disease or condition, preferably a mammalian, e.g., a human, disease or condition. An immunodeficiency sequence may typically be selected based on an experimental association of the immunodeficiency sequence to an immunodeficiency disease or condition, especially a mammalian, e.g., a human, disease or condition. For example, the expression of an immunodeficiency protein in a particular tissue may be elevated or depressed in a population having an immunodeficiency disease or condition relative to a population lacking the disease or condition. Differences in protein levels may be assessed using proteomic or genomic analysis techniques known in the art.

Non-limiting examples of human immunodeficiency genes include A2M [alpha-2-macroglobulin]; AANAT [arylalkylamine N-acetyltransferase]; ABCA1 [ATP-binding cassette, sub-family A (ABC1), member 1]; ABCA2 [ATP-binding cassette, sub-family A (ABC1), member 2]; ABCA3 [ATP-binding cassette, sub-family A (ABC1), member 3]; ABCA4 [ATP-binding cassette, sub-family A (ABC1), member 4]; ABCB1 [ATP-binding cassette, sub-family B (MDR/TAP), member 1]; ABCC1 [ATP-binding cassette, sub-family C (CFTR/MRP), member 1]; ABCC2 [ATP-binding cassette, sub-family C (CFTR/MRP), member 2]; ABCC3 [ATP-binding cassette, sub-family C (CFTR/MRP), member 3]; ABCC4 [ATP-binding cassette, sub-family C (CFTR/MRP), member 4]; ABCC8 [ATP-binding cassette, sub-family C (CFTR/MRP), member 8]; ABCD2 [ATP-binding cassette, sub-family D (ALD), member 2]; ABCD3 [ATP-binding cassette, sub-family D (ALD), member 3]; ABCG1 [ATP-binding cassette, sub-family G (WHITE), member 1]; ABCC2 [ATP-binding cassette, sub-family G (WHITE), member 2]; ABCG5 [ATP-binding cassette, sub-family G (WHITE), member 5]; ABCC8 [ATP-binding cassette, sub-family G (WHITE), member 8]; ABHD2 [abhydrolase domain containing 2]; ABL1 [c-abl oncogene 1, receptor tyrosine kinase]; ABO [ABO blood group (transferase A, alpha 1-3-N-acetylgalactosaminyltransferase; transferase B, alpha 1-3-galactosyltransferase)]; ABP1 [amiloride binding protein 1 (amine oxidase (copper-containing))]; ACAA1 [acetyl-Coenzyme A acyltransferase 1]; ACACA [acetyl-Coenzyme A carboxylase alpha]; ACAN [aggrecan]; ACAT1 [acetyl-Coenzyme A acetyltransferase 1]; ACAT2 [acetyl-Coenzyme A acetyltransferase 2]; ACCN5 [amiloride-sensitive cation channel 5, intestinal]; ACE [angiotensin I converting enzyme (peptidyl-dipeptidase A) 1]; ACE2 [angiotensin I converting enzyme (peptidyl-dipeptidase A) 2]; ACHE [acetylcholinesterase (Yt blood group)]; ACLY [ATP citrate lyase]; ACOT9 [acyl-CoA thioesterase 9]; ACOX1 [acyl-Coenzyme A oxidase 1, palmitoyl]; ACP1 [acid phosphatase 1, soluble]; ACP2 [acid phosphatase 2, lysosomal]; ACP5 [acid phosphatase 5, tartrate resistant]; ACPP [acid phosphatase, prostate]; ACSL3 [acyl-CoA synthetase long-chain family member 3]; ACSM3 [acyl-CoA synthetase medium-chain family member 3]; ACTA1 [actin, alpha 1, skeletal muscle]; ACTA2 [actin, alpha 2, smooth muscle, aorta]; ACTB [actin, beta]; ACTC1 [actin, alpha, cardiac muscle 1]; ACTG1 [actin, gamma 1]; ACTN1 [actinin, alpha 1]; ACTN2 [actinin, alpha 2]; ACTN4 [actinin, alpha 4]; ACTR2 [ARP2 actin-related protein 2 homolog (yeast)]; ACVR1 [activin A receptor, type I]; ACVR1B [activin A receptor, type IB]; ACVRL1 [activin A receptor type II-like 1]; ACY1 [aminoacylase 1]; ADA [adenosine deaminase]; ADAM10 [ADAM metallopeptidase domain 10]; ADAM12 [ADAM metallopeptidase domain 12]; ADAM17 [ADAM metallopeptidase domain 17]; ADAM23 [ADAM metallopeptidase domain 23]; ADAM33 [ADAM metallopeptidase domain 33]; ADAM8 [ADAM metallopeptidase domain 8]; ADAM9 [ADAM metallopeptidase domain 9 (meltrin gamma)]; ADAMTS1 [ADAM metallopeptidase with thrombospondin type 1 motif, 1]; ADAMTS12 [ADAM metallopeptidase with thrombospondin type 1 motif, 12]; ADAMTS13 [ADAM metallopeptidase with thrombospondin type 1 motif, 13]; ADAMTS15 [ADAM metallopeptidase with thrombospondin type 1 motif, 15]; ADAMTSL1 [ADAMTS-like 1]; ADAMTSL4 [ADAMTS-like 4]; ADAR [adenosine deaminase, RNA-specific]; ADCY1 [adenylate cyclase 1 (brain)]; ADCY10 [adenylate cyclase 10 (soluble)]; ADCY3 [adenylate cyclase 3]; ADCY9 [adenylate cyclase 9]; ADCYAP1 [adenylate cyclase activating polypeptide 1 (pituitary)]; ADCYAP1R1 [adenylate cyclase activating polypeptide 1 (pituitary) receptor type I]; ADD1 [adducin 1 (alpha)]; ADH5 [alcohol dehydrogenase 5 (class III), chi polypeptide]; ADIPOQ [adiponectin, C1Q and collagen domain containing]; ADIPOR1 [adiponectin receptor 1]; ADK [adenosine kinase]; ADM [adrenomedullin]; ADORA1 [adenosine A1 receptor]; ADORA2A [adenosine A2a receptor]; ADORA2B [adenosine A2b receptor]; ADORA3 [adenosine A3 receptor]; ADRA1B [adrenergic, alpha-1B-, receptor]; ADRA2A [adrenergic, alpha-2A-, receptor]; ADRA2B [adrenergic, alpha-2B-, receptor]; ADRB1 [adrenergic, beta-1-, receptor]; ADRB2 [adrenergic, beta-2-, receptor, surface]; ADSL [adenylosuccinate lyase]; ADSS [adenylosuccinate synthase]; AEBP1 [AE binding protein 1]; AFP [alpha-fetoprotein]; AGER [advanced glycosylation end product-specific receptor]; AGMAT [agmatine ureohydrolase (agmatinase)]; AGPS [alkylglycerone phosphate synthase]; AGRN [agrin]; AGRP [agouti related protein homolog (mouse)]; AGT [angiotensinogen (serpin peptidase inhibitor, clade A, member 8)]; AGTR1 [angiotensin II receptor, type 1]; AGTR2 [angiotensin II receptor, type 2]; AHOY [adenosylhomocysteinase]; AH11 [Abelson helper integration site 1]; AHR [aryl hydrocarbon receptor]; AHSP [alpha hemoglobin stabilizing protein]; AICDA [activation-induced cytidine deaminase]; AIDA [axin interactor, dorsalization associated]; AIMP1 [aminoacyl tRNA synthetase complex-interacting multifunctional protein 1]; AIRE [autoimmune regulator]; AK1 [adenylate kinase 1]; AK2 [adenylate kinase 2]; AKR1A1 [aldo-keto reductase family 1, member A1 (aldehyde reductase)]; AKR1B1 [aldo-keto reductase family 1, member B1 (aldose reductase)]; AKR1C3 [aldo-keto reductase family 1, member C3 (3-alpha hydroxysteroid dehydrogenase, type II)]; AKT1 [v-akt murine thymoma viral oncogene homolog 1]; AKT2 [v-akt murine thymoma viral oncogene homolog 2]; AKT3 [v-akt murine thymoma viral oncogene homolog 3 (protein kinase B, gamma)]; ALB [albumin]; ALCAM [activated leukocyte cell adhesion molecule]; ALDH1A1 [aldehyde dehydrogenase 1 family, member A1]; ALDH2 [aldehyde dehydrogenase 2 family (mitochondrial)]; ALDH3A1 [aldehyde dehydrogenase 3 family, memberA1]; ALDH7A1 [aldehyde dehydrogenase 7 family, member A1]; ALDH9A1 [aldehyde dehydrogenase 9 family, member A1]; ALG1 [asparagine-linked glycosylation 1, beta-1,4-mannosyltransferase homolog (S. cerevisiae)]; ALG12 [asparagine-linked glycosylation 12, alpha-1,6-mannosyltransferase homolog (S. cerevisiae)]; ALK [anaplastic lymphoma receptor tyrosine kinase]; ALOX12 [arachidonate 12-lipoxygenase]; ALOX15 [arachidonate 15-lipoxygenase]; ALOX15B [arachidonate 15-lipoxygenase, type B]; ALOX5 [arachidonate 5-lipoxygenase]; ALOX5AP [arachidonate 5-lipoxygenase-activating protein]; ALPI [alkaline phosphatase, intestinal]; ALPL [alkaline phosphatase, liver/bone/kidney]; ALPP [alkaline phosphatase, placental (Regan isozyme)]; AMACR [alpha-methylacyl-CoA racemase]; AMBP [alpha-1-microglobulin/bikunin precursor]; AMPD3 [adenosine monophosphate deaminase 3]; ANG [angiogenin, ribonuclease, RNase A family, 5]; ANGPT1 [angiopoietin 1]; ANGPT2 [angiopoietin 2]; ANK1 [ankyrin 1, erythrocytic]; ANKH [ankylosis, progressive homolog (mouse)]; ANKRD1 [ankyrin repeat domain 1 (cardiac muscle)]; ANPEP [alanyl (membrane) aminopeptidase]; ANTXR2 [anthrax toxin receptor 2]; ANXA1 [annexin A1]; ANXA2 [annexin A2]; ANXA5 [annexin A5]; ANXA6 [annexin A6]; AOAH [acyloxyacyl hydrolase (neutrophil)]; AOC2 [amine oxidase, copper containing 2 (retina-specific)]; AP2B1 [adaptor-related protein complex 2, beta 1 subunit]; AP3B1 [adaptor-related protein complex 3, beta 1 subunit]; APC [adenomatous polyposis coli]; APCS [amyloid P component, serum]; APEX1 [APEX nuclease (multifunctional DNA repair enzyme) 1]; APLNR [apelin receptor]; APOA1 [apolipoprotein A-I]; APOA2 [apolipoprotein A-II]; APOA4 [apolipoprotein A-IV]; APOB [apolipoprotein B (including Ag(x) antigen)]; APOBEC1 [apolipoprotein B mRNA editing enzyme, catalytic polypeptide 1]; APOBEC3G [apolipoprotein B mRNA editing enzyme, catalytic polypeptide-like 3G]; APOC3 [apolipoprotein C-III]; APOD [apolipoprotein D]; APOE [apolipoprotein E]; APOH [apolipoprotein H (beta-2-glycoprotein I)]; APP [amyloid beta (A4) precursor protein]; APRT [adenine phosphoribosyltransferase]; APTX [aprataxin]; AQP1 [aquaporin 1 (Colton blood group)]; AQP2 [aquaporin 2 (collecting duct)]; AQP3 [aquaporin 3 (Gill blood group)]; AQP4 [aquaporin 4]; AQP5 [aquaporin 5]; AQP7 [aquaporin 7]; AQP8 [aquaporin 8]; AR [androgen receptor]; AREG [amphiregulin]; ARF6 [ADP-ribosylation factor 6]; ARG1 [arginase, liver]; ARG2 [arginase, type II]; ARHGAP6 [Rho GTPase activating protein 6]; ARHGEF2 [Rho/Rac guanine nucleotide exchange factor (GEF) 2]; ARHGEF6 [Rac/Cdc42 guanine nucleotide exchange factor (GEF) 6]; ARL13B [ADP-ribosylation factor-like 13B]; ARNT [aryl hydrocarbon receptor nuclear translocator]; ARNTL [aryl hydrocarbon receptor nuclear translocator-like]; ARRB1 [arrestin, beta 1]; ARRB2 [arrestin, beta 2]; ARSA [arylsulfatase A]; ARSB [arylsulfatase B]; ARSH [arylsulfatase family, member H]; ART1 [ADP-ribosyltransferase 1]; ASAH1 [N-acylsphingosine amidohydrolase (acid ceramidase) 1]; ASAP1 [ArfGAP with SH3 domain, ankyrin repeat and PH domain 1]; ASGR2 [asialoglycoprotein receptor 2]; ASL [argininosuccinate lyase]; ASNS [asparagine synthetase]; ASPA [aspartoacylase (Canavan disease)]; ASPG [asparaginase homolog (S. cerevisiae)]; ASPH [aspartate beta-hydroxylase]; ASRGL1 [asparaginase like 1]; ASS1 [argininosuccinate synthase 1]; ATF1 [activating transcription factor 1]; ATF2 [activating transcription factor 2]; ATF3 [activating transcription factor 3]; ATF4 [activating transcription factor 4 (tax-responsive enhancer element B67)]; ATG16L1 [ATG16 autophagy related 16-like 1 (S. cerevisiae)]; ATM [ataxia telangiectasia mutated]; ATMIN [ATM interactor]; ATN1 [atrophin 1]; ATOH1 [atonal homolog 1 (Drosophila)]; ATP2A2 [ATPase, Ca++ transporting, cardiac muscle, slow twitch 2]; ATP2A3 [ATPase, Ca++ transporting, ubiquitous]; ATP2C1 [ATPase, Ca++ transporting, type 2C, member 1]; ATP5E [ATP synthase, H+ transporting, mitochondrial F1 complex, epsilon subunit]; ATP7B [ATPase, Cu++ transporting, beta polypeptide]; ATP8B1 [ATPase, class I, type 8B, member 1]; ATPAF2 [ATP synthase mitochondrial F1 complex assembly factor 2]; ATR [ataxia telangiectasia and Rad3 related]; ATRIP [ATR interacting protein]; ATRN [attractin]; AURKA [aurora kinase A]; AURKB [aurora kinase B]; AURKC [aurora kinase C]; AVP [arginine vasopressin]; AVPR2 [arginine vasopressin receptor 2]; AXL [AXL receptor tyrosine kinase]; AZGP1 [alpha-2-glycoprotein 1, zinc-binding]; B2M [beta-2-microglobulin]; B3GALTL [beta 1,3-galactosyltransferase-like]; B3GAT1 [beta-1,3-glucuronyltransferase 1 (glucuronosyltransferase P)]; B4GALNT1 [beta-1,4-N-acetyl-galactosaminyl transferase 1]; B4GALT1 [UDP-Gal:betaGlcNAc beta 1,4-galactosyltransferase, polypeptide 1]; BACE1 [beta-site APP-cleaving enzyme 1]; BACE2 [beta-site APP-cleaving enzyme 2]; BACH1 [BTB and CNC homology 1, basic leucine zipper transcription factor 1]; BAD [BCL2-associated agonist of cell death]; BAIAP2 [BAI1-associated protein 2]; BAK1 [BCL2-antagonist/killer 1]; BARX2 [BARX homeobox 2]; BAT1 [HLA-B associated transcript 1]; BAT2 [HLA-B associated transcript 2]; BAX [BCL2-associated X protein]; BBC3 [BCL2 binding component 3]; BCAR1 [breast cancer anti-estrogen resistance 1]; BCAT1 [branched chain aminotransferase 1, cytosolic]; BCAT2 [branched chain aminotransferase 2, mitochondrial]; BCHE [butyrylcholinesterase]; BCL10 [B-cell CLL/lymphoma 10]; BCL11B [B-cell CLL/lymphoma 11B (zinc finger protein)]; BCL2 [B-cell CLL/lymphoma 2]; BCL2A1 [BCL2-related protein A1]; BCL2L1 [BCL2-like 1]; BCL2L11 [BCL2-like 11 (apoptosis facilitator)]; BCL3 [B-cell CLL/lymphoma 3]; BCL6 [B-cell CLL/lymphoma 6]; BCR [breakpoint cluster region]; BDKRB1 [bradykinin receptor B1]; BDKRB2 [bradykinin receptor B2]; BDNF [brain-derived neurotrophic factor]; BECN1 [beclin 1, autophagy related]; BEST1 [bestrophin 1]; BFAR [bifunctional apoptosis regulator]; BGLAP [bone gamma-carboxyglutamate (gla) protein]; BHMT [betaine-homocysteine methyltransferase]; BID [BH3 interacting domain death agonist]; BIK [BCL2-interacting killer (apoptosis-inducing)]; BIRC2 [baculoviral IAP repeat-containing 2]; BIRC3 [baculoviral IAP repeat-containing 3]; BIRC5 [baculoviral IAP repeat-containing 5]; BLK [B lymphoid tyrosine kinase]; BLM [Bloom syndrome, RecQ helicase-like]; BLNK [B-cell linker]; BLVRB [biliverdin reductase B (flavin reductase (NADPH))]; BMI1 [BMI1 polycomb ring finger oncogene]; BMP1 [bone morphogenetic protein 1]; BMP2 [bone morphogenetic protein 2]; BMP4 [bone morphogenetic protein 4]; BMP6 [bone morphogenetic protein 6]; BMP7 [bone morphogenetic protein 7]; BMPR1A [bone morphogenetic protein receptor, type IA]; BMPR1B [bone morphogenetic protein receptor, type IB]; BMPR2 [bone morphogenetic protein receptor, type II (serine/threonine kinase)]; BPI [bactericidal/permeability-increasing protein]; BRCA1 [breast cancer 1, early onset]; BRCA2 [breast cancer 2, early onset]; BRCC3 [BRCA1/BRCA2-containing complex, subunit 3]; BRD8 [bromodomain containing 8]; BRIP1 [BRCA1 interacting protein C-terminal helicase 1]; BSG [basigin (Ok blood group)]; BSN [bassoon (presynaptic cytomatrix protein)]; BSX [brain-specific homeobox]; BTD [biotinidase]; BTK [Bruton agammaglobulinemia tyrosine kinase]; BTLA [B and T lymphocyte associated]; BTNL2 [butyrophilin-like 2 (MHC class II associated)]; BTRC [beta-transducin repeat containing]; C10orf67 [chromosome 10 open reading frame 67]; C11orf30 [chromosome 11 open reading frame 30]; C11orf58 [chromosome 11 open reading frame 58]; C13orf23 [chromosome 13 open reading frame 23]; C13orf31 [chromosome 13 open reading frame 31]; C15orf2 [chromosome 15 open reading frame 2]; C16orf75 [chromosome 16 open reading frame 75]; C19orf10 [chromosome 19 open reading frame 10]; C1QA [complement component 1, q subcomponent, A chain]; C1QB [complement component 1, q subcomponent, B chain]; C1QC [complement component 1, q subcomponent, C chain]; C1QTNF5 [C1 q and tumor necrosis factor related protein 5]; C1R [complement component 1, r subcomponent]; C1S [complement component 1, s subcomponent]; C2 [complement component 2]; C20orf29 [chromosome 20 open reading frame 29]; C21orf33 [chromosome 21 open reading frame 33]; C3 [complement component 3]; C3AR1 [complement component 3a receptor 1]; C3orf27 [chromosome 3 open reading frame 27]; C4A [complement component 4A (Rodgers blood group)]; C4B [complement component 4B (Chido blood group)]; C4BPA [complement component 4 binding protein, alpha]; C4BPB [complement component 4 binding protein, beta]; C5 [complement component 5]; C5AR1 [complement component 5a receptor 1]; C5orf56 [chromosome 5 open reading frame 56]; C5orf62 [chromosome 5 open reading frame 62]; C6 [complement component 6]; C6orf142 [chromosome 6 open reading frame 142]; C6orf25 [chromosome 6 open reading frame 25]; C7 [complement component 7]; C7orf72 [chromosome 7 open reading frame 72]; C8A [complement component 8, alpha polypeptide]; C8B [complement component 8, beta polypeptide]; C8G [complement component 8, gamma polypeptide]; C8orf38 [chromosome 8 open reading frame 38]; C9 [complement component 9]; CA2 [carbonic anhydrase II]; CA6 [carbonic anhydrase VI]; CA8 [carbonic anhydrase VIII]; CA9 [carbonic anhydrase IX]; CABIN1 [calcineurin binding protein 1]; CACNA1C [calcium channel, voltage-dependent, L type, alpha 1C subunit]; CACNA1S [calcium channel, voltage-dependent, L type, alpha 1S subunit]; CAD [carbamoyl-phosphate synthetase 2, aspartate transcarbamylase, and dihydroorotase]; CALB1 [calbindin 1, 28 kDa]; CALB2 [calbindin 2]; CALCA [calcitonin-related polypeptide alpha]; CALCRL [calcitonin receptor-like]; CALD1 [caldesmon 1]; CALM1 [calmodulin 1 (phosphorylase kinase, delta)]; CALM2 [calmodulin 2 (phosphorylase kinase, delta)]; CALM3 [calmodulin 3 (phosphorylase kinase, delta)]; CALR [calreticulin]; CAMK2G [calcium/calmodulin-dependent protein kinase II gamma]; CAMP [cathelicidin antimicrobial peptide]; CANT1 [calcium activated nucleotidase 1]; CANX [calnexin]; CAPN1 [calpain 1, (mu/I) large subunit]; CARD10 [caspase recruitment domain family, member 10]; CARD16 [caspase recruitment domain family, member 16]; CARD8 [caspase recruitment domain family, member 8]; CARDS [caspase recruitment domain family, member 9]; CASP1 [caspase 1, apoptosis-related cysteine peptidase (interleukin 1, beta, convertase)]; CASP10 [caspase 10, apoptosis-related cysteine peptidase]; CASP2 [caspase 2, apoptosis-related cysteine peptidase]; CASP3 [caspase 3, apoptosis-related cysteine peptidase]; CASP5 [caspase 5, apoptosis-related cysteine peptidase]; CASP6 [caspase 6, apoptosis-related cysteine peptidase]; CASP7 [caspase 7, apoptosis-related cysteine peptidase]; CASP8 [caspase 8, apoptosis-related cysteine peptidase]; CASP8AP2 [caspase 8 associated protein 2]; CASP9 [caspase 9, apoptosis-related cysteine peptidase]; CASR [calcium-sensing receptor]; CAST [calpastatin]; CAT [catalase]; CAV1 [caveolin 1, caveolae protein, 22 kDa]; CAV2 [caveolin 2]; CBL [Cas-Br-M (murine) ecotropic retroviral transforming sequence]; CBS [cystathionine-beta-synthase]; CBX5 [chromobox homolog 5 (HP1 alpha homolog, Drosophila)]; CC2D2A [coiled-coil and C2 domain containing 2A]; CCBP2 [chemokine binding protein 2]; CCDC144A [coiled-coil domain containing 144A]; CCDC144B [coiled-coil domain containing 144B]; CCDC68 [coiled-coil domain containing 68]; CCK [cholecystokinin]; CCL1 [chemokine (C-C motif) ligand 1]; CCL11 [chemokine (C-C motif) ligand 11]; CCL13 [chemokine (C-C motif) ligand 13]; CCL14 [chemokine (C-C motif) ligand 14]; CCL17 [chemokine (C-C motif) ligand 17]; CCL18 [chemokine (C-C motif) ligand 18 (pulmonary and activation-regulated)]; CCL19 [chemokine (C-C motif) ligand 19]; CCL2 [chemokine (C-C motif) ligand 2]; CCL20 [chemokine (C-C motif) ligand 20]; CCL21 [chemokine (C-C motif) ligand 21]; CCL22 [chemokine (C-C motif) ligand 22]; CCL24 [chemokine (C-C motif) ligand 24]; CCL25 [chemokine (C-C motif) ligand 25]; CCL26 [chemokine (C-C motif) ligand 26]; CCL27 [chemokine (C-C motif) ligand 27]; CCL28 [chemokine (C-C motif) ligand 28]; CCL3 [chemokine (C-C motif) ligand 3]; CCL4 [chemokine (C-C motif) ligand 4]; CCL4L1 [chemokine (C-C motif) ligand 4-like 1]; CCL5 [chemokine (C-C motif) ligand 5]; CCL7 [chemokine (C-C motif) ligand 7]; CCL8 [chemokine (C-C motif) ligand 8]; CCNA1 [cyclin A1]; CCNA2 [cyclin A2]; CCNB1 [cyclin B1]; CCNB2 [cyclin B2]; CCNC [cyclin C]; CCND1 [cyclin D1]; CCND2 [cyclin D2]; CCND3 [cyclin D3]; CCNE1 [cyclin E1]; CCNG1 [cyclin G1]; CCNH [cyclin H]; CCNT1 [cyclin T1]; CCNT2 [cyclin T2]; CCNY [cyclin Y]; CCR1 [chemokine (C-C motif) receptor 1]; CCR2 [chemokine (C-C motif) receptor 2]; CCR3 [chemokine (C-C motif) receptor 3]; CCR4 [chemokine (C-C motif) receptor 4]; CCR5 [chemokine (C-C motif) receptor 5]; CCR6 [chemokine (C-C motif) receptor 6]; CCR7 [chemokine (C-C motif) receptor 7]; CCR8 [chemokine (C-C motif) receptor 8]; CCR9 [chemokine (C-C motif) receptor 9]; CCRL1 [chemokine (C-C motif) receptor-like 1]; CD14 [CD14 molecule]; CD151 [CD151 molecule (Raph blood group)]; CD160 [CD160 molecule]; CD163 [CD163 molecule]; CD180 [CD180 molecule]; CD19 [CD19 molecule]; CD1A [CD1a molecule]; CD1B [CD1b molecule]; CD1C [CD1c molecule]; CD1D [CD1d molecule]; CD2 [CD2 molecule]; CD200 [CD200 molecule]; CD207 [CD207 molecule, langerin]; CD209 [CD209 molecule]; CD22 [CD22 molecule]; CD226 [CD226 molecule]; CD24 [CD24 molecule]; CD244 [CD244 molecule, natural killer cell receptor 2B4]; CD247 [CD247 molecule]; CD27 [CD27 molecule]; CD274 [CD274 molecule]; CD28 [CD28 molecule]; CD2AP [CD2-associated protein]; CD300LF [CD300 molecule-like family member f]; CD34 [CD34 molecule]; CD36 [CD36 molecule (thrombospondin receptor)]; CD37 [CD37 molecule]; CD38 [CD38 molecule]; CD3E [CD3e molecule, epsilon (CD3-TCR complex)]; CD4 [CD4 molecule]; CD40 [CD40 molecule, TNF receptor superfamily member 5]; CD40LG [CD40 ligand]; CD44 [CD44 molecule (Indian blood group)]; CD46 [CD46 molecule, complement regulatory protein]; CD47 [CD47 molecule]; CD48 [CD48 molecule]; CD5 [CD5 molecule]; CD52 [CD52 molecule]; CD53 [CD53 molecule]; CD55 [CD55 molecule, decay accelerating factor for complement (Cromer blood group)]; CD58 [CD58 molecule]; CD59 [CD59 molecule, complement regulatory protein]; CD63 [CD63 molecule]; CD68 [CD68 molecule]; CD69 [CD69 molecule]; CD7 [CD7 molecule]; CD70 [CD70 molecule]; CD72 [CD72 molecule]; CD74 [CD74 molecule, major histocompatibility complex, class II invariant chain]; CD79A [CD79a molecule, immunoglobulin-associated alpha]; CD79B [CD79b molecule, immunoglobulin-associated beta]; CD80 [CD80 molecule]; CD81 [CD81 molecule]; CD82 [CD82 molecule]; CD83 [CD83 molecule]; CD86 [CD86 molecule]; CD8A [CD8a molecule]; CD9 [CD9 molecule]; CD93 [CD93 molecule]; CD97 [CD97 molecule]; CDC20 [cell division cycle 20 homolog (S. cerevisiae)]; CDC25A [cell division cycle 25 homolog A (S. pombe)]; CDC25B [cell division cycle 25 homolog B (S. pombe)]; CDC25C [cell division cycle 25 homolog C (S. pombe)]; CDC42 [cell division cycle 42 (GTP binding protein, 25 kDa)]; CDC45 [CDC45 cell division cycle 45 homolog (S. cerevisiae)]; CDC5L [CDC5 cell division cycle 5-like (S. pombe)]; CDC6 [cell division cycle 6 homolog (S. cerevisiae)]; CDC7 [cell division cycle 7 homolog (S. cerevisiae)]; CDH1 [cadherin 1, type 1, E-cadherin (epithelial)]; CDH2 [cadherin 2, type 1, N-cadherin (neuronal)]; CDH26 [cadherin 26]; CDH3 [cadherin 3, type 1, P-cadherin (placental)]; CDH5 [cadherin 5, type 2 (vascular endothelium)]; CDIPT [CDP-diacylglycerol-inositol 3-phosphatidyltransferase (phosphatidylinositol synthase)]; CDK1 [cyclin-dependent kinase 1]; CDK2 [cyclin-dependent kinase 2]; CDK4 [cyclin-dependent kinase 4]; CDK5 [cyclin-dependent kinase 5]; CDK5R1 [cyclin-dependent kinase 5, regulatory subunit 1 (p35)]; CDK7 [cyclin-dependent kinase 7]; CDK9 [cyclin-dependent kinase 9]; CDKAL1 [CDK5 regulatory subunit associated protein 1-like 1]; CDKN1A [cyclin-dependent kinase inhibitor 1A (p21, Cip1)]; CDKN1B [cyclin-dependent kinase inhibitor 1B (p27, Kip1)]; CDKN1C [cyclin-dependent kinase inhibitor 1C (p57, Kip2)]; CDKN2A [cyclin-dependent kinase inhibitor 2A (melanoma, p16, inhibits CDK4)]; CDKN2B [cyclin-dependent kinase inhibitor 2B (p15, inhibits CDK4)]; CDKN3 [cyclin-dependent kinase inhibitor 3]; CDR2 [cerebellar degeneration-related protein 2, 62 kDa]; CDT1 [chromatin licensing and DNA replication factor 1]; CDX2 [caudal type homeobox 2]; CEACAM1 [carcinoembryonic antigen-related cell adhesion molecule 1 (biliary glycoprotein)]; CEACAM3 [carcinoembryonic antigen-related cell adhesion molecule 3]; CEACAM5 [carcinoembryonic antigen-related cell adhesion molecule 5]; CEACAM6 [carcinoembryonic antigen-related cell adhesion molecule 6 (non-specific cross reacting antigen)]; CEACAM7 [carcinoembryonic antigen-related cell adhesion molecule 7]; CEBPB [CCAAT/enhancer binding protein (C/EBP), beta]; CEL [carboxyl ester lipase (bile salt-stimulated lipase)]; CENPJ [centromere protein J]; CENPV [centromere protein V]; CEP290 [centrosomal protein 290 kDa]; CERK [ceramide kinase]; CETP [cholesteryl ester transfer protein, plasma]; CFB [complement factor B]; CFD [complement factor D (adipsin)]; CFDP1 [craniofacial development protein 1]; CFH [complement factor H]; CFHR1 [complement factor H-related 1]; CFHR3 [complement factor H-related 3]; CFI [complement factor I]; CFL1 [cofilin 1 (non-muscle)]; CFL2 [cofilin 2 (muscle)]; CFLAR [CASP8 and FADD-like apoptosis regulator]; CFP [complement factor properdin]; CFTR [cystic fibrosis transmembrane conductance regulator (ATP-binding cassette sub-family C, member 7)]; CGA [glycoprotein hormones, alpha polypeptide]; CGB [chorionic gonadotropin, beta polypeptide]; CGB5 [chorionic gonadotropin, beta polypeptide 5]; CHAD [chondroadherin]; CHAF1A [chromatin assembly factor 1, subunit A (p150)]; CHAF1B [chromatin assembly factor 1, subunit B (p60)]; CHAT [choline acetyltransferase]; CHD2 [chromodomain helicase DNA binding protein 2]; CHD7 [chromodomain helicase DNA binding protein 7]; CHEK1 [CHK1 checkpoint homolog (S. pombe)]; CHEK2 [CHK2 checkpoint homolog (S. pombe)]; CHGA [chromogranin A (parathyroid secretory protein 1)]; CHGB [chromogranin B (secretogranin 1)]; CHI3L1 [chitinase 3-like 1 (cartilage glycoprotein-39)]; CHIA [chitinase, acidic]; CHIT1 [chitinase 1 (chitotriosidase)]; CHKA [choline kinase alpha]; CHML [choroideremia-like (Rab escort protein 2)]; CHRD [chordin]; CHRDL1 [chordin-like 1]; CHRM1 [cholinergic receptor, muscarinic 1]; CHRM2 [cholinergic receptor, muscarinic 2]; CHRM3 [cholinergic receptor, muscarinic 3]; CHRNA3 [cholinergic receptor, nicotinic, alpha 3]; CHRNA4 [cholinergic receptor, nicotinic, alpha 4]; CHRNA7 [cholinergic receptor, nicotinic, alpha 7]; CHUK [conserved helix-loop-helix ubiquitous kinase]; CIB1 [calcium and integrin binding 1 (calmyrin)]; CIITA [class II, major histocompatibility complex, transactivator]; CILP [cartilage intermediate layer protein, nucleotide pyrophosphohydrolase]; CISH [cytokine inducible SH2-containing protein]; CKB [creatine kinase, brain]; CKLF [chemokine-like factor]; CKM [creatine kinase, muscle]; CLC [Charcot-Leyden crystal protein]; CLCA1 [chloride channel accessory 1]; CLCN1 [chloride channel 1, skeletal muscle]; CLCN3 [chloride channel 3]; CLDN1 [claudin 1]; CLDN11 [claudin 11]; CLDN14 [claudin 14]; CLDN16 [claudin 16]; CLDN19 [claudin 19]; CLDN2 [claudin 2]; CLDN3 [claudin 3]; CLDN4 [claudin 4]; CLDN5 [claudin 5]; CLDN7 [claudin 7]; CLDN8 [claudin 8]; CLEC12A [C-type lectin domain family 12, member A]; CLEC16A [C-type lectin domain family 16, member A]; CLEC4A [C-type lectin domain family 4, member A]; CLEC4D [C-type lectin domain family 4, member D]; CLEC4M [C-type lectin domain family 4, member M]; CLEC7A [C-type lectin domain family 7, member A]; CLIP2 [CAP-GLY domain containing linker protein 2]; CLK2 [CDC-like kinase 2]; CLSPN [claspin homolog (Xenopus laevis)]; CLSTN2 [calsyntenin 2]; CLTCL1 [clathrin, heavy chain-like 1]; CLU [clusterin]; CMA1 [chymase 1, mast cell]; CMKLR1 [chemokine-like receptor 1]; CNBP [CCHC-type zinc finger, nucleic acid binding protein]; CNDP2 [CNDP dipeptidase 2 (metallopeptidase M20 family)]; CNN1 [calponin 1, basic, smooth muscle]; CNP [2′,3′-cyclic nucleotide 3′ phosphodiesterase]; CNR1 [cannabinoid receptor 1 (brain)]; CNR2 [cannabinoid receptor 2 (macrophage)]; CNTF [ciliary neurotrophic factor]; CNTN2 [contactin 2 (axonal)]; COG1 [component of oligomeric golgi complex 1]; COG2 [component of oligomeric golgi complex 2]; COIL [coilin]; COL11A1 [collagen, type XI, alpha 1]; COL11A2 [collagen, type XI, alpha 2]; COL17A1 [collagen, type XVII, alpha 1]; COL18A1 [collagen, type XVIII, alpha 1]; COL1A1 [collagen, type I, alpha 1]; COL1A2 [collagen, type I, alpha 2]; COL2A1 [collagen, type II, alpha 1]; COL3A1 [collagen, type III, alpha 1]; COL4A1 [collagen, type IV, alpha 1]; COL4A3 [collagen, type IV, alpha 3 (Goodpasture antigen)]; COL4A4 [collagen, type IV, alpha 4]; COL4A5 [collagen, type IV, alpha 5]; COL4A6 [collagen, type IV, alpha 6]; COL5A1 [collagen, type V, alpha 1]; COL5A2 [collagen, type V, alpha 2]; COL6A1 [collagen, type VI, alpha 1]; COL6A2 [collagen, type VI, alpha 2]; COL6A3 [collagen, type VI, alpha 3]; COL7A1 [collagen, type VII, alpha 1]; COL8A2 [collagen, type VIII, alpha 2]; COL9A1 [collagen, type IX, alpha 1]; COMT [catechol-O-methyltransferase]; COQ3 [coenzyme Q3 homolog, methyltransferase (S. cerevisiae)]; COQ7 [coenzyme Q7 homolog, ubiquinone (yeast)]; CORO1A [coronin, actin binding protein, 1A]; COX10 [COX10 homolog, cytochrome c oxidase assembly protein, heme A: farnesyltransferase (yeast)]; COX15 [COX15 homolog, cytochrome c oxidase assembly protein (yeast)]; COX5A [cytochrome c oxidase subunit Va]; COX8A [cytochrome c oxidase subunit VIIIA (ubiquitous)]; CP [ceruloplasmin (ferroxidase)]; CPA1 [carboxypeptidase A1 (pancreatic)]; CPB2 [carboxypeptidase B2 (plasma)]; CPN1 [carboxypeptidase N, polypeptide 1]; CPOX [coproporphyrinogen oxidase]; CPS1 [carbamoyl-phosphate synthetase 1, mitochondrial]; CPT2 [carnitine palmitoyltransferase 2]; CR1 [complement component (3b/4b) receptor 1 (Knops blood group)]; CR2 [complement component (3d/Epstein Barr virus) receptor 2]; CRAT [carnitine O-acetyltransferase]; CRB1 [crumbs homolog 1 (Drosophila)]; CREB1 [cAMP responsive element binding protein 1]; CREBBP [CREB binding protein]; CREM [cAMP responsive element modulator]; CRH [corticotropin releasing hormone]; CRHR1 [corticotropin releasing hormone receptor 1]; CRHR2 [corticotropin releasing hormone receptor 2]; CRK [v-crk sarcoma virus CT10 oncogene homolog (avian)]; CRKL [v-crk sarcoma virus CT10 oncogene homolog (avian)-like]; CRLF2 [cytokine receptor-like factor 2]; CRLF3 [cytokine receptor-like factor 3]; CROT [carnitine O-octanoyltransferase]; CRP [C-reactive protein, pentraxin-related]; CRX [cone-rod homeobox]; CRY2 [cryptochrome 2 (photolyase-like)]; CRYAA [crystallin, alpha A]; CRYAB [crystallin, alpha B]; CS [citrate synthase]; CSF1 [colony stimulating factor 1 (macrophage)]; CSF1R [colony stimulating factor 1 receptor]; CSF2 [colony stimulating factor 2 (granulocyte-macrophage)]; CSF2RB [colony stimulating factor 2 receptor, beta, low-affinity (granulocyte-macrophage)]; CSF3 [colony stimulating factor 3 (granulocyte)]; CSF3R [colony stimulating factor 3 receptor (granulocyte)]; CSK [c-src tyrosine kinase]; CSMD3 [CUB and Sushi multiple domains 3]; CSN1S1 [casein alpha s1]; CSN2 [casein beta]; CSNK1A1 [casein kinase 1, alpha 1]; CSNK2A1 [casein kinase 2, alpha 1 polypeptide]; CSNK2B [casein kinase 2, beta polypeptide]; CSPG4 [chondroitin sulfate proteoglycan 4]; CST3 [cystatin C]; CST8 [cystatin 8 (cystatin-related epididymal specific)]; CSTA [cystatin A (stefin A)]; CSTB [cystatin B (stefin B)]; CTAGE1 [cutaneous T-cell lymphoma-associated antigen 1]; CTF1 [cardiotrophin 1]; CTGF [connective tissue growth factor]; CTH [cystathionase (cystathionine gamma-lyase)]; CTLA4 [cytotoxic T-lymphocyte-associated protein 4]; CTNNA1 [catenin (cadherin-associated protein), alpha 1, 102 kDa]; CTNNA3 [catenin (cadherin-associated protein), alpha 3]; CTNNAL1 [catenin (cadherin-associated protein), alpha-like 1]; CTNNB1 [catenin (cadherin-associated protein), beta 1, 88 kDa]; CTNND1 [catenin (cadherin-associated protein), delta 1]; CTNS [cystinosis, nephropathic]; CTRL [chymotrypsin-like]; CTSB [cathepsin B]; CTSC [cathepsin C]; CTSD [cathepsin D]; CTSE [cathepsin E]; CTSG [cathepsin G]; CTSH [cathepsin H]; CTSK [cathepsin K]; CTSL1 [cathepsin L1]; CTTN [cortactin]; CUL1 [cullin 1]; CUL2 [cullin 2]; CUL4A [cullin 4A]; CUL5 [cullin 5]; CX3CL1 [chemokine (C-X3-C motif) ligand 1]; CX3CR1 [chemokine (C-X3-C motif) receptor 1]; CXADR [coxsackie virus and adenovirus receptor]; CXCL1 [chemokine (C-X-C motif) ligand 1 (melanoma growth stimulating activity, alpha)]; CXCL10 [chemokine (C-X-C motif) ligand 10]; CXCL11 [chemokine (C-X-C motif) ligand 11]; CXCL12 [chemokine (C-X-C motif) ligand 12 (stromal cell-derived factor 1)]; CXCL13 [chemokine (C-X-C motif) ligand 13]; CXCL2 [chemokine (C-X-C motif) ligand 2]; CXCL5 [chemokine (C-X-C motif) ligand 5]; CXCL6 [chemokine (C-X-C motif) ligand 6 (granulocyte chemotactic protein 2)]; CXCL9 [chemokine (C-X-C motif) ligand 9]; CXCR1 [chemokine (C-X-C motif) receptor 1]; CXCR2 [chemokine (C-X-C motif) receptor 2]; CXCR3 [chemokine (C-X-C motif) receptor 3]; CXCR4 [chemokine (C-X-C motif) receptor 4]; CXCR5 [chemokine (C-X-C motif) receptor 5]; CXCR6 [chemokine (C-X-C motif) receptor 6]; CXCR7 [chemokine (C-X-C motif) receptor 7]; CXorf40A [chromosome X open reading frame 40A]; CYB5A [cytochrome b5 type A (microsomal)]; CYB5R3 [cytochrome b5 reductase 3]; CYBA [cytochrome b-245, alpha polypeptide]; CYBB [cytochrome b-245, beta polypeptide]; CYC1 [cytochrome c-1]; CYCS [cytochrome c, somatic]; CYFIP2 [cytoplasmic FMR1 interacting protein 2]; CYP11A1 [cytochrome P450, family 11, subfamily A, polypeptide 1]; CYP11B1 [cytochrome P450, family 11, subfamily B, polypeptide 1]; CYP11B2 [cytochrome P450, family 11, subfamily B, polypeptide 2]; CYP17A1 [cytochrome P450, family 17, subfamily A, polypeptide 1]; CYP19A1 [cytochrome P450, family 19, subfamily A, polypeptide 1]; CYP1A1 [cytochrome P450, family 1, subfamily A, polypeptide 1]; CYP1A2 [cytochrome P450, family 1, subfamily A, polypeptide 2]; CYP1B1 [cytochrome P450, family 1, subfamily B, polypeptide 1]; CYP21A2 [cytochrome P450, family 21, subfamily A, polypeptide 2]; CYP24A1 [cytochrome P450, family 24, subfamily A, polypeptide 1]; CYP27A1 [cytochrome P450, family 27, subfamily A, polypeptide 1]; CYP27B1 [cytochrome P450, family 27, subfamily B, polypeptide 1]; CYP2A6 [cytochrome P450, family 2, subfamily A, polypeptide 6]; CYP2B6 [cytochrome P450, family 2, subfamily B, polypeptide 6]; CYP2C19 [cytochrome P450, family 2, subfamily C, polypeptide 19]; CYP2C8 [cytochrome P450, family 2, subfamily C, polypeptide 8]; CYP2C9 [cytochrome P450, family 2, subfamily C, polypeptide 9]; CYP2D6 [cytochrome P450, family 2, subfamily D, polypeptide 6]; CYP2E1 [cytochrome P450, family 2, subfamily E, polypeptide 1]; CYP2J2 [cytochrome P450, family 2, subfamily J, polypeptide 2]; CYP2R1 [cytochrome P450, family 2, subfamily R, polypeptide 1]; CYP3A4 [cytochrome P450, family 3, subfamily A, polypeptide 4]; CYP3A5 [cytochrome P450, family 3, subfamily A, polypeptide 5]; CYP4F3 [cytochrome P450, family 4, subfamily F, polypeptide 3]; CYP51A1 [cytochrome P450, family 51, subfamily A, polypeptide 1]; CYP7A1 [cytochrome P450, family 7, subfamily A, polypeptide 1]; CYR61 [cysteine-rich, angiogenic inducer, 61]; CYSLTR1 [cysteinyl leukotriene receptor 1]; CYSLTR2 [cysteinyl leukotriene receptor 2]; DAO [D-amino-acid oxidase]; DAOA [D-amino acid oxidase activator]; DAP3 [death associated protein 3]; DAPK1 [death-associated protein kinase 1]; DARC [Duffy blood group, chemokine receptor]; DAZ1 [deleted in azoospermia 1]; DBH [dopamine beta-hydroxylase (dopamine beta-monooxygenase)]; DCK [deoxycytidine kinase]; DCLRE1C [DNA cross-link repair 1C (PSO2 homolog, S. cerevisiae)]; DCN [decorin]; DCT [dopachrome tautomerase (dopachrome delta-isomerase, tyrosine-related protein 2)]; DCTN2 [dynactin 2 (p50)]; DDB1 [damage-specific DNA binding protein 1, 127 kDa]; DDB2 [damage-specific DNA binding protein 2, 48 kDa]; DDC [dopa decarboxylase (aromatic L-amino acid decarboxylase)]; DDIT3 [DNA-damage-inducible transcript 3]; DDR1 [discoidin domain receptor tyrosine kinase 1]; DDX1 [DEAD (Asp-Glu-Ala-Asp) box polypeptide 1]; DDX41 [DEAD (Asp-Glu-Ala-Asp) box polypeptide 41]; DDX42 [DEAD (Asp-Glu-Ala-Asp) box polypeptide 42]; DDX58 [DEAD (Asp-Glu-Ala-Asp) box polypeptide 58]; DEFA1 [defensin, alpha 1]; DEFAS [defensin, alpha 5, Paneth cell-specific]; DEFA6 [defensin, alpha 6, Paneth cell-specific]; DEFB1 [defensin, beta 1]; DEFB103B [defensin, beta 103B]; DEFB104A [defensin, beta 104A]; DEFB4A [defensin, beta 4A]; DEK [DEK oncogene]; DENND1B [DENN/MADD domain containing 1B]; DES [desmin]; DGAT1 [diacylglycerol O-acyltransferase homolog 1 (mouse)]; DGCR14 [DiGeorge syndrome critical region gene 14]; DGCR2 [DiGeorge syndrome critical region gene 2]; DGCR6 [DiGeorge syndrome critical region gene 6]; DGCR6L [DiGeorge syndrome critical region gene 6-like]; DGCR8 [DiGeorge syndrome critical region gene 8]; DGUOK [deoxyguanosine kinase]; DHFR [dihydrofolate reductase]; DHODH [dihydroorotate dehydrogenase]; DHPS [deoxyhypusine synthase]; DHRS7B [dehydrogenase/reductase (SDR family) member 7B]; DHRS9 [dehydrogenase/reductase (SDR family) member 9]; DIAPH1 [diaphanous homolog 1 (Drosophila)]; DICER1 [dicer 1, ribonuclease type III]; DIO2 [deiodinase, iodothyronine, type II]; DKC1 [dyskeratosis congenita 1, dyskerin]; DKK1 [dickkopf homolog 1 (Xenopus laevis)]; DLAT [dihydrolipoamide S-acetyltransferase]; DLG2 [discs, large homolog 2 (Drosophila)]; DLG5 [discs, large homolog 5 (Drosophila)]; DMBT1 [deleted in malignant brain tumors 1]; DMC1 [DMC1 dosage suppressor of mck1 homolog, meiosis-specific homologous recombination (yeast)]; DMD [dystrophin]; DMP1 [dentin matrix acidic phosphoprotein 1]; DMPK [dystrophia myotonica-protein kinase]; DMRT1 [doublesex and mab-3 related transcription factor 1]; DMXL2 [Dmx-like 2]; DNA2 [DNA replication helicase 2 homolog (yeast)]; DNAH1 [dynein, axonemal, heavy chain 1]; DNAH12 [dynein, axonemal, heavy chain 12]; DNAI1 [dynein, axonemal, intermediate chain 1]; DNAI2 [dynein, axonemal, intermediate chain 2]; DNASE1 [deoxyribonuclease I]; DNM2 [dynamin 2]; DNM3 [dynamin 3]; DNMT1 [DNA (cytosine-5-)-methyltransferase 1]; DNMT3B [DNA (cytosine-5-)-methyltransferase 3 beta]; DNTT [deoxynucleotidyltransferase, terminal]; DOCK1 [dedicator of cytokinesis 1]; DOCK3 [dedicator of cytokinesis 3]; DOCK8 [dedicator of cytokinesis 8]; DOK1 [docking protein 1, 62 kDa (downstream of tyrosine kinase 1)]; DOLK [dolichol kinase]; DPAGT1 [dolichyl-phosphate (UDP-N-acetylglucosamine) N-acetylglucosaminephosphotransferase 1 (GlcNAc-1-P transferase)]; DPEP1 [dipeptidase 1 (renal)]; DPH1 [DPH1 homolog (S. cerevisiae)]; DPM1 [dolichyl-phosphate mannosyltransferase polypeptide 1, catalytic subunit]; DPP10 [dipeptidyl-peptidase 10]; DPP4 [dipeptidyl-peptidase 4]; DPYD [dihydropyrimidine dehydrogenase]; DRD2 [dopamine receptor D2]; DRD3 [dopamine receptor D3]; DRD4 [dopamine receptor D4]; DSC2 [desmocollin 2]; DSG1 [desmoglein 1]; DSG2 [desmoglein 2]; DSG3 [desmoglein 3 (pemphigus vulgaris antigen)]; DSP [desmoplakin]; DTNA [dystrobrevin, alpha]; DTYMK [deoxythymidylate kinase (thymidylate kinase)]; DUOX1 [dual oxidase 1]; DUOX2 [dual oxidase 2]; DUSP1 [dual specificity phosphatase 1]; DUSP14 [dual specificity phosphatase 14]; DUSP2 [dual specificity phosphatase 2]; DUSP5 [dual specificity phosphatase 5]; DUT [deoxyuridine triphosphatase]; DVL1 [dishevelled, dsh homolog 1 (Drosophila)]; DYNC2H1 [dynein, cytoplasmic 2, heavy chain 1]; DYNLL1 [dynein, light chain, LC8-type 1]; DYRK1A [dual-specificity tyrosine-(Y)-phosphorylation regulated kinase 1A]; DYSF [dysferlin, limb girdle muscular dystrophy 2B (autosomal recessive)]; E2F1 [E2F transcription factor 1]; EBF2 [early B-cell factor 2]; EBI3 [Epstein-Barr virus induced 3]; ECE1 [endothelin converting enzyme 1]; ECM1 [extracellular matrix protein 1]; EDA [ectodysplasin A]; EDAR [ectodysplasin A receptor]; EDN1 [endothelin 1]; EDNRA [endothelin receptor type A]; EDNRB [endothelin receptor type B]; EEF1A1 [eukaryotic translation elongation factor 1 alpha 1]; EEF1A2 [eukaryotic translation elongation factor 1 alpha 2]; EFEMP2 [EGF-containing fibulin-like extracellular matrix protein 2]; EFNA1 [ephrin-A1]; EFNB2 [ephrin-B2]; EFS [embryonal Fyn-associated substrate]; EGF [epidermal growth factor (beta-urogastrone)]; EGFR [epidermal growth factor receptor (erythroblastic leukemia viral (v-erb-b) oncogene homolog, avian)]; EGR1 [early growth response 1]; EGR2 [early growth response 2]; EHF [ets homologous factor]; EHMT2 [euchromatic histone-lysine N-methyltransferase 2]; EIF2AK2 [eukaryotic translation initiation factor 2-alpha kinase 2]; EIF2S1 [eukaryotic translation initiation factor 2, subunit 1 alpha, 35 kDa]; EIF2S2 [eukaryotic translation initiation factor 2, subunit 2 beta, 38 kDa]; EIF3A [eukaryotic translation initiation factor 3, subunit A]; EIF4B [eukaryotic translation initiation factor 4B]; EIF4E [eukaryotic translation initiation factor 4E]; EIF4EBP1 [eukaryotic translation initiation factor 4E binding protein 1]; EIF4G1 [eukaryotic translation initiation factor 4 gamma, 1]; EIF6 [eukaryotic translation initiation factor 6]; ELAC2 [elaC homolog 2 (E. coli)]; ELANE [elastase, neutrophil expressed]; ELAVL1 [ELAV (embryonic lethal, abnormal vision, Drosophila)-like 1 (Hu antigen R)]; ELF3 [E74-like factor 3 (ets domain transcription factor, epithelial-specific)]; ELF5 [E74-like factor 5 (ets domain transcription factor)]; ELN [elastin]; ELOVL4 [elongation of very long chain fatty acids (FEN1/Elo2, SUR4/Elo3, yeast)-like 4]; EMD [emerin]; EMILIN1 [elastin microfibril interfacer 1]; EMR2 [egf-like module containing, mucin-like, hormone receptor-like 2]; EN2 [engrailed homeobox 2]; ENG [endoglin]; ENO1 [enolase 1, (alpha)]; ENO2 [enolase 2 (gamma, neuronal)]; ENO3 [enolase 3 (beta, muscle)]; ENPP2 [ectonucleotide pyrophosphatase/phosphodiesterase 2]; ENPP3 [ectonucleotide pyrophosphatase/phosphodiesterase 3]; ENTPD1 [ectonucleoside triphosphate diphosphohydrolase 1]; EP300 [E1A binding protein p300]; EPAS1 [endothelial PAS domain protein 1]; EPB42 [erythrocyte membrane protein band 4.2]; EPCAM [epithelial cell adhesion molecule]; EPHA1 [EPH receptor A1]; EPHA2 [EPH receptor A2]; EPHB2 [EPH receptor B2]; EPHB4 [EPH receptor B4]; EPHB6 [EPH receptor B6]; EPHX1 [epoxide hydrolase 1, microsomal (xenobiotic)]; EPHX2 [epoxide hydrolase 2, cytoplasmic]; EPO [erythropoietin]; EPOR [erythropoietin receptor]; EPRS [glutamyl-prolyl-tRNA synthetase]; EPX [eosinophil peroxidase]; ERBB2 [v-erb-b2 erythroblastic leukemia viral oncogene homolog 2, neuro/glioblastoma derived oncogene homolog (avian)]; ERBB21P [erbb2 interacting protein]; ERBB3 [v-erb-b2 erythroblastic leukemia viral oncogene homolog 3 (avian)]; ERBB4 [v-erb-a erythroblastic leukemia viral oncogene homolog 4 (avian)]; ERCC1 [excision repair cross-complementing rodent repair deficiency, complementation group 1 (includes overlapping antisense sequence)]; ERCC2 [excision repair cross-complementing rodent repair deficiency, complementation group 2]; ERCC3 [excision repair cross-complementing rodent repair deficiency, complementation group 3 (xeroderma pigmentosum group B complementing)]; ERCC4 [excision repair cross-complementing rodent repair deficiency, complementation group 4]; ERCC5 [excision repair cross-complementing rodent repair deficiency, complementation group 5]; ERCC6 [excision repair cross-complementing rodent repair deficiency, complementation group 6]; ERCC6L [excision repair cross-complementing rodent repair deficiency, complementation group 6-like]; ERCC8 [excision repair cross-complementing rodent repair deficiency, complementation group 8]; ERO1LB [ERO1-like beta (S. cerevisiae)]; ERVK6 [endogenous retroviral sequence K, 6]; ERVWE1 [endogenous retroviral family W, env(C7), member 1]; ESD [esterase D/formylglutathione hydrolase]; ESR1 [estrogen receptor 1]; ESR2 [estrogen receptor 2 (ER beta)]; ESRRA [estrogen-related receptor alpha]; ESRRB [estrogen-related receptor beta]; ETS1 [v-ets erythroblastosis virus E26 oncogene homolog 1 (avian)]; ETS2 [v-ets erythroblastosis virus E26 oncogene homolog 2 (avian)]; EWSR1 [Ewing sarcoma breakpoint region 1]; EXO1 [exonuclease 1]; EYA1 [eyes absent homolog 1 (Drosophila)]; EZH2 [enhancer of zeste homolog 2 (Drosophila)]; EZR [ezrin]; F10 [coagulation factor X]; F11 [coagulation factor XI]; F12 [coagulation factor XII (Hageman factor)]; F13A1 [coagulation factor XIII, A1 polypeptide]; F13B [coagulation factor XIII, B polypeptide]; F2 [coagulation factor II (thrombin)]; F2R [coagulation factor II (thrombin) receptor]; F2RL1 [coagulation factor II (thrombin) receptor-like 1]; F2RL3 [coagulation factor II (thrombin) receptor-like 3]; F3 [coagulation factor III (thromboplastin, tissue factor)]; F5 [coagulation factor V (proaccelerin, labile factor)]; F7 [coagulation factor VII (serum prothrombin conversion accelerator)]; F8 [coagulation factor VIII, procoagulant component]; F9 [coagulation factor IX]; FABP1 [fatty acid binding protein 1, liver]; FABP2 [fatty acid binding protein 2, intestinal]; FABP4 [fatty acid binding protein 4, adipocyte]; FADD [Fas (TNFRSF6)-associated via death domain]; FADS1 [fatty acid desaturase 1]; FADS2 [fatty acid desaturase 2]; FAF1 [Fas (TNFRSF6) associated factor 1]; FAH [fumarylacetoacetate hydrolase (fumarylacetoacetase)]; FAM189B [family with sequence similarity 189, member B]; FAM92B [family with sequence similarity 92, member B]; FANCA [Fanconi anemia, complementation group A]; FANCB [Fanconi anemia, complementation group B]; FANCC [Fanconi anemia, complementation group C]; FANCD2 [Fanconi anemia, complementation group D2]; FANCE [Fanconi anemia, complementation group E]; FANCF [Fanconi anemia, complementation group F]; FANCG [Fanconi anemia, complementation group G]; FANGI [Fanconi anemia, complementation group I]; FANCL [Fanconi anemia, complementation group L]; FANCM [Fanconi anemia, complementation group M]; FANK1 [fibronectin type III and ankyrin repeat domains 1]; FAS [Fas (TNF receptor superfamily, member 6)]; FASLG [Fas ligand (TNF superfamily, member 6)]; FASN [fatty acid synthase]; FASTK [Fas-activated serine/threonine kinase]; FBLN5 [fibulin 5]; FBN1 [fibrillin 1]; FBP1 [fructose-1,6-bisphosphatase 1]; FBXO32 [F-box protein 32]; FBXW7 [F-box and WD repeat domain containing 7]; FCAR [Fc fragment of IgA, receptor for]; FCER1A [Fc fragment of IgE, high affinity I, receptor for; alpha polypeptide]; FCER1G [Fc fragment of IgE, high affinity I, receptor for; gamma polypeptide]; FCER2 [Fc fragment of IgE, low affinity II, receptor for (CD23)]; FCGR1A [Fc fragment of IgG, high affinity Ia, receptor (CD64)]; FCGR2A [Fc fragment of IgG, low affinity IIa, receptor (CD32)]; FCGR2B [Fc fragment of IgG, low affinity 11b, receptor (CD32)]; FCGR3A [Fc fragment of IgG, low affinity IIIa, receptor (CD16a)]; FCGR3B [Fc fragment of IgG, low affinity IIIb, receptor (CD16b)]; FCN2 [ficolin (collagen/fibrinogen domain containing lectin) 2 (hucolin)]; FCN3 [ficolin (collagen/fibrinogen domain containing) 3 (Hakata antigen)]; FCRL3 [Fc receptor-like 3]; FCRL6 [Fc receptor-like 6]; FDFT1 [farnesyl-diphosphate farnesyltransferase 1]; FDPS [farnesyl diphosphate synthase (farnesyl pyrophosphate synthetase, dimethylallyltranstransferase, geranyltranstransferase)]; FDX1 [ferredoxin 1]; FEN1 [flap structure-specific endonuclease 1]; FERMT1 [fermitin family homolog 1 (Drosophila)]; FERMT3 [fermitin family homolog 3 (Drosophila)]; FES [feline sarcoma oncogene]; FFAR2 [free fatty acid receptor 2]; FGA [fibrinogen alpha chain]; FGB [fibrinogen beta chain]; FGF1 [fibroblast growth factor 1 (acidic)]; FGF2 [fibroblast growth factor 2 (basic)]; FGF5 [fibroblast growth factor 5]; FGF7 [fibroblast growth factor 7 (keratinocyte growth factor)]; FGF8 [fibroblast growth factor 8 (androgen-induced)]; FGFBP2 [fibroblast growth factor binding protein 2]; FGFR1 [fibroblast growth factor receptor 1]; FGFR1OP [FGFR1 oncogene partner]; FGFR2 [fibroblast growth factor receptor 2]; FGFR3 [fibroblast growth factor receptor 3]; FGFR4 [fibroblast growth factor receptor 4]; FGG [fibrinogen gamma chain]; FGR [Gardner-Rasheed feline sarcoma viral (v-fgr) oncogene homolog]; FHIT [fragile histidine triad gene]; FHL1 [four and a half LIM domains 1]; FHL2 [four and a half LIM domains 2]; FIBP [fibroblast growth factor (acidic) intracellular binding protein]; FIGF [c-fos induced growth factor (vascular endothelial growth factor D)]; FKBP1A [FK506 binding protein 1A, 12 kDa]; FKBP4 [FK506 binding protein 4, 59 kDa]; FKBP5 [FK506 binding protein 5]; FLCN [folliculin]; FLG [filaggrin]; FLG2 [filaggrin family member 2]; FLNA [filamin A, alpha]; FLNB [filamin B, beta]; FLT1 [fms-related tyrosine kinase 1 (vascular endothelial growth factor/vascular permeability factor receptor)]; FLT3 [fms-related tyrosine kinase 3]; FLT3LG [fms-related tyrosine kinase 3 ligand]; FLT4 [fms-related tyrosine kinase 4]; FMN1 [formin 1]; FMOD [fibromodulin]; FMR1 [fragile X mental retardation 1]; FN1 [fibronectin 1]; FOLH1 [folate hydrolase (prostate-specific membrane antigen) 1]; FOLR1 [folate receptor 1 (adult)]; FOS [FBJ murine osteosarcoma viral oncogene homolog]; FOXL2 [forkhead box L2]; FOXN1 [forkhead box N1]; FOXN2 [forkhead box N2]; FOXO3 [forkhead box 03]; FOXP3 [forkhead box P3]; FPGS [folylpolyglutamate synthase]; FPR1 [formyl peptide receptor 1]; FPR2 [formyl peptide receptor 2]; FRAS1 [Fraser syndrome 1]; FREM2 [FRAS1 related extracellular matrix protein 2]; FSCN1 [fascin homolog 1, actin-bundling protein (Strongylocentrotus purpuratus)]; FSHB [follicle stimulating hormone, beta polypeptide]; FSHR [follicle stimulating hormone receptor]; FST [follistatin]; FTCD [formiminotransferase cyclodeaminase]; FTH1 [ferritin, heavy polypeptide 1]; FTL [ferritin, light polypeptide]; FURIN [furin (paired basic amino acid cleaving enzyme)]; FUT1 [fucosyltransferase 1 (galactoside 2-alpha-L-fucosyltransferase, H blood group)]; FUT2 [fucosyltransferase 2 (secretor status included)]; FUT3 [fucosyltransferase 3 (galactoside 3(4)-L-fucosyltransferase, Lewis blood group)]; FUT4 [fucosyltransferase 4 (alpha (1,3) fucosyltransferase, myeloid-specific)]; FUT7 [fucosyltransferase 7 (alpha (1,3) fucosyltransferase)]; FUT8 [fucosyltransferase 8 (alpha (1,6) fucosyltransferase)]; FXN [frataxin]; FYN [FYN oncogene related to SRC, FGR, YES]; FZD4 [frizzled homolog 4 (Drosophila)]; G6PC3 [glucose 6 phosphatase, catalytic, 3]; G6PD [glucose-6-phosphate dehydrogenase]; GAA [glucosidase, alpha; acid]; GAB2 [GRB2-associated binding protein 2]; GABBR1 [gamma-aminobutyric acid (GABA) B receptor, 1]; GABRB3 [gamma-aminobutyric acid (GABA) A receptor, beta 3]; GABRE [gamma-aminobutyric acid (GABA) A receptor, epsilon]; GAD1 [glutamate decarboxylase 1 (brain, 67 kDa)]; GAD2 [glutamate decarboxylase 2 (pancreatic islets and brain, 65 kDa)]; GADD45A [growth arrest and DNA-damage-inducible, alpha]; GAL [galanin prepropeptide]; GALC [galactosylceramidase]; GALK1 [galactokinase 1]; GALR1 [galanin receptor 1]; GAP43 [growth associated protein 43]; GAPDH [glyceraldehyde-3-phosphate dehydrogenase]; GART [phosphoribosylglycinamide formyltransferase, phosphoribosylglycinamide synthetase, phosphoribosylaminoimidazole synthetase]; GAST [gastrin]; GATA1 [GATA binding protein 1 (globin transcription factor 1)]; GATA2 [GATA binding protein 2]; GATA3 [GATA binding protein 3]; GATA4 [GATA binding protein 4]; GATA6 [GATA binding protein 6]; GBA [glucosidase, beta, acid]; GBA3 [glucosidase, beta, acid 3 (cytosolic)]; GBE1 [glucan (1 [4-alpha-), branching enzyme 1]; GC [group-specific component (vitamin D binding protein)]; GCG [glucagon]; GCH1 [GTP cyclohydrolase 1]; GCKR [glucokinase (hexokinase 4) regulator]; GCLC [glutamate-cysteine ligase, catalytic subunit]; GCLM [glutamate-cysteine ligase, modifier subunit]; GCNT2 [glucosaminyl (N-acetyl) transferase 2, 1-branching enzyme (I blood group)]; GDAP1 [ganglioside-induced differentiation-associated protein 1]; GDF15 [growth differentiation factor 15]; GDNF [glial cell derived neurotrophic factor]; GFAP [glial fibrillary acidic protein]; GGH [gamma-glutamyl hydrolase (conjugase, folylpolygammaglutamyl hydrolase)]; GGT1 [gamma-glutamyltransferase 1]; GGT2 [gamma-glutamyltransferase 2]; GH1 [growth hormone 1]; GHR [growth hormone receptor]; GHRH [growth hormone releasing hormone]; GHRL [ghrelin/obestatin prepropeptide]; GHSR [growth hormone secretagogue receptor]; GIF [gastric intrinsic factor (vitamin B synthesis)]; GIP [gastric inhibitory polypeptide]; GJA1 [gap junction protein, alpha 1, 43 kDa]; GJA4 [gap junction protein, alpha 4, 37 kDa]; GJB2 [gap junction protein, beta 2, 26 kDa]; GLA [galactosidase, alpha]; GLB1 [galactosidase, beta 1]; GLI2 [GLI family zinc finger 2]; GLMN [glomulin, FKBP associated protein]; GLX [glutaredoxin (thioltransferase)]; GLS [glutaminase]; GLT25D1 [glycosyltransferase 25 domain containing 1]; GLUL [glutamate-ammonia ligase (glutamine synthetase)]; GLYAT [glycine-N-acyltransferase]; GM2A [GM2 ganglioside activator]; GMDS [GDP-mannose 4 [6-dehydratase]; GNA12 [guanine nucleotide binding protein (G protein) alpha 12]; GNA13 [guanine nucleotide binding protein (G protein), alpha 13]; GNAI1 [guanine nucleotide binding protein (G protein), alpha inhibiting activity polypeptide 1]; GNAO1 [guanine nucleotide binding protein (G protein), alpha activating activity polypeptide 0]; GNAQ [guanine nucleotide binding protein (G protein), q polypeptide]; GNAS [GNAS complex locus]; GNAZ [guanine nucleotide binding protein (G protein), alpha z polypeptide]; GNB1 [guanine nucleotide binding protein (G protein), beta polypeptide 1]; GNB1L [guanine nucleotide binding protein (G protein), beta polypeptide 1-like]; GNB2L1 [guanine nucleotide binding protein (G protein), beta polypeptide 2-like 1]; GNB3 [guanine nucleotide binding protein (G protein), beta polypeptide 3]; GNE [glucosamine (UDP-N-acetyl)-2-epimerase/N-acetylmannosamine kinase]; GNG2 [guanine nucleotide binding protein (G protein), gamma 2]; GNLY [granulysin]; GNPAT [glyceronephosphate O-acyltransferase]; GNPDA2 [glucosamine-6-phosphate deaminase 2]; GNRH1 [gonadotropin-releasing hormone 1 (luteinizing-releasing hormone)]; GNRHR [gonadotropin-releasing hormone receptor]; GOLGA8B [golgin A8 family, member B]; GOLGB1 [golgin B1]; GOT1 [glutamic-oxaloacetic transaminase 1, soluble (aspartate aminotransferase 1)]; GOT2 [glutamic-oxaloacetic transaminase 2, mitochondrial (aspartate aminotransferase 2)]; GP1BA [glycoprotein Ib (platelet), alpha polypeptide]; GP2 [glycoprotein 2 (zymogen granule membrane)]; GP6 [glycoprotein VI (platelet)]; GPBAR1 [G protein-coupled bile acid receptor 1]; GPC5 [glypican 5]; GPI [glucose phosphate isomerase]; GPLD1 [glycosylphosphatidylinositol specific phospholipase D1]; GPN1 [GPN-loop GTPase 1]; GPR1 [G protein-coupled receptor 1]; GPR12 [G protein-coupled receptor 12]; GPR123 [G protein-coupled receptor 123]; GPR143 [G protein-coupled receptor 143]; GPR15 [G protein-coupled receptor 15]; GPR182 [G protein-coupled receptor 182]; GPR44 [G protein-coupled receptor 44]; GPR77 [G protein-coupled receptor 77]; GPRASP1 [G protein-coupled receptor associated sorting protein 1]; GPRC6A [G protein-coupled receptor, family C, group 6, member A]; GPT [glutamic-pyruvate transaminase (alanine aminotransferase)]; GPX1 [glutathione peroxidase 1]; GPX2 [glutathione peroxidase 2 (gastrointestinal)]; GPX3 [glutathione peroxidase 3 (plasma)]; GRAP2 [GRB2-related adaptor protein 2]; GRB2 [growth factor receptor-bound protein 2]; GRIA2 [glutamate receptor, ionotropic, AMPA 2]; GRIN1 [glutamate receptor, ionotropic, N-methyl D-aspartate 1]; GRIN2A [glutamate receptor, ionotropic, N-methyl D-aspartate 2A]; GRIN2B [glutamate receptor, ionotropic, N-methyl D-aspartate 2B]; GRIN2C [glutamate receptor, ionotropic, N-methyl D-aspartate 20]; GRIN2D [glutamate receptor, ionotropic, N-methyl D-aspartate 2D]; GRIN3A [glutamate receptor, ionotropic, N-methyl-D-aspartate 3A]; GRIN3B [glutamate receptor, ionotropic, N-methyl-D-aspartate 3B]; GRK5 [G protein-coupled receptor kinase 5]; GRLF1 [glucocorticoid receptor DNA binding factor 1]; GRM1 [glutamate receptor, metabotropic 1]; GRP [gastrin-releasing peptide]; GRPR [gastrin-releasing peptide receptor]; GSC [goosecoid homeobox]; GSC2 [goosecoid homeobox 2]; GSDMB [gasdermin B]; GSK3B [glycogen synthase kinase 3 beta]; GSN [gelsolin]; GSR [glutathione reductase]; GSS [glutathione synthetase]; GSTA1 [glutathione S-transferase alpha 1]; GSTA2 [glutathione S-transferase alpha 2]; GSTM1 [glutathione S-transferase mu 1]; GSTM3 [glutathione S-transferase mu 3 (brain)]; GSTO2 [glutathione S-transferase omega 2]; GSTP1 [glutathione S-transferase pi 1]; GSTT1 [glutathione S-transferase theta 1]; GTF2A1 [general transcription factor IIA, 1, 19/37 kDa]; GTF2F1 [general transcription factor IIF, polypeptide 1, 74 kDa]; GTF2H2 [general transcription factor IIH, polypeptide 2, 44 kDa]; GTF2H4 [general transcription factor IIH, polypeptide 4, 52 kDa]; GTF2H5 [general transcription factor IIH, polypeptide 5]; GTF2I [general transcription factor IIi]; GTF3A [general transcription factor 111A]; GUCA2A [guanylate cyclase activator 2A (guanylin)]; GUCA2B [guanylate cyclase activator 2B (uroguanylin)]; GUCY2C [guanylate cyclase 2C (heat stable enterotoxin receptor)]; GUK1 [guanylate kinase 1]; GULP1 [GULP, engulfment adaptor PTB domain containing 1]; GUSB [glucuronidase, beta]; GYPA [glycophorin A (MNS blood group)]; GYPB [glycophorin B (MNS blood group)]; GYPC [glycophorin C (Gerbich blood group)]; GYPE [glycophorin E (MNS blood group)]; GYS1 [glycogen synthase 1 (muscle)]; GZMA [granzyme A (granzyme 1, cytotoxic T-lymphocyte-associated serine esterase 3)]; GZMB [granzyme B (granzyme 2, cytotoxic T-lymphocyte-associated serine esterase 1)]; GZMK [granzyme K (granzyme 3; tryptase II)]; H1F0 [H1 histone family, member 0]; H2AFX [H2A histone family, member X]; HABP2 [hyaluronan binding protein 2]; HACL1 [2-hydroxyacyl-CoA lyase 1]; HADHA [hydroxyacyl-Coenzyme A dehydrogenase/3-ketoacyl-Coenzyme A thiolase/enoyl-Coenzyme A hydratase (trifunctional protein), alpha subunit]; HAL [histidine ammonia-lyase]; HAMP [hepcidin antimicrobial peptide]; HAPLN1 [hyaluronan and proteoglycan link protein 1]; HAVCR1 [hepatitis A virus cellular receptor 1]; HAVCR2 [hepatitis A virus cellular receptor 2]; HAX1 [HCLS1 associated protein X-1]; HBA1 [hemoglobin, alpha 1]; HBA2 [hemoglobin, alpha 2]; HBB [hemoglobin, beta]; HBE1 [hemoglobin, epsilon 1]; HBEGF [heparin-binding EGF-like growth factor]; HBG2 [hemoglobin, gamma G]; HCCS [holocytochrome c synthase (cytochrome c heme-lyase)]; HCK [hemopoietic cell kinase]; HCRT [hypocretin (orexin) neuropeptide precursor]; HCRTR1 [hypocretin (orexin) receptor 1]; HCRTR2 [hypocretin (orexin) receptor 2]; HOST [hematopoietic cell signal transducer]; HDAC1 [histone deacetylase 1]; HDAC2 [histone deacetylase 2]; HDAC6 [histone deacetylase 6]; HDAC9 [histone deacetylase 9]; HDC [histidine decarboxylase]; HERC2 [hect domain and RLD 2]; HES1 [hairy and enhancer of split 1, (Drosophila)]; HES6 [hairy and enhancer of split 6 (Drosophila)]; HESX1 [HESX homeobox 1]; HEXA [hexosaminidase A (alpha polypeptide)]; HEXB [hexosaminidase B (beta polypeptide)]; HFE [hemochromatosis]; HGF [hepatocyte growth factor (hepapoietin A; scatter factor)]; HGS [hepatocyte growth factor-regulated tyrosine kinase substrate]; HGSNAT [heparan-alpha-glucosaminide N-acetyltransferase]; HIF1A [hypoxia inducible factor 1, alpha subunit (basic helix-loop-helix transcription factor)]; HINFP [histone H4 transcription factor]; HINT1 [histidine triad nucleotide binding protein 1]; HIPK2 [homeodomain interacting protein kinase 2]; HIRA [HIR histone cell cycle regulation defective homolog A (S. cerevisiae)]; HIST1H1B [histone cluster 1, H1b]; HIST1H3E [histone cluster 1, H3e]; H1ST2H2AC [histone cluster 2, H2ac]; HIST2H3C [histone cluster 2, H3c]; HIST4H4 [histone cluster 4, H4]; HJURP [Holliday junction recognition protein]; HK2 [hexokinase 2]; HLA-A [major histocompatibility complex, class I, A]; HLA-B [major histocompatibility complex, class I, B]; HLA-C [major histocompatibility complex, class I, C]; HLA-DMA [major histocompatibility complex, class II, DM alpha]; HLA-DMB [major histocompatibility complex, class II, DM beta]; HLA-DOA [major histocompatibility complex, class II, DO alpha]; HLA-DOB [major histocompatibility complex, class II, DO beta]; HLA-DPA1 [major histocompatibility complex, class II, DP alpha 1]; HLA-DPB1 [major histocompatibility complex, class II, DP beta 1]; HLA-DQA1 [major histocompatibility complex, class II, DQ alpha 1]; HLA-DQA2 [major histocompatibility complex, class II, DQ alpha 2]; HLA-DQB1 [major histocompatibility complex, class II, DQ beta 1]; HLA-DRA [major histocompatibility complex, class II, DR alpha]; HLA-DRB1 [major histocompatibility complex, class II, DR beta 1]; HLA-DRB3 [major histocompatibility complex, class II, DR beta 3]; HLA-DRB4 [major histocompatibility complex, class II, DR beta 4]; HLA-DRB5 [major histocompatibility complex, class II, DR beta 5]; HLA-E [major histocompatibility complex, class I, E]; HLA-F [major histocompatibility complex, class I, F]; HLA-G [major histocompatibility complex, class I, G]; HLCS [holocarboxylase synthetase (biotin-(proprionyl-Coenzyme A-carboxylase (ATP-hydrolysing)) ligase)]; HLTF [helicase-like transcription factor]; HLX [H2.0-like homeobox]; HMBS [hydroxymethylbilane synthase]; HMGA1 [high mobility group AT-hook 1]; HMGB1 [high-mobility group box 1]; HMGCR [3-hydroxy-3-methylglutaryl-Coenzyme A reductase]; HMOX1 [heme oxygenase (decycling) 1]; HMOX2 [heme oxygenase (decycling) 2]; HNF1A [HNF1 homeobox A]; HNF4A [hepatocyte nuclear factor 4, alpha]; HNMT [histamine N-methyltransferase]; HNRNPA1 [heterogeneous nuclear ribonucleoprotein A1]; HNRNPA2B1 [heterogeneous nuclear ribonucleoprotein A2/B1]; HNRNPH2 [heterogeneous nuclear ribonucleoprotein H2 (H′)]; HNRNPUL1 [heterogeneous nuclear ribonucleoprotein U-like 1]; HOXA13 [homeobox A13]; HOXA4 [homeobox A4]; HOXA9 [homeobox A9]; HOXB4 [homeobox B4]; HP [haptoglobin]; HPGDS [hematopoietic prostaglandin D synthase]; HPR [haptoglobin-related protein]; HPRT1 [hypoxanthine phosphoribosyltransferase 1]; HPS1 [Hermansky-Pudlak syndrome 1]; HPS3 [Hermansky-Pudlak syndrome 3]; HPS4 [Hermansky-Pudlak syndrome 4]; HPSE [heparanase]; HPX [hemopexin]; HRAS [v-Ha-ras Harvey rat sarcoma viral oncogene homolog]; HRG [histidine-rich glycoprotein]; HRH1 [histamine receptor H1]; HRH2 [histamine receptor H2]; HRH3 [histamine receptor H3]; HRH4 [histamine receptor H4]; HSD11B1 [hydroxysteroid (11-beta) dehydrogenase 1]; HSD11B2 [hydroxysteroid (11-beta) dehydrogenase 2]; HSD17B1 [hydroxysteroid (17-beta) dehydrogenase 1]; HSD17B4 [hydroxysteroid (17-beta) dehydrogenase 4]; HSF1 [heat shock transcription factor 1]; HSP90AA1 [heat shock protein 90 kDa alpha (cytosolic), class A member 1]; HSP90AB1 [heat shock protein 90 kDa alpha (cytosolic), class B member 1]; HSP90B1 [heat shock protein 90 kDa beta (Grp94), member 1]; HSPA14 [heat shock 70 kDa protein 14]; HSPA1A [heat shock 70 kDa protein 1A]; HSPA1B [heat shock 70 kDa protein 1B]; HSPA2 [heat shock 70 kDa protein 2]; HSPA4 [heat shock 70 kDa protein 4]; HSPA5 [heat shock 70 kDa protein 5 (glucose-regulated protein, 78 kDa)]; HSPA8 [heat shock 70 kDa protein 8]; HSPB1 [heat shock 27 kDa protein 1]; HSPB2 [heat shock 27 kDa protein 2]; HSPD1 [heat shock 60 kDa protein 1 (chaperonin)]; HSPE1 [heat shock 10 kDa protein 1 (chaperonin 10)]; HSPG2 [heparan sulfate proteoglycan 2]; HTN3 [histatin 3]; HTR1A [5-hydroxytryptamine (serotonin) receptor 1A]; HTR2A [5-hydroxytryptamine (serotonin) receptor 2A]; HTR3A [5-hydroxytryptamine (serotonin) receptor 3A]; HTRA1 [HtrA serine peptidase 1]; HTT [huntingtin]; HUS1 [HUS1 checkpoint homolog (S. pombe)]; HUWE1 [HECT, UBA and WWE domain containing 1]; HYAL1 [hyaluronoglucosaminidase 1]; HYLS1 [hydrolethalus syndrome 1]; IAPP [islet amyloid polypeptide]; IBSP [integrin-binding sialoprotein]; ICAM1 [intercellular adhesion molecule 1]; ICAM2 [intercellular adhesion molecule 2]; ICAM3 [intercellular adhesion molecule 3]; ICAM4 [intercellular adhesion molecule 4 (Landsteiner-Wiener blood group)]; ICOS [inducible T-cell co-stimulator]; ICOSLG [inducible T-cell co-stimulator ligand]; ID1 [inhibitor of DNA binding 1, dominant negative helix-loop-helix protein]; ID2 [inhibitor of DNA binding 2, dominant negative helix-loop-helix protein]; IDO1 [indoleamine 2 [3-dioxygenase 1]; IDS [iduronate 2-sulfatase]; IDUA [iduronidase, alpha-L-]; IF127 [interferon, alpha-inducible protein 27]; IFI30 [interferon, gamma-inducible protein 30]; IFITM1 [interferon induced transmembrane protein 1 (9-27)]; IFNA 1 [interferon, alpha 1]; IFNA 2 [interferon, alpha 2]; IFNAR1 [interferon (alpha, beta and omega) receptor 1]; IFNAR2 [interferon (alpha, beta and omega) receptor 2]; IFNB1 [interferon, beta 1, fibroblast]; IFNG [interferon, gamma]; IFNGR1 [interferon gamma receptor 1]; IFNGR2 [interferon gamma receptor 2 (interferon gamma transducer 1)]; IGF1 [insulin-like growth factor 1 (somatomedin C)]; IGF1R [insulin-like growth factor 1 receptor]; IGF2 [insulin-like growth factor 2 (somatomedin A)]; IGF2R [insulin-like growth factor 2 receptor]; IGFBP1 [insulin-like growth factor binding protein 1]; IGFBP2 [insulin-like growth factor binding protein 2, 36 kDa]; IGFBP3 [insulin-like growth factor binding protein 3]; IGFBP4 [insulin-like growth factor binding protein 4]; IGFBP5 [insulin-like growth factor binding protein 5]; IGHA1 [immunoglobulin heavy constant alpha 1]; IGHE [immunoglobulin heavy constant epsilon]; IGHG1 [immunoglobulin heavy constant gamma 1 (G1 m marker)]; IGHG3 [immunoglobulin heavy constant gamma 3 (G3m marker)]; IGHG4 [immunoglobulin heavy constant gamma 4 (G4m marker)]; IGHM [immunoglobulin heavy constant mu]; IGHMBP2 [immunoglobulin mu binding protein 2]; IGKC [immunoglobulin kappa constant]; IGKV2D-29 [immunoglobulin kappa variable 2D-29]; IGLL1 [immunoglobulin lambda-like polypeptide 1]; IGSF1 [immunoglobulin superfamily, member 1]; IKBKAP [inhibitor of kappa light polypeptide gene enhancer in B-cells, kinase complex-associated protein]; IKBKB [inhibitor of kappa light polypeptide gene enhancer in B-cells, kinase beta]; IKBKE [inhibitor of kappa light polypeptide gene enhancer in B-cells, kinase epsilon]; IKBKG [inhibitor of kappa light polypeptide gene enhancer in B-cells, kinase gamma]; IKZF1 [IKAROS family zinc finger 1 (Ikaros)]; IKZF2 [IKAROS family zinc finger 2 (Helios)]; IL10 [interleukin 10]; IL10RA [interleukin 10 receptor, alpha]; IL10RB [interleukin 10 receptor, beta]; IL11 [interleukin 11]; IL12A [interleukin 12A (natural killer cell stimulatory factor 1, cytotoxic lymphocyte maturation factor 1, p35)]; IL12B [interleukin 12B (natural killer cell stimulatory factor 2, cytotoxic lymphocyte maturation factor 2, p40)]; IL12RB1 [interleukin 12 receptor, beta 1]; IL12RB2 [interleukin 12 receptor, beta 2]; IL13 [interleukin 13]; IL13RA1 [interleukin 13 receptor, alpha 1]; IL13RA2 [interleukin 13 receptor, alpha 2]; IL15 [interleukin 15]; IL15RA [interleukin 15 receptor, alpha]; IL16 [interleukin 16 (lymphocyte chemoattractant factor)]; IL17A [interleukin 17A]; IL17F [interleukin 17F]; IL17RA [interleukin 17 receptor A]; IL17RB [interleukin 17 receptor B]; IL17RC [interleukin 17 receptor C]; IL18 [interleukin 18 (interferon-gamma-inducing factor)]; IL18BP [interleukin 18 binding protein]; IL18R1 [interleukin 18 receptor 1]; IL18RAP [interleukin 18 receptor accessory protein]; IL19 [interleukin 19]; ILIA [interleukin 1, alpha]; IL1B [interleukin 1, beta]; IL1F9 [interleukin 1 family, member 9]; IL1R1 [interleukin 1 receptor, type I]; IL1RAP [interleukin 1 receptor accessory protein]; IL1RL1 [interleukin 1 receptor-like 1]; IL1RN [interleukin 1 receptor antagonist]; IL2 [interleukin 2]; IL20 [interleukin 20]; IL21 [interleukin 21]; IL21R [interleukin 21 receptor]; IL22 [interleukin 22]; IL23A [interleukin 23, alpha subunit p19]; IL23R [interleukin 23 receptor]; IL24 [interleukin 24]; IL25 [interleukin 25]; IL26 [interleukin 26]; IL27 [interleukin 27]; IL27RA [interleukin 27 receptor, alpha]; IL29 [interleukin 29 (interferon, lambda 1)]; IL2RA [interleukin 2 receptor, alpha]; IL2RB [interleukin 2 receptor, beta]; IL2RG [interleukin 2 receptor, gamma (severe combined immunodeficiency)]; IL3 [interleukin 3 (colony-stimulating factor, multiple)]; IL31 [interleukin 31]; IL32 [interleukin 32]; IL33 [interleukin 33]; IL3RA [interleukin 3 receptor, alpha (low affinity)]; IL4 [interleukin 4]; IL4R [interleukin 4 receptor]; IL5 [interleukin 5 (colony-stimulating factor, eosinophil)]; IL5RA [interleukin 5 receptor, alpha]; IL6 [interleukin 6 (interferon, beta 2)]; IL6R [interleukin 6 receptor]; IL6ST [interleukin 6 signal transducer (gp130, oncostatin M receptor)]; IL7 [interleukin 7]; IL7R [interleukin 7 receptor]; IL8 [interleukin 8]; IL9 [interleukin 9]; IL9R [interleukin 9 receptor]; ILK [integrin-linked kinase]; IMP5 [intramembrane protease 5]; INCENP [inner centromere protein antigens 135/155 kDa]; ING1 [inhibitor of growth family, member 1]; INHA [inhibin, alpha]; INHBA [inhibin, beta A]; INPP4A [inositol polyphosphate-4-phosphatase, type I, 107 kDa]; INPP5D [inositol polyphosphate-5-phosphatase, 145 kDa]; INPP5E [inositol polyphosphate-5-phosphatase, 72 kDa]; INPPL1 [inositol polyphosphate phosphatase-like 1]; INS [insulin]; INSL3 [insulin-like 3 (Leydig cell)]; INSR [insulin receptor]; IPO13 [importin 13]; IPO7 [importin 7]; IQGAP1 [IQ motif containing GTPase activating protein 1]; IRAK1 [interleukin-1 receptor-associated kinase 1]; IRAK3 [interleukin-1 receptor-associated kinase 3]; IRAK4 [interleukin-1 receptor-associated kinase 4]; IRF1 [interferon regulatory factor 1]; IRF2 [interferon regulatory factor 2]; IRF3 [interferon regulatory factor 3]; IRF4 [interferon regulatory factor 4]; IRF5 [interferon regulatory factor 5]; IRF7 [interferon regulatory factor 7]; IRF8 [interferon regulatory factor 8]; IRGM [immunity-related GTPase family, M]; IRS1 [insulin receptor substrate 1]; IRS2 [insulin receptor substrate 2]; IRS4 [insulin receptor substrate 4]; ISG15 [ISG15 ubiquitin-like modifier]; ITCH [itchy E3 ubiquitin protein ligase homolog (mouse)]; ITFG1 [integrin alpha FG-GAP repeat containing 1]; ITGA1 [integrin, alpha 1]; ITGA2 [integrin, alpha 2 (CD49B, alpha 2 subunit of VLA-2 receptor)]; ITGA2B [integrin, alpha 2b (platelet glycoprotein IIb of IIb/IIIa complex, antigen CD41)]; ITGA3 [integrin, alpha 3 (antigen CD49C, alpha 3 subunit of VLA-3 receptor)]; ITGA4 [integrin, alpha 4 (antigen CD49D, alpha 4 subunit of VLA-4 receptor)]; ITGA5 [integrin, alpha 5 (fibronectin receptor, alpha polypeptide)]; ITGA6 [integrin, alpha 6]; ITGA8 [integrin, alpha 8]; ITGAE [integrin, alpha E (antigen CD103, human mucosal lymphocyte antigen 1; alpha polypeptide)]; ITGAL [integrin, alpha L (antigen CD11A (p180), lymphocyte function-associated antigen 1; alpha polypeptide)]; ITGAM [integrin, alpha M (complement component 3 receptor 3 subunit)]; ITGAV [integrin, alpha V (vitronectin receptor, alpha polypeptide, antigen CD51)]; ITGAX [integrin, alpha X (complement component 3 receptor 4 subunit)]; ITGB1 [integrin, beta 1 (fibronectin receptor, beta polypeptide, antigen CD29 includes MDF2, MSK12)]; ITGB2 [integrin, beta 2 (complement component 3 receptor 3 and 4 subunit)]; ITGB3 [integrin, beta 3 (platelet glycoprotein IIIa, antigen CD61)]; ITGB3BP [integrin beta 3 binding protein (beta3-endonexin)]; ITGB4 [integrin, beta 4]; ITGB6 [integrin, beta 6]; ITGB7 [integrin, beta 7]; ITIH4 [inter-alpha (globulin) inhibitor H4 (plasma Kallikrein-sensitive glycoprotein)]; ITK [IL2-inducible T-cell kinase]; ITLN1 [intelectin 1 (galactofuranose binding)]; ITLN2 [intelectin 2]; ITPA [inosine triphosphatase (nucleoside triphosphate pyrophosphatase)]; ITPR1 [inositol 1,4,5-triphosphate receptor, type 1]; ITPR3 [inositol 1,4,5-triphosphate receptor, type 3]; IVD [isovaleryl Coenzyme A dehydrogenase]; IVL [involucrin]; IVNS1ABP [influenza virus NS1A binding protein]; JAG1 [jagged 1 (Alagille syndrome)]; JAK1 [Janus kinase 1]; JAK2 [Janus kinase 2]; JAK3 [Janus kinase 3]; JAKMIP1 [janus kinase and microtubule interacting protein 1]; JMJD6 [jumonji domain containing 6]; JPH4 [junctophilin 4]; JRKL [jerky homolog-like (mouse)]; JUN [jun oncogene]; JUND [jun D proto-oncogene]; JUP [junction plakoglobin]; KARS [lysyl-tRNA synthetase]; KAT5 [K(lysine) acetyltransferase 5]; KCNA2 [potassium voltage-gated channel, shaker-related subfamily, member 2]; KCNA5 [potassium voltage-gated channel, shaker-related subfamily, member 5]; KCND1 [potassium voltage-gated channel, Shal-related subfamily, member 1]; KCNH2 [potassium voltage-gated channel, subfamily H (eag-related), member 2]; KCNIP4 [Kv channel interacting protein 4]; KCNMA1 [potassium large conductance calcium-activated channel, subfamily M, alpha member 1]; KCNMB1 [potassium large conductance calcium-activated channel, subfamily M, beta member 1]; KCNN3 [potassium intermediate/small conductance calcium-activated channel, subfamily N, member 3]; KCNS3 [potassium voltage-gated channel, delayed-rectifier, subfamily S, member 3]; KDR [kinase insert domain receptor (a type III receptor tyrosine kinase)]; KHDRBS1 [KH domain containing, RNA binding, signal transduction associated 1]; KHDRBS3 [KH domain containing, RNA binding, signal transduction associated 3]; KIAA0101 [KIAA0101]; KIF16B [kinesin family member 16B]; KIF20B [kinesin family member 20B]; KIF21B [kinesin family member 21B]; KIF22 [kinesin family member 22]; KIF2B [kinesin family member 2B]; KIF2C [kinesin family member 20]; KIR2DL1 [killer cell immunoglobulin-like receptor, two domains, long cytoplasmic tail, 1]; KIR2DL2 [killer cell immunoglobulin-like receptor, two domains, long cytoplasmic tail, 2]; KIR2DL3 [killer cell immunoglobulin-like receptor, two domains, long cytoplasmic tail, 3]; KIR2DL5A [killer cell immunoglobulin-like receptor, two domains, long cytoplasmic tail, 5A]; KIR2DS1 [killer cell immunoglobulin-like receptor, two domains, short cytoplasmic tail, 1]; KIR2DS2 [killer cell immunoglobulin-like receptor, two domains, short cytoplasmic tail, 2]; KIR2DS5 [killer cell immunoglobulin-like receptor, two domains, short cytoplasmic tail, 5]; KIR3DL1 [killer cell immunoglobulin-like receptor, three domains, long cytoplasmic tail, 1]; KIR3DS1 [killer cell immunoglobulin-like receptor, three domains, short cytoplasmic tail, 1]; KISS1 [KiSS-1 metastasis-suppressor]; KISS1R [KISS1 receptor]; KIT [v-kit Hardy-Zuckerman 4 feline sarcoma viral oncogene homolog]; KITLG [KIT ligand]; KLF2 [Kruppel-like factor 2 (lung)]; KLF4 [Kruppel-like factor 4 (gut)]; KLK1 [kallikrein 1]; KLK11 [kallikrein-related peptidase 11]; KLK3 [kallikrein-related peptidase 3]; KLKB1 [kallikrein B, plasma (Fletcher factor) 1]; KLRB1 [killer cell lectin-like receptor subfamily B, member 1]; KLRC1 [killer cell lectin-like receptor subfamily C, member 1]; KLRD1 [killer cell lectin-like receptor subfamily D, member 1]; KLRK1 [killer cell lectin-like receptor subfamily K, member 1]; KNG1 [kininogen 1]; KPNA1 [karyopherin alpha 1 (importin alpha 5)]; KPNA2 [karyopherin alpha 2 (RAG cohort 1, importin alpha 1)]; KPNB1 [karyopherin (importin) beta 1]; KRAS [v-Ki-ras2 Kirsten rat sarcoma viral oncogene homolog]; KRT1 [keratin 1]; KRT10 [keratin 10]; KRT13 [keratin 13]; KRT14 [keratin 14]; KRT16 [keratin 16]; KRT18 [keratin 18]; KRT19 [keratin 19]; KRT20 [keratin 20]; KRT5 [keratin 5]; KRT7 [keratin 7]; KRT8 [keratin 8]; KRT9 [keratin 9]; KRTAP19-3 [keratin associated protein 19-3]; KRTAP2-1, keratin associated protein 2-1]; L1CAM [L1 cell adhesion molecule]; LACTB [lactamase, beta]; LAG3 [lymphocyte-activation gene 3]; LALBA [lactalbumin, alpha-]; LAMA1 [laminin, alpha 1]; LAMA2 [laminin, alpha 2]; LAMA3 [laminin, alpha 3]; LAMA4 [laminin, alpha 4]; LAMB1 [laminin, beta 1]; LAMB2 [laminin, beta 2 (laminin S)]; LAMB3 [laminin, beta 3]; LAMC1 [laminin, gamma 1 (formerly LAMB2)]; LAMC2 [laminin, gamma 2]; LAMP1 [lysosomal-associated membrane protein 1]; LAMP2 [lysosomal-associated membrane protein 2]; LAMP3 [lysosomal-associated membrane protein 3]; LAP3 [leucine aminopeptidase 3]; LAPTM4A [lysosomal protein transmembrane 4 alpha]; LAT [linker for activation of T cells]; LBP [lipopolysaccharide binding protein]; LBR [lamin B receptor]; LBXCOR1 [Lbxcor1 homolog (mouse)]; LCAT [lecithin-cholesterol acyltransferase]; LCK [lymphocyte-specific protein tyrosine kinase]; LCN1 [lipocalin 1 (tear prealbumin)]; LCN2 [lipocalin 2]; LCP1 [lymphocyte cytosolic protein 1 (L-plastin)]; LCT [lactase]; LDLR [low density lipoprotein receptor]; LDLRAP1 [low density lipoprotein receptor adaptor protein 1]; LECT2 [leukocyte cell-derived chemotaxin 2]; LELP1 [late cornified envelope-like proline-rich 1]; LEMD3 [LEM domain containing 3]; LEP [leptin]; LEPR [leptin receptor]; LGALS1 [lectin, galactoside-binding, soluble, 1]; LGALS3 [lectin, galactoside-binding, soluble, 3]; LGALS3BP [lectin, galactoside-binding, soluble, 3 binding protein]; LGALS4 [lectin, galactoside-binding, soluble, 4]; LGALS9 [lectin, galactoside-binding, soluble, 9]; LGALS9B [lectin, galactoside-binding, soluble, 9B]; LGR4 [leucine-rich repeat-containing G protein-coupled receptor 4]; LHCGR [luteinizing hormone/choriogonadotropin receptor]; LIF [leukemia inhibitory factor (cholinergic differentiation factor)]; LIFR [leukemia inhibitory factor receptor alpha]; LIG1 [ligase I, DNA, ATP-dependent]; LIG3 [ligase III, DNA, ATP-dependent]; LIG4 [ligase IV, DNA, ATP-dependent]; LILRA3 [leukocyte immunoglobulin-like receptor, subfamily A (without TM domain), member 3]; LILRB4 [leukocyte immunoglobulin-like receptor, subfamily B (with TM and ITIM domains), member 4]; LIMS1 [LIM and senescent cell antigen-like domains 1]; LIPA [lipase A, lysosomal acid, cholesterol esterase]; LIPC [lipase, hepatic]; LIPE [lipase, hormone-sensitive]; LIPG [lipase, endothelial]; LMAN1 [lectin, mannose-binding, 1]; LMLN [leishmanolysin-like (metallopeptidase M8 family)]; LMNA [lamin NC]; LMNB1 [lamin B1]; LMNB2 [lamin B2]; LOC646627 [phospholipase inhibitor]; LOX [lysyl oxidase]; LOXHD1 [lipoxygenase homology domains 1]; LOXL1 [lysyl oxidase-like 1]; LPA [lipoprotein, Lp(a)]; LPAR3 [lysophosphatidic acid receptor 3]; LPCAT2 [lysophosphatidylcholine acyltransferase 2]; LPL [lipoprotein lipase]; LPO [lactoperoxidase]; LPP [LIM domain containing preferred translocation partner in lipoma]; LRBA [LPS-responsive vesicle trafficking, beach and anchor containing]; LRP1 [low density lipoprotein receptor-related protein 1]; LRP6 [low density lipoprotein receptor-related protein 6]; LRPAP1 [low density lipoprotein receptor-related protein associated protein 1]; LRRC32 [leucine rich repeat containing 32]; LRRC37B [leucine rich repeat containing 37B]; LRRC8A [leucine rich repeat containing 8 family, member A]; LRRK2 [leucine-rich repeat kinase 2]; LRTOMT [leucine rich transmembrane and O-methyltransferase domain containing]; LSM1 [LSM1 homolog, U6 small nuclear RNA associated (S. cerevisiae)]; LSM2 [LSM2 homolog, U6 small nuclear RNA associated (S. cerevisiae)]; LSP1 [lymphocyte-specific protein 1]; LTA [lymphotoxin alpha (TNF superfamily, member 1)]; LTA4H [leukotriene A4 hydrolase]; LTB [lymphotoxin beta (TNF superfamily, member 3)]; LTB4R [leukotriene B4 receptor]; LTB4R2 [leukotriene B4 receptor 2]; LTBR [lymphotoxin beta receptor (TNFR superfamily, member 3)]; LTC4S [leukotriene C4 synthase]; LTF [lactotransferrin]; LY86 [lymphocyte antigen 86]; LY9 [lymphocyte antigen 9]; LYN [v-yes-1 Yamaguchi sarcoma viral related oncogene homolog]; LYRM4 [LYR motif containing 4]; LYST [lysosomal trafficking regulator]; LYZ [lysozyme (renal amyloidosis)]; LYZL6 [lysozyme-like 6]; LZTR1 [leucine-zipper-like transcription regulator 1]; M6PR [mannose-6-phosphate receptor (cation dependent)]; MADCAM1 [mucosal vascular addressin cell adhesion molecule 1]; MAF [v-maf musculoaponeurotic fibrosarcoma oncogene homolog (avian)]; MAG [myelin associated glycoprotein]; MAN2A1 [mannosidase, alpha, class 2A, member 1]; MAN2B1 [mannosidase, alpha, class 2B, member 1]; MANBA [mannosidase, beta A, lysosomal]; MANF [mesencephalic astrocyte-derived neurotrophic factor]; MAOB [monoamine oxidase B]; MAP2 [microtubule-associated protein 2]; MAP2K1 [mitogen-activated protein kinase kinase 1]; MAP2K2 [mitogen-activated protein kinase kinase 2]; MAP2K3 [mitogen-activated protein kinase kinase 3]; MAP2K4 [mitogen-activated protein kinase kinase 4]; MAP3K1 [mitogen-activated protein kinase kinase kinase 1]; MAP3K11 [mitogen-activated protein kinase kinase kinase 11]; MAP3K14 [mitogen-activated protein kinase kinase kinase 14]; MAP3K5 [mitogen-activated protein kinase kinase kinase 5]; MAP3K7 [mitogen-activated protein kinase kinase kinase 7]; MAP3K9 [mitogen-activated protein kinase kinase kinase 9]; MAPK1 [mitogen-activated protein kinase 1]; MAPK10 [mitogen-activated protein kinase 10]; MAPK11 [mitogen-activated protein kinase 11]; MAPK12 [mitogen-activated protein kinase 12]; MAPK13 [mitogen-activated protein kinase 13]; MAPK14 [mitogen-activated protein kinase 14]; MAPK3 [mitogen-activated protein kinase 3]; MAPK8 [mitogen-activated protein kinase 8]; MAPK9 [mitogen-activated protein kinase 9]; MAPKAP1 [mitogen-activated protein kinase associated protein 1]; MAPKAPK2 [mitogen-activated protein kinase-activated protein kinase 2]; MAPKAPK5 [mitogen-activated protein kinase-activated protein kinase 5]; MAPT [microtubule-associated protein tau]; MARCKS [myristoylated alanine-rich protein kinase C substrate]; MASP2 [mannan-binding lectin serine peptidase 2]; MATN1 [matrilin 1, cartilage matrix protein]; MAVS [mitochondrial antiviral signaling protein]; MB [myoglobin]; MBD2 [methyl-CpG binding domain protein 2]; MBL2 [mannose-binding lectin (protein C) 2, soluble (opsonic defect)]; MBP [myelin basic protein]; MBTPS2 [membrane-bound transcription factor peptidase, site 2]; MC2R [melanocortin 2 receptor (adrenocorticotropic hormone)]; MC3R [melanocortin 3 receptor]; MC4R [melanocortin 4 receptor]; MCCC2 [methylcrotonoyl-Coenzyme A carboxylase 2 (beta)]; MCHR1 [melanin-concentrating hormone receptor 1]; MCL1 [myeloid cell leukemia sequence 1 (BCL2-related)]; MCM2 [minichromosome maintenance complex component 2]; MCM4 [minichromosome maintenance complex component 4]; MCOLN1 [mucolipin 1]; MCPH1 [microcephalin 1]; MDC1 [mediator of DNA-damage checkpoint 1]; MDH2 [malate dehydrogenase 2, NAD (mitochondrial)]; MDM2 [Mdm2 p53 binding protein homolog (mouse)]; ME2 [malic enzyme 2, NAD(+)-dependent, mitochondrial]; MECOM [MDS1 and EVI1 complex locus]; MED1 [mediator complex subunit 1]; MED12 [mediator complex subunit 12]; MED15 [mediator complex subunit 15]; MED28 [mediator complex subunit 28]; MEFV [Mediterranean fever]; MEN1 [multiple endocrine neoplasia I]; MEPE [matrix extracellular phosphoglycoprotein]; MERTK [c-mer proto-oncogene tyrosine kinase]; MESP2 [mesoderm posterior 2 homolog (mouse)]; MET [met proto-oncogene (hepatocyte growth factor receptor)]; MGAM [maltase-glucoamylase (alpha-glucosidase)]; MGAT1 [mannosyl (alpha-1,3-)-glycoprotein beta-1,2-N-acetylglucosaminyltransferase]; MGAT2 [mannosyl (alpha-1,6-)-glycoprotein beta-1,2-N-acetylglucosaminyltransferase]; MGLL [monoglyceride lipase]; MGMT [O-6-methylguanine-DNA methyltransferase]; MGST2 [microsomal glutathione S-transferase 2]; MICA [MHC class I polypeptide-related sequence A]; MICB [MHC class I polypeptide-related sequence B]; MIF [macrophage migration inhibitory factor (glycosylation-inhibiting factor)]; MKI67 [antigen identified by monoclonal antibody Ki-67]; MKS1 [Meckel syndrome, type 1]; MLH1 [mutL homolog 1, colon cancer, nonpolyposis type 2 (E. coli)]; MLL [myeloid/lymphoid or mixed-lineage leukemia (trithorax homolog, Drosophila)]; MLLT4 [myeloid/lymphoid or mixed-lineage leukemia (trithorax homolog, Drosophila); translocated to, 4]; MLN [motilin]; MLXIPL [MLX interacting protein-like]; MMAA [methylmalonic aciduria (cobalamin deficiency) cblA type]; MMAB [methylmalonic aciduria (cobalamin deficiency) cblB type]; MMACHC [methylmalonic aciduria (cobalamin deficiency) cblC type, with homocystinuria]; MME [membrane metallo-endopeptidase]; MMP1 [matrix metallopeptidase 1 (interstitial collagenase)]; MMP10 [matrix metallopeptidase 10 (stromelysin 2)]; MMP12 [matrix metallopeptidase 12 (macrophage elastase)]; MMP13 [matrix metallopeptidase 13 (collagenase 3)]; MMP14 [matrix metallopeptidase 14 (membrane-inserted)]; MMP15 [matrix metallopeptidase 15 (membrane-inserted)]; MMP17 [matrix metallopeptidase 17 (membrane-inserted)]; MMP2 [matrix metallopeptidase 2 (gelatinase A, 72 kDa gelatinase, 72 kDa type IV collagenase)]; MMP20 [matrix metallopeptidase 20]; MMP21 [matrix metallopeptidase 21]; MMP28 [matrix metallopeptidase 28]; MMP3 [matrix metallopeptidase 3 (stromelysin 1, progelatinase)]; MMP7 [matrix metallopeptidase 7 (matrilysin, uterine)]; MMP8 [matrix metallopeptidase 8 (neutrophil collagenase)]; MMP9 [matrix metallopeptidase 9 (gelatinase B, 92 kDa gelatinase, 92 kDa type IV collagenase)]; MMRN1 [multimerin 1]; MNAT1 [menage a trois homolog 1, cyclin H assembly factor (Xenopus laevis)]; MOG [myelin oligodendrocyte glycoprotein]; MOGS [mannosyl-oligosaccharide glucosidase]; MPG [N-methylpurine-DNA glycosylase]; MPL [myeloproliferative leukemia virus oncogene]; MPO [myeloperoxidase]; MPZ [myelin protein zero]; MR1 [major histocompatibility complex, class I-related]; MRC1 [mannose receptor, C type 1]; MRC2 [mannose receptor, C type 2]; MRE11A [MRE11 meiotic recombination 11 homolog A (S. cerevisiae)]; MRGPRX1 [MAS-related GPR, member X1]; MRPL28 [mitochondrial ribosomal protein L28]; MRPL40 [mitochondrial ribosomal protein L40]; MRPS16 [mitochondrial ribosomal protein S16]; MRPS22 [mitochondrial ribosomal protein S22]; MS4A1 [membrane-spanning 4-domains, subfamily A, member 1]; MS4A2 [membrane-spanning 4-domains, subfamily A, member 2 (Fc fragment of IgE, high affinity I, receptor for; beta polypeptide)]; MS4A3 [membrane-spanning 4-domains, subfamily A, member 3 (hematopoietic cell-specific)]; MSH2 [mutS homolog 2, colon cancer, nonpolyposis type 1 (E. coli)]; MSH5 [mutS homolog 5 (E. coli)]; MSH6 [mutS homolog 6 (E. coli)]; MSLN [mesothelin]; MSN [moesin]; MSR1 [macrophage scavenger receptor 1]; MST1 [macrophage stimulating 1 (hepatocyte growth factor-like)]; MST1R [macrophage stimulating 1 receptor (c-met-related tyrosine kinase)]; MSTN [myostatin]; MSX2 [msh homeobox 2]; MT2A [metallothionein 2A]; MTCH2 [mitochondrial carrier homolog 2 (C. elegans)]; MT-CO2 [mitochondrially encoded cytochrome c oxidase II]; MTCP1 [mature T-cell proliferation 1]; MT-CYB [mitochondrially encoded cytochrome b]; MTHFD1 [methylenetetrahydrofolate dehydrogenase (NADP+ dependent) 1, methenyltetrahydrofolate cyclohydrolase, formyltetrahydrofolate synthetase]; MTHFR [5 [10-methylenetetrahydrofolate reductase (NADPH)]; MTMR14 [myotubularin related protein 14]; MTMR2 [myotubularin related protein 2]; MT-ND1 [mitochondrially encoded NADH dehydrogenase 1]; MT-ND2 [mitochondrially encoded NADH dehydrogenase 2]; MTOR [mechanistic target of rapamycin (serine/threonine kinase)]; MTR [5-methyltetrahydrofolate-homocysteine methyltransferase]; MTRR [5-methyltetrahydrofolate-homocysteine methyltransferase reductase]; MTTP [microsomal triglyceride transfer protein]; MTX1 [metaxin 1]; MUC1 [mucin 1, cell surface associated]; MUC12 [mucin 12, cell surface associated]; MUC16 [mucin 16, cell surface associated]; MUC19 [mucin 19, oligomeric]; MUC2 [mucin 2, oligomeric mucus/gel-forming]; MUC3A [mucin 3A, cell surface associated]; MUC3B [mucin 3B, cell surface associated]; MUC4 [mucin 4, cell surface associated]; MUC5AC [mucin SAC, oligomeric mucus/gel-forming]; MUC5B [mucin 5B, oligomeric mucus/gel-forming]; MUC6 [mucin 6, oligomeric mucus/gel-forming]; MUC7 [mucin 7, secreted]; MUS81 [MUS81 endonuclease homolog (S. cerevisiae)]; MUSK [muscle, skeletal, receptor tyrosine kinase]; MUT [methylmalonyl Coenzyme A mutase]; MVK [mevalonate kinase]; MVP [major vault protein]; MX1 [myxovirus (influenza virus) resistance 1, interferon-inducible protein p78 (mouse)]; MYB [v-myb myeloblastosis viral oncogene homolog (avian)]; MYBPH [myosin binding protein H]; MYC [v-myc myelocytomatosis viral oncogene homolog (avian)]; MYCN [v-myc myelocytomatosis viral related oncogene, neuroblastoma derived (avian)]; MYD88 [myeloid differentiation primary response gene (88)]; MYH1 [myosin, heavy chain 1, skeletal muscle, adult]; MYH10 [myosin, heavy chain 10, non-muscle]; MYH11 [myosin, heavy chain 11, smooth muscle]; MYH14 [myosin, heavy chain 14, non-muscle]; MYH2 [myosin, heavy chain 2, skeletal muscle, adult]; MYH3 [myosin, heavy chain 3, skeletal muscle, embryonic]; MYH6 [myosin, heavy chain 6, cardiac muscle, alpha]; MYH7 [myosin, heavy chain 7, cardiac muscle, beta]; MYH8 [myosin, heavy chain 8, skeletal muscle, perinatal]; MYH9 [myosin, heavy chain 9, non-muscle]; MYL2 [myosin, light chain 2, regulatory, cardiac, slow]; MYL3 [myosin, light chain 3, alkali; ventricular, skeletal, slow]; MYL7 [myosin, light chain 7, regulatory]; MYL9 [myosin, light chain 9, regulatory]; MYLK [myosin light chain kinase]; MYO15A [myosin XVA]; MYO1A [myosin IA]; MYO1F [myosin IF]; MYO3A [myosin IIIA]; MYO5A [myosin VA (heavy chain 12, myoxin)]; MYO6 [myosin VI]; MYO7A [myosin VIIA]; MYO9B [myosin IXB]; MYOC [myocilin, trabecular meshwork inducible glucocorticoid response]; MYOD1 [myogenic differentiation 1]; MYOM2 [myomesin (M-protein) 2, 165 kDa]; MYST1 [MYST histone acetyltransferase 1]; MYST2 [MYST histone acetyltransferase 2]; MYST3 [MYST histone acetyltransferase (monocytic leukemia) 3]; MYST4 [MYST histone acetyltransferase (monocytic leukemia) 4]; NAGA [N-acetylgalactosaminidase, alpha-]; NAGLU [N-acetylglucosaminidase, alpha-]; NAMPT [nicotinamide phosphoribosyltransferase]; NANOG [Nanog homeobox]; NANOS1 [nanos homolog 1 (Drosophila)]; NAPA [N-ethylmaleimide-sensitive factor attachment protein, alpha]; NAT1 [N-acetyltransferase 1 (arylamine N-acetyltransferase)]; NAT2 [N-acetyltransferase 2 (arylamine N-acetyltransferase)]; NAT9 [N-acetyltransferase 9 (GCN5-related, putative)]; NBEA [neurobeachin]; NBN [nibrin]; NCAM1 [neural cell adhesion molecule 1]; NCF1 [neutrophil cytosolic factor 1]; NCF2 [neutrophil cytosolic factor 2]; NCF4 [neutrophil cytosolic factor 4, 40 kDa]; NCK1 [NCK adaptor protein 1]; NCL [nucleolin]; NCOA1 [nuclear receptor coactivator 1]; NCOA2 [nuclear receptor coactivator 2]; NCOR1 [nuclear receptor co-repressor 1]; NCR3 [natural cytotoxicity triggering receptor 3]; NDUFA13 [NADH dehydrogenase (ubiquinone) 1 alpha subcomplex, 13]; NDUFAB1 [NADH dehydrogenase (ubiquinone) 1, alpha/beta subcomplex, 1, 8 kDa]; NDUFAF2 [NADH dehydrogenase (ubiquinone) 1 alpha subcomplex, assembly factor 2]; NEDD4 [neural precursor cell expressed, developmentally down-regulated 4]; NEFL [neurofilament, light polypeptide]; NEFM [neurofilament, medium polypeptide]; NEGR1 [neuronal growth regulator 1]; NEK6 [NIMA (never in mitosis gene a)-related kinase 6]; NELF [nasal embryonic LHRH factor]; NELL1 [NEL-like 1 (chicken)]; NES [nestin]; NEU1 [sialidase 1 (lysosomal sialidase)]; NEUROD1 [neurogenic differentiation 1]; NF1 [neurofibromin 1]; NF2 [neurofibromin 2 (merlin)]; NFAT5 [nuclear factor of activated T-cells 5, tonicity-responsive]; NFATC1 [nuclear factor of activated T-cells, cytoplasmic, calcineurin-dependent 1]; NFATC2 [nuclear factor of activated T-cells, cytoplasmic, calcineurin-dependent 2]; NFATC4 [nuclear factor of activated T-cells, cytoplasmic, calcineurin-dependent 4]; NFE2L2 [nuclear factor (erythroid-derived 2)-like 2]; NFKB1 [nuclear factor of kappa light polypeptide gene enhancer in B-cells 1]; NFKB2 [nuclear factor of kappa light polypeptide gene enhancer in B-cells 2 (p49/p100)]; NFKBIA [nuclear factor of kappa light polypeptide gene enhancer in B-cells inhibitor, alpha]; NFKBIB [nuclear factor of kappa light polypeptide gene enhancer in B-cells inhibitor, beta]; NFKBIL1 [nuclear factor of kappa light polypeptide gene enhancer in B-cells inhibitor-like 1]; NFU1 [NFU1 iron-sulfur cluster scaffold homolog (S. cerevisiae)]; NGF [nerve growth factor (beta polypeptide)]; NGFR [nerve growth factor receptor (TNFR superfamily, member 16)]; NHEJ1 [nonhomologous end-joining factor 1]; NID1 [nidogen 1]; NKAP [NFkB activating protein]; NKX2-1, NK2 homeobox 1]; NKX2-3 [NK2 transcription factor related, locus 3 (Drosophila)]; NLRP3 [NLR family, pyrin domain containing 3]; NMB [neuromedin B]; NME1 [non-metastatic cells 1, protein (NM23A) expressed in]; NME2 [non-metastatic cells 2, protein (NM23B) expressed in]; NMU [neuromedin U]; NNAT [neuronatin]; NOD1 [nucleotide-binding oligomerization domain containing 1]; NOD2 [nucleotide-binding oligomerization domain containing 2]; NONO [non-POU domain containing, octamer-binding]; NOS1 [nitric oxide synthase 1 (neuronal)]; NOS2 [nitric oxide synthase 2, inducible]; NOS3 [nitric oxide synthase 3 (endothelial cell)]; NOTCH1 [Notch homolog 1, translocation-associated (Drosophila)]; NOTCH2 [Notch homolog 2 (Drosophila)]; NOTCH3 [Notch homolog 3 (Drosophila)]; NOTCH4 [Notch homolog 4 (Drosophila)]; NOX1 [NADPH oxidase 1]; NOX3 [NADPH oxidase 3]; NOX4 [NADPH oxidase 4]; NOX5 [NADPH oxidase, EF-hand calcium binding domain 5]; NPAT [nuclear protein, ataxia-telangiectasia locus]; NPC1 [Niemann-Pick disease, type C1]; NPC1L1 [NPC1 (Niemann-Pick disease, type C1, gene)-like 1]; NPC2 [Niemann-Pick disease, type C2]; NPHP1 [nephronophthisis 1 (juvenile)]; NPHS1 [nephrosis 1, congenital, Finnish type (nephrin)]; NPHS2 [nephrosis 2, idiopathic, steroid-resistant (podocin)]; NPLOC4 [nuclear protein localization 4 homolog (S. cerevisiae)]; NPM1 [nucleophosmin (nucleolar phosphoprotein B23, numatrin)]; NPPA [natriuretic peptide precursor A]; NPPB [natriuretic peptide precursor B]; NPPC [natriuretic peptide precursor C]; NPR1 [natriuretic peptide receptor A/guanylate cyclase A (atrionatriuretic peptide receptor A)]; NPR3 [natriuretic peptide receptor C/guanylate cyclase C (atrionatriuretic peptide receptor C)]; NPS [neuropeptide S]; NPSR1 [neuropeptide S receptor 1]; NPY [neuropeptide Y]; NPY2R [neuropeptide Y receptor Y2]; NQO1 [NAD(P)H dehydrogenase, quinone 1]; NR0B1 [nuclear receptor subfamily 0, group B, member 1]; NR1H2 [nuclear receptor subfamily 1, group H, member 2]; NR1H3 [nuclear receptor subfamily 1, group H, member 3]; NR1H4 [nuclear receptor subfamily 1, group H, member 4]; NR1I2 [nuclear receptor subfamily 1, group I, member 2]; NR1I3 [nuclear receptor subfamily 1, group I, member 3]; NR2F2 [nuclear receptor subfamily 2, group F, member 2]; NR3C1 [nuclear receptor subfamily 3, group C, member 1 (glucocorticoid receptor)]; NR3C2 [nuclear receptor subfamily 3, group C, member 2]; NR4A1 [nuclear receptor subfamily 4, group A, member 1]; NR4A3 [nuclear receptor subfamily 4, group A, member 3]; NR5A1 [nuclear receptor subfamily 5, group A, member 1]; NRF1 [nuclear respiratory factor 1]; NRG1 [neuregulin 1]; NRIP1 [nuclear receptor interacting protein 1]; NRIP2 [nuclear receptor interacting protein 2]; NRP1 [neuropilin 1]; NSD1 [nuclear receptor binding SET domain protein 1]; NSDHL [NAD(P) dependent steroid dehydrogenase-like]; NSF [N-ethylmaleimide-sensitive factor]; NT5E [5′-nucleotidase, ecto (CD73)]; NTAN1 [N-terminal asparagine amidase]; NTF3 [neurotrophin 3]; NTF4 [neurotrophin 4]; NTN1 [netrin 1]; NTRK1 [neurotrophic tyrosine kinase, receptor, type 1]; NTRK2 [neurotrophic tyrosine kinase, receptor, type 2]; NTRK3 [neurotrophic tyrosine kinase, receptor, type 3]; NTS [neurotensin]; NUCB2 [nucleobindin 2]; NUDT1 [nudix (nucleoside diphosphate linked moiety X)-type motif 1]; NUDT2 [nudix (nucleoside diphosphate linked moiety X)-type motif 2]; NUDT6 [nudix (nucleoside diphosphate linked moiety X)-type motif 6]; NUFIP2 [nuclear fragile X mental retardation protein interacting protein 2]; NUP98 [nucleoporin 98 kDa]; NXF1 [nuclear RNA export factor 1]; OCA2 [oculocutaneous albinism II]; OCLN [occludin]; ODC1 [ornithine decarboxylase 1]; OFD1 [oral-facial-digital syndrome 1]; OGDH [oxoglutarate (alpha-ketoglutarate) dehydrogenase (lipoamide)]; OGG1 [8-oxoguanine DNA glycosylase]; OGT [O-linked N-acetylglucosamine (GlcNAc) transferase (UDP-N-acetylglucosamine:polypeptide-N-acetylglucosaminyl transferase)]; OLR1 [oxidized low density lipoprotein (lectin-like) receptor 1]; OMP [olfactory marker protein]; ONECUT2 [one cut homeobox 2]; OPN3 [opsin 3]; OPRK1 [opioid receptor, kappa 1]; OPRM1 [opioid receptor, mu 1]; OPTN [optineurin]; OR2B11 [olfactory receptor, family 2, subfamily B, member 11]; ORMDL3 [ORM1-like 3 (S. cerevisiae)]; OSBP [oxysterol binding protein]; OSGIN2 [oxidative stress induced growth inhibitor family member 2]; OSM [oncostatin M]; OTC [ornithine carbamoyltransferase]; OTOP2 [otopetrin 2]; OTOP3 [otopetrin 3]; OTUD1 [OTU domain containing 1]; OXA1L [oxidase (cytochrome c) assembly 1-like]; OXER1 [oxoeicosanoid (OXE) receptor 1]; OXT [oxytocin, prepropeptide]; OXTR [oxytocin receptor]; P2RX7 [purinergic receptor P2X, ligand-gated ion channel, 7]; P2RY1 [purinergic receptor P2Y, G-protein coupled, 1]; P2RY12 [purinergic receptor P2Y, G-protein coupled, 12]; P2RY14 [purinergic receptor P2Y, G-protein coupled, 14]; P2RY2 [purinergic receptor P2Y, G-protein coupled, 2]; P4HA2 [prolyl 4-hydroxylase, alpha polypeptide II]; P4HB [prolyl 4-hydroxylase, beta polypeptide]; P4HTM [prolyl 4-hydroxylase, transmembrane (endoplasmic reticulum)]; PABPC1 [poly(A) binding protein, cytoplasmic 1]; PACSIN3 [protein kinase C and casein kinase substrate in neurons 3]; PAEP [progestagen-associated endometrial protein]; PAFAH1B1 [platelet-activating factor acetylhydrolase 1b, regulatory subunit 1 (45 kDa)]; PAH [phenylalanine hydroxylase]; PAK1 [p21 protein (Cdc42/Rac)-activated kinase 1]; PAK2 [p21 protein (Cdc42/Rac)-activated kinase 2]; PAK3 [p21 protein (Cdc42/Rac)-activated kinase 3]; PAM [peptidylglycine alpha-amidating monooxygenase]; PAPPA [pregnancy-associated plasma protein A, pappalysin 1]; PARG [poly (ADP-ribose) glycohydrolase]; PARK2 [Parkinson disease (autosomal recessive, juvenile) 2, parkin]; PARP1 [poly (ADP-ribose) polymerase 1]; PAWR [PRKC, apoptosis, WT1, regulator]; PAX2 [paired box 2]; PAX3 [paired box 3]; PAX5 [paired box 5]; PAX6 [paired box 6]; PAXIP1 [PAX interacting (with transcription-activation domain) protein 1]; PC [pyruvate carboxylase]; PCCA [propionyl Coenzyme A carboxylase, alpha polypeptide]; PCCB [propionyl Coenzyme A carboxylase, beta polypeptide]; PCDH1 [protocadherin 1]; PCK1 [phosphoenolpyruvate carboxykinase 1 (soluble)]; PCM1 [pericentriolar material 1]; PCNA [proliferating cell nuclear antigen]; PCNT [pericentrin]; PCSK1 [proprotein convertase subtilisin/kexin type 1]; PCSK6 [proprotein convertase subtilisin/kexin type 6]; PCSK7 [proprotein convertase subtilisin/kexin type 7]; PCYT1A [phosphate cytidylyltransferase 1, choline, alpha]; PCYT2 [phosphate cytidylyltransferase 2, ethanolamine]; PDCD1 [programmed cell death 1]; PDCD1LG2 [programmed cell death 1 ligand 2]; PDCD6 [programmed cell death 6]; PDE3B [phosphodiesterase 3B, cGMP-inhibited]; PDE4A [phosphodiesterase 4A, cAMP-specific (phosphodiesterase E2 dunce homolog, Drosophila)]; PDE4B [phosphodiesterase 4B, cAMP-specific (phosphodiesterase E4 dunce homolog, Drosophila)]; PDE4D [phosphodiesterase 4D, cAMP-specific (phosphodiesterase E3 dunce homolog, Drosophila)]; PDE7A [phosphodiesterase 7A]; PDGFA [platelet-derived growth factor alpha polypeptide]; PDGFB [platelet-derived growth factor beta polypeptide (simian sarcoma viral (v-sis) oncogene homolog)]; PDGFRA [platelet-derived growth factor receptor, alpha polypeptide]; PDGFRB [platelet-derived growth factor receptor, beta polypeptide]; PDIA2 [protein disulfide isomerase family A, member 2]; PDIA3 [protein disulfide isomerase family A, member 3]; PDK1 [pyruvate dehydrogenase kinase, isozyme 1]; PDLIM1 [PDZ and LIM domain 1]; PDLIM5 [PDZ and LIM domain 5]; PDLIM7 [PDZ and LIM domain 7 (enigma)]; PDP1 [pyruvate dehyrogenase phosphatase catalytic subunit 1]; PDX1 [pancreatic and duodenal homeobox 1]; PDXK [pyridoxal (pyridoxine, vitamin B6) kinase]; PDYN [prodynorphin]; PECAM1 [platelet/endothelial cell adhesion molecule]; PEMT [phosphatidylethanolamine N-methyltransferase]; PENK [proenkephalin]; PEPD [peptidase D]; PER1 [period homolog 1 (Drosophila)]; PEX1 [peroxisomal biogenesis factor 1]; PEX10 [peroxisomal biogenesis factor 10]; PEX12 [peroxisomal biogenesis factor 12]; PEX13 [peroxisomal biogenesis factor 13]; PEX14 [peroxisomal biogenesis factor 14]; PEX16 [peroxisomal biogenesis factor 16]; PEX19 [peroxisomal biogenesis factor 19]; PEX2 [peroxisomal biogenesis factor 2]; PEX26 [peroxisomal biogenesis factor 26]; PEX3 [peroxisomal biogenesis factor 3]; PEX5 [peroxisomal biogenesis factor 5]; PEX6 [peroxisomal biogenesis factor 6]; PEX7 [peroxisomal biogenesis factor 7]; PF4 [platelet factor 4]; PFAS [phosphoribosylformylglycinamidine synthase]; PFDN4 [prefoldin subunit 4]; PFN1 [profilin 1]; PGC [progastricsin (pepsinogen C)]; PGD [phosphogluconate dehydrogenase]; PGF [placental growth factor]; PGK1 [phosphoglycerate kinase 1]; PGM1 [phosphoglucomutase 1]; PGR [progesterone receptor]; PHB [prohibitin]; PHEX [phosphate regulating endopeptidase homolog, X-linked]; PHF11 [PHD finger protein 11]; PHOX2B [paired-like homeobox 2b]; PHTF1 [putative homeodomain transcription factor 1]; PHYH [phytanoyl-CoA 2-hydroxylase]; PHYHIP [phytanoyl-CoA 2-hydroxylase interacting protein]; PI3 [peptidase inhibitor 3, skin-derived]; PIGA [phosphatidylinositol glycan anchor biosynthesis, class A]; PIGR [polymeric immunoglobulin receptor]; PIK3C2A [phosphoinositide-3-kinase, class 2, alpha polypeptide]; PIK3C2B [phosphoinositide-3-kinase, class 2, beta polypeptide]; PIK3C2G [phosphoinositide-3-kinase, class 2, gamma polypeptide]; PIK3C3 [phosphoinositide-3-kinase, class 3]; PIK3CA [phosphoinositide-3-kinase, catalytic, alpha polypeptide]; PIK3CB [phosphoinositide-3-kinase, catalytic, beta polypeptide]; PIK3CD [phosphoinositide-3-kinase, catalytic, delta polypeptide]; PIK3CG [phosphoinositide-3-kinase, catalytic, gamma polypeptide]; PIK3R1 [phosphoinositide-3-kinase, regulatory subunit 1 (alpha)]; PIK3R2 [phosphoinositide-3-kinase, regulatory subunit 2 (beta)]; PIK3R3 [phosphoinositide-3-kinase, regulatory subunit 3 (gamma)]; PIKFYVE [phosphoinositide kinase, FYVE finger containing]; PIN1 [peptidylprolyl cis/trans isomerase, NIMA-interacting 1]; PINK1 [PTEN induced putative kinase 1]; PIP [prolactin-induced protein]; PIP5KL1 [phosphatidylinositol-4-phosphate 5-kinase-like 1]; PITPNM1 [phosphatidylinositol transfer protein, membrane-associated 1]; PITRM1 [pitrilysin metallopeptidase 1]; PITX2 [paired-like homeodomain 2]; PKD2 [polycystic kidney disease 2 (autosomal dominant)]; PKLR [pyruvate kinase, liver and RBC]; PKM2 [pyruvate kinase, muscle]; PKN1 [protein kinase N1]; PL-5283 [PL-5283 protein]; PLA2G1B [phospholipase A2, group IB (pancreas)]; PLA2G2A [phospholipase A2, group IIA (platelets, synovial fluid)]; PLA2G2D [phospholipase A2, group IID]; PLA2G4A [phospholipase A2, group IVA (cytosolic, calcium-dependent)]; PLA2G6 [phospholipase A2, group VI (cytosolic, calcium-independent)]; PLA2G7 [phospholipase A2, group VII (platelet-activating factor acetylhydrolase, plasma)]; PLA2R1 [phospholipase A2 receptor 1, 180 kDa]; PLAT [plasminogen activator, tissue]; PLAU [plasminogen activator, urokinase]; PLAUR [plasminogen activator, urokinase receptor]; PLCB1 [phospholipase C, beta 1 (phosphoinositide-specific)]; PLCB2 [phospholipase C, beta 2]; PLCB4 [phospholipase C, beta 4]; PLCD1 [phospholipase C, delta 1]; PLCG1 [phospholipase C, gamma 1]; PLCG2 [phospholipase C, gamma 2 (phosphatidylinositol-specific)]; PLD1 [phospholipase D1, phosphatidylcholine-specific]; PLEC [plectin]; PLEK [pleckstrin]; PLG [plasminogen]; PLIN1 [perilipin 1]; PLK1 [polo-like kinase 1 (Drosophila)]; PLK2 [polo-like kinase 2 (Drosophila)]; PLK3 [polo-like kinase 3 (Drosophila)]; PLP1 [proteolipid protein 1]; PLTP [phospholipid transfer protein]; PMAIP1 [phorbol-12-myristate-13-acetate-induced protein 1]; PMCH [pro-melanin-concentrating hormone]; PML [promyelocytic leukemia]; PMP22 [peripheral myelin protein 22]; PMS2 [PMS2 postmeiotic segregation increased 2 (S. cerevisiae)]; PNLIP [pancreatic lipase]; PNMA3 [paraneoplastic antigen MA3]; PNMT [phenylethanolamine N-methyltransferase]; PNP [purine nucleoside phosphorylase]; POLB [polymerase (DNA directed), beta]; POLD3 [polymerase (DNA-directed), delta 3, accessory subunit]; POLD4 [polymerase (DNA-directed), delta 4]; POLH [polymerase (DNA directed), eta]; POLL [polymerase (DNA directed), lambda]; POLR2A [polymerase (RNA) II (DNA directed) polypeptide A, 220 kDa]; POLR2B [polymerase (RNA) II (DNA directed) polypeptide B, 140 kDa]; POLR2c [polymerase (RNA) II (DNA directed) polypeptide C, 33 kDa]; POLR2D [polymerase (RNA) II (DNA directed) polypeptide D]; POLR2E [polymerase (RNA) II (DNA directed) polypeptide E, 25 kDa]; POLR2F [polymerase (RNA) II (DNA directed) polypeptide F]; POLR2G [polymerase (RNA) II (DNA directed) polypeptide G]; POLR2H [polymerase (RNA) II (DNA directed) polypeptide H]; POLR2I [polymerase (RNA) II (DNA directed) polypeptide 1, 14.5 kDa]; POLR2J [polymerase (RNA) II (DNA directed) polypeptide J, 13.3 kDa]; POLR2K [polymerase (RNA) II (DNA directed) polypeptide K, 7.0 kDa]; POLR2L [polymerase (RNA) II (DNA directed) polypeptide L, 7.6 kDa]; POMC [proopiomelanocortin]; POMT1 [protein-O-mannosyltransferase 1]; PON1 [paraoxonase 1]; PON2 [paraoxonase 2]; PON3 [paraoxonase 3]; POSTN [periostin, osteoblast specific factor]; POT1 [POT1 protection of telomeres 1 homolog (S. pombe)]; POU2AF1 [POU class 2 associating factor 1]; POU2F1 [POU class 2 homeobox 1]; POU2F2 [POU class 2 homeobox 2]; POU5F1 [POU class 5 homeobox 1]; PPA1 [pyrophosphatase (inorganic) 1]; PPARA [peroxisome proliferator-activated receptor alpha]; PPARD [peroxisome proliferator-activated receptor delta]; PPARG [peroxisome proliferator-activated receptor gamma]; PPARGC1A [peroxisome proliferator-activated receptor gamma, coactivator 1 alpha]; PPAT [phosphoribosyl pyrophosphate amidotransferase]; PPBP [pro-platelet basic protein (chemokine (C-X-C motif) ligand 7)]; PPFIA1 [protein tyrosine phosphatase, receptor type, f polypeptide (PTPRF), interacting protein (liprin), alpha 1]; PPIA [peptidylprolyl isomerase A (cyclophilin A)]; PPIB [peptidylprolyl isomerase B (cyclophilin B)]; PPIG [peptidylprolyl isomerase G (cyclophilin G)]; PPDX [protoporphyrinogen oxidase]; PPP1CB [protein phosphatase 1, catalytic subunit, beta isozyme]; PPP1R12A [protein phosphatase 1, regulatory (inhibitor) subunit 12A]; PPP1R2 [protein phosphatase 1, regulatory (inhibitor) subunit 2]; PPP2R1B [protein phosphatase 2, regulatory subunit A, beta]; PPP2R2B [protein phosphatase 2, regulatory subunit B, beta]; PPP2R4 [protein phosphatase 2A activator, regulatory subunit 4]; PPP6C [protein phosphatase 6, catalytic subunit]; PPT1 [palmitoyl-protein thioesterase 1]; PPY [pancreatic polypeptide]; PRDM1 [PR domain containing 1, with ZNF domain]; PRDM2 [PR domain containing 2, with ZNF domain]; PRDX2 [peroxiredoxin 2]; PRDX3 [peroxiredoxin 3]; PRDX5 [peroxiredoxin 5]; PRF1 [perforin 1 (pore forming protein)]; PRG2 [proteoglycan 2, bone marrow (natural killer cell activator, eosinophil granule major basic protein)]; PRG4 [proteoglycan 4]; PRIM1 [primase, DNA, polypeptide 1 (49 kDa)]; PRKAA1 [protein kinase, AMP-activated, alpha 1 catalytic subunit]; PRKAA2 [protein kinase, AMP-activated, alpha 2 catalytic subunit]; PRKAB1 [protein kinase, AMP-activated, beta 1 non-catalytic subunit]; PRKACA [protein kinase, cAMP-dependent, catalytic, alpha]; PRKACB [protein kinase, cAMP-dependent, catalytic, beta]; PRKACG [protein kinase, cAMP-dependent, catalytic, gamma]; PRKAR1A [protein kinase, cAMP-dependent, regulatory, type I, alpha (tissue specific extinguisher 1)]; PRKAR2A [protein kinase, cAMP-dependent, regulatory, type II, alpha]; PRKAR2B [protein kinase, cAMP-dependent, regulatory, type II, beta]; PRKCA [protein kinase C, alpha]; PRKCB [protein kinase C, beta]; PRKCD [protein kinase C, delta]; PRKCE [protein kinase C, epsilon]; PRKCG [protein kinase C, gamma]; PRKCH [protein kinase C, eta]; PRKCI [protein kinase C, iota]; PRKCQ [protein kinase C, theta]; PRKCZ [protein kinase C, zeta]; PRKD1 [protein kinase D1]; PRKD3 [protein kinase D3]; PRKDC [protein kinase, DNA-activated, catalytic polypeptide; also known as DNAPK]; PRKG1 [protein kinase, cGMP-dependent, type I]; PRKRIR [protein-kinase, interferon-inducible double stranded RNA dependent inhibitor, repressor of (P58 repressor)]; PRL [prolactin]; PRLR [prolactin receptor]; PRNP [prion protein]; PROC [protein C (inactivator of coagulation factors Va and VIIIa)]; PRODH [proline dehydrogenase (oxidase) 1]; PROK1 [prokineticin 1]; PROK2 [prokineticin 2]; PROM1 [prominin 1]; PRO51 [protein S (alpha)]; PRPH [peripherin]; PRSS1 [protease, serine, 1 (trypsin 1)]; PRSS2 [protease, serine, 2 (trypsin 2)]; PRSS21 [protease, serine, 21 (testisin)]; PRSS3 [protease, serine, 3]; PRTN3 [proteinase 3]; PSAP [prosaposin]; PSEN1 [presenilin 1]; PSEN2 [presenilin 2 (Alzheimer disease 4)]; PSMA1 [proteasome (prosome, macropain) subunit, alpha type, 1]; PSMA2 [proteasome (prosome, macropain) subunit, alpha type, 2]; PSMA3 [proteasome (prosome, macropain) subunit, alpha type, 3]; PSMA5 [proteasome (prosome, macropain) subunit, alpha type, 5]; PSMA6 [proteasome (prosome, macropain) subunit, alpha type, 6]; PSMA7 [proteasome (prosome, macropain) subunit, alpha type, 7]; PSMB10 [proteasome (prosome, macropain) subunit, beta type, 10]; PSMB2 [proteasome (prosome, macropain) subunit, beta type, 2]; PSMB4 [proteasome (prosome, macropain) subunit, beta type, 4]; PSMB5 [proteasome (prosome, macropain) subunit, beta type, 5]; PSMB6 [proteasome (prosome, macropain) subunit, beta type, 6]; PSMB8 [proteasome (prosome, macropain) subunit, beta type, 8 (large multifunctional peptidase 7)]; PSMB9 [proteasome (prosome, macropain) subunit, beta type, 9 (large multifunctional peptidase 2)]; PSMC3 [proteasome (prosome, macropain) 26S subunit, ATPase, 3]; PSMC4 [proteasome (prosome, macropain) 26S subunit, ATPase, 4]; PSMC6 [proteasome (prosome, macropain) 26S subunit, ATPase, 6]; PSMD4 [proteasome (prosome, macropain) 26S subunit, non-ATPase, 4]; PSMD9 [proteasome (prosome, macropain) 26S subunit, non-ATPase, 9]; PSME1 [proteasome (prosome, macropain) activator subunit 1 (PA28 alpha)]; PSME3 [proteasome (prosome, macropain) activator subunit 3 (PA28 gamma; Ki)]; PSMG2 [proteasome (prosome, macropain) assembly chaperone 2]; PSORS1C1 [psoriasis susceptibility 1 candidate 1]; PSTPIP1 [proline-serine-threonine phosphatase interacting protein 1]; PTAFR [platelet-activating factor receptor]; PTBP1 [polypyrimidine tract binding protein 1]; PTCH1 [patched homolog 1 (Drosophila)]; PTEN [phosphatase and tensin homolog]; PTGDR [prostaglandin D2 receptor (DP)]; PTGDS [prostaglandin D2 synthase 21 kDa (brain)]; PTGER1 [prostaglandin E receptor 1 (subtype EP1), 42 kDa]; PTGER2 [prostaglandin E receptor 2 (subtype EP2), 53 kDa]; PTGER3 [prostaglandin E receptor 3 (subtype EP3)]; PTGER4 [prostaglandin E receptor 4 (subtype EP4)]; PTGES [prostaglandin E synthase]; PTGFR [prostaglandin F receptor (FP)]; PTGIR [prostaglandin 12 (prostacyclin) receptor (IP)]; PTGS1 [prostaglandin-endoperoxide synthase 1 (prostaglandin G/H synthase and cyclooxygenase)]; PTGS2 [prostaglandin-endoperoxide synthase 2 (prostaglandin G/H synthase and cyclooxygenase)]; PTH [parathyroid hormone]; PTHLH [parathyroid hormone-like hormone]; PTK2 [PTK2 protein tyrosine kinase 2]; PTK2B [PTK2B protein tyrosine kinase 2 beta]; PTK7 [PTK7 protein tyrosine kinase 7]; PTMS [parathymosin]; PTN [pleiotrophin]; PTPN1 [protein tyrosine phosphatase, non-receptor type 1]; PTPN11 [protein tyrosine phosphatase, non-receptor type 11]; PTPN12 [protein tyrosine phosphatase, non-receptor type 12]; PTPN2 [protein tyrosine phosphatase, non-receptor type 2]; PTPN22 [protein tyrosine phosphatase, non-receptor type 22 (lymphoid)]; PTPN6 [protein tyrosine phosphatase, non-receptor type 6]; PTPRC [protein tyrosine phosphatase, receptor type, C]; PTPRD [protein tyrosine phosphatase, receptor type, D]; PTPRE [protein tyrosine phosphatase, receptor type, E]; PTPRJ [protein tyrosine phosphatase, receptor type, J]; PTPRN [protein tyrosine phosphatase, receptor type, N]; PTPRT [protein tyrosine phosphatase, receptor type, T]; PTPRU [protein tyrosine phosphatase, receptor type, U]; PTRF [polymerase I and transcript release factor]; PTS [6-pyruvoyltetrahydropterin synthase]; PTTG1 [pituitary tumor-transforming 1]; PTX3 [pentraxin 3, long]; PUS10 [pseudouridylate synthase 10]; PXK [PX domain containing serine/threonine kinase]; PXN [paxillin]; PYCR1 [pyrroline-5-carboxylate reductase 1]; PYCR2 [pyrroline-5-carboxylate reductase family, member 2]; PYGB [phosphorylase, glycogen; brain]; PYGM [phosphorylase, glycogen, muscle]; PYY [peptide YY]; PZP [pregnancy-zone protein]; QDPR [quinoid dihydropteridine reductase]; RAB11A [RAB11A, member RAS oncogene family]; RAB11FIP1 [RAB11 family interacting protein 1 (class I)]; RAB27A [RAB27A, member RAS oncogene family]; RAB37 [RAB37, member RAS oncogene family]; RAB39 [RAB39, member RAS oncogene family]; RAB7A [RAB7A, member RAS oncogene family]; RAB9A [RAB9A, member RAS oncogene family]; RAC1 [ras-related C3 botulinum toxin substrate 1 (rho family, small GTP binding protein Rac1)]; RAC2 [ras-related C3 botulinum toxin substrate 2 (rho family, small GTP binding protein Rac2)]; RAD17 [RAD17 homolog (S. pombe)]; RAD50 [RAD50 homolog (S. cerevisiae)]; RAD51 [RAD51 homolog (RecA homolog, E. coli) (S. cerevisiae)]; RAD51C [RAD51 homolog C (S. cerevisiae)]; RAD51L1 [RAD51-like 1 (S. cerevisiae)]; RAD51L3 [RAD51-like 3 (S. cerevisiae)]; RAD54L [RAD54-like (S. cerevisiae)]; RAD9A [RAD9 homolog A (S. pombe)]; RAF1 [v-raf-1 murine leukemia viral oncogene homolog 1]; RAG1 [recombination activating gene 1]; RAC2 [recombination activating gene 2]; RAN [RAN, member RAS oncogene family]; RANBP1 [RAN binding protein 1]; RAP1A [RAP1A, member of RAS oncogene family]; RAPGEF4 [Rap guanine nucleotide exchange factor (GEF) 4]; RARA [retinoic acid receptor, alpha]; RARB [retinoic acid receptor, beta]; RARG [retinoic acid receptor, gamma]; RARRES2 [retinoic acid receptor responder (tazarotene induced) 2]; RARS [arginyl-tRNA synthetase]; RASA1 [RAS p21 protein activator (GTPase activating protein) 1]; RASGRP1 [RAS guanyl releasing protein 1 (calcium and DAG-regulated)]; RASGRP2 [RAS guanyl releasing protein 2 (calcium and DAG-regulated)]; RASGRP4 [RAS guanyl releasing protein 4]; RASSF1 [Ras association (RalGDS/AF-6) domain family member 1]; RB1 [retinoblastoma 1]; RBBP4 [retinoblastoma binding protein 4]; RBBP8 [retinoblastoma binding protein 8]; RBL1 [retinoblastoma-like 1 (p107)]; RBL2 [retinoblastoma-like 2 (p130)]; RBP4 [retinol binding protein 4, plasma]; RBX1 [ring-box 1]; RCBTB1 [regulator of chromosome condensation (RCC1) and BTB (POZ) domain containing protein 1]; RCN1 [reticulocalbin 1, EF-hand calcium binding domain]; RCN2 [reticulocalbin 2, EF-hand calcium binding domain]; RDX [radixin]; RECK [reversion-inducing-cysteine-rich protein with kazal motifs]; RECQL [RecQ protein-like (DNA helicase Q1-like)]; RECQL4 [RecQ protein-like 4]; RECQL5 [RecQ protein-like 5]; REG1A [regenerating islet-derived 1 alpha]; REG3A [regenerating islet-derived 3 alpha]; REG4 [regenerating islet-derived family, member 4]; REL [v-rel reticuloendotheliosis viral oncogene homolog (avian)]; RELA [v-rel reticuloendotheliosis viral oncogene homolog A (avian)]; RELB [v-rel reticuloendotheliosis viral oncogene homolog B]; REN [renin]; RET [ret proto-oncogene]; RETN [resistin]; RETNLB [resistin like beta]; RFC1 [replication factor C (activator 1) 1, 145 kDa]; RFC2 [replication factor C (activator 1) 2, 40 kDa]; RFC3 [replication factor C (activator 1) 3, 38 kDa]; RFX1 [regulatory factor X, 1 (influences HLA class II expression)]; RFX5 [regulatory factor X, 5 (influences HLA class II expression)]; RFXANK [regulatory factor X-associated ankyrin-containing protein]; RFXAP [regulatory factor X-associated protein]; RGS18 [regulator of G-protein signaling 18]; RHAG [Rh-associated glycoprotein]; RHD [Rh blood group, D antigen]; RHO [rhodopsin]; RHOA [ras homolog gene family, member A]; RHOD [ras homolog gene family, member D]; RIF1 [RAP1 interacting factor homolog (yeast)]; RIPK1 [receptor (TNFRSF)-interacting serine-threonine kinase 1]; RIPK2 [receptor-interacting serine-threonine kinase 2]; RLBP1 [retinaldehyde binding protein 1]; RLN1 [relaxin 1]; RLN2 [relaxin 2]; RMI1 [RMI1, RecQ mediated genome instability 1, homolog (S. cerevisiae)]; RNASE1 [ribonuclease, RNase A family, 1 (pancreatic)]; RNASE2 [ribonuclease, RNase A family, 2 (liver, eosinophil-derived neurotoxin)]; RNASE3 [ribonuclease, RNase A family, 3 (eosinophil cationic protein)]; RNASEH1 [ribonuclease H1]; RNASEH2A [ribonuclease H2, subunit A]; RNASEL [ribonuclease L (2′ [5′-oligoisoadenylate synthetase-dependent)]; RNASEN [ribonuclease type III, nuclear]; RNF123 [ring finger protein 123]; RNF13 [ring finger protein 13]; RNF135 [ring finger protein 135]; RNF138 [ring finger protein 138]; RNF4 [ring finger protein 4]; RNH1 [ribonuclease/angiogenin inhibitor 1]; RNPC3 [RNA-binding region (RNP1, RRM) containing 3]; RNPEP [arginyl aminopeptidase (aminopeptidase B)]; ROCK1 [Rho-associated, coiled-coil containing protein kinase 1]; ROM1 [retinal outer segment membrane protein 1]; ROR2 [receptor tyrosine kinase-like orphan receptor 2]; RORA [RAR-related orphan receptor A]; RPA1 [replication protein A1, 70 kDa]; RPA2 [replication protein A2, 32 kDa]; RPGRIP1L [RPGRIP1-like]; RPLP1 [ribosomal protein, large, P1]; RPS19 [ribosomal protein S19]; RPS6KA3 [ribosomal protein S6 kinase, 90 kDa, polypeptide 3]; RPS6KB1 [ribosomal protein S6 kinase, 70 kDa, polypeptide 1]; RPSA [ribosomal protein SA]; RRBP1 [ribosome binding protein 1 homolog 180 kDa (dog)]; RRM1 [ribonucleotide reductase M1]; RRM2B [ribonucleotide reductase M2B (TP53 inducible)]; RUNX1 [runt-related transcription factor 1]; RUNX3 [runt-related transcription factor 3]; RXRA [retinoid X receptor, alpha]; RXRB [retinoid X receptor, beta]; RYR1 [ryanodine receptor 1 (skeletal)]; RYR3 [ryanodine receptor 3]; S100A1 [S100 calcium binding protein A1]; S100A12 [S100 calcium binding protein A12]; S100A4 [S100 calcium binding protein A4]; S100A7 [S100 calcium binding protein A7]; S100A8 [S100 calcium binding protein A8]; S100A9 [S100 calcium binding protein A9]; S100B [S100 calcium binding protein B]; S100G [S100 calcium binding protein G]; S1PR1 [sphingosine-1-phosphate receptor 1]; SAA1 [serum amyloid A1]; SAA4 [serum amyloid A4, constitutive]; SAFB [scaffold attachment factor B]; SAG [S-antigen; retina and pineal gland (arrestin)]; SAGE1 [sarcoma antigen 1]; SARDH [sarcosine dehydrogenase]; SART3 [squamous cell carcinoma antigen recognized by T cells 3]; SBDS [Shwachman-Bodian-Diamond syndrome]; SBNO2 [strawberry notch homolog 2 (Drosophila)]; SCAMP3 [secretory carrier membrane protein 3]; SOAP [SREBF chaperone]; SCARB1 [scavenger receptor class B, member 1]; SCD [stearoyl-CoA desaturase (delta-9-desaturase)]; SCG2 [secretogranin II]; SCG3 [secretogranin III]; SCG5 [secretogranin V (7B2 protein)]; SCGB1A1 [secretoglobin, family 1A, member 1 (uteroglobin)]; SCGB3A2 [secretoglobin, family 3A, member 2]; SCN4A [sodium channel, voltage-gated, type IV, alpha subunit]; SCNN1A [sodium channel, nonvoltage-gated 1 alpha]; SCNN1G [sodium channel, nonvoltage-gated 1, gamma]; SCO1 [SCO cytochrome oxidase deficient homolog 1 (yeast)]; SCO2 [SCO cytochrome oxidase deficient homolog 2 (yeast)]; SCP2 [sterol carrier protein 2]; SCT [secretin]; SDC1 [syndecan 1]; SDC2 [syndecan 2]; SDC4 [syndecan 4]; SDHB [succinate dehydrogenase complex, subunit B, iron sulfur (Ip)]; SDHD [succinate dehydrogenase complex, subunit D, integral membrane protein]; SEC14L2 [SEC14-like 2 (S. cerevisiae)]; SEC16A [SEC16 homolog A (S. cerevisiae)]; SEC23B [Sec23 homolog B (S. cerevisiae)]; SELE [selectin E]; SELL [selectin L]; SELP [selectin P (granule membrane protein 140 kDa, antigen CD62)]; SELPLG [selectin P ligand]; SEPT5 [septin 5]; SEPP1 [selenoprotein P, plasma, 1]; SEPSECS [Sep (O-phosphoserine) tRNA:Sec (selenocysteine) tRNA synthase]; SERBP1 [SERPINE1 mRNA binding protein 1]; SERPINA1 [serpin peptidase inhibitor, clade A (alpha-1 antiproteinase, antitrypsin), member 1]; SERPINA2 [serpin peptidase inhibitor, clade A (alpha-1 antiproteinase, antitrypsin), member 2]; SERPINA3 [serpin peptidase inhibitor, clade A (alpha-1 antiproteinase, antitrypsin), member 3]; SERPINA5 [serpin peptidase inhibitor, clade A (alpha-1 antiproteinase, antitrypsin), member 5]; SERPINA6 [serpin peptidase inhibitor, clade A (alpha-1 antiproteinase, antitrypsin), member 6]; SERPINA7 [serpin peptidase inhibitor, clade A (alpha-1 antiproteinase, antitrypsin), member 7]; SERPINB1 [serpin peptidase inhibitor, clade B (ovalbumin), member 1]; SERPINB2 [serpin peptidase inhibitor, clade B (ovalbumin), member 2]; SERPINB3 [serpin peptidase inhibitor, clade B (ovalbumin), member 3]; SERPINB4 [serpin peptidase inhibitor, clade B (ovalbumin), member 4]; SERPINB5 [serpin peptidase inhibitor, clade B (ovalbumin), member 5]; SERPINB6 [serpin peptidase inhibitor, clade B (ovalbumin), member 6]; SERPINB9 [serpin peptidase inhibitor, clade B (ovalbumin), member 9]; SERPINC1 [serpin peptidase inhibitor, clade C (antithrombin), member 1]; SERPIND1 [serpin peptidase inhibitor, clade D (heparin cofactor), member 1]; SERPINE1 [serpin peptidase inhibitor, clade E (nexin, plasminogen activator inhibitor type 1), member 1]; SERPINE2 [serpin peptidase inhibitor, clade E (nexin, plasminogen activator inhibitor type 1), member 2]; SERPINF2 [serpin peptidase inhibitor, clade F (alpha-2 antiplasmin, pigment epithelium derived factor), member 2]; SERPING1 [serpin peptidase inhibitor, clade G (C1 inhibitor), member 1]; SERPINH1 [serpin peptidase inhibitor, clade H (heat shock protein 47), member 1, (collagen binding protein 1)]; SET [SET nuclear oncogene]; SETDB2 [SET domain, bifurcated 2]; SETX [senataxin]; SFPQ [splicing factor proline/glutamine-rich (polypyrimidine tract binding protein associated)]; SFRP1 [secreted frizzled-related protein 1]; SFRP2 [secreted frizzled-related protein 2]; SFRP5 [secreted frizzled-related protein 5]; SFTPA1 [surfactant protein A1]; SFTPB [surfactant protein B]; SFTPC [surfactant protein C]; SFTPD [surfactant protein D]; SGCA [sarcoglycan, alpha (50 kDa dystrophin-associated glycoprotein)]; SGCB [sarcoglycan, beta (43 kDa dystrophin-associated glycoprotein)]; SGK1 [serum/glucocorticoid regulated kinase 1]; SGSH [N-sulfoglucosamine sulfohydrolase]; SGTA [small glutamine-rich tetratricopeptide repeat (TPR)-containing, alpha]; SH2B1 [SH2B adaptor protein 1]; SH2B3 [SH2B adaptor protein 3]; SH2D1A [SH2 domain containing 1A]; SH2D4B [SH2 domain containing 4B]; SH3KBP1 [SH3-domain kinase binding protein 1]; SHBG [sex hormone-binding globulin]; SHC1 [SHC (Src homology 2 domain containing) transforming protein 1]; SHH [sonic hedgehog homolog (Drosophila)]; SHMT2 [serine hydroxymethyltransferase 2 (mitochondrial)]; SI [sucrase-isomaltase (alpha-glucosidase)]; SIGIRR [single immunoglobulin and toll-interleukin 1 receptor (TIR) domain]; SIP1 [survival of motor neuron protein interacting protein 1]; SIPA1 [signal-induced proliferation-associated 1]; SIRPA [signal-regulatory protein alpha]; SIRPB2 [signal-regulatory protein beta 2]; SIRT1 [sirtuin (silent mating type information regulation 2 homolog) 1 (S. cerevisiae)]; SKIV2L [superkiller viralicidic activity 2-like (S. cerevisiae)]; SKP2 [S-phase kinase-associated protein 2 (p45)]; SLAMF1 [signaling lymphocytic activation molecule family member 1]; SLAMF6 [SLAM family member 6]; SLC11A1 [solute carrier family 11 (proton-coupled divalent metal ion transporters), member 1]; SLC11A2 [solute carrier family 11 (proton-coupled divalent metal ion transporters), member 2]; SLC12A1 [solute carrier family 12 (sodium/potassium/chloride transporters), member 1]; SLC12A2 [solute carrier family 12 (sodium/potassium/chloride transporters), member 2]; SLC14A1 [solute carrier family 14 (urea transporter), member 1 (Kidd blood group)]; SLC15A1 [solute carrier family 15 (oligopeptide transporter), member 1]; SLC16A1 [solute carrier family 16, member 1 (monocarboxylic acid transporter 1)]; SLC17A5 [solute carrier family 17 (anion/sugar transporter), member 5]; SLC17A6 [solute carrier family 17 (sodium-dependent inorganic phosphate cotransporter), member 6]; SLC17A7 [solute carrier family 17 (sodium-dependent inorganic phosphate cotransporter), member 7]; SLC19A1 [solute carrier family 19 (folate transporter), member 1]; SLC1A1 [solute carrier family 1 (neuronal/epithelial high affinity glutamate transporter, system Xag), member 1]; SLC1A2 [solute carrier family 1 (glial high affinity glutamate transporter), member 2]; SLC1A4 [solute carrier family 1 (glutamate/neutral amino acid transporter), member 4]; SLC22A12 [solute carrier family 22 (organic anion/urate transporter), member 12]; SLC22A2 [solute carrier family 22 (organic cation transporter), member 2]; SLC22A23 [solute carrier family 22, member 23]; SLC22A3 [solute carrier family 22 (extraneuronal monoamine transporter), member 3]; SLC22A4 [solute carrier family 22 (organic cation/ergothioneine transporter), member 4]; SLC22A5 [solute carrier family 22 (organic cation/carnitine transporter), member 5]; SLC22A6 [solute carrier family 22 (organic anion transporter), member 6]; SLC24A2 [solute carrier family 24 (sodium/potassium/calcium exchanger), member 2]; SLC25A1 [solute carrier family 25 (mitochondrial carrier; citrate transporter), member 1]; SLC25A20 [solute carrier family 25 (carnitine/acylcarnitine translocase), member 20]; SLC25A3 [solute carrier family 25 (mitochondrial carrier; phosphate carrier), member 3]; SLC25A32 [solute carrier family 25, member 32]; SLC25A33 [solute carrier family 25, member 33]; SLC25A4 [solute carrier family 25 (mitochondrial carrier; adenine nucleotide translocator), member 4]; SLC26A4 [solute carrier family 26, member 4]; SLC27A4 [solute carrier family 27 (fatty acid transporter), member 4]; SLC28A1 [solute carrier family 28 (sodium-coupled nucleoside transporter), member 1]; SLC2A1 [solute carrier family 2 (facilitated glucose transporter), member 1]; SLC2A13 [solute carrier family 2 (facilitated glucose transporter), member 13]; SLC2A3 [solute carrier family 2 (facilitated glucose transporter), member 3]; SLC2A4 [solute carrier family 2 (facilitated glucose transporter), member 4]; SLC30A1 [solute carrier family 30 (zinc transporter), member 1]; SLC30A8 [solute carrier family 30 (zinc transporter), member 8]; SLC31A1 [solute carrier family 31 (copper transporters), member 1]; SLC35A1 [solute carrier family 35 (CMP-sialic acid transporter), member A1]; SLC35A2 [solute carrier family 35 (UDP-galactose transporter), member A2]; SLC35C1 [solute carrier family 35, member C1]; SLC35F2 [solute carrier family 35, member F2]; SLC39A3 [solute carrier family 39 (zinc transporter), member 3]; SLC3A2 [solute carrier family 3 (activators of dibasic and neutral amino acid transport), member 2]; SLC46A1 [solute carrier family 46 (folate transporter), member 1]; SLC5A5 [solute carrier family 5 (sodium iodide symporter), member 5]; SLC6A11 [solute carrier family 6 (neurotransmitter transporter, GABA), member 11]; SLC6A14 [solute carrier family 6 (amino acid transporter), member 14]; SLC6A19 [solute carrier family 6 (neutral amino acid transporter), member 19]; SLC6A3 [solute carrier family 6 (neurotransmitter transporter, dopamine), member 3]; SLC6A4 [solute carrier family 6 (neurotransmitter transporter, serotonin), member 4]; SLC6A8 [solute carrier family 6 (neurotransmitter transporter, creatine), member 8]; SLC7A1 [solute carrier family 7 (cationic amino acid transporter, y+ system), member 1]; SLC7A2 [solute carrier family 7 (cationic amino acid transporter, y+ system), member 2]; SLC7A4 [solute carrier family 7 (cationic amino acid transporter, y+ system), member 4]; SLC7A5 [solute carrier family 7 (cationic amino acid transporter, y+ system), member 5]; SLC8A1 [solute carrier family 8 (sodium/calcium exchanger), member 1]; SLC9A1 [solute carrier family 9 (sodium/hydrogen exchanger), member 1]; SLC9A3R1 [solute carrier family 9 (sodium/hydrogen exchanger), member 3 regulator 1]; SLCO1A2 [solute carrier organic anion transporter family, member 1A2]; SLCO1B1 [solute carrier organic anion transporter family, member 1B1]; SLCO1B3 [solute carrier organic anion transporter family, member 1B3]; SLPI [secretory leukocyte peptidase inhibitor]; SMAD1 [SMAD family member 1]; SMAD2 [SMAD family member 2]; SMAD3 [SMAD family member 3]; SMAD4 [SMAD family member 4]; SMAD7 [SMAD family member 7]; SMARCA4 [SWI/SNF related, matrix associated, actin dependent regulator of chromatin, subfamily a, member 4]; SMARCAL1 [SWI/SNF related, matrix associated, actin dependent regulator of chromatin, subfamily a-like 1]; SMARCB1 [SWI/SNF related, matrix associated, actin dependent regulator of chromatin, subfamily b, member 1]; SMC1A [structural maintenance of chromosomes 1A]; SMC3 [structural maintenance of chromosomes 3]; SMG1 [SMG1 homolog, phosphatidylinositol 3-kinase-related kinase (C. elegans)]; SMN1 [survival of motor neuron 1, telomeric]; SMPD1 [sphingomyelin phosphodiesterase 1, acid lysosomal]; SMPD2 [sphingomyelin phosphodiesterase 2, neutral membrane (neutral sphingomyelinase)]; SMTN [smoothelin]; SNAI2 [snail homolog 2 (Drosophila)]; SNAP25 [synaptosomal-associated protein, 25 kDa]; SNCA [synuclein, alpha (non A4 component of amyloid precursor)]; SNCG [synuclein, gamma (breast cancer-specific protein 1)]; SNURF [SNRPN upstream reading frame]; SNW1 [SNW domain containing 1]; SNX9 [sorting nexin 9]; SOAT1 [sterol O-acyltransferase 1]; SOCS1 [suppressor of cytokine signaling 1]; SOCS2 [suppressor of cytokine signaling 2]; SOCS3 [suppressor of cytokine signaling 3]; SOD1 [superoxide dismutase 1, soluble]; SOD2 [superoxide dismutase 2, mitochondrial]; SORBS3 [sorbin and SH3 domain containing 3]; SORD [sorbitol dehydrogenase]; SOX2 [SRY (sex determining region Y)-box 2]; SP1 [Sp1 transcription factor]; SP110 [SP110 nuclear body protein]; SP3 [Sp3 transcription factor]; SPA17 [sperm autoantigenic protein 17]; SPARC [secreted protein, acidic, cysteine-rich (osteonectin)]; SPHK1 [sphingosine kinase 1]; SPI1 [spleen focus forming virus (SFFV) proviral integration oncogene spil]; SPINK1 [serine peptidase inhibitor, Kazal type 1]; SPINK13 [serine peptidase inhibitor, Kazal type 13 (putative)]; SPINK5 [serine peptidase inhibitor, Kazal type 5]; SPN [sialophorin]; SPON1 [spondin 1, extracellular matrix protein]; SPP1 [secreted phosphoprotein 1]; SPRED1 [sprouty-related, EVH1 domain containing 1]; SPRR2A [small proline-rich protein 2A]; SPRR2B [small proline-rich protein 2B]; SPTB [spectrin, beta, erythrocytic]; SRC [v-src sarcoma (Schmidt-Ruppin A-2) viral oncogene homolog (avian)]; SRD5A1 [steroid-5-alpha-reductase, alpha polypeptide 1 (3-oxo-5 alpha-steroid delta 4-dehydrogenase alpha 1)]; SREBF1 [sterol regulatory element binding transcription factor 1]; SREBF2 [sterol regulatory element binding transcription factor 2]; SRF [serum response factor (c-fos serum response element-binding transcription factor)]; SRGN [serglycin]; SRP9 [signal recognition particle 9 kDa]; SRPX [sushi-repeat-containing protein, X-linked]; SRR [serine racemase]; SRY [sex determining region Y]; SSB [Sjogren syndrome antigen B (autoantigen La)]; SST [somatostatin]; SSTR2 [somatostatin receptor 2]; SSTR4 [somatostatin receptor 4]; ST8SIA4 [ST8 alpha-N-acetyl-neuraminide alpha-2,8-sialyltransferase 4]; STAR [steroidogenic acute regulatory protein]; STAT1 [signal transducer and activator of transcription 1, 91 kDa]; STAT2 [signal transducer and activator of transcription 2, 113 kDa]; STAT3 [signal transducer and activator of transcription 3 (acute-phase response factor)]; STAT4 [signal transducer and activator of transcription 4]; STAT5A [signal transducer and activator of transcription 5A]; STAT5B [signal transducer and activator of transcription 5B]; STAT6 [signal transducer and activator of transcription 6, interleukin-4 induced]; STELLAR [germ and embryonic stem cell enriched protein STELLA]; STIM1 [stromal interaction molecule 1]; STIP1 [stress-induced-phosphoprotein 1]; STK11 [serine/threonine kinase 11]; STMN2 [stathmin-like 2]; STRAP [serine/threonine kinase receptor associated protein]; STRC [stereocilin]; STS [steroid sulfatase (microsomal), isozyme S]; STX6 [syntaxin 6]; STX8 [syntaxin 8]; SULT1A1 [sulfotransferase family, cytosolic, 1A, phenol-preferring, member 1]; SULT1A3 [sulfotransferase family, cytosolic, 1A, phenol-preferring, member 3]; SUMF1 [sulfatase modifying factor 1]; SUMO1 [SMT3 suppressor of mif two 3 homolog 1 (S. cerevisiae)]; SUMO3 [SMT3 suppressor of mif two 3 homolog 3 (S. cerevisiae)]; SUOX [sulfite oxidase]; SUV39H1 [suppressor of variegation 3-9 homolog 1 (Drosophila)]; SWAP70 [SWAP switching B-cell complex 70 kDa subunit]; SYCP3 [synaptonemal complex protein 3]; SYK [spleen tyrosine kinase]; SYNM [synemin, intermediate filament protein]; SYNPO [synaptopodin]; SYNPO2 [synaptopodin 2]; SYP [synaptophysin]; SYT3 [synaptotagmin III]; SYTL1 [synaptotagmin-like 1]; T [T, brachyury homolog (mouse)]; TAC1 [tachykinin, precursor 1]; TAC4 [tachykinin 4 (hemokinin)]; TACR1 [tachykinin receptor 1]; TACR2 [tachykinin receptor 2]; TACR3 [tachykinin receptor 3]; TAGLN [transgelin]; TAL1 [T-cell acute lymphocytic leukemia 1]; TAOK3 [TAO kinase 3]; TAP1 [transporter 1, ATP-binding cassette, sub-family B (MDR/TAP)]; TAP2 [transporter 2, ATP-binding cassette, sub-family B (MDR/TAP)]; TARDBP [TAR DNA binding protein]; TARP [TCR gamma alternate reading frame protein]; TAT [tyrosine aminotransferase]; TBK1 [TANK-binding kinase 1]; TBP [TATA box binding protein]; TBX1 [T-box 1]; TBX2 [T-box 2]; TBX21 [T-box 21]; TBX3 [T-box 3]; TBX5 [T-box 5]; TBXA2R [thromboxane A2 receptor]; TBXAS1 [thromboxane A synthase 1 (platelet)]; TCEA1 [transcription elongation factor A (SII), 1]; TCEAL1 [transcription elongation factor A (SII)-like 1]; TCF4 [transcription factor 4]; TCF7L2 [transcription factor 7-like 2 (T-cell specific, HMG-box)]; TCL1A [T-cell leukemia/lymphoma 1A]; TCL1B [T-cell leukemia/lymphoma 1B]; TCN1 [transcobalamin I (vitamin B12 binding protein, R binder family)]; TCN2 [transcobalamin II; macrocytic anemia]; TDP1 [tyrosyl-DNA phosphodiesterase 1]; TEC [tec protein tyrosine kinase]; TECTA [tectorin alpha]; TEK [TEK tyrosine kinase, endothelial]; TERF1 [telomeric repeat binding factor (NIMA-interacting) 1]; TERF2 [telomeric repeat binding factor 2]; TERT [telomerase reverse transcriptase]; TES [testis derived transcript (3 LIM domains)]; TF [transferrin]; TFAM [transcription factor A, mitochondrial]; TFAP2A [transcription factor AP-2 alpha (activating enhancer binding protein 2 alpha)]; TFF2 [trefoil factor 2]; TFF3 [trefoil factor 3 (intestinal)]; TFPI [tissue factor pathway inhibitor (lipoprotein-associated coagulation inhibitor)]; TFPT [TCF3 (E2A) fusion partner (in childhood Leukemia)]; TFR2 [transferrin receptor 2]; TFRC [transferrin receptor (p90, CD71)]; TG [thyroglobulin]; TGFA [transforming growth factor, alpha]; TGFB1 [transforming growth factor, beta 1]; TGFB2 [transforming growth factor, beta 2]; TGFB3 [transforming growth factor, beta 3]; TGFBR1 [transforming growth factor, beta receptor 1]; TGFBR2 [transforming growth factor, beta receptor II (70/80 kDa)]; TGIF1 [TGFB-induced factor homeobox 1]; TGM1 [transglutaminase 1 (K polypeptide epidermal type I, protein-glutamine-gamma-glutamyltransferase)]; TGM2 [transglutaminase 2 (C polypeptide, protein-glutamine-gamma-glutamyltransferase)]; TGM3 [transglutaminase 3 (E polypeptide, protein-glutamine-gamma-glutamyltransferase)]; TH [tyrosine hydroxylase]; THAP1 [THAP domain containing, apoptosis associated protein 1]; THBD [thrombomodulin]; THBS1 [thrombospondin 1]; THBS3 [thrombospondin 3]; THPO [thrombopoietin]; THY1 [Thy-1 cell surface antigen]; TIA1 [TIA1 cytotoxic granule-associated RNA binding protein]; TIE1 [tyrosine kinase with immunoglobulin-like and EGF-like domains 1]; TIMD4 [T-cell immunoglobulin and mucin domain containing 4]; TIMELESS [timeless homolog (Drosophila)]; TIMP1 [TIMP metallopeptidase inhibitor 1]; TIMP2 [TIMP metallopeptidase inhibitor 2]; TIMP3 [TIMP metallopeptidase inhibitor 3]; TIRAP [toll-interleukin 1 receptor (TIR) domain containing adaptor protein]; TJP1 [tight junction protein 1 (zona occludens 1)]; TK1 [thymidine kinase 1, soluble]; TK2 [thymidine kinase 2, mitochondrial]; TKT [transketolase]; TLE4 [transducin-like enhancer of split 4 (E(sp1) homolog, Drosophila)]; TLR1 [toll-like receptor 1]; TLR10 [toll-like receptor 10]; TLR2 [toll-like receptor 2]; TLR3 [toll-like receptor 3]; TLR4 [toll-like receptor 4]; TLR5 [toll-like receptor 5]; TLR6 [toll-like receptor 6]; TLR7 [toll-like receptor 7]; TLR8 [toll-like receptor 8]; TLR9 [toll-like receptor 9]; TLX1 [T-cell leukemia homeobox 1]; TM7SF4 [transmembrane 7 superfamily member 4]; TMED3 [transmembrane emp24 protein transport domain containing 3]; TMEFF2 [transmembrane protein with EGF-like and two follistatin-like domains 2]; TMEM132E [transmembrane protein 132E]; TMEM18 [transmembrane protein 18]; TMEM19 [transmembrane protein 19]; TMEM216 [transmembrane protein 216]; TMEM27 [transmembrane protein 27]; TMEM67 [transmembrane protein 67]; TMPO [thymopoietin]; TMPRSS15 [transmembrane protease, serine 15]; TMSB4X [thymosin beta 4, X-linked]; TNC [tenascin C]; TNF [tumor necrosis factor (TNF superfamily, member 2)]; TNFAIP1 [tumor necrosis factor, alpha-induced protein 1 (endothelial)]; TNFAIP3 [tumor necrosis factor, alpha-induced protein 3]; TNFAIP6 [tumor necrosis factor, alpha-induced protein 6]; TNFRSF10A [tumor necrosis factor receptor superfamily, member 10a]; TNFRSF10B [tumor necrosis factor receptor superfamily, member 10b]; TNFRSF100 [tumor necrosis factor receptor superfamily, member 10c, decoy without an intracellular domain]; TNFRSF10D [tumor necrosis factor receptor superfamily, member 10d, decoy with truncated death domain]; TNFRSF11A [tumor necrosis factor receptor superfamily, member 11a, NFKB activator]; TNFRSF11B [tumor necrosis factor receptor superfamily, member 11b]; TNFRSF13B [tumor necrosis factor receptor superfamily, member 13B]; TNFRSF130 [tumor necrosis factor receptor superfamily, member 13C]; TNFRSF14 [tumor necrosis factor receptor superfamily, member 14 (herpesvirus entry mediator)]; TNFRSF17 [tumor necrosis factor receptor superfamily, member 17]; TNFRSF18 [tumor necrosis factor receptor superfamily, member 18]; TNFRSF1A [tumor necrosis factor receptor superfamily, member 1A]; TNFRSF1B [tumor necrosis factor receptor superfamily, member 1B]; TNFRSF21 [tumor necrosis factor receptor superfamily, member 21]; TNFRSF25 [tumor necrosis factor receptor superfamily, member 25]; TNFRSF4 [tumor necrosis factor receptor superfamily, member 4]; TNFRSF6B [tumor necrosis factor receptor superfamily, member 6b, decoy]; TNFRSF8 [tumor necrosis factor receptor superfamily, member 8]; TNFRSF9 [tumor necrosis factor receptor superfamily, member 9]; TNFSF10 [tumor necrosis factor (ligand) superfamily, member 10]; TNFSF11 [tumor necrosis factor (ligand) superfamily, member 11]; TNFSF12 [tumor necrosis factor (ligand) superfamily, member 12]; TNFSF13 [tumor necrosis factor (ligand) superfamily, member 13]; TNFSF13B [tumor necrosis factor (ligand) superfamily, member 13b]; TNFSF14 [tumor necrosis factor (ligand) superfamily, member 14]; TNFSF15 [tumor necrosis factor (ligand) superfamily, member 15]; TNFSF18 [tumor necrosis factor (ligand) superfamily, member 18]; TNFSF4 [tumor necrosis factor (ligand) superfamily, member 4]; TNFSF8 [tumor necrosis factor (ligand) superfamily, member 8]; TNFSF9 [tumor necrosis factor (ligand) superfamily, member 9]; TNKS [tankyrase, TRF1-interacting ankyrin-related ADP-ribose polymerase]; TNNC1 [troponin C type 1 (slow)]; TNNI2 [troponin I type 2 (skeletal, fast)]; TNNI3 [troponin I type 3 (cardiac)]; TNNT3 [troponin T type 3 (skeletal, fast)]; TNPO1 [transportin 1]; TNS1 [tensin 1]; TNXB [tenascin XB]; TOM1L2 [target of myb1-like 2 (chicken)]; TOP1 [topoisomerase (DNA) I]; TOP1MT [topoisomerase (DNA) I, mitochondrial]; TOP2A [topoisomerase (DNA) II alpha 170 kDa]; TOP2B [topoisomerase (DNA) II beta 180 kDa]; TOP3A [topoisomerase (DNA) III alpha]; TOPBP1 [topoisomerase (DNA) II binding protein 1]; TP53 [tumor protein p53]; TP53BP1 [tumor protein p53 binding protein 1]; TP53RK [TP53 regulating kinase]; TP63 [tumor protein p63]; TP73 [tumor protein p73]; TPD52 [tumor protein D52]; TPH1 [tryptophan hydroxylase 1]; TPI1 [triosephosphate isomerase 1]; TPM1 [tropomyosin 1 (alpha)]; TPM2 [tropomyosin 2 (beta)]; TPMT [thiopurine S-methyltransferase]; TPO [thyroid peroxidase]; TPP1 [tripeptidyl peptidase I]; TPP2 [tripeptidyl peptidase II]; TPPP [tubulin polymerization promoting protein]; TPPP3 [tubulin polymerization-promoting protein family member 3]; TPSAB1 [tryptase alpha/beta 1]; TPSB2 [tryptase beta 2 (gene/pseudogene)]; TPSD1 [tryptase delta 1]; TPSG1 [tryptase gamma 1]; TPT1 [tumor protein, translationally-controlled 1]; TRADD [TNFRSF1A-associated via death domain]; TRAF1 [TNF receptor-associated factor 1]; TRAF2 [TNF receptor-associated factor 2]; TRAF31P2 [TRAF3 interacting protein 2]; TRAF6 [TNF receptor-associated factor 6]; TRAIP [TRAF interacting protein]; TRAPPC10 [trafficking protein particle complex 10]; TRDN [triadin]; TREX1 [three prime repair exonuclease 1]; TRH [thyrotropin-releasing hormone]; TRIB1 [tribbles homolog 1 (Drosophila)]; TRIM21 [tripartite motif-containing 21]; TRIM22 [tripartite motif-containing 22]; TRIM26 [tripartite motif-containing 26]; TRIM28 [tripartite motif-containing 28]; TRIM29 [tripartite motif-containing 29]; TRIM68 [tripartite motif-containing 68]; TRPA1 [transient receptor potential cation channel, subfamily A, member 1]; TRPC1 [transient receptor potential cation channel, subfamily C, member 1]; TRPC3 [transient receptor potential cation channel, subfamily C, member 3]; TRPC6 [transient receptor potential cation channel, subfamily C, member 6]; TRPM1 [transient receptor potential cation channel, subfamily M, member 1]; TRPM8 [transient receptor potential cation channel, subfamily M, member 8]; TRPS1 [trichorhinophalangeal syndrome I]; TRPV1 [transient receptor potential cation channel, subfamily V, member 1]; TRPV4 [transient receptor potential cation channel, subfamily V, member 4]; TRPV5 [transient receptor potential cation channel, subfamily V, member 5]; TRPV6 [transient receptor potential cation channel, subfamily V, member 6]; TRRAP [transformation/transcription domain-associated protein]; TSC1 [tuberous sclerosis 1]; TSC2 [tuberous sclerosis 2]; TSC22D3 [TSC22 domain family, member 3]; TSG101 [tumor susceptibility gene 101]; TSHR [thyroid stimulating hormone receptor]; TSLP [thymic stromal lymphopoietin]; TSPAN7 [tetraspanin 7]; TSPO [translocator protein (18 kDa)]; TSSK2 [testis-specific serine kinase 2]; TSTA3 [tissue specific transplantation antigen P35B]; TTF2 [transcription termination factor, RNA polymerase II]; TTN [titin]; TTPA [tocopherol (alpha) transfer protein]; TTR [transthyretin]; TUBA1B [tubulin, alpha 1b]; TUBA4A [tubulin, alpha 4a]; TUBB [tubulin, beta]; TUBB1 [tubulin, beta 1]; TUBG1 [tubulin, gamma 1]; TWIST1 [twist homolog 1 (Drosophila)]; TWSG1 [twisted gastrulation homolog 1 (Drosophila)]; TXK [TXK tyrosine kinase]; TXN [thioredoxin]; TXN2 [thioredoxin 2]; TXNDC5 [thioredoxin domain containing 5 (endoplasmic reticulum)]; TXNDC9 [thioredoxin domain containing 9]; TXNIP [thioredoxin interacting protein]; TXNRD1 [thioredoxin reductase 1]; TXNRD2 [thioredoxin reductase 2]; TYK2 [tyrosine kinase 2]; TYMP [thymidine phosphorylase]; TYMS [thymidylate synthetase]; TYR [tyrosinase (oculocutaneous albinism IA)]; TYRO3 [TYRO3 protein tyrosine kinase]; TYROBP [TYRO protein tyrosine kinase binding protein]; TYRP1 [tyrosinase-related protein 1]; UBB [ubiquitin B]; UBC [ubiquitin C]; UBE2C [ubiquitin-conjugating enzyme E2C]; UBE2N [ubiquitin-conjugating enzyme E2N (UBC13 homolog, yeast)]; UBE2U [ubiquitin-conjugating enzyme E2U (putative)]; UBE3A [ubiquitin protein ligase E3A]; UBE4A [ubiquitination factor E4A (UFD2 homolog, yeast)]; UCHL1 [ubiquitin carboxyl-terminal esterase L1 (ubiquitin thiolesterase)]; UCN [urocortin]; UCN2 [urocortin 2]; UCP1 [uncoupling protein 1 (mitochondrial, proton carrier)]; UCP2 [uncoupling protein 2 (mitochondrial, proton carrier)]; UCP3 [uncoupling protein 3 (mitochondrial, proton carrier)]; UFD1L [ubiquitin fusion degradation 1 like (yeast)]; UGCG [UDP-glucose ceramide glucosyltransferase]; UGP2 [UDP-glucose pyrophosphorylase 2]; UGT1A1 [UDP glucuronosyltransferase 1 family, polypeptide A1]; UGT1A6 [UDP glucuronosyltransferase 1 family, polypeptide A6]; UGT1A7 [UDP glucuronosyltransferase 1 family, polypeptide A7]; UGT8 [UDP glycosyltransferase 8]; UIMC1 [ubiquitin interaction motif containing 1]; ULBP1 [UL16 binding protein 1]; ULK2 [unc-51-like kinase 2 (C. elegans)]; UMOD [uromodulin]; UMPS [uridine monophosphate synthetase]; UNC13D [unc-13 homolog D (C. elegans)]; UNC93B1 [unc-93 homolog B1 (C. elegans)]; UNG [uracil-DNA glycosylase]; UQCRFS1 [ubiquinol-cytochrome c reductase, Rieske iron-sulfur polypeptide 1]; UROD [uroporphyrinogen decarboxylase]; USF1 [upstream transcription factor 1]; USF2 [upstream transcription factor 2, c-fos interacting]; USP18 [ubiquitin specific peptidase 18]; USP34 [ubiquitin specific peptidase 34]; UTRN [utrophin]; UTS2 [urotensin 2]; VAMP8 [vesicle-associated membrane protein 8 (endobrevin)]; VAPA [VAMP (vesicle-associated membrane protein)-associated protein A, 33 kDa]; VASP [vasodilator-stimulated phosphoprotein]; VAV1 [vav 1 guanine nucleotide exchange factor]; VAV3 [vav 3 guanine nucleotide exchange factor]; VCAM1 [vascular cell adhesion molecule 1]; VCAN [versican]; VCL [vinculin]; VDAC1 [voltage-dependent anion channel 1]; VDR [vitamin D (1 [25-dihydroxyvitamin D3) receptor]; VEGFA [vascular endothelial growth factor A]; VEGFC [vascular endothelial growth factor C]; VHL [von Hippel-Lindau tumor suppressor]; VIL1 [villin 1]; VIM [vimentin]; VIP [vasoactive intestinal peptide]; VIPR1 [vasoactive intestinal peptide receptor 1]; VIPR2 [vasoactive intestinal peptide receptor 2]; VLDLR [very low density lipoprotein receptor]; VMAC [vimentin-type intermediate filament associated coiled-coil protein]; VPREB1 [pre-B lymphocyte 1]; VPS39 [vacuolar protein sorting 39 homolog (S. cerevisiae)]; VTN [vitronectin]; VWF [von Willebrand factor]; WARS [tryptophanyl-tRNA synthetase]; WAS [Wiskott-Aldrich syndrome (eczema-thrombocytopenia)]; WASF1 [WAS protein family, member 1]; WASF2 [WAS protein family, member 2]; WASL [Wiskott-Aldrich syndrome-like]; WDFY3 [WD repeat and FYVE domain containing 3]; WDR36 [WD repeat domain 36]; WEE1 [WEE1 homolog (S. pombe)]; WIF1 [WNT inhibitory factor 1]; WIPF1 [WAS/WASL interacting protein family, member 1]; WNK1 [WNK lysine deficient protein kinase 1]; WNT5A [wingless-type MMTV integration site family, member 5A]; WRN [Werner syndrome, RecQ helicase-like]; WT1 [Wilms tumor 1]; XBP1 [X-box binding protein 1]; XCL1 [chemokine (C motif) ligand 1]; XDH [xanthine dehydrogenase]; XIAP [X-linked inhibitor of apoptosis]; XPA [xeroderma pigmentosum, complementation group A]; XPC [xeroderma pigmentosum, complementation group C]; XPO5 [exportin 5]; XRCC1 [X-ray repair complementing defective repair in Chinese hamster cells 1]; XRCC2 [X-ray repair complementing defective repair in Chinese hamster cells 2]; XRCC3 [X-ray repair complementing defective repair in Chinese hamster cells 3]; XRCC4 [X-ray repair complementing defective repair in Chinese hamster cells 4]; XRCC5 [X-ray repair complementing defective repair in Chinese hamster cells 5 (double-strand-break rejoining)]; XRCC6 [X-ray repair complementing defective repair in Chinese hamster cells 6]; YAP1 [Yes-associated protein 1]; YARS [tyrosyl-tRNA synthetase]; YBX1 [Y box binding protein 1]; YES1 [v-yes-1 Yamaguchi sarcoma viral oncogene homolog 1]; YPEL1 [yippee-like 1 (Drosophila)]; YPEL2 [yippee-like 2 (Drosophila)]; YWHAB [tyrosine 3-monooxygenase/tryptophan 5-monooxygenase activation protein, beta polypeptide]; YWHAQ [tyrosine 3-monooxygenase/tryptophan 5-monooxygenase activation protein, theta polypeptide]; YWHAZ [tyrosine 3-monooxygenase/tryptophan 5-monooxygenase activation protein, zeta polypeptide]; YY1 [YY1 transcription factor]; ZAP70 [zeta-chain (TCR) associated protein kinase 70 kDa]; ZBED1 [zinc finger, BED-type containing 1]; ZC3H12A [zinc finger CCCH-type containing 12A]; ZC3H12D [zinc finger CCCH-type containing 12D]; ZFR [zinc finger RNA binding protein]; ZNF148 [zinc finger protein 148]; ZNF267 [zinc finger protein 267]; ZNF287 [zinc finger protein 287]; ZNF300 [zinc finger protein 300]; ZNF365 [zinc finger protein 365]; ZNF521 [zinc finger protein 521]; ZNF74 [zinc finger protein 74]; and ZPBP2 [zona pellucida binding protein 2].

In certain embodiments, an animal created by a method of the invention may be used to study the effects of mutations on the animal and development and/or progression of an immunodeficiency using measures commonly used in the study of immunodeficiencies.

It should be understood that the genetically modified animals, e.g., knock-out and transgenic animals created by a method of the invention may include genes altered singly or in combination, including alteration to any one or more of Rag1, Rag2, FoxN1, and DNAPK. Accordingly, for example, animals including a single, double or triple gene knock-out are contemplated. Any of these may be used in various methods in which alteration of one or more immunodeficiency genes may be useful. For example, genetically modified animals as described herein may be used in studies of hematopoietic cells, such as in the identification of progenitor cells including lymphoid progenitors and pluripotential stem cells; in the identification of new cytokines which play a role in the growth and differentiation of hematopoietic cells; in the analysis of the effect of known cytokines; and in the analysis of drugs effects on hematopoietic cells. Such animals can also be used in studies on pathogenetic mechanisms in disease caused by viral infections such as but not limited to influenza, West Nile virus, herpesviruses, picornaviruses, neurotropic coronavirus, Varicella-zoster (chicken pox), respiratory syncytial virus, cowpox, hepatitis B, rabies, and Dengue virus, and lymphotropic viruses including human immunodeficiency virus (HIV), human T lymphotropic virus (HTLV-1), and Epstein Barr virus (EBV), and also a virus that specifically infects rats but models the effects of a human-specific virus on its host, for example the rat-adapted influenza virus (see, e.g., H. Lebrec and G. R. Burleson (1994) Toxicology. July 1; 91(2):179-88).

In other embodiments, a genetically modified animal created by a method of the invention may also be useful in studies of defense mechanisms against microorganisms that cause disease in immunocompromised patients, wherein the microorganism may be cytomegalovirus, Pneumocystic carinii or Candida species. Genetically modified animals, such as for example knock-out rats can be subjects for pre-clinical evaluation of a specific “gene therapy”. For example, genes may be introduced into hematopoietic progenitor cells, preferably into pluripotential stem cells with self-renewal capacity from patients with inherited genetic defects, or into pluripotential stem cells with self-renewal capacity from rat models of inherited genetic defects, and the cells re-introduced into the genetically modified rats for the purpose of determining therapeutic usefulness of the modified cells. Genetically modified animals may also be useful for studying the biological mechanisms underlying immunodeficiency diseases and conditions caused by or linked to a mutation in an immunodeficiency gene such as Rag1, Rag2, FoxN1, or DNAPK.

Furthermore, a genetically modified animal created by a method of the invention may be used to develop a diagnostic assay for an immunodeficiency disorder including but not limited to a leukemia, in which the animal, either untreated or previously treated with a therapeutic agent, is assessed for the presence of one or more biomarkers relative to a non-affected control animal. Such a genetically modified animal may be used in a method of screening a candidate therapy or therapeutic compound for treating an immunodeficiency disorder such as a leukemia, using a genetically modified animal in which one or more immunodeficiency genes including but not limited to Rag1, Rag2, FoxN1, or DNAPK are knocked out, and the animal, either untreated or previously treated with another therapeutic agent which may be a drug, microbe, transplanted cells, or other agent, is then treated with the candidate therapy or candidate therapeutic agent, a biological sample is obtained from the animal, and the biological sample evaluated relative to a sample from a non-affected wild-type control sample, or a sample from a genetically modified animal not subjected to the candidate therapy or therapeutic agent.

In still further embodiments, a method for modeling an autoimmune disease may involve the adoptive transfer of B cells reacting to an antigen for an autoimmune disease, or T cells activated for an autoimmune disease. The appropriate non-human mammal with the antigen target of the autoimmune disease can be immunized as follows.

Immune cells may be prepared from the immunized animal and may be then transplanted to a genetically modified animal as described herein such as a Rag1, Rag2, FoxN1, or DNAPK knock-out rat, or a rat with any combination of these genes knocked out. The development of autoimmune phenotypes in the recipient knock-out animal may then evaluated as compared to either a non-transplanted knock-out animal, or as compared to a knock-out animal transplanted with non-pathologic immune cells that lack auto-reactivity, or as compared to a wild type animal transplanted with immune cells as described above.

In some embodiments, a method for creating a combined immunodeficiency syndrome model may include providing a genetically modified animal such as a rat wherein Rag1, Rag2, FoxN1, or DNAPK are knocked out as described herein, and the knock-out animal may be further rendered deficient for natural killer (NK) cells by any one of several possible methods. Non-limiting examples of methods of rendering the knock-out animal deficient for NK include i) disruption of the Lyst gene; or ii) treatment of FoxN1 mutant animals with a compound that inhibits NK cell activity including but not limited to NSAIDs (non-steroidal anti-inflammatory drugs), statins, allosteric LFA-1 inhibitors, vinblastine, paclitaxel, docetaxel, cladribine, chlorambucil, bortezomib, or MG-132.

### N. Trinucleotide Repeat Disorders

Trinucleotide repeat expansion disorders are divided into two categories determined by the type of repeat. The most common repeat is the triplet CAG, which, when present in the coding region of a gene, codes for the amino acid glutamine (Q). Therefore, these disorders are referred to as the polyglutamine (polyQ) disorders and may include Huntington Disease (HD); Spinobulbar Muscular Atrophy (SBMA); Spinocerebellar Ataxias (SCA types 1, 2, 3, 6, 7, and 17); and Dentatorubro-Pallidoluysian Atrophy (DRPLA). Other trinucleotide repeat expansion disorders either do not involve the CAG triplet, or the CAG triplet is not in the coding region of the gene and are referred to as the non-polyglutamine disorders. Non-polyglutamine disorders may include Fragile X Syndrome (FRAXA); Fragile XE Mental Retardation (FRAXE); Friedreich Ataxia (FRDA); Myotonic Dystrophy (DM); and Spinocerebellar Ataxias (SCA types 8, and 12).

In one embodiment, a method of the invention may be used to create a genetically modified animal or cell in which at least one chromosomal sequence associated with a trinucleotide repeat disorder has been edited. Suitable chromosomal edits may include, but are not limited to, the type of edits detailed in section I(f) above.

In each of the above embodiments, one or more chromosomal sequences associated with a trinucleotide repeat disorder may be edited. A trinucleotide repeat disorder associated protein or control sequence may typically be selected based on an experimental association of the protein or sequence to a trinucleotide repeat expansion disorder. Trinucleotide repeat expansion proteins may include proteins associated with susceptibility for developing a trinucleotide repeat expansion disorder, the presence of a trinucleotide repeat expansion disorder, the severity of a trinucleotide repeat expansion disorder or any combination thereof. For example, the production rate or circulating concentration of a protein associated with a trinucleotide repeat expansion disorder may be elevated or depressed in a population having a trinucleotide repeat expansion disorder relative to a population lacking the trinucleotide repeat expansion disorder. Differences in protein levels may be assessed using proteomic or genomic analysis techniques known in the art.

Non-limiting examples of proteins associated with trinucleotide repeat expansion disorders include AR (androgen receptor), FMR1 (fragile X mental retardation 1), HTT (huntingtin), DMPK (dystrophia myotonica-protein kinase), FXN (frataxin), ATXN2 (ataxin 2), ATN1 (atrophin 1), FEN1 (flap structure-specific endonuclease 1), TNRC6A (trinucleotide repeat containing 6A), PABPN1 (poly(A) binding protein, nuclear 1), JPH3 (junctophilin 3), MED15 (mediator complex subunit 15), ATXN1 (ataxin 1), ATXN3 (ataxin 3), TBP (TATA box binding protein), CACNA1A (calcium channel, voltage-dependent, P/Q type, alpha 1A subunit), ATXN80S (ATXN8 opposite strand (non-protein coding)), PPP2R2B (protein phosphatase 2, regulatory subunit B, beta), ATXN7 (ataxin 7), TNRC6B (trinucleotide repeat containing 6B), TNRC6C (trinucleotide repeat containing 6C), CELF3 (CUGBP, Elav-like family member 3), MAB21L1 (mab-21-like 1 (C. elegans)), MSH2 (mutS homolog 2, colon cancer, nonpolyposis type 1 (E. coli)), TMEM185A (transmembrane protein 185A), SIX5 (SIX homeobox 5), CNPY3 (canopy 3 homolog (zebrafish)), FRAXE (fragile site, folic acid type, rare, fra(X)(q28) E), GNB2 (guanine nucleotide binding protein (G protein), beta polypeptide 2), RPL14 (ribosomal protein L14), ATXN8 (ataxin 8), INSR (insulin receptor), TTR (transthyretin), EP400 (E1A binding protein p400), GIGYF2 (GRB10 interacting GYF protein 2), OGG1 (8-oxoguanine DNA glycosylase), STC1 (stanniocalcin 1), CNDP1 (carnosine dipeptidase 1 (metallopeptidase M20 family)), C10orf2 (chromosome 10 open reading frame 2), MAML3 mastermind-like 3 (Drosophila), DKC1 (dyskeratosis congenita 1, dyskerin), PAXIP1 (PAX interacting (with transcription-activation domain) protein 1), CASK (calcium/calmodulin-dependent serine protein kinase (MAGUK family)), MAPT (microtubule-associated protein tau), SP1 (Sp1 transcription factor), POLG (polymerase (DNA directed), gamma), AFF2 (AF4/FMR2 family, member 2), THBS1 (thrombospondin 1), TP53 (tumor protein p53), ESR1 (estrogen receptor 1), CGGBP1 (CGG triplet repeat binding protein 1), ABT1 (activator of basal transcription 1), KLK3 (kallikrein-related peptidase 3), PRNP (prion protein), JUN (jun oncogene), KCNN3 (potassium intermediate/small conductance calcium-activated channel, subfamily N, member 3), BAX (BCL2-associated X protein), FRAXA (fragile site, folic acid type, rare, fra(X)(q27.3) A (macroorchidism, mental retardation)), KBTBD10 (kelch repeat and BTB (POZ) domain containing 10), MBNL1 (muscleblind-like (Drosophila)), RAD51 (RAD51 homolog (RecA homolog, E. coli) (S. cerevisiae)), NCOA3 (nuclear receptor coactivator 3), ERDA1 (expanded repeat domain, CAG/CTG 1), TSC1 (tuberous sclerosis 1), COMP (cartilage oligomeric matrix protein), GCLC (glutamate-cysteine ligase, catalytic subunit), RRAD (Ras-related associated with diabetes), MSH3 (mutS homolog 3 (E. coli)), DRD2 (dopamine receptor D2), CD44 (CD44 molecule (Indian blood group)), CTCF (CCCTC-binding factor (zinc finger protein)), CCND1 (cyclin D1), CLSPN (claspin homolog (Xenopus laevis)), MEF2A (myocyte enhancer factor 2A), PTPRU (protein tyrosine phosphatase, receptor type, U), GAPDH (glyceraldehyde-3-phosphate dehydrogenase), TRIM22 (tripartite motif-containing 22), WT1 (Wilms tumor 1), AHR (aryl hydrocarbon receptor), GPX1 (glutathione peroxidase 1), TPMT (thiopurine S-methyltransferase), NDP (Norrie disease (pseudoglioma)), ARX (aristaless related homeobox), MUS81 (MUS81 endonuclease homolog (S. cerevisiae)), TYR (tyrosinase (oculocutaneous albinism IA)), EGR1 (early growth response 1), UNG (uracil-DNA glycosylase), NUMBL (numb homolog (Drosophila)-like), FABP2 (fatty acid binding protein 2, intestinal), EN2 (engrailed homeobox 2), CRYGC (crystallin, gamma C), SRP14 (signal recognition particle 14 kDa (homologous Alu RNA binding protein)), CRYGB (crystallin, gamma B), PDCD1 (programmed cell death 1), HOXA1 (homeobox A1), ATXN2L (ataxin 2-like), PMS2 (PMS2 postmeiotic segregation increased 2 (S. cerevisiae)), GLA (galactosidase, alpha), CBL (Cas-Br-M (murine) ecotropic retroviral transforming sequence), FTH1 (ferritin, heavy polypeptide 1), IL12RB2 (interleukin 12 receptor, beta 2), OTX2 (orthodenticle homeobox 2), HOXA5 (homeobox A5), POLG2 (polymerase (DNA directed), gamma 2, accessory subunit), DLX2 (distal-less homeobox 2), SIRPA (signal-regulatory protein alpha), OTX1 (orthodenticle homeobox 1), AHRR (aryl-hydrocarbon receptor repressor), MANF (mesencephalic astrocyte-derived neurotrophic factor), TMEM158 (transmembrane protein 158 (gene/pseudogene)), and ENSG00000078687.

Exemplary proteins associated with trinucleotide repeat expansion disorders include HTT (Huntingtin), AR (androgen receptor), FXN (frataxin), Atxn3 (ataxin), Atxn1 (ataxin), Atxn2 (ataxin), Atxn7 (ataxin), Atxn10 (ataxin), DMPK (dystrophia myotonica-protein kinase), Atn1 (atrophin 1), CBP (creb binding protein), VLDLR (very low density lipoprotein receptor), and any combination thereof.

In certain embodiments, an animal created by a method of the invention may be used to study the effects of mutations on the animal and the development and/or progression of a trinucleotide repeat disorder using measures commonly used in the study of a trinucleotide repeat disorder.

### O. Neurotransmission Disorders

Non-limiting examples of a neurotransmission disorder include amylotropic lateral sclerosis (ALS), spinocerebellar ataxias (SCA) including SCA2, Alzheimer's; autism, mental retardation, Rett's syndrome, fragile X syndrome, depression, schizophrenia, bi-polar disorders, disorders of learning, memory or behavior, anxiety, brain injury, seizure disorders, Huntington's disease (chorea), mania, neuroleptic malignant syndrome, pain, Parkinsonism, Parkinson's disease, tardive dyskinesia, myasthenia gravis, episodic ataxias, hyperkalemic periodic paralysis, hypokalemic periodic paralysis, Lambert-Eaton syndrome, paramyotonia congenita, Rasmussen's encephalitis, startle disease (hyperexplexia, stiff baby syndrome), and the effects of poisoning such as botulism, mushroom poisoning, organophosphates, snake venom such as from Bungarus multicinctus (Taiwanese banded krait). In one embodiment, a method of the invention may be used to create an animal or cell in which at least one chromosomal sequence associated with a neurotransmission disorder has been edited. Suitable chromosomal edits may include, but are not limited to, the type of edits detailed in section I(f) above.

In each of the above embodiments, one or more chromosomal sequences associated with a neurotransmission disorder may be edited. A neurotransmission disorder associated protein or control sequence may typically be selected based on an experimental association of the protein to a neurotransmission disorder. Neurotransmission disorder-related proteins include proteins associated with the susceptibility for developing a neurotransmission disorder, the presence of a neurotransmission disorder, the severity of a neurotransmission disorder or any combination thereof. For example, the production rate or circulating concentration of a neurotransmission disorder-related protein may be elevated or depressed in a population having a neurotransmission disorder relative to a population lacking the neurotransmission disorder. Differences in protein levels may be assessed using proteomic or genomic analysis techniques known in the art.

Non-limiting examples of neurotransmission disorder-related proteins include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), HTR2c (5-hydroxytryptamine (serotonin) receptor 2C), SLC1A2 (solute carrier family 1 (glial high affinity glutamate transporter), member 2), GRM5 (glutamate receptor, metabotropic 5), GRM2 (glutamate receptor, metabotropic 2), GABRG3 (gamma-aminobutyric acid (GABA) A receptor, gamma 3), CACNA1B (calcium channel, voltage-dependent, N type, alpha 1B subunit), NOS2 (nitric oxide synthase 2, inducible), SLC6A5 (solute carrier family 6 (neurotransmitter transporter, glycine), member 5), GABRG1 (gamma-aminobutyric acid (GABA) A receptor, gamma 1), NOS3 (nitric oxide synthase 3 (endothelial cell)), GRM3 (glutamate receptor, metabotropic 3), HTR6 (5-hydroxytryptamine (serotonin) receptor 6), SLC1A3 (solute carrier family 1 (glial high affinity glutamate transporter), member 3), GRM7 (glutamate receptor, metabotropic 7), HRH1 (histamine receptor H1), SLC1A1 (solute carrier family 1 (neuronal/epithelial high affinity glutamate transporter, system Xag), member 1), GRM4 (glutamate receptor, metabotropic 4), GLUD2 (glutamate dehydrogenase 2), ADRA2B (adrenergic, alpha-2B-, receptor), SLC1A6 (solute carrier family 1 (high affinity aspartate/glutamate transporter), member 6), GRM6 (glutamate receptor, metabotropic 6), SLC1A7 (solute carrier family 1 (glutamate transporter), member 7), SLC6A11 (solute carrier family 6 (neurotransmitter transporter, GABA), member 11), CACNA1A (calcium channel, voltage-dependent, P/Q type, alpha 1A subunit), CACNA1G (calcium channel, voltage-dependent, T type, alpha 1G subunit), GRM1 (glutamate receptor, metabotropic 1), CACNA1H (calcium channel, voltage-dependent, T type, alpha 1H subunit), GRM8 (glutamate receptor, metabotropic 8), CHRNA3 (cholinergic receptor, nicotinic, alpha 3), P2RY2 (purinergic receptor P2Y, G-protein coupled, 2), TRPV6 (transient receptor potential cation channel, subfamily V, member 6), CACNA1E (calcium channel, voltage-dependent, R type, alpha 1E subunit), ACCN1 (amiloride-sensitive cation channel 1, neuronal), CACNA1I (calcium channel, voltage-dependent, T type, alpha 1I subunit), GABARAP (GABA (A) receptor-associated protein), P2RY1 (purinergic receptor P2Y, G-protein coupled, 1), P2RY6 (pyrimidinergic receptor P2Y, G-protein coupled, 6), RPH3A (rabphilin 3A homolog (mouse)), HDC (histidine decarboxylase), P2RY14 (purinergic receptor P2Y, G-protein coupled, 14), P2RY4 (pyrimidinergic receptor P2Y, G-protein coupled, 4), P2RY10 (purinergic receptor P2Y, G-protein coupled, 10), SLC28A3 (solute carrier family 28 (sodium-coupled nucleoside transporter), member 3), NOSTRIN (nitric oxide synthase trafficker), P2RY13 (purinergic receptor P2Y, G-protein coupled, 13), P2RY8 (purinergic receptor P2Y, G-protein coupled, 8), P2RY11 (purinergic receptor P2Y, G-protein coupled, 11), SLC6A3 (solute carrier family 6 (neurotransmitter transporter, dopamine), member 3), HTR3A (5-hydroxytryptamine (serotonin) receptor 3A), DRD2 (dopamine receptor D2), HTR2A (5-hydroxytryptamine (serotonin) receptor 2A), TH (tyrosine hydroxylase), CNR1 (cannabinoid receptor 1 (brain)), VIP (vasoactive intestinal peptide), NPY (neuropeptide Y), GAL (galanin prepropeptide), TAC1 (tachykinin, precursor 1), SYP (synaptophysin), SLC6A4 (solute carrier family 6 (neurotransmitter transporter, serotonin), member 4), DBH (dopamine beta-hydroxylase (dopamine beta-monooxygenase)), DRD3 (dopamine receptor D3), NR3C1 (nuclear receptor subfamily 3, group C, member 1 (glucocorticoid receptor)), HTR1B (5-hydroxytryptamine (serotonin) receptor 1B), GABBR1 (gamma-aminobutyric acid (GABA) B receptor, 1), CALCA (calcitonin-related polypeptide alpha), CRH (corticotropin releasing hormone), HTR1A (5-hydroxytryptamine (serotonin) receptor 1A), TACR2 (tachykinin receptor 2), COMT (catechol-O-methyltransferase), GRIN2B (glutamate receptor, ionotropic, N-methyl D-aspartate 2B), GRIN2A (glutamate receptor, ionotropic, N-methyl D-aspartate 2A), PRL (prolactin), ACHE (acetylcholinesterase (Yt blood group)), ADRB2 (adrenergic, beta-2-, receptor, surface), ACE (angiotensin I converting enzyme (peptidyl-dipeptidase A) 1), SNAP25 (synaptosomal-associated protein, 25 kDa), GABRA5 (gamma-aminobutyric acid (GABA) A receptor, alpha 5), MECP2 (methyl CpG binding protein 2 (Rett syndrome)), BCHE (butyrylcholinesterase), ADRB1 (adrenergic, beta-1-, receptor), GABRA1 (gamma-aminobutyric acid (GABA) A receptor, alpha 1), GCH1 (GTP cyclohydrolase 1), DDC (dopa decarboxylase (aromatic L-amino acid decarboxylase)), MAOB (monoamine oxidase B), DRD5 (dopamine receptor D5), GABRE (gamma-aminobutyric acid (GABA) A receptor, epsilon), SLC6A2 (solute carrier family 6 (neurotransmitter transporter, noradrenalin), member 2), GABRR2 (gamma-aminobutyric acid (GABA) receptor, rho 2), SV2A (synaptic vesicle glycoprotein 2A), GABRR1 (gamma-aminobutyric acid (GABA) receptor, rho 1), GHRH (growth hormone releasing hormone), CCK (cholecystokinin), PDYN (prodynorphin), SLC6A9 (solute carrier family 6 (neurotransmitter transporter, glycine), member 9), KCND1 (potassium voltage-gated channel, Shal-related subfamily, member 1), SRR (serine racemase), DYT10 (dystonia 10), MAPT (microtubule-associated protein tau), APP (amyloid beta (A4) precursor protein), CTSB (cathepsin B), ADA (adenosine deaminase), AKT1 (v-akt murine thymoma viral oncogene homolog 1), GRIN1 (glutamate receptor, ionotropic, N-methyl D-aspartate 1), BDNF (brain-derived neurotrophic factor), HMOX1 (heme oxygenase (decycling) 1), OPRM1 (opioid receptor, mu 1), GRIN2C (glutamate receptor, ionotropic, N-methyl D-aspartate 2C), GRIA1 (glutamate receptor, ionotropic, AMPA 1), GABRA6 (gamma-aminobutyric acid (GABA) A receptor, alpha 6), FOS (FBJ murine osteosarcoma viral oncogene homolog), GABRG2 (gamma-aminobutyric acid (GABA) A receptor, gamma 2), GABRB3 (gamma-aminobutyric acid (GABA) A receptor, beta 3), OPRK1 (opioid receptor, kappa 1), GABRB2 (gamma-aminobutyric acid (GABA) A receptor, beta 2), GABRD (gamma-aminobutyric acid (GABA) A receptor, delta), ALDH5A1 (aldehyde dehydrogenase 5 family, member A1), GAD1 (glutamate decarboxylase 1 (brain, 67 kDa)), NSF (N-ethylmaleimide-sensitive factor), GRIN2D (glutamate receptor, ionotropic, N-methyl D-aspartate 2D), ADORA1 (adenosine A1 receptor), GABRA2 (gamma-aminobutyric acid (GABA) A receptor, alpha 2), GLRA1 (glycine receptor, alpha 1), CHRM3 (cholinergic receptor, muscarinic 3), CHAT (choline acetyltransferase), KNG1 (kininogen 1), HMOX2 (heme oxygenase (decycling) 2), DRD4 (dopamine receptor D4), MAOA (monoamine oxidase A), CHRM2 (cholinergic receptor, muscarinic 2), ADORA2A (adenosine A2a receptor), STXBP1 (syntaxin binding protein 1), GABRA3 (gamma-aminobutyric acid (GABA) A receptor, alpha 3), TPH1 (tryptophan hydroxylase 1), HCRTR1 (hypocretin (orexin) receptor 1), HCRTR2 (hypocretin (orexin) receptor 2), CHRM1 (cholinergic receptor, muscarinic 1), FOLH1 (folate hydrolase (prostate-specific membrane antigen) 1), AANAT (arylalkylamine N-acetyltransferase), INS (insulin), NR3C2 (nuclear receptor subfamily 3, group C, member 2), FAAH (fatty acid amide hydrolase), GALR2 (galanin receptor 2), ADCYAP1 (adenylate cyclase activating polypeptide 1 (pituitary)), PPP1R1B (protein phosphatase 1, regulatory (inhibitor) subunit 1B), HOMER1 (homer homolog 1 (Drosophila)), ADCY10 (adenylate cyclase 10 (soluble)), PSEN2 (presenilin 2 (Alzheimer disease 4)), UBE3A (ubiquitin protein ligase E3A), SOD1 (superoxide dismutase 1, soluble), LYN (v-yes-1 Yamaguchi sarcoma viral related oncogene homolog), TSC2 (tuberous sclerosis 2), PRKCA (protein kinase C, alpha), PPARG (peroxisome proliferator-activated receptor gamma), ESR1 (estrogen receptor 1), NTRK1 (neurotrophic tyrosine kinase, receptor, type 1), EGFR (epidermal growth factor receptor (erythroblastic leukemia viral (v-erb-b) oncogene homolog, avian)), S100B (S100 calcium binding protein B), NTRK3 (neurotrophic tyrosine kinase, receptor, type 3), PLCG2 (phospholipase C, gamma 2 (phosphatidylinositol-specific)), NTRK2 (neurotrophic tyrosine kinase, receptor, type 2), DNMT1 (DNA (cytosine-5-)-methyltransferase 1), EGF (epidermal growth factor (beta-urogastrone)), GRIA3 (glutamate receptor, ionotrophic, AMPA 3), NCAM1 (neural cell adhesion molecule 1), CDKN1A (cyclin-dependent kinase inhibitor 1A (p21, Cip1)), BCL2L1 (BCL2-like 1), TP53 (tumor protein p53), CASP9 (caspase 9, apoptosis-related cysteine peptidase), CCKBR (cholecystokinin B receptor), PARK2 (Parkinson's disease (autosomal recessive, juvenile) 2, parkin), ADRA1B (adrenergic, alpha-1B-, receptor), CASP3 (caspase 3, apoptosis-related cysteine peptidase), PRNP (prion protein), CRHR1 (corticotropin releasing hormone receptor 1), L1CAM (L1 cell adhesion molecule), NGFR (nerve growth factor receptor (TNFR superfamily, member 16)), CREB1 (cAMP responsive element binding protein 1), PLCG1 (phospholipase C, gamma 1), CAV1 (caveolin 1, caveolae protein, 22 kDa), ABCC8 (ATP-binding cassette, sub-family C(CFTR/MRP), member 8), ACTN2 (actinin, alpha 2), GRIA2 (glutamate receptor, ionotropic, AMPA 2), HPRT1 (hypoxanthine phosphoribosyltransferase 1), SYN1 (synapsin I), CSNK2A1 (casein kinase 2, alpha 1 polypeptide), GRIK1 (glutamate receptor, ionotropic, kainate 1), ABCB1 (ATP-binding cassette, sub-family B (MDR/TAP), member 1), AVPR2 (arginine vasopressin receptor 2), HTR4 (5-hydroxytryptamine (serotonin) receptor 4), C3 (complement component 3), AGT (angiotensinogen (serpin peptidase inhibitor, clade A, member 8)), AGTR1 (angiotensin II receptor, type 1), CDK5 (cyclin-dependent kinase 5), LRP1 (low density lipoprotein receptor-related protein 1), ARRB2 (arrestin, beta 2), PLD2 (phospholipase D2), OPRD1 (opioid receptor, delta 1), GNB3 (guanine nucleotide binding protein (G protein), beta polypeptide 3), PIK3CG (phosphoinositide-3-kinase, catalytic, gamma polypeptide), APAF1 (apoptotic peptidase activating factor 1), SSTR2 (somatostatin receptor 2), IL2 (interleukin 2), ADORA3 (adenosine A3 receptor), ADRA1A (adrenergic, alpha-1A-, receptor), HTR7 (5-hydroxytryptamine (serotonin) receptor 7 (adenylate cyclase-coupled)), ADRBK2 (adrenergic, beta, receptor kinase 2), ALOX5 (arachidonate 5-lipoxygenase), NPR1 (natriuretic peptide receptor A/guanylate cyclase A (atrionatriuretic peptide receptor A)), AVPR1A (arginine vasopressin receptor 1A), CHRNB1 (cholinergic receptor, nicotinic, beta 1 (muscle)), SET (SET nuclear oncogene), PAH (phenylalanine hydroxylase), POMC (proopiomelanocortin), LEPR (leptin receptor), SDC2 (syndecan 2), VIPR1 (vasoactive intestinal peptide receptor 1), DBI (diazepam binding inhibitor (GABA receptor modulator, acyl-Coenzyme A binding protein)), NPY1R (neuropeptide Y receptor Y1), NPR2 (natriuretic peptide receptor B/guanylate cyclase B (atrionatriuretic peptide receptor B)), CNR2 (cannabinoid receptor 2 (macrophage)), LEP (leptin), CCKAR (cholecystokinin A receptor), GLRB (glycine receptor, beta), KCNQ2 (potassium voltage-gated channel, KQT-like subfamily, member 2), CHRNA2 (cholinergic receptor, nicotinic, alpha 2 (neuronal)), BDKRB2 (bradykinin receptor B2), CHRNA1 (cholinergic receptor, nicotinic, alpha 1 (muscle)), CHRND (cholinergic receptor, nicotinic, delta), CHRNA7 (cholinergic receptor, nicotinic, alpha 7), PLD1 (phospholipase D1, phosphatidylcholine-specific), NRXN1 (neurexin 1), NRP1 (neuropilin 1), DLG3 (discs, large homolog 3 (Drosophila)), GNAQ (guanine nucleotide binding protein (G protein), q polypeptide), DRD1 (dopamine receptor D1), PRKG1 (protein kinase, cGMP-dependent, type I), CNTNAP2 (contactin associated protein-like 2), EDN3 (endothelin 3), ABAT (4-aminobutyrate aminotransferase), TDO2 (tryptophan 2,3-dioxygenase), NEUROD1 (neurogenic differentiation 1), CHRNE (cholinergic receptor, nicotinic, epsilon), CHRNB2 (cholinergic receptor, nicotinic, beta 2 (neuronal)), CHRNB3 (cholinergic receptor, nicotinic, beta 3), HTR1D (5-hydroxytryptamine (serotonin) receptor 1D), ADRA1D (adrenergic, alpha-1D-, receptor), HTR2B (5-hydroxytryptamine (serotonin) receptor 2B), GRIK3 (glutamate receptor, ionotropic, kainate 3), NPY2R (neuropeptide Y receptor Y2), GRIK5 (glutamate receptor, ionotropic, kainate 5), GRIA4 (glutamate receptor, ionotrophic, AMPA 4), EDN1 (endothelin 1), PRLR (prolactin receptor), GABRB1 (gamma-aminobutyric acid (GABA) A receptor, beta 1), GARS (glycyl-tRNA synthetase), GRIK2 (glutamate receptor, ionotropic, kainate 2), ALOX12 (arachidonate 12-lipoxygenase), GAD2 (glutamate decarboxylase 2 (pancreatic islets and brain, 65 kDa)), LHCGR (luteinizing hormone/choriogonadotropin receptor), SHMT1 (serine hydroxymethyltransferase 1 (soluble)), PDXK (pyridoxal (pyridoxine, vitamin B6) kinase), LIF (leukemia inhibitory factor (cholinergic differentiation factor)), PLCD1 (phospholipase C, delta 1), NTF3 (neurotrophin 3), NFE2L2 (nuclear factor (erythroid-derived 2)-like 2), PLCB4 (phospholipase C, beta 4), GNRHR (gonadotropin-releasing hormone receptor), NLGN1 (neuroligin 1), PPP2R4 (protein phosphatase 2A activator, regulatory subunit 4), SSTR3 (somatostatin receptor 3), CRHR2 (corticotropin releasing hormone receptor 2), NGF (nerve growth factor (beta polypeptide)), NRCAM (neuronal cell adhesion molecule), NRXN3 (neurexin 3), GNRH1 (gonadotropin-releasing hormone 1 (luteinizing-releasing hormone)), TRHR (thyrotropin-releasing hormone receptor), ARRB1 (arrestin, beta 1), INPP1 (inositol polyphosphate-1-phosphatase), PTN (pleiotrophin), PSMD10 (proteasome (prosome, macropain) 26S subunit, non-ATPase, 10), DLG1 (discs, large homolog 1 (Drosophila)), PSMB8 (proteasome (prosome, macropain) subunit, beta type, 8 (large multifunctional peptidase 7)), CYCS (cytochrome c, somatic), ADORA2B (adenosine A2b receptor), ADRB3 (adrenergic, beta-3-, receptor), CHGA (chromogranin A (parathyroid secretory protein 1)), ADM (adrenomedullin), GABRP (gamma-aminobutyric acid (GABA) A receptor, pi), GLRA2 (glycine receptor, alpha 2), PRKG2 (protein kinase, cGMP-dependent, type II), GLS (glutaminase), TACR3 (tachykinin receptor 3), ALDH7A1 (aldehyde dehydrogenase 7 family, member A1), GABBR2 (gamma-aminobutyric acid (GABA) B receptor, 2), GDNF (glial cell derived neurotrophic factor), CNTFR (ciliary neurotrophic factor receptor), CNTN2 (contactin 2 (axonal)), TOR1A (torsin family 1, member A (torsin A)), CNTN1 (contactin 1), CAMK1 (calcium/calmodulin-dependent protein kinase I), NPPB (natriuretic peptide precursor B), OXTR (oxytocin receptor), OSM (oncostatin M), VIPR2 (vasoactive intestinal peptide receptor 2), CHRNB4 (cholinergic receptor, nicotinic, beta 4), CHRNA5 (cholinergic receptor, nicotinic, alpha 5), AVP (arginine vasopressin), RELN (reelin), GRLF1 (glucocorticoid receptor DNA binding factor 1), NPR3 (natriuretic peptide receptor C/guanylate cyclase C (atrionatriuretic peptide receptor C)), GRIK4 (glutamate receptor, ionotropic, kainate 4), KISS1 (KiSS-1 metastasis-suppressor), HTR5A (5-hydroxytryptamine (serotonin) receptor 5A), ADCYAP1R1 (adenylate cyclase activating polypeptide 1 (pituitary) receptor type I), GABRA4 (gamma-aminobutyric acid (GABA) A receptor, alpha 4), GLRA3 (glycine receptor, alpha 3), INHBA (inhibin, beta A), DLG2 (discs, large homolog 2 (Drosophila)), PPYR1 (pancreatic polypeptide receptor 1), SSTR4 (somatostatin receptor 4), NPPA (natriuretic peptide precursor A), SNAP23 (synaptosomal-associated protein, 23 kDa), AKAP9 (A kinase (PRKA) anchor protein (yotiao) 9), NRXN2 (neurexin 2), FHL2 (four and a half LIM domains 2), TJP1 (tight junction protein 1 (zona occludens 1)), NRG1 (neuregulin 1), CAMK4 (calcium/calmodulin-dependent protein kinase IV), CAV3 (caveolin 3), VAMP2 (vesicle-associated membrane protein 2 (synaptobrevin 2)), GALR1 (galanin receptor 1), GHRHR (growth hormone releasing hormone receptor), HTR1E (5-hydroxytryptamine (serotonin) receptor 1E), PENK (proenkephalin), HTT (huntingtin), HOXA1 (homeobox A1), NPY5R (neuropeptide Y receptor Y5), UNC119 (unc-119 homolog (C. elegans)), TAT (tyrosine aminotransferase), CNTF (ciliary neurotrophic factor), SHMT2 (serine hydroxymethyltransferase 2 (mitochondrial)), ENTPD1 (ectonucleoside triphosphate diphosphohydrolase 1), GRIP1 (glutamate receptor interacting protein 1), GRP (gastrin-releasing peptide), NCAM2 (neural cell adhesion molecule 2), SSTR1 (somatostatin receptor 1), CLTB (clathrin, light chain (Lcb)), DAO (D-amino-acid oxidase), QDPR (quinoid dihydropteridine reductase), PYY (peptide YY), PNMT (phenylethanolamine N-methyltransferase), NTSR1 (neurotensin receptor 1 (high affinity)), NTS (neurotensin), HCRT (hypocretin (orexin) neuropeptide precursor), SNAP29 (synaptosomal-associated protein, 29 kDa), SNAP91 (synaptosomal-associated protein, 91 kDa homolog (mouse)), MADD (MAP-kinase activating death domain), IDO1 (indoleamine 2,3-dioxygenase 1), TPH2 (tryptophan hydroxylase 2), TAC3 (tachykinin 3), GRIN3A (glutamate receptor, ionotropic, N-methyl-D-aspartate 3A), REN (renin), GALR3 (galanin receptor 3), MAGI2 (membrane associated guanylate kinase, WW and PDZ domain containing 2), KCNJ9 (potassium inwardly-rectifying channel, subfamily J, member 9), BDKRB1 (bradykinin receptor B1), CHRNA6 (cholinergic receptor, nicotinic, alpha 6), CHRM5 (cholinergic receptor, muscarinic 5), CHRNG (cholinergic receptor, nicotinic, gamma), SLC6A1 (solute carrier family 6 (neurotransmitter transporter, GABA), member 1), ENTPD2 (ectonucleoside triphosphate diphosphohydrolase 2), CALCB (calcitonin-related polypeptide beta), SHBG (sex hormone-binding globulin), SERPINA6 (serpin peptidase inhibitor, clade A (alpha-1 antiproteinase, antitrypsin), member 6), NRG2 (neuregulin 2), PNOC (prepronociceptin), NAPA (N-ethylmaleimide-sensitive factor attachment protein, alpha), PICK1 (protein interacting with PRKCA 1), PLCD4 (phospholipase C, delta 4), GCDH (glutaryl-Coenzyme A dehydrogenase), NLGN2 (neuroligin 2), NBEA (neurobeachin), ATP10A (ATPase, class V, type 10A), RAPGEF4 (Rap guanine nucleotide exchange factor (GEF) 4), UCN (urocortin), PCSK6 (proprotein convertase subtilisin/kexin type 6), HTR1F (5-hydroxytryptamine (serotonin) receptor 1F), SGCB (sarcoglycan, beta (43 kDa dystrophin-associated glycoprotein)), GABRQ (gamma-aminobutyric acid (GABA) receptor, theta), GHRL (ghrelin/obestatin prepropeptide), NCALD (neurocalcin delta), NEUROD2 (neurogenic differentiation 2), DPEP1 (dipeptidase 1 (renal)), SLC1A4 (solute carrier family 1 (glutamate/neutral amino acid transporter), member 4), DNM3 (dynamin 3), SLC6A12 (solute carrier family 6 (neurotransmitter transporter, betaine/GABA), member 12), SLC6A6 (solute carrier family 6 (neurotransmitter transporter, taurine), member 6), YME1L1 (YME1-like 1 (S. cerevisiae)), VSNL1 (visinin-like 1), SLC17A7 (solute carrier family 17 (sodium-dependent inorganic phosphate cotransporter), member 7), HOMER2 (homer homolog 2 (Drosophila)), SYT7 (synaptotagmin VII), TFIP11 (tuftelin interacting protein 11), GMFB (glia maturation factor, beta), PREB (prolactin regulatory element binding), NTSR2 (neurotensin receptor 2), NTF4 (neurotrophin 4), PPP1R9B (protein phosphatase 1, regulatory (inhibitor) subunit 9B), DISC1 (disrupted in schizophrenia 1), NRG3 (neuregulin 3), OXT (oxytocin, prepropeptide), TRH (thyrotropin-releasing hormone), NISCH (nischarin), CRHBP (corticotropin releasing hormone binding protein), SLC6A13 (solute carrier family 6 (neurotransmitter transporter, GABA), member 13), NPPC (natriuretic peptide precursor C), CNTN3 (contactin 3 (plasmacytoma associated)), KAT5 (K (lysine) acetyltransferase 5), CNTN6 (contactin 6), KIAA0101 (KIAA0101), PANX1 (pannexin 1), CTSL1 (cathepsin L1), EARS2 (glutamyl-tRNA synthetase 2, mitochondrial (putative)), CRIPT (cysteine-rich PDZ-binding protein), CORT (cortistatin), DLGAP4 (discs, large (Drosophila) homolog-associated protein 4), ASTN2 (astrotactin 2), HTR3B (5-hydroxytryptamine (serotonin) receptor 3B), PMCH (pro-melanin-concentrating hormone), TSPO (translocator protein (18 kDa)), GDF2 (growth differentiation factor 2), CNTNAP1 (contactin associated protein 1), GNRH2 (gonadotropin-releasing hormone 2), AUTS2 (autism susceptibility candidate 2), SV2C (synaptic vesicle glycoprotein 2C), CARTPT (CART prepropeptide), NSUN4 (NOP2/Sun domain family, member 4), CNTN5 (contactin 5), NEUROD4 (neurogenic differentiation 4), NEUROG1 (neurogenin 1), SLTM (SAFB-like, transcription modulator), GNRHR2 (gonadotropin-releasing hormone (type 2) receptor 2), ASTN1 (astrotactin 1), SLC22A18 (solute carrier family 22, member 18), SLC17A6 (solute carrier family 17 (sodium-dependent inorganic phosphate cotransporter), member 6), GABRR3 (gamma-aminobutyric acid (GABA) receptor, rho 3), DAOA (D-amino acid oxidase activator), ENSG00000123384, nd NOS2P1 (nitric oxide synthase 2 pseudogene 1).

Exemplary neurotransmission-related proteins include 5-HTT (5-hydroxyltryptamine transporter), SLC6A4 (Solute carrier family 6, member 4), COMT (Catechol-O-methyltransferase), DRD1A (Dopamine receptor D1A), SLC6A3 (Solute carrier family 6, member 3), DAO1 (D-amino-acid oxidase), DTNBP1 (Dystrobrevin binding protein 1), and any combination thereof.

In certain embodiments, an animal created by a method of the invention may be used to study the effects of mutations on the animal and on the development and/or progression of a neurotransmission disorder using measures commoningly used in the study of a neurotransmission disorder.

ii. Pharmacological Models

A method of the invention may be used to create an animal or cell that may be used as a pharmacological model. Such a pharmacological model may be a model for pharmacokinetics or a model for pharmacodynamics. For instance, in one embodiment, a method of the invention may be used to create an animal or cell that comprises a chromosomal edit in one or more nucleic acid sequences associated with the metabolism of a pharmaceutically active compound. Such an animal or cell may be used to study the effect of the nucleic acid sequence on the pharmaceutical compound.

Alternatively, a method of the invention may be used to create an animal or cell that comprises a chromosomal edit in a disease associated sequence. Such an animal or cell may be used for assessing the effect(s) of a therapeutic agent in the development or progression of the disease. For example, the effect(s) of a therapeutic agent may be measured in a “humanized” animal, such that the information gained therefrom may be used to predict the effect of the agent in a human. In general, the method comprises contacting a genetically modified animal comprising at least one edited chromosomal sequence encoding a protein associated with the disease with the therapeutic agent, and comparing results of a selected parameter to results obtained from contacting a wild-type animal with the same agent. Non-limiting examples of suitable diseases include those listed in section II(a)i.

Also provided are methods to assess the effect(s) of an agent in an isolated cell comprising at least one edited chromosomal sequence encoding a protein associated with a disease, as well as methods of using lysates of such cells (or cells derived from a genetically modified animal disclosed herein) to assess the effect(s) of an agent. For example, the role of a particular protein associated with a disease in the metabolism of a particular agent may be determined using such methods. Similarly, substrate specificity and pharmacokinetic parameters may be readily determined using such methods. Those of skill in the art are familiar with suitable tests and/or procedures.

In one embodiment, a method of the invention may be used to create an animal or cell in which at least one chromosomal sequence associated with toxicology has been edited. Suitable chromosomal edits may include, but are not limited to, the type of edits detailed in section I(f) above. Any chromosomal sequence or protein involved in absorption, distribution, metabolism, and excretion (ADME) and toxicology may be utilized for purposes of the present invention. The ADME and toxicology-related proteins are typically selected based on an experimental association of the protein to an ADME and toxicology-related disorder. For example, the production rate or circulating concentration of an ADME and toxicology-related protein may be elevated or depressed in a population having an ADME and toxicology disorder relative to a population lacking the ADME and toxicology disorder. Differences in protein levels may be assessed using proteomic or genomic analysis techniques known in the art.

Exemplary non-limiting examples of the chromosomal sequence or protein involved in ADME and toxicology may be chosen from Oct 1, Oct 2, Hfe2, Ppar(alpha) MDR1a (ABC Transporter ABCB1a), MDR1b (ABCB1b), BCRP (ABCC1), MRP1 (ABCG2), MRP2 (ABCC2, cMOAT), and combinations thereof.

A further aspect of the present disclosure encompasses a method for assessing the effect(s) of an agent. Suitable agents include without limit pharmaceutically active ingredients, drugs, food additives, pesticides, herbicides, toxins, industrial chemicals, household chemicals, and other environmental chemicals. For example, the effect(s) of an agent may be measured in a “humanized” genetically modified animal, such that the information gained therefrom may be used to predict the effect of the agent in a human. In general, the method comprises contacting a genetically modified animal comprising at least one inactivated chromosomal sequence involved in ADME and toxicology and at least one chromosomally integrated sequence encoding an orthologous human protein involved in ADME and toxicology with the agent, and comparing results of a selected parameter to results obtained from contacting a wild-type animal with the same agent. Selected parameters include but are not limited to (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); (g) efficacy of the agent or its metabolite(s); (h) disposition of the agent or its metabolite(s); and (i) extrahepatic contribution to metabolic rate and clearance of the agent or its metabolite(s).

Also provided are methods to assess the effect(s) of an agent in an isolated cell comprising at least one edited chromosomal sequence involved in ADME and toxicology, as well as methods of using lysates of such cells (or cells derived from a genetically modified animal disclosed herein) to assess the effect(s) of an agent. For example, the role of a particular protein involved in ADME and toxicology in the metabolism of a particular agent may be determined using such methods. Similarly, substrate specificity and pharmacokinetic parameters may be readily determined using such methods. Those of skill in the art are familiar with suitable tests and/or procedures.

Among the proteins of interest that are involved in drug ADME and toxicology are the ABC transporters, also known as efflux transport proteins. Thus, for example, the genetically modified animals as described herein containing an edited chromosomal sequences encoding an ABC transporter can be useful for screening biologically active agents including drugs and for investigating their distribution, efficacy, metabolism and/or toxicity. These screening methods are of particular use for assessing with improved predictability the behavior of a drug in a genetically modified animal as described herein, e.g. in a genetically modified rat, as a model for humans. Accordingly, the present disclosure also features a method of assessing the ADME profile of a drug in a genetically modified animal, as part of a drug screening or evaluation process. A candidate therapeutic agent, i.e, a candidate drug can be administered to a genetically modified animal that harbors a targeted gene knock-out and/or an expressed transgene, which was achieved through use of ZFNs. The knock-out or knock-in gene is associated with at least one aspect of the drug ADME profile or toxicology, and/or metabolism, and may be derived from a mouse, rat, or human genome.

For example, a method of screening for the target of a test compound can make use of a genetically modified animal in which any one or more of an ABC transporter such as Mdr1a, Mdr1b, PXR, BCRP, MRP1, or MRP2 are knocked out, thus inhibiting or eliminating transmembrane transport mediated by the knocked out protein(s). Such an animal can be exposed to a test compound suspected of inhibiting transporter activity of the knocked-out protein(s). Inhibition of transport by the compound in the genetically modified animal can be determined using any of a number of routine laboratory tests and techniques, and the inhibition of transport may be compared to that observed in a wild-type animal treated with the same test compound. A difference in the effect of the test compound in the two animals can be indicative of the target of the test compound. Further, inhibition of one or more ABC transporter proteins such as Mdr1a, Mdr1b, PXR, BCRP, MRP1, or MRP2, may improve certain ADME characteristics of a candidate therapeutic agent. For example, the absorption or efficacy of a candidate therapeutic compound may be improved by knock-ing out expression of one or more ABC transporter proteins such as Mdr1a, Mdr1b, PXR, BCRP, MRP1, or MRP2, in a particular tissue. It will thus be understood that genetically modified animals and cells as described herein, for example genetically modified animals and cells including a genetic modification of one or more ABC transporter proteins, can be used advantageously in many methods that evaluate the ADME and toxicology characteristics of a candidate therapeutic compound, to identify targets of a test compound, or to identify ways in which the ADME characteristics and toxicology of a candidate compound may be improved.

The overwhelming need to accurately predict how drugs and environmental chemicals may affect large populations can be readily appreciated. The genetically modified animals, embryos, cells and cell lines described herein can be used to analyze how various compounds may interact with biological systems. Genetically modified cells and cell lines can be used, for example, to control many of the known complexities in biological systems to improve the predictive ability of cell-based assay systems, such as those used to evaluate new molecular entities and possible drug-drug interactions. More specifically, it is recognized that biological systems typically include multiple components that respond to exposure to new, potentially harmful compounds.

The “ADMET system” has been described as including five components. The first component are those biological systems that when disrupted signal the drug metabolism system to turn on, and may include stress response and DNA repair pathways. Once the drug metabolism system is activated “xenosensors” surveil for exogenous molecules that need removal. Detection of an exogenous molecule by the xenosensors then activates a cascade of gene inductions that up-regulate the enzymes responsible for metabolizing exogenous molecules into forms for easier removal. The enzymes of the third ADMET component include Phase I enzymes that include at least three classes of oxidases, of which the best known class is the cytochrome P450 class. Cytochrome 450 enzymes typically add reactive hydroxyl moieties to potential toxins to inactivate and render the toxins more polar (soluble). The fourth component of the ADMET system includes at least seven classes of enzymes that further alter the products of Phase I enzymatic modification. Typically, these enzymes are conjugating enzymes that add hydrophilic moieties to make the now oxidized xenobiotics even more water soluble ADMET, and readily collected and excreted through urine or bile. The last component is the transporter system involving transporter proteins, such as the ABC transporters, that function as molecular pumps to facilitate the movement of the xenobiotics from one tissue to another. The transporter proteins are responsible for moving drugs into a cell, out of a cell, or through a cell.

Each component of the ADMET system has its own set of substrate structural specificities, which must be taken into account by any assay. Making predictability an even larger challenge is that, for critical members of each of the five component classes, a constellation of genetic polymorphisms exists in the population and these can dramatically affect activity towards specific xenobiotic chemical structures. The growing field of pharmacogenomics addresses the challenges created by such genetic variation. In addition, gender differences in how the different components of the xenobiotic system respond are also known to play a role in variations in drug metabolism.

Thus, genetically modified animals, cells and particularly cell lines as described herein will be useful as the basis for cell-based assays with improved predictive ability with respect to a drug's clinical outcome or a chemical's toxicological problems. Panels of cell lines are expressly contemplated for such a purpose. For example, cell-based assays can be created that are representative of the target tissue where metabolism or toxicity of a drug compound is likely to occur. Presently, standard assays are usually run in transformed cell lines that are derived from the target tissue and have some concordant functional properties. To create even better cell-based assays that are even more representative of the natural state, genetically modified and differentiated pluripotent cells could be used to replace the immortalized cell components. In other words, genetically modified cell lines can be used in more highly predictive cell-based assays suitable for high-throughput, high-content compound screening.

Accordingly, the present disclosure contemplates ZFN-mediated genetic modifications of genes relevant to each part of the xenobiotic metabolism machinery. Such modifications include knock-outs, knock-ins of reporter tags, the introduction of specific mutations known to affect activity, or combinations of these. For example, the genetically modified cells and cell lines can be used to create tissue-specific, gender-specific, and/or population-reflective transporter panels; cell-based xenosenor assay panels that are tissue-specific and functionally reflective of the population; and induction assays that measure the genetic activation of different drug metabolism components and overt toxicological responses such as genotoxicity, cardiotoxicity, and apoptosis.

According to the present disclosure, tissue-specific lines can be established that have been modified to isolate specific transporter activities and predict the reaction of populations to individual chemical entities. For example, ZFNs can be used to create transporter gene knock-outs in enterocyte cell lines, such as to introduce important, common polymorphisms into enterocyte cell lines, and in cell lines representative of liver, blood-brain-barrier (brain micro-vasculature endothelial cells), kidney and any relevant tissue-specific cell lines. Panels of cell lines can include enterocytes (Caco2 or BBe1) with knock-outs of individual transporter proteins (e.g. MDR-1, MRP1, 2, 3, 4, 6, BCRP), knock-out combinations to isolate effects of individual transporters (e.g. BCRP and MRP2, MDR-1 and MRP2, MRP-3 and MRP1), and a transporter null line (i.e. all 7 transporters knocked out). Panels of enterocytes may include knock-outs of OATP-2B1, PEPT-1, and OCT-N2. Panels of enterocytes may be created which include prevalent polymorphisms in the major transporter genes that affect drug transport and are of concern to pharmaceutical researchers.

The three xenosensors in humans (PXR, AhR and CAR) are known to have overlapping specificities in response to xenobiotics. Knowing which xenosensors are activated and to what extent by any particular chemical compound is also an important consideration for understanding drug responses, and drug-drug interactions. Creating panels of cells that report induction by the xenosensors can delineate the specificities. Further modifying the cells to address functionally important polymorphisms in the xenosensors would permit population predictions. ZFNs can be used to create knock-out cell lines analogous to transporter knockout cell lines as described above, and to create reporter cell lines that express different fluorescent proteins upon induction of different xenosensors. For example, cell lines can be created in which green FP is expressed if PXR is induced, red FP if CAR activity is induced, blue FP if AhR is induced. All lines may be constructed in the relevant tissue-type cell lines, i.e. intestine, liver, kidney, brain, and heart. Panels of cells can be created that represent the tissues most involved with drug toxicity and metabolism, and in which each xenosensor (CAR, PXR, AhR) is knocked out. Cell lines can also be produced that produce fluorescent proteins upon the activation of each of the three xenosensors.

Also contemplated are induction assays of ADME biotransformation and toxicological response genes. While the activities of each of the many Phase I and Phase II enzymes can be done today in simple biochemical assays, available assays cannot measure, in high-throughput fashion, the induction of any particular enzyme by an exogenously added xenobiotic. ZFNs can be used to create genetically modified cell lines as described herein that can provide the basis for assays that can measure the up/down regulation of key Phase I and Phase II enzymes, along with genes involved in a toxicological response. For example, ZFNs can be used to build lines that have a reporter gene (e.g. encoding fluorescent protein or luciferase) inserted proximal to the promoter of the gene being measured. These gene targets can be any of the critical Phase I, Phase II, transporter, genotox, or apoptosis/necrosis pathway components. Tissue-specific panels of cells can also be created, which report on the activation of genes encoding either the Phase I or Phase II enzymes, the transporters, or toxicity response pathways (e.g., genotoxicity or apoptosis).

iii. Developmental Models

A method of the invention may be used to create an animal or cell that may be used as a developmental model. Such a model may be used to study embryogenesis, organ development, organ system development, or the like. For instance, in one embodiment, a method of the invention may be used to create an animal or cell that comprises a chromosomal edit in one or more nucleic acid sequences associated with the development of an organ or organ system. Non-limiting examples of organs include the brain, eyes, nose, ears, throat, mouth (including teeth, tongue, lips, gums), spinal cord, bones, heart, blood vessels, lungs, liver, pancreas, gall bladder, spleen, esophagus, stomach, small intestines, large intestines, appendix, rectum, bladder, organs of the reproductive system, organs of the immune system (including thyroid, lymph nodes, lymph vessels), and organs of the endocrine system. Non-limiting examples of organ systems include the nervous system, the circulatory system, the digestive system, the respiratory system, the skeletal system, the lymphatic system, the reproductive system, the muscular system, the integumentary system, the excretory system, and the endocrine system.

In one embodiment, a method of the invention may be used to create an animal or cell in which at least one chromosomal sequence associated with neurodevelopment has been edited. Suitable chromosomal edits may include, but are not limited to, the type of edits detailed in section I(f) above. A chromosomal sequence associated with neurodevelopment may be a protein coding sequence or a control sequence. In certain embodiments, a neurodevelopmental sequence may be associated with a neurodevelopmental disorder, with biochemical pathways associated with a neurodevelopmental disorder, or associated with a disorder such as phenylketonuria that is closely associated with neurodevelopmental disorders.

Non-limiting examples of neurodevelopmental-associated sequences include A2BP1 [ataxin 2-binding protein 1], AADAT [aminoadipate aminotransferase], AANAT [arylalkylamine N-acetyltransferase], ABAT [4-aminobutyrate aminotransferase], ABCA1 [ATP-binding cassette, sub-family A (ABC1), member 1], ABCA13 [ATP-binding cassette, sub-family A (ABC1), member 13], ABCA2 [ATP-binding cassette, sub-family A (ABC1), member 2], ABCB1 [ATP-binding cassette, sub-family B (MDR/TAP), member 1], ABCB11 [ATP-binding cassette, sub-family B (MDR/TAP), member 11], ABCB4 [ATP-binding cassette, sub-family B (MDR/TAP), member 4], ABCB6 [ATP-binding cassette, sub-family B (MDR/TAP), member 6], ABCB7 [ATP-binding cassette, sub-family B (MDR/TAP), member 7], ABCC1 [ATP-binding cassette, sub-family C(CFTR/MRP), member 1], ABCC2 [ATP-binding cassette, sub-family C (CFTR/MRP), member 2], ABCC3 [ATP-binding cassette, sub-family C (CFTR/MRP), member 3], ABCC4 [ATP-binding cassette, sub-family C (CFTR/MRP), member 4], ABCD1 [ATP-binding cassette, sub-family D (ALD), member 1], ABCD3 [ATP-binding cassette, sub-family D (ALD), member 3], ABCG1 [ATP-binding cassette, sub-family G (WHITE), member 1], ABCC2 [ATP-binding cassette, sub-family G (WHITE), member 2], ABCC4 [ATP-binding cassette, sub-family G (WHITE), member 4], ABHD11 [abhydrolase domain containing 11], ABI1 [abl-interactor 1], ABL1 [c-abl oncogene 1, receptor tyrosine kinase], ABL2 [v-abl Abelson murine leukemia viral oncogene homolog 2 (arg, Abelson-related gene)], ABLIM1 [actin binding LIM protein 1], ABLIM2 [actin binding LIM protein family, member 2], ABLIM3 [actin binding LIM protein family, member 3], ABO [ABO blood group (transferase A, alpha 1-3-N-acetylgalactosaminyltransferase; transferase B, alpha 1-3-galactosyltransferase)], ACAA1 [acetyl-Coenzyme A acyltransferase 1], ACACA [acetyl-Coenzyme A carboxylase alpha], ACACB [acetyl-Coenzyme A carboxylase beta], ACADL [acyl-Coenzyme A dehydrogenase, long chain], ACADM [acyl-Coenzyme A dehydrogenase, C-4 to C-12 straight chain], ACADS [acyl-Coenzyme A dehydrogenase, C-2 to C-3 short chain], ACADSB [acyl-Coenzyme A dehydrogenase, short/branched chain], ACAN [aggrecan], ACAT2 [acetyl-Coenzyme A acetyltransferase 2], ACCN1 [amiloride-sensitive cation channel 1, neuronal], ACE [angiotensin I converting enzyme (peptidyl-dipeptidase A) 1], ACE2 [angiotensin I converting enzyme (peptidyl-dipeptidase A) 2], ACHE [acetylcholinesterase (Yt blood group)], ACLY [ATP citrate lyase], ACO1 [aconitase 1, soluble], ACTA1 [actin, alpha 1, skeletal muscle], ACTB [actin, beta], ACTC1 [actin, alpha, cardiac muscle 1], ACTG1 [actin, gamma 1], ACTL6A [actin-like 6A], ACTL6B [actin-like 6B], ACTN1 [actinin, alpha 1], ACTR1A [ARP1 actin-related protein 1 homolog A, centractin alpha (yeast)], ACTR2 [ARP2 actin-related protein 2 homolog (yeast)], ACTR3 [ARP3 actin-related protein 3 homolog (yeast)], ACTR3B [ARP3 actin-related protein 3 homolog B (yeast)], ACVR1 [activin A receptor, type I], ACVR2A [activin A receptor, type IIA], ADA [adenosine deaminase], ADAM10 [ADAM metallopeptidase domain 10], ADAM11 [ADAM metallopeptidase domain 11], ADAM12 [ADAM metallopeptidase domain 12], ADAM15 [ADAM metallopeptidase domain 15], ADAM17 [ADAM metallopeptidase domain 17], ADAM18 [ADAM metallopeptidase domain 18], ADAM19 [ADAM metallopeptidase domain 19 (meltrin beta)], ADAM2 [ADAM metallopeptidase domain 2], ADAM20 [ADAM metallopeptidase domain 20], ADAM21 [ADAM metallopeptidase domain 21], ADAM22 [ADAM metallopeptidase domain 22], ADAM23 [ADAM metallopeptidase domain 23], ADAM28 [ADAM metallopeptidase domain 28], ADAM29 [ADAM metallopeptidase domain 29], ADAM30 [ADAM metallopeptidase domain 30], ADAM8 [ADAM metallopeptidase domain 8], ADAMS [ADAM metallopeptidase domain 9 (meltrin gamma)], ADAMTS1 [ADAM metallopeptidase with thrombospondin type 1 motif, 1], ADAMTS13 [ADAM metallopeptidase with thrombospondin type 1 motif, 13], ADAMTS4 [ADAM metallopeptidase with thrombospondin type 1 motif, 4], ADAMTS5 [ADAM metallopeptidase with thrombospondin type 1 motif, 5], ADAP2 [ArfGAP with dual PH domains 2], ADAR [adenosine deaminase, RNA-specific], ADARB1 [adenosine deaminase, RNA-specific, B1 (RED1 homolog rat)], ADCY1 [adenylate cyclase 1 (brain)], ADCY10 [adenylate cyclase 10 (soluble)], ADCYAP1 [adenylate cyclase activating polypeptide 1 (pituitary)], ADD1 [adducin 1 (alpha)], ADD2 [adducin 2 (beta)], ADH1A [alcohol dehydrogenase 1A (class I), alpha polypeptide], ADIPOQ [adiponectin, C1Q and collagen domain containing], ADK [adenosine kinase], ADM [adrenomedullin], ADNP [activity-dependent neuroprotector homeobox], ADORA1 [adenosine A1 receptor], ADORA2A [adenosine A2a receptor], ADORA2B [adenosine A2b receptor], ADORA3 [adenosine A3 receptor], ADRA1B [adrenergic, alpha-1B-, receptor], ADRA2A [adrenergic, alpha-2A-, receptor], ADRA2B [adrenergic, alpha-2B-, receptor], ADRA2C [adrenergic, alpha-2C-, receptor], ADRB1 [adrenergic, beta-1-, receptor], ADRB2 [adrenergic, beta-2-, receptor, surface], ADRB3 [adrenergic, beta-3-, receptor], ADRBK2 [adrenergic, beta, receptor kinase 2], ADSL [adenylosuccinate lyase], AFF2 [AF4/FMR2 family, member 2], AFM [afamin], AFP [alpha-fetoprotein], AGAP1 [ArfGAP with GTPase domain, ankyrin repeat and PH domain 1], AGER [advanced glycosylation end product-specific receptor], AGFG1 [ArfGAP with FG repeats 1], AGPS [alkylglycerone phosphate synthase], AGRN [agrin], AGRP [agouti related protein homolog (mouse)], AGT [angiotensinogen (serpin peptidase inhibitor, clade A, member 8)], AGTR1 [angiotensin II receptor, type 1], AGTR2 [angiotensin II receptor, type 2], AHOY [adenosylhomocysteinase], AH11 [Abelson helper integration site 1], AHR [aryl hydrocarbon receptor], AHSG [alpha-2-HS-glycoprotein], AICDA [activation-induced cytidine deaminase], AIFM1 [apoptosis-inducing factor, mitochondrion-associated, 1], AIRE [autoimmune regulator], AKAP12 [A kinase (PRKA) anchor protein 12], AKAP9 [A kinase (PRKA) anchor protein (yotiao) 9], AKR1A1 [aldo-keto reductase family 1, member A1 (aldehyde reductase)], AKR1B1 [aldo-keto reductase family 1, member B1 (aldose reductase)], AKR1C3 [aldo-keto reductase family 1, member C3 (3-alpha hydroxysteroid dehydrogenase, type II)], AKT1 [v-akt murine thymoma viral oncogene homolog 1], AKT2 [v-akt murine thymoma viral oncogene homolog 2], AKT3 [v-akt murine thymoma viral oncogene homolog 3 (protein kinase B, gamma)], ALAD [aminolevulinate, delta-, dehydratase], ALB [albumin], ALB [albumin], ALCAM [activated leukocyte cell adhesion molecule], ALDH1A1 [aldehyde dehydrogenase 1 family, member A1], ALDH3A1 [aldehyde dehydrogenase 3 family, memberA1], ALDH5A1 [aldehyde dehydrogenase 5 family, member A1], ALDH7A1 [aldehyde dehydrogenase 7 family, member A1], ALDH9A1 [aldehyde dehydrogenase 9 family, member A1], ALDOA [aldolase A, fructose-bisphosphate], ALDOB [aldolase B, fructose-bisphosphate], ALDOC [aldolase C, fructose-bisphosphate], ALK [anaplastic lymphoma receptor tyrosine kinase], ALOX12 [arachidonate 12-lipoxygenase], ALOX5 [arachidonate 5-lipoxygenase], ALOX5AP [arachidonate 5-lipoxygenase-activating protein], ALPI [alkaline phosphatase, intestinal], ALPL [alkaline phosphatase, liver/bone/kidney], ALPP [alkaline phosphatase, placental (Regan isozyme)], ALS2 [amyotrophic lateral sclerosis 2 (juvenile)], AMACR [alpha-methylacyl-CoA racemase], AMBP [alpha-1-microglobulin/bikunin precursor], AMPH [amphiphysin], ANG [angiogenin, ribonuclease, RNase A family, 5], ANGPT1 [angiopoietin 1], ANGPT2 [angiopoietin 2], ANGPTL3 [angiopoietin-like 3], ANK1 [ankyrin 1, erythrocytic], ANK3 [ankyrin 3, node of Ranvier (ankyrin G)], ANKRD1 [ankyrin repeat domain 1 (cardiac muscle)], ANP32E [acidic (leucine-rich) nuclear phosphoprotein 32 family, member E], ANPEP [alanyl (membrane) aminopeptidase], ANXA1 [annexin A1], ANXA2 [annexin A2], ANXA5 [annexin A5], AP1S1 [adaptor-related protein complex 1, sigma 1 subunit], AP1S2 [adaptor-related protein complex 1, sigma 2 subunit], AP2A1 [adaptor-related protein complex 2, alpha 1 subunit], AP2B1 [adaptor-related protein complex 2, beta 1 subunit], APAF1 [apoptotic peptidase activating factor 1], APBA1 [amyloid beta (A4) precursor protein-binding, family A, member 1], APBA2 [amyloid beta (A4) precursor protein-binding, family A, member 2], APBB1 [amyloid beta (A4) precursor protein-binding, family B, member 1 (Fe65)], APBB2 [amyloid beta (A4) precursor protein-binding, family B, member 2], APC [adenomatous polyposis coli], APCS [amyloid P component, serum], APEX1 [APEX nuclease (multifunctional DNA repair enzyme) 1], APH1B [anterior pharynx defective 1 homolog B (C. elegans)], APLP1 [amyloid beta (A4) precursor-like protein 1], APOA1 [apolipoprotein A-I], APOA5 [apolipoprotein A-V], APOB [apolipoprotein B (including Ag(x) antigen)], APOC2 [apolipoprotein C-II], APOD [apolipoprotein D], APOE [apolipoprotein E], APOM [apolipoprotein M], APP [amyloid beta (A4) precursor protein], APPL1 [adaptor protein, phosphotyrosine interaction, PH domain and leucine zipper containing 1], APRT [adenine phosphoribosyltransferase], APTX [aprataxin], AQP1 [aquaporin 1 (Colton blood group)], AQP2 [aquaporin 2 (collecting duct)], AQP3 [aquaporin 3 (Gill blood group)], AQP4 [aquaporin 4], AR [androgen receptor], ARC [activity-regulated cytoskeleton-associated protein], AREG [amphiregulin], ARFGEF2 [ADP-ribosylation factor guanine nucleotide-exchange factor 2 (brefeldin A-inhibited)], ARG1 [arginase, liver], ARHGAP1 [Rho GTPase activating protein 1], ARHGAP32 [Rho GTPase activating protein 32], ARHGAP4 [Rho GTPase activating protein 4], ARHGAP5 [Rho GTPase activating protein 5], ARHGDIA [Rho GDP dissociation inhibitor (GDI) alpha], ARHGEF1 [Rho guanine nucleotide exchange factor (GEF) 1], ARHGEF10 [Rho guanine nucleotide exchange factor (GEF) 10], ARHGEF11 [Rho guanine nucleotide exchange factor (GEF) 11], ARHGEF12 [Rho guanine nucleotide exchange factor (GEF) 12], ARHGEF15 [Rho guanine nucleotide exchange factor (GEF) 15], ARHGEF16 [Rho guanine nucleotide exchange factor (GEF) 16], ARHGEF2 [Rho/Rac guanine nucleotide exchange factor (GEF) 2], ARHGEF3 [Rho guanine nucleotide exchange factor (GEF) 3], ARHGEF4 [Rho guanine nucleotide exchange factor (GEF) 4], ARHGEF5 [Rho guanine nucleotide exchange factor (GEF) 5], ARHGEF6 [Rac/Cdc42 guanine nucleotide exchange factor (GEF) 6], ARHGEF7 [Rho guanine nucleotide exchange factor (GEF) 7], ARHGEF9 [Cdc42 guanine nucleotide exchange factor (GEF) 9], ARID1A [AT rich interactive domain 1A (SWI-like)], ARID1B [AT rich interactive domain 1B (SWI1-like)], ARL13B [ADP-ribosylation factor-like 13B], ARPC1A [actin related protein 2/3 complex, subunit 1A, 41 kDa], ARPC1B [actin related protein 2/3 complex, subunit 1B, 41 kDa], ARPC2 [actin related protein 2/3 complex, subunit 2, 34 kDa], ARPC3 [actin related protein 2/3 complex, subunit 3, 21 kDa], ARPC4 [actin related protein 2/3 complex, subunit 4, 20 kDa], ARPC5 [actin related protein 2/3 complex, subunit 5, 16 kDa], ARPC5L [actin related protein 2/3 complex, subunit 5-like], ARPP19 [cAMP-regulated phosphoprotein, 19 kDa], ARR3 [arrestin 3, retinal (X-arrestin)], ARRB2 [arrestin, beta 2], ARSA [arylsulfatase A], ARTN [artemin], ARX [aristaless related homeobox], ASCL1 [achaete-scute complex homolog 1 (Drosophila)], ASMT [acetylserotonin O-methyltransferase], ASPA [aspartoacylase (Canavan disease)], ASPG [asparaginase homolog (S. cerevisiae)], ASPH [aspartate beta-hydroxylase], ASPM [asp (abnormal spindle) homolog, microcephaly associated (Drosophila)], ASRGL1 [asparaginase like 1], ASS1 [argininosuccinate synthase 1], ASTN1 [astrotactin 1], ATAD5 [ATPase family, AAA domain containing 5], ATF2 [activating transcription factor 2], ATF4 [activating transcription factor 4 (tax-responsive enhancer element B67)], ATF6 [activating transcription factor 6], ATM [ataxia telangiectasia mutated], ATOH1 [atonal homolog 1 (Drosophila)], ATOX1 [ATX1 antioxidant protein 1 homolog (yeast)], ATP10A [ATPase, class V, type 10A], ATP2A2 [ATPase, Ca++ transporting, cardiac muscle, slow twitch 2], ATP2B2 [ATPase, Ca++ transporting, plasma membrane 2], ATP2B4 [ATPase, Ca++ transporting, plasma membrane 4], ATP5O [ATP synthase, H+ transporting, mitochondrial F1 complex, O subunit], ATP6AP1 [ATPase, H+ transporting, lysosomal accessory protein 1], ATP6V0C [ATPase, H+ transporting, lysosomal 16 kDa, V0 subunit c], ATP7A [ATPase, Cu++ transporting, alpha polypeptide], ATP8A1 [ATPase, aminophospholipid transporter (APLT), class I, type 8A, member 1], ATR [ataxia telangiectasia and Rad3 related], ATRN [attractin], ATRX [alpha thalassemia/mental retardation syndrome X-linked (RAD54 homolog, S. cerevisiae)], ATXN1 [ataxin 1], ATXN2 [ataxin 2], ATXN3 [ataxin 3], AURKA [aurora kinase A], AUTS2 [autism susceptibility candidate 2], AVP [arginine vasopressin], AVPR1A [arginine vasopressin receptor 1A], AXIN2 [axin 2], AXL [AXL receptor tyrosine kinase], AZU1 [azurocidin 1], B2M [beta-2-microglobulin], B3GNT2 [UDP-GlcNAc:betaGal beta-1 [3-N-acetylglucosaminyltransferase 2], B9D1 [B9 protein domain 1], BACE1 [beta-site APP-cleaving enzyme 1], BACE2 [beta-site APP-cleaving enzyme 2], BACH1 [BTB and CNC homology 1, basic leucine zipper transcription factor 1], BAD [BCL2-associated agonist of cell death], BACE2 [B melanoma antigen family, member 2], BAIAP2 [BAI1-associated protein 2], BAIAP2L1 [BAI1-associated protein 2-like 1], BAK1 [BCL2-antagonist/killer 1], BARD1 [BRCA1 associated RING domain 1], BARHL1 [BarH-like homeobox 1], BARHL2 [BarH-like homeobox 2], BASP1 [brain abundant, membrane attached signal protein 1], BAX [BCL2-associated X protein], BAZ1A [bromodomain adjacent to zinc finger domain, 1A], BAZ1B [bromodomain adjacent to zinc finger domain, 1B], BBS9 [Bardet-Biedl syndrome 9], BCAR1 [breast cancer anti-estrogen resistance 1], BCHE [butyrylcholinesterase], BCL10 [B-cell CLL/lymphoma 10], BCL2 [B-cell CLL/lymphoma 2], BCL2A1 [BCL2-related protein A1], BCL2L1 [BCL2-like 1], BCL2L11 [BCL2-like 11 (apoptosis facilitator)], BCL3 [B-cell CLL/lymphoma 3], BCL6 [B-cell CLL/lymphoma 6], BCL7A [B-cell CLL/lymphoma 7A], BCL7B [B-cell CLL/lymphoma 7B], BCL7C [B-cell CLL/lymphoma 70], BCR [breakpoint cluster region], BDKRB1 [bradykinin receptor B1], BDNF [brain-derived neurotrophic factor], BECN1 [beclin 1, autophagy related], BEST1 [bestrophin 1], BEX1 [brain expressed, X-linked 1], BEX2 [brain expressed X-linked 2], BGLAP [bone gamma-carboxyglutamate (gla) protein], BGN [biglycan], BID [BH3 interacting domain death agonist], BIN1 [bridging integrator 1], BIRC2 [baculoviral IAP repeat-containing 2], BIRC3 [baculoviral IAP repeat-containing 3], BIRC5 [baculoviral IAP repeat-containing 5], BIRC7 [baculoviral IAP repeat-containing 7], BLK [B lymphoid tyrosine kinase], BLVRB [biliverdin reductase B (flavin reductase (NADPH))], BMI1 [BMI1 polycomb ring finger oncogene], BMP1 [bone morphogenetic protein 1], BMP10 [bone morphogenetic protein 10], BMP15 [bone morphogenetic protein 15], BMP2 [bone morphogenetic protein 2], BMP3 [bone morphogenetic protein 3], BMP4 [bone morphogenetic protein 4], BMP5 [bone morphogenetic protein 5], BMP6 [bone morphogenetic protein 6], BMP7 [bone morphogenetic protein 7], BMP8A [bone morphogenetic protein 8a], BMP8B [bone morphogenetic protein 8b], BMPR1A [bone morphogenetic protein receptor, type IA], BMPR1B [bone morphogenetic protein receptor, type IB], BMPR2 [bone morphogenetic protein receptor, type II (serine/threonine kinase)], BOC [Boc homolog (mouse)], BOK [BCL2-related ovarian killer], BPI [bactericidal/permeability-increasing protein], BRAF [v-raf murine sarcoma viral oncogene homolog B1], BRCA1 [breast cancer 1, early onset], BRCA2 [breast cancer 2, early onset], BRWD1 [bromodomain and WD repeat domain containing 1], BSND [Bartter syndrome, infantile, with sensorineural deafness (Barttin)], BST2 [bone marrow stromal cell antigen 2], BTBD10 [BTB (POZ) domain containing 10], BTC [betacellulin], BTD [biotinidase], BTG3 [BTG family, member 3], BTK [Bruton agammaglobulinemia tyrosine kinase], BTN1A1 [butyrophilin, subfamily 1, member A1], BUB1B [budding uninhibited by benzimidazoles 1 homolog beta (yeast)], C15orf2 [chromosome 15 open reading frame 2], C16 or 175 [chromosome 16 open reading frame 75], C17orf42 [chromosome 17 open reading frame 42], C1orf187 [chromosome 1 open reading frame 187], C1R [complement component 1, r subcomponent], C1S [complement component 1, s subcomponent], C21orf2 [chromosome 21 open reading frame 2], C21orf33 [chromosome 21 open reading frame 33], C21orf45 [chromosome 21 open reading frame 45], C21orf62 [chromosome 21 open reading frame 62], C21orf74 [chromosome 21 open reading frame 74], C3 [complement component 3], C3orf58 [chromosome 3 open reading frame 58], C4A [complement component 4A (Rodgers blood group)], C4B [complement component 4B (Chido blood group)], C5AR1 [complement component 5a receptor 1], C6orf106 [chromosome 6 open reading frame 106], C6orf25 [chromosome 6 open reading frame 25], CA1 [carbonic anhydrase I], CA2 [carbonic anhydrase II], CA3 [carbonic anhydrase III, muscle specific], CA6 [carbonic anhydrase VI], CA9 [carbonic anhydrase IX], CABIN1 [calcineurin binding protein 1], CABLES1 [Cdk5 and Abl enzyme substrate 1], CACNA1B [calcium channel, voltage-dependent, N type, alpha 1B subunit], CACNA1C [calcium channel, voltage-dependent, L type, alpha 1C subunit], CACNA1G [calcium channel, voltage-dependent, T type, alpha 1G subunit], CACNA1H [calcium channel, voltage-dependent, T type, alpha 1H subunit], CACNA2D1 [calcium channel, voltage-dependent, alpha 2/delta subunit 1], CADM1 [cell adhesion molecule 1], CADPS2 [Ca-++-dependent secretion activator 2], CALB2 [calbindin 2], CALCA [calcitonin-related polypeptide alpha], CALCR [calcitonin receptor], CALM3 [calmodulin 3 (phosphorylase kinase, delta)], CALR [calreticulin], CAMK1 [calcium/calmodulin-dependent protein kinase I], CAMK2A [calcium/calmodulin-dependent protein kinase II alpha], CAMK2B [calcium/calmodulin-dependent protein kinase II beta], CAMK2G [calcium/calmodulin-dependent protein kinase II gamma], CAMK4 [calcium/calmodulin-dependent protein kinase IV], CAMKK2 [calcium/calmodulin-dependent protein kinase kinase 2, beta], CAMP [cathelicidin antimicrobial peptide], CANT1 [calcium activated nucleotidase 1], CANX [calnexin], CAPN1 [calpain 1, (mu/I) large subunit], CAPN2 [calpain 2, (m/II) large subunit], CAPN5 [calpain 5], CAPZA1 [capping protein (actin filament) muscle Z-line, alpha 1], CARD16 [caspase recruitment domain family, member 16], CARM1 [coactivator-associated arginine methyltransferase 1], CARTPT [CART prepropeptide], CASK [calcium/calmodulin-dependent serine protein kinase (MAGUK family)], CASP1 [caspase 1, apoptosis-related cysteine peptidase (interleukin 1, beta, convertase)], CASP10 [caspase 10, apoptosis-related cysteine peptidase], CASP2 [caspase 2, apoptosis-related cysteine peptidase], CASP3 [caspase 3, apoptosis-related cysteine peptidase], CASP6 [caspase 6, apoptosis-related cysteine peptidase], CASP7 [caspase 7, apoptosis-related cysteine peptidase], CASP8 [caspase 8, apoptosis-related cysteine peptidase], CASP8AP2 [caspase 8 associated protein 2], CASP9 [caspase 9, apoptosis-related cysteine peptidase], CASR [calcium-sensing receptor], CAST [calpastatin], CAT [catalase], CAV1 [caveolin 1, caveolae protein, 22 kDa], CAV2 [caveolin 2], CAV3 [caveolin 3], CBL [Cas-Br-M (murine) ecotropic retroviral transforming sequence], CBLB [Cas-Br-M (murine) ecotropic retroviral transforming sequence b], CBR1 [carbonyl reductase 1], CBR3 [carbonyl reductase 3], CBS [cystathionine-beta-synthase], CBX1 [chromobox homolog 1 (HP1 beta homolog Drosophila)], CBX5 [chromobox homolog 5 (HP1 alpha homolog, Drosophila)], CC2D2A [coiled-coil and C2 domain containing 2A], CCBE1 [collagen and calcium binding EGF domains 1], CCBL1 [cysteine conjugate-beta lyase, cytoplasmic], CCDC50 [coiled-coil domain containing 50], CCK [cholecystokinin], CCKAR [cholecystokinin A receptor], CCL1 [chemokine (C-C motif) ligand 1], CCL11 [chemokine (C-C motif) ligand 11], CCL13 [chemokine (C-C motif) ligand 13], CCL17 [chemokine (C-C motif) ligand 17], CCL19 [chemokine (C-C motif) ligand 19], CCL2 [chemokine (C-C motif) ligand 2], CCL20 [chemokine (C-C motif) ligand 20], CCL21 [chemokine (C-C motif) ligand 21], CCL22 [chemokine (C-C motif) ligand 22], CCL26 [chemokine (C-C motif) ligand 26], CCL27 [chemokine (C-C motif) ligand 27], CCL3 [chemokine (C-C motif) ligand 3], CCL4 [chemokine (C-C motif) ligand 4], CCL5 [chemokine (C-C motif) ligand 5], CCL7 [chemokine (C-C motif) ligand 7], CCL8 [chemokine (C-C motif) ligand 8], CCNA1 [cyclin A1], CCNA2 [cyclin A2], CCNB1 [cyclin B1], CCND1 [cyclin D1], CCND2 [cyclin D2], CCND3 [cyclin D3], CCNG1 [cyclin G1], CCNH [cyclin H], CCNT1 [cyclin T1], CCR1 [chemokine (C-C motif) receptor 1], CCR3 [chemokine (C-C motif) receptor 3], CCR4 [chemokine (C-C motif) receptor 4], CCR5 [chemokine (C-C motif) receptor 5], CCR6 [chemokine (C-C motif) receptor 6], CCR7 [chemokine (C-C motif) receptor 7], CCT5 [chaperonin containing TCP1, subunit 5 (epsilon)], CD14 [CD14 molecule], CD19 [CD19 molecule], CD1A [CD1a molecule], CD1B [CD1b molecule], CD1D [CD1d molecule], CD2 [CD2 molecule], CD209 [CD209 molecule], CD22 [CD22 molecule], CD244 [CD244 molecule, natural killer cell receptor 2B4], CD247 [CD247 molecule], CD27 [CD27 molecule], CD274 [CD274 molecule], CD28 [CD28 molecule], CD2AP [CD2-associated protein], CD33 [CD33 molecule], CD34 [CD34 molecule], CD36 [CD36 molecule (thrombospondin receptor)], CD3E [CD3e molecule, epsilon (CD3-TCR complex)], CD3G [CD3g molecule, gamma (CD3-TCR complex)], CD4 [CD4 molecule], CD40 [CD40 molecule, TNF receptor superfamily member 5], CD40LG [CD40 ligand], CD44 [CD44 molecule (Indian blood group)], CD46 [CD46 molecule, complement regulatory protein], CD47 [CD47 molecule], CD5 [CD5 molecule], CD55 [CD55 molecule, decay accelerating factor for complement (Cromer blood group)], CD58 [CD58 molecule], CD59 [CD59 molecule, complement regulatory protein], CD63 [CD63 molecule], CD69 [CD69 molecule], CD7 [CD7 molecule], CD72 [CD72 molecule], CD74 [CD74 molecule, major histocompatibility complex, class II invariant chain], CD79A [CD79a molecule, immunoglobulin-associated alpha], CD79B [CD79b molecule, immunoglobulin-associated beta], CD80 [CD80 molecule], CD81 [CD81 molecule], CD86 [CD86 molecule], CD8A [CD8a molecule], CD9 [CD9 molecule], CD99 [CD99 molecule], CDA [cytidine deaminase], CDC25A [cell division cycle 25 homolog A (S. pombe)], CDC25C [cell division cycle 25 homolog C (S. pombe)], CDC37 [cell division cycle 37 homolog (S. cerevisiae)], CDC42 [cell division cycle 42 (GTP binding protein, 25 kDa)], CDC5L [CDC5 cell division cycle 5-like (S. pombe)], CDH1 [cadherin 1, type 1, E-cadherin (epithelial)], CDH10 [cadherin 10, type 2 (T2-cadherin)], CDH12 [cadherin 12, type 2 (N-cadherin 2)], CDH15 [cadherin 15, type 1, M-cadherin (myotubule)], CDH2 [cadherin 2, type 1, N-cadherin (neuronal)], CDH4 [cadherin 4, type 1, R-cadherin (retinal)], CDH5 [cadherin 5, type 2 (vascular endothelium)], CDH9 [cadherin 9, type 2 (T1-cadherin)], CDIPT [CDP-diacylglycerol-inositol 3-phosphatidyltransferase (phosphatidylinositol synthase)], CDK1 [cyclin-dependent kinase 1], CDK14 [cyclin-dependent kinase 14], CDK2 [cyclin-dependent kinase 2], CDK4 [cyclin-dependent kinase 4], CDK5 [cyclin-dependent kinase 5], CDK5R1 [cyclin-dependent kinase 5, regulatory subunit 1 (p35)], CDK5RAP2 [CDK5 regulatory subunit associated protein 2], CDK6 [cyclin-dependent kinase 6], CDK7 [cyclin-dependent kinase 7], CDK9 [cyclin-dependent kinase 9], CDKL5 [cyclin-dependent kinase-like 5], CDKN1A [cyclin-dependent kinase inhibitor 1A (p21, Cip1)], CDKN1B [cyclin-dependent kinase inhibitor 1B (p27, Kip1)], CDKN1C [cyclin-dependent kinase inhibitor 1C (p57, Kip2)], CDKN2A [cyclin-dependent kinase inhibitor 2A (melanoma, p16, inhibits CDK4)], CDKN2B [cyclin-dependent kinase inhibitor 2B (p15, inhibits CDK4)], CDKN2C [cyclin-dependent kinase inhibitor 2C (p18, inhibits CDK4)], CDKN2D [cyclin-dependent kinase inhibitor 2D (p19, inhibits CDK4)], CDNF [cerebral dopamine neurotrophic factor], CDO1 [cysteine dioxygenase, type I], CDR2 [cerebellar degeneration-related protein 2, 62 kDa], CDT1 [chromatin licensing and DNA replication factor 1], CDX1 [caudal type homeobox 1], CDX2 [caudal type homeobox 2], CEACAM1 [carcinoembryonic antigen-related cell adhesion molecule 1 (biliary glycoprotein)], CEACAM3 [carcinoembryonic antigen-related cell adhesion molecule 3], CEACAM5 [carcinoembryonic antigen-related cell adhesion molecule 5], CEACAM7 [carcinoembryonic antigen-related cell adhesion molecule 7], CEBPB [CCAAT/enhancer binding protein (C/EBP), beta], CEBPD [CCAAT/enhancer binding protein (C/EBP), delta], CECR2 [cat eye syndrome chromosome region, candidate 2], CEL [carboxyl ester lipase (bile salt-stimulated lipase)], CENPC1 [centromere protein C1], CENPJ [centromere protein J], CEP290 [centrosomal protein 290 kDa], CER1 [cerberus 1, cysteine knot superfamily, homolog (Xenopus laevis)], CETP [cholesteryl ester transfer protein, plasma], CFC1 [cripto, FRL-1, cryptic family 1], CFH [complement factor H], CFHR1 [complement factor H-related 1], CFHR3 [complement factor H-related 3], CFHR4 [complement factor H-related 4], CFI [complement factor I], CFL1 [cofilin 1 (non-muscle)], CFL2 [cofilin 2 (muscle)], CFLAR [CASP8 and FADD-like apoptosis regulator], CFTR [cystic fibrosis transmembrane conductance regulator (ATP-binding cassette sub-family C, member 7)], CGA [glycoprotein hormones, alpha polypeptide], CGB [chorionic gonadotropin, beta polypeptide], CGB5 [chorionic gonadotropin, beta polypeptide 5], CGGBP1 [CGG triplet repeat binding protein 1], CHAF1A [chromatin assembly factor 1, subunit A (p150)], CHAF1B [chromatin assembly factor 1, subunit B (p60)], CHAT [choline acetyltransferase], CHEK1 [CHK1 checkpoint homolog (S. pombe)], CHEK2 [CHK2 checkpoint homolog (S. pombe)], CHGA [chromogranin A (parathyroid secretory protein 1)], CHKA [choline kinase alpha], CHL1 [cell adhesion molecule with homology to L1CAM (close homolog of L1)], CHN1 [chimerin (chimaerin) 1], CHP [calcium binding protein P22], CHP2 [calcineurin B homologous protein 2], CHRD [chordin], CHRM1 [cholinergic receptor, muscarinic 1], CHRM2 [cholinergic receptor, muscarinic 2], CHRM3 [cholinergic receptor, muscarinic 3], CHRM5 [cholinergic receptor, muscarinic 5], CHRNA3 [cholinergic receptor, nicotinic, alpha 3], CHRNA4 [cholinergic receptor, nicotinic, alpha 4], CHRNA7 [cholinergic receptor, nicotinic, alpha 7], CHRNB2 [cholinergic receptor, nicotinic, beta 2 (neuronal)], CHST1 [carbohydrate (keratan sulfate Gal-6) sulfotransferase 1], CHST10 [carbohydrate sulfotransferase 10], CHST3 [carbohydrate (chondroitin 6) sulfotransferase 3], CHUK [conserved helix-loop-helix ubiquitous kinase], CHURC1 [churchill domain containing 1], CIB1 [calcium and integrin binding 1 (calmyrin)], CIITA [class II, major histocompatibility complex, transactivator], CIRBP [cold inducible RNA binding protein], CISD1 [CDGSH iron sulfur domain 1], CISH [cytokine inducible SH2-containing protein], CIT [citron (rho-interacting, serine/threonine kinase 21)], CLASP2 [cytoplasmic linker associated protein 2], CLCF1 [cardiotrophin-like cytokine factor 1], CLCN2 [chloride channel 2], CLDN1 [claudin 1], CLDN14 [claudin 14], CLDN16 [claudin 16], CLDN3 [claudin 3], CLDN4 [claudin 4], CLDN5 [claudin 5], CLDN8 [claudin 8], CLEC12A [C-type lectin domain family 12, member A], CLEC16A [C-type lectin domain family 16, member A], CLEC5A [C-type lectin domain family 5, member A], CLEC7A [C-type lectin domain family 7, member A], CLIP2 [CAP-GLY domain containing linker protein 2], CLSTN1 [calsyntenin 1], CLTC [clathrin, heavy chain (Hc)], CLU [clusterin], CMIP [c-Maf-inducing protein], CNBP [CCHC-type zinc finger, nucleic acid binding protein], CNGA3 [cyclic nucleotide gated channel alpha 3], CNGB3 [cyclic nucleotide gated channel beta 3], CNN1 [calponin 1, basic, smooth muscle], CNN2 [calponin 2], CNN3 [calponin 3, acidic], CNOT8 [CCR4-NOT transcription complex, subunit 8], CNP [2′ [3′-cyclic nucleotide 3′ phosphodiesterase], CNR1 [cannabinoid receptor 1 (brain)], CNR2 [cannabinoid receptor 2 (macrophage)], CNTF [ciliary neurotrophic factor], CNTFR [ciliary neurotrophic factor receptor], CNTFR [ciliary neurotrophic factor receptor], CNTFR [ciliary neurotrophic factor receptor], CNTLN [centlein, centrosomal protein], CNTN1 [contactin 1], CNTN2 [contactin 2 (axonal)], CNTN4 [contactin 4], CNTNAP1 [contactin associated protein 1], CNTNAP2 [contactin associated protein-like 2], COBL [cordon-bleu homolog (mouse)], COG2 [component of oligomeric golgi complex 2], COL18A1 [collagen, type XVIII, alpha 1], COL1A1 [collagen, type I, alpha 1], COL1A2 [collagen, type I, alpha 2], COL2A1 [collagen, type II, alpha 1], COL3A1 [collagen, type III, alpha 1], COL4A3 [collagen, type IV, alpha 3 (Goodpasture antigen)], COL4A3BP [collagen, type IV, alpha 3 (Goodpasture antigen) binding protein], COL5A1 [collagen, type V, alpha 1], COL5A2 [collagen, type V, alpha 2], COL6A1 [collagen, type VI, alpha 1], COL6A2 [collagen, type VI, alpha 2], COL6A3 [collagen, type VI, alpha 3], COMT [catechol-O-methyltransferase], COPG2 [coatomer protein complex, subunit gamma 2], COPS4 [COPS constitutive photomorphogenic homolog subunit 4 (Arabidopsis)], CORO1A [coronin, actin binding protein, 1A], COX5A [cytochrome c oxidase subunit Va], COX7B [cytochrome c oxidase subunit VIIb], CP [ceruloplasmin (ferroxidase)], CPA1 [carboxypeptidase A1 (pancreatic)], CPA2 [carboxypeptidase A2 (pancreatic)], CPA5 [carboxypeptidase A5], CPB2 [carboxypeptidase B2 (plasma)], CPOX [coproporphyrinogen oxidase], CPS1 [carbamoyl-phosphate synthetase 1, mitochondrial], CPT1A [carnitine palmitoyltransferase 1A (liver)], CR1 [complement component (3b/4b) receptor 1 (Knops blood group)], CR2 [complement component (3d/Epstein Barr virus) receptor 2], CRABP1 [cellular retinoic acid binding protein 1], CRABP2 [cellular retinoic acid binding protein 2], CRAT [carnitine O-acetyltransferase], CRB1 [crumbs homolog 1 (Drosophila)], CREB1 [cAMP responsive element binding protein 1], CREBBP [CREB binding protein], CRELD1 [cysteine-rich with EGF-like domains 1], CRH [corticotropin releasing hormone], CRIP1 [cysteine-rich protein 1 (intestinal)], CRK [v-crk sarcoma virus CT10 oncogene homolog (avian)], CRKL [v-crk sarcoma virus CT10 oncogene homolog (avian)-like], CRLF1 [cytokine receptor-like factor 1], CRLF2 [cytokine receptor-like factor 2], CRLF3 [cytokine receptor-like factor 3], CRMP1 [collapsin response mediator protein 1], CRP [C-reactive protein, pentraxin-related], CRTC1 [CREB regulated transcription coactivator 1], CRX [cone-rod homeobox], CRYAA [crystallin, alpha A], CRYAB [crystallin, alpha B], CS [citrate synthase], CSAD [cysteine sulfinic acid decarboxylase], CSF1 [colony stimulating factor 1 (macrophage)], CSF1R [colony stimulating factor 1 receptor], CSF2 [colony stimulating factor 2 (granulocyte-macrophage)], CSF2RA [colony stimulating factor 2 receptor, alpha, low-affinity (granulocyte-macrophage)], CSF3 [colony stimulating factor 3 (granulocyte)], CSF3R [colony stimulating factor 3 receptor (granulocyte)], CSH2 [chorionic somatomammotropin hormone 2], CSK [c-src tyrosine kinase], CSMD1 [CUB and Sushi multiple domains 1], CSMD3 [CUB and Sushi multiple domains 3], CSNK1D [casein kinase 1, delta], CSNK1E [casein kinase 1, epsilon], CSNK2A1 [casein kinase 2, alpha 1 polypeptide], CSPG4 [chondroitin sulfate proteoglycan 4], CSPG5 [chondroitin sulfate proteoglycan 5 (neuroglycan C)], CST3 [cystatin C], CST7 [cystatin F (leukocystatin)], CSTB [cystatin B (stefin B)], CTAG1B [cancer/testis antigen 1B], CTBP1 [C-terminal binding protein 1], CTCF [CCCTC-binding factor (zinc finger protein)], CTDSP1 [CTD (carboxy-terminal domain, RNA polymerase II, polypeptide A) small phosphatase 1], CTF1 [cardiotrophin 1], CTGF [connective tissue growth factor], CTLA4 [cytotoxic T-lymphocyte-associated protein 4], CTNNA1 [catenin (cadherin-associated protein), alpha 1, 102 kDa], CTNNAL1 [catenin (cadherin-associated protein), alpha-like 1], CTNNB1 [catenin (cadherin-associated protein), beta 1, 88 kDa], CTNND1 [catenin (cadherin-associated protein), delta 1], CTNND2 [catenin (cadherin-associated protein), delta 2 (neural plakophilin-related arm-repeat protein)], CTNS [cystinosis, nephropathic], CTRL [chymotrypsin-like], CTSB [cathepsin B], CTSC [cathepsin C], CTSD [cathepsin D], CTSG [cathepsin G], CTSH [cathepsin H], CTSL1 [cathepsin L1], CTSS [cathepsin S], CTTN [cortactin], CTTNBP2 [cortactin binding protein 2], CUL4B [cullin 4B], CUL5 [cullin 5], CUX2 [cut-like homeobox 2], CX3CL1 [chemokine (C-X3-C motif) ligand 1], CX3CR1 [chemokine (C-X3-C motif) receptor 1], CXADR [coxsackie virus and adenovirus receptor], CXCL1 [chemokine (C-X-C motif) ligand 1 (melanoma growth stimulating activity, alpha)], CXCL10 [chemokine (C-X-C motif) ligand 10], CXCL12 [chemokine (C-X-C motif) ligand 12 (stromal cell-derived factor 1)], CXCL16 [chemokine (C-X-C motif) ligand 16], CXCL2 [chemokine (C-X-C motif) ligand 2], CXCL5 [chemokine (C-X-C motif) ligand 5], CXCR1 [chemokine (C-X-C motif) receptor 1], CXCR2 [chemokine (C-X-C motif) receptor 2], CXCR3 [chemokine (C-X-C motif) receptor 3], CXCR4 [chemokine (C-X-C motif) receptor 4], CXCR5 [chemokine (C-X-C motif) receptor 5], CYB5A [cytochrome b5 type A (microsomal)], CYBA [cytochrome b-245, alpha polypeptide], CYBB [cytochrome b-245, beta polypeptide], CYCS [cytochrome c, somatic], CYFIP1 [cytoplasmic FMR1 interacting protein 1], CYLD [cylindromatosis (turban tumor syndrome)], CYP11A1 [cytochrome P450, family 11, subfamily A, polypeptide 1], CYP11B1 [cytochrome P450, family 11, subfamily B, polypeptide 1], CYP11B2 [cytochrome P450, family 11, subfamily B, polypeptide 2], CYP17A1 [cytochrome P450, family 17, subfamily A, polypeptide 1], CYP19A1 [cytochrome P450, family 19, subfamily A, polypeptide 1], CYP1A1 [cytochrome P450, family 1, subfamily A, polypeptide 1], CYP1A2 [cytochrome P450, family 1, subfamily A, polypeptide 2], CYP1B1 [cytochrome P450, family 1, subfamily B, polypeptide 1], CYP21A2 [cytochrome P450, family 21, subfamily A, polypeptide 2], CYP2A6 [cytochrome P450, family 2, subfamily A, polypeptide 6], CYP2B6 [cytochrome P450, family 2, subfamily B, polypeptide 6], CYP2C9 [cytochrome P450, family 2, subfamily C, polypeptide 9], CYP2D6 [cytochrome P450, family 2, subfamily D, polypeptide 6], CYP2E1 [cytochrome P450, family 2, subfamily E, polypeptide 1], CYP3A4 [cytochrome P450, family 3, subfamily A, polypeptide 4], CYP7A1 [cytochrome P450, family 7, subfamily A, polypeptide 1], CYR61 [cysteine-rich, angiogenic inducer, 61], CYSLTR1 [cysteinyl leukotriene receptor 1], CYSLTR2 [cysteinyl leukotriene receptor 2], DAB1 [disabled homolog 1 (Drosophila)], DAGLA [diacylglycerol lipase, alpha], DAGLB [diacylglycerol lipase, beta], DAO [D-amino-acid oxidase], DAOA [D-amino acid oxidase activator], DAPK1 [death-associated protein kinase 1], DAPK3 [death-associated protein kinase 3], DAXX [death-domain associated protein], DBH [dopamine beta-hydroxylase (dopamine beta-monooxygenase)], DBI [diazepam binding inhibitor (GABA receptor modulator, acyl-Coenzyme A binding protein)], DBN1 [drebrin 1], DCAF6 [DDB1 and CUL4 associated factor 6], DCC [deleted in colorectal carcinoma], DCDC2 [doublecortin domain containing 2], DCK [deoxycytidine kinase], DCLK1 [doublecortin-like kinase 1], DCN [decorin], DCTN1 [dynactin 1 (p150, glued homolog, Drosophila)], DCTN2 [dynactin 2 (p50)], DCTN4 [dynactin 4 (p62)], DCUN1D1 [DCN1, defective in cullin neddylation 1, domain containing 1 (S. cerevisiae)], DCX [doublecortin], DDB1 [damage-specific DNA binding protein 1, 127 kDa], DDC [dopa decarboxylase (aromatic L-amino acid decarboxylase)], DDIT3 [DNA-damage-inducible transcript 3], DDIT4 [DNA-damage-inducible transcript 4], DDIT4L [DNA-damage-inducible transcript 4-like], DDR1 [discoidin domain receptor tyrosine kinase 1], DDX10 [DEAD (Asp-Glu-Ala-Asp) box polypeptide 10], DDX17 [DEAD (Asp-Glu-Ala-Asp) box polypeptide 17], DEFB4A [defensin, beta 4A], DEK [DEK oncogene], DES [desmin], DEXI [Dexi homolog (mouse)], DFFA [DNA fragmentation factor, 45 kDa, alpha polypeptide], DFNB31 [deafness, autosomal recessive 31], DGCR6 [DiGeorge syndrome critical region gene 6], DGUOK [deoxyguanosine kinase], DHCR7 [7-dehydrocholesterol reductase], DHFR [dihydrofolate reductase], DIAPH1 [diaphanous homolog 1 (Drosophila)], DICER1 [dicer 1, ribonuclease type III], D101 [deiodinase, iodothyronine, type I], D102 [deiodinase, iodothyronine, type II], DIP2A [DIP2 disco-interacting protein 2 homolog A (Drosophila)], DIRAS3 [DIRAS family, GTP-binding RAS-like 3], DISC1 [disrupted in schizophrenia 1], DISC2 [disrupted in schizophrenia 2 (non-protein coding)], DKC1 [dyskeratosis congenita 1, dyskerin], DLG1 [discs, large homolog 1 (Drosophila)], DLG2 [discs, large homolog 2 (Drosophila)], DLG3 [discs, large homolog 3 (Drosophila)], DLG4 [discs, large homolog 4 (Drosophila)], DLGAP1 [discs, large (Drosophila) homolog-associated protein 1], DLGAP2 [discs, large (Drosophila) homolog-associated protein 2], DLK1 [delta-like 1 homolog (Drosophila)], DLL1 [delta-like 1 (Drosophila)], DLX1 [distal-less homeobox 1], DLX2 [distal-less homeobox 2], DLX3 [distal-less homeobox 3], DLX4 [distal-less homeobox 4], DLX5 [distal-less homeobox 5], DLX6 [distal-less homeobox 6], DMBT1 [deleted in malignant brain tumors 1], DMC1 [DMC1 dosage suppressor of mck1 homolog, meiosis-specific homologous recombination (yeast)], DMD [dystrophin], DMPK [dystrophia myotonica-protein kinase], DNAI2 [dynein, axonemal, intermediate chain 2], DNAJC28 [DnaJ (Hsp40) homolog, subfamily C, member 28], DNAJC30 [DnaJ (Hsp40) homolog, subfamily C, member 30], DNASE1 [deoxyribonuclease I], DNER [delta/notch-like EGF repeat containing], DNLZ [DNL-type zinc finger], DNM1 [dynamin 1], DNM3 [dynamin 3], DNMT1 [DNA (cytosine-5-)-methyltransferase 1], DNMT3A [DNA (cytosine-5-)-methyltransferase 3 alpha], DNMT3B [DNA (cytosine-5-)-methyltransferase 3 beta], DNTT [deoxynucleotidyltransferase, terminal], DOC2A [double C2-like domains, alpha], DOCK1 [dedicator of cytokinesis 1], DOCK3 [dedicator of cytokinesis 3], DOCK4 [dedicator of cytokinesis 4], DOCK7 [dedicator of cytokinesis 7], DOK7 [docking protein 7], DONSON [downstream neighbor of SON], DOPEY1 [dopey family member 1], DOPEY2 [dopey family member 2], DPF1 [D4, zinc and double PHD fingers family 1], DPF3 [D4, zinc and double PHD fingers, family 3], DPH1 [DPH1 homolog (S. cerevisiae)], DPP10 [dipeptidyl-peptidase 10], DPP4 [dipeptidyl-peptidase 4], DPRXP4 [divergent-paired related homeobox pseudogene 4], DPT [dermatopontin], DPYD [dihydropyrimidine dehydrogenase], DPYSL2 [dihydropyrimidinase-like 2], DPYSL3 [dihydropyrimidinase-like 3], DPYSL4 [dihydropyrimidinase-like 4], DPYSL5 [dihydropyrimidinase-like 5], DRD1 [dopamine receptor D1], DRD2 [dopamine receptor D2], DRD3 [dopamine receptor D3], DRD4 [dopamine receptor D4], DRD5 [dopamine receptor D5], DRG1 [developmentally regulated GTP binding protein 1], DRGX [dorsal root ganglia homeobox], DSC2 [desmocollin 2], DSCAM [Down syndrome cell adhesion molecule], DSCAML1 [Down syndrome cell adhesion molecule like 1], DSCR3 [Down syndrome critical region gene 3], DSCR4 [Down syndrome critical region gene 4], DSCR6 [Down syndrome critical region gene 6], DSERG1 [Down syndrome encephalopathy related protein 1], DSG1 [desmoglein 1], DSG2 [desmoglein 2], DSP [desmoplakin], DST [dystonin], DSTN [destrin (actin depolymerizing factor)], DTNBP1 [dystrobrevin binding protein 1], DULLARD [dullard homolog (Xenopus laevis)], DUSP1 [dual specificity phosphatase 1], DUSP13 [dual specificity phosphatase 13], DUSP6 [dual specificity phosphatase 6], DUT [deoxyuridine triphosphatase], DVL1 [dishevelled, dsh homolog 1 (Drosophila)], DYRK1A [dual-specificity tyrosine-(Y)-phosphorylation regulated kinase 1A], DYRK3 [dual-specificity tyrosine-(Y)-phosphorylation regulated kinase 3], DYSF [dysferlin, limb girdle muscular dystrophy 2B (autosomal recessive)], DYX1C1 [dyslexia susceptibility 1 candidate 1], E2F1 [E2F transcription factor 1], EARS2 [glutamyl-tRNA synthetase 2, mitochondrial (putative)], EBF4 [early B-cell factor 4], ECE1 [endothelin converting enzyme 1], ECHS1 [enoyl Coenzyme A hydratase, short chain, 1, mitochondrial], EDN1 [endothelin 1], EDN2 [endothelin 2], EDN3 [endothelin 3], EDNRA [endothelin receptor type A], EDNRB [endothelin receptor type B], EEF1A1 [eukaryotic translation elongation factor 1 alpha 1], EEF2 [eukaryotic translation elongation factor 2], EEF2K [eukaryotic elongation factor-2 kinase], EFHA1 [EF-hand domain family, member A1], EFNA1 [ephrin-A1], EFNA2 [ephrin-A2], EFNA3 [ephrin-A3], EFNA4 [ephrin-A4], EFNA5 [ephrin-A5], EFNB2 [ephrin-B2], EFNB3 [ephrin-B3], EFS [embryonal Fyn-associated substrate], EGF [epidermal growth factor (beta-urogastrone)], EGFR [epidermal growth factor receptor (erythroblastic leukemia viral (v-erb-b) oncogene homolog, avian)], EGLN1 [egl nine homolog 1 (C. elegans)], EGR1 [early growth response 1], EGR2 [early growth response 2], EGR3 [early growth response 3], EHHADH [enoyl-Coenzyme A, hydratase/3-hydroxyacyl Coenzyme A dehydrogenase], EHMT2 [euchromatic histone-lysine N-methyltransferase 2], EID1 [EP300 interacting inhibitor of differentiation 1], EIF1AY [eukaryotic translation initiation factor 1A, Y-linked], EIF2AK2 [eukaryotic translation initiation factor 2-alpha kinase 2], EIF2AK3 [eukaryotic translation initiation factor 2-alpha kinase 3], EIF2B2 [eukaryotic translation initiation factor 2B, subunit 2 beta, 39 kDa], EIF2B5 [eukaryotic translation initiation factor 2B, subunit 5 epsilon, 82 kDa], EIF2S1 [eukaryotic translation initiation factor 2, subunit 1 alpha, 35 kDa], EIF2S2 [eukaryotic translation initiation factor 2, subunit 2 beta, 38 kDa], EIF3M [eukaryotic translation initiation factor 3, subunit M], EIF4E [eukaryotic translation initiation factor 4E], EIF4EBP1 [eukaryotic translation initiation factor 4E binding protein 1], EIF4G1 [eukaryotic translation initiation factor 4 gamma, 1], EIF4H [eukaryotic translation initiation factor 4H], ELANE [elastase, neutrophil expressed], ELAVL1 [ELAV (embryonic lethal, abnormal vision, Drosophila)-like 1 (Hu antigen R)], ELAVL3 [ELAV (embryonic lethal, abnormal vision, Drosophila)-like 3 (Hu antigen C)], ELAVL4 [ELAV (embryonic lethal, abnormal vision, Drosophila)-like 4 (Hu antigen D)], ELF5 [E74-like factor 5 (ets domain transcription factor)], ELK1 [ELK1, member of ETS oncogene family], ELMO1 [engulfment and cell motility 1], ELN [elastin], ELP4 [elongation protein 4 homolog (S. cerevisiae)], EMP2 [epithelial membrane protein 2], EMP3 [epithelial membrane protein 3], EMX1 [empty spiracles homeobox 1], EMX2 [empty spiracles homeobox 2], EN1 [engrailed homeobox 1], EN2 [engrailed homeobox 2], ENAH [enabled homolog (Drosophila)], ENDOG [endonuclease G], ENG [endoglin], ENO1 [enolase 1, (alpha)], ENO2 [enolase 2 (gamma, neuronal)], ENPEP [glutamyl aminopeptidase (aminopeptidase A)], ENPP1 [ectonucleotide pyrophosphatase/phosphodiesterase 1], ENPP2 [ectonucleotide pyrophosphatase/phosphodiesterase 2], ENSA [endosulfine alpha], ENSG00000174496 [ ], ENSG00000183653 [ ], ENSG00000215557 [ ], ENTPD1 [ectonucleoside triphosphate diphosphohydrolase 1], EP300 [E1A binding protein p300], EPCAM [epithelial cell adhesion molecule], EPHA1 [EPH receptor A1], EPHA10 [EPH receptor A10], EPHA2 [EPH receptor A2], EPHA3 [EPH receptor A3], EPHA4 [EPH receptor A4], EPHA5 [EPH receptor A5], EPHA6 [EPH receptor A6], EPHA7 [EPH receptor A7], EPHA8 [EPH receptor A8], EPHB1 [EPH receptor B1], EPHB2 [EPH receptor B2], EPHB3 [EPH receptor B3], EPHB4 [EPH receptor B4], EPHB6 [EPH receptor B6], EPHX2 [epoxide hydrolase 2, cytoplasmic], EPM2A [epilepsy, progressive myoclonus type 2A, Lafora disease (laforin)], EPO [erythropoietin], EPOR [erythropoietin receptor], EPRS [glutamyl-prolyl-tRNA synthetase], EPS15 [epidermal growth factor receptor pathway substrate 15], ERBB2 [v-erb-b2 erythroblastic leukemia viral oncogene homolog 2, neuro/glioblastoma derived oncogene homolog (avian)], ERBB3 [v-erb-b2 erythroblastic leukemia viral oncogene homolog 3 (avian)], ERBB4 [v-erb-a erythroblastic leukemia viral oncogene homolog 4 (avian)], ERC2 [ELKS/RAB6-interacting/CAST family member 2], ERCC2 [excision repair cross-complementing rodent repair deficiency, complementation group 2], ERCC3 [excision repair cross-complementing rodent repair deficiency, complementation group 3 (xeroderma pigmentosum group B complementing)], ERCC5 [excision repair cross-complementing rodent repair deficiency, complementation group 5], ERCC6 [excision repair cross-complementing rodent repair deficiency, complementation group 6], ERCC8 [excision repair cross-complementing rodent repair deficiency, complementation group 8], EREG [epiregulin], ERG [v-ets erythroblastosis virus E26 oncogene homolog (avian)], ERVWE1 [endogenous retroviral family W, env(C7), member 1], ESD [esterase D/formylglutathione hydrolase], ESR1 [estrogen receptor 1], ESR2 [estrogen receptor 2 (ER beta)], ESRRA [estrogen-related receptor alpha], ESRRB [estrogen-related receptor beta], ETS1 [v-ets erythroblastosis virus E26 oncogene homolog 1 (avian)], ETS2 [v-ets erythroblastosis virus E26 oncogene homolog 2 (avian)], ETV1 [ets variant 1], ETV4 [ets variant 4], ETV5 [ets variant 5], ETV6 [ets variant 6], EVL [Enah/Vasp-like], EXOC4 [exocyst complex component 4], EXOC8 [exocyst complex component 8], EXT1 [exostoses (multiple) 1], EXT2 [exostoses (multiple) 2], EZH2 [enhancer of zeste homolog 2 (Drosophila)], EZR [ezrin], F12 [coagulation factor XII (Hageman factor)], F2 [coagulation factor II (thrombin)], F2R [coagulation factor II (thrombin) receptor], F2RL1 [coagulation factor II (thrombin) receptor-like 1], F3 [coagulation factor III (thromboplastin, tissue factor)], F7 [coagulation factor VII (serum prothrombin conversion accelerator)], F8 [coagulation factor VIII, procoagulant component], F9 [coagulation factor IX], FAAH [fatty acid amide hydrolase], FABP3 [fatty acid binding protein 3, muscle and heart (mammary-derived growth inhibitor)], FABP4 [fatty acid binding protein 4, adipocyte], FABP5 [fatty acid binding protein 5 (psoriasis-associated)], FABP7 [fatty acid binding protein 7, brain], FADD [Fas (TNFRSF6)-associated via death domain], FADS2 [fatty acid desaturase 2], FAM120C [family with sequence similarity 120C], FAM165B [family with sequence similarity 165, member B], FAM3C [family with sequence similarity 3, member C], FAM53A [family with sequence similarity 53, member A], FARP2 [FERM, RhoGEF and pleckstrin domain protein 2], FARSA [phenylalanyl-tRNA synthetase, alpha subunit], FAS [Fas (TNF receptor superfamily, member 6)], FASLG [Fas ligand (TNF superfamily, member 6)], FASN [fatty acid synthase], FASTK [Fas-activated serine/threonine kinase], FBLN1 [fibulin 1], FBN1 [fibrillin 1], FBP1 [fructose-1 [6-bisphosphatase 1], FBXO45 [F-box protein 45], FBXW5 [F-box and WD repeat domain containing 5], FBXW7 [F-box and WD repeat domain containing 7], FCER2 [Fc fragment of IgE, low affinity II, receptor for (CD23)], FCGR1A [Fc fragment of IgG, high affinity Ia, receptor (CD64)], FCGR2A [Fc fragment of IgG, low affinity IIa, receptor (CD32)], FCGR2B [Fc fragment of IgG, low affinity IIb, receptor (CD32)], FCGR3A [Fc fragment of IgG, low affinity IIIa, receptor (CD16a)], FCRL3 [Fc receptor-like 3], FDFT1 [farnesyl-diphosphate farnesyltransferase 1], FDX1 [ferredoxin 1], FDXR [ferredoxin reductase], FECH [ferrochelatase (protoporphyria)], FEM1A [fem-1 homolog a (C. elegans)], FER [fer (fps/fes related) tyrosine kinase], FES [feline sarcoma oncogene], FEZ1 [fasciculation and elongation protein zeta 1 (zygin I)], FEZ2 [fasciculation and elongation protein zeta 2 (zygin II)], FEZF1 [FEZ family zinc finger 1], FEZF2 [FEZ family zinc finger 2], FGF1 [fibroblast growth factor 1 (acidic)], FGF19 [fibroblast growth factor 19], FGF2 [fibroblast growth factor 2 (basic)], FGF20 [fibroblast growth factor 20], FGF3 [fibroblast growth factor 3 (murine mammary tumor virus integration site (v-int-2) oncogene homolog)], FGF4 [fibroblast growth factor 4], FGF5 [fibroblast growth factor 5], FGF7 [fibroblast growth factor 7 (keratinocyte growth factor)], FGF8 [fibroblast growth factor 8 (androgen-induced)], FGF9 [fibroblast growth factor 9 (glia-activating factor)], FGFBP1 [fibroblast growth factor binding protein 1], FGFR1 [fibroblast growth factor receptor 1], FGFR2 [fibroblast growth factor receptor 2], FGFR3 [fibroblast growth factor receptor 3], FGFR4 [fibroblast growth factor receptor 4], FHIT [fragile histidine triad gene], FHL1 [four and a half L1M domains 1], FHL2 [four and a half LIM domains 2], FIBP [fibroblast growth factor (acidic) intracellular binding protein], FIGF [c-fos induced growth factor (vascular endothelial growth factor D)], FIGNL1 [fidgetin-like 1], FKBP15 [FK506 binding protein 15, 133 kDa], FKBP1B [FK506 binding protein 1B, 12.6 kDa], FKBP5 [FK506 binding protein 5], FKBP6 [FK506 binding protein 6, 36 kDa], FKBP8 [FK506 binding protein 8, 38 kDa], FKTN [fukutin], FLCN [folliculin], FLG [filaggrin], FLI1 [Friend leukemia virus integration 1], FLNA [filamin A, alpha], FLNB [filamin B, beta], FLNC [filamin C, gamma], FLT1 [fms-related tyrosine kinase 1 (vascular endothelial growth factor/vascular permeability factor receptor)], FLT3 [fms-related tyrosine kinase 3], FMN1 [formin 1], FMNL2 [formin-like 2], FMR1 [fragile X mental retardation 1], FN1 [fibronectin 1], FOLH1 [folate hydrolase (prostate-specific membrane antigen) 1], FOLR1 [folate receptor 1 (adult)], FOS [FBJ murine osteosarcoma viral oncogene homolog], FOSB [FBJ murine osteosarcoma viral oncogene homolog B], FOXC2 [forkhead box C2 (MFH-1, mesenchyme forkhead 1)], FOXG1 [forkhead box G1], FOXL2 [forkhead box L2], FOXM1 [forkhead box M1], FOXO1 [forkhead box 01], FOXO3 [forkhead box O3], FOXP2 [forkhead box P2], FOXP3 [forkhead box P3], FPR1 [formyl peptide receptor 1], FPR2 [formyl peptide receptor 2], FRMD7 [FERM domain containing 7], FRS2 [fibroblast growth factor receptor substrate 2], FRS3 [fibroblast growth factor receptor substrate 3], FRYL [FRY-like], FSCN1 [fascin homolog 1, actin-bundling protein (Strongylocentrotus purpuratus)], FSHB [follicle stimulating hormone, beta polypeptide], FSHR [follicle stimulating hormone receptor], FST [follistatin], FSTL1 [follistatin-like 1], FSTL3 [follistatin-like 3 (secreted glycoprotein)], FTCD [formiminotransferase cyclodeaminase], FTH1 [ferritin, heavy polypeptide 1], FTL [ferritin, light polypeptide], FTMT [ferritin mitochondrial], FTSJ1 [FtsJ homolog 1 (E. coli)], FUCA1 [fucosidase, alpha-L-1, tissue], FURIN [furin (paired basic amino acid cleaving enzyme)], FUT1 [fucosyltransferase 1 (galactoside 2-alpha-L-fucosyltransferase, H blood group)], FUT4 [fucosyltransferase 4 (alpha (1 [3) fucosyltransferase, myeloid-specific)], FXN [frataxin], FXR1 [fragile X mental retardation, autosomal homolog 1], FXR2 [fragile X mental retardation, autosomal homolog 2], FXYD1 [FXYD domain containing ion transport regulator 1], FYB [FYN binding protein (FYB-120/130)], FYN [FYN oncogene related to SRC, FGR, YES], FZD1 [frizzled homolog 1 (Drosophila)], FZD10 [frizzled homolog 10 (Drosophila)], FZD2 [frizzled homolog 2 (Drosophila)], FZD3 [frizzled homolog 3 (Drosophila)], FZD4 [frizzled homolog 4 (Drosophila)], FZD5 [frizzled homolog 5 (Drosophila)], FZD6 [frizzled homolog 6 (Drosophila)], FZD7 [frizzled homolog 7 (Drosophila)], FZD8 [frizzled homolog 8 (Drosophila)], FZD9 [frizzled homolog 9 (Drosophila)], FZR1 [fizzy/cell division cycle 20 related 1 (Drosophila)], G6PD [glucose-6-phosphate dehydrogenase], GAA [glucosidase, alpha; acid], GAB1 [GRB2-associated binding protein 1], GABARAP [GABA(A) receptor-associated protein], GABBR1 [gamma-aminobutyric acid (GABA) B receptor, 1], GABBR2 [gamma-aminobutyric acid (GABA) B receptor, 2], GABPA [GA binding protein transcription factor, alpha subunit 60 kDa], GABRA1 [gamma-aminobutyric acid (GABA) A receptor, alpha 1], GABRA2 [gamma-aminobutyric acid (GABA) A receptor, alpha 2], GABRA3 [gamma-aminobutyric acid (GABA) A receptor, alpha 3], GABRA4 [gamma-aminobutyric acid (GABA) A receptor, alpha 4], GABRA5 [gamma-aminobutyric acid (GABA) A receptor, alpha 5], GABRA6 [gamma-aminobutyric acid (GABA) A receptor, alpha 6], GABRB1 [gamma-aminobutyric acid (GABA) A receptor, beta 1], GABRB2 [gamma-aminobutyric acid (GABA) A receptor, beta 2], GABRB3 [gamma-aminobutyric acid (GABA) A receptor, beta 3], GABRD [gamma-aminobutyric acid (GABA) A receptor, delta], GABRE [gamma-aminobutyric acid (GABA) A receptor, epsilon], GABRG1 [gamma-aminobutyric acid (GABA) A receptor, gamma 1], GABRG2 [gamma-aminobutyric acid (GABA) A receptor, gamma 2], GABRG3 [gamma-aminobutyric acid (GABA) A receptor, gamma 3], GABRP [gamma-aminobutyric acid (GABA) A receptor, pi], GAD1 [glutamate decarboxylase 1 (brain, 67 kDa)], GAD2 [glutamate decarboxylase 2 (pancreatic islets and brain, 65 kDa)], GAL [galanin prepropeptide], GALE [UDP-galactose-4-epimerase], GALK1 [galactokinase 1], GALT [galactose-1-phosphate uridylyltransferase], GAP43 [growth associated protein 43], GAPDH [glyceraldehyde-3-phosphate dehydrogenase], GARS [glycyl-tRNA synthetase], GART [phosphoribosylglycinamide formyltransferase, phosphoribosylglycinamide synthetase, phosphoribosylaminoimidazole synthetase], GAS1 [growth arrest-specific 1], GAS6 [growth arrest-specific 6], GAST [gastrin], GATA1 [GATA binding protein 1 (globin transcription factor 1)], GATA2 [GATA binding protein 2], GATA3 [GATA binding protein 3], GATA4 [GATA binding protein 4], GATA6 [GATA binding protein 6], GBA [glucosidase, beta, acid], GBE1 [glucan (1 [4-alpha-), branching enzyme 1], GBX2 [gastrulation brain homeobox 2], GC [group-specific component (vitamin D binding protein)], GCG [glucagon], GCH1 [GTP cyclohydrolase 1], GCNT1 [glucosaminyl (N-acetyl) transferase 1, core 2], GDAP1 [ganglioside-induced differentiation-associated protein 1], GDF1 [growth differentiation factor 1], GDF11 [growth differentiation factor 11], GDF15 [growth differentiation factor 15], GDF7 [growth differentiation factor 7], GDI1 [GDP dissociation inhibitor 1], GDI2 [GDP dissociation inhibitor 2], GDNF [glial cell derived neurotrophic factor], GDPD5 [glycerophosphodiester phosphodiesterase domain containing 5], GEM [GTP binding protein overexpressed in skeletal muscle], GFAP [glial fibrillary acidic protein], GFER [growth factor, augmenter of liver regeneration], GFI1B [growth factor independent 1B transcription repressor], GFRA1 [GDNF family receptor alpha 1], GFRA2 [GDNF family receptor alpha 2], GFRA3 [GDNF family receptor alpha 3], GFRA4 [GDNF family receptor alpha 4], GGCX [gamma-glutamyl carboxylase], GGNBP2 [gametogenetin binding protein 2], GGT1 [gamma-glutamyltransferase 1], GGT2 [gamma-glutamyltransferase 2], GH1 [growth hormone 1], GHR [growth hormone receptor], GHRH [growth hormone releasing hormone], GHRHR [growth hormone releasing hormone receptor], GHRL [ghrelin/obestatin prepropeptide], GHSR [growth hormone secretagogue receptor], GIPR [gastric inhibitory polypeptide receptor], GIT1 [G protein-coupled receptor kinase interacting ArfGAP 1], GJA1 [gap junction protein, alpha 1, 43 kDa], GJA4 [gap junction protein, alpha 4, 37 kDa], GJA5 [gap junction protein, alpha 5, 40 kDa], GJB1 [gap junction protein, beta 1, 32 kDa], GJB2 [gap junction protein, beta 2, 26 kDa], GJB6 [gap junction protein, beta 6, kDa], GLA [galactosidase, alpha], GLB1 [galactosidase, beta 1], GLDC [glycine dehydrogenase (decarboxylating)], GLI1 [GLI family zinc finger 1], GLI2 [GLI family zinc finger 2], GLI3 [GLI family zinc finger 3], GLIS1 [GLIS family zinc finger 1], GLIS2 [GLIS family zinc finger 2], GLO1 [glyoxalase I], GLRA2 [glycine receptor, alpha 2], GLRB [glycine receptor, beta], GLS [glutaminase], GLUD1 [glutamate dehydrogenase 1], GLUD2 [glutamate dehydrogenase 2], GLUL [glutamate-ammonia ligase (glutamine synthetase)], GLYAT [glycine-N-acyltransferase], GMFB [glia maturation factor, beta], GMNN [geminin, DNA replication inhibitor], GMPS [guanine monophosphate synthetase], GNA11 [guanine nucleotide binding protein (G protein), alpha 11 (Gq class)], GNA12 [guanine nucleotide binding protein (G protein) alpha 12], GNA13 [guanine nucleotide binding protein (G protein), alpha 13], GNA14 [guanine nucleotide binding protein (G protein), alpha 14], GNA15 [guanine nucleotide binding protein (G protein), alpha 15 (Gq class)], GNAI1 [guanine nucleotide binding protein (G protein), alpha inhibiting activity polypeptide 1], GNAI2 [guanine nucleotide binding protein (G protein), alpha inhibiting activity polypeptide 2], GNAI3 [guanine nucleotide binding protein (G protein), alpha inhibiting activity polypeptide 3], GNAL [guanine nucleotide binding protein (G protein), alpha activating activity polypeptide, olfactory type], GNAO1 [guanine nucleotide binding protein (G protein), alpha activating activity polypeptide 0], GNAQ [guanine nucleotide binding protein (G protein), q polypeptide], GNAS [GNAS complex locus], GNAT1 [guanine nucleotide binding protein (G protein), alpha transducing activity polypeptide 1], GNAT2 [guanine nucleotide binding protein (G protein), alpha transducing activity polypeptide 2], GNAZ [guanine nucleotide binding protein (G protein), alpha z polypeptide], GNB1 [guanine nucleotide binding protein (G protein), beta polypeptide 1], GNB1L [guanine nucleotide binding protein (G protein), beta polypeptide 1-like], GNB2 [guanine nucleotide binding protein (G protein), beta polypeptide 2], GNB2L1 [guanine nucleotide binding protein (G protein), beta polypeptide 2-like 1], GNB3 [guanine nucleotide binding protein (G protein), beta polypeptide 3], GNB4 [guanine nucleotide binding protein (G protein), beta polypeptide 4], GNB5 [guanine nucleotide binding protein (G protein), beta 5], GNG10 [guanine nucleotide binding protein (G protein), gamma 10], GNG11 [guanine nucleotide binding protein (G protein), gamma 11], GNG12 [guanine nucleotide binding protein (G protein), gamma 12], GNG13 [guanine nucleotide binding protein (G protein), gamma 13], GNG2 [guanine nucleotide binding protein (G protein), gamma 2], GNG3 [guanine nucleotide binding protein (G protein), gamma 3], GNG4 [guanine nucleotide binding protein (G protein), gamma 4], GNG5 [guanine nucleotide binding protein (G protein), gamma 5], GNG7 [guanine nucleotide binding protein (G protein), gamma 7], GNLY [granulysin], GNRH1 [gonadotropin-releasing hormone 1 (luteinizing-releasing hormone)], GNRHR [gonadotropin-releasing hormone receptor], GOLGA2 [golgin A2], GOLGA4 [golgin A4], GOT2 [glutamic-oxaloacetic transaminase 2, mitochondrial (aspartate aminotransferase 2)], GP1BA [glycoprotein Ib (platelet), alpha polypeptide], GP5 [glycoprotein V (platelet)], GP6 [glycoprotein VI (platelet)], GP9 [glycoprotein IX (platelet)], GPC1 [glypican 1], GPC3 [glypican 3], GPD1 [glycerol-3-phosphate dehydrogenase 1 (soluble)], GPHN [gephyrin], GPI [glucose phosphate isomerase], GPM6A [glycoprotein M6A], GPM6B [glycoprotein M6B], GPR161 [G protein-coupled receptor 161], GPR182 [G protein-coupled receptor 182], GPR56 [G protein-coupled receptor 56], GPRC6A [G protein-coupled receptor, family C, group 6, member A], GPRIN1 [G protein regulated inducer of neurite outgrowth 1], GPT [glutamic-pyruvate transaminase (alanine aminotransferase)], GPT2 [glutamic pyruvate transaminase (alanine aminotransferase) 2], GPX1 [glutathione peroxidase 1], GPX3 [glutathione peroxidase 3 (plasma)], GPX4 [glutathione peroxidase 4 (phospholipid hydroperoxidase)], GRAP [GRB2-related adaptor protein], GRB10 [growth factor receptor-bound protein 10], GRB2 [growth factor receptor-bound protein 2], GRB7 [growth factor receptor-bound protein 7], GREM1 [gremlin 1, cysteine knot superfamily, homolog (Xenopus laevis)], GRIA1 [glutamate receptor, ionotropic, AMPA 1], GRIA2 [glutamate receptor, ionotropic, AMPA 2], GRIA3 [glutamate receptor, ionotrophic, AMPA 3], GRID2 [glutamate receptor, ionotropic, delta 2], GRID21P [glutamate receptor, ionotropic, delta 2 (Grid2) interacting protein], GRIK1 [glutamate receptor, ionotropic, kainate 1], GRIK2 [glutamate receptor, ionotropic, kainate 2], GRIN1 [glutamate receptor, ionotropic, N-methyl D-aspartate 1], GRIN2A [glutamate receptor, ionotropic, N-methyl D-aspartate 2A], GRIP1 [glutamate receptor interacting protein 1], GRLF1 [glucocorticoid receptor DNA binding factor 1], GRM1 [glutamate receptor, metabotropic 1], GRM2 [glutamate receptor, metabotropic 2], GRM5 [glutamate receptor, metabotropic 5], GRM7 [glutamate receptor, metabotropic 7], GRM8 [glutamate receptor, metabotropic 8], GRN [granulin], GRP [gastrin-releasing peptide], GRPR [gastrin-releasing peptide receptor], GSK3B [glycogen synthase kinase 3 beta], GSN [gelsolin], GSR [glutathione reductase], GSS [glutathione synthetase], GSTA1 [glutathione S-transferase alpha 1], GSTM1 [glutathione S-transferase mu 1], GSTP1 [glutathione S-transferase pi 1], GSTT1 [glutathione S-transferase theta 1], GSTZ1 [glutathione transferase zeta 1], GTF2B [general transcription factor IIB], GTF2E2 [general transcription factor IIE, polypeptide 2, beta 34 kDa], GTF2H1 [general transcription factor IIH, polypeptide 1, 62 kDa], GTF2H2 [general transcription factor IIH, polypeptide 2, 44 kDa], GTF2H3 [general transcription factor IIH, polypeptide 3, 34 kDa], GTF2H4 [general transcription factor IIH, polypeptide 4, 52 kDa], GTF2I [general transcription factor IIi], GTF2IRD1 [GTF2I repeat domain containing 1], GTF2IRD2 [GTF2I repeat domain containing 2], GUCA2A [guanylate cyclase activator 2A (guanylin)], GUCY1A3 [guanylate cyclase 1, soluble, alpha 3], GUSB [glucuronidase, beta], GYPA [glycophorin A (MNS blood group)], GYPC [glycophorin C (Gerbich blood group)], GZF1 [GDNF-inducible zinc finger protein 1], GZMA [granzyme A (granzyme 1, cytotoxic T-lymphocyte-associated serine esterase 3)], GZMB [granzyme B (granzyme 2, cytotoxic T-lymphocyte-associated serine esterase 1)], H19 [H19, imprinted maternally expressed transcript (non-protein coding)], H1F0 [H1 histone family, member 0], H2AFX [H2A histone family, member X], H2AFY [H2A histone family, member Y], H6PD [hexose-6-phosphate dehydrogenase (glucose 1-dehydrogenase)], HADHA [hydroxyacyl-Coenzyme A dehydrogenase/3-ketoacyl-Coenzyme A thiolase/enoyl-Coenzyme A hydratase (trifunctional protein), alpha subunit], HAMP [hepcidin antimicrobial peptide], HAND1 [heart and neural crest derivatives expressed 1], HAND2 [heart and neural crest derivatives expressed 2], HAP1 [huntingtin-associated protein 1], HAPLN1 [hyaluronan and proteoglycan link protein 1], HARS [histidyl-tRNA synthetase], HAS1 [hyaluronan synthase 1], HAS2 [hyaluronan synthase 2], HAS3 [hyaluronan synthase 3], HAX1 [HCLS1 associated protein X-1], HBA2 [hemoglobin, alpha 2], HBB [hemoglobin, beta], HBEGF [heparin-binding EGF-like growth factor], HBG1 [hemoglobin, gamma A], HBG2 [hemoglobin, gamma G], HCCS [holocytochrome c synthase (cytochrome c heme-lyase)], HCK [hemopoietic cell kinase], HCLS1 [hematopoietic cell-specific Lyn substrate 1], HCN4 [hyperpolarization activated cyclic nucleotide-gated potassium channel 4], HCRT [hypocretin (orexin) neuropeptide precursor], HCRTR1 [hypocretin (orexin) receptor 1], HCRTR2 [hypocretin (orexin) receptor 2], HDAC1 [histone deacetylase 1], HDAC2 [histone deacetylase 2], HDAC4 [histone deacetylase 4], HDAC9 [histone deacetylase 9], HDC [histidine decarboxylase], HDLBP [high density lipoprotein binding protein], HEPACAM [hepatocyte cell adhesion molecule], HES1 [hairy and enhancer of split 1, (Drosophila)], HES3 [hairy and enhancer of split 3 (Drosophila)], HES5 [hairy and enhancer of split 5 (Drosophila)], HES6 [hairy and enhancer of split 6 (Drosophila)], HEXA [hexosaminidase A (alpha polypeptide)], HFE [hemochromatosis], HFE2 [hemochromatosis type 2 (juvenile)], HGF [hepatocyte growth factor (hepapoietin A; scatter factor)], HGS [hepatocyte growth factor-regulated tyrosine kinase substrate], HHEX [hematopoietically expressed homeobox], HHIP [hedgehog interacting protein], HIF1A [hypoxia inducible factor 1, alpha subunit (basic helix-loop-helix transcription factor)], HINT1 [histidine triad nucleotide binding protein 1], HIPK2 [homeodomain interacting protein kinase 2], HIRA [HIR histone cell cycle regulation defective homolog A (S. cerevisiae)], HIRIP3 [HIRA interacting protein 3], H1ST1H2AB [histone cluster 1, H2ab], H1ST1H2AC [histone cluster 1, H2ac], H1ST1H2AD [histone cluster 1, H2ad], H1ST1H2AE [histone cluster 1, H2ae], H1ST1H2AG [histone cluster 1, H2ag], H1ST1H2A1 [histone cluster 1, H2ai], H1ST1H2AJ [histone cluster 1, H2aj], H1ST1H2AK [histone cluster 1, H2ak], H1ST1H2AL [histone cluster 1, H2al], H1ST1H2AM [histone cluster 1, H2 am], HIST1H3E [histone cluster 1, H3e], H1ST2H2AA3 [histone cluster 2, H2aa3], H1ST2H2AA4 [histone cluster 2, H2aa4], H1ST2H2AC [histone cluster 2, H2ac], HKR1 [GLI-Kruppel family member HKR1], HLA-A [major histocompatibility complex, class I, A], HLA-B [major histocompatibility complex, class I, B], HLA-C [major histocompatibility complex, class I, C], HLA-DMA [major histocompatibility complex, class II, DM alpha], HLA-DOB [major histocompatibility complex, class II, DO beta], HLA-DQA1 [major histocompatibility complex, class II, DQ alpha 1], HLA-DQB1 [major histocompatibility complex, class II, DQ beta 1], HLA-DRA [major histocompatibility complex, class II, DR alpha], HLA-DRB1 [major histocompatibility complex, class II, DR beta 1], HLA-DRB4 [major histocompatibility complex, class II, DR beta 4], HLA-DRB5 [major histocompatibility complex, class II, DR beta 5], HLA-E [major histocompatibility complex, class I, E], HLA-F [major histocompatibility complex, class I, F], HLA-G [major histocompatibility complex, class I, G], HLCS [holocarboxylase synthetase (biotin-(proprionyl-Coenzyme A-carboxylase (ATP-hydrolysing)) ligase)], HMBS [hydroxymethylbilane synthase], HMGA1 [high mobility group AT-hook 1], HMGA2 [high mobility group AT-hook 2], HMGB1 [high-mobility group box 1], HMGCR [3-hydroxy-3-methylglutaryl-Coenzyme A reductase], HMGN1 [high-mobility group nucleosome binding domain 1], HMOX1 [heme oxygenase (decycling) 1], HMOX2 [heme oxygenase (decycling) 2], HNF1A [HNF1 homeobox A], HNF4A [hepatocyte nuclear factor 4, alpha], HNMT [histamine N-methyltransferase], HNRNPA2B1 [heterogeneous nuclear ribonucleoprotein A2/B1], HNRNPK [heterogeneous nuclear ribonucleoprotein K], HNRNPL [heterogeneous nuclear ribonucleoprotein L], HNRNPU [heterogeneous nuclear ribonucleoprotein U (scaffold attachment factor A)], HNRPDL [heterogeneous nuclear ribonucleoprotein D-like], HOMER1 [homer homolog 1 (Drosophila)], HOXA1 [homeobox A1], HOXA10 [homeobox A10], HOXA2 [homeobox A2], HOXA5 [homeobox A5], HOXA9 [homeobox A9], HOXB1 [homeobox B1], HOXB4 [homeobox B4], HOXB9 [homeobox B9], HOXD11 [homeobox D11], HOXD12 [homeobox D12], HOXD13 [homeobox D13], HP [haptoglobin], HPD [4-hydroxyphenylpyruvate dioxygenase], HPRT1 [hypoxanthine phosphoribosyltransferase 1], HPS4 [Hermansky-Pudlak syndrome 4], HPX [hemopexin], HRAS [v-Ha-ras Harvey rat sarcoma viral oncogene homolog], HRG [histidine-rich glycoprotein], HRH1 [histamine receptor H1], HRH2 [histamine receptor H2], HRH3 [histamine receptor H3], HSD11B1 [hydroxysteroid (11-beta) dehydrogenase 1], HSD11B2 [hydroxysteroid (11-beta) dehydrogenase 2], HSD17B10 [hydroxysteroid (17-beta) dehydrogenase 10], HSD3B2 [hydroxy-delta-5-steroid dehydrogenase, 3 beta- and steroid delta-isomerase 2], HSF1 [heat shock transcription factor 1], HSP90AA1 [heat shock protein 90 kDa alpha (cytosolic), class A member 1], HSP90B1 [heat shock protein 90 kDa beta (Grp94), member 1], HSPA1A [heat shock 70 kDa protein 1A], HSPA4 [heat shock 70 kDa protein 4], HSPA5 [heat shock 70 kDa protein 5 (glucose-regulated protein, 78 kDa)], HSPA8 [heat shock 70 kDa protein 8], HSPA9 [heat shock 70 kDa protein 9 (mortalin)], HSPB1 [heat shock 27 kDa protein 1], HSPD1 [heat shock 60 kDa protein 1 (chaperonin)], HSPE1 [heat shock 10 kDa protein 1 (chaperonin 10)], HSPG2 [heparan sulfate proteoglycan 2], HTN1 [histatin 1], HTR1A [5-hydroxytryptamine (serotonin) receptor 1A], HTR1B [5-hydroxytryptamine (serotonin) receptor 1B], HTR1D [5-hydroxytryptamine (serotonin) receptor 1D], HTR1E [5-hydroxytryptamine (serotonin) receptor 1E], HTR1F [5-hydroxytryptamine (serotonin) receptor 1F], HTR2A [5-hydroxytryptamine (serotonin) receptor 2A], HTR2B [5-hydroxytryptamine (serotonin) receptor 2B], HTR2c [5-hydroxytryptamine (serotonin) receptor 20], HTR3A [5-hydroxytryptamine (serotonin) receptor 3A], HTR3B [5-hydroxytryptamine (serotonin) receptor 3B], HTR5A [5-hydroxytryptamine (serotonin) receptor 5A], HTR6 [5-hydroxytryptamine (serotonin) receptor 6], HTR7 [5-hydroxytryptamine (serotonin) receptor 7 (adenylate cyclase-coupled)], HTT [huntingtin], HYAL1 [hyaluronoglucosaminidase 1], HYOU1 [hypoxia up-regulated 1], IAPP [islet amyloid polypeptide], IBSP [integrin-binding sialoprotein], ICAM1 [intercellular adhesion molecule 1], ICAM2 [intercellular adhesion molecule 2], ICAM3 [intercellular adhesion molecule 3], ICAM5 [intercellular adhesion molecule 5, telencephalin], ICOS [inducible T-cell co-stimulator], ID1 [inhibitor of DNA binding 1, dominant negative helix-loop-helix protein], ID2 [inhibitor of DNA binding 2, dominant negative helix-loop-helix protein], ID3 [inhibitor of DNA binding 3, dominant negative helix-loop-helix protein], ID4 [inhibitor of DNA binding 4, dominant negative helix-loop-helix protein], IDE [insulin-degrading enzyme], IDI1 [isopentenyl-diphosphate delta isomerase 1], IDO1 [indoleamine 2 [3-dioxygenase 1], IDS [iduronate 2-sulfatase], IDUA [iduronidase, alpha-L-], IER3 [immediate early response 3], IF127 [interferon, alpha-inducible protein 27], IFNα1 [interferon, alpha 1], IFNα2 [interferon, alpha 2], IFNAR1 [interferon (alpha, beta and omega) receptor 1], IFNAR2 [interferon (alpha, beta and omega) receptor 2], IFNB1 [interferon, beta 1, fibroblast], IFNG [interferon, gamma], IFNGR1 [interferon gamma receptor 1], IFNGR2 [interferon gamma receptor 2 (interferon gamma transducer 1)], IGF1 [insulin-like growth factor 1 (somatomedin C)], IGF1R [insulin-like growth factor 1 receptor], IGF2 [insulin-like growth factor 2 (somatomedin A)], IGF2R [insulin-like growth factor 2 receptor], IGFBP1 [insulin-like growth factor binding protein 1], IGFBP2 [insulin-like growth factor binding protein 2, 36 kDa], IGFBP3 [insulin-like growth factor binding protein 3], IGFBP4 [insulin-like growth factor binding protein 4], IGFBP5 [insulin-like growth factor binding protein 5], IGFBP6 [insulin-like growth factor binding protein 6], IGFBP7 [insulin-like growth factor binding protein 7], IGHA1 [immunoglobulin heavy constant alpha 1], IGHE [immunoglobulin heavy constant epsilon], IGHG1 [immunoglobulin heavy constant gamma 1 (G1m marker)], IGHJ1 [immunoglobulin heavy joining 1], IGHM [immunoglobulin heavy constant mu], IGHMBP2 [immunoglobulin mu binding protein 2], IGKC [immunoglobulin kappa constant], IKBKAP [inhibitor of kappa light polypeptide gene enhancer in B-cells, kinase complex-associated protein], IKBKB [inhibitor of kappa light polypeptide gene enhancer in B-cells, kinase beta], IKZF1 [IKAROS family zinc finger 1 (Ikaros)], IL10 [interleukin 10], IL10RA [interleukin 10 receptor, alpha], IL10RB [interleukin 10 receptor, beta], IL11 [interleukin 11], IL11RA [interleukin 11 receptor, alpha], IL12A [interleukin 12A (natural killer cell stimulatory factor 1, cytotoxic lymphocyte maturation factor 1, p35)], IL12B [interleukin 12B (natural killer cell stimulatory factor 2, cytotoxic lymphocyte maturation factor 2, p40)], IL12RB1 [interleukin 12 receptor, beta 1], IL13 [interleukin 13], IL15 [interleukin 15], IL15RA [interleukin 15 receptor, alpha], IL16 [interleukin 16 (lymphocyte chemoattractant factor)], IL17A [interleukin 17A], IL18 [interleukin 18 (interferon-gamma-inducing factor)], IL18BP [interleukin 18 binding protein], ILIA [interleukin 1, alpha], IL1B [interleukin 1, beta], IL1F7 [interleukin 1 family, member 7 (zeta)], IL1R1 [interleukin 1 receptor, type I], IL1R2 [interleukin 1 receptor, type II], IL1RAPL1 [interleukin 1 receptor accessory protein-like 1], IL1RL1 [interleukin 1 receptor-like 1], IL1RN [interleukin 1 receptor antagonist], IL2 [interleukin 2], IL21 [interleukin 21], IL22 [interleukin 22], IL23A [interleukin 23, alpha subunit p19], IL23R [interleukin 23 receptor], IL29 [interleukin 29 (interferon, lambda 1)], IL2RA [interleukin 2 receptor, alpha], IL2RB [interleukin 2 receptor, beta], IL3 [interleukin 3 (colony-stimulating factor, multiple)], IL3RA [interleukin 3 receptor, alpha (low affinity)], IL4 [interleukin 4], IL4R [interleukin 4 receptor], IL5 [interleukin 5 (colony-stimulating factor, eosinophil)], IL6 [interleukin 6 (interferon, beta 2)], IL6R [interleukin 6 receptor], IL6ST [interleukin 6 signal transducer (gp130, oncostatin M receptor)], IL7 [interleukin 7], IL7R [interleukin 7 receptor], IL8 [interleukin 8], IL9 [interleukin 9], ILK [integrin-linked kinase], IMMP2L [IMP2 inner mitochondrial membrane peptidase-like (S. cerevisiae)], IMMT [inner membrane protein, mitochondrial (mitofilin)], IMPA1 [inositol(myo)-1(or 4)-monophosphatase 1], IMPDH2 [IMP (inosine monophosphate) dehydrogenase 2], INADL [InaD-like (Drosophila)], INCENP [inner centromere protein antigens 135/155 kDa], ING1 [inhibitor of growth family, member 1], ING3 [inhibitor of growth family, member 3], INHA [inhibin, alpha], INHBA [inhibin, beta A], INPP1 [inositol polyphosphate-1-phosphatase], INPP5D [inositol polyphosphate-5-phosphatase, 145 kDa], INPP5E [inositol polyphosphate-5-phosphatase, 72 kDa], INPP5J [inositol polyphosphate-5-phosphatase J], INPPL1 [inositol polyphosphate phosphatase-like 1], INS [insulin], INSIG2 [insulin induced gene 2], INS-IGF2 [INS-IGF2 readthrough transcript], INSL3 [insulin-like 3 (Leydig cell)], INSR [insulin receptor], INVS [inversin], IQCB1 [IQ motif containing B1], IQGAP1 [IQ motif containing GTPase activating protein 1], IRAK1 [interleukin-1 receptor-associated kinase 1], IRAK4 [interleukin-1 receptor-associated kinase 4], IREB2 [iron-responsive element binding protein 2], IRF1 [interferon regulatory factor 1], IRF4 [interferon regulatory factor 4], IRF8 [interferon regulatory factor 8], IRS1 [insulin receptor substrate 1], IRS2 [insulin receptor substrate 2], IRS4 [insulin receptor substrate 4], IRX3 [iroquois homeobox 3], ISG15 [ISG15 ubiquitin-like modifier], ISL1 [ISL L1M homeobox 1], ISL2 [ISL LIM homeobox 2], ISLR2 [immunoglobulin superfamily containing leucine-rich repeat 2], ITGA2 [integrin, alpha 2 (CD49B, alpha 2 subunit of VLA-2 receptor)], ITGA2B [integrin, alpha 2b (platelet glycoprotein IIb of IIb/IIIa complex, antigen CD41)], ITGA3 [integrin, alpha 3 (antigen CD49C, alpha 3 subunit of VLA-3 receptor)], ITGA4 [integrin, alpha 4 (antigen CD49D, alpha 4 subunit of VLA-4 receptor)], ITGA5 [integrin, alpha 5 (fibronectin receptor, alpha polypeptide)], ITGA6 [integrin, alpha 6], ITGA9 [integrin, alpha 9], ITGAL [integrin, alpha L (antigen CD11A (p180), lymphocyte function-associated antigen 1; alpha polypeptide)], ITGAM [integrin, alpha M (complement component 3 receptor 3 subunit)], ITGAV [integrin, alpha V (vitronectin receptor, alpha polypeptide, antigen CD51)], ITGAX [integrin, alpha X (complement component 3 receptor 4 subunit)], ITGB1 [integrin, beta 1 (fibronectin receptor, beta polypeptide, antigen CD29 includes MDF2, MSK12)], ITGB2 [integrin, beta 2 (complement component 3 receptor 3 and 4 subunit)], ITGB3 [integrin, beta 3 (platelet glycoprotein IIIa, antigen CD61)], ITGB4 [integrin, beta 4], ITGB6 [integrin, beta 6], ITGB7 [integrin, beta 7], ITIH4 [inter-alpha (globulin) inhibitor H4 (plasma Kallikrein-sensitive glycoprotein)], ITM2B [integral membrane protein 2B], ITPR1 [inositol 1 [4 [5-triphosphate receptor, type 1], ITPR2 [inositol 1 [4 [5-triphosphate receptor, type 2], ITPR3 [inositol 1 [4 [5-triphosphate receptor, type 3], ITSN1 [intersectin 1 (SH3 domain protein)], ITSN2 [intersectin 2], IVL [involucrin], JAG1 bagged 1 (Alagille syndrome)], JAK1 [Janus kinase 1], JAK2 [Janus kinase 2], JAK3 [Janus kinase 3], JAM2 [junctional adhesion molecule 2], JARID2 [jumonji, AT rich interactive domain 2], JMJD1C [jumonji domain containing 10], JMY [junction mediating and regulatory protein, p53 cofactor], JRKL [jerky homolog-like (mouse)], JUN [jun oncogene], JUNB [jun B proto-oncogene], JUND [jun D proto-oncogene], JUP [junction plakoglobin], KAL1 [Kallmann syndrome 1 sequence], KALRN [kalirin, RhoGEF kinase], KARS [lysyl-tRNA synthetase], KAT2B [K(lysine) acetyltransferase 2B], KATNA1 [katanin p60 (ATPase-containing) subunit A 1], KATNB1 [katanin p80 (WD repeat containing) subunit B1], KCNA4 [potassium voltage-gated channel, shaker-related subfamily, member 4], KCND1 [potassium voltage-gated channel, Shal-related subfamily, member 1], KCND2 [potassium voltage-gated channel, Shal-related subfamily, member 2], KCNE1 [potassium voltage-gated channel, Isk-related family, member 1], KCNE2 [potassium voltage-gated channel, Isk-related family, member 2], KCNH2 [potassium voltage-gated channel, subfamily H (eag-related), member 2], KCNH4 [potassium voltage-gated channel, subfamily H (eag-related), member 4], KCNJ15 [potassium inwardly-rectifying channel, subfamily J, member 15], KCNJ3 [potassium inwardly-rectifying channel, subfamily J, member 3], KCNJ4 [potassium inwardly-rectifying channel, subfamily J, member 4], KCNJ5 [potassium inwardly-rectifying channel, subfamily J, member 5], KCNJ6 [potassium inwardly-rectifying channel, subfamily J, member 6], KCNMA1 [potassium large conductance calcium-activated channel, subfamily M, alpha member 1], KCNN1 [potassium intermediate/small conductance calcium-activated channel, subfamily N, member 1], KCNN2 [potassium intermediate/small conductance calcium-activated channel, subfamily N, member 2], KCNN3 [potassium intermediate/small conductance calcium-activated channel, subfamily N, member 3], KCNQ1 [potassium voltage-gated channel, KQT-like subfamily, member 1], KCNQ2 [potassium voltage-gated channel, KQT-like subfamily, member 2], KDM5C [lysine (K)-specific demethylase 5C], KDR [kinase insert domain receptor (a type III receptor tyrosine kinase)], KIAA0101 [KIAA0101], KIAA0319 [KIAA0319], KIAA1715 [KIAA1715], KIDINS220 [kinase D-interacting substrate, 220 kDa], KIF15 [kinesin family member 15], KIF16B [kinesin family member 16B], KIF1A [kinesin family member 1A], KIF2A [kinesin heavy chain member 2A], KIF2B [kinesin family member 2B], KIF3A [kinesin family member 3A], KIF5C [kinesin family member 5C], KIF7 [kinesin family member 7], KIR2DL1 [killer cell immunoglobulin-like receptor, two domains, long cytoplasmic tail, 1], KIR2DL3 [killer cell immunoglobulin-like receptor, two domains, long cytoplasmic tail, 3], KIR2DS2 [killer cell immunoglobulin-like receptor, two domains, short cytoplasmic tail, 2], KIR3DL1 [killer cell immunoglobulin-like receptor, three domains, long cytoplasmic tail, 1], KIR3DL2 [killer cell immunoglobulin-like receptor, three domains, long cytoplasmic tail, 2], KIRREL3 [kin of IRRE like 3 (Drosophila)], KISS1 [KiSS-1 metastasis-suppressor], KISS1R [KISS1 receptor], KIT [v-kit Hardy-Zuckerman 4 feline sarcoma viral oncogene homolog], KITLG [KIT ligand], KL [klotho], KLF7 [Kruppel-like factor 7 (ubiquitous)], KLK1 [kallikrein 1], KLK10 [kallikrein-related peptidase 10], KLK11 [kallikrein-related peptidase 11], KLK2 [kallikrein-related peptidase 2], KLK3 [kallikrein-related peptidase 3], KLK5 [kallikrein-related peptidase 5], KLRD1 [killer cell lectin-like receptor subfamily D, member 1], KLRK1 [killer cell lectin-like receptor subfamily K, member 1], KMO [kynurenine 3-monooxygenase (kynurenine 3-hydroxylase)], KNG1 [kininogen 1], KPNA2 [karyopherin alpha 2 (RAG cohort 1, importin alpha 1)], KPNB1 [karyopherin (importin) beta 1], KPTN [kaptin (actin binding protein)], KRAS [v-Ki-ras2 Kirsten rat sarcoma viral oncogene homolog], KRIT1 [KRIT1, ankyrin repeat containing], KRT1 [keratin 1], KRT10 [keratin 10], KRT14 [keratin 14], KRT18 [keratin 18], KRT19 [keratin 19], KRT3 [keratin 3], KRT5 [keratin 5], KRT7 [keratin 7], KRT8 [keratin 8], KRTAP19-3 [keratin associated protein 19-3], KRTAP2-1 [keratin associated protein 2-1], L1CAM [L1 cell adhesion molecule], LACTB [lactamase, beta], LALBA [lactalbumin, alpha-], LAMA1 [laminin, alpha 1], LAMB1 [laminin, beta 1], LAMB2 [laminin, beta 2 (laminin S)], LAMB4 [laminin, beta 4], LAMP1 [lysosomal-associated membrane protein 1], LAMP2 [lysosomal-associated membrane protein 2], LAP3 [leucine aminopeptidase 3], LAPTM4A [lysosomal protein transmembrane 4 alpha], LARGE [like-glycosyltransferase], LARS [leucyl-tRNA synthetase], LASP1 [LIM and SH3 protein 1], LAT2 [linker for activation of T cells family, member 2], LBP [lipopolysaccharide binding protein], LBR [lamin B receptor], LCA10 [lung carcinoma-associated protein 10], LCA5 [Leber congenital amaurosis 5], LCAT [lecithin-cholesterol acyltransferase], LCK [lymphocyte-specific protein tyrosine kinase], LCN1 [lipocalin 1 (tear prealbumin)], LCN2 [lipocalin 2], LCP1 [lymphocyte cytosolic protein 1 (L-plastin)], LCP2 [lymphocyte cytosolic protein 2 (SH2 domain containing leukocyte protein of 76 kDa)], LCT [lactase], LDB1 [LIM domain binding 1], LDB2 [LIM domain binding 2], LDHA [lactate dehydrogenase A], LDLR [low density lipoprotein receptor], LDLRAP1 [low density lipoprotein receptor adaptor protein 1], LEF1 [lymphoid enhancer-binding factor 1], LEO1 [Leo1, Paf1/RNA polymerase II complex component, homolog (S. cerevisiae)], LEP [leptin], LEPR [leptin receptor], LGALS13 [lectin, galactoside-binding, soluble, 13], LGALS3 [lectin, galactoside-binding, soluble, 3], LGMN [legumain], LGR4 [leucine-rich repeat-containing G protein-coupled receptor 4], LGTN [ligatin], LHCGR [luteinizing hormone/choriogonadotropin receptor], LHFPL3 [lipoma HMGIC fusion partner-like 3], LHX1 [LIM homeobox 1], LHX2 [LIM homeobox 2], LHX3 [LIM homeobox 3], LHX4 [LIM homeobox 4], LHX9 [LIM homeobox 9], LIF [leukemia inhibitory factor (cholinergic differentiation factor)], LIFR [leukemia inhibitory factor receptor alpha], LIG1 [ligase I, DNA, ATP-dependent], LIG3 [ligase III, DNA, ATP-dependent], LIG4 [ligase IV, DNA, ATP-dependent], LILRA3 [leukocyte immunoglobulin-like receptor, subfamily A (without TM domain), member 3], LILRB1 [leukocyte immunoglobulin-like receptor, subfamily B (with TM and ITIM domains), member 1], LIMK1 [LIM domain kinase 1], LIMK2 [LIM domain kinase 2], LIN7A [lin-7 homolog A (C. elegans)], LIN7B [lin-7 homolog B (C. elegans)], LIN7C [lin-7 homolog C (C. elegans)], LINGO1 [leucine rich repeat and Ig domain containing 1], LIPC [lipase, hepatic], LIPE [lipase, hormone-sensitive], LLGL1 [lethal giant larvae homolog 1 (Drosophila)], LMAN1 [lectin, mannose-binding, 1], LMNA [lamin A/C], LMO2 [LIM domain only 2 (rhombotin-like 1)], LMX1A [LIM homeobox transcription factor 1, alpha], LMX1B [LIM homeobox transcription factor 1, beta], LNPEP [leucyl/cystinyl aminopeptidase], LOC400590 [hypothetical LOC400590], LOC646021 [similar to hCG1774990], LOC646030 [similar to hCG1991475], LOC646627 [phospholipase inhibitor], LOR [loricrin], LOX [lysyl oxidase], LOXL1 [lysyl oxidase-like 1], LPA [lipoprotein, Lp(a)], LPL [lipoprotein lipase], LPO [lactoperoxidase], LPP [LIM domain containing preferred translocation partner in lipoma], LPPR1 [lipid phosphate phosphatase-related protein type 1], LPPR3 [lipid phosphate phosphatase-related protein type 3], LPPR4 [lipid phosphate phosphatase-related protein type 4], LPXN [leupaxin], LRP1 [low density lipoprotein receptor-related protein 1], LRP6 [low density lipoprotein receptor-related protein 6], LRP8 [low density lipoprotein receptor-related protein 8, apolipoprotein e receptor], LRPAP1 [low density lipoprotein receptor-related protein associated protein 1], LRPPRC [leucine-rich PPR-motif containing], LRRC37B [leucine rich repeat containing 37B], LRRC4C [leucine rich repeat containing 40], LRRTM1 [leucine rich repeat transmembrane neuronal 1], LSAMP [limbic system-associated membrane protein], LSM2 [LSM2 homolog, U6 small nuclear RNA associated (S. cerevisiae)], LSS [lanosterol synthase (2 [3-oxidosqualene-lanosterol cyclase)], LTA [lymphotoxin alpha (TNF superfamily, member 1)], LTA4H [leukotriene A4 hydrolase], LTBP1 [latent transforming growth factor beta binding protein 1], LTBP4 [latent transforming growth factor beta binding protein 4], LTBR [lymphotoxin beta receptor (TNFR superfamily, member 3)], LTC4S [leukotriene C4 synthase], LTF [lactotransferrin], LY96 [lymphocyte antigen 96], LYN [v-yes-1 Yamaguchi sarcoma viral related oncogene homolog], LYVE1 [lymphatic vessel endothelial hyaluronan receptor 1], M6PR [mannose-6-phosphate receptor (cation dependent)], MAB21L1 [mab-21-like 1 (C. elegans)], MAB21L2 [mab-2′-like 2 (C. elegans)], MAF [v-maf musculoaponeurotic fibrosarcoma oncogene homolog (avian)], MAG [myelin associated glycoprotein], MAGEA1 [melanoma antigen family A, 1 (directs expression of antigen MZ2-E)], MAGEL2 [MAGE-like 2], MAL [mal, T-cell differentiation protein], MAML2 [mastermind-like 2 (Drosophila)], MAN2A1 [mannosidase, alpha, class 2A, member 1], MANBA [mannosidase, beta A, lysosomal], MANF [mesencephalic astrocyte-derived neurotrophic factor], MAOA [monoamine oxidase A], MAOB [monoamine oxidase B], MAP1B [microtubule-associated protein 1B], MAP2 [microtubule-associated protein 2], MAP2K1 [mitogen-activated protein kinase kinase 1], MAP2K2 [mitogen-activated protein kinase kinase 2], MAP2K3 [mitogen-activated protein kinase kinase 3], MAP2K4 [mitogen-activated protein kinase kinase 4], MAP3K1 [mitogen-activated protein kinase kinase kinase 1], MAP3K12 [mitogen-activated protein kinase kinase kinase 12], MAP3K13 [mitogen-activated protein kinase kinase kinase 13], MAP3K14 [mitogen-activated protein kinase kinase kinase 14], MAP3K4 [mitogen-activated protein kinase kinase kinase 4], MAP3K7 [mitogen-activated protein kinase kinase kinase 7], MAPK1 [mitogen-activated protein kinase 1], MAPK10 [mitogen-activated protein kinase 10], MAPK14 [mitogen-activated protein kinase 14], MAPK3 [mitogen-activated protein kinase 3], MAPK8 [mitogen-activated protein kinase 8], MAPK81P2 [mitogen-activated protein kinase 8 interacting protein 2], MAPK81P3 [mitogen-activated protein kinase 8 interacting protein 3], MAPK9 [mitogen-activated protein kinase 9], MAPKAPK2 [mitogen-activated protein kinase-activated protein kinase 2], MAPKSP1 [MAPK scaffold protein 1], MAPRE3 [microtubule-associated protein, RP/EB family, member 3], MAPT [microtubule-associated protein tau], MARCKS [myristoylated alanine-rich protein kinase C substrate], MARK1 [MAP/microtubule affinity-regulating kinase 1], MARK2 [MAP/microtubule affinity-regulating kinase 2], MAT2A [methionine adenosyltransferase II, alpha], MATR3 [matrin 3], MAX [MYC associated factor X], MAZ [MYC-associated zinc finger protein (purine-binding transcription factor)], MB [myoglobin], MBD1 [methyl-CpG binding domain protein 1], MBD2 [methyl-CpG binding domain protein 2], MBD3 [methyl-CpG binding domain protein 3], MBD4 [methyl-CpG binding domain protein 4], MBL2 [mannose-binding lectin (protein C) 2, soluble (opsonic defect)], MBP [myelin basic protein], MBTPS1 [membrane-bound transcription factor peptidase, site 1], MC1R [melanocortin 1 receptor (alpha melanocyte stimulating hormone receptor)], MC3R [melanocortin 3 receptor], MC4R [melanocortin 4 receptor], MCCC2 [methylcrotonoyl-Coenzyme A carboxylase 2 (beta)], MCF2L [MCF.2 cell line derived transforming sequence-like], MCHR1 [melanin-concentrating hormone receptor 1], MCL1 [myeloid cell leukemia sequence 1 (BCL2-related)], MCM7 [minichromosome maintenance complex component 7], MCPH1 [microcephalin 1], MDC1 [mediator of DNA-damage checkpoint 1], MDFIC [MyoD family inhibitor domain containing], MDGA1 [MAM domain containing glycosylphosphatidylinositol anchor 1], MDK [midkine (neurite growth-promoting factor 2)], MDM2 [Mdm2 p53 binding protein homolog (mouse)], ME2 [malic enzyme 2, NAD(+)-dependent, mitochondrial], MECP2 [methyl CpG binding protein 2 (Rett syndrome)], MED1 [mediator complex subunit 1], MED12 [mediator complex subunit 12], MED24 [mediator complex subunit 24], MEF2A [myocyte enhancer factor 2A], MEF2C [myocyte enhancer factor 20], MEIS1 [Meis homeobox 1], MEN1 [multiple endocrine neoplasia I], MERTK [c-mer proto-oncogene tyrosine kinase], MESP2 [mesoderm posterior 2 homolog (mouse)], MEST [mesoderm specific transcript homolog (mouse)], MET [met proto-oncogene (hepatocyte growth factor receptor)], METAP2 [methionyl aminopeptidase 2], METRN [meteorin, glial cell differentiation regulator], MFSD6 [major facilitator superfamily domain containing 6], MGAT2 [mannosyl (alpha-1 [6-)-glycoprotein beta-1 [2-N-acetylglucosaminyltransferase], MGMT [0-6-methylguanine-DNA methyltransferase], MGP [matrix Gla protein], MGST1 [microsomal glutathione S-transferase 1], MICA [MHC class I polypeptide-related sequence A], MICAL1 [microtubule associated monoxygenase, calponin and LIM domain containing 1], MICB [MHC class I polypeptide-related sequence B], MIF [macrophage migration inhibitory factor (glycosylation-inhibiting factor)], MITF [microphthalmia-associated transcription factor], MKI67 [antigen identified by monoclonal antibody Ki-67], MKKS [McKusick-Kaufman syndrome], MKNK1 [MAP kinase interacting serine/threonine kinase 1], MKRN3 [makorin ring finger protein 3], MKS1 [Meckel syndrome, type 1], MLH1 [mutL homolog 1, colon cancer, nonpolyposis type 2 (E. coli)], MLL [myeloid/lymphoid or mixed-lineage leukemia (trithorax homolog, Drosophila)], MLLT4 [myeloid/lymphoid or mixed-lineage leukemia (trithorax homolog, Drosophila); translocated to, 4], MLPH [melanophilin], MLX [MAX-like protein X], MLXIPL [MLX interacting protein-like], MME [membrane metallo-endopeptidase], MMP1 [matrix metallopeptidase 1 (interstitial collagenase)], MMP10 [matrix metallopeptidase 10 (stromelysin 2)], MMP12 [matrix metallopeptidase 12 (macrophage elastase)], MMP13 [matrix metallopeptidase 13 (collagenase 3)], MMP14 [matrix metallopeptidase 14 (membrane-inserted)], MMP2 [matrix metallopeptidase 2 (gelatinase A, 72 kDa gelatinase, 72 kDa type IV collagenase)], MMP24 [matrix metallopeptidase 24 (membrane-inserted)], MMP26 [matrix metallopeptidase 26], MMP3 [matrix metallopeptidase 3 (stromelysin 1, progelatinase)], MMP7 [matrix metallopeptidase 7 (matrilysin, uterine)], MMP8 [matrix metallopeptidase 8 (neutrophil collagenase)], MMP9 [matrix metallopeptidase 9 (gelatinase B, 92 kDa gelatinase, 92 kDa type IV collagenase)], MN1 [meningioma (disrupted in balanced translocation) 1], MNAT1 [menage a trois homolog 1, cyclin H assembly factor (Xenopus laevis)], MNX1 [motor neuron and pancreas homeobox 1], MOG [myelin oligodendrocyte glycoprotein], MPL [myeloproliferative leukemia virus oncogene], MPO [myeloperoxidase], MPP1 [membrane protein, palmitoylated 1, 55 kDa], MPZL1 [myelin protein zero-like 1], MR1 [major histocompatibility complex, class I-related], MRAP [melanocortin 2 receptor accessory protein], MRAS [muscle RAS oncogene homolog], MRC1 [mannose receptor, C type 1], MRGPRX1 [MAS-related GPR, member X1], MS4A1 [membrane-spanning 4-domains, subfamily A, member 1], MSH2 [mutS homolog 2, colon cancer, nonpolyposis type 1 (E. coli)], MSH3 [mutS homolog 3 (E. coli)], MSI1 [musashi homolog 1 (Drosophila)], MSN [moesin], MSR1 [macrophage scavenger receptor 1], MSTN [myostatin], MSX1 [msh homeobox 1], MSX2 [msh homeobox 2], MT2A [metallothionein 2A], MT3 [metallothionein 3], MT-ATP6 [mitochondrially encoded ATP synthase 6], MT-001 [mitochondrially encoded cytochrome c oxidase I], MT-CO2 [mitochondrially encoded cytochrome c oxidase II], MT-CO3 [mitochondrially encoded cytochrome c oxidase III], MTF1 [metal-regulatory transcription factor 1], MTHFD1 [methylenetetrahydrofolate dehydrogenase (NADP+ dependent) 1, methenyltetrahydrofolate cyclohydrolase, formyltetrahydrofolate synthetase], MTHFD1L [methylenetetrahydrofolate dehydrogenase (NADP+ dependent) 1-like], MTHFR [5 [10-methylenetetrahydrofolate reductase (NADPH)], MTL5 [metallothionein-like 5, testis-specific (tesmin)], MTMR14 [myotubularin related protein 14], MT-ND6 [mitochondrially encoded NADH dehydrogenase 6], MTNR1A [melatonin receptor 1A], MTNR1B [melatonin receptor 1B], MTOR [mechanistic target of rapamycin (serine/threonine kinase)], MTR [5-methyltetrahydrofolate-homocysteine methyltransferase], MTRR [5-methyltetrahydrofolate-homocysteine methyltransferase reductase], MTTP [microsomal triglyceride transfer protein], MUC1 [mucin 1, cell surface associated], MUC16 [mucin 16, cell surface associated], MUC19 [mucin 19, oligomeric], MUC2 [mucin 2, oligomeric mucus/gel-forming], MUC3A [mucin 3A, cell surface associated], MUC5AC [mucin 5AC, oligomeric mucus/gel-forming], MUSK [muscle, skeletal, receptor tyrosine kinase], MUT [methylmalonyl Coenzyme A mutase], MVK [mevalonate kinase], MVP [major vault protein], MX1 [myxovirus (influenza virus) resistance 1, interferon-inducible protein p78 (mouse)], MXD1 [MAX dimerization protein 1], MXI1 [MAX interactor 1], MYB [v-myb myeloblastosis viral oncogene homolog (avian)], MYC [v-myc myelocytomatosis viral oncogene homolog (avian)], MYCBP2 [MYC binding protein 2], MYCN [v-myc myelocytomatosis viral related oncogene, neuroblastoma derived (avian)], MYD88 [myeloid differentiation primary response gene (88)], MYF5 [myogenic factor 5], MYH10 [myosin, heavy chain 10, non-muscle], MYH14 [myosin, heavy chain 14, non-muscle], MYH7 [myosin, heavy chain 7, cardiac muscle, beta], MYL1 [myosin, light chain 1, alkali; skeletal, fast], MYL10 [myosin, light chain 10, regulatory], MYL12A [myosin, light chain 12A, regulatory, non-sarcomeric], MYL12B [myosin, light chain 12B, regulatory], MYL2 [myosin, light chain 2, regulatory, cardiac, slow], MYL3 [myosin, light chain 3, alkali; ventricular, skeletal, slow], MYL4 [myosin, light chain 4, alkali; atrial, embryonic], MYL5 [myosin, light chain 5, regulatory], MYL6 [myosin, light chain 6, alkali, smooth muscle and non-muscle], MYL6B [myosin, light chain 6B, alkali, smooth muscle and non-muscle], MYL7 [myosin, light chain 7, regulatory], MYL9 [myosin, light chain 9, regulatory], MYLK [myosin light chain kinase], MYLPF [myosin light chain, phosphorylatable, fast skeletal muscle], MYO1D [myosin ID], MYO5A [myosin VA (heavy chain 12, myoxin)], MYOC [myocilin, trabecular meshwork inducible glucocorticoid response], MYOD1 [myogenic differentiation 1], MYOG [myogenin (myogenic factor 4)], MYOM2 [myomesin (M-protein) 2, 165 kDa], MYST3 [MYST histone acetyltransferase (monocytic leukemia) 3], NACA [nascent polypeptide-associated complex alpha subunit], NAGLU [N-acetylglucosaminidase, alpha-], NAIP [NLR family, apoptosis inhibitory protein], NAMPT [nicotinamide phosphoribosyltransferase], NANOG [Nanog homeobox], NANS [N-acetylneuraminic acid synthase], NAP1L2 [nucleosome assembly protein 1-like 2], NAPA [N-ethylmaleimide-sensitive factor attachment protein, alpha], NAPG [N-ethylmaleimide-sensitive factor attachment protein, gamma], NAT2 [N-acetyltransferase 2 (arylamine N-acetyltransferase)], NAV1 [neuron navigator 1], NAV3 [neuron navigator 3], NBEA [neurobeachin], NCALD [neurocalcin delta], NCAM1 [neural cell adhesion molecule 1], NCAM2 [neural cell adhesion molecule 2], NCF1 [neutrophil cytosolic factor 1], NCF2 [neutrophil cytosolic factor 2], NCK1 [NCK adaptor protein 1], NCK2 [NCK adaptor protein 2 ], NCKAP1 [NCK-associated protein 1], NCL [nucleolin], NCOA2 [nuclear receptor coactivator 2], NCOA3 [nuclear receptor coactivator 3], NCOR1 [nuclear receptor co-repressor 1], NCOR2 [nuclear receptor co-repressor 2], NDE1 [nudE nuclear distribution gene E homolog 1 (A. nidulans)], NDEL1 [nudE nuclear distribution gene E homolog (A. nidulans)-like 1], NDN [necdin homolog (mouse)], NDNL2 [necdin-like 2], NDP [Norrie disease (pseudoglioma)], NDUFA1 [NADH dehydrogenase (ubiquinone) 1 alpha subcomplex, 1, 7.5 kDa], NDUFAB1 [NADH dehydrogenase (ubiquinone) 1, alpha/beta subcomplex, 1, 8 kDa], NDUFS3 [NADH dehydrogenase (ubiquinone) Fe-S protein 3, 30 kDa (NADH-coenzyme Q reductase)], NDUFV3 [NADH dehydrogenase (ubiquinone) flavoprotein 3, 10 kDa], NEDD4 [neural precursor cell expressed, developmentally down-regulated 4], NEDD4L [neural precursor cell expressed, developmentally down-regulated 4-like], NEFH [neurofilament, heavy polypeptide], NEFL [neurofilament, light polypeptide], NEFM [neurofilament, medium polypeptide], NENF [neuron derived neurotrophic factor], NEO1 [neogenin homolog 1 (chicken)], NES [nestin], NET1 [neuroepithelial cell transforming 1], NEU1 [sialidase 1 (lysosomal sialidase)], NEU3 [sialidase 3 (membrane sialidase)], NEUROD1 [neurogenic differentiation 1], NEUROD4 [neurogenic differentiation 4], NEUROG1 [neurogenin 1], NEUROG2 [neurogenin 2], NF1 [neurofibromin 1], NF2 [neurofibromin 2 (merlin)], NFASC [neurofascin homolog (chicken)], NFAT5 [nuclear factor of activated T-cells 5, tonicity-responsive], NFATC1 [nuclear factor of activated T-cells, cytoplasmic, calcineurin-dependent 1], NFATC2 [nuclear factor of activated T-cells, cytoplasmic, calcineurin-dependent 2], NFATC3 [nuclear factor of activated T-cells, cytoplasmic, calcineurin-dependent 3], NFATC4 [nuclear factor of activated T-cells, cytoplasmic, calcineurin-dependent 4], NFE2L2 [nuclear factor (erythroid-derived 2)-like 2], NFIC [nuclear factor I/C (CCAAT-binding transcription factor)], NFIL3 [nuclear factor, interleukin 3 regulated], NFKB1 [nuclear factor of kappa light polypeptide gene enhancer in B-cells 1], NFKB2 [nuclear factor of kappa light polypeptide gene enhancer in B-cells 2 (p49/p100)], NFKBIA [nuclear factor of kappa light polypeptide gene enhancer in B-cells inhibitor, alpha], NFKBIB [nuclear factor of kappa light polypeptide gene enhancer in B-cells inhibitor, beta], NFKBIL1 [nuclear factor of kappa light polypeptide gene enhancer in B-cells inhibitor-like 1], NFYA [nuclear transcription factor Y, alpha], NFYB [nuclear transcription factor Y, beta], NGEF [neuronal guanine nucleotide exchange factor], NGF [nerve growth factor (beta polypeptide)], NGFR [nerve growth factor receptor (TNFR superfamily, member 16)], NGFRAP1 [nerve growth factor receptor (TNFRSF16) associated protein 1], NHLRC1 [NHL repeat containing 1], NINJ1 [ninjurin 1], NINJ2 [ninjurin 2], NIP7 [nuclear import 7 homolog (S. cerevisiae)], NIPA1 [non imprinted in Prader-Willi/Angelman syndrome 1], NIPA2 [non imprinted in Prader-Willi/Angelman syndrome 2], NIPAL1 [NIPA-like domain containing 1], NIPAL4 [NIPA-like domain containing 4], NIPSNAP1 [nipsnap homolog 1 (C. elegans)], NISCH [nischarin], NIT2 [nitrilase family, member 2], NKX2-1 [NK2 homeobox 1], NKX2-2 [NK2 homeobox 2], NLGN1 [neuroligin 1], NLGN2 [neuroligin 2], NLGN3 [neuroligin 3], NLGN4X [neuroligin 4, X-linked], NLGN4Y [neuroligin 4, Y-linked], NLRP3 [NLR family, pyrin domain containing 3], NMB [neuromedin B], NME1 [non-metastatic cells 1, protein (NM23A) expressed in], NME2 [non-metastatic cells 2, protein (NM23B) expressed in], NME4 [non-metastatic cells 4, protein expressed in], NNAT [neuronatin], NOD1 [nucleotide-binding oligomerization domain containing 1], NOD2 [nucleotide-binding oligomerization domain containing 2], NOG [noggin], NOL6 [nucleolar protein family 6 (RNA-associated)], NOS1 [nitric oxide synthase 1 (neuronal)], NOS2 [nitric oxide synthase 2, inducible], NOS3 [nitric oxide synthase 3 (endothelial cell)], NOSTRIN [nitric oxide synthase trafficker], NOTCH1 [Notch homolog 1, translocation-associated (Drosophila)], NOTCH2 [Notch homolog 2 (Drosophila)], NOTCH3 [Notch homolog 3 (Drosophila)], NOV [nephroblastoma overexpressed gene], NOVA1 [neuro-oncological ventral antigen 1], NOVA2 [neuro-oncological ventral antigen 2], NOX4 [NADPH oxidase 4], NPAS4 [neuronal PAS domain protein 4], NPFF [neuropeptide FF-amide peptide precursor], NPHP1 [nephronophthisis 1 (juvenile)], NPHP4 [nephronophthisis 4], NPHS1 [nephrosis 1, congenital, Finnish type (nephrin)], NPM1 [nucleophosmin (nucleolar phosphoprotein B23, numatrin)], NPPA [natriuretic peptide precursor A], NPPB [natriuretic peptide precursor B], NPPC [natriuretic peptide precursor C], NPR1 [natriuretic peptide receptor A/guanylate cyclase A (atrionatriuretic peptide receptor A)], NPR3 [natriuretic peptide receptor C/guanylate cyclase C (atrionatriuretic peptide receptor C)], NPRL2 [nitrogen permease regulator-like 2 (S. cerevisiae)], NPTX1 [neuronal pentraxin I], NPTX2 [neuronal pentraxin II], NPY [neuropeptide Y], NPY1R [neuropeptide Y receptor Y1], NPY2R [neuropeptide Y receptor Y2], NPY5R [neuropeptide Y receptor Y5], NQO1 [NAD(P)H dehydrogenase, quinone 1], NQO2 [NAD(P)H dehydrogenase, quinone 2], NR0B1 [nuclear receptor subfamily 0, group B, member 1], NR0B2 [nuclear receptor subfamily 0, group B, member 2], NR1H3 [nuclear receptor subfamily 1, group H, member 3], NR1H4 [nuclear receptor subfamily 1, group H, member 4], NR1I2 [nuclear receptor subfamily 1, group I, member 2], NR1I3 [nuclear receptor subfamily 1, group I, member 3], NR2C1 [nuclear receptor subfamily 2, group C, member 1], NR2C2 [nuclear receptor subfamily 2, group C, member 2], NR2E1 [nuclear receptor subfamily 2, group E, member 1], NR2F1 [nuclear receptor subfamily 2, group F, member 1], NR2F2 [nuclear receptor subfamily 2, group F, member 2], NR3C1 [nuclear receptor subfamily 3, group C, member 1 (glucocorticoid receptor)], NR3C2 [nuclear receptor subfamily 3, group C, member 2], NR4A2 [nuclear receptor subfamily 4, group A, member 2], NR4A3 [nuclear receptor subfamily 4, group A, member 3], NR5A1 [nuclear receptor subfamily 5, group A, member 1], NR6A1 [nuclear receptor subfamily 6, group A, member 1], NRAS [neuroblastoma RAS viral (v-ras) oncogene homolog], NRCAM [neuronal cell adhesion molecule], NRD1 [nardilysin (N-arginine dibasic convertase)], NRF1 [nuclear respiratory factor 1], NRG1 [neuregulin 1], NRIP1 [nuclear receptor interacting protein 1], NRN1 [neuritin 1], NRP1 [neuropilin 1], NRP2 [neuropilin 2], NRSN1 [neurensin 1], NRTN [neurturin], NRXN1 [neurexin 1], NRXN3 [neurexin 3], NSD1 [nuclear receptor binding SET domain protein 1], NSF [N-ethylmaleimide-sensitive factor], NSUN5 [NOP2/Sun domain family, member 5], NT5E [5′-nucleotidase, ecto (CD73)], NTF3 [neurotrophin 3], NTF4 [neurotrophin 4], NTHL1 [nth endonuclease III-like 1 (E. coli)], NTN1 [netrin 1], NTN3 [netrin 3], NTN4 [netrin 4], NTNG1 [netrin G1], NTRK1 [neurotrophic tyrosine kinase, receptor, type 1], NTRK2 [neurotrophic tyrosine kinase, receptor, type 2], NTRK3 [neurotrophic tyrosine kinase, receptor, type 3], NTS [neurotensin], NTSR1 [neurotensin receptor 1 (high affinity)], NUCB2 [nucleobindin 2], NUDC [nuclear distribution gene C homolog (A. nidulans)], NUDT6 [nudix (nucleoside diphosphate linked moiety X)-type motif 6], NUDT7 [nudix (nucleoside diphosphate linked moiety X)-type motif 7], NUMB [numb homolog (Drosophila)], NUP98 [nucleoporin 98 kDa], NUPR1 [nuclear protein, transcriptional regulator, 1], NXF1 [nuclear RNA export factor 1], NXNL1 [nucleoredoxin-like 1], OAT [ornithine aminotransferase], OCA2 [oculocutaneous albinism II], OCLN [occludin], OCM [oncomodulin], ODC1 [ornithine decarboxylase 1], OFD1 [oral-facial-digital syndrome 1], OGDH [oxoglutarate (alpha-ketoglutarate) dehydrogenase (lipoamide)], OLA1 [Obg-like ATPase 1], OLIG1 [oligodendrocyte transcription factor 1], OLIG2 [oligodendrocyte lineage transcription factor 2], OLR1 [oxidized low density lipoprotein (lectin-like) receptor 1], OMG [oligodendrocyte myelin glycoprotein], OPHN1 [oligophrenin 1], OPN1SW [opsin 1 (cone pigments), short-wave-sensitive], OPRD1 [opioid receptor, delta 1], OPRK1 [opioid receptor, kappa 1], OPRL1 [opiate receptor-like 1], OPRM1 [opioid receptor, mu 1], OPTN [optineurin], OSBP [oxysterol binding protein], OSBPL10 [oxysterol binding protein-like 10], OSBPL6 [oxysterol binding protein-like 6], OSM [oncostatin M], OTC [ornithine carbamoyltransferase], OTX2 [orthodenticle homeobox 2], OXA1L [oxidase (cytochrome c) assembly 1-like], OXT [oxytocin, prepropeptide], OXTR [oxytocin receptor], P2RX7 [purinergic receptor P2X, ligand-gated ion channel, 7], P2RY1 [purinergic receptor P2Y, G-protein coupled, 1], P2RY12 [purinergic receptor P2Y, G-protein coupled, 12], P2RY2 [purinergic receptor P2Y, G-protein coupled, 2], P4HB [prolyl 4-hydroxylase, beta polypeptide], PABPC1 [poly(A) binding protein, cytoplasmic 1], PADI4 [peptidyl arginine deiminase, type IV], PAEP [progestagen-associated endometrial protein], PAFAH1B1 [platelet-activating factor acetylhydrolase 1b, regulatory subunit 1 (45 kDa)], PAFAH1B2 [platelet-activating factor acetylhydrolase 1b, catalytic subunit 2 (30 kDa)], PAG1 [phosphoprotein associated with glycosphingolipid microdomains 1], PAH [phenylalanine hydroxylase], PAK1 [p21 protein (Cdc42/Rac)-activated kinase 1], PAK2 [p21 protein (Cdc42/Rac)-activated kinase 2], PAK3 [p21 protein (Cdc42/Rac)-activated kinase 3], PAK-4 [p21 protein (Cdc42/Rac)-activated kinase 4], PAK6 [p21 protein (Cdc42/Rac)-activated kinase 6], PAK7 [p21 protein (Cdc42/Rac)-activated kinase 7], PAPPA [pregnancy-associated plasma protein A, pappalysin 1], PAPPA2 [pappalysin 2], PARD6A [par-6 partitioning defective 6 homolog alpha (C. elegans)], PARG [poly (ADP-ribose) glycohydrolase], PARK2 [Parkinson disease (autosomal recessive, juvenile) 2, parkin], PARK7 [Parkinson disease (autosomal recessive, early onset) 7], PARN [poly(A)-specific ribonuclease (deadenylation nuclease)], PARP1 [poly (ADP-ribose) polymerase 1], PAWR [PRKC, apoptosis, WT1, regulator], PAX2 [paired box 2], PAX3 [paired box 3], PAX5 [paired box 5], PAX6 [paired box 6], PAX7 [paired box 7], PBX1 [pre-B-cell leukemia homeobox 1], PC [pyruvate carboxylase], PCDH10 [protocadherin 10], PCDH19 [protocadherin 19], PCDHA12 [protocadherin alpha 12], PCK2 [phosphoenolpyruvate carboxykinase 2 (mitochondrial)], POLO [piccolo (presynaptic cytomatrix protein)], PCM1 [pericentriolar material 1], PCMT1 [protein-L-isoaspartate (D-aspartate) O-methyltransferase], PCNA [proliferating cell nuclear antigen], PCNT [pericentrin], PCP4 [Purkinje cell protein 4], PCSK7 [proprotein convertase subtilisin/kexin type 7], PDCD1 [programmed cell death 1], PDE11A [phosphodiesterase 11A], PDE3B [phosphodiesterase 3B, cGMP-inhibited], PDE4A [phosphodiesterase 4A, cAMP-specific (phosphodiesterase E2 dunce homolog, Drosophila)], PDE4B [phosphodiesterase 4B, cAMP-specific (phosphodiesterase E4 dunce homolog, Drosophila)], PDE4D [phosphodiesterase 4D, cAMP-specific (phosphodiesterase E3 dunce homolog, Drosophila)], PDE5A [phosphodiesterase 5A, cGMP-specific], PDE8A [phosphodiesterase 8A], PDGFA [platelet-derived growth factor alpha polypeptide], PDGFB [platelet-derived growth factor beta polypeptide (simian sarcoma viral (v-sis) oncogene homolog)], PDGFC [platelet derived growth factor C], PDGFD [platelet derived growth factor D], PDGFRA [platelet-derived growth factor receptor, alpha polypeptide], PDGFRB [platelet-derived growth factor receptor, beta polypeptide], PDHA1 [pyruvate dehydrogenase (lipoamide) alpha 1], PDIA2 [protein disulfide isomerase family A, member 2], PDIA3 [protein disulfide isomerase family A, member 3], PDLIM1 [PDZ and LIM domain 1], PDLIM7 [PDZ and LIM domain 7 (enigma)], PDP1 [pyruvate dehyrogenase phosphatase catalytic subunit 1], PDPN [podoplanin], PDXK [pyridoxal (pyridoxine, vitamin B6) kinase], PDXP [pyridoxal (pyridoxine, vitamin B6) phosphatase], PDYN [prodynorphin], PDZK1 [PDZ domain containing 1], PEBP1 [phosphatidylethanolamine binding protein 1], PECAM1 [platelet/endothelial cell adhesion molecule], PENK [proenkephalin], PER1 [period homolog 1 (Drosophila)], PER2 [period homolog 2 (Drosophila)], PEX13 [peroxisomal biogenesis factor 13], PEX2 [peroxisomal biogenesis factor 2], PEX5 [peroxisomal biogenesis factor 5], PEX7 [peroxisomal biogenesis factor 7], PF4 [platelet factor 4], PFAS [phosphoribosylformylglycinamidine synthase], PFKL [phosphofructokinase, liver], PFKM [phosphofructokinase, muscle], PFN1 [profilin 1], PFN2 [profilin 2], PFN3 [profilin 3], PFN4 [profilin family, member 4], PGAM2 [phosphoglycerate mutase 2 (muscle)], PGD [phosphogluconate dehydrogenase], PGF [placental growth factor], PGK1 [phosphoglycerate kinase 1], PGM1 [phosphoglucomutase 1], PGR [progesterone receptor], PHB [prohibitin], PHEX [phosphate regulating endopeptidase homolog, X-linked], PHF10 [PHD finger protein 10], PHF8 [PHD finger protein 8], PHGDH [phosphoglycerate dehydrogenase], PHKA2 [phosphorylase kinase, alpha 2 (liver)], PHLDA2 [pleckstrin homology-like domain, family A, member 2], PHOX2B [paired-like homeobox 2b], PHYH [phytanoyl-CoA 2-hydroxylase], PHYHIP [phytanoyl-CoA 2-hydroxylase interacting protein], PIAS1 [protein inhibitor of activated STAT, 1], PICALM [phosphatidylinositol binding clathrin assembly protein], P1GF [phosphatidylinositol glycan anchor biosynthesis, class F], PIGP [phosphatidylinositol glycan anchor biosynthesis, class P], PIK3C2A [phosphoinositide-3-kinase, class 2, alpha polypeptide], PIK3C2B [phosphoinositide-3-kinase, class 2, beta polypeptide], PIK3C2G [phosphoinositide-3-kinase, class 2, gamma polypeptide], PIK3C3 [phosphoinositide-3-kinase, class 3], PIK3CA [phosphoinositide-3-kinase, catalytic, alpha polypeptide], PIK3CB [phosphoinositide-3-kinase, catalytic, beta polypeptide], PIK3CD [phosphoinositide-3-kinase, catalytic, delta polypeptide], PIK3CG [phosphoinositide-3-kinase, catalytic, gamma polypeptide], PIK3R1 [phosphoinositide-3-kinase, regulatory subunit 1 (alpha)], PIK3R2 [phosphoinositide-3-kinase, regulatory subunit 2 (beta)], PIK3R3 [phosphoinositide-3-kinase, regulatory subunit 3 (gamma)], PIK3R4 [phosphoinositide-3-kinase, regulatory subunit 4], PIK3R5 [phosphoinositide-3-kinase, regulatory subunit 5], PINK1 [PTEN induced putative kinase 1], PITX1 [paired-like homeodomain 1], PITX2 [paired-like homeodomain 2], PITX3 [paired-like homeodomain 3], PKD1 [polycystic kidney disease 1 (autosomal dominant)], PKD2 [polycystic kidney disease 2 (autosomal dominant)], PKHD1 [polycystic kidney and hepatic disease 1 (autosomal recessive)], PKLR [pyruvate kinase, liver and RBC], PKN2 [protein kinase N2], PKNOX1 [PBX/knotted 1 homeobox 1], PL-5283 [PL-5283 protein], PLA2G10 [phospholipase A2, group X], PLA2G2A [phospholipase A2, group IIA (platelets, synovial fluid)], PLA2G4A [phospholipase A2, group IVA (cytosolic, calcium-dependent)], PLA2G6 [phospholipase A2, group VI (cytosolic, calcium-independent)], PLA2G7 [phospholipase A2, group VII (platelet-activating factor acetylhydrolase, plasma)], PLAC4 [placenta-specific 4], PLAG1 [pleiomorphic adenoma gene 1], PLAGL1 [pleiomorphic adenoma gene-like 1], PLAT [plasminogen activator, tissue], PLAU [plasminogen activator, urokinase], PLAUR [plasminogen activator, urokinase receptor], PLCB1 [phospholipase C, beta 1 (phosphoinositide-specific)], PLCB2 [phospholipase C, beta 2], PLCB3 [phospholipase C, beta 3 (phosphatidylinositol-specific)], PLCB4 [phospholipase C, beta 4], PLCG1 [phospholipase C, gamma 1], PLCG2 [phospholipase C, gamma 2 (phosphatidylinositol-specific)], PLCL1 [phospholipase C-like 1], PLD1 [phospholipase D1, phosphatidylcholine-specific], PLD2 [phospholipase D2], PLEK [pleckstrin], PLEKHH1 [pleckstrin homology domain containing, family H (with MyTH4 domain) member 1], PLG [plasminogen], PLIN1 [perilipin 1], PLK1 [polo-like kinase 1 (Drosophila)], PLOD1 [procollagen-lysine 1,2-oxoglutarate 5-dioxygenase 1], PLP1 [proteolipid protein 1], PLTP [phospholipid transfer protein], PLXNA1 [plexin A1], PLXNA2 [plexin A2], PLXNA3 [plexin A3], PLXNA4 [plexin A4], PLXNB1 [plexin B1], PLXNB2 [plexin B2], PLXNB3 [plexin B3], PLXNC1 [plexin C1], PLXND1 [plexin D1], PML [promyelocytic leukemia], PMP2 [peripheral myelin protein 2], PMP22 [peripheral myelin protein 22], PMS2 [PMS2 postmeiotic segregation increased 2 (S. cerevisiae)], PMVK [phosphomevalonate kinase], PNOC [prepronociceptin], PNP [purine nucleoside phosphorylase], PNPLA6 [patatin-like phospholipase domain containing 6], PNPO [pyridoxamine 5′-phosphate oxidase], POFUT2 [protein O-fucosyltransferase 2], POLB [polymerase (DNA directed), beta], POLR1C [polymerase (RNA) I polypeptide C, 30 kDa], POLR2A [polymerase (RNA) II (DNA directed) polypeptide A, 220 kDa], POLR3K [polymerase (RNA) III (DNA directed) polypeptide K, 12.3 kDa], POM121C [POM121 membrane glycoprotein C], POMC [proopiomelanocortin], POMGNT1 [protein O-linked mannose beta1 [2-N-acetylglucosaminyltransferase], POMT1 [protein-O-mannosyltransferase 1], PON1 [paraoxonase 1], PON2 [paraoxonase 2], POR [P450 (cytochrome) oxidoreductase], POSTN [periostin, osteoblast specific factor], POU1F1 [POU class 1 homeobox 1], POU2F1 [POU class 2 homeobox 1], POU3F4 [POU class 3 homeobox 4], POU4F1 [POU class 4 homeobox 1], POU4F2 [POU class 4 homeobox 2], POU4F3 [POU class 4 homeobox 3], POU5F1 [POU class 5 homeobox 1], PPA1 [pyrophosphatase (inorganic) 1], PPARA [peroxisome proliferator-activated receptor alpha], PPARD [peroxisome proliferator-activated receptor delta], PPARG [peroxisome proliferator-activated receptor gamma], PPARGC1A [peroxisome proliferator-activated receptor gamma, coactivator 1 alpha], PPAT [phosphoribosyl pyrophosphate amidotransferase], PPBP [pro-platelet basic protein (chemokine (C-X-C motif) ligand 7)], PPFIA1 [protein tyrosine phosphatase, receptor type, f polypeptide (PTPRF), interacting protein (liprin), alpha 1], PPFIA2 [protein tyrosine phosphatase, receptor type, f polypeptide (PTPRF), interacting protein (liprin), alpha 2], PPFIA3 [protein tyrosine phosphatase, receptor type, f polypeptide (PTPRF), interacting protein (liprin), alpha 3], PPFIBP1 [PTPRF interacting protein, binding protein 1 (liprin beta 1)], PPIC [peptidylprolyl isomerase C (cyclophilin C)], PPIG [peptidylprolyl isomerase G (cyclophilin G)], PPP1R15A [protein phosphatase 1, regulatory (inhibitor) subunit 15A], PPP1R1B [protein phosphatase 1, regulatory (inhibitor) subunit 1B], PPP1R9A [protein phosphatase 1, regulatory (inhibitor) subunit 9A], PPP1R9B [protein phosphatase 1, regulatory (inhibitor) subunit 9B], PPP2CA [protein phosphatase 2, catalytic subunit, alpha isozyme], PPP2R4 [protein phosphatase 2A activator, regulatory subunit 4], PPP3CA [protein phosphatase 3, catalytic subunit, alpha isozyme], PPP3CB [protein phosphatase 3, catalytic subunit, beta isozyme], PPP3CC [protein phosphatase 3, catalytic subunit, gamma isozyme], PPP3R1 [protein phosphatase 3, regulatory subunit B, alpha], PPP3R2 [protein phosphatase 3, regulatory subunit B, beta], PPP4C [protein phosphatase 4, catalytic subunit], PPY [pancreatic polypeptide], PQBP1 [polyglutamine binding protein 1], PRAM1 [PML-RARA regulated adaptor molecule 1], PRAME [preferentially expressed antigen in melanoma], PRDM1 [PR domain containing 1, with ZNF domain], PRDM15 [PR domain containing 15], PRDM2 [PR domain containing 2, with ZNF domain], PRDX1 [peroxiredoxin 1], PRDX2 [peroxiredoxin 2], PRDX3 [peroxiredoxin 3], PRDX4 [peroxiredoxin 4], PRDX6 [peroxiredoxin 6], PRF1 [perforin 1 (pore forming protein)], PRKAA1 [protein kinase, AMP-activated, alpha 1 catalytic subunit], PRKAA2 [protein kinase, AMP-activated, alpha 2 catalytic subunit], PRKAB1 [protein kinase, AMP-activated, beta 1 non-catalytic subunit], PRKACA [protein kinase, cAMP-dependent, catalytic, alpha], PRKACB [protein kinase, cAMP-dependent, catalytic, beta], PRKACG [protein kinase, cAMP-dependent, catalytic, gamma], PRKAG1 [protein kinase, AMP-activated, gamma 1 non-catalytic subunit], PRKAG2 [protein kinase, AMP-activated, gamma 2 non-catalytic subunit], PRKAR1A [protein kinase, cAMP-dependent, regulatory, type I, alpha (tissue specific extinguisher 1)], PRKAR1B [protein kinase, cAMP-dependent, regulatory, type I, beta], PRKAR2A [protein kinase, cAMP-dependent, regulatory, type II, alpha], PRKAR2B [protein kinase, cAMP-dependent, regulatory, type II, beta], PRKCA [protein kinase C, alpha], PRKCB [protein kinase C, beta], PRKCD [protein kinase C, delta], PRKCE [protein kinase C, epsilon], PRKCG [protein kinase C, gamma], PRKCH [protein kinase C, eta], PRKCI [protein kinase C, iota], PRKCQ [protein kinase C, theta], PRKCZ [protein kinase C, zeta], PRKD1 [protein kinase D1], PRKDC [protein kinase, DNA-activated, catalytic polypeptide], PRKG1 [protein kinase, cGMP-dependent, type I], PRL [prolactin], PRLR [prolactin receptor], PRMT1 [protein arginine methyltransferase 1], PRNP [prion protein], PROC [protein C (inactivator of coagulation factors Va and VIIIa)], PROCR [protein C receptor, endothelial (EPCR)], PRODH [proline dehydrogenase (oxidase) 1], PROK1 [prokineticin 1], PROK2 [prokineticin 2], PROM1 [prominin 1], PRO51 [protein S (alpha)], PRPF40A [PRP40 pre-mRNA processing factor 40 homolog A (S. cerevisiae)], PRPF40B [PRP40 pre-mRNA processing factor 40 homolog B (S. cerevisiae)], PRPH [peripherin], PRPH2 [peripherin 2 (retinal degeneration, slow)], PRPS1 [phosphoribosyl pyrophosphate synthetase 1], PRRG4 [proline rich Gla (G-carboxyglutamic acid) 4 (transmembrane)], PRSS8 [protease, serine, 8], PRTN3 [proteinase 3], PRX [periaxin], PSAP [prosaposin], PSEN1 [presenilin 1], PSEN2 [presenilin 2 (Alzheimer disease 4)], PSG1 [pregnancy specific beta-1-glycoprotein 1], PSIP1 [PC4 and SFRS1 interacting protein 1], PSMA5 [proteasome (prosome, macropain) subunit, alpha type, 5], PSMA6 [proteasome (prosome, macropain) subunit, alpha type, 6], PSMB8 [proteasome (prosome, macropain) subunit, beta type, 8 (large multifunctional peptidase 7)], PSMB9 [proteasome (prosome, macropain) subunit, beta type, 9 (large multifunctional peptidase 2)], PSMC1 [proteasome (prosome, macropain) 26S subunit, ATPase, 1], PSMC4 [proteasome (prosome, macropain) 26S subunit, ATPase, 4], PSMD9 [proteasome (prosome, macropain) 26S subunit, non-ATPase, 9], PSME1 [proteasome (prosome, macropain) activator subunit 1 (PA28 alpha)], PSME2 [proteasome (prosome, macropain) activator subunit 2 (PA28 beta)], PSMG1 [proteasome (prosome, macropain) assembly chaperone 1], PSPH [phosphoserine phosphatase], PSPN [persephin], PSTPIP1 [proline-serine-threonine phosphatase interacting protein 1], PTAFR [platelet-activating factor receptor], PTCH1 [patched homolog 1 (Drosophila)], PTCH2 [patched homolog 2 (Drosophila)], PTEN [phosphatase and tensin homolog], PTF1A [pancreas specific transcription factor, 1a], PTGER1 [prostaglandin E receptor 1 (subtype EP1), 42 kDa], PTGER2 [prostaglandin E receptor 2 (subtype EP2), 53 kDa], PTGER3 [prostaglandin E receptor 3 (subtype EP3)], PTGER4 [prostaglandin E receptor 4 (subtype EP4)], PTGES [prostaglandin E synthase], PTGES2 [prostaglandin E synthase 2], PTGIR [prostaglandin 12 (prostacyclin) receptor (IP)], PTGS1 [prostaglandin-endoperoxide synthase 1 (prostaglandin G/H synthase and cyclooxygenase)], PTGS2 [prostaglandin-endoperoxide synthase 2 (prostaglandin G/H synthase and cyclooxygenase)], PTH [parathyroid hormone], PTH1R [parathyroid hormone 1 receptor], PTHLH [parathyroid hormone-like hormone], PTK2 [PTK2 protein tyrosine kinase 2], PTK2B [PTK2B protein tyrosine kinase 2 beta], PTK7 [PTK7 protein tyrosine kinase 7], PTN [pleiotrophin], PTPN1 [protein tyrosine phosphatase, non-receptor type 1], PTPN11 [protein tyrosine phosphatase, non-receptor type 11], PTPN13 [protein tyrosine phosphatase, non-receptor type 13 (APO-1/CD95 (Fas)-associated phosphatase)], PTPN18 [protein tyrosine phosphatase, non-receptor type 18 (brain-derived)], PTPN2 [protein tyrosine phosphatase, non-receptor type 2], PTPN22 [protein tyrosine phosphatase, non-receptor type 22 (lymphoid)], PTPN6 [protein tyrosine phosphatase, non-receptor type 6], PTPN7 [protein tyrosine phosphatase, non-receptor type 7], PTPRA [protein tyrosine phosphatase, receptor type, A], PTPRB [protein tyrosine phosphatase, receptor type, B], PTPRC [protein tyrosine phosphatase, receptor type, C], PTPRD [protein tyrosine phosphatase, receptor type, D], PTPRE [protein tyrosine phosphatase, receptor type, E], PTPRF [protein tyrosine phosphatase, receptor type, F], PTPRJ [protein tyrosine phosphatase, receptor type, J], PTPRK [protein tyrosine phosphatase, receptor type, K], PTPRM [protein tyrosine phosphatase, receptor type, M], PTPRO [protein tyrosine phosphatase, receptor type, O], PTPRS [protein tyrosine phosphatase, receptor type, S], PTPRT [protein tyrosine phosphatase, receptor type, T], PTPRU [protein tyrosine phosphatase, receptor type, U], PTPRZ1 [protein tyrosine phosphatase, receptor-type, Z polypeptide 1], PTS [6-pyruvoyltetrahydropterin synthase], PTTG1 [pituitary tumor-transforming 1], PVR [poliovirus receptor], PVRL1 [poliovirus receptor-related 1 (herpesvirus entry mediator C)], PWP2 [PWP2 periodic tryptophan protein homolog (yeast)], PXN [paxillin], PYCARD [PYD and CARD domain containing], PYGB [phosphorylase, glycogen; brain], PYGM [phosphorylase, glycogen, muscle], PYY [peptide YY], QDPR [quinoid dihydropteridine reductase], QKI [quaking homolog, KH domain RNA binding (mouse)], RAB11A [RAB11A, member RAS oncogene family], RAB11FIP5 [RAB11 family interacting protein 5 (class I)], RAB39B [RAB39B, member RAS oncogene family], RAB3A [RAB3A, member RAS oncogene family], RAB4A [RAB4A, member RAS oncogene family], RAB5A [RAB5A, member RAS oncogene family], RAB8A [RAB8A, member RAS oncogene family], RAB9A [RAB9A, member RAS oncogene family], RABEP1 [rabaptin, RAB GTPase binding effector protein 1], RABGEF1 [RAB guanine nucleotide exchange factor (GEF) 1], RAC1 [ras-related C3 botulinum toxin substrate 1 (rho family, small GTP binding protein Rac1)], RAC2 [ras-related C3 botulinum toxin substrate 2 (rho family, small GTP binding protein Rac2)], RAC3 [ras-related C3 botulinum toxin substrate 3 (rho family, small GTP binding protein Rac3)], RAD51 [RAD51 homolog (RecA homolog, E. coli) (S. cerevisiae)], RAF1 [v-raf-1 murine leukemia viral oncogene homolog 1], RAG1 [recombination activating gene 1], RAG2 [recombination activating gene 2], RAGE [renal tumor antigen], RALA [v-ral simian leukemia viral oncogene homolog A (ras related)], RALBP1 [ralA binding protein 1], RALGAPA2 [Ral GTPase activating protein, alpha subunit 2 (catalytic)], RALGAPB [Ral GTPase activating protein, beta subunit (non-catalytic)], RALGDS [ral guanine nucleotide dissociation stimulator], RAN [RAN, member RAS oncogene family], RAP1A [RAP1A, member of RAS oncogene family], RAP1B [RAP1B, member of RAS oncogene family], RAP1GAP [RAP1 GTPase activating protein], RAPGEF3 [Rap guanine nucleotide exchange factor (GEF) 3], RAPGEF4 [Rap guanine nucleotide exchange factor (GEF) 4], RAPH1 [Ras association (RalGDS/AF-6) and pleckstrin homology domains 1], RAPSN [receptor-associated protein of the synapse], RARA [retinoic acid receptor, alpha], RARB [retinoic acid receptor, beta], RARG [retinoic acid receptor, gamma], RARS [arginyl-tRNA synthetase], RASA1 [RAS p21 protein activator (GTPase activating protein) 1], RASA2 [RAS p21 protein activator 2], RASGRF1 [Ras protein-specific guanine nucleotide-releasing factor 1], RASGRP1 [RAS guanyl releasing protein 1 (calcium and DAG-regulated)], RASSF1 [Ras association (RalGDS/AF-6) domain family member 1], RASSF5 [Ras association (RalGDS/AF-6) domain family member 5], RB1 [retinoblastoma 1], RBBP4 [retinoblastoma binding protein 4], RBM11 [RNA binding motif protein 11], RBM4 [RNA binding motif protein 4], RBM45 [RNA binding motif protein 45], RBP4 [retinol binding protein 4, plasma], RBPJ [recombination signal binding protein for immunoglobulin kappa J region], RCAN1 [regulator of calcineurin 1], RCAN2 [regulator of calcineurin 2], RCAN3 [ROAN family member 3], RCOR1 [REST corepressor 1], RDX [radixin], REEP3 [receptor accessory protein 3], REG1A [regenerating islet-derived 1 alpha], RELA [v-rel reticuloendotheliosis viral oncogene homolog A (avian)], RELN [reelin], REN [renin], REPIN1 [replication initiator 1], REST [RE1-silencing transcription factor], RET [ret proto-oncogene], RETN [resistin], RFC1 [replication factor C (activator 1) 1, 145 kDa], RFC2 [replication factor C (activator 1) 2, 40 kDa], RFX1 [regulatory factor X, 1 (influences HLA class II expression)], RGMA [RGM domain family, member A], RGMB [RGM domain family, member B], RGS3 [regulator of G-protein signaling 3], RHD [Rh blood group, D antigen], RHEB [Ras homolog enriched in brain], RHO [rhodopsin], RHOA [ras homolog gene family, member A], RHOB [ras homolog gene family, member B], RHOC [ras homolog gene family, member C], RHOD [ras homolog gene family, member D], RHOG [ras homolog gene family, member G (rho G)], RHOH [ras homolog gene family, member H], RICTOR [RPTOR independent companion of MTOR, complex 2], RIMS3 [regulating synaptic membrane exocytosis 3], RIPK1 [receptor (TNFRSF)-interacting serine-threonine kinase 1], RIPK2 [receptor-interacting serine-threonine kinase 2], RNASE1 [ribonuclease, RNase A family, 1 (pancreatic)], RNASE3 [ribonuclease, RNase A family, 3 (eosinophil cationic protein)], RNASEL [ribonuclease L (2′ 5′-oligoisoadenylate synthetase-dependent)], RND1 [Rho family GTPase 1], RND2 [Rho family GTPase 2], RND3 [Rho family GTPase 3], RNF123 [ring finger protein 123], RNF128 [ring finger protein 128], RNF13 [ring finger protein 13], RNF135 [ring finger protein 135], RNF2 [ring finger protein 2], RNF6 [ring finger protein (C3H2C3 type) 6], RNH1 [ribonuclease/angiogenin inhibitor 1], RNPC3 [RNA-binding region (RNP1, RRM) containing 3], ROBO1 [roundabout, axon guidance receptor, homolog 1 (Drosophila)], ROBO2 [roundabout, axon guidance receptor, homolog 2 (Drosophila)], ROBO3 [roundabout, axon guidance receptor, homolog 3 (Drosophila)], ROBO4 [roundabout homolog 4, magic roundabout (Drosophila)], ROCK1 [Rho-associated, coiled-coil containing protein kinase 1], ROCK2 [Rho-associated, coiled-coil containing protein kinase 2], RPGR [retinitis pigmentosa GTPase regulator], RPGRIP1 [retinitis pigmentosa GTPase regulator interacting protein 1], RPGRIP1L [RPGRIP1-like], RPL10 [ribosomal protein L10], RPL24 [ribosomal protein L24], RPL5 [ribosomal protein L5], RPL7A [ribosomal protein L7a], RPLP0 [ribosomal protein, large, P0], RPS17 [ribosomal protein S17], RPS17P3 [ribosomal protein S17 pseudogene 3], RPS19 [ribosomal protein S19], RPS27A [ribosomal protein S27a], RPS6 [ribosomal protein S6], RPS6KA1 [ribosomal protein S6 kinase, 90 kDa, polypeptide 1], RPS6KA3 [ribosomal protein S6 kinase, 90 kDa, polypeptide 3], RPS6KA6 [ribosomal protein S6 kinase, 90 kDa, polypeptide 6], RPS6KB1 [ribosomal protein S6 kinase, 70 kDa, polypeptide 1], RRAS [related RAS viral (r-ras) oncogene homolog], RRAS2 [related RAS viral (r-ras) oncogene homolog 2], RRBP1 [ribosome binding protein 1 homolog 180 kDa (dog)], RRM1 [ribonucleotide reductase M1], RRM2 [ribonucleotide reductase M2], RRM2B [ribonucleotide reductase M2 B (TP53 inducible)], RTN4 [reticulon 4], RTN4R [reticulon 4 receptor], RUFY3 [RUN and FYVE domain containing 3], RUNX1 [runt-related transcription factor 1], RUNX1T1 [runt-related transcription factor 1; translocated to, 1 (cyclin D-related)], RUNX2 [runt-related transcription factor 2], RUNX3 [runt-related transcription factor 3], RUVBL2 [RuvB-like 2 (E. coli)], RXRA [retinoid X receptor, alpha], RYK [RYK receptor-like tyrosine kinase], RYR2 [ryanodine receptor 2 (cardiac)], RYR3 [ryanodine receptor 3], S100A1 [S100 calcium binding protein A1], S100A10 [S100 calcium binding protein A10], S100A12 [S100 calcium binding protein A12], S100A2 [S100 calcium binding protein A2], S100A4 [S100 calcium binding protein A4], S100A6 [S100 calcium binding protein A6], S100A7 [S100 calcium binding protein A7], S100A8 [S100 calcium binding protein A8], S100A9 [S100 calcium binding protein A9], S100B [S100 calcium binding protein B], SAA4 [serum amyloid A4, constitutive], SACS [spastic ataxia of Charlevoix-Saguenay (sacsin)], SAFB [scaffold attachment factor B], SAG [S-antigen; retina and pineal gland (arrestin)], SAMHD1 [SAM domain and HD domain 1], SATB2 [SATB homeobox 2], SBDS [Shwachman-Bodian-Diamond syndrome], SCARB1 [scavenger receptor class B, member 1], SCD [stearoyl-CoA desaturase (delta-9-desaturase)], SCD5 [stearoyl-CoA desaturase 5], SCG2 [secretogranin II], SCG5 [secretogranin V (7B2 protein)], SCGB1A1 [secretoglobin, family 1A, member 1 (uteroglobin)], SCN11A [sodium channel, voltage-gated, type XI, alpha subunit], SCN1A [sodium channel, voltage-gated, type I, alpha subunit], SCN2A [sodium channel, voltage-gated, type II, alpha subunit], SCN3A [sodium channel, voltage-gated, type III, alpha subunit], SCN5A [sodium channel, voltage-gated, type V, alpha subunit], SCN7A [sodium channel, voltage-gated, type VII, alpha], SCNN1B [sodium channel, nonvoltage-gated 1, beta], SCNN1G [sodium channel, nonvoltage-gated 1, gamma], SCP2 [sterol carrier protein 2], SCT [secretin], SCTR [secretin receptor], SCUBE1 [signal peptide, CUB domain, EGF-like 1], SDC2 [syndecan 2], SDC3 [syndecan 3], SDCBP [syndecan binding protein (syntenin)], SDHB [succinate dehydrogenase complex, subunit B, iron sulfur (Ip)], SDHD [succinate dehydrogenase complex, subunit D, integral membrane protein], SDS [serine dehydratase], SEC14L2 [SEC14-like 2 (S. cerevisiae)], SELE [selectin E], SELL [selectin L], SELP [selectin P (granule membrane protein 140 kDa, antigen CD62)], SELPLG [selectin P ligand], SEMA3A [sema domain, immunoglobulin domain (Ig), short basic domain, secreted, (semaphorin) 3A], SEMA3B [sema domain, immunoglobulin domain (Ig), short basic domain, secreted, (semaphorin) 3B], SEMA3C [sema domain, immunoglobulin domain (Ig), short basic domain, secreted, (semaphorin) 30], SEMA3D [sema domain, immunoglobulin domain (Ig), short basic domain, secreted, (semaphorin) 3D], SEMA3E [sema domain, immunoglobulin domain (Ig), short basic domain, secreted, (semaphorin) 3E], SEMA3F [sema domain, immunoglobulin domain (Ig), short basic domain, secreted, (semaphorin) 3F], SEMA3G [sema domain, immunoglobulin domain (Ig), short basic domain, secreted, (semaphorin) 3G], SEMA4A [sema domain, immunoglobulin domain (Ig), transmembrane domain (TM) and short cytoplasmic domain, (semaphorin) 4A], SEMA4B [sema domain, immunoglobulin domain (Ig), transmembrane domain (TM) and short cytoplasmic domain, (semaphorin) 4B], SEMA4C [sema domain, immunoglobulin domain (Ig), transmembrane domain (TM) and short cytoplasmic domain, (semaphorin) 40], SEMA4D [sema domain, immunoglobulin domain (Ig), transmembrane domain (TM) and short cytoplasmic domain, (semaphorin) 4D], SEMA4F [sema domain, immunoglobulin domain (Ig), transmembrane domain (TM) and short cytoplasmic domain, (semaphorin) 4F], SEMA4G [sema domain, immunoglobulin domain (Ig), transmembrane domain (TM) and short cytoplasmic domain, (semaphorin) 4G], SEMA5A [sema domain, seven thrombospondin repeats (type 1 and type 1-like), transmembrane domain (TM) and short cytoplasmic domain, (semaphorin) 5A], SEMA5B [sema domain, seven thrombospondin repeats (type 1 and type 1-like), transmembrane domain (TM) and short cytoplasmic domain, (semaphorin) 5B], SEMA6A [sema domain, transmembrane domain (TM), and cytoplasmic domain, (semaphorin) 6A], SEMA6B [sema domain, transmembrane domain (TM), and cytoplasmic domain, (semaphorin) 6B], SEMA6C [sema domain, transmembrane domain (TM), and cytoplasmic domain, (semaphorin) 60], SEMA6D [sema domain, transmembrane domain (TM), and cytoplasmic domain, (semaphorin) 6D], SEMA7A [semaphorin 7A, GPI membrane anchor (John Milton Hagen blood group)], SEPP1 [selenoprotein P, plasma, 1], SEPT2 [septin 2], SEPT4 [septin 4], SEPT5 [septin 5], SEPT6 [septin 6], SEPT7 [septin 7], SEPT9 [septin 9], SERPINA1 [serpin peptidase inhibitor, clade A (alpha-1 antiproteinase, antitrypsin), member 1], SERPINA3 [serpin peptidase inhibitor, clade A (alpha-1 antiproteinase, antitrypsin), member 3], SERPINA7 [serpin peptidase inhibitor, clade A (alpha-1 antiproteinase, antitrypsin), member 7], SERPINB1 [serpin peptidase inhibitor, clade B (ovalbumin), member 1], SERPINB2 [serpin peptidase inhibitor, clade B (ovalbumin), member 2], SERPINB6 [serpin peptidase inhibitor, clade B (ovalbumin), member 6], SERPINC1 [serpin peptidase inhibitor, clade C (antithrombin), member 1], SERPINE1 [serpin peptidase inhibitor, clade E (nexin, plasminogen activator inhibitor type 1), member 1], SERPINE2 [serpin peptidase inhibitor, clade E (nexin, plasminogen activator inhibitor type 1), member 2], SERPINF1 [serpin peptidase inhibitor, clade F (alpha-2 antiplasmin, pigment epithelium derived factor), member 1], SERPINH1 [serpin peptidase inhibitor, clade H (heat shock protein 47), member 1, (collagen binding protein 1)1, SERPINI1 [serpin peptidase inhibitor, clade I (neuroserpin), member 1], SET [SET nuclear oncogene], SETX [senataxin], SEZ6L2 [seizure related 6 homolog (mouse)-like 2], SFPQ [splicing factor proline/glutamine-rich (polypyrimidine tract binding protein associated)], SFRP1 [secreted frizzled-related protein 1], SFRP4 [secreted frizzled-related protein 4], SFRS15 [splicing factor, arginine/serine-rich 15], SFTPA1 [surfactant protein A1], SFTPB [surfactant protein B], SFTPC [surfactant protein C], SGCB [sarcoglycan, beta (43 kDa dystrophin-associated glycoprotein)], SGCE [sarcoglycan, epsilon], SGK1 [serum/glucocorticoid regulated kinase 1], SH2B1 [SH2B adaptor protein 1], SH2B3 [SH2B adaptor protein 3], SH2D1A [SH2 domain containing 1A], SH3BGR [SH3 domain binding glutamic acid-rich protein], SH3BGRL [SH3 domain binding glutamic acid-rich protein like], SH3BP1 [SH3-domain binding protein 1], SH3GL1P2 [SH3-domain GRB2-like 1 pseudogene 2], SH3GL3 [SH3-domain GRB2-like 3], SH3KBP1 [SH3-domain kinase binding protein 1], SH3PXD2A [SH3 and PX domains 2A], SHANK1 [SH3 and multiple ankyrin repeat domains 1], SHANK2 [SH3 and multiple ankyrin repeat domains 2], SHANK3 [SH3 and multiple ankyrin repeat domains 3], SHBG [sex hormone-binding globulin], SHC1 [SHC (Src homology 2 domain containing) transforming protein 1], SHC3 [SHC (Src homology 2 domain containing) transforming protein 3], SHH [sonic hedgehog homolog (Drosophila)], SHOC2 [soc-2 suppressor of clear homolog (C. elegans)], SI [sucrase-isomaltase (alpha-glucosidase)], SIAH1 [seven in absentia homolog 1 (Drosophila)], SIAH2 [seven in absentia homolog 2 (Drosophila)], SIGMAR1 [sigma non-opioid intracellular receptor 1], SILV [silver homolog (mouse)], SIM1 [single-minded homolog 1 (Drosophila)], SIM2 [single-minded homolog 2 (Drosophila)], SIP1 [survival of motor neuron protein interacting protein 1], SIRPA [signal-regulatory protein alpha], SIRT1 [sirtuin (silent mating type information regulation 2 homolog) 1 (S. cerevisiae)], SIRT4 [sirtuin (silent mating type information regulation 2 homolog) 4 (S. cerevisiae)], SIRT6 [sirtuin (silent mating type information regulation 2 homolog) 6 (S. cerevisiae)], SIX5 [SIX homeobox 5], SKI [v-ski sarcoma viral oncogene homolog (avian)], SKP2 [S-phase kinase-associated protein 2 (p45)], SLAMF6 [SLAM family member 6], SLC10A1 [solute carrier family 10 (sodium/bile acid cotransporter family), member 1], SLC11A2 [solute carrier family 11 (proton-coupled divalent metal ion transporters), member 2], SLC12A1 [solute carrier family 12 (sodium/potassium/chloride transporters), member 1], SLC12A2 [solute carrier family 12 (sodium/potassium/chloride transporters), member 2], SLC12A3 [solute carrier family 12 (sodium/chloride transporters), member 3], SLC12A5 [solute carrier family 12 (potassium/chloride transporter), member 5], SLC12A6 [solute carrier family 12 (potassium/chloride transporters), member 6], SLC13A1 [solute carrier family 13 (sodium/sulfate symporters), member 1], SLC15A1 [solute carrier family 15 (oligopeptide transporter), member 1], SLC16A2 [solute carrier family 16, member 2 (monocarboxylic acid transporter 8)], SLC17A5 [solute carrier family 17 (anion/sugar transporter), member 5], SLC17A7 [solute carrier family 17 (sodium-dependent inorganic phosphate cotransporter), member 7], SLC18A2 [solute carrier family 18 (vesicular monoamine), member 2], SLC18A3 [solute carrier family 18 (vesicular acetylcholine), member 3], SLC19A1 [solute carrier family 19 (folate transporter), member 1], SLC19A2 [solute carrier family 19 (thiamine transporter), member 2], SLC1A1 [solute carrier family 1 (neuronal/epithelial high affinity glutamate transporter, system Xag), member 1], SLC1A2 [solute carrier family 1 (glial high affinity glutamate transporter), member 2], SLC1A3 [solute carrier family 1 (glial high affinity glutamate transporter), member 3], SLC22A2 [solute carrier family 22 (organic cation transporter), member 2], SLC25A12 [solute carrier family 25 (mitochondrial carrier, Aralar), member 12], SLC25A13 [solute carrier family 25, member 13 (citrin)], SLC25A20 [solute carrier family 25 (carnitine/acylcarnitine translocase), member 20], SLC25A3 [solute carrier family 25 (mitochondrial carrier; phosphate carrier), member 3], SLC26A3 [solute carrier family 26, member 3], SLC27A1 [solute carrier family 27 (fatty acid transporter), member 1], SLC29A1 [solute carrier family 29 (nucleoside transporters), member 1], SLC2A1 [solute carrier family 2 (facilitated glucose transporter), member 1], SLC2A13 [solute carrier family 2 (facilitated glucose transporter), member 13], SLC2A2 [solute carrier family 2 (facilitated glucose transporter), member 2], SLC2A3 [solute carrier family 2 (facilitated glucose transporter), member 3], SLC2A4 [solute carrier family 2 (facilitated glucose transporter), member 4], SLC30A3 [solute carrier family 30 (zinc transporter), member 3], SLC30A4 [solute carrier family 30 (zinc transporter), member 4], SLC30A8 [solute carrier family 30 (zinc transporter), member 8], SLC31A1 [solute carrier family 31 (copper transporters), member 1], SLC32A1 [solute carrier family 32 (GABA vesicular transporter), member 1], SLC34A1 [solute carrier family 34 (sodium phosphate), member 1], SLC38A3 [solute carrier family 38, member 3], SLC39A2 [solute carrier family 39 (zinc transporter), member 2], SLC39A3 [solute carrier family 39 (zinc transporter), member 3], SLC40A1 [solute carrier family 40 (iron-regulated transporter), member 1], SLC4A11 [solute carrier family 4, sodium borate transporter, member 11], SLC5A3 [solute carrier family 5 (sodium/myo-inositol cotransporter), member 3], SLC5A8 [solute carrier family 5 (iodide transporter), member 8], SLC6A1 [solute carrier family 6 (neurotransmitter transporter, GABA), member 1], SLC6A14 [solute carrier family 6 (amino acid transporter), member 14], SLC6A2 [solute carrier family 6 (neurotransmitter transporter, noradrenalin), member 2], SLC6A3 [solute carrier family 6 (neurotransmitter transporter, dopamine), member 3], SLC6A4 [solute carrier family 6 (neurotransmitter transporter, serotonin), member 4], SLC6A8 [solute carrier family 6 (neurotransmitter transporter, creatine), member 8], SLC7A14 [solute carrier family 7 (cationic amino acid transporter, y+ system), member 14], SLC7A5 [solute carrier family 7 (cationic amino acid transporter, y+ system), member 5], SLC9A2 [solute carrier family 9 (sodium/hydrogen exchanger), member 2], SLC9A3 [solute carrier family 9 (sodium/hydrogen exchanger), member 3], SLC9A3R1 [solute carrier family 9 (sodium/hydrogen exchanger), member 3 regulator 1], SLC9A3R2 [solute carrier family 9 (sodium/hydrogen exchanger), member 3 regulator 2], SLC9A6 [solute carrier family 9 (sodium/hydrogen exchanger), member 6], SLIT1 [slit homolog 1 (Drosophila)], SLIT2 [slit homolog 2 (Drosophila)], SLIT3 [slit homolog 3 (Drosophila)], SLITRK1 [SLIT and NTRK-like family, member 1], SLN [sarcolipin], SLPI [secretory leukocyte peptidase inhibitor], SMAD1 [SMAD family member 1], SMAD2 [SMAD family member 2], SMAD3 [SMAD family member 3], SMAD4 [SMAD family member 4], SMAD6 [SMAD family member 6], SMAD7 [SMAD family member 7], SMARCA1 [SWI/SNF related, matrix associated, actin dependent regulator of chromatin, subfamily a, member 1], SMARCA2 [SWI/SNF related, matrix associated, actin dependent regulator of chromatin, subfamily a, member 2], SMARCA4 [SWI/SNF related, matrix associated, actin dependent regulator of chromatin, subfamily a, member 4], SMARCA5 [SWI/SNF related, matrix associated, actin dependent regulator of chromatin, subfamily a, member 5], SMARCB1 [SWI/SNF related, matrix associated, actin dependent regulator of chromatin, subfamily b, member 1], SMARCC1 [SWI/SNF related, matrix associated, actin dependent regulator of chromatin, subfamily c, member 1], SMARCC2 [SWI/SNF related, matrix associated, actin dependent regulator of chromatin, subfamily c, member 2], SMARCD1 [SWI/SNF related, matrix associated, actin dependent regulator of chromatin, subfamily d, member 1], SMARCD3 [SWI/SNF related, matrix associated, actin dependent regulator of chromatin, subfamily d, member 3], SMARCE1 [SWI/SNF related, matrix associated, actin dependent regulator of chromatin, subfamily e, member 1], SMG1 [SMG1 homolog, phosphatidylinositol 3-kinase-related kinase (C. elegans)], SMN1 [survival of motor neuron 1, telomeric], SMO [smoothened homolog (Drosophila)], SMPD1 [sphingomyelin phosphodiesterase 1, acid lysosomal], SMS [spermine synthase], SNAI2 [snail homolog 2 (Drosophila)], SNAP25 [synaptosomal-associated protein, 25 kDa], SNCA [synuclein, alpha (non A4 component of amyloid precursor)], SNCAIP [synuclein, alpha interacting protein], SNOB [synuclein, beta], SNCG [synuclein, gamma (breast cancer-specific protein 1)], SNRPA [small nuclear ribonucleoprotein polypeptide A], SNRPN [small nuclear ribonucleoprotein polypeptide N], SNTG2 [syntrophin, gamma 2], SNURF [SNRPN upstream reading frame], SOAT1 [sterol O-acyltransferase 1], SOCS1 [suppressor of cytokine signaling 1], SOCS3 [suppressor of cytokine signaling 3], SOD1 [superoxide dismutase 1, soluble], SOD2 [superoxide dismutase 2, mitochondrial], SORBS3 [sorbin and SH3 domain containing 3], SORL1 [sortilin-related receptor, L(DLR class) A repeats-containing], SORT1 [sortilin 1], SOS1 [son of sevenless homolog 1 (Drosophila)], SOS2 [son of sevenless homolog 2 (Drosophila)], SOSTDC1 [sclerostin domain containing 1], SOX1 [SRY (sex determining region Y)-box 1], SOX10 [SRY (sex determining region Y)-box 10], SOX18 [SRY (sex determining region Y)-box 18], SOX2 [SRY (sex determining region Y)-box 2], SOX3 [SRY (sex determining region Y)-box 3], SOX9 [SRY (sex determining region Y)-box 9], SP1 [Sp1 transcription factor], SP3 [Sp3 transcription factor], SPANXB1 [SPANX family, member B1], SPANXC [SPANX family, member C], SPARC [secreted protein, acidic, cysteine-rich (osteonectin)], SPARCL1 [SPARC-like 1 (hevin)], SPAST [spastin], SPHK1 [sphingosine kinase 1], SPINK1 [serine peptidase inhibitor, Kazal type 1], SPINT2 [serine peptidase inhibitor, Kunitz type, 2], SPN [sialophorin], SPNS2 [spinster homolog 2 (Drosophila)], SPON2 [spondin 2, extracellular matrix protein], SPP1 [secreted phosphoprotein 1], SPRED2 [sprouty-related, EVH1 domain containing 2], SPRY2 [sprouty homolog 2 (Drosophila)], SPTA1 [spectrin, alpha, erythrocytic 1 (elliptocytosis 2)], SPTAN1 [spectrin, alpha, non-erythrocytic 1 (alpha-fodrin)], SPTB [spectrin, beta, erythrocytic], SPTBN1 [spectrin, beta, non-erythrocytic 1], SRC [v-src sarcoma (Schmidt-Ruppin A-2) viral oncogene homolog (avian)], SRCRB4D [scavenger receptor cysteine rich domain containing, group B (4 domains)], SRD5A1 [steroid-5-alpha-reductase, alpha polypeptide 1 (3-oxo-5 alpha-steroid delta 4-dehydrogenase alpha 1)], SREBF1 [sterol regulatory element binding transcription factor 1], SREBF2 [sterol regulatory element binding transcription factor 2], SRF [serum response factor (c-fos serum response element-binding transcription factor)], SRGAP1 [SLIT-ROBO Rho GTPase activating protein 1], SRGAP2 [SLIT-ROBO Rho GTPase activating protein 2], SRGAP3 [SLIT-ROBO Rho GTPase activating protein 3], SRPX [sushi-repeat-containing protein, X-linked], SRY [sex determining region Y], SSB [Sjogren syndrome antigen B (autoantigen La)], SSH1 [slingshot homolog 1 (Drosophila)], SSRP1 [structure specific recognition protein 1], SST [somatostatin], SSTR1 [somatostatin receptor 1], SSTR2 [somatostatin receptor 2], SSTR3 [somatostatin receptor 3], SSTR4 [somatostatin receptor 4], SSTR5 [somatostatin receptor 5], ST13 [suppression of tumorigenicity 13 (colon carcinoma) (Hsp70 interacting protein)], ST14 [suppression of tumorigenicity 14 (colon carcinoma)], ST6GAL1 [ST6 beta-galactosamide alpha-2 [6-sialyltranferase 1], ST7 [suppression of tumorigenicity 7], STAG2 [stromal antigen 2], STAG3 [stromal antigen 3], STAR [steroidogenic acute regulatory protein], STAT1 [signal transducer and activator of transcription 1, 91 kDa], STAT2 [signal transducer and activator of transcription 2, 113 kDa], STAT3 [signal transducer and activator of transcription 3 (acute-phase response factor)], STAT4 [signal transducer and activator of transcription 4], STAT5A [signal transducer and activator of transcription 5A], STAT5B [signal transducer and activator of transcription 5B], STAT6 [signal transducer and activator of transcription 6, interleukin-4 induced], STATH [statherin], STC1 [stanniocalcin 1], STIL [SCL/TAL1 interrupting locus], STIM1 [stromal interaction molecule 1], STK11 [serine/threonine kinase 11], STK24 [serine/threonine kinase 24 (STE20 homolog, yeast)], STK36 [serine/threonine kinase 36, fused homolog (Drosophila)], STK38 [serine/threonine kinase 38], STK38L [serine/threonine kinase 38 like], STK39 [serine threonine kinase 39 (STE20/SPS1 homolog, yeast)], STMN1 [stathmin 1], STMN2 [stathmin-like 2], STMN3 [stathmin-like 3], STMN4 [stathmin-like 4], STOML1 [stomatin (EPB72)-like 1], STS [steroid sulfatase (microsomal), isozyme S], STUB1 [STIP1 homology and U-box containing protein 1], STX1A [syntaxin 1A (brain)], STX3 [syntaxin 3], STYX [serine/threonine/tyrosine interacting protein], SUFU [suppressor of fused homolog (Drosophila)], SULT2A1 [sulfotransferase family, cytosolic, 2A, dehydroepiandrosterone (DHEA)-preferring, member 1], SUMO1 [SMT3 suppressor of mif two 3 homolog 1 (S. cerevisiae)], SUMO3 [SMT3 suppressor of mif two 3 homolog 3 (S. cerevisiae)], SUN1 [Sad1 and UNC84 domain containing 1], SUN2 [Sad1 and UNC84 domain containing 2], SUPT16H [suppressor of Ty 16 homolog (S. cerevisiae)], SUZ12P [suppressor of zeste 12 homolog pseudogene], SV2A [synaptic vesicle glycoprotein 2A], SYK [spleen tyrosine kinase], SYN1 [synapsin I], SYN2 [synapsin II], SYN3 [synapsin III], SYNGAP1 [synaptic Ras GTPase activating protein 1 homolog (rat)], SYNJ1 [synaptojanin 1], SYNPO2 [synaptopodin 2], SYP [synaptophysin], SYT1 [synaptotagmin I], TAC1 [tachykinin, precursor 1], TAC3 [tachykinin 3], TACR1 [tachykinin receptor 1], TAF1 [TAF1 RNA polymerase II, TATA box binding protein (TBP)-associated factor, 250 kDa], TAF6 [TAF6 RNA polymerase II, TATA box binding protein (TBP)-associated factor, 80 kDa], TAGAP [T-cell activation RhoGTPase activating protein], TAGLN [transgelin], TAGLN3 [transgelin 3], TAOK2 [TAO kinase 2], TAP1 [transporter 1, ATP-binding cassette, sub-family B (MDR/TAP)], TAP2 [transporter 2, ATP-binding cassette, sub-family B (MDR/TAP)], TAPBP [TAP binding protein (tapasin)], TARDBP [TAR DNA binding protein], TARP [TCR gamma alternate reading frame protein], TAS2R1 [taste receptor, type 2, member 1], TAT [tyrosine aminotransferase], TBC1D4 [TBC1 domain family, member 4], TBCB [tubulin folding cofactor B], TBCD [tubulin folding cofactor D], TBCE [tubulin folding cofactor E], TBL1Y [transducin (beta)-like 1, Y-linked], TBL2 [transducin (beta)-like 2], TBP [TATA box binding protein], TBPL2 [TATA box binding protein like 2], TBR1 [T-box, brain, 1], TBX1 [T-box 1], TBX21 [T-box 21], TBXA2R [thromboxane A2 receptor], TBXAS1 [thromboxane A synthase 1 (platelet)], TCEB3 [transcription elongation factor B (SIII), polypeptide 3 (110 kDa, elongin A)], TCF12 [transcription factor 12], TCF19 [transcription factor 19], TCF4 [transcription factor 4], TCF7 [transcription factor 7 (T-cell specific, HMG-box)], TCF7L2 [transcription factor 7-like 2 (T-cell specific, HMG-box)], TCHH [trichohyalin], TCN1 [transcobalamin I (vitamin B12 binding protein, R binder family)], TCN2 [transcobalamin II; macrocytic anemia], TCP1 [t-complex 1], TDO2 [tryptophan 2 [3-dioxygenase], TDRD3 [tudor domain containing 3], TEAD2 [TEA domain family member 2], TEAD4 [TEA domain family member 4], TEK [TEK tyrosine kinase, endothelial], TERF1 [telomeric repeat binding factor (NIMA-interacting) 1], TERF2 [telomeric repeat binding factor 2], TERT [telomerase reverse transcriptase], TET2 [tet oncogene family member 2], TF [transferrin], TFAM [transcription factor A, mitochondrial], TFAP2A [transcription factor AP-2 alpha (activating enhancer binding protein 2 alpha)], TFCP2 [transcription factor CP2], TFF1 [trefoil factor 1], TFF2 [trefoil factor 2], TFF3 [trefoil factor 3 (intestinal)], TFPI [tissue factor pathway inhibitor (lipoprotein-associated coagulation inhibitor)], TFPI2 [tissue factor pathway inhibitor 2], TFRC [transferrin receptor (p90, CD71)], TG [thyroglobulin], TGFα [transforming growth factor, alpha], TGFB1 [transforming growth factor, beta 1], TGFB1I1 [transforming growth factor beta 1 induced transcript 1], TGFB2 [transforming growth factor, beta 2], TGFB3 [transforming growth factor, beta 3], TGFBR1 [transforming growth factor, beta receptor 1], TGFBR2 [transforming growth factor, beta receptor II (70/80 kDa)], TGFBR3 [transforming growth factor, beta receptor III], TGIF1 [TGFB-induced factor homeobox 1], TGM2 [transglutaminase 2 (C polypeptide, protein-glutamine-gamma-glutamyltransferase)], TH [tyrosine hydroxylase], THAP1 [THAP domain containing, apoptosis associated protein 1], THBD [thrombomodulin], THBS1 [thrombospondin 1], THBS2 [thrombospondin 2], THBS4 [thrombospondin 4], THEM4 [thioesterase superfamily member 4], THPO [thrombopoietin], THRA [thyroid hormone receptor, alpha (erythroblastic leukemia viral (v-erb-a) oncogene homolog, avian)], THY1 [Thy-1 cell surface antigen], TIAM1 [T-cell lymphoma invasion and metastasis 1], TIAM2 [T-cell lymphoma invasion and metastasis 2], TIMP1 [TIMP metallopeptidase inhibitor 1], TIMP2 [TIMP metallopeptidase inhibitor 2], TIMP3 [TIMP metallopeptidase inhibitor 3], TINF2 [TERF1 (TRF1)-interacting nuclear factor 2], TJP1 [tight junction protein 1 (zona occludens 1)], TJP2 [tight junction protein 2 (zona occludens 2)], TK1 [thymidine kinase 1, soluble], TKT [transketolase], TLE1 [transducin-like enhancer of split 1 (E(sp1) homolog, Drosophila)], TLR1 [toll-like receptor 1], TLR2 [toll-like receptor 2], TLR3 [toll-like receptor 3], TLR4 [toll-like receptor 4], TLR5 [toll-like receptor 5], TLR7 [toll-like receptor 7], TLR8 [toll-like receptor 8], TLR9 [toll-like receptor 9], TLX3 [T-cell leukemia homeobox 3], TMEFF1 [transmembrane protein with EGF-like and two follistatin-like domains 1], TMEM100 [transmembrane protein 100], TMEM216 [transmembrane protein 216], TMEM50B [transmembrane protein 50B], TMEM67 [transmembrane protein 67], TMEM70 [transmembrane protein 70], TMEM87A [transmembrane protein 87A], TMOD2 [tropomodulin 2 (neuronal)], TMOD4 [tropomodulin 4 (muscle)], TMPRSS11A [transmembrane protease, serine 11A], TMPRSS15 [transmembrane protease, serine 15], TMPRSS2 [transmembrane protease, serine 2], TNC [tenascin C], TNF [tumor necrosis factor (TNF superfamily, member 2)], TNFAIP3 [tumor necrosis factor, alpha-induced protein 3], TNFRSF10A [tumor necrosis factor receptor superfamily, member 10a], TNFRSF10B [tumor necrosis factor receptor superfamily, member 10b], TNFRSF10C [tumor necrosis factor receptor superfamily, member 10c, decoy without an intracellular domain], TNFRSF10D [tumor necrosis factor receptor superfamily, member 10d, decoy with truncated death domain], TNFRSF11B [tumor necrosis factor receptor superfamily, member 11b], TNFRSF18 [tumor necrosis factor receptor superfamily, member 18], TNFRSF19 [tumor necrosis factor receptor superfamily, member 19], TNFRSF1A [tumor necrosis factor receptor superfamily, member 1A], TNFRSF1B [tumor necrosis factor receptor superfamily, member 1B], TNFRSF25 [tumor necrosis factor receptor superfamily, member 25], TNFRSF8 [tumor necrosis factor receptor superfamily, member 8], TNFSF10 [tumor necrosis factor (ligand) superfamily, member 10], TNFSF11 [tumor necrosis factor (ligand) superfamily, member 11], TNFSF13 [tumor necrosis factor (ligand) superfamily, member 13], TNFSF13B [tumor necrosis factor (ligand) superfamily, member 13b], TNFSF4 [tumor necrosis factor (ligand) superfamily, member 4], TNK2 [tyrosine kinase, non-receptor, 2], TNNI3 [troponin I type 3 (cardiac)], TNNT1 [troponin T type 1 (skeletal, slow)], TNNT2 [troponin T type 2 (cardiac)], TNR [tenascin R (restrictin, janusin)], TNS1 [tensin 1], TNS3 [tensin 3], TNXB [tenascin XB], TOLLIP [toll interacting protein], TOP1 [topoisomerase (DNA) I], TOP2A [topoisomerase (DNA) II alpha 170 kDa], TOP2B [topoisomerase (DNA) II beta 180 kDa], TOR1A [torsin family 1, member A (torsin A)], TP53 [tumor protein p53], TP53BP1 [tumor protein p53 binding protein 1], TP63 [tumor protein p63], TP73 [tumor protein p73], TPH1 [tryptophan hydroxylase 1], TPH2 [tryptophan hydroxylase 2], TPI1 [triosephosphate isomerase 1], TPO [thyroid peroxidase], TPT1 [tumor protein, translationally-controlled 1], TPTE [transmembrane phosphatase with tensin homology], TRADD [TNFRSF1A-associated via death domain], TRAF2 [TNF receptor-associated factor 2], TRAF3 [TNF receptor-associated factor 3], TRAF6 [TNF receptor-associated factor 6], TRAP1 [TNF receptor-associated protein 1], TREM1 [triggering receptor expressed on myeloid cells 1], TRH [thyrotropin-releasing hormone], TRIM21 [tripartite motif-containing 21], TRIM22 [tripartite motif-containing 22], TRIM26 [tripartite motif-containing 26], TRIM27 [tripartite motif-containing 27], TRIM50 [tripartite motif-containing 50], TRIO [triple functional domain (PTPRF interacting)], TRPA1 [transient receptor potential cation channel, subfamily A, member 1], TRPC1 [transient receptor potential cation channel, subfamily C, member 1], TRPC5 [transient receptor potential cation channel, subfamily C, member 5], TRPC6 [transient receptor potential cation channel, subfamily C, member 6], TRPM1 [transient receptor potential cation channel, subfamily M, member 1], TRPV1 [transient receptor potential cation channel, subfamily V, member 1], TRPV2 [transient receptor potential cation channel, subfamily V, member 2], TRRAP [transformation/transcription domain-associated protein], TSC1 [tuberous sclerosis 1], TSC2 [tuberous sclerosis 2], TSC22D3 [TSC22 domain family, member 3], TSG101 [tumor susceptibility gene 101], TSHR [thyroid stimulating hormone receptor], TSN [translin], TSPAN12 [tetraspanin 12], TSPAN7 [tetraspanin 7], TSPO [translocator protein (18 kDa)], TTC3 [tetratricopeptide repeat domain 3], TTF1 [transcription termination factor, RNA polymerase I], TTF2 [transcription termination factor, RNA polymerase II], TTN [titin], TTPA [tocopherol (alpha) transfer protein], TTR [transthyretin], TUB [tubby homolog (mouse)], TUBA1A [tubulin, alpha 1a], TUBA1B [tubulin, alpha 1b], TUBA1C [tubulin, alpha 1c], TUBA3C [tubulin, alpha 3c], TUBA3D [tubulin, alpha 3d], TUBA4A [tubulin, alpha 4a], TUBA8 [tubulin, alpha 8], TUBB [tubulin, beta], TUBB1 [tubulin, beta 1], TUBB2A [tubulin, beta 2A], TUBB2B [tubulin, beta 2B], TUBB2C [tubulin, beta 20], TUBB3 [tubulin, beta 3], TUBB4 [tubulin, beta 4], TUBB4Q [tubulin, beta polypeptide 4, member Q], TUBB6 [tubulin, beta 6], TUBGCP5 [tubulin, gamma complex associated protein 5], TUFM [Tu translation elongation factor, mitochondrial], TUSC3 [tumor suppressor candidate 3], TWIST1 [twist homolog 1 (Drosophila)], TXN [thioredoxin], TXNIP [thioredoxin interacting protein], TXNRD1 [thioredoxin reductase 1], TXNRD2 [thioredoxin reductase 2], TYK2 [tyrosine kinase 2], TYMP [thymidine phosphorylase], TYMS [thymidylate synthetase], TYR [tyrosinase (oculocutaneous albinism IA)], TYRO3 [TYRO3 protein tyrosine kinase], TYROBP [TYRO protein tyrosine kinase binding protein], TYRP1 [tyrosinase-related protein 1], U2AF1 [U2 small nuclear RNA auxiliary factor 1], UBA1 [ubiquitin-like modifier activating enzyme 1], UBA52 [ubiquitin A-52 residue ribosomal protein fusion product 1], UBB [ubiquitin B], UBC [ubiquitin C], UBE2A [ubiquitin-conjugating enzyme E2A (RAD6 homolog)], UBE2C [ubiquitin-conjugating enzyme E20], UBE2D2 [ubiquitin-conjugating enzyme E2D 2 (UBC4/5 homolog, yeast)], UBE2H [ubiquitin-conjugating enzyme E2H (UBC8 homolog, yeast)], UBE2I [ubiquitin-conjugating enzyme E2I (UBC9 homolog, yeast)], UBE3A [ubiquitin protein ligase E3A], UBL5 [ubiquitin-like 5], UCHL1 [ubiquitin carboxyl-terminal esterase L1 (ubiquitin thiolesterase)], UCN [urocortin], UCP1 [uncoupling protein 1 (mitochondrial, proton carrier)], UCP2 [uncoupling protein 2 (mitochondrial, proton carrier)], UCP3 [uncoupling protein 3 (mitochondrial, proton carrier)], UGT1A1 [UDP glucuronosyltransferase 1 family, polypeptide A1], UGT1A3 [UDP glucuronosyltransferase 1 family, polypeptide A3], ULK1 [unc-51-like kinase 1 (C. elegans)], UNC5A [unc-5 homolog A (C. elegans)], UNC5B [unc-5 homolog B (C. elegans)], UNC5C [unc-5 homolog C (C. elegans)], UNC5D [unc-5 homolog D (C. elegans)], UNG [uracil-DNA glycosylase], UPF3B [UPF3 regulator of nonsense transcripts homolog B (yeast)], UPK3B [uroplakin 3B], UPP2 [uridine phosphorylase 2], UQCRC1 [ubiquinol-cytochrome c reductase core protein I], USF1 [upstream transcription factor 1], USF2 [upstream transcription factor 2, c-fos interacting], USH2A [Usher syndrome 2A (autosomal recessive, mild)], USP1 [ubiquitin specific peptidase 1], USP15 [ubiquitin specific peptidase 15], USP25 [ubiquitin specific peptidase 25], USP29 [ubiquitin specific peptidase 29], USP33 [ubiquitin specific peptidase 33], USP4 [ubiquitin specific peptidase 4 (proto-oncogene)], USP5 [ubiquitin specific peptidase 5 (isopeptidase T)], USP9X [ubiquitin specific peptidase 9, X-linked], USP9Y [ubiquitin specific peptidase 9, Y-linked], UTRN [utrophin], UXT [ubiquitously-expressed transcript], VAMP7 [vesicle-associated membrane protein 7], VASP [vasodilator-stimulated phosphoprotein], VAV1 [vav 1 guanine nucleotide exchange factor], VAV2 [vav 2 guanine nucleotide exchange factor], VAX1 [ventral anterior homeobox 1], VCAM1 [vascular cell adhesion molecule 1], VCL [vinculin], VDAC1 [voltage-dependent anion channel 1], VDAC2 [voltage-dependent anion channel 2], VDR [vitamin D (1 [25-dihydroxyvitamin D3) receptor], VEGFA [vascular endothelial growth factor A], VEGFB [vascular endothelial growth factor B], VEGFC [vascular endothelial growth factor C], VGF [VGF nerve growth factor inducible], VHL [von Hippel-Lindau tumor suppressor], VIM [vimentin], VIP [vasoactive intestinal peptide], VIPR1 [vasoactive intestinal peptide receptor 1], VIPR2 [vasoactive intestinal peptide receptor 2], VKORC1 [vitamin K epoxide reductase complex, subunit 1], VLDLR [very low density lipoprotein receptor], VPS29 [vacuolar protein sorting 29 homolog (S. cerevisiae)], VSIG4 [V-set and immunoglobulin domain containing 4], VSX1 [visual system homeobox 1], VTN [vitronectin], VWC2 [von Willebrand factor C domain containing 2], VWF [von Willebrand factor], WAS [Wiskott-Aldrich syndrome (eczema-thrombocytopenia)], WASF1 [WAS protein family, member 1], WASF2 [WAS protein family, member 2], WASL [Wiskott-Aldrich syndrome-like], WBSCR16 [Williams-Beuren syndrome chromosome region 16], WBSCR17 [Williams-Beuren syndrome chromosome region 17], WBSCR22 [Williams Beuren syndrome chromosome region 22], WBSCR27 [Williams Beuren syndrome chromosome region 27], WBSCR28 [Williams-Beuren syndrome chromosome region 28], WDR4 [WD repeat domain 4], WEE1 [WEE1 homolog (S. pombe)], WHAMM [WAS protein homolog associated with actin, golgi membranes and microtubules], WIPF1 [WAS/WASL interacting protein family, member 1], WIPF3 [WAS/WASL interacting protein family, member 3], WNK3 [WNK lysine deficient protein kinase 3], WNT1 [wingless-type MMTV integration site family, member 1], WNT10A [wingless-type MMTV integration site family, member 10A], WNT10B [wingless-type MMTV integration site family, member 10B], WNT11 [wingless-type MMTV integration site family, member 11], WNT16 [wingless-type MMTV integration site family, member 16], WNT2 [wingless-type MMTV integration site family member 2], WNT2B [wingless-type MMTV integration site family, member 2B], WNT3 [wingless-type MMTV integration site family, member 3], WNT3A [wingless-type MMTV integration site family, member 3A], WNT4 [wingless-type MMTV integration site family, member 4], WNT5A [wingless-type MMTV integration site family, member 5A], WNT5B [wingless-type MMTV integration site family, member 5B], WNT6 [wingless-type MMTV integration site family, member 6], WNT7A [wingless-type MMTV integration site family, member 7A], WNT7B [wingless-type MMTV integration site family, member 7B], WNT8A [wingless-type MMTV integration site family, member 8A], WNT8B [wingless-type MMTV integration site family, member 8B], WNT9A [wingless-type MMTV integration site family, member 9A], WNT9B [wingless-type MMTV integration site family, member 9B], WRB [tryptophan rich basic protein], WRN [Werner syndrome, RecQ helicase-like], WT1 [Wilms tumor 1], XBP1 [X-box binding protein 1], XCL1 [chemokine (C motif) ligand 1], XDH [xanthine dehydrogenase], XIAP [X-linked inhibitor of apoptosis], XIRP2 [xin actin-binding repeat containing 2], XPC [xeroderma pigmentosum, complementation group C], XRCC1 [X-ray repair complementing defective repair in Chinese hamster cells 1], XRCC5 [X-ray repair complementing defective repair in Chinese hamster cells 5 (double-strand-break rejoining)], XRCC6 [X-ray repair complementing defective repair in Chinese hamster cells 6], XRN1 [5′-3′ exoribonuclease 1], YBX1 [Y box binding protein 1], YWHAB [tyrosine 3-monooxygenase/tryptophan 5-monooxygenase activation protein, beta polypeptide], YWHAE [tyrosine 3-monooxygenase/tryptophan 5-monooxygenase activation protein, epsilon polypeptide], YWHAG [tyrosine 3-monooxygenase/tryptophan 5-monooxygenase activation protein, gamma polypeptide], YWHAQ [tyrosine 3-monooxygenase/tryptophan 5-monooxygenase activation protein, theta polypeptide], YWHAZ [tyrosine 3-monooxygenase/tryptophan 5-monooxygenase activation protein, zeta polypeptide], ZAP70 [zeta-chain (TCR) associated protein kinase 70 kDa], ZBTB16 [zinc finger and BTB domain containing 16], ZBTB33 [zinc finger and BTB domain containing 33], ZC3H12A [zinc finger CCCH-type containing 12A], ZEB1 [zinc finger E-box binding homeobox 1], ZEB2 [zinc finger E-box binding homeobox 2], ZFP161 [zinc finger protein 161 homolog (mouse)], ZFP36 [zinc finger protein 36, C3H type, homolog (mouse)], ZFP42 [zinc finger protein 42 homolog (mouse)], ZFP57 [zinc finger protein 57 homolog (mouse)], ZFPM1 [zinc finger protein, multitype 1], ZFPM2 [zinc finger protein, multitype 2], ZFY [zinc finger protein, Y-linked], ZFYVE9 [zinc finger, FYVE domain containing 9], ZIC1 [Zic family member 1 (odd-paired homolog, Drosophila)], ZIC2 [Zic family member 2 (odd-paired homolog, Drosophila)], ZIC3 [Zic family member 3 (odd-paired homolog, Drosophila)], ZMPSTE24 [zinc metallopeptidase (STE24 homolog, S. cerevisiae)], ZNF148 [zinc finger protein 148], ZNF184 [zinc finger protein 184], ZNF225 [zinc finger protein 225], ZNF256 [zinc finger protein 256], ZNF333 [zinc finger protein 333], ZNF385B [zinc finger protein 385B], ZNF44 [zinc finger protein 44], ZNF521 [zinc finger protein 521], ZNF673 [zinc finger family member 673], ZNF79 [zinc finger protein 79], ZNF84 [zinc finger protein 84], ZW10 [ZW10, kinetochore associated, homolog (Drosophila)], and ZYX [zyxin].

Preferred neurodevelopmental genes may include BMP4 (bone morphogenetic protein 4); CHRD (chordin); NOG (noggin); WNT2 (wingless-type MMTV integration site family member 2); WNT2B (wingless-type MMTV integration site family, member 2B); WNT3A (wingless-type MMTV integration site family, member 3A); WNT4(wingless-type MMTV integration site family, member 4); WNT5A (wingless-type MMTV integration site family, member 5A); WNT6 (wingless-type MMTV integration site family, member 6); WNT7B (wingless-type MMTV integration site family, member 7B); WNT8B (wingless-type MMTV integration site family, member 8B); WNT9A (wingless-type MMTV integration site family, member 9A); WNT9B (wingless-type MMTV integration site family, member 9B); WNT10A (wingless-type MMTV integration site family, member 10A); WNT10B (wingless-type MMTV integration site family, member 10B); WNT16 (wingless-type MMTV integration site family, member 16); OTX2 (orthodenticle homeobox 2); GBX2 (gastrulation brain homeobox 2); FGF8 (fibroblast growth factor 8 (androgen-induced)); RELN (reelin); DAB1 (disabled homolog 1 (Drosophila)); POU4F1 (POU class 4 homeobox 1); and NUMB (numb homolog (Drosophila).

In certain embodiments, an animal created by a method of the invention may be used to study the effects of mutations on the animal and on neurodevelopment using measures commonly used in the study of neurodevelopment.

iv. Cellular Function Models

A method of the invention may be used to create an animal or cell that may be used as a cellular function model. Such a model may be used to study the effects of an edited chromosomal sequence on a cellular function of interest. For example, a cellular function model may be used to study the effect of an edited chromosomal sequence on intracellular signaling or extracellular signaling. Or alternatively, a cellular function model may be used to study the effects of an edited chromosomal nucleic acid sequence on sensory perception.

In one embodiment, a method of the invention may be used to create an animal or cell that comprises a chromosomal edit in one or more chromosomal sequences associated with a signaling biochemical pathway. Non-limiting examples of suitable pathways and the associated nucleic acid sequences are listed in Table C.

TABLE C

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; |
| Signaling | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon Receptor | HSPB1; EP300; FASN; TGM2; RXRA; MAPK1; NQO1; |
| Signaling | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin | CD44; EP300; LRP6; DVL3; CSNK1E; GJA1; SMO; |
| Signaling | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5 |
| Tyrosine Metabolism | TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

Alternatively, a method of the invention may be used to create an animal or cell that comprises a chromosomal edit in one or more nucleic acid sequences associated with cellular function. By way of non-limiting example, a chromosomal edit may be made in a sequence associated with cognition, nociception, taste, and AB transporters, each detailed below.

### A. Cognition

In one embodiment, a method of the invention may be used to create an animal or cell in which at least one chromosomal sequence associated with cognition has been edited. Suitable chromosomal edits may include, but are not limited to, the type of edits detailed in section I(f) above.

In the foregoing embodiments, a chromosomal sequence associated with cognition may encode a cognition-related protein, or may be a control sequence. Cognition-related proteins are a diverse set of proteins that may be associated with susceptibility for developing a cognitive disorder, the presence of a cognitive disorder, the severity of a cognitive disorder or any combination thereof. Non-limiting examples of a cognitive disorder include Alzheimer's; mental retardation; Rett's syndrome; fragile X syndrome; mood disorders such as major depression disorder, unipolar disorder, mania, dysphoria, bipolar disorder, dysthymia, and cyclothymia; psychotic disorders such as schizophrenia, schizoaffective disorder, schizophreniform disorder, delusional disorder, brief psychotic disorder, substance-induced psychotic disorder, and shared psychotic disorder; personality disorders such as borderline personality disorder and dissociative identity disorder; anxiety disorders such as generalized anxiety disorder and obsessive-compulsive disorder; childhood disorders; dementia such as HIV-associated dementia (HAD) and multi-infarct dementia; autistic disorder; adjustment disorder; delirium; Tourette's disorder; attention deficit disorder; and post-traumatic stress disorder.

A cognition-related protein or control sequence may typically be selected based on an experimental association of the cognition-related sequence to a cognitive disorder. For example, the production rate or circulating concentration of a cognition-related protein may be elevated or depressed in a population having a cognitive disorder relative to a population lacking the cognitive disorder. Differences in protein levels may be assessed using proteomic or genomic analysis techniques known in the art.

Non-limiting examples of cognition-related proteins include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), ADRA1D (Alpha-1D adrenergic receptor for Alpha-1D adrenoreceptor), AHSG (Alpha-2-HS-glycoprotein), A1F1 (Allograft inflammatory factor 1), ALAS2 (Delta-aminolevulinate synthase 2), AMBP (Alpha-1-microglobulin/bikunin precursor), ANK3 (Ankryn 3), ANXA3 (Annexin A3), APCS (Amyloid P component serum), APOA1 (Apolipoprotein A1), APOA12 (Apolipoprotein A2), APOB (Apolipoprotein B), APOC1 (Apolipoprotein C1), APOE (Apolipoprotein E), APOH (Apolipoprotein H), APP (Amyloid precursor protein), ARC (Activity-regulated cytoskeleton-associated protein), ARF6 (ADP-ribosylation factor 6), ARHGAP5 (Rho GTPase activating protein 5), ASCL1 (Achaete-scute homolog 1), B2M (Beta-2 microglobulin), B4GALNT1 (Beta-1,4-N-acetyl-galactosaminyl transferase 1), BAX (BcI-2-associated X protein), BCAT (Branched chain amino-acid transaminase 1 cytosolic), BCKDHA (Branched chain keto acid dehydrogenase E1 alpha), BCKDK (Branched chain alpha-ketoacid dehydrogenase kinase), BCL2 (B-cell lymphoma 2), BCL2L1 (BCL2-like 1), BDNF (Brain-derived neurotrophic factor), BHLHE40 (Class E basic helix-loop-helix protein 40), BHLHE41 (Class E basic helix-loop-helix protein 41), BMP2 (Bone morphogenetic protein 2A), BMP3 (Bone morphogenetic protein 3), BMP5 (Bone morphogenetic protein 5), BRD1 (Bromodomain containing 1), BTC (Betacellulin), BTNL8 (Butyrophilin-like protein 8), CALB1 (Calbindin 1), CALM1 (Calmodulin 1), CAMK1 (Calcium/calmodulin-dependent protein kinase type I), CAMK4 (Calcium/calmodulin-dependent protein kinase type IV), CAMKIIB (Calcium/calmodulin-dependent protein kinase type IIB), CAMKIIG (Calcium/calmodulin-dependent protein kinase type IIG), CASP11 (Caspase-10), CASP8 (Caspase 8 apoptosis-related cysteine peptidase), CBLN1 (cerebellin 1 precursor), CCL2 (Chemokine (C-C motif) ligand 2), CCL22 (Chemokine (C-C motif) ligand 22), CCL3 (Chemokine (C-C motif) ligand 3), CCL8 (Chemokine (C-C motif) ligand 8), CCNG1 (Cyclin-G1), CCNT2 (Cyclin T2), CCR4 (C-C chemokine receptor type 4 (CD194)), CD58 (CD58), CD59 (Protectin), CD5L (CD5 antigen-like), CD93 (CD93), CDKN2AIP (CDKN2A interacting protein), CDKN2B (Cyclin-dependent kinase inhibitor 2B), CDX1 (Homeobox protein CDX-1), CEA (Carcinoembryonic antigen), CEBPA (CCAAT/enhancer-binding protein alpha), CEBPB (CCAAT/enhancer binding protein C/EBP beta), CEBPB (CCAAT/enhancer-binding protein beta), CEBPD (CCAAT/enhancer-binding protein delta), CEBPG (CCAAT/enhancer-binding protein gamma), CENPB (Centromere protein B), CGA (Glycoprotein hormone alpha chain), CGGBP1 (CGG triplet repeat-binding protein 1), CHGA (Chromogranin A), CHGB (Secretoneurin), CHN2 (Beta-chimaerin), CHRD (Chordin), CHRM1 (Cholinergic receptor muscarinic 1), CITED2 (Cbp/p300-interacting transactivator 2), CLEC4E (C-type lectin domain family 4 member E), CMTM2 (CKLF-like MARVEL transmembrane domain-containing protein 2), CNTN1 (Contactin 1), CNTNAP1 (Contactin-associated protein-like 1), CR1 (Erythrocyte complement receptor 1), CREM (cAMP-responsive element modulator), CRH (Corticotropin-releasing hormone), CRHR1 (Corticotropin releasing hormone receptor 1), CRKRS (Cell division cycle 2-related protein kinase 7), CSDA (DNA-binding protein A), CSF3 (Granulocyte colony stimulating factor 3), CSF3R (Granulocyte colony-stimulating factor 3 receptor), CSP (Chemosensory protein), CSPG4 (Chondroitin sulfate proteoglycan 4), CTCF (CCCTC-binding factor zinc finger protein), CTGF (Connective tissue growth factor), CXCL12 (Chemokine C-X-C motif ligand 12), DAD1 (Defender against cell death 1), DAXX (Death associated protein 6), DBN1 (Drebrin 1), DBP (D site of albumin promoter-albumin D-box binding protein), DDR1 (Discoidin domain receptor family member 1), DDX14 (DEAD/DEAN box helicase), DEFA3 (Defensin alpha 3 neutrophil-specific), DVL3 (Dishevelled dsh homolog 3), EDN1 (Endothelin 1), EDNRA (Endothelin receptor type A), EGF (Epidermal growth factor), EGFR (Epidermal growth factor receptor), EGR1 (Early growth response protein 1), EGR2 (Early growth response protein 2), EGR3 (Early growth response protein 3), EIF2AK2 (Eukaryotic translation initiation factor 2-alpha kinase 2), ELANE (Elastase neutrophil expressed), ELK1 (ELK1 member of ETS oncogene family), ELK3 (ELK3 ETS-domain protein (SRF accessory protein 2)), EML2 (Echinoderm microtubule associated protein like 2), EPHA4 (EPH receptor A4), ERBB2 (V-erb-b2 erythroblastic leukemia viral oncogene homolog 2), ERBB3 (Receptor tyrosine-protein kinase erbB-3), ESR2 (Estrogen receptor 2), ESR2 (Estrogen receptor 2), ETS1 (V-ets erythroblastosis virus E26 oncogene homolog 1), ETV6 (Ets variant 6), FASLG (Fas ligand TNF superfamily member 6), FCAR (Fc fragment of IgA receptor), FCER1G (Fc fragment of IgE high affinity I receptor for gamma polypeptide), FCGR2A (Fc fragment of IgG low affinity IIa receptor—CD32), FCGR3B (Fc fragment of IgG low affinity IIIb receptor—CD16b), FCGRT (Fc fragment of IgG receptor transporter alpha), FGA (Basic fibrinogen), FGF1 (Acidic fibroblast growth factor 1), FGF14 (Fibroblast growth factor 14), FGF16 (fibroblast growth factor 16), FGF18 (Fibroblast growth factor 18), FGF2 (Basic fibroblast growth factor 2), FIBP (Acidic fibroblast growth factor intracellular binding protein), FIGF (C-fos induced growth factor), FMR1 (Fragile X mental retardation 1), FOSB (FBJ murine osteosarcoma viral oncogene homolog B), FOXO1 (Forkhead box O1), FSHB (Follicle stimulating hormone beta polypeptide), FTH1 (Ferritin heavy polypeptide 1), FTL (Ferritin light polypeptide), G1P3 (Interferon alpha-inducible protein 6), G6S(N-acetylglucosamine-6-sulfatase), GABRA2 (Gamma-aminobutyric acid A receptor alpha 2), GABRA3 (Gamma-aminobutyric acid A receptor alpha 3), GABRA4 (Gamma-aminobutyric acid A receptor alpha 4), GABRB1 (Gamma-aminobutyric acid A receptor beta 1), GABRG1 (Gamma-aminobutyric acid A receptor gamma 1), GADD45A (Growth arrest and DNA-damage-inducible alpha), GCLC (Glutamate-cysteine ligase catalytic subunit), GDF15 (Growth differentiation factor 15), GDF9 (Growth differentiation factor 9), GFRA1 (GDNF family receptor alpha 1), GIT1 (G protein-coupled receptor kinase interactor 1), GNA13 (Guanine nucleotide-binding protein/G protein alpha 13), GNAQ (Guanine nucleotide binding protein/G protein q polypeptide), GPR12 (G protein-coupled receptor 12), GPR18 (G protein-coupled receptor 18), GPR22 (G protein-coupled receptor 22), GPR26 (G protein-coupled receptor 26), GPR27 (G protein-coupled receptor 27), GPR77 (G protein-coupled receptor 77), GPR85 (G protein-coupled receptor 85), GRB2 (Growth factor receptor-bound protein 2), GRLF1 (Glucocorticoid receptor DNA binding factor 1), GST (Glutathione S-transferase), GTF2B (General transcription factor IIB), GZMB (Granzyme B), HAND1 (Heart and neural crest derivatives expressed 1), HAVCR1 (Hepatitis A virus cellular receptor 1), HES1 (Hairy and enhancer of split 1), HES5 (Hairy and enhancer of split 5), HLA-DQA1 (Major histocompatibility complex class II DQ alpha), HOXA2 (Homeobox A2), HOXA4 (Homeobox A4), HP (Haptoglobin), HPGDS (Prostaglandin-D synthase), HSPA8 (Heat shock 70 kDa protein 8), HTR1A (5-hydroxytryptamine receptor 1A), HTR2A (5-hydroxytryptamine receptor 2A), HTR3A (5-hydroxytryptamine receptor 3A), ICAM1 (Intercellular adhesion molecule 1 (CD54)), IFIT2 (Interferon-induced protein with tetratricopeptide repeats 2), IFNAR2 (Interferon alpha/beta/omega receptor 2), IGF1 (Insulin-like growth factor 1), IGF2 (Insulin-like growth factor 2), IGFBP2 (Insulin-like growth factor binding protein 2, 36 kDa), IGFBP7 (Insulin-like growth factor binding protein 7), IL10 (Interleukin 10), IL10RA (Interleukin 10 receptor alpha), IL11 (Interleukin 11), IL11RA (Interleukin 11 receptor alpha), IL11RB (Interleukin 11 receptor beta), IL13 (Interleukin 13), IL15 (Interleukin 15), IL17A (Interleukin 17A), IL17RB (interleukin 17 receptor B), IL18 (Interleukin 18), IL18RAP (Interleukin 18 receptor accessory protein), IL1R2 (Interleukin 1 receptor type II), URN (Interleukin 1 receptor antagonist), IL2RA (Interleukin 2 receptor alpha), IL4R (Interleukin 4 receptor), IL6 (Interleukin 6), IL6R (Interleukin 6 receptor), IL7 (Interleukin 7), IL8 (Interleukin 8), IL8RA (Interleukin 8 receptor alpha), IL8RB (Interleukin 8 receptor beta), ILK (Integrin-linked kinase), INPP4A (Inositol polyphosphate-4-phosphatase type I, 107 kDa), INPP4B (Inositol polyphosphate-4-phosphatase type 1 beta), INS (Insulin), IRF2 (Interferon regulatory factor 2), IRF3 (Interferon regulatory factor 3), IRF9 (Interferon regulatory factor 9), IRS1 (Insulin receptor substrate 1), ITGA4 (integrin alpha 4), ITGA6 (Integrin alpha-6), ITGAE (Integrin alpha E), ITGAV (Integrin alpha-V), JAG1 (Jagged 1), JAK1 (Janus kinase 1), JDP2 (Jun dimerization protein 2), JUN (Jun oncogene), JUNB (Jun B proto-oncogene), KCNJ15 (Potassium inwardly-rectifying channel subfamily J member 15), KIF5B (Kinesin family member 5B), KLRC4 (Killer cell lectin-like receptor subfamily C member 4), KRT8 (Keratin 8), LAMP2 (Lysosomal-associated membrane protein 2), LEP (Leptin), LHB (Luteinizing hormone beta polypeptide), LRRN3 (Leucine rich repeat neuronal 3), MAL (Mal T-cell differentiation protein), MAN1A1 (Mannosidase alpha class 1A member 1), MAOB (Monoamine oxidase B), MAP3K1 (Mitogen-activated protein kinase kinase kinase 1), MAPK1 (Mitogen-activated protein kinase 1), MAPK3 (Mitogen-activated protein kinase 3), MAPRE2 (Microtubule-associated protein RP/EB family member 2), MARCKS (Myristoylated alanine-rich protein kinase C substrate), MAS1 (MAS1 oncogene), MASL1 (MAS1 oncogene-like), MBP (Myelin basic protein), MCL1 (Myeloid cell leukemia sequence 1), MDMX (MDM2-like p53-binding protein), MECP2 (Methyl CpG binding protein 2), MFGE8 (Milk fat globule-EGF factor 8 protein), MIF (Macrophage migration inhibitory factor), MMP2 (Matrix metallopeptidase 2), MOBP (Myelin-associated oligodendrocyte basic protein), MUC16 (Cancer antigen 125), MX2 (Myxovirus (influenza virus) resistance 2), MYBBP1A (MYB binding protein 1a), NBN (Nibrin), NCAM1 (Neural cell adhesion molecule 1), NCF4 (Neutrophil cytosolic factor 4 40 kDa), NCOA1 (Nuclear receptor coactivator 1), NCOA2 (Nuclear receptor coactivator 2), NEDD9 (Neural precursor cell expressed developmentally down-regulated 9), NEUR (Neuraminidase), NFATC1 (Nuclear factor of activated T-cells cytoplasmic calcineurin-dependent 1), NFE2L2 (Nuclear factor erythroid-derived 2-like 2), NFIC (Nuclear factor I/C), NFKBIA (Nuclear factor of kappa light polypeptide gene enhancer in B-cells inhibitor alpha), NGFR (Nerve growth factor receptor), NIACR2 (niacin receptor 2), NLGN3 (Neuroligin 3), NPFFR2 (neuropeptide FF receptor 2), NPY (Neuropeptide Y), NR3C2 (Nuclear receptor subfamily 3 group C member 2), NRAS (Neuroblastoma RAS viral (v-ras) oncogene homolog), NRCAM (Neuronal cell adhesion molecule), NRG1 (Neuregulin 1), NRTN (Neurturin), NRXN1 (Neurexin 1), NSMAF (Neutral sphingomyelinase activation associated factor), NTF3 (Neurotrophin 3), NTF5 (Neurotrophin 4/5), ODC1 (Ornithine decarboxylase 1), OR10A1 (Olfactory receptor 10A1), OR1A1 (Olfactory receptor family 1 subfamily A member 1), OR1N1 (Olfactory receptor family 1 subfamily N member 1), OR3A2 (Olfactory receptor family 3 subfamily A member 2), OR7A17 (Olfactory receptor family 7 subfamily A member 17), ORM1 (Orosomucoid 1), OXTR (Oxytocin receptor), P2RY13 (Purinergic receptor P2Y G-protein coupled 13), P2Y12 (Purinergic receptor P2Y G-protein coupled 12), P70S6K (P70S6 kinase), PAK1 (P21/Cdc42/Rac1-activated kinase 1), PAR1 (Prader-Willi/Angelman region-1), PBEF1 (Pre-B-cell colony enhancing factor 1), PCAF (P300/CBP-associated factor), PDE4A (cAMP-specific 3′,5′-cyclic phosphodiesterase 4A), PDE4B (Phosphodiesterase 4B cAMP-specific), PDE4B (Phosphodiesterase 4B cAMP-specific), PDE4D (Phosphodiesterase 4D cAMP-specific), PDGFA (Platelet-derived growth factor alpha polypeptide), PDGFB (Platelet-derived growth factor beta polypeptide), PDGFC (Platelet derived growth factor C), PDGFRB (Beta-type platelet-derived growth factor receptor), PDPN (Podoplanin), PENK (Enkephalin), PER1 (Period homolog 1), PLA2 (Phospholipase A2), PLAU (Plasminogen activator urokinase), PLXNC1 (Plexin C1), PMVK (Phosphomevalonate kinase), PNOC (Prepronociceptin), POLH (Polymerase (DNA directed) eta), POMC (Proopiomelanocortin (adrenocorticotropin/beta-lipotropin/alpha-melanocyte stimulating hormone/beta-melanocyte stimulating hormone/beta-endorphin)), POU2AF1 (POU domain class 2 associating factor 1), PRKAA1 (5′-AMP-activated protein kinase catalytic subunit alpha-1), PRL (Prolactin), PSCDBP (Cytohesin 1 interacting protein), PSPN (Persephin), PTAFR (Platelet-activating factor receptor), PTGS2 (Prostaglandin-endoperoxide synthase 2), PTN (Pleiotrophin), PTPN11 (Protein tyrosine phosphatase non-receptor type 11), PYY (Peptide YY), RAB11B (RAB11B member RAS oncogene family), RAB6A (RAB6A member RAS oncogene family), RAD17 (RAD17 homolog), RAF1 (RAF proto-oncogene serine/threonine-protein kinase), RANBP2 (RAN binding protein 2), RAP1A (RAP1A member of RAS oncogene family), RB1 (Retinoblastoma 1), RBL2 (Retinoblastoma-like 2 (p130)), RCVRN (Recoverin), REM2 (RAS/RAD/GEM-like GTP binding 2), RFRP (RFamide-related peptide), RPS6KA3 (Ribosomal protein S6 kinase 90 kDa polypeptide 3), RTN4 (Reticulon 4), RUNX1 (Runt-related transcription factor 1), S100A4 (S100 calcium binding protein A4), S1PR1 (Sphingosine-1-phosphate receptor 1), SCG2 (Secretogranin II), SCYE1 (Small inducible cytokine subfamily E member 1), SELENBP1 (Selenium binding protein 1), SGK (Serum/glucocorticoid regulated kinase), SKD1 (Suppressor of K+ transport growth defect 1), SLC14A1 (Solute carrier family 14 (urea transporter) member 1 (Kidd blood group)), SLC25A37 (Solute carrier family 25 member 37), SMAD2 (SMAD family member 2), SMAD5 (SMAD family member 5), SNAP23 (Synaptosomal-associated protein 23 kDa), SNCB (Synuclein beta), SNF1LK (SNF1-like kinase), SORT1 (Sortilin 1), SSB (Sjogren syndrome antigen B), STAT1 (Signal transducer and activator of transcription 1, 91 kDa), STAT5A (Signal transducer and activator of transcription 5A), STAT5B (Signal transducer and activator of transcription 5B), STX16 (Syntaxin 16), TAC1 (Tachykinin precursor 1), TBX1 (T-box 1), TEF (Thyrotrophic embryonic factor), TF (Transferrin), TGFα (Transforming growth factor alpha), TGFB1 (Transforming growth factor beta 1), TGFB2 (Transforming growth factor beta 2), TGFB3 (Transforming growth factor beta 3), TGFBR1 (Transforming growth factor beta receptor I), TGM2 (Transglutaminase 2), THPO (Thrombopoietin), TIMP1 (TIMP metallopeptidase inhibitor 1), TIMP3 (TIMP metallopeptidase inhibitor 3), TMEM129 (Transmembrane protein 129), TNFRC6 (TNFR/NGFR cysteine-rich region), TNFRSF10A (Tumor necrosis factor receptor superfamily member 10a), TNFRSF10C (Tumor necrosis factor receptor superfamily member 10c decoy without an intracellular domain), TNFRSF1A (Tumor necrosis factor receptor superfamily member 1A), TOB2 (Transducer of ERBB2 2), TOP1 (Topoisomerase (DNA) I), TOPOII (Topoisomerase 2), TRAK2 (Trafficking protein kinesin binding 2), TRH (Thyrotropin-releasing hormone), TSH (Thyroid-stimulating hormone alpha), TUBA1A (Tubulin alpha la), TXK (TXK tyrosine kinase), TYK2 (Tyrosine kinase 2), UCP1 (Uncoupling protein 1), UCP2 (Uncoupling protein 2), ULIP (Unc-33-like phosphoprotein), UTRN (Utrophin), VEGF (Vascular endothelial growth factor), VGF (VGF nerve growth factor inducible), VIP (Vasoactive intestinal peptide), VNN1 (Vanin 1), VTN (Vitronectin), WNT2 (Wingless-type MMTV integration site family member 2), XRCC6 (X-ray repair cross-complementing 6), ZEB2 (Zinc finger E-box binding homeobox 2), and ZNF461 (Zinc finger protein 461).

Exemplary cognition-related proteins include ANK3 (Ankryn 3), APP (Amyloid precursor protein), B2M (Beta-2 microglobulin), BRD1 (Bromodomain containing 1), FMR1 (Fragile X mental retardation 1), MECP2 (Methyl CpG binding protein 2), NGFR (Nerve growth factor receptor), NLGN3 (Neuroligin 3), NRXN1 (Neurexin 1) and any combination thereof.

In certain embodiments, an animal created by a method of the invention may be used to study the effects of mutations on the animal and on cognition.

### B. Nociception and Taste

Sensory-related chromosomal sequences may include but are not limited to nociception-related genes, pain-related genes, and taste-related genes. In one embodiment, a method of the invention may be used to create an animal or cell in which at least one chromosomal sequence associated with a sensory process has been edited. Suitable chromosomal edits may include, but are not limited to, the type of edits detailed in section I(f) above.

A sensory-related chromosomal sequence may be associated with nocioception or the process of receiving and responding to noxious stimuli. Non-limiting examples of nocioception-related chromosomal sequences include CALCA (calcitonin-related polypeptide alpha); FOS (FBJ murine osteosarcoma viral oncogene homolog); NPY (neuropeptide Y); TACR1 (tachykinin receptor 1); OPRM1 (opioid receptor mu 1); OPRD1 (opioid receptor delta 1); OPRK1 (opioid receptor kappa 1); TH (tyrosine hydroxylase); DRD2 (dopamine receptor D2); PTGS2 (prostaglandin-endoperoxide synthase 2 (prostaglandin G/H synthase and cyclooxygenase)); TNF (tumor necrosis factor (TNF superfamily member 2)); PDYN (prodynorphin); KNG1 (kininogen 1); CCK (cholecystokinin); NOS1 (nitric oxide synthase 1 (neuronal)); IL1B (interleukin 1 beta); SST (somatostatin); HTR3A (5-hydroxytryptamine (serotonin) receptor 3A); MAPK1 (mitogen-activated protein kinase 1); GAL (galanin prepropeptide); DYT10 (dystonia 10); TRPV1 (transient receptor potential cation channel subfamily V member 1); IL6 (interleukin 6 (interferon beta 2)); HTR2A (5-hydroxytryptamine (serotonin) receptor 2A); CNR1 (cannabinoid receptor 1 (brain)); NOS2 (nitric oxide synthase 2 inducible); PNOC (prepronociceptin); NTS (neurotensin); PTGS1 (prostaglandin-endoperoxide synthase 1 (prostaglandin G/H synthase and cyclooxygenase)); ACHE (acetylcholinesterase (Yt blood group)); NGF (nerve growth factor (beta polypeptide)); CCKBR (cholecystokinin B receptor); HTR1A (5-hydroxytryptamine (serotonin) receptor 1A); NPFF (neuropeptide FF-amide peptide precursor); CCL2 (chemokine (C-C motif) ligand 2); CAT (catalase); BDNF (brain-derived neurotrophic factor); ADORA1 (adenosine A1 receptor); NPR1 (natriuretic peptide receptor A/guanylate cyclase A (atrionatriuretic peptide receptor A)); GRP (gastrin-releasing peptide); MME (membrane metallo-endopeptidase); ABCB1 (ATP-binding cassette sub-family B (MDR/TAP) member 1); PENK (proenkephalin); TAC1 (tachykinin precursor 1); INS (insulin); NTRK1 (neurotrophic tyrosine kinase receptor type 1); SCN9A (sodium channel voltage-gated type IX alpha subunit); BCHE (butyrylcholinesterase); GALR2 (galanin receptor 2); ADCYAP1 (adenylate cyclase activating polypeptide 1 (pituitary)); HRH2 (histamine receptor H2); OXT (oxytocin prepropeptide); POMC (proopiomelanocortin); ADORA2A (adenosine A2a receptor); CPOX (coproporphyrinogen oxidase); NTSR2 (neurotensin receptor 2); SLC1A2 (solute carrier family 1 (glial high affinity glutamate transporter) member 2); OPRL1 (opiate receptor-like 1); GALR1 (galanin receptor 1); DDC (dopa decarboxylase (aromatic L-amino acid decarboxylase)); P2RX2 (purinergic receptor P2X ligand-gated ion channel 2); HMOX1 (heme oxygenase (decycling) 1); CNR2 (cannabinoid receptor 2 (macrophage)); HTR1B (5-hydroxytryptamine (serotonin) receptor 1B); HRH1 (histamine receptor H1); ADRA2A (adrenergic alpha-2A-receptor); GALR3 (galanin receptor 3); KCND1 (potassium voltage-gated channel Shal-related subfamily member 1); PRL (prolactin); IFNG (interferon gamma); GABBR1 (gamma-aminobutyric acid (GABA) B receptor 1); IL10 (interleukin 10); VWF (von Willebrand factor); GPT (glutamic-pyruvate transaminase (alanine aminotransferase)); CSF3 (colony stimulating factor 3 (granulocyte)); IL2 (interleukin 2); IFNα1 (interferon alpha 1); PROK1 (prokineticin 1); HMGCR (3-hydroxy-3-methylglutaryl-Coenzyme A reductase); JUN (jun oncogene); NPPA (natriuretic peptide precursor A); ADCY10 (adenylate cyclase 10 (soluble)); IL4 (interleukin 4); MAPK14 (mitogen-activated protein kinase 14); ADA (adenosine deaminase); TGFB1 (transforming growth factor beta 1); MAPK8 (mitogen-activated protein kinase 8); EDNRB (endothelin receptor type B); AKR1B1 (aldo-keto reductase family 1 member B1 (aldose reductase)); NOS3 (nitric oxide synthase 3 (endothelial cell)); GABRE (gamma-aminobutyric acid (GABA) A receptor epsilon); KCNJ5 (potassium inwardly-rectifying channel subfamily J member 5); EPHX2 (epoxide hydrolase 2 cytoplasmic); EDNRA (endothelin receptor type A); NTSR1 (neurotensin receptor 1 (high affinity)); IL13 (interleukin 13); EDN3 (endothelin 3); CRH (corticotropin releasing hormone); PPARA (peroxisome proliferator-activated receptor alpha); CCKAR (cholecystokinin A receptor); FAAH (fatty acid amide hydrolase); EDN1 (endothelin 1); CABIN1 (calcineurin binding protein 1); NTRK3 (neurotrophic tyrosine kinase receptor type 3); NTF3 (neurotrophin 3); PL-5283 (PL-5283 protein); APC (adenomatous polyposis coli); DBH (dopamine beta-hydroxylase (dopamine beta-monooxygenase)); SYP (synaptophysin); SLC8A1 (solute carrier family 8 (sodium/calcium exchanger) member 1); CHRNA4 (cholinergic receptor nicotinic alpha 4); TRPA1 (transient receptor potential cation channel subfamily A member 1); CYBB (cytochrome b-245 beta polypeptide); RAC1 (ras-related C3 botulinum toxin substrate 1 (rho family small GTP binding protein Rac1)); IDS (iduronate 2-sulfatase); LTF (lactotransferrin); TRPM8 (transient receptor potential cation channel subfamily M member 8); MRGPRX3 (MAS-related GPR member X3); CCR5 (chemokine (C-C motif) receptor 5); CCL5 (chemokine (C-C motif) ligand 5); MBL2 (mannose-binding lectin (protein C) 2 soluble (opsonic defect)); P2RX3 (purinergic receptor P2X ligand-gated ion channel 3); MRGPRX2 (MAS-related GPR member X2); FAM134B (family with sequence similarity 134 member B); IL8 (interleukin 8); NTRK2 (neurotrophic tyrosine kinase receptor type 2); GJA1 (gap junction protein alpha 1 43 kDa); CACNA1H (calcium channel voltage-dependent T type alpha 1H subunit); HDC (histidine decarboxylase); IFT88 (intraflagellar transport 88 homolog (Chlamydomonas)); POU4F3 (POU class 4 homeobox 3); ATOH1 (atonal homolog 1 (Drosophila)); GRM3 (glutamate receptor metabotropic 3); ADK (adenosine kinase); RIPK2 (receptor-interacting serine-threonine kinase 2); ANPEP (alanyl (membrane) aminopeptidase); DRD1 (dopamine receptor D1); NFE2L2 (nuclear factor (erythroid-derived 2)-like 2); RET (ret proto-oncogene); AHSP (alpha hemoglobin stabilizing protein); ESR2 (estrogen receptor 2 (ER beta)); HLA-A (major histocompatibility complex class I A); CHRM2 (cholinergic receptor muscarinic 2); ALAD (aminolevulinate delta-dehydratase); CXCL2 (chemokine (C-X-C motif) ligand 2); HSPG2 (heparan sulfate proteoglycan 2); F2R (coagulation factor II (thrombin) receptor); KCNIP3 (Kv channel interacting protein 3 calsenilin); GRIN1 (glutamate receptor ionotropic N-methyl D-aspartate 1); GRIK1 (glutamate receptor ionotropic kainate 1); P2RX7 (purinergic receptor P2X ligand-gated ion channel 7); CACNA1B (calcium channel voltage-dependent N type alpha 1B subunit); TACR2 (tachykinin receptor 2); NPFFR2 (neuropeptide FF receptor 2); MRGPRX1 (MAS-related GPR member X1); MRGPRX4 (MAS-related GPR member X4); PTH2 (parathyroid hormone 2); DRGX (dorsal root ganglia homeobox); CCR3 (chemokine (C-C motif) receptor 3); CYBA (cytochrome b-245 alpha polypeptide); CCL7 (chemokine (C-C motif) ligand 7); S100A6 (S100 calcium binding protein A6); CHGA (chromogranin A (parathyroid secretory protein 1)); CCL4 (chemokine (C-C motif) ligand 4); HTR5A (5-hydroxytryptamine (serotonin) receptor 5A); KCNC3 (potassium voltage-gated channel Shaw-related subfamily member 3); PNMT (phenylethanolamine N-methyltransferase); CCL8 (chemokine (C-C motif) ligand 8); LTB4R (leukotriene B4 receptor); NOXA1 (NADPH oxidase activator 1); PHOX2B (paired-like homeobox 2b); NOX1 (NADPH oxidase 1); NOX4 (NADPH oxidase 4); TAS1R3 (taste receptor type 1 member 3); NEUROG1 (neurogenin 1); NOXO1 (NADPH oxidase organizer 1); TRIM26 (tripartite motif-containing 26); OMP (olfactory marker protein); ZC3H12A (zinc finger CCCH-type containing 12A); CXCR4 (chemokine (C-X-C motif) receptor 4); PLA2G2A (phospholipase A2 group IIA (platelets synovial fluid)); PLA2G1B (phospholipase A2 group IB (pancreas)); GNRH1 (gonadotropin-releasing hormone 1 (luteinizing-releasing hormone)); TJP1 (tight junction protein 1 (zona occludens 1)); NRG1 (neuregulin 1); GRIN2B (glutamate receptor ionotropic N-methyl D-aspartate 2B); COL18A1 (collagen type XVIII alpha 1); HTR6 (5-hydroxytryptamine (serotonin) receptor 6); HTR7 (5-hydroxytryptamine (serotonin) receptor 7 (adenylate cyclase-coupled)); SLC1A3 (solute carrier family 1 (glial high affinity glutamate transporter) member 3); CACNA1D (calcium channel voltage-dependent L type alpha 1D subunit); GRM2 (glutamate receptor metabotropic 2); HNMT (histamine N-methyltransferase); ADORA2B (adenosine A2b receptor); SLC1A1 (solute carrier family 1 (neuronal/epithelial high affinity glutamate transporter system Xag) member 1); GABBR2 (gamma-aminobutyric acid (GABA) B receptor 2); PCSK2 (proprotein convertase subtilisin/kexin type 2); CD160 (CD160 molecule); TSPO (translocator protein (18 kDa)); NPSR1 (neuropeptide S receptor 1); PRO11 (proline rich lacrimal 1); NPVF (neuropeptide VF precursor); NPS (neuropeptide S); PRNP (prion protein); GRIA2 (glutamate receptor ionotropic AMPA 2); GRIA1 (glutamate receptor ionotropic AMPA 1); PRKCE (protein kinase C epsilon); ITPR1 (inositol 1 (4 (5-triphosphate receptor type 1); CBR1 (carbonyl reductase 1); ADORA3 (adenosine A3 receptor); FMR1 (fragile X mental retardation 1); ALOX5 (arachidonate 5-lipoxygenase); GRM7 (glutamate receptor metabotropic 7); PRKG1 (protein kinase cGMP-dependent type I); IL7 (interleukin 7); GRIK5 (glutamate receptor ionotropic kainate 5); HCRTR1 (hypocretin (orexin) receptor 1); CCL21 (chemokine (C-C motif) ligand 21); URN (interleukin 1 receptor antagonist); CX3CR1 (chemokine (C-X3-C motif) receptor 1); P2RX4 (purinergic receptor P2X ligand-gated ion channel 4); AVP (arginine vasopressin); PRPH (peripherin); MTOR (mechanistic target of rapamycin (serine/threonine kinase)); NFATC4 (nuclear factor of activated T-cells cytoplasmic calcineurin-dependent 4); F2RL1 (coagulation factor II (thrombin) receptor-like 1); EDN2 (endothelin 2); ACCN2 (amiloride-sensitive cation channel 2 neuronal); P2RX1 (purinergic receptor P2X ligand-gated ion channel 1); ENPEP (glutamyl aminopeptidase (aminopeptidase A)); CLDN5 (claudin 5); GFRA3 (GDNF family receptor alpha 3); PTGER1 (prostaglandin E receptor 1 (subtype EP1) 42 kDa); OCLN (occludin); P2RX5 (purinergic receptor P2X ligand-gated ion channel 5); CALB1 (calbindin 1 28 kDa); CXCL1 (chemokine (C-X-C motif) ligand 1 (melanoma growth stimulating activity alpha)); BDKRB1 (bradykinin receptor B1); TRPV4 (transient receptor potential cation channel subfamily V member 4); PRLHR (prolactin releasing hormone receptor); P2RX6 (purinergic receptor P2X ligand-gated ion channel 6); LALBA (lactalbumin alpha-); IL17A (interleukin 17A); NPFFR1 (neuropeptide FF receptor 1); ARTN (artemin); PTH2R (parathyroid hormone 2 receptor); PROK2 (prokineticin 2); PROKR2 (prokineticin receptor 2); MAS1L (MAS1 oncogene-like); PROKR1 (prokineticin receptor 1); MRGPRD (MAS-related GPR member D); MRGPRE (MAS-related GPR member E); MRGPRF (MAS-related GPR member F); and PRLH (prolactin releasing hormone).

Additionally, a sensory-related chromosomal sequence may be associated with a perception of pain. Non-limiting examples of pain-related chromosomal sequences include PTGS2 (prostaglandin-endoperoxide synthase 2 (prostaglandin G/H synthase and cyclooxygenase)); SCN9A (sodium channel voltage-gated type IX alpha subunit); TRPV1 (transient receptor potential cation channel subfamily V member 1); KNG1 (kininogen 1); IL1B (interleukin 1 beta); NTRK1 (neurotrophic tyrosine kinase receptor type 1); BDKRB1 (bradykinin receptor B1); BDKRB2 (bradykinin receptor B2); P2RX3 (purinergic receptor P2X ligand-gated ion channel 3); POMC (proopiomelanocortin); GAL (galanin prepropeptide); SCN10A (sodium channel voltage-gated type X alpha subunit); PRKCG (protein kinase C gamma); PTGS1 (prostaglandin-endoperoxide synthase 1 (prostaglandin G/H synthase and cyclooxygenase)); GRIN1 (glutamate receptor ionotropic N-methyl D-aspartate 1); NGF (nerve growth factor (beta polypeptide)); CALCA (calcitonin-related polypeptide alpha); TNF (tumor necrosis factor (TNF superfamily member 2)); IL6 (interleukin 6 (interferon beta 2)); CRP (C-reactive protein pentraxin-related); INS (insulin); OPRM1 (opioid receptor mu 1); COMT (catechol-O-methyltransferase); CNR1 (cannabinoid receptor 1 (brain)); IL10 (interleukin 10); CCK (cholecystokinin); TACR1 (tachykinin receptor 1); OPRD1 (opioid receptor delta 1); NPFFR2 (neuropeptide FF receptor 2); TGFB1 (transforming growth factor beta 1); NOS1 (nitric oxide synthase 1 (neuronal)); CRH (corticotropin releasing hormone); GALR3 (galanin receptor 3); MSD (microcephaly with spastic diplegia (Paine syndrome)); IL8 (interleukin 8); MB (myoglobin); DYT10 (dystonia 10); PRL (prolactin); MAPK1 (mitogen-activated protein kinase 1); TAC1 (tachykinin precursor 1); PDYN (prodynorphin); GCH1 (GTP cyclohydrolase 1); SOD1 (superoxide dismutase 1 soluble); SLC6A4 (solute carrier family 6 (neurotransmitter transporter serotonin) member 4); GRIN2B (glutamate receptor ionotropic N-methyl D-aspartate 2B); NPY (neuropeptide Y); OPRK1 (opioid receptor kappa 1); PENK (proenkephalin); TRPA1 (transient receptor potential cation channel subfamily A member 1); IL2 (interleukin 2); CABIN1 (calcineurin binding protein 1); NOS2 (nitric oxide synthase 2 inducible); PNOC (prepronociceptin); GRIN2A (glutamate receptor ionotropic N-methyl D-aspartate 2A); CHKA (choline kinase alpha); FOS (FBJ murine osteosarcoma viral oncogene homolog); GRIN2D (glutamate receptor ionotropic N-methyl D-aspartate 2D); CCL2 (chemokine (C-C motif) ligand 2); HTR2A (5-hydroxytryptamine (serotonin) receptor 2A); CYP19A1 (cytochrome P450 family 19 subfamily A polypeptide 1); GRIN2C (glutamate receptor ionotropic N-methyl D-aspartate 2C); PTGES (prostaglandin E synthase); HTR3A (5-hydroxytryptamine (serotonin) receptor 3A); FAAH (fatty acid amide hydrolase); NTRK2 (neurotrophic tyrosine kinase receptor type 2); ACE (angiotensin I converting enzyme (peptidyl-dipeptidase A) 1); GRM1 (glutamate receptor metabotropic 1); GDNF (glial cell derived neurotrophic factor); TLR4 (toll-like receptor 4); DRD2 (dopamine receptor D2); GRM5 (glutamate receptor metabotropic 5); VIP (vasoactive intestinal peptide); PROK1 (prokineticin 1); GALR2 (galanin receptor 2); ESR1 (estrogen receptor 1); NR3C1 (nuclear receptor subfamily 3 group C member 1 (glucocorticoid receptor)); MME (membrane metallo-endopeptidase); EDN1 (endothelin 1); NPY1R (neuropeptide Y receptor Y1); ADK (adenosine kinase); NPY2R (neuropeptide Y receptor Y2); GALR1 (galanin receptor 1); TRPC1 (transient receptor potential cation channel subfamily C member 1); TRPC5 (transient receptor potential cation channel subfamily C member 5); TRPC6 (transient receptor potential cation channel subfamily C member 6); HBS1L (HBS1-like (S. cerevisiae)); GRIN3A (glutamate receptor ionotropic N-methyl-D-aspartate 3A); GRIN3B (glutamate receptor ionotropic N-methyl-D-aspartate 3B); GPR55 (G protein-coupled receptor 55); MRGPRX3 (MAS-related GPR member X3); HSN2 (hereditary sensory neuropathy type II); AKR1B1 (aldo-keto reductase family 1 member B1 (aldose reductase)); NGFR (nerve growth factor receptor (TNFR superfamily member 16)); PRKCE (protein kinase C epsilon); TRPM8 (transient receptor potential cation channel subfamily M member 8); SST (somatostatin); IL1RN (interleukin 1 receptor antagonist); CD40LG (CD40 ligand); BCHE (butyrylcholinesterase); ACPP (acid phosphatase prostate); NPPC (natriuretic peptide precursor C); SCN11A (sodium channel voltage-gated type XI alpha subunit); KLK3 (kallikrein-related peptidase 3); PTGIR (prostaglandin I2 (prostacyclin) receptor (IP)); PPYR1 (pancreatic polypeptide receptor 1); NPY5R (neuropeptide Y receptor Y5); NPFFR1 (neuropeptide FF receptor 1); ACCN4 (amiloride-sensitive cation channel 4 pituitary); MMEL1 (membrane metallo-endopeptidase-like 1); UCN (urocortin); IFNG (interferon gamma); CYP2D6 (cytochrome P450 family 2 subfamily D polypeptide 6); CACNA1B (calcium channel voltage-dependent N type alpha 1B subunit); ACCN3 (amiloride-sensitive cation channel 3); BDNF (brain-derived neurotrophic factor); MAPK14 (mitogen-activated protein kinase 14); CNR2 (cannabinoid receptor 2 (macrophage)); MMP9 (matrix metallopeptidase 9 (gelatinase B 92 kDa gelatinase 92 kDa type IV collagenase)); IL4 (interleukin 4); ADRB2 (adrenergic beta-2-receptor surface); GFAP (glial fibrillary acidic protein); KCNIP3 (Kv channel interacting protein 3 calsenilin); URI (interleukin 1 receptor type I); ABCB1 (ATP-binding cassette sub-family B (MDR/TAP) member 1); MAPK8 (mitogen-activated protein kinase 8); MC1R (melanocortin 1 receptor (alpha melanocyte stimulating hormone receptor)); ALB (albumin); CAMK2G (calcium/calmodulin-dependent protein kinase II gamma); PLAT (plasminogen activator tissue); P2RX4 (purinergic receptor P2X ligand-gated ion channel 4); MAPK3 (mitogen-activated protein kinase 3); TNFRSF1A (tumor necrosis factor receptor superfamily member 1A); TTF2 (transcription termination factor RNA polymerase II); ITIH4 (inter-alpha (globulin) inhibitor H4 (plasma Kallikrein-sensitive glycoprotein)); CXCR4 (chemokine (C-X-C motif) receptor 4); SOD2 (superoxide dismutase 2 mitochondrial); SRC (v-src sarcoma (Schmidt-Ruppin A-2) viral oncogene homolog (avian)); PPARA (peroxisome proliferator-activated receptor alpha); CREB1 (cAMP responsive element binding protein 1); F2 (coagulation factor II (thrombin)); GAD1 (glutamate decarboxylase 1 (brain 67 kDa)); P2RX7 (purinergic receptor P2X ligand-gated ion channel 7); F3 (coagulation factor III (thromboplastin tissue factor)); MIF (macrophage migration inhibitory factor (glycosylation-inhibiting factor)); LEP (leptin); GNRH1 (gonadotropin-releasing hormone 1 (luteinizing-releasing hormone)); OPRL1 (opiate receptor-like 1); CCL3 (chemokine (C-C motif) ligand 3); UCP1 (uncoupling protein 1 (mitochondrial proton carrier)); NTS (neurotensin); SLC12A5 (solute carrier family 12 (potassium/chloride transporter) member 5); CD160 (CD160 molecule); NPFF (neuropeptide FF-amide peptide precursor); ANPEP (alanyl (membrane) aminopeptidase); VDR (vitamin D (1 (25-dihydroxyvitamin D3) receptor); JUN (jun oncogene); ADIPOQ (adiponectin C1Q and collagen domain containing); ELK1 (ELK1 member of ETS oncogene family); FGF2 (fibroblast growth factor 2 (basic)); GABBR1 (gamma-aminobutyric acid (GABA) B receptor 1); COMP (cartilage oligomeric matrix protein); SERPINE1 (serpin peptidase inhibitor clade E (nexin plasminogen activator inhibitor type 1) member 1); GRM2 (glutamate receptor metabotropic 2); GAD2 (glutamate decarboxylase 2 (pancreatic islets and brain 65 kDa)); EPO (erythropoietin); NTF3 (neurotrophin 3); IL1R2 (interleukin 1 receptor type II); ADCY1 (adenylate cyclase 1 (brain)); PEPD (peptidase D); HBEGF (heparin-binding EGF-like growth factor); GAST (gastrin); KCND1 (potassium voltage-gated channel Shal-related subfamily member 1); OXT (oxytocin prepropeptide); SLC17A5 (solute carrier family 17 (anion/sugar transporter) member 5); PL-5283 (PL-5283 protein); STN (statin); EGF (epidermal growth factor (beta-urogastrone)); CACNA1A (calcium channel voltage-dependent P/Q type alpha 1A subunit); VWF (von Willebrand factor); ANXA5 (annexin A5); MMP2 (matrix metallopeptidase 2 (gelatinase A 72 kDa gelatinase 72 kDa type IV collagenase)); HMGCR (3-hydroxy-3-methylglutaryl-Coenzyme A reductase); SPP1 (secreted phosphoprotein 1); SCN5A (sodium channel voltage-gated type V alpha subunit); GLA (galactosidase alpha); CHRNA4 (cholinergic receptor nicotinic alpha 4); PITX2 (paired-like homeodomain 2); DLG4 (discs large homolog 4 (Drosophila)); GNB3 (guanine nucleotide binding protein (G protein) beta polypeptide 3); ADORA1 (adenosine A1 receptor); MYH7 (myosin heavy chain 7 cardiac muscle beta); TXN (thioredoxin); CP (ceruloplasmin (ferroxidase)); CSF3 (colony stimulating factor 3 (granulocyte)); SLC1A1 (solute carrier family 1 (neuronal/epithelial high affinity glutamate transporter system Xag) member 1); IAPP (islet amyloid polypeptide); GUK1 (guanylate kinase 1); NPPA (natriuretic peptide precursor A); ADCYAP1 (adenylate cyclase activating polypeptide 1 (pituitary)); XDH (xanthine dehydrogenase); SRD5A1 (steroid-5-alpha-reductase alpha polypeptide 1 (3-oxo-5 alpha-steroid delta 4-dehydrogenase alpha 1)); IDO1 (indoleamine 2 (3-dioxygenase 1); REN (renin); CX3CL1 (chemokine (C-X3-C motif) ligand 1); NEK3 (NIMA (never in mitosis gene a)-related kinase 3); KIAA0101 (KIAA0101); ARTN (artemin); SLC17A6 (solute carrier family 17 (sodium-dependent inorganic phosphate cotransporter) member 6); GPR172B (G protein-coupled receptor 172B); BCL2 (B-cell CLL/lymphoma 2); CREBBP (CREB binding protein); NCAM1 (neural cell adhesion molecule 1); EPOR (erythropoietin receptor); ATP2A2 (ATPase Ca++ transporting cardiac muscle slow twitch 2); HTR7 (5-hydroxytryptamine (serotonin) receptor 7 (adenylate cyclase-coupled)); MYH11 (myosin heavy chain 11 smooth muscle); AGTR2 (angiotensin II receptor type 2); ENO2 (enolase 2 (gamma neuronal)); VIM (vimentin); MAP2K3 (mitogen-activated protein kinase kinase 3); ADAM17 (ADAM metallopeptidase domain 17); IL6ST (interleukin 6 signal transducer (gp130 oncostatin M receptor)); PSMA2 (proteasome (prosome macropain) subunit alpha type 2); MAP2K6 (mitogen-activated protein kinase kinase 6); S100A9 (S100 calcium binding protein A9); S100A8 (S100 calcium binding protein A8); CCL21 (chemokine (C-C motif) ligand 21); EPHA4 (EPH receptor A4); ADCYAP1R1 (adenylate cyclase activating polypeptide 1 (pituitary) receptor type I); CGB (chorionic gonadotropin beta polypeptide); IBSP (integrin-binding sialoprotein); SORT1 (sortilin 1); CNTF (ciliary neurotrophic factor); DAO (D-amino-acid oxidase); NRTN (neurturin); HCRT (hypocretin (orexin) neuropeptide precursor); MAP1B (microtubule-associated protein 1B); ADAMTS13 (ADAM metallopeptidase with thrombospondin type 1 motif 13); ABP1 (amiloride binding protein 1 (amine oxidase (copper-containing))); SLC17A7 (solute carrier family 17 (sodium-dependent inorganic phosphate cotransporter) member 7); CADM1 (cell adhesion molecule 1); AIF1 (allograft inflammatory factor 1); ADCY10 (adenylate cyclase 10 (soluble)); TRIM26 (tripartite motif-containing 26); GGT2 (gamma-glutamyltransferase 2); ILIA (interleukin 1 alpha); CIS (complement component 1 s subcomponent); MPO (myeloperoxidase); NPPB (natriuretic peptide precursor B); F2RL1 (coagulation factor II (thrombin) receptor-like 1); TNNI3 (troponin I type 3 (cardiac)); SELP (selectin P (granule membrane protein 140 kDa antigen CD62)); TNFRSF11B (tumor necrosis factor receptor superfamily member 11b); FABP3 (fatty acid binding protein 3 muscle and heart (mammary-derived growth inhibitor)); ADRA2A (adrenergic alpha-2A-receptor); HTR1A (5-hydroxytryptamine (serotonin) receptor 1A); CASP3 (caspase 3 apoptosis-related cysteine peptidase); CPOX (coproporphyrinogen oxidase); SCN7A (sodium channel voltage-gated type VII alpha); PPARG (peroxisome proliferator-activated receptor gamma); MYL3 (myosin light chain 3 alkali; ventricular skeletal slow); CRHR1 (corticotropin releasing hormone receptor 1); ICAM1 (intercellular adhesion molecule 1); MAPK10 (mitogen-activated protein kinase 10); CAMK2A (calcium/calmodulin-dependent protein kinase II alpha); EDNRB (endothelin receptor type B); CSF2 (colony stimulating factor 2 (granulocyte-macrophage)); SCN4A (sodium channel voltage-gated type IV alpha subunit); EPRS (glutamyl-prolyl-tRNA synthetase); HBB (hemoglobin beta); IL5 (interleukin 5 (colony-stimulating factor eosinophil)); EDNRA (endothelin receptor type A); MEFV (Mediterranean fever); PAPPA (pregnancy-associated plasma protein A pappalysin 1); PTGER4 (prostaglandin E receptor 4 (subtype EP4)); PIK3C2A (phosphoinositide-3-kinase class 2 alpha polypeptide); BGLAP (bone gamma-carboxyglutamate (gla) protein); POR (P450 (cytochrome) oxidoreductase); NOS3 (nitric oxide synthase 3 (endothelial cell)); PRKACA (protein kinase cAMP-dependent catalytic alpha); TP53 (tumor protein p53); RPS6KB1 (ribosomal protein S6 kinase 70 kDa polypeptide 1); PRKAR1A (protein kinase cAMP-dependent regulatory type 1 alpha (tissue specific extinguisher 1)); IGF1 (insulin-like growth factor 1 (somatomedin C)); GRIA2 (glutamate receptor ionotropic AMPA 2); GRIA1 (glutamate receptor ionotropic AMPA 1); IL13 (interleukin 13); HSP90AA1 (heat shock protein 90 kDa alpha (cytosolic) class A member 1); PIK3CG (phosphoinositide-3-kinase catalytic gamma polypeptide); IL12B (interleukin 12B (natural killer cell stimulatory factor 2 cytotoxic lymphocyte maturation factor 2 p40)); CYP3A4 (cytochrome P450 family 3 subfamily A polypeptide 4); PRKACB (protein kinase cAMP-dependent catalytic beta); PRKAR2A (protein kinase cAMP-dependent regulatory type II alpha); GRM8 (glutamate receptor metabotropic 8); CAMK2D (calcium/calmodulin-dependent protein kinase II delta); GRM7 (glutamate receptor metabotropic 7); GH1 (growth hormone 1); TNNT2 (troponin T type 2 (cardiac)); MAOA (monoamine oxidase A); CAMK2B (calcium/calmodulin-dependent protein kinase II beta); SERPINC1 (serpin peptidase inhibitor clade C (antithrombin) member 1); SLC12A2 (solute carrier family 12 (sodium/potassium/chloride transporters) member 2); COL2A1 (collagen type II alpha 1); PRKAR1B (protein kinase cAMP-dependent regulatory type 1 beta); CX3CR1 (chemokine (C-X3-C motif) receptor 1); PRKACG (protein kinase cAMP-dependent catalytic gamma); SLC6A2 (solute carrier family 6 (neurotransmitter transporter noradrenalin) member 2); MTOR (mechanistic target of rapamycin (serine/threonine kinase)); DLG2 (discs large homolog 2 (Drosophila)); MGLL (monoglyceride lipase); ATF3 (activating transcription factor 3); ALPP (alkaline phosphatase placental (Regan isozyme)); COL9A2 (collagen type IX alpha 2); HBG2 (hemoglobin gamma G); MRGPRX1 (MAS-related GPR member X1); FGFR1 (fibroblast growth factor receptor 1); NFKB1 (nuclear factor of kappa light polypeptide gene enhancer in B-cells 1); EIF4E (eukaryotic translation initiation factor 4E); PRKCA (protein kinase C alpha); EGFR (epidermal growth factor receptor (erythroblastic leukemia viral (v-erb-b) oncogene homolog avian)); PIK3R1 (phosphoinositide-3-kinase regulatory subunit 1 (alpha)); PTPN6 (protein tyrosine phosphatase non-receptor type 6); PLCG2 (phospholipase C gamma 2 (phosphatidylinositol-specific)); PRKCQ (protein kinase C theta); PLG (plasminogen); GRIA3 (glutamate receptor ionotrophic AMPA 3); IL6R (interleukin 6 receptor); HIF1A (hypoxia inducible factor 1 alpha subunit (basic helix-loop-helix transcription factor)); ALPL (alkaline phosphatase liver/bone/kidney); ADCY6 (adenylate cyclase 6); PRKCZ (protein kinase C zeta); GRM3 (glutamate receptor metabotropic 3); IL2RA (interleukin 2 receptor alpha); PIK3CD (phosphoinositide-3-kinase catalytic delta polypeptide); SNCA (synuclein alpha (non A4 component of amyloid precursor)); CYP1A1 (cytochrome P450 family 1 subfamily A polypeptide 1); PLCG1 (phospholipase C gamma 1); DBH (dopamine beta-hydroxylase (dopamine beta-monooxygenase)); GRIK1 (glutamate receptor ionotropic kainate 1); PRKCH (protein kinase C eta); PRKCD (protein kinase C delta); CAT (catalase); ITPR1 (inositol 1 (4 (5-triphosphate receptor type 1); PLCB3 (phospholipase C beta 3 (phosphatidylinositol-specific)); PLCB2 (phospholipase C beta 2); PIK3CB (phosphoinositide-3-kinase catalytic beta polypeptide); PLA2G2A (phospholipase A2 group IIA (platelets synovial fluid)); PIK3CA (phosphoinositide-3-kinase catalytic alpha polypeptide); DRD3 (dopamine receptor D3); DMD (dystrophin); MAPK7 (mitogen-activated protein kinase 7); PIK3C3 (phosphoinositide-3-kinase class 3); LPL (lipoprotein lipase); ADCY8 (adenylate cyclase 8 (brain)); HSPG2 (heparan sulfate proteoglycan 2); CCL5 (chemokine (C-C motif) ligand 5); ALOX5 (arachidonate 5-lipoxygenase); PRKCI (protein kinase C iota); PRKAR2B (protein kinase cAMP-dependent regulatory type II beta); GLRA1 (glycine receptor alpha 1); MMP12 (matrix metallopeptidase 12 (macrophage elastase)); CHAT (choline acetyltransferase); LRP5 (low density lipoprotein receptor-related protein 5); TIMP1 (TIMP metallopeptidase inhibitor 1); PLCB1 (phospholipase C beta 1 (phosphoinositide-specific)); F2R (coagulation factor II (thrombin) receptor); EIF2S1 (eukaryotic translation initiation factor 2 subunit 1 alpha 35 kDa); SELL (selectin L); THBS2 (thrombospondin 2); ADRA2C (adrenergic alpha-2C-receptor); HTR2B (5-hydroxytryptamine (serotonin) receptor 2B); TF (transferrin); CST3 (cystatin C); PIK3C2B (phosphoinositide-3-kinase class 2 beta polypeptide); PLCD1 (phospholipase C delta 1); PLCB4 (phospholipase C beta 4); NR112 (nuclear receptor subfamily 1 group I member 2); PIK3R2 (phosphoinositide-3-kinase regulatory subunit 2 (beta)); PYGM (phosphorylase glycogen muscle); KCNQ3 (potassium voltage-gated channel KQT-like subfamily member 3); PECAM1 (platelet/endothelial cell adhesion molecule); CCL4 (chemokine (C-C motif) ligand 4); TACR3 (tachykinin receptor 3); GRM4 (glutamate receptor metabotropic 4); 9-Sep (septin 9); LBP (lipopolysaccharide binding protein); CAMK1 (calcium/calmodulin-dependent protein kinase I); SCN1A (sodium channel voltage-gated type 1 alpha subunit); OSM (oncostatin M); SQSTM1 (sequestosome 1); AVP (arginine vasopressin); PRPH (peripherin); GLRA3 (glycine receptor alpha 3); PIK3R3 (phosphoinositide-3-kinase regulatory subunit 3 (gamma)); PTGER3 (prostaglandin E receptor 3 (subtype EP3)); SPTLC1 (serine palmitoyltransferase long chain base subunit 1); PIK3C2G (phosphoinositide-3-kinase class 2 gamma polypeptide); PTH (parathyroid hormone); TJP1 (tight junction protein 1 (zona occludens 1)); SCN2B (sodium channel voltage-gated type II beta); EIF2AK2 (eukaryotic translation initiation factor 2-alpha kinase 2); CACNA2D2 (calcium channel voltage-dependent alpha 2/delta subunit 2); ADCY5 (adenylate cyclase 5); PRKCB (protein kinase C beta); TAT (tyrosine aminotransferase); CLDN5 (claudin 5); HYAL1 (hyaluronoglucosaminidase 1); PLCD3 (phospholipase C delta 3); PTGER1 (prostaglandin E receptor 1 (subtype EP1) 42 kDa); KRT7 (keratin 7); PPIG (peptidylprolyl isomerase G (cyclophilin G)); OCLN (occludin); CACNA2D1 (calcium channel voltage-dependent alpha 2/delta subunit 1); CXCL1 (chemokine (C-X-C motif) ligand 1 (melanoma growth stimulating activity alpha)); SLC6A1 (solute carrier family 6 (neurotransmitter transporter GABA) member 1); SERPINA6 (serpin peptidase inhibitor clade A (alpha-1 antiproteinase antitrypsin) member 6); TRPV4 (transient receptor potential cation channel subfamily V member 4); NNT (nicotinamide nucleotide transhydrogenase); GRM6 (glutamate receptor metabotropic 6); DPP3 (dipeptidyl-peptidase 3); SLC18A3 (solute carrier family 18 (vesicular acetylcholine) member 3); GPT (glutamic-pyruvate transaminase (alanine aminotransferase)); TFIP11 (tuftelin interacting protein 11); KCNK2 (potassium channel subfamily K member 2); CYB5A (cytochrome b5 type A (microsomal)); PLCZ1 (phospholipase C zeta 1); ANK3 (ankyrin 3 node of Ranvier (ankyrin G)); BLVRB (biliverdin reductase B (flavin reductase (NADPH))); FGF23 (fibroblast growth factor 23); CAMK1G (calcium/calmodulin-dependent protein kinase IG); TRPV2 (transient receptor potential cation channel subfamily V member 2); PIK3R5 (phosphoinositide-3-kinase regulatory subunit 5); GRINA (glutamate receptor ionotropic N-methyl D-aspartate-associated protein 1 (glutamate binding)); PROK2 (prokineticin 2); ENAM (enamelin); NPBWR1 (neuropeptides B/W receptor 1); LXN (latexin); MRGPRX2 (MAS-related GPR member X2); AMBN (ameloblastin (enamel matrix protein)); UCN2 (urocortin 2); TUFT1 (tuftelin 1); FAM134B (family with sequence similarity 134 member B); TAC4 (tachykinin 4 (hemokinin)); NPB (neuropeptide B); PDGFRB (platelet-derived growth factor receptor beta polypeptide); ITGB2 (integrin beta 2 (complement component 3 receptor 3 and 4 subunit)); FGFR2 (fibroblast growth factor receptor 2); TSC1 (tuberous sclerosis 1); RUNX1 (runt-related transcription factor 1); PTPRC (protein tyrosine phosphatase receptor type C); FYN (FYN oncogene related to SRC FGR YES); APP (amyloid beta (A4) precursor protein); PGR (progesterone receptor); ERBB2 (v-erb-b2 erythroblastic leukemia viral oncogene homolog 2 neuro/glioblastoma derived oncogene homolog (avian)); ERBB3 (v-erb-b2 erythroblastic leukemia viral oncogene homolog 3 (avian)); CSTB (cystatin B (stefin B)); CASP8 (caspase 8 apoptosis-related cysteine peptidase); ADA (adenosine deaminase); WT1 (Wilms tumor 1); CD44 (CD44 molecule (Indian blood group)); NFKBIA (nuclear factor of kappa light polypeptide gene enhancer in B-cells inhibitor alpha); RB1 (retinoblastoma 1); S100B (S100 calcium binding protein B); MYL2 (myosin light chain 2 regulatory cardiac slow); PSEN1 (presenilin 1); EGR1 (early growth response 1); GJA1 (gap junction protein alpha 1 43 kDa); SLC6A3 (solute carrier family 6 (neurotransmitter transporter dopamine) member 3); JAK2 (Janus kinase 2); RYR1 (ryanodine receptor 1 (skeletal)); CCKBR (cholecystokinin B receptor); RELA (v-rel reticuloendotheliosis viral oncogene homolog A (avian)); RET (ret proto-oncogene); ANXA2 (annexin A2); CCR5 (chemokine (C-C motif) receptor 5); TGFBR1 (transforming growth factor beta receptor 1); PARK2 (Parkinson disease (autosomal recessive juvenile) 2 parkin); ITGA6 (integrin alpha 6); DPYD (dihydropyrimidine dehydrogenase); TH (tyrosine hydroxylase); GNAS (GNAS complex locus); TNFRSF1B (tumor necrosis factor receptor superfamily member 1B); COL1A1 (collagen type 1 alpha 1); HMOX1 (heme oxygenase (decycling) 1); LDHA (lactate dehydrogenase A); MBP (myelin basic protein); SERPINA1 (serpin peptidase inhibitor clade A (alpha-1 antiproteinase antitrypsin) member 1); SCNN1A (sodium channel nonvoltage-gated 1 alpha); ACTN2 (actinin alpha 2); ACHE (acetylcholinesterase (Yt blood group)); TTN (titin); CCNH (cyclin H); SLC1A2 (solute carrier family 1 (glial high affinity glutamate transporter) member 2); ESR2 (estrogen receptor 2 (ER beta)); HTR4 (5-hydroxytryptamine (serotonin) receptor 4); KCNH2 (potassium voltage-gated channel subfamily H (eag-related) member 2); ADRBK1 (adrenergic beta receptor kinase 1); IRS1 (insulin receptor substrate 1); C3 (complement component 3); LTA4H (leukotriene A4 hydrolase); GSR (glutathione reductase); NF2 (neurofibromin 2 (merlin)); ATF2 (activating transcription factor 2); IGFBP3 (insulin-like growth factor binding protein 3); BMP4 (bone morphogenetic protein 4); CDK5 (cyclin-dependent kinase 5); CDC25C (cell division cycle 25 homolog C (S. pombe)); CD36 (CD36 molecule (thrombospondin receptor)); TPM1 (tropomyosin 1 (alpha)); CD40 (CD40 molecule TNF receptor superfamily member 5); CYP1A2 (cytochrome P450 family 1 subfamily A polypeptide 2); FN1 (fibronectin 1); PKM2 (pyruvate kinase muscle); G6PD (glucose-6-phosphate dehydrogenase); CGA (glycoprotein hormones alpha polypeptide); HSF1 (heat shock transcription factor 1); CD3E (CD3e molecule epsilon (CD3-TCR complex)); CYP3A5 (cytochrome P450 family 3 subfamily A polypeptide 5); CYP2C9 (cytochrome P450 family 2 subfamily C polypeptide 9); ADRA1A (adrenergic alpha-1A-receptor); CD14 (CD14 molecule); IL4R (interleukin 4 receptor); ITPR3 (inositol 1 (4 (5-triphosphate receptor type 3); IL15 (interleukin 15); MECP2 (methyl CpG binding protein 2 (Rett syndrome)); ANXA1 (annexin A1); PRKAG1 (protein kinase AMP-activated gamma 1 non-catalytic subunit); DCN (decorin); MYB (v-myb myeloblastosis viral oncogene homolog (avian)); AVPR1A (arginine vasopressin receptor 1A); HLA-DQB1 (major histocompatibility complex class II DQ beta 1); NEFL (neurofilament light polypeptide); SCNN1B (sodium channel nonvoltage-gated 1 beta); CACNA1H (calcium channel voltage-dependent T type alpha 1H subunit); IFNAR1 (interferon (alpha beta and omega) receptor 1); PDE4D (phosphodiesterase 4D cAMP-specific (phosphodiesterase E3 dunce homolog Drosophila)); HDAC9 (histone deacetylase 9); ABCC1 (ATP-binding cassette sub-family C(CFTR/MRP) member 1); PRDX5 (peroxiredoxin 5); EPHX2 (epoxide hydrolase 2 cytoplasmic); VCAM1 (vascular cell adhesion molecule 1); PRKAG2 (protein kinase AMP-activated gamma 2 non-catalytic subunit); ADCY2 (adenylate cyclase 2 (brain)); HTR1B (5-hydroxytryptamine (serotonin) receptor 1B); ADCY9 (adenylate cyclase 9); HLA-A (major histocompatibility complex class I A); SLC1A3 (solute carrier family 1 (glial high affinity glutamate transporter) member 3); HLA-B (major histocompatibility complex class I B); ITGA2 (integrin alpha 2 (CD49B alpha 2 subunit of VLA-2 receptor)); GABRA2 (gamma-aminobutyric acid (GABA) A receptor alpha 2); IL2RB (interleukin 2 receptor beta); GLRB (glycine receptor beta); SOCS3 (suppressor of cytokine signaling 3); CSNK2B (casein kinase 2 beta polypeptide); KCNK3 (potassium channel subfamily K member 3); KCNQ2 (potassium voltage-gated channel KQT-like subfamily member 2); DPYSL2 (dihydropyrimidinase-like 2); CYP2J2 (cytochrome P450 family 2 subfamily J polypeptide 2); DRD4 (dopamine receptor D4); PRKG1 (protein kinase cGMP-dependent type I); TNFSF11 (tumor necrosis factor (ligand) superfamily member 11); IFNAR2 (interferon (alpha beta and omega) receptor 2); EIF4EBP1 (eukaryotic translation initiation factor 4E binding protein 1); EIF4G1 (eukaryotic translation initiation factor 4 gamma 1); EIF4G3 (eukaryotic translation initiation factor 4 gamma 3); SCNN1G (sodium channel nonvoltage-gated 1 gamma); SERPING1 (serpin peptidase inhibitor clade G (C1 inhibitor) member 1); PABPN1 (poly(A) binding protein nuclear 1); CAST (calpastatin); CTSC (cathepsin C); CTGF (connective tissue growth factor); CHRNB2 (cholinergic receptor nicotinic beta 2 (neuronal)); ADCY3 (adenylate cyclase 3); ADCY7 (adenylate cyclase 7); ADRA1D (adrenergic alpha-1D-receptor); CHRM2 (cholinergic receptor muscarinic 2); DHFR (dihydrofolate reductase); MC2R (melanocortin 2 receptor (adrenocorticotropic hormone)); THBD (thrombomodulin); IL7 (interleukin 7); IL18 (interleukin 18 (interferon-gamma-inducing factor)); SIRT1 (sirtuin (silent mating type information regulation 2 homolog) 1 (S. cerevisiae)); GRIA4 (glutamate receptor ionotrophic AMPA4); CSNK1E (casein kinase 1 epsilon); CPE (carboxypeptidase E); PRSS1 (protease serine 1 (trypsin 1)); GOT2 (glutamic-oxaloacetic transaminase 2 mitochondrial (aspartate aminotransferase 2)); GABRB1 (gamma-aminobutyric acid (GABA) A receptor beta 1); ALOX12 (arachidonate 12-lipoxygenase); CCL11 (chemokine (C-C motif) ligand 11); HLA-DRB1 (major histocompatibility complex class II DR beta 1); RBL2 (retinoblastoma-like 2 (p130)); AGER (advanced glycosylation end product-specific receptor); LAMP1 (lysosomal-associated membrane protein 1); MAPKAPK2 (mitogen-activated protein kinase-activated protein kinase 2); LTA (lymphotoxin alpha (TNF superfamily member 1)); CYP4A11 (cytochrome P450 family 4 subfamily A polypeptide 11); MAOB (monoamine oxidase B); TPH1 (tryptophan hydroxylase 1); SPARC (secreted protein acidic cysteine-rich (osteonectin)); PIK3R4 (phosphoinositide-3-kinase regulatory subunit 4); CYP17A1 (cytochrome P450 family 17 subfamily A polypeptide 1); CD63 (CD63 molecule); CLCN1 (chloride channel 1 skeletal muscle); NFE2L2 (nuclear factor (erythroid-derived 2)-like 2); TNFRSF11A (tumor necrosis factor receptor superfamily member 11a NFKB activator); CRHR2 (corticotropin releasing hormone receptor 2); COPE (coatomer protein complex subunit epsilon); CYP4F2 (cytochrome P450 family 4 subfamily F polypeptide 2); APOB (apolipoprotein B (including Ag(x) antigen)); GFRA1 (GDNF family receptor alpha 1); HMBS (hydroxymethylbilane synthase); F5 (coagulation factor V (proaccelerin labile factor)); TPO (thyroid peroxidase); AMPH (amphiphysin); PTGER2 (prostaglandin E receptor 2 (subtype EP2) 53 kDa); PKLR (pyruvate kinase liver and RBC); SMPD1 (sphingomyelin phosphodiesterase 1 acid lysosomal); PLA2G4A (phospholipase A2 group IVA (cytosolic calcium-dependent)); JUNB (jun B proto-oncogene); GSN (gelsolin); PLCE1 (phospholipase C epsilon 1); PSMB8 (proteasome (prosome macropain) subunit beta type 8 (large multifunctional peptidase 7)); CYCS (cytochrome c somatic); KCNK1 (potassium channel subfamily K member 1); PGF (placental growth factor); IL10RA (interleukin 10 receptor alpha); CHRM1 (cholinergic receptor muscarinic 1); IL12RB1 (interleukin 12 receptor beta 1); CHGA (chromogranin A (parathyroid secretory protein 1)); GABRE (gamma-aminobutyric acid (GABA) A receptor epsilon); GJA4 (gap junction protein alpha 4 37 kDa); ALAD (aminolevulinate delta-dehydratase); GLRA2 (glycine receptor alpha 2); ITPR2 (inositol 1 (4 (5-triphosphate receptor type 2); MPZ (myelin protein zero); AQP1 (aquaporin 1 (Colton blood group)); MYBPC3 (myosin binding protein C cardiac); CPT2 (carnitine palmitoyltransferase 2); STAR (steroidogenic acute regulatory protein); GLB1 (galactosidase beta 1); SCN8A (sodium channel voltage gated type VIII alpha subunit); LGALS1 (lectin galactoside-binding soluble 1); PCSK1 (proprotein convertase subtilisin/kexin type 1); IKBKAP (inhibitor of kappa light polypeptide gene enhancer in B-cells kinase complex-associated protein); REST (RE1-silencing transcription factor); OXTR (oxytocin receptor); UGT2B7 (UDP glucuronosyltransferase 2 family polypeptide B7); LTF (lactotransferrin); TYRP1 (tyrosinase-related protein 1); RBL1 (retinoblastoma-like 1 (p107)); TCAP (titin-cap (telethonin)); KCNJ1 (potassium inwardly-rectifying channel subfamily J member 1); KCNN3 (potassium intermediate/small conductance calcium-activated channel subfamily N member 3); PSMC1 (proteasome (prosome macropain) 26S subunit ATPase 1); RELN (reelin); MYH14 (myosin heavy chain 14 non-muscle); ADCY4 (adenylate cyclase 4); MMP10 (matrix metallopeptidase 10 (stromelysin 2)); FXN (frataxin); ATF4 (activating transcription factor 4 (tax-responsive enhancer element B67)); NOG (noggin); PPDX (protoporphyrinogen oxidase); TNNC1 (troponin C type 1 (slow)); HRH2 (histamine receptor H2); PLA2G4C (phospholipase A2 group IVC (cytosolic calcium-independent)); NR3C2 (nuclear receptor subfamily 3 group C member 2); AMPD1 (adenosine monophosphate deaminase 1); FKBP4 (FK506 binding protein 4 59 kDa); MBD2 (methyl-CpG binding domain protein 2); NRG1 (neuregulin 1); MBL2 (mannose-binding lectin (protein C) 2 soluble (opsonic defect)); AGA (aspartylglucosaminidase); SP1 (Sp1 transcription factor); SCN3A (sodium channel voltage-gated type III alpha subunit); FABP2 (fatty acid binding protein 2 intestinal); PABPC1 (poly(A) binding protein cytoplasmic 1); ACCN2 (amiloride-sensitive cation channel 2 neuronal); ACTC1 (actin alpha cardiac muscle 1); ACP5 (acid phosphatase 5 tartrate resistant); EIF4B (eukaryotic translation initiation factor 4B); EIF4EBP2 (eukaryotic translation initiation factor 4E binding protein 2); EIF4A1 (eukaryotic translation initiation factor 4A1); CAMK4 (calcium/calmodulin-dependent protein kinase IV); CACNB3 (calcium channel voltage-dependent beta 3 subunit); CAV3 (caveolin 3); CA6 (carbonic anhydrase VI); ALOX12B (arachidonate 12-lipoxygenase 12R type); CCL17 (chemokine (C-C motif) ligand 17); CCL22 (chemokine (C-C motif) ligand 22); MMP20 (matrix metallopeptidase 20); GAP43 (growth associated protein 43); ALOX5AP (arachidonate 5-lipoxygenase-activating protein); ANTXR2 (anthrax toxin receptor 2); HGD (homogentisate 1 (2-dioxygenase); SELE (selectin E); MYLK2 (myosin light chain kinase 2); VEGFA (vascular endothelial growth factor A); PRX (periaxin); IL10RB (interleukin 10 receptor beta); HAS1 (hyaluronan synthase 1); GTF2IRD1 (GTF2I repeat domain containing 1); IL16 (interleukin 16 (lymphocyte chemoattractant factor)); GRIP1 (glutamate receptor interacting protein 1); PHKA1 (phosphorylase kinase alpha 1 (muscle)); FOXP3 (forkhead box P3); SFTPC (surfactant protein C); PDIA3 (protein disulfide isomerase family A member 3); SRM (spermidine synthase); MARCKS (myristoylated alanine-rich protein kinase C substrate); RAPGEF3 (Rap guanine nucleotide exchange factor (GEF) 3); RAGE (renal tumor antigen); MRC1 (mannose receptor C type 1); SPINK1 (serine peptidase inhibitor Kazal type 1); CYP4F3 (cytochrome P450 family 4 subfamily F polypeptide 3); LPIN1 (lipin 1); TREX1 (three prime repair exonuclease 1); CYSLTR2 (cysteinyl leukotriene receptor 2); PTX3 (pentraxin 3 long); PTGES2 (prostaglandin E synthase 2); ASAH1 (N-acylsphingosine amidohydrolase (acid ceramidase) 1); H2AFZ (H2A histone family member Z); HFE (hemochromatosis); PYGB (phosphorylase glycogen; brain); NR2F6 (nuclear receptor subfamily 2 group F member 6); CYP3A7 (cytochrome P450 family 3 subfamily A polypeptide 7); RAB6A (RAB6A member RAS oncogene family); F2RL3 (coagulation factor II (thrombin) receptor-like 3); RGS4 (regulator of G-protein signaling 4); SCNN1D (sodium channel nonvoltage-gated 1 delta); SCN1B (sodium channel voltage-gated type 1 beta); SCN2A (sodium channel voltage-gated type II alpha subunit); CALCRL (calcitonin receptor-like); CALB1 (calbindin 1 28 kDa); CACNG2 (calcium channel voltage-dependent gamma subunit 2); TACR2 (tachykinin receptor 2); GPC3 (glypican 3); GALNT3 (UDP-N-acetyl-alpha-D-galactosamine:polypeptide N-acetylgalactosaminyltransferase 3 (GalNAc-T3)); CXCL10 (chemokine (C-X-C motif) ligand 10); ANKH (ankylosis progressive homolog (mouse)); PRKD1 (protein kinase D1); KCNN4 (potassium intermediate/small conductance calcium-activated channel subfamily N member 4); TGM1 (transglutaminase 1 (K polypeptide epidermal type I protein-glutamine-gamma-glutamyltransferase)); SLC26A2 (solute carrier family 26 (sulfate transporter) member 2); MTNR1A (melatonin receptor 1A); MIPEP (mitochondrial intermediate peptidase); SI (sucrase-isomaltase (alpha-glucosidase)); RHAG (Rh-associated glycoprotein); SLC12A3 (solute carrier family 12 (sodium/chloride transporters) member 3); RNASE1 (ribonuclease RNase A family 1 (pancreatic)); ELANE (elastase neutrophil expressed); GPC6 (glypican 6); ENPP2 (ectonucleotide pyrophosphatase/phosphodiesterase 2); SCN3B (sodium channel voltage-gated type III beta); CALB2 (calbindin 2); CTSA (cathepsin A); EIF2AK1 (eukaryotic translation initiation factor 2-alpha kinase 1); TMSB4X (thymosin beta 4 X-linked); LPO (lactoperoxidase); NDN (necdin homolog (mouse)); PICK1 (protein interacting with PRKCA 1); PLCD4 (phospholipase C delta 4); CLDN3 (claudin 3); HCN1 (hyperpolarization activated cyclic nucleotide-gated potassium channel 1); MATN3 (matrilin 3); COL9A3 (collagen type IX alpha 3); BTG1 (B-cell translocation gene 1 anti-proliferative); LCN1 (lipocalin 1 (tear prealbumin)); FDX1 (ferredoxin 1); UTRN (utrophin); FMOD (fibromodulin); PDE4A (phosphodiesterase 4A cAMP-specific (phosphodiesterase E2 dunce homolog Drosophila)); RRBP1 (ribosome binding protein 1 homolog 180 kDa (dog)); MLYCD (malonyl-CoA decarboxylase); ANXA3 (annexin A3); PRKD3 (protein kinase D3); GHRL (ghrelin/obestatin prepropeptide); GDF15 (growth differentiation factor 15); BCL11A (B-cell CLL/lymphoma 11A (zinc finger protein)); CSRP3 (cysteine and glycine-rich protein 3 (cardiac LIM protein)); CXCL2 (chemokine (C-X-C motif) ligand 2); TOMM40 (translocase of outer mitochondrial membrane 40 homolog (yeast)); KCNK6 (potassium channel subfamily K member 6); KCNN2 (potassium intermediate/small conductance calcium-activated channel subfamily N member 2); SLC6A12 (solute carrier family 6 (neurotransmitter transporter betaine/GABA) member 12); ALOXE3 (arachidonate lipoxygenase 3); SOST (sclerosteosis); PRLHR (prolactin releasing hormone receptor); TIMM44 (translocase of inner mitochondrial membrane 44 homolog (yeast)); KCNN1 (potassium intermediate/small conductance calcium-activated channel subfamily N member 1); CHRNA9 (cholinergic receptor nicotinic alpha 9); GPC5 (glypican 5); GPR37 (G protein-coupled receptor 37 (endothelin receptor type B-like)); NKX2-1 (NK2 homeobox 1); HMMR (hyaluronan-mediated motility receptor (RHAMM)); PKHD1 (polycystic kidney and hepatic disease 1 (autosomal recessive)); AOC2 (amine oxidase copper containing 2 (retina-specific)); KRT20 (keratin 20); CORIN (corin serine peptidase); AZU1 (azurocidin 1); MAPK6 (mitogen-activated protein kinase 6); PAEP (progestagen-associated endometrial protein); CACNA2D4 (calcium channel voltage-dependent alpha 2/delta subunit 4); EIF3A (eukaryotic translation initiation factor 3 subunit A); BTG2 (BTG family member 2); P2RY14 (purinergic receptor P2Y G-protein coupled 14); PDLIM7 (PDZ and LIM domain 7 (enigma)); CACNA2D3 (calcium channel voltage-dependent alpha 2/delta subunit 3); LAMP3 (lysosomal-associated membrane protein 3); PLCL2 (phospholipase C-like 2); NOSIP (nitric oxide synthase interacting protein); CRHBP (corticotropin releasing hormone binding protein); KLK5 (kallikrein-related peptidase 5); ADAM2 (ADAM metallopeptidase domain 2); SIRPA (signal-regulatory protein alpha); PMPCB (peptidase (mitochondrial processing) beta); GPC4 (glypican 4); MYH6 (myosin heavy chain 6 cardiac muscle alpha); CXCL9 (chemokine (C-X-C motif) ligand 9); KCNK5 (potassium channel subfamily K member 5); KCNK10 (potassium channel subfamily K member 10); NMU (neuromedin U); SCN4B (sodium channel voltage-gated type IV beta); CAMK1D (calcium/calmodulin-dependent protein kinase ID); COL8A2 (collagen type VIII alpha 2); RAB11FIP1 (RAB11 family interacting protein 1 (class I)); NDOR1 (NADPH dependent diflavin oxidoreductase 1); ZNF318 (zinc finger protein 318); P2RX2 (purinergic receptor P2X ligand-gated ion channel 2); UGT1A6 (UDP glucuronosyltransferase 1 family polypeptide A6); LEMD3 (LEM domain containing 3); UGT1A1 (UDP glucuronosyltransferase 1 family polypeptide A1); PDLIM3 (PDZ and LIM domain 3); KCTD12 (potassium channel tetramerisation domain containing 12); KCNK9 (potassium channel subfamily K member 9); DSE (dermatan sulfate epimerase); DSPP (dentin sialophosphoprotein); KCNT2 (potassium channel subfamily T member 2); NMUR2 (neuromedin U receptor 2); CHST6 (carbohydrate (N-acetylglucosamine 6-0) sulfotransferase 6); CCL28 (chemokine (C-C motif) ligand 28); SLPI (secretory leukocyte peptidase inhibitor); CCL1 (chemokine (C-C motif) ligand 1); KCNK15 (potassium channel subfamily K member 15); KCTD15 (potassium channel tetramerisation domain containing 15); ANKRD1 (ankyrin repeat domain 1 (cardiac muscle)); SIGMAR1 (sigma non-opioid intracellular receptor 1); SLCO2A1 (solute carrier organic anion transporter family member 2A1); MUC16 (mucin 16 cell surface associated); CNTNAP1 (contactin associated protein 1); LGR6 (leucine-rich repeat-containing G protein-coupled receptor 6); ASPN (asporin); PLCH2 (phospholipase C eta 2); PLCL1 (phospholipase C-like 1); AGFG1 (ArfGAP with FG repeats 1); HOXB8 (homeobox B8); KCNK12 (potassium channel subfamily K member 12); KCNK4 (potassium channel subfamily K member 4); KCNRG (potassium channel regulator); KCTD13 (potassium channel tetramerisation domain containing 13); KCNT1 (potassium channel subfamily T member 1); RNF19A (ring finger protein 19A); CIAPIN1 (cytokine induced apoptosis inhibitor 1); TNS3 (tensin 3); AMELX (amelogenin X-linked); CRBN (cereblon); MLN (motilin); CXCR1 (chemokine (C-X-C motif) receptor 1); NPBWR2 (neuropeptides B/W receptor 2); KCMF1 (potassium channel modulatory factor 1); KCNK7 (potassium channel subfamily K member 7); KCNV1 (potassium channel subfamily V member 1); KCTD5 (potassium channel tetramerisation domain containing 5); KCNV2 (potassium channel subfamily V member 2); KCNK13 (potassium channel subfamily K member 13); ERAP2 (endoplasmic reticulum aminopeptidase 2); KCTD2 (potassium channel tetramerisation domain containing 2); KCTD3 (potassium channel tetramerisation domain containing 3); KCNK17 (potassium channel subfamily K member 17); KCTD10 (potassium channel tetramerisation domain containing 10); KCTD7 (potassium channel tetramerisation domain containing 7); SCT (secretin); NGDN (neuroguidin EIF4E binding protein); MLNR (motilin receptor); MPZL2 (myelin protein zero-like 2); PRO11 (proline rich lacrimal 1); KCNK16 (potassium channel subfamily K member 16); KCTD9 (potassium channel tetramerisation domain containing 9); KCTD11 (potassium channel tetramerisation domain containing 11); KCTD8 (potassium channel tetramerisation domain containing 8); KCTD4 (potassium channel tetramerisation domain containing 4); KCTD6 (potassium channel tetramerisation domain containing 6); KCTD1 (potassium channel tetramerisation domain containing 1); NPVF (neuropeptide VF precursor); MAGIX (MAGI family member X-linked); MRGPRX4 (MAS-related GPR member X4); MRGPRD (MAS-related GPR member D); TET2 (tet oncogene family member 2); KCTD14 (potassium channel tetramerisation domain containing 14); GLYATL1 (glycine-N-acyltransferase-like 1); ZNF493 (zinc finger protein 493); ZNF429 (zinc finger protein 429); MRGPRE (MAS-related GPR member E); SUN2 (Sad1 and UNC84 domain containing 2); AMTN (amelotin); MRGPRF (MAS-related GPR member F); CDK20 (cyclin-dependent kinase 20); KCNU1 (potassium channel subfamily U member 1); GATS (GATS stromal antigen 3 opposite strand); GLRA4 (glycine receptor alpha 4); IGHE (immunoglobulin heavy constant epsilon); DRGX (dorsal root ganglia homeobox); MRGPRG (MAS-related GPR member G); LOC729977 (hypothetical LOC729977); MT-TK (mitochondrially encoded tRNA lysine); LOC400680 (hypothetical gene supported by AK097381; BC040866); COP (clathrin-ordered protein); IGES (immunoglobulin E concentration serum); MGS (Mungen syndrome); TRNAS-AGA (transfer RNA serine (anticodon AGA)); and LOCI 00132258 (similar to secretory carrier membrane protein 2).

Non-limiting examples of taste-related genes include TAS2R38 (taste receptor, type 2, member 38); TAS1R1 (taste receptor, type 1, member 1); TAS2R3 (taste receptor, type 2, member 3); TAS2R5 (taste receptor, type 2, member 5); TAS2R1 (taste receptor, type 2, member 1); TAS2R16 (taste receptor, type 2, member 16); TAS2R4 (taste receptor, type 2, member 4); TAS2R14 (taste receptor, type 2, member 14); TAS2R10 (taste receptor, type 2, member 10); TAS2R7 (taste receptor, type 2, member 7); TAS2R13 (taste receptor, type 2, member 13); TAS2R9 (taste receptor, type 2, member 9); TAS2R8 (taste receptor, type 2, member 8); TAS1R3 (taste receptor, type 1, member 3); TAS2R31 (taste receptor, type 2, member 31); TAS1R2 (taste receptor, type 1, member 2); TAS2R43 (taste receptor, type 2, member 43); TAS2R50 (taste receptor, type 2, member 50); TAS2R46 (taste receptor, type 2, member 46); TAS2R30 (taste receptor, type 2, member 30); TAS2R42 (taste receptor, type 2, member 42); PLCB2 (phospholipase C, beta 2); TAS2R20 (taste receptor, type 2, member 20); TAS2R19 (taste receptor, type 2, member 19); GNG13 ((guanine nucleotide binding protein (G protein)), gamma 13); TAS2R12 (taste receptor, type 2, member 12 pseudogene); GNAT1 (guanine nucleotide binding protein (G protein), alpha transducing activity polypeptide 1); TAS2R41 (taste receptor, type 2, member 41); TAS2R60 (taste receptor, type 2, member 60); TAS2R40 (taste receptor, type 2, member 40); TAS2R39 (taste receptor, type 2, member 39); GCG (glucagon); TAS2R18 (taste receptor, type 2, member 18 pseudogene); GRM4 (glutamate receptor, metabotropic 4); LCN1 (lipocalin 1 (tear prealbumin)); TRPV1 (transient receptor potential cation channel, subfamily V, member 1); ACCN1 (amiloride-sensitive cation channel 1, neuronal); TAS2R45 (taste receptor, type 2, member 45); TAS2R15 (taste receptor, type 2, member 15 pseudogene); FOS (murine osteosarcoma viral oncogene homolog); SLC9A1 (solute carrier family 9 (sodium/hydrogen exchanger), member 1); INS (insulin); ACCN5 (amiloride-sensitive cation channel 5, intestinal); TAS2R2 (taste receptor, type 2, member 2 pseudogene); GRM7 (glutamate receptor, metabotropic 7); NPY (neuropeptide Y); LEP (leptin); CASR (calcium-sensing receptor); GNAZ (guanine nucleotide binding protein (G protein), alpha z polypeptide); CIB1 (calcium and integrin binding 1 (calmyrin)); ADCY10 (adenylate cyclase 10 (soluble)); LEPR (leptin receptor); DRD1 (dopamine receptor D1); LGR6 (leucine-rich repeat-containing G protein-coupled receptor 6); GRM8 (glutamate receptor, metabotropic 8); GRM6 (glutamate receptor, metabotropic 6); GLP1R (glucagon-like peptide 1 receptor); AGER (advanced glycosylation end product-specific receptor); SLC2A2 (solute carrier family 2 (facilitated glucose transporter), member 2); GIP (gastric inhibitory polypeptide); REN (rennin); PDYN (prodynorphin); RRBP1 (ribosome binding protein 1 homolog 180 kDa (dog)); SLC15A1 (solute carrier family 15 (oligopeptide transporter), member 1); OXT (oxytocin, prepropeptide); IL411 (interleukin 4 induced 1); VN1R17P (vomeronasal 1 receptor 17 pseudogene); TAS2R62P (taste receptor, type 2, member 62, pseudogene); TAS2R64P (taste receptor, type 2, member 64 pseudogene); TAS2R63P (taste receptor, type 2, member 63 pseudogene); PS5 (bitter taste receptor pseudogene PS5); PS3 (bitter taste receptor PS3); PS7 (bitter taste receptor Ps7 pseudogene); C6orf15 (chromosome 6 open reading frame 15); TAS2R6 (taste receptor, type 2, member 6); TAS2R22 (taste receptor, type 2, member 22); TAS2R33 (taste receptor, type 2, member 33); TAS2R37 (taste receptor, type 2, member 37); TAS2R36 (taste receptor, type 2, member 36); GNAT3 (guanine nucleotide binding protein, alpha transducing 3); TRPM5 (transient receptor potential cation channel, subfamily M, member 5); TRPM7 (transient receptor potential cation channel, subfamily M, member 7); GNB1 (guanine nucleotide binding protein (G protein), beta polypeptide 1); ITPR3 (inositol 1,4,5-triphosphate receptor, type 3); ACE (angiotensin I converting enzyme (peptidyl-dipeptidase A) 1); ENO2 (enolase 2 (gamma, neuronal)); CALCA (calcitonin-related polypeptide alpha); CCK (cholecystokinin); RTP3 (receptor (chemosensory) transporter protein 3); PL-5283 (PL-5283 protein); PRKCG (protein kinase C, gamma); KCNQ1 (potassium voltage-gated channel, KQT-like subfamily, member 1); BDNF (brain-derived neurotrophic factor); SCNN1A (sodium channel, nonvoltage-gated 1 alpha); GNB3 (guanine nucleotide binding protein (G protein), beta polypeptide 3); SCNN1B (sodium channel, nonvoltage-gated 1, beta); SCNN1G (sodium channel, nonvoltage-gated 1, gamma); GNB4 (guanine nucleotide binding protein (G protein), beta polypeptide 4); PDE1A (phosphodiesterase 1A, calmodulin-dependent); DMBT1 (deleted in malignant brain tumors 1); PDE3B (phosphodiesterase 3B, cGMP-inhibited); PDE1C (phosphodiesterase 1C, calmodulin-dependent 70 kDa); PRKCA (protein kinase C, alpha); NTRK3 (neurotrophic tyrosine kinase, receptor, type 3); NTRK2 (neurotrophic tyrosine kinase, receptor, type 2); PRKCQ (protein kinase C, theta); PRKACA (protein kinase, cAMP-dependent, catalytic, alpha); CCKBR (cholecystokinin B receptor); PRKCZ (protein kinase C, zeta); TH (tyrosine hydroxylase); NGFR (nerve growth factor receptor (TNFR superfamily, member 16)); DRD2 (dopamine receptor D2); NOS1 (nitric oxide synthase 1 (neuronal)); PRKCE (protein kinase C, epsilon); PRKCH (protein kinase C, eta); PRKCD (protein kinase C, delta); ABCB1 (ATP-binding cassette, sub-family B (MDR/TAP), member 1); MAPK1 (mitogen-activated protein kinase 1); PLCB3 (phospholipase C, beta 3 (phosphatidylinositol-specific)); ADCY8 (adenylate cyclase 8 (brain)); ADRBK2 (adrenergic, beta, receptor kinase 2); PRKACB (protein kinase, cAMP-dependent, catalytic, beta); PRKCI (protein kinase C, iota); CCKAR (cholecystokinin A receptor); KCNK3 (potassium channel, subfamily K, member 3); PLCB1 (phospholipase C, beta 1 (phosphoinositide-specific)); ADCY3 (adenylate cyclase 3); NTF3 (neurotrophin 3); PLCB4 (phospholipase C, beta 4); GNB5 (guanine nucleotide binding protein (G protein), beta 5); GNAL (guanine nucleotide binding protein (G protein), alpha activating activity polypeptide, olfactory type); GNB2 (guanine nucleotide binding protein (G protein), beta polypeptide 2); KCNK1 (potassium channel, subfamily K, member 1); HTR1A (5-hydroxytryptamine (serotonin) receptor 1A); CNGA3 (cyclic nucleotide gated channel alpha 3); PRKACG (protein kinase, cAMP-dependent, catalytic, gamma); PRKCB (protein kinase C, beta); RBP4 (retinol binding protein 4, plasma); GRP (gastrin-releasing peptide); PDE3A (phosphodiesterase 3A, cGMP-inhibited); KRT14 (keratin 14); SCNN1D (sodium channel, nonvoltage-gated 1, delta); PRKD1 (protein kinase D1); PDE1B (phosphodiesterase 1B, calmodulin-dependent); PDE2A (phosphodiesterase 2A, cGMP-stimulated); PRKD3 (protein kinase D3); SST (somatostatin); KCNK6 (potassium channel, subfamily K, member 6); KCNK2 (potassium channel, subfamily K, member 2); NTF4 (neurotrophin 4); GNG3 (guanine nucleotide binding protein (G protein), gamma 3); RNH1 (ribonuclease/angiogenin inhibitor 1); KCNK5 (potassium channel, subfamily K, member 5); KCNK10 (potassium channel, subfamily K, member 10); P2RX2 (purinergic receptor P2X, ligand-gated ion channel, 2); KCTD12 (potassium channel tetramerisation domain containing 12); KCNK9 (potassium channel, subfamily K, member 9); KCNT2 (potassium channel, subfamily T, member 2); KCNK15 (potassium channel, subfamily K, member 15); KCTD15 (potassium channel tetramerisation domain containing 15); KCNK12 (potassium channel, subfamily K, member 12); KCNK4 (potassium channel, subfamily K, member 4); KCNRG (potassium channel regulator); KCTD13 (potassium channel tetramerisation domain containing 13); KCNT1 (potassium channel, subfamily T, member 1); KCMF1 (potassium channel modulatory factor 1); KCNK7 (potassium channel, subfamily K, member 7); KCNV1 (potassium channel, subfamily V, member 1); KCTD5 (potassium channel tetramerisation domain containing 5); KCNV2 (potassium channel, subfamily V, member 2); KCNK13 (potassium channel, subfamily K, member 13); KCTD2 (potassium channel tetramerisation domain containing 2); KCTD3 (potassium channel tetramerisation domain containing 3); KCNK17 (potassium channel, subfamily K, member 17); KCTD10 (potassium channel tetramerisation domain containing 10); KCTD7 (potassium channel tetramerisation domain containing 7); KCNK16 (potassium channel, subfamily K, member 16); KCTD9 (potassium channel tetramerisation domain containing 9); KCTD11 (potassium channel tetramerisation domain containing 11); KCTD8 (potassium channel tetramerisation domain containing 8); KCTD4 (potassium channel tetramerisation domain containing 4); KCTD6 (potassium channel tetramerisation domain containing 6); KCTD1 (potassium channel tetramerisation domain containing 1); KCTD14 (potassium channel tetramerisation domain containing 14); RTP4 (receptor (chemosensory) transporter protein 4); KCNU1 (potassium channel, subfamily U, member 1); LOC730036 (hypothetical LOC730036); RPS6KA3 (ribosomal protein S6 kinase, 90 kDa, polypeptide 3); MAPT (microtubule-associated protein tau); CHEK2 (CHK2 checkpoint homolog (S. pombe)); FYN (FYN oncogene related to SRC, FGR, YES); APP (amyloid beta (A4) precursor protein); PTEN (phosphatase and tensin homolog); SOD1 (superoxide dismutase 1, soluble); CSTB (cystatin B (stefin B)); SHH (sonic hedgehog homolog (Drosophila)); AKR1B1 (aldo-keto reductase family 1, member B1 (aldose reductase)); COMT (catechol-β-methyltransferase); S100B (S100 calcium binding protein B); PTK2B (PTK2B protein tyrosine kinase 2 beta); PLCG2 (phospholipase C, gamma 2 (phosphatidylinositol-specific)); PSEN1 (presenilin 1); SLC6A3 (solute carrier family 6 (neurotransmitter transporter, dopamine), member 3); PAX6 (paired box 6); MMP1 (matrix metallopeptidase 1 (interstitial collagenase)); CACNA1A (calcium channel, voltage-dependent, P/Q type, alpha 1A subunit); CASP9 (caspase 9, apoptosis-related cysteine peptidase); PRKAR1A (protein kinase, cAMP-dependent, regulatory, type I, alpha (tissue specific extinguisher 1)); MMP3 (matrix metallopeptidase 3 (stromelysin 1, progelatinase)); ADCY6 (adenylate cyclase 6); CASP3 (caspase 3, apoptosis-related cysteine peptidase); GNAS (GNAS complex locus); MMP9 (matrix metallopeptidase 9 (gelatinase B, 92 kDa gelatinase, 92 kDa type IV collagenase)); NOTCH2 (Notch homolog 2 (Drosophila)); CREB1 (cAMP responsive element binding protein 1); SNCA (synuclein, alpha (non A4 component of amyloid precursor)); OPRM1 (opioid receptor, mu 1); CALM1 (calmodulin 1 (phosphorylase kinase, delta)); PLCG1 (phospholipase C, gamma 1); BRCA1 (breast cancer 1, early onset); APOE (apolipoprotein E); DBH (dopamine beta-hydroxylase (dopamine beta-monooxygenase)); PTGS2 (prostaglandin-endoperoxide synthase 2 (prostaglandin G/H synthase and cyclooxygenase)); ADRBK1 (adrenergic, beta, receptor kinase 1); ITGB4 (integrin, beta 4); NLGN3 (neuroligin 3); CD36 (CD36 molecule (thrombospondin receptor)); EEF2 (eukaryotic translation elongation factor 2); OPRD1 (opioid receptor, delta 1); HSPG2 (heparan sulfate proteoglycan 2); GAD1 (glutamate decarboxylase 1 (brain, 67 kDa)); ANXA1 (annexin A1); PRKAR2A (protein kinase, cAMP-dependent, regulatory, type II, alpha); HHEX (hematopoietically expressed homeobox); GRM1 (glutamate receptor, metabotropic 1); NPR1 (natriuretic peptide receptor A/guanylate cyclase A (atrionatriuretic), peptide receptor A); SYP (synaptophysin); CALM3 (calmodulin 3 (phosphorylase kinase, delta)); PRKAR2B (protein kinase, cAMP-dependent, regulatory, type II, beta); ADCY2 (adenylate cyclase 2 (brain)); SLC1A3 (solute carrier family 1 (glial high affinity glutamate transporter), member 3); GABBR1 (gamma-aminobutyric acid (GABA) B receptor, 1); PTPRS (protein tyrosine phosphatase, receptor type, S); KNG1 (kininogen 1); DDC (dopa decarboxylase (aromatic L-amino acid decarboxylase)); GNAQ (guanine nucleotide binding protein (G protein), q polypeptide); E2F4 (E2F transcription factor 4, p107/p130-binding); DRD4 (dopamine receptor D4); MAOA (monoamine oxidase A); CALM2 (calmodulin 2 (phosphorylase kinase, delta)); CHRNB2 (cholinergic receptor, nicotinic, beta 2 (neuronal)); GRK5 (G protein-coupled receptor kinase 5); PRLR (prolactin receptor); ID2 (inhibitor of DNA binding 2, dominant negative helix-loop-helix protein); TPH1 (tryptophan hydroxylase 1); PLCD1 (phospholipase C, delta 1); GNA11 (guanine nucleotide binding protein (G protein), alpha 11 (Gq class)); GNA12 (guanine nucleotide binding protein (G protein) alpha 12); CRH (corticotropin releasing hormone); GNRH1 (gonadotropin-releasing hormone 1 (luteinizing-releasing hormone)); S100A8 (S100 calcium binding protein A8); CYCS (cytochrome c, somatic); KCNB1 (potassium voltage-gated channel, Shal-related subfamily, member 1); DST (dystonin); ADCY1 (adenylate cyclase 1 (brain)); CHGA (chromogranin A (parathyroid secretory protein 1)); HTR3A (5-hydroxytryptamine (serotonin) receptor 3A); GAL (galanin prepropeptide); TACR3 (tachykinin receptor 3); ALDH7A1 (aldehyde dehydrogenase 7 family, member A1); PRKAR1B (protein kinase, cAMP-dependent, regulatory, type I, beta); AQP5 (aquaporin 5); AQP2 (aquaporin 2 (collecting duct)); AQP1 (aquaporin 1 (Colton blood group)); GLI3 (GLI family zinc finger 3); POU2F1 (POU class 2 homeobox 1); OTX2 (orthodenticle homeobox 2); TTR (transthyretin); CACNA1B (calcium channel, voltage-dependent, N type, alpha 1B subunit); IKBKAP (inhibitor of kappa light polypeptide gene enhancer in B-cells, kinase complex-associated protein); RHO (rhodopsin); UGT2B7 (UDP glucuronosyltransferase 2 family, polypeptide B7); LCT (lactase); TCOF1 (Treacher Collins-Franceschetti syndrome 1); KCNJ1 (potassium inwardly-rectifying channel, subfamily J, member 1); VIP (vasoactive intestinal peptide); AQP3 (aquaporin 3 (Gill blood group)); TAC1 (tachykinin, precursor 1); ADCY4 (adenylate cyclase 4); HP (haptoglobin); ALDH4A1 (aldehyde dehydrogenase 4 family, member A1); GDI1 (GDP dissociation inhibitor 1); SOX2 (SRY (sex determining region Y)-box 2); NOG (noggin); FST (follistatin); NDST1 (N-deacetylase/N-sulfotransferase (heparan glucosaminyl) 1); ABLIM1 (actin binding LIM protein 1); NOS2 (nitric oxide synthase 2, inducible); EIF2B1 (eukaryotic translation initiation factor 2B, subunit 1 alpha, 26 kDa); CA6 (carbonic anhydrase VI); DKK1 (dickkopf homolog 1 (Xenopus laevis)); SIX3 (SIX homeobox 3); SIX1 (SIX homeobox 1); HTT (huntingtin); AGRP (agouti related protein homolog (mouse)); NCAM2 (neural cell adhesion molecule 2); BBS4 (Bardet-Biedl syndrome 4); GNA15 (guanine nucleotide binding protein (G protein), alpha 15 (Gq class)); GNA13 (guanine nucleotide binding protein (G protein), alpha 13); ASCL1 (achaete-scute complex homolog 1 (Drosophila)); MGLL (monoglyceride lipase); PLCD3 (phospholipase C, delta 3); CEBPB (CCAAT/enhancer binding protein (C/EBP), beta); BBS1 (Bardet-Biedl syndrome 1); HES1 (hairy and enhancer of split 1, (Drosophila)); GNG2 (guanine nucleotide binding protein (G protein), gamma 2); TPH2 (tryptophan hydroxylase 2); P2RX3 (purinergic receptor P2X, ligand-gated ion channel, 3); AQP7 (aquaporin 7); CNGB1 (cyclic nucleotide gated channel beta 1); GABRR1 (gamma-aminobutyric acid (GABA) receptor, rho 1); GBX2 (gastrulation brain homeobox 2); SLC6A1 (solute carrier family 6 (neurotransmitter transporter, GABA), member 1); PEBP1 (phosphatidylethanolamine binding protein 1); KRT13 (keratin 13); NAV2 (neuron navigator 2); BBS2 (Bardet-Biedl syndrome 2); PLCD4 (phospholipase C, delta 4); CLDN8 (claudin 8); CLDN7 (claudin 7); CISH (cytokine inducible SH2-containing protein); GNGT2 (guanine nucleotide binding protein (G protein), gamma transducing activity polypeptide 2); GNG4 (guanine nucleotide binding protein (G protein), gamma 4); GNA14 (guanine nucleotide binding protein (G protein), alpha 14); UCN (urocortin); PDE4A (phosphodiesterase 4A, cAMP-specific (phosphodiesterase E2 dunce homolog, Drosophila)); MKKS (McKusick-Kaufman syndrome); GAST (gastrin); PRKX (protein kinase, X-linked); CHRD (chordin); PRSS2 (protease, serine, 2 (trypsin 2)); KRT20 (keratin 20); CLDN6 (claudin 6); CLCN4 (chloride channel 4); DLX5 (distal-less homeobox 5); TRPA1 (transient receptor potential cation channel, subfamily A, member 1); TRPM8 (transient receptor potential cation channel, subfamily M, member 8); PLCZ1 (phospholipase C, zeta 1); SLC5A2 (solute carrier family 5 (sodium/glucose cotransporter), member 2); GDF11 (growth differentiation factor 11); BLVRB (biliverdin reductase B (flavin reductase (NADPH))); SCN7A (sodium channel, voltage-gated, type VII, alpha); PANX1 (pannexin 1); IF135 (interferon-induced protein 35); NRAP (nebulin-related anchoring protein); HESS (hairy and enhancer of split 5 (Drosophila)); GSC (goosecoid homeobox); REEP1 (receptor accessory protein 1); CCL28 (chemokine (C-C motif) ligand 28); GJB4 (gap junction protein, beta 4, 30.3 kDa); B3GNT2 (UDP-GlcNAc:betaGal beta-1,3-N-acetylglucosaminyltransferase 2); CNGA2 (cyclic nucleotide gated channel alpha 2); ZNF423 (zinc finger protein 423); HESX1 (HESX homeobox 1); CNGA4 (cyclic nucleotide gated channel alpha 4); GPR158 (G protein-coupled receptor 158); MAGEL2 (MAGE-like 2); UBR3 (ubiquitin protein ligase E3 component n-recognin 3 (putative)); NPTXR (neuronal pentraxin receptor); SLC24A6 (solute carrier family 24 (sodium/potassium/calcium exchanger), member 6); GPRC6A (G protein-coupled receptor, family C, group 6, member A); SLC24A3 (solute carrier family 24 (sodium/potassium/calcium exchanger), member 3); BEST2 (bestrophin 2); OR8D2 (olfactory receptor, family 8, subfamily D, member 2); OR5P2 (olfactory receptor, family 5, subfamily P, member 2); FOXG1 (forkhead box G1); OR8B8 (olfactory receptor, family 8, subfamily B, member 8); OR8D1 (olfactory receptor, family 8, subfamily D, member 1); OR10A5 (olfactory receptor, family 10, subfamily A, member 5); OMP (olfactory marker protein); TFAP2E (transcription factor AP-2 epsilon (activating enhancer binding protein 2, epsilon); OR5P3 (olfactory receptor, family 5, subfamily P, member 3); OR10A4 (olfactory receptor, family 10, subfamily A, member 4); DMRTA1 (DMRT-like family A1); TMEM147 (transmembrane protein 147); OR8A1 (olfactory receptor, family 8, subfamily A, member 1); EBF2 (early B-cell factor 2); PKD1L3 (polycystic kidney disease 1-like 3); GPR179 (G protein-coupled receptor 179); RTP1 (receptor (chemosensory) transporter protein 1); KLHL35 (kelch-like 35 (Drosophila)); RGS21 (regulator of G-protein signaling 21); RTP2 (receptor (chemosensory) transporter protein 2); ACSM4 (acyl-CoA synthetase medium-chain family member 4); GUCY2E (guanylate cyclase 2E); CYP2G1P (cytochrome P450, family 2, subfamily G, polypeptide 1 pseudogene); OR7E35P (olfactory receptor, family 7, subfamily E, member 35 pseudogene); and NUDT16P1 (nudix (nucleoside diphosphate linked moiety X-type motif 16, pseudogene 1).

Exemplary sensory-related chromosomal sequences include TRPM7 (transient receptor potential cation channel, subfamily M, member 7); TRPM5 (transient receptor potential cation channel, subfamily M, member 5); TRPC5 (transient receptor potential cation channel subfamily C member 5); TRPC6 (transient receptor potential cation channel subfamily C member 6); TRPC1 (transient receptor potential cation channel subfamily C member 1); CNR1 (cannabinoid receptor 1 (brain)); CNR2 (cannabinoid receptor 2 (macrophage)); ADRBK1 (adrenergic beta receptor kinase 1); TRPA1 (transient receptor potential cation channel subfamily A member 1); POMC (proopiomelanocortin); CALCA (CGRP, calcitonin-related polypeptide alpha); CRF (CRH, corticotrophin releasing factor); PKA such as PRKACA (protein kinase cAMP-dependent catalytic alpha), PRKACB (protein kinase cAMP-dependent catalytic beta), PRKAR1A (protein kinase cAMP-dependent regulatory type 1 alpha (tissue specific extinguisher 1)), and PRKAR2A (protein kinase cAMP-dependent regulatory type II alpha); ERAL1 (Era G-protein-like 1 (E. coli)); NR2B (GRIN2B, glutamate receptor ionotropic N-methyl D-aspartate 2B); LGALS1 (lectin galactoside-binding soluble 1); TRPV1 (transient receptor potential cation channel subfamily V member 1); SCN9A (sodium channel voltage-gated type IX alpha subunit); OPRD1 (opioid receptor delta 1); OPRK1 (opioid receptor kappa 1); and OPRM1 (opioid receptor mu 1).

In certain embodiments, an animal created by a method of the invention may be used to study the effects of a mutation on the animal and on sensory disorders.

A further aspect of the present disclosure encompasses a method for assessing an indication of a sensory disorder in an animal model, wherein the animal model comprises a genetically modified animal comprising at least one edited chromosomal sequence encoding a sensory-related protein. This method includes comparing a selected parameter obtained from the animal model to the selected parameter obtained from a wild-type animal. Non-limiting examples of the selected parameter used for assessing at least one indication of a sensory disorder include a) spontaneous behaviors; b) performance during behavioral testing; c) physiological anomalies; d) abnormalities in tissues or cells; e) biochemical function; f) molecular structures; and combinations thereof.

The sensory disorders assessed by the method may include any one or more of the nociception disorders and taste disorders associated with the genes described above. Non-limiting examples of nociception disorders include allodynia; neuralgia; HSAN-1 such as hereditary sensory radicular neuropathy, ulcero-mutilating neuropathy, thevenard syndrome, familial trophoneurosis, mal perforant du pied, familial syringomyelia, and Charcot-Marie-Tooth type 2B syndrome; HSAN-2 such as congenital sensory neuropathy or Morvan's disease; HSAN-3 such as familial dysautonomia (FD) or Riley-Day syndrome; HSAN-4 such as congenital insensitivity to pain with anhidrosis (CIPA); and HSAN-5 such as congenital insensitivity to pain with partial anhidrosis. Non-limiting examples of taste disorders include dysgeusia, hypogeusia, and ageusia.

An indication of the sensory disorder may occur spontaneously in the animal model, or may be promoted by exposure to an exogenous agent including but not limited to a nociception stimulus, a taste stimulus, a sensory-related protein, a sensory-related agonist, and a sensory-related antagonist.

### C. ABC Transporters

ABC transporter proteins are a large and important superfamily of membrane transport proteins, ubiquitous in the animal kingdom. These transmembrane proteins hydrolyze ATP and use the energy to power various other functions, including translocation of molecules across intracellular and extracellular membranes, often against a concentration gradient. (For reviews, see Higgins, C. F., ABC transporters: from microorganisms to man, Annu. Rev. Cell Biol. 8 67-113 (1992); and M. Dean, Human ABC Transporter Superfamily, Bethesda (MD): National Center for Biotechnology Information (US); Nov. 18, 2002, available online at www.ncbi.nlm.nih.gov/bookshelf/br.fcgi?book=mono_{—}001).

In one embodiment, a method of the invention may be used to create an animal or cell in which at least one chromosomal sequence associated with an ABC transporter has been edited. Suitable chromosomal edits may include, but are not limited to, the type of edits detailed in section I(f) above.

An ABC transporter chromosomal sequence may encode an ABC transporter protein or may be an ABC transporter control sequence. An ABC transporter sequence may typically be selected based on an experimental association of the ABC transporter sequence to an animal disease or condition, especially a mammalian (e.g., a human) disease or condition. For example, the expression of an ABC transporter protein in a particular tissue may be elevated or depressed in a population having an ABC transporter-related disease or condition relative to a population lacking the disease or condition. Differences in protein levels may be assessed using proteomic or genomic analysis techniques known in the art.

Non-limiting examples of human ABC transporter genes include: ABCA1 (ABC1), ABCA2 (ABC2), ABCA3 (ABC3), ABCC, ABCA4 (ABCR), ABCA5, ABCA6, ABCA7, ABCA8, ABCA9, ABCA10, ABCA12, ABCA13, ABCB1 (PGY1, MDR), ABCB2 (TAP1), ABCB3 (TAP2), ABCB4 (PGY3), ABCB5, ABCB6 (MTABC), ABCB7 (ABC7), ABCB8 (MABC1), ABCB9, ABCB10 (MTABC2), ABCB11 (SPGP), ABCC1 (MRP1), ABCC2 (MRP2), ABCC3 (MRP3), ABCC4 (MRP4), ABCC5 (MRP5), ABCC6 (MRP6), CFTR (ABCC7), ABCC8 (SUR), ABCC9(SUR2), ABCC10 (MRP7), ABCC11 (ABCC12), ABCD1 (ALD), ABCD2 (ALDL1, ALDR), ABCD3(PXMP1, PMP70), ABCD4 (PMP69, P70R), ABCE1 (OABP, RNS4I), ABCF1 (ABC50), ABCF2 (ABCF3), ABCG1 (ABC8, White), ABCC2 (ABCP, MXR, BCRP), ABCC4 (White2), ABCC5 (White3), and ABCC8.

Non-limiting examples of mouse ABC transporter genes include Abca1, Abca2, Abca3, Abca4, Abca5, Abca6, Abca7, Abca8a, Abca8b, Abca9, Abca12, Abca13, Abcb1a, Abcb1b, Abcb2 (Tap1), Abcb3 (Tap2), Abcb4, Abcb5, Abcb6, Abcb7, Abcb8, Abcb9, Abcb10, Abcb11, Abcc1, Abcc2, Abcc3, Abcc4, Abcc5, Abcc6, Abcc7 (Cftr), Abcc8, Abcc9, Abcc10, Abcc11, Abcd1, Abcd2, Abcd3, Abcd4, Abce1, Abcf1, Abcf2, Abcf3, Abcg1, Abcg2, Abcg3, Abcg4, Abcg5 and Abcg8.

The Drosophila genome includes 56 ABC transporter genes with at least one representative of each of the known mammalian subfamilies. Non-limiting examples of Drosophilan ABC transporter genes include: G3156 (AAF45509, AE003417); CG2759 (w; AAF45826; AE003425); CG1703 (AAF48069; AE003486); CG1824 (AAF48177; AE003489); CG9281 (AAF48493; AE003500); CG8473 (AAF48511; AE003500); CG12703 (AE003513; AE003513); CG1819 (AAF50847; AE003569); CG1718 (AAF50837; AE003568); CG1801 (AAF50836; AE003568); CG1494 (AAF50838; AE003568); CG3164 (AAF51548; AE003590); CG4822 (AAF51551; AE003590); CG17646 (AAF51341; AE003585); CG9892 (AAF51223; AE003582); CG9664 (AAF51131; AE003580); CG9663 (AAF51130; AE00358); CG3327 (AAF51122; AE003580); CG2969 (Atet; AAF51027; AE003576); CG11147 (AAF52284; AE003611); CG7806 (AAF52639; AE003620); CG7627 (AAF52648; AE003620); CG5853 (AAF52835; AE003626); CG5772 (Sur; AAF52866; AE003627); CG6214 (AAF53223; AE003637); CG7491 (AAF53328; AE003641); CG17338 (AAF53736; AE003661); CG10441 (AAF53737; AE003661); CG9270 (AAF53950; AE003668); CG8799 (AAF58947; AE003833); CG3879 (Mdr49 AAF58437; AE003820); CG8523 (Mdr50; AAF58271; AE003815); CG8908 (AAF57490; AE003792); CG10505 (AAF46706; AE003453); CG17632 (bw; AAF47020; AE003461); CG7955 (AAF47526; AE003472); CG10226 (AAF50670; AE003563); Mdr65 (AAF50669; AE003563); CG5651 (AAF50342; AE003553); CG7346 (AAF50035; AE003544); CCG4314 (st; AAF49455; AE003527); CG5944 (AAF49305; AE003522); CG6052 (AAF49312; AE003523); CG9330 (AAF49142; AE003516); CG14709 (AAF54656; AE003692); CG4225 (AAF55241; AE003710); CG4562 AAF55707; AE003728); CG4794 (AAF55726; AE003728); CG5789 (AAF56312; AE003748); CG18633 (AAF56360; AE003749); CG11069 (AAF56361; AE003749); CG6162 (AAF56584; AE003756); CG9990 (AAF56807; AE003766); CG11898 (AAF56870; AE003768); CG11897 (AAF56869; AE003768); and CG2316 (AAF59367; AE003844).

Exemplary ABC transporter proteins include MDR1, BCRP (ABCG2), MRP1 (ABCC2) and MRP2 (ABCC2), and their mouse homologs Mdr1a (Abcb1a), Mdr1b (Abcb1b), Bcrp (Abcg2), Mrp1 (Abcc1), and Mrp2 (Abcc2), and any combination thereof. It should be understood that the gene designations as used herein, while referring to the human and mouse genomes, encompass the close homologs of any of these that have been identified among other animals including invertebrates such as C. elegans and D. melanogaster, and mammals, including but not limited to rats, hamsters, cats and dogs. Close homologs can be identified by sequence analysis, phylogenetic analysis, functional assays, or any combination thereof.

In certain embodiments, an animal created by a method of the invention may be used to study the effects of mutation on the animal or on ABC transporters.

v. Humanized Models

An animal created by a method of the invention may also be used as a humanized model. The humanized model expresses a human nucleic acid sequence in a non-human animal, as detailed above. In one embodiment, a research application or model described in section II(a) may be humanized. In another embodiment, a livestock animal or a companion animal as described below may be humanized.

### (b) Livestock Applications

In an embodiment, a method of the invention may be used to create a livestock animal with one or more chromosomal edits that result in one or more desirable traits. As used herein, “livestock animal” refers to an animal that may be raised for profit. Non-limiting examples of livestock animals are listed in this section and described in detail below.

Non-limiting examples of desirable traits in a livestock animal include a particular coat color or texture, disease resistance, increased fertility, increased meat production, increased muscle to fat ratio, increased milk production, reduced excrement pollution, etc. In another embodiment, a method of the invention may be used to create a livestock animal with a chromosomal edit in a gene listed in Table D.

TABLE D

| ANIMAL | GENE |
|---|---|
| Equine | HYPP; HERDA; EDNRB; Gray; Champagne Dilution; |
| | Tobiano; Red/Black Factor; Agouti; Sabino1; MATP; Silver |
| | Dilution |
| Ovine | Phen ID 424; Phen ID 2821; Phen ID 2555; Tyrosinase |
| (Sheep) | related protein (Trp1); Scrapie resistant protein (PrP) |
| Porcine | Myostatin/GDF8; CD163; Sialoadhesin; Halothane; |
| (Pig) | Estrogen receptor (ESR); Insulin-like growth factor 2 (IGF2); |
| | Growth hormone related hormone (GHRH); Phytase; |
| | Napole gene (RN) |
| Bovine | Pharmaceuticals; Caseins; Trait-associated SNPs; TBD; |
| (Cow) | Galactosidase, lactase; Beta Lactaglobulin; Alpha |
| | Lactalbumin (primary allergen and positive regulator of |
| | lactose); Alpha Lactalbumin (primary allergen and positive |
| | regulator of lactose); Osteopontin; Regeneration inducing |
| | peptide for tissues and cells (RIPTAC); Phene ID 2729, |
| | Group 001372; Casein; Casein; Acetyl CoA Carboxylase; |
| | Casein; Alpha Lactalbumin (primary allergen and positive |
| | regulator of lactose); Beta Casein and/or Kappa Casein; |
| | Casein; Lactase; Microsatellies TGLA49 and BM6438; |
| | FGFR3, EVC2; Growth hormone; Tyrosinase (Tyr), |
| | Tyrosinase-related protein 1 (Tyrp1), Melanocortin 1 |
| | receptor (MC1R), and Dopachrome tautomerase (Dct); |
| | Aggrecan (ACAN); Phene ID 2483, Group 001294; BST; |
| | Lactoferrin; Myostatin; Prion protein (PRP); Melanocortin 1 |
| | receptor (MC1R) |
| Silkworm | FibH (Fibroin) |
| Feline | Fel d1; TYR; Various Coat Color Genes; FGF5; Cauxin |

Additionally, in exemplary embodiments, a livestock animal may be an ovine, equine, bovine, or porcine animal, as detailed below.

i. Ovine Applications

In one embodiment, a method of the invention may be used to create an ovine or an ovine cell in which at least one chromosomal sequence has been edited. Non-limiting examples of ovine chromosomal sequences to be edited may include those that encode proteins associated with coat color, pattern, wool fiber structure, and disease resistance. For instance, non-limiting examples of proteins associated with coat color, pattern and/or wool fiber structure include MSH receptor proteins, agouti protein, Tyrosinase related proteins and keratin-associated proteins. Non-limiting examples of suitable coat color proteins include tyrosinase (TYR), tyrosinase-related protein 1 (TYRP1), agouti signaling protein (ASIP), and melanophilin (MLPH). Those of skill in the art appreciate that other proteins associated with coat color, coat pattern, and wool fiber structure exist, but the genetic loci encoding these other proteins have yet to be determined. Non-limiting examples of sequences involved in disease resistance include PRPN, which is associated with transmissible spongiform encephalopathy (TSE).

In one embodiment, a method of the invention may be used to create a genetically modified ovine comprising at least one edited chromosomal sequence that exhibits a phenotype desired by humans. For example, inactivation of the chromosomal sequence encoding Agouti may result in a sheep with a striped color. In other embodiments, the ovine animal comprising at least one edited chromosomal sequence may be used as a model to study the genetics of coat color, coat pattern, and/or hair growth. Additionally, an ovine animal comprising at least one disrupted chromosomal sequence may be used as a disease model to study a disease or condition that affects humans or other animals. Non-limiting examples of suitable diseases or conditions include albinism, hair disorders, and baldness. Additionally, the disclosed ovine cells and lysates of said cells may be used for similar research purposes.

ii. Equine Applications

In one embodiment, a method of the invention may be used to create an equine or an equine cell in which at least one chromosomal sequence has been edited. Non-limiting examples of equine chromosomal sequences to be edited include those that encode for coat color, pattern, and disease resistance.

Non-limiting examples of suitable coat color genes encoding proteins for coat color and pattern include Extension (Black/Red Factor), Agouti, MC1R, Gray Modifier, Champagne Dilution, Tobiano, Silver Dilution, MATP (Cream Dilution), Pearl Dilution, and Sabino1. Those of skill in the art appreciate that other genes and proteins may be associated with coat color and coat pattern, but the genetic loci have yet to be determined.

In a further embodiment, a method of the invention may be used to create a genetically modified equine that comprises an edited chromosomal sequence encoding HERDA, wherein the chromosomal sequence is inactivated such that certain alleles of HERDA protein are not produced. Furthermore, the genetically modified equine having inactivated HERDA variants of the chromosomal sequence described herein may exhibit reduced occurrence and transmission carriers of HERDA. In a non-limiting embodiment, the genetically modified equine may comprise an edited chromosomal sequence encoding HERDA. In another non-limiting embodiment, the genetically modified equine may comprise an edited chromosomal sequence inactivating HERDA only in the forms of variants that are known to be carriers.

In another embodiment, a method of the invention may be used to create a genetically modified equine that comprises an edited chromosomal sequence encoding HYPP, wherein the chromosomal sequence is inactivated such that the HYPP dominant allele is inactivated and the HYPP protein is not produced. Furthermore, the genetically modified equine having the inactivated HYPP dominant allele and chromosomal sequence described herein may exhibit reduced transmittal and perpetuation of HYPP in horses. In a non-limiting example, the genetically modified equine may comprise an edited chromosomal sequence encoding HYPP.

In yet another embodiment, a method of the invention may be used to create a genetically modified equine that comprises an edited chromosomal sequence encoding the Overo protein, wherein the chromosomal sequence is inactivated such that certain alleles of the Overo protein are not produced and/or are not fatal, but are still able to produce a frame overo phenotype. In a non-limiting example, the genetically modified equine animal may comprise an edited chromosomal sequence encoding Overo wherein the dominant allele is inactivated. In another non-limiting example, the genetically modified equine may comprise an edited chromosomal sequence inactivating Overo only in the forms of variants that are known to express lethal or harmful phenotypes. In yet another embodiment, the modification changes the dinucleotide TC->AG mutation to revert the mutation back to isoleucine in the EDNRB protein.

In a still further embodiment, a method of the invention may be used to create a genetically modified equine that comprises an edited chromosomal sequence encoding GBE, wherein the chromosomal sequence is inactivated such that the GBE recessive allele is inactivated and the corresponding protein is not produced. Furthermore, a genetically modified equine having the inactivated GBE variants may exhibit reduced occurrence and carriers of GBE. In a non-limiting example, the genetically modified equine may comprise an edited chromosomal sequence encoding GBE. In another non-limiting embodiment, the genetically modified equine may comprise an edited chromosomal sequence inactivating GBE only in the forms of variants that are known to be express lethal or harmful phenotypes.

In an alternative embodiment, a method of the invention may be used to create a genetically modified equine that may comprise an edited chromosomal sequence encoding JEB, wherein the chromosomal sequence is inactivated such that the JEB recessive allele is inactivated and JEB protein not produced. Furthermore, the genetically modified equine having the inactivated JEB variants may exhibit reduced occurrence and transmission of JEB. In a non-limiting example, the genetically modified equine may comprise an edited chromosomal sequence encoding JEB. In another non-limiting example, the genetically modified equine may comprise an edited chromosomal sequence inactivating JEB only in the forms of variants that are known to be carriers.

In another alternative embodiment, a method of the invention may be used to create a genetically modified equine that comprises an edited chromosomal sequence encoding PSSM, wherein the chromosomal sequence is inactivated such that the PSSM dominant allele and protein are not produced. Furthermore, the genetically modified equine having the inactivated PSSM dominant allele and chromosomal sequence described herein may exhibit reduced transmittal and perpetuation of PSSM in horses. In a non-limiting embodiment, the genetically modified equine may comprise an edited chromosomal sequence encoding PSSM.

In yet another alternative, a method of the invention may be used to create a genetically modified equine that comprises an edited chromosomal sequence including the C/C, C/T or T/T variant of myostatin for speed and athletic performance, depending on the nature of the desired phenotypic trait.

A method of the invention may also be used to create a genetically modified equine comprising any combination of the above described chromosomal alterations. For example, the genetically modified equine may comprise an inactivated agouti and/or edited PSSM chromosomal sequence, a modified MATP chromosomal sequence, and/or a modified or inactivated JEB chromosomal sequence.

An equine or equine cell detailed herein may have several applications. In one embodiment, a genetically modified equine comprising at least one edited chromosomal sequence may exhibit a phenotype desired by humans. For example, inactivation of the chromosomal sequence encoding Agouti may result in an equine with a striped color coat. In other embodiments, the equine comprising at least one edited chromosomal sequence may be used as a model to study the genetics of coat color, coat pattern, and/or hair growth. Additionally, an equine comprising at least one disrupted chromosomal sequence may be used as a model to study a disease or condition that affects humans or other animals (see section II(a) above). Non-limiting examples of suitable diseases or conditions include albinism, hair disorders, and baldness, in addition to skin diseases such as Hyperelastosis Cutis, or muscular diseases such as Hyperkalemic Periodic Paralysis Disease, Lethal White Overo Syndrome, Glycogen Branching Enzyme Deficiency disorder, and Polysaccharide Storage Myopathy, Recurrent exertional rhabdomyolysis (RER), Severe Combined Immunodeficiency Disorder (SCID). Additionally, the disclosed equine cells and lysates of said cells may be used for similar research purposes.

iii. Porcine Applications

In one embodiment, a method of the invention may be used to create a porcine or porcine cell in which at least one chromosomal sequence has been edited. Non-limiting examples of porcine chromosomal sequences to be edited and/or inserted may include those that code for coat color, pattern, disease resistance, meat quality, increased litter size, meat/fat ratio, and sequences that reduce phosphate pollution, such as phytase.

In some embodiments, a method of the invention may be used to create a porcine comprising a chromosomal edit in a nucleic acid sequence associated with coat color or pattern. Non-limiting examples of porcine chromosomal sequences that affect coat color or pattern include MC1R. The melanocortin receptor 1 (MC1R) plays a central role in regulation of eumelanin (black/brown) and phaeomelanin (red/yellow) synthesis within the mammalian melanocyte and is encoded by the classical Extension (E) coat color locus.

In another non-limiting embodiment, a method of the invention may be used to create a porcine comprising a chromosomal edit in a nucleic acid associated with disease resistance. Such a genetically modified porcine may comprise an edited chromosomal sequence such as CD163 or sialoadhesion.

In still another embodiment, a method of the invention may be used to create a porcine comprising a chromosomal edit in a nucleic acid associated with meat quality, meat quantity, and/or meat to fat ratio. For instance, non-limiting examples of porcine chromosomal sequences to be deleted or edited in porcine for increased muscle growth include those that code for proteins such as Myostatin/GDF8. Non-limiting examples of chromosomal sequences involved in meat quality include HAL, RN, or PSS. In yet another embodiment, the genetically modified porcine may comprise an edited chromosomal sequence encoding a sequence involved in meat/fat ratio, such as IGF2, GHRH, H-FABP, GH, IGF1, PIT1, GHRHR, GHR or combinations thereof.

In still yet another embodiment, a method of the invention may be used to create a porcine comprising a chromosomal edit in a nucleic acid associated with litter production. For instance, a genetically modified porcine may comprise an edited or modified chromosomal sequence encoding ESR for increased litter production.

In a further embodiment, a method of the invention may be used to create a porcine comprising a chromosomal edit in a nucleic acid associated with the reduction of phosphate pollution. For instance, a genetically modified porcine may comprise an edited chromosomal sequence encoding phytase for reduction of phosphate pollution.

The porcine animals and cells disclosed herein may have several applications. In one embodiment, the genetically modified porcine comprising at least one edited chromosomal sequence may exhibit a phenotype desired by humans. For example, modification of the chromosomal sequence encoding one of the MC1R alleles may result in porcine producing hair with desired coat color or pattern. In other embodiments, the porcine comprising at least one edited chromosomal sequence may be used as a model to study the genetics of coat color, coat pattern, and/or hair growth. Additionally, a porcine comprising at least one disrupted chromosomal sequence may be used as a model to study a disease or condition that affects humans or other animals (see section II(a) above). Non-limiting examples of suitable diseases or conditions include albinism, hair disorders, and baldness. Additionally, the disclosed porcine cells and lysates of said cells may be used for similar research purposes as detailed in section II(a) above.

iv. Bovine Applications

In one embodiment, a method of the invention may be used to create a bovine or bovine cell in which at least one chromosomal sequence has been edited. Non-limiting examples of bovine chromosomal sequences to be edited and/or inserted include those that code for proteins related to milk production, quality and processing, meat production and quality, coat color and quality, environmental impact, and breeding.

In certain embodiments, a method of the invention may be used to create a bovine with a chromosomal edit in a sequence associated with milk production, quality, and processing. For instance, a chromosomal sequence to be edited may include caseins, lactose and lactose-related proteins (e.g. galactosidase, lactase, galactose, beta lactaglobulin, alpha lactalbumin, lactoferrin), osteopontin, acetyl coA carboxylase, tyrosinases and related proteins, regeneration inducing peptide for tissues and cells (RIPTAC) and other growth hormones, proline rich polypeptide (PRP), alph-lactalbumin (LA), lactoperoxidase, and lysozyme.

In another embodiment, a method of the invention may be used to create a bovine with a chromosomal edit in a sequence associated with meat product and quality, for instance, such as FGFR3, EVC2, MC1R, and myostatin (mh).

In other embodiments, a method of the invention may be used to create a bovine with a chromosomal edit in a sequence associated with BSE-resistance (such as PRPN), coat color and quality (such as MC1R, TYRP1, MGF or KITLG), environmental impact, and breeding. In certain embodiments, the genetic loci have not necessarily been determined, but can be using methods commonly known in the art.

The bovine animals and cells disclosed herein may have several applications. In one embodiment, the genetically modified bovine comprising at least one edited chromosomal sequence may exhibit a phenotype desired by humans. For example, inactivation of the chromosomal sequence encoding Agouti may result in bovine having striped color coat. In other embodiments, the bovine comprising at least one edited chromosomal sequence may be used as a model to study the genetics of coat color, coat pattern, and/or hair growth. Additionally, a bovine comprising at least one disrupted chromosomal sequence may be used as a model to study a disease or condition that affects humans or other animals. Non-limiting examples of suitable diseases or conditions include albinism, hair disorders, and baldness. Additionally, the disclosed bovine cells and lysates of said cells may be used for similar research purposes.

### (c) Companion Animal Applications

In another embodiment, a method of the invention may be used to create a companion animal with one or more than one chromosomal edit that results in one or more desirable traits. As used herein, “companion animal” refers to an animal that is traditionally kept for non-profit purposes. Note, however, that in some instances companion animals may be bred for profit. Non-limiting examples of companion animals are detailed herein and in section III below. Non-limiting examples of suitable desirable traits in a companion animal include hypoallergenicity, a particular coat color or texture, disease resistance, reduced urine or feces odor, etc.

In certain embodiments, a method of the invention may be used to create a companion animal with a chromosomal edit in a gene listed in Table D above.

In exemplary embodiments, a method of the invention may be used to create a companion animal that comprises one or more than one chromosomal edit, such as a feline, a canine, or a rabbit. Each is discussed in more detail below.

i. Feline

In one embodiment, a method of the invention may be used to create a feline or feline cell in which at least one chromosomal sequence has been edited. Non-limiting examples of feline chromosomal sequences to be edited may include those that code for proteins such as allergen proteins, proteins involved in urine odor production, and proteins involved in coat color, coat pattern, and/or hair length.

In certain embodiments, a method of the invention may be used to create a feline with a chromosomal edit in a nucleic acid sequence associated with hypoallergenicity. Preferred allergen proteins include Felis domesticus 1 (Fel d1), which is the primary allergen present on cats and is a heterodimer of chain 1 and chain 2 peptides encoded by separate genes in the feline genome.

In another embodiment, a method of the invention may be used to create a feline with a chromosomal edit in a nucleic acid sequence associated with urine odor production. For instance, a chromosomal edit may be made in a protein involved in the production of urine odor, such as cauxin, which generates the major urinary pheromone felinine.

In yet another embodiment, a method of the invention may be used to create a feline with a chromosomal edit in a nucleic acid sequence associated with coat color, length, or pattern. Non-limiting examples of suitable coat color proteins include tyrosinase (TYR), tyrosinase-related protein 1 (TYRP1), augoti signaling protein (ASIP), and melanophilin (MLPH). A non-limiting example of a protein involved in hair length is fibroblast growth factor 5 (FGF5). Those of skill in the art appreciate that many other proteins are involved in coat color, coat pattern, and hair length, but their genetic loci have not been determined.

The animals and cells disclosed herein may have several applications. In one embodiment, the genetically modified feline comprising at least one edited chromosomal sequence may exhibit a phenotype desired by humans. For example, inactivation of the chromosomal sequence encoding Fel dl may result in cats that are hypoallergenic or non-allergenic. In other embodiments, the feline comprising at least one edited chromosomal sequence may be used as a model to study the genetics of coat color, coat pattern, and/or hair growth. Additionally, a feline comprising at least one disrupted chromosomal sequence may be used as a model to study a disease or condition that affects humans or other animals (see section II(a) above). Non-limiting examples of suitable diseases or conditions include albinism, deafness, skin disorders, hair disorders, and baldness. Additionally, the disclosed feline cells and lysates of said cells may be used for similar research purposes.

ii. Rabbit

In one embodiment, a method of the invention may be used to create a rabbit or rabbit cell in which at least one chromosomal sequence has been edited. Non-limiting examples of rabbit chromosomal sequences to be edited and/or inserted may include those associated with cardiovascular disease, immunodeficiency, and coat color, pattern and/or length.

In one embodiment, a method of the invention may be used to create a rabbit comprising one or more chromosomal edits in a sequence associated with cardiovascular disease. Non-limiting examples of rabbit chromosomal sequences associated with cardiovascular disease may include apo A, apoA-I, apoB, apoE2, apoE3 and lecithin-cholesterol acyltransferase (LCAT), as well as for rabbit apolipoprotein B, mRNA-editing enzyme catalytic poly-peptide 1 (APOBEC-1). Further non-limiting examples of rabbit chromosomal sequences to be edited include those that code for proteins relating to an autosomal dominant disease—Familial hypertrophic cardiomyopathy (FHC). FHC can be caused by multiple mutations in genes encoding various contractile, structural, channel and kinase proteins.

In another embodiment, a method of the invention may be used to create a rabbit comprising a chromosomal edit in a nucleic acid sequence associated with immunodeficiency. Non-limiting examples of rabbit chromosomal sequences to be edited may include fumarylacetoacetate hydrolase (FAH), recombination-activating genes-1 (Rag1), recombination-activating genes-1 (Rag2), Forkhead box O1 (Foxo1), DNAPK (dsDNA-dependent protein kinase), IL2 gamma receptor.

A method of the invention may also be used to create a genetically modified rabbit comprising any combination of the above described chromosomal alterations. For example, the genetically modified rabbit may comprise a modified or inactivated FAH, and/or modified or inactivated RAG1 chromosomal sequence, and/or a modified RAG2 chromosomal sequence, and/or a modified or inactivated Foxo1, DNAPK, and/or IL2 gamma receptor. All and any combination of the above described chromosomal alterations may be used for hepatocyte expansion either from human or other sources, which further enables drug metabolism studies, toxicology studies, safety assessment studies, infection disease research, chronic liver disease, acute liver disease, hepatocellular carcinoma, hepatitis, and any other liver infections or diseases.

In yet another embodiment, a method of the invention may be used to create a genetically modified rabbit that comprises an edited chromosomal sequence encoding Hairless homolog protein (hr). Rabbit that carry a mutation at hr locus may develop seemingly normal hair follicles (HF) but would shed its hairs completely soon after birth. The genetically modified rabbit comprising an edited hr chromosomal sequence may be used as a model organism for wound healing assays, skin irritation assays, treatment of viral infections, bacterial infections, crossing to other rabbit models, and for any application in which a normal rabbit would have to be shaved.

Additionally, a method of the invention may be used to create a genetically modified rabbit comprising any combination of the above described chromosomal alterations. For example, the genetically modified rabbit may comprise an inactivated ApoE, and/or FAH, and/or RAG1 chromosomal sequence, and/or a modified RAG2 chromosomal sequence, and/or a modified or inactivated Foxo1, DNAPK and/or IL2 gamma receptor, and/or hairless homolog protein chromosomal sequence.

The genetically modified rabbits and cells disclosed herein may have several applications. In one embodiment, the genetically modified rabbit comprising at least one edited chromosomal sequence may exhibit a phenotype desired by humans. For example, inactivation of the chromosomal sequence encoding Hairless homolog gene may result in rabbits that are hairless soon after birth, so that the rabbits do not need to be shaved as often required in various experimental use. In other embodiments, the rabbit comprising at least one edited chromosomal sequence may be used as a model to study the genetics of coat color, coat pattern, and/or hair growth, body size, bone development, and muscle development and structure. Additionally, a rabbit comprising at least one disrupted chromosomal sequence may be used as a model to study a disease or condition that affects humans, rabbits or other animals (see section II(a) above). Non-limiting examples of suitable diseases or conditions include cardiovascular diseases, ocular diseases, thyroid disease, autoimmune diseases, and immunodeficiency. Additionally, the disclosed rabbit cells and lysates of said cells may be used for similar research purposes.

iii. Canine

In one embodiment, a method of the invention may be used to create a canine or canine cell in which at least one chromosomal sequence has been edited. Non-limiting examples of canine chromosomal sequences to be edited and/or inserted include those that code for an allergy related protein, limb length, body size, coat color, pattern, and/or texture, and disease etc.

In certain embodiments, a method of the invention may be used to create a canine with one or more than one chromosomal edits in a nucleic acid sequence associated with hypoallergenicity. Non-limiting examples of such canine chromosomal sequences include Can f 1. A dog with Can f 1 “knock-out” or modification may be hypoallergic, or non-allergic, and/or without excessive barking.

In other embodiments, a method of the invention may be used to create a canine with one or more than one chromosomal edits in a nucleic acid sequence associated with limb length or body size. For instance, suitable nucleic acid sequences may include fibroblast growth factor-4 (FGF4) and insulin like growth factor-1 (IGF-1).

In another embodiment, a method of the invention may be used to create a canine with one or more than one chromosomal edits in a nucleic acid sequence associated with coat color, pattern, length, and/or texture. For instance, a suitable nucleic acid sequence may be associated with fur smooth versus wire texture (T-spondin-2, PSPO2 for wire hair), long versus short fur (fibroblast growth factor-5, FGF5), curly versus straight fur (keratin71, KRT71), hairless (fork head box transcription factor family, FOX13), coat color (melanocortin 1 receptor, McIr; Agouti; and β-defensin, CBD103), and complete or partial absence of pigmentation (microphthalmia-associated transcription factor, MITF). Those of skill in the art appreciate that other proteins are involved in coat color, coat pattern, and hair length, but the genetic loci have not been determined.

In a further embodiment, a method of the invention may be used to create a canine with one or more than one chromosomal edit in a nucleic acid sequence associated with a human disease. Non-limiting examples of such diseases include vision disorders, kidney cancer, narcolepsy, rheumatoid arthritis, SCID, keratin-associated diseases, cystinuria, bleeding disorders, ceroid lipofuscinosis and copper toxicosis. In one embodiment, the genetically modified canine may comprise an edited chromosomal sequence encoding hypocretin-2-receptor gene HCRTR2. In another embodiment, the chromosomal edit may be in the RCND locus. In yet another embodiment, a genetically modified canine may comprise an edited chromosomal sequence encoding protein folliculin. In still another embodiment, a genetically modified canine may comprise a chromosomal edit in the RPE65 gene.

A method of the invention may be used to create a genetically modified canine that comprises any combination of the above described chromosomal alterations. For example, the genetically modified canine may comprise an inactivated Can f 1 and/or Agouti chromosomal sequence, a modified FGF4 chromosomal sequence, and/or a modified or inactivated HCRTR2, RCND, and/or RPE65 chromosomal sequence.

The canine animals and cells created by a method of the invention may have several applications. In one embodiment, the genetically modified canine comprising at least one edited chromosomal sequence may exhibit a phenotype desired by humans. For example, inactivation of the chromosomal sequence encoding Can f 1 may result in dogs that are hypoallergenic or non-allergenic, and/or without excessive barking. In other embodiments, the canine comprising at least one edited chromosomal sequence may be used as a model to study the genetics of coat color, coat pattern, and/or hair growth, body size, leg length versus width, and skull shape. Additionally, a canine comprising at least one disrupted chromosomal sequence may be used as a model to study a disease or condition that affects humans, canines or other animals (see section II(a) above). Non-limiting examples of suitable diseases or conditions include cancer, deafness, heart disease, cataracts, hip dysplasia, thyroid disease, bloat, autoimmune diseases, progressive retinal atrophy, and epilepsy. Additionally, the disclosed canine cells and lysates of said cells may be used for similar research purposes.

### (d) Biomolecule Production Applications

In some embodiments, a method of the invention may be used to create an animal or a cell that produces a biomolecule. For instance, in one embodiment, a method of the invention may be used to create an animal or cell that comprises one or more chromosomal edits such that the animal or cell produces a biomolecule that the animal or cell would not typically produce absent the chromosomal edit. For instance, a method of the invention may be used to create a cell that produces an antibiotic. Or alternatively, a method of the invention may be used to create a bovine that produces a desired biomolecule in its milk, as detailed in section II(b) above.

An additional aspect of the invention encompasses a method of producing purified biological components using a genetically modified cell or animal comprising an edited chromosomal sequence encoding a protein. Non-limiting examples of such biological components include antibodies, cytokines, signal proteins, enzymes, receptor agonists and receptor antagonists.

In another embodiment, a method of the invention may be used to create an animal or cell that comprises one or more chromosomal edits such that the animal or cell produces a modified biomolecule compared to a biomolecule the animal or cell would typically produce. For instance, a method of the invention may be used to create a silkworm that comprises a chromosomal edit such that the silkworm produces silk that is more desirable. Non-limiting examples of silkworm chromosomal sequences to be edited include those that code for proteins specifically expressed in the silk gland. The silk gland is the site where silk proteins are synthesized and can be divided into three morphologically and functionally distinct compartments: ASG, MSG and PSG. In one embodiment, the genetically modified silkworm comprising modified silk fibroin proteins in PSG, including fibroin heavy chain (FibH), fibroin light chain (FibL) and fibrohexamerin P25 may have silk fiber of different phenotype in color, texture, smoothness, length, strength, weight or the ability to absorb dyes. In other embodiments, the genetically modified silkworm comprises a modified gene encoding the juvenile hormone binding protein which is involved in juvenile hormone signal transduction in the PSG and mediating the growth and development of the silk gland. Yet in another embodiment, a genetically modified silkworm comprises a modified serf gene in the MSG, which yields the glue protein sericin that is sticky and coats the outside of the silk strand over the fibroin protein core. Sericin comprises about 10-25% of silk, and has to be degummed during the silk processing. Genetically modified silkworm comprising a modified serf gene may produce silk fiber without the need of extensive degumming process. As a result, genetically modified silk fiber doesn't need the “weighting” practice by adding metals to silk fabric in textile manufacturing.

A non-limiting example of a group of proteins involved in silk production are transporters involved in transporting substances relative to silk formation, such as members of the solute carrier family (family 35 member B3, member E1, and family 39 member 9) and the transmembrane trafficking protein isoform 2. Those of skill in the art appreciate that other proteins are involved in silk color, texture, smoothness, uniformity, length, strength, weight and the ability to absorb dyes, but the genetic loci have not been determined.

Protease inhibitors in A/MSG may play an important role in protecting the fibroin proteins in the silk gland lumen against digestion by proteases, such as antennal esterase and serine protease, which are expressed in the A/MSG. In another embodiment, the genetically modified silkworm may comprise an edited chromosomal sequence encoding protease inhibitor in A/MSG. A modified protease inhibitor coding region may give rise to a silk protein with different physical properties. In one embodiment, the genetically modified silkworm comprising a modified protease inhibitor chromosomal region may have a phenotype producing silk without high percentage of sericin yet still intact in shape and other physical properties.

In still another embodiment, the genetically modified silkworm may comprise an edited chromosomal sequence encoding Fibroin, Sericin, solute carrier, protease inhibitor or combinations thereof. The edited chromosomal sequence may comprise at least one modification such that an altered version of Fibroin, Sericin or other silk fiber formation related proteins is produced. The chromosomal sequence may be modified to contain at least one nucleotide change such that at the expressed protein comprises at least one amino acid change as detailed above. Alternatively, the edited chromosomal sequence may comprise a mutation such that the sequence is inactivated and no protein is made or a defective protein is made. As detailed above, the mutation may comprise a deletion, an insertion, or a point mutation. The genetically modified silkworm comprising an edited FibH, ser1 and/or protease inhibitor chromosomal sequence may have a different fiber color, texture, weight than a silkworm in which said chromosomal region(s) is not edited.

Silk is naturally hypoallergenic. However, several people experience silk allergies for a wide variety of causes. Often, the allergies are traced to the diet of the silk worm, such as mulberry or oak leaves, which influence the protein chains found in the silk strands produced by the silkworm. Silk allergies can cause asthma or allergic rhinitis. In one embodiment, the genetically modified silkworm may comprise an edited chromosomal sequence encoding alpha and beta glucosidases, glycoside hydrolase, and glucose transporters are all involved in glucose hydrolysis and transport in the digestion of mulberry leaves, the sole food source for the silkworm. These proteins are expressed in the midgut of the silkworm and are related to functions such as nutrient digestion, transportation, and absorption. In another embodiment, the genetically modified silkworm may comprise an edited chromosomal sequence encoding the lipase protein family, antennal esterases, carboxylesterase, and scavenger receptor SR-B1, which are associated mainly with lipid metabolism in the midgut, such as the hydrolysis of triglycerides, degradation of odorant acetate compounds, and the binding of modified low-density lipoproteins. The genetically modified silkworm comprising the edited chromosomal sequence described above generally will not contain allergen, which causes silk allergic reactions for silk manufacturing workers and consumers.

The midgut also represents the first line of resistance and immune response of the silkworm. In one embodiment, the genetically modified silkworm may comprise an edited chromosomal sequence encoding aminopeptidases which bind various classes of the Cry toxins. For example, BtR175, a cadherin-like protein expressed in the silkworm, functions as a Cry toxin receptor in signal transduction. In another embodiment, the genetically modified silkworm may comprise an edited chromosomal sequence of 17 members of the cytochrome P450 gene family in the midgut, which include CYP4, CYP6 and CYP9. The cytochrome P450 gene family in the midgut is involved in metabolism of plant toxins and insecticides.

Another midgut-specific gene encodes peptidoglycan recognition protein, which can bind strongly to the cell wall peptidoglycans of Gram-positive bacteria and trigger the immune response. Furthermore, two lymphocyte receptor genes expressed specifically in the midgut encode binding proteins that function in the recognition of pathogens. In another group of embodiments, the genetically modified silkworm may comprise an edited chromosomal sequence in peptidoglycan recognition protein or lymphocyte binding protein, wherein the chromosomal sequence is up-regulated such that the silkworm is more disease resistant. With suitable mutations discussed above, the genetically modified silkworm generally will have better immune system, is disease- and pathogen-free, and is less susceptible to plant toxins and insecticides in its food source.

Similar to silkworm fiber, spider silk is another naturally made fiber which is three times tougher than Kevlar®, a material used in the army's current ballistic protective vest. Spider silk's superior ability to elongate allows it to absorb more energy in breaking and slow down of a projectile more effectively. However, the strong, pliable silk that spiders produce is not practical to harvest. The gene to make spider silk from the spider N. clavipes has been cloned, and there are also synthetic genes to mimic the spider dragline silk. In one embodiment, the genetically modified silkworm may comprise an edited chromosomal sequence that comprises an integrated sequence, such as flagelliform gene, coding spider silk. The genetically modified silkworm will enable a systematic and high-volume production of spider silk for the need of new material with unique properties.

The present disclosure also encompasses a genetically modified silkworm comprising any combination of the above described chromosomal alterations. For example, the genetically modified silkworm may comprise a modified FibH and/or FibL chromosomal sequence, a modified serf chromosomal sequence, and/or a modified BtR175, and/or CYP4 chromosomal sequence, and/or integrated sequence from other species or organisms.

The silkworms and cells disclosed herein may have several applications. In one embodiment, the genetically modified silkworm comprising at least one edited chromosomal sequence may exhibit a phenotype desired by humans. For example, modification of the chromosomal sequence encoding Fibroin may result in silk fiber that carries unique color, texture, weight or strength. In other embodiments, the silkworm comprising at least one edited chromosomal sequence may be used as a model to study the genetics of silk composition, production, and/or transportation. Additionally, a silkworm comprising at least one disrupted chromosomal sequence may be used as a model to study a disease or condition that affects humans or other animals. Non-limiting examples of suitable diseases or conditions include mulberry allergy. Additionally, the disclosed silkworm cells and lysates of said cells may be used for similar research purposes.

### III. Animal Comprising a Chromosomal Edit

One aspect of the present disclosure provides a genetically modified animal in which at least one chromosomal sequence is edited. Suitable chromosomal edits may include, but are not limited to, the types of edits detailed in section I(f) above.

The term “animal,” as used herein, refers to a non-human animal. The animal may be an embryo, a juvenile, or an adult. Suitable animals may include vertebrates such as mammals, birds, reptiles, amphibians, and fish. Examples of suitable mammals may include, without limit, rodents, companion animals, livestock, and primates. Non-limiting examples of rodents include mice, rats, hamsters, gerbils, and guinea pigs. Suitable companion animals may include but are not limited to cats, dogs, rabbits, hedgehogs, and ferrets. Non-limiting examples of livestock include horses, goats, sheep, swine, cattle, llamas, and alpacas. Suitable primates may include, but are not limited to, new-world monkeys, old world monkeys, and apes, such as for example, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples insects include a silkworm, Drosophila and a mosquito.

In one embodiment, an exemplary animal is a rat. Non-limiting examples of suitable rat strains include Dahl Salt-Sensitive, Fischer 344, Lewis, Long Evans Hooded, Sprague-Dawley, and Wistar.

In each of the foregoing iterations of suitable animals for the invention, the animal does not include exogenously introduced, randomly integrated transposon sequences.

An animal of the invention may include a genomic edit in a gene listed in section II above, or in Tables A, B, C and D above. In an additional embodiment, an animal of the invention may include a genomic edit as described in the Examples.

In an exemplary embodiment, a method of the invention may be used to develop an animal, derived from the embryo, which comprises a chromosomal edit on at least one chromosome in every cell of the animal. In some embodiments, a method may be used to develop an animal that comprises a chromosomal edit on two chromosomes in every cell of the animal. In some embodiments, a chromosomal edit is on at least one autosome. In other embodiments, a chromosomal edit is on at least one sex chromosome.

Two animals of the invention may be crossbred to create an animal homozygous for a chromosomal edit. Alternatively, an animal may be crossbred to combine a chromosomal edit with other genetic backgrounds. By way of non-limiting example, other genetic backgrounds include genetic backgrounds with another chromosomal edit, genetic backgrounds with non-targeted integrations, genetic backgrounds with deletion mutations, and wild-type genetic backgrounds. In one embodiment, for example, an animal A may comprise a first chromosomal edit, and an animal B may comprise a second chromosomal edit. An F1 generation that comprises both the first and second chromosomal edits may be obtained by breeding A with B. This or a similar breeding scheme is one method for the combination of more than one chromosomal edit in the same animal.

In certain embodiments, an animal comprising a chromosomal edit may be used to create primary cell lines, as detailed in section IV. below. A resulting cell line would comprise the chromosomal edit originally introduced into the embryo. An animal of the invention may be used in any of the applications detailed in section II. above.

### IV. Genetically Modified Cell

Still yet another aspect of the invention encompasses a cell created by a method of the invention, i.e. a cell that comprises at least one chromosomal edit. Suitable edits may include, but are not limited to, the types of edits detailed in section I(f) above.

The type of cell comprising at least one chromosomally edit can and will vary. In general, the cell will be a eukaryotic cell. In some instances, the cell may be a primary cell, a cultured cell, or immortal cell line cell. Suitable cells may include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CV1-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In an exemplary embodiment, a cell of the invention is an embryo. An embryo may be a one cell embryo, or a more than one cell embryo. Suitable embryos may be derived from several different vertebrate species, including mammalian, bird, reptile, amphibian, and fish species. Generally speaking, a suitable embryo is an embryo that may be collected, injected, and cultured to allow the expression of a zinc finger nuclease. In some embodiments, suitable embryos may include embryos from rodents, companion animals, livestock animals, and primates. Non-limiting examples of rodents include mice, rats, hamsters, gerbils, and guinea pigs. Non-limiting examples of companion animals include cats, dogs, rabbits, hedgehogs, and ferrets. Non-limiting examples of livestock include horses, goats, sheep, swine, llamas, alpacas, and cattle. Non-limiting examples of primates include capuchin monkeys, chimpanzees, lemurs, macaques, marmosets, tamarins, spider monkeys, squirrel monkeys, and vervet monkeys. In other embodiments, suitable embryos may include embryos from fish, reptiles, amphibians, or birds. Alternatively, suitable embryos may be insect embryos, for instance, a Drosophila embryo, a mosquito embryo, or a silkworm embryo. A skilled artisan will appreciate that methods for the collection, injection, and culturing of embryos are known in the art and can and will vary depending on the species of embryo. Routine optimization may be used, in all cases, to determine the best techniques for a particular species of embryo.

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

Additionally, a cell of the invention may be modified to include a tag or reporter gene. Reporter genes include those encoding selectable markers such as cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor or exchange polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate-FPIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that may changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

The following examples illustrate various iterations of the invention.

### Example 1

### Construction of Restriction Fragment Length Polymorphism (RFLP) Donor Nucleic Acid for Targeted Integration into the PXR Nucleic Acid Region of the Rat Genome

There are two possible DNA repair outcomes after a targeted, ZFN-induced double-stranded break (FIG. 1). The break may be repaired by non-homologous end joining (NHEJ), leading to mutations containing base deletions or additions or, in the presence of a donor DNA, the donor DNA can be used as a template to repair the double stranded break by homologous recombination (HR). If the donor DNA encodes specific sequence changes, these deliberate mutations will be incorporated into the genome of the organism at the target site.

To test targeted integration in the rat genome using pronuclear injection, constructs were designed and prepared for targeted integration into the PXR gene region of the rat genome. Constructs were assembled to introduce either a NotI or PmeI restriction fragment length polymorphism (RFLP) site into the PXR gene region (FIG. 2). The constructs were designed with either 200, 800 or 2000 base pairs of sequence homology to the PXR gene target site flanking the RFLP sites to be introduced. The three sizes of regions of homology were used to determine the size of homology required for efficient targeting and homologous recombination.

The clones were assembled using PCR amplification to introduce convenient restriction sites for cloning, and the RFLP site at the extremities of the PXR homology regions (FIG. 1). PCR primers used for amplifying the PXR region of homology are described in Table 1. Accuprime HF DNA polymerase was used for PCR reaction amplification. A 30s extension was used for the 200 bp fragments, a 1.5 min extension was used for the 800 bp fragments, and a 4 min extension was used for the 2 Kbp fragments. PCR fragments were then digested with the appropriate restriction enzymes and cloned into pBluescript using three-way ligation to produce six plasmids listed in Table 2.

TABLE 1
Primer sequences

| | | SEQ. |
|---|---|---|
| Name | Sequence | ID NO. |
| PXR 200 bp F KpnI | 5′-aaaaggtacctctgtgtttttccgttctagtccag | 1 |
| PXR 200 bp R SacII | 5′-aaaaccgcggctgaagtatacgtggctctcttgga | 2 |
| PXR target F NotI | 5′-gtgtagcggccgcgacaaggccaatggctatcac | 3 |
| PXR target F PmeI | 5′-gtgtagtttaaacgacaaggccaatggctatcac | 4 |
| PXR target R NotI | 5′-ttgtcgcggccgctacacggcagatttgaagacctc | 5 |
| PXR target R PmeI | 5′-ttgtcgtttaaactacacggcagatttgaagacctc | 6 |
| PXR 800 bp F KpnI | 5′-aaaaggtacctcagactggtccagattttagamaagggg | 7 |
| PXR 800 bp R SacII | 5′-aaaaccgcggataaatctactggttcgccaagctag | 8 |
| PXR 2 Kb F KpnI | 5′-aaaaggtaccgaggtagtaggaaatgcacttc | 9 |
| PXR 2 Kb R SacII | 5′-aaaaccgcgggaagagaattattgctgacagtc | 10 |
| PXR 50 bp F | 5′-gagcctatcaacgtagatgagg | 11 |
| PXR 50 bp R | 5′-cttacatccttcacaggtcatgac | 12 |

TABLE 2
Plasmids constructed

| RFLP introduced | Length of region of homology |
|---|---|
| NotI | 200 bp |
| NotI | 800 bp |
| NotI | 2 Kbp |
| PmeI | 200 bp |
| PmeI | 800 bp |
| PmeI | 2 Kbp |

### Example 2

### Construction of Restriction Fragment Length Polymorphism (RFLP) Donor Nucleic Acid for Targeted Integration into the rRosa26 Nucleic Acid Region of the Rat Genome

Plasmids were also constructed to target integration of NotI and PmeI RFLP sites into the rRosa26 nucleic acid region of the rat genome. Design and construction of the plasmids was as described in Example 1 above. The PCR primer pairs used for amplifying the rRosa26 region of homology are described in Table 3.

TABLE 3
Primer sequences

| Name | Sequence | SEQ. ID NO. |
|---|---|---|
| rRosa26 200 bp F KpnI | aaaaggtaccgggagtggatgaaggagttg | 13 |
| rRosa26 200 bp R SacII | aaaaccgcggcggatcacaagcaataat | 14 |
| rRose26 target F NotI | cttcgcggccgcgatctgcaactggagtctttc | 15 |
| rRosa26 target F PmeI | cttcgtttaaacgatctgcaactggagtctttc | 16 |
| rRosa26 target F NotI | gatcgcggccgcgaagaagggggaagggaatc | 17 |
| rRosa26 target R PmeI | gatcgtttaaacgaagaagggggaagggaatc | 18 |
| rRosa26 800 bp F KpnI | aaaaggtaccgcgtgtgaaaacacaaatgg | 19 |
| rRosa26 800 bp R SacII | aaaaccgcggaaggaaagaggcattcatgg | 20 |
| rRosa26 2 Kb F KpnI | aaaaggtaccattatggaggggaggactgg | 21 |
| rRosa26 2 Kb R SacII | aaaaccgcggacatgtggcaaacaggaga | 22 |
| rRosa26 50 bp F | tgtcttctgaggaccgccc | 23 |
| rRosa26 50 bp R | ctgcccagaagactcccgc | 24 |

### Example 3

### Construction of Restriction Fragment Length Polymorphism (RFLP) Donor Nucleic Acid for Targeted Integration into the Mdr1a Nucleic Acid Region of the Mouse or Rat Genome

Plasmids were constructed to target integration of NotI and PmeI RFLP sites into the mMdr1a nucleic acid region of the mouse genome or the rMdr1a nucleic acid region of the rat genome. Design and construction of the plasmids was as described in Example 1 above. The PCR primer pairs used for amplifying the Mdr1a region of homology are described in Table 4. “m” stands for mouse and “r” stands for rat.

TABLE 4

| Name | Sequence | SEQ. ID NO. |
|---|---|---|
| mMdr1a 200 bp F KpnI | aaaaggraccaacaacactaggctcaggag | 25 |
| mMdr1a 200 bp R SacII | aaaaccgcggcacatggctaagcacagcatg | 26 |
| mMdr1a target F NotI | cctgcggccgcggactgtcagctggtatttg | 27 |
| mMdr1a target F PmeI | cctgtttaaacggactgtcagctggtatttg | 28 |
| mMdr1a target R NotI | gtccgcggccgcagggctgatggccaaaatc | 29 |
| mMdr1a target R PmeI | gtccgtttaaacagggctgatggccaaaatc | 30 |
| mMdr1a 800 bp F KpnI | aaaaggtaccatgctgtgaagcagatacc | 31 |
| mMdr1a 800 bp R SacII | aaaaccgcggctgaaaactgaatgagacatttgc | 32 |
| mMdr1a 2KB F KpnI | aaaaggtaccgtaatgttccaattgcatcttcc | 33 |
| mMdr1a 2KB R SacII | aaaaccgcggctctcagttctctgctgttg | 34 |
| mMdr1a 50 bp F | gatttacccgtggctggaag | 35 |
| mMdr1a 50 bp R | ctggactcatggacttcacc | 36 |
| rMdr1a 200 bp F KpnI | aaaaggtacctggctcaggagaaaaattgtg | 37 |
| rMdr1a 200 bp R SacII | aaaaccgcggcacggctaaagacagcatga | 38 |
| rMdr1a target F NotI | ccctgcggccgcggactgtcagctggtatttg | 39 |
| rMdr1a target F PmeI | ccctgtttaaacggactgtcagctggtatttg | 40 |
| rMdr1a target R NotI | gtccgcggccgcagggctgatggccaaaatc | 41 |
| rMdr1a target R PmeI | gtccgtttaaacagggctgatggccaaaatc | 42 |
| rMdr1a 800 bp F KpnI | aaaaggtaccggagataggctggtttgacg | 43 |
| rMdr1a 700 bp R SacII | aaaaccgcggatggtggtagttcggatgg | 44 |
| rMdr1a 2 Kb F KpnI | aaaaggtaccaggttgttcttggagatgtgc | 45 |
| rMdr1a 2 Kb T SacII | aaaaccgcggtcctcttggctggtgagttt | 46 |
| rMdr1a 50 bp F | gatttactcgcggctggaag | 47 |
| rMdr1a 50 bp R | ctggactcacgggcttcac | 48 |

### Example 4

### Construction of GFP Expression Integration Cassette

To test targeted integration of nucleic acid fragments larger than RFLPs, constructs were designed and prepared for targeted integration of a GFP expression cassette into the PXR and rRosa26 nucleic acid genomic regions of the rat and the mMdr1a nucleic acid genomic regions of the mouse. Briefly, a GFP expression cassette containing the human PGK promoter, the GFP open reading frame, and a polyadenylation signal was amplified using PCR to introduce NotI restriction sites at the extremities (FIG. 3) using the following primers: PGKGFP-F NotI (5′-aaagcggccgcttggggttgcgccttttcc) (SEQ ID NO:49) and PGKGFP-R NotI (5′-aaaagcggccgccatagagcccaccgcatc) (SEQ ID NO:50). The PCR fragment was then cloned into the NotI-containing plasmids constructed in Examples 1-3.

### Example 5

### Preparation of Zinc Finger mRNAs for Targeted Integration

A pair of zinc finger nucleases were designed for each targeted integration site and cloned as described on the Sigma web site. For more information, see Science (2009) 325:433, herein incorporated by reference. ZFN expressing mRNAs were then produced in vitro by first digesting 20 μg of each maxiprepped ZFN expression plasmid DNA in 100 μl reactions containing 10 μl buffer 2 (NEB, #B7002S), 10 μl 10×BSA (diluted from 100×BSA, NEB, #B9001S), 8 μl XbaI (NEB, #R0145S), at 37° C. for 2 h. The reactions were extracted with 100 μl of phenol/chloroform (Sigma, P2069), centrifuged at over 20,000×g for 10 min. The aqueous supernatant was precipitated with 10 μl 3M NaOAc (Sigma, S7899) and 250 μl 100% ethanol and centrifuged at top speed for 25 min at room temperature. The resulting pellet was washed by adding 300 μl 70% ethanol filtered through a 0.02 μM filter. The pellet was air dried and resuspended in 20 μl of 0.02 μM filtered 0.1×TE.

The purified digested DNA was then used to produce ZFN transcripts using in vitro transcription with MessageMax T7 Capped Message Transcription Kit (#MMA60710) from Epicentre Biotechnologies as described. In short, kit components are prewarmed to room temperature, and reaction components for a 20 μl reaction were combined at room temperature in the following order: 5 μl of 0.02 um filtered RNase-free water, 1 μl prepared template, 2 μl lox transcription buffer, 8 μl 2-way Cap/NTP premix, 2 μl 100 mM DTT and 2 μl MessageMax T7 Enzyme Solution. The reactions were then incubated in a 37° C. incubator for 30 min.

The capped RNA was then tailed with polyA using the A-Plus Poly (A) Polymerase tailing kit (Epicentre, #PAP5 104H) as described. Reaction components were combined at room temperature in the following given order: 55.5 μl 0.02 um filtered RNase-free water, 10 μl 10×A-Plus Reaction Buffer, 10 ul 10 mM ATP, 2.5 μl ScriptGuard RNase Inhibitor (40 unit/pi), 20 μl In vitro transcription capping reaction, 2 μl A-plus poly A polymerase. The reaction was then incubated at 37° C. for 30 min. The resulting capped polyA-tailed mRNA was purified by precipitation with an equal volume of 5M NH₄Oac twice. The mRNA pellet was then air dried, and resuspended in 30 μl of filtered injection buffer (1 mM Tris, pH7.4, 0.25 mM EDTA), and RNA concentration was measured using a Nanodrop spectrophotometer.

### Example 6

### Targeted Integration into Embryos

To integrate nucleic acids into the rat or mouse genome, zinc finger nuclease mRNA was mixed with the maxiprepped target DNA filtered with 0.02 um filters. The nucleic acid mixture consisted of one part ZFN mRNAs to one part donor DNA. The nucleic acid mixture was then microinjected into the pronucleus of a one-celled embryo using known methods. The injected embryos were either incubated in vitro, or transferred to pseudo moms. The resulting embryos/fetus, or the toe/tail clip of live born animals were harvested for DNA extraction and analysis.

To extract DNA, tissue was lysed in 100 μl Epicentre's QuickExtract at 50° C. for 30 min, followed by incubation at 65° C. for 10 min, and 98° C. for 3 min. To determine if targeted integration occurred, PCR was used to amplify the target region using appropriate primers. For experiments where RFLP was integrated into the genome of the animal, the PCR products were digested with the introduced RFLP enzyme to detect integration (FIG. 4A). In addition, a Cel-I endonuclease assay using wild type PCR fragments and PCR fragments derived from injected embryos was used to demonstrate that ZFN mRNA was functional in the embryos by detecting NHEJ, which is independent of targeted integration. For experiments where GFP was integrated into the genome of the animal, a shift in size of the PCR fragment is indicative of the integration (FIG. 4B). Alternatively, amplification of the integration junction, where one primer lands only on the GFP cassette was used to assess integration of the donor nucleic acid.

### Example 7

### Testing of DNA Extraction and PCR Amplification of the mMdr1a Target Site in the Mouse Genome

PCR conditions to amplify target nucleic acid extracted from tissue were tested using embryos with 1-64 cells extracted as described in Example 6. A 900 bp fragment containing the mouse mMdr1a target region was amplified using 36 amplification cycles with 4 min extension at 60° C. in reactions containing up to 5 μl Epicentre's QuickExtract solution in 50 μl reactions (FIG. 5). These results show that QuickExtract does not interfere with PCR amplification, and that DNA can be amplified from sample extracted from only 1-10 cells. To enhance sensitivity, the number of PCR cycles may be increased, or nested PCR reactions may be performed.

### Example 8

### Integration of NotI Donor RFLP into the Rat PXR Genomic Region—Experiment A

A donor plasmid (with an 800 bp arm) for integrating a NotI RFLP site into the PXR region of the rat genome was injected into rat embryos with ZFN mRNAs as described above. PCR, followed by NotI restriction enzyme analysis and Cel-I endonuclease analysis were performed using DNA extracted from a number of embryos. PCR amplification was successful with a number of embryos (FIG. 6A), and Cel-I endonuclease analysis revealed that most of the fragments had nucleic acid sequence changes at the desired target (FIG. 6B).

### Example 9

### Integration of NotI Donor RFLP into the Mouse mMdr1a Genomic Region—Experiment B

The targeted integration of the NotI RFLP into the mouse mMdr1a region was repeated as described in Example 8. The mMdr1a region was amplified using PCR and digested with NotI. PCR amplification was successful with a number of embryos (FIG. 7), and digestion with NotI revealed that a number of embryos comprised the integrated RFLP site (see e.g. lanes 13, 17, 19, 20 and 23). In all, targeted integration in 7 out of the 32 embryos for which data was generated.

These results were confirmed by repeating the NotI digestion reaction after further cleaning the PCR reaction product (FIG. 8).

### Example 10

### Testing DNA Extraction and PCR Amplification of the PXR Target Site in the Rat Genome

PCR amplification of the PXR region from blastocysts was tested to determine the level of sensitivity. The PCR reaction contained 5 μl template, 5 μl PCR buffer, 5 μl of each primer, 0.5 μl of Taq polymerase enzyme, and 33.5 μl water for a 50 μl reaction. The template consisted of undiluted DNA extracted from rat blastocysts or DNA diluted at a ratio of 1:2, 1:6, 1:10, and 1:30 (FIG. 9).

### Example 11

### Integration of NotI Donor RFLP into the Rat PXR Genomic Region

A donor plasmid (with 800 bp homology arms) for integrating a NotI RFLP site into the PXR region of the rat genome was injected into rat embryos with ZFN mRNAs as described above. A total of 123 embryos were injected, and 106 survived. Decreasing concentrations of nucleic acids were injected to test for toxicity. Of the 51 embryos injected with 5 ng of nucleic acids, 17 survived and divided to two cell embryos on day two. Of the 23 embryos injected with 2 ng of nucleic acids, 14 survived and divided to two cell embryos on day two. Of the 29 embryos injected with 10 ng of nucleic acids, 12 survived and divided to two cell embryos on day two. Of the ten uninjected control embryos, all survived and divided to two cell embryos on day two.

PCR amplification of the PXR region, followed by NotI and Cel-I endonuclease analysis were performed using DNA extracted from a number of embryos. PCR amplification was successful with a number of embryos, and NotI and Cel-I endonuclease analysis revealed that 18 out of 47 embryos had nucleic acid sequence changes at the desired target (FIG. 10).

### Example 12

### Targeted Integration of RFLP into the mMdr1a Target Region of the Mouse Genome in Fetus

A donor plasmid (with 800 bp homology arms) for introducing NotI into the mMdr1a region of the mouse genome was injected into mouse embryos with ZFN mRNAs as described above. One out of four well-developed fetuses at 12.5 dpc were positive for the NotI site. All four deciduas were negative. (FIG. 11).

### Example 13

### Targeted Integration of GFP into the mMdr1a Locus of a Fetus

A donor plasmid (with 800 bp homology arms) for introducing GFP cassette into the mMdr1a region of the mouse genome was injected into mouse embryos with ZFN mRNAs as described above. Two out of forty fetuses at 12.5 dpc were positive for the GFP cassette (FIG. 12).

### Example 14

### Targeted Integration of RFLP into the PXR Target Region of the Rat Genome in a Fetus

A donor plasmid (with 800 bp homology arms) for introducing NotI into the PXR region of the rat genome was injected into mouse embryos with ZFN mRNAs as described above. One out of eight fetuses at 13 dpc were positive for the NotI site (FIG. 13).

### Example 15

### Genome Editing of SMAD4 in Cat Cells

Zinc finger nuclease (ZFN)-mediated genome editing was tested in cat cells using a ZFN that binds to the human SMAD4 chromosomal sequence because the DNA binding sites in cat and human are identical. The amino acid sequence and corresponding DNA binding site of SMAD4 ZFN pair (19160/19159) are presented in TABLE 5. Capped, polyadenylated mRNA encoding SMAD4 ZFNs (19160/19159) were produced using known molecular biology techniques. The mRNA was transfected into human K562, feline AKD (lung), and feline CRFK (kidney) cells. Control cells were injected with mRNA encoding GFP.

TABLE 5
SMAD4 ZFNs

| | | SEQ | DNA binding site | SEQ |
|---|---|---|---|---|
| | | ID | (Contact sites in | ID |
| Name | ZFN protein sequence | NO: | uppercase; 5′-3′)) | NO: |
| 19159 | VPAAMAERPFQCRICMRNFSRSDNLAR | 53 | ctGCTGTCCTGGCTG | 55 |
| | HIRTHTGEKPFACDICGRKFAQSSDLRR | | AGgccctgatgct | |
| | HTKIHTGGQRPFQCRICMRNFSRSDTL | | | |
| | SQHIRTHTGEKPFACDICGRKFADRSAR | | | |
| | TRHTKIHTGEKPFQCRICMRKFAQSSDL | | | |
| | RRHTKIHLRGS | | | |
| 19160 | VPAAMAERPFQCRICMRNFSERGTLAR | 54 | gaATGGATtTACTGG | 56 |
| | HIRTHTGEKPFACDICGRKFAQSADRTK | | TCAGCCagctact | |
| | HTKIHTGGQRPFQCRICMRNFSRSDHL | | | |
| | STHIRTHTGEKPFACDICGRKFADNANR | | | |
| | TKHTKIHTGGGGSQKPFQCRICMRNFS | | | |
| | QSSNLARHIRTHTGEKPFACDICGRKFA | | | |
| | RSDALTQHTKIHLRGS | | | |

The frequency of ZFN-induced double strand chromosomal breaks was determined using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ. As shown in FIG. 14, the SMAD4 ZFNs (19160/19159) cleaved the SMAD4 locus in human and cat cells.

### Example 16

### Genome Editing of SMAD4 in Cat Embryos

Cat embryos were harvested using standard procedures and injected with capped, polyadenylated mRNA encoding SMAD4 ZFNs (19160/19159) using techniques substantially similar to those described by Geurts et al. Science (2009) 325:433, which is incorporated by reference herein in its entirety. The cat embryos were at the 2-4 cell stage when microinjected. Control embryos were injected with 0.1 mM EDTA. The frequency of cutting was estimated using the Cel-1 assay as described in Example 15. As illustrated in FIG. 15, the cutting efficiency was estimated to be about 6-9%.

TABLE 6 presents the development of the embryos following microinjection. About 19% ( 3/16) of the embryos injected with a small volume of SMAD4 ZFN mRNA developed to the blastula stage, and 50% ( 8/16) of the control embryos injected with EDTA developed to the blastula stage.

TABLE 6
Embryo development

| | | | No. | | | Day 5 | | |
|---|---|---|---|---|---|---|---|---|
| | | | oocytes | | | Degenerated/ | | Day 7/8 |
| | | No. | IVF | Day 2 | | Non- | Morula/ | Blastocysts/ |
| | No. | oocytes | or | | Cleaved | further- | cleaved | cleaved |
| Treatment | replicates | collected | injected | Degenerated | (2-4 cells) | cleavage | embryos | embryos |
| IVF | N = 2 | 58 | 54 | 1/54 | 34/54 | 0/34 | 34/34 | 33-34 |
| (control) | | | | (1.8%) | (62.9%) | (0%) | (100%) | (97%) |
| 0.1 mM | N = 1 | 18 | 16 | 0/16 | 15/16 | 1/15 | 14/15 | 8/15 |
| EDTA | | | | (0%) | (93.7%) | (6.6%) | (93.3%) | (53.3%) |
| ZFN- | Higher | 44 | 36 | 2/36 | 34/36 | 29/31 | 2/31 | 0/31 |
| SMAD-4 | volume | | | (5.5%) | (86.1%) | (93.5%) | (6.4%) | (0%) |
| (10 ng) | N = 1 | | | | | | | |
| | Smaller | 20 | 16 | 3/16 | 5/16 | 2/5 | 3/5 | 3/5 |
| | volume | | | (18.7%) | (31.2%) | (40.0%) | (60%) | (60%) |
| | N + 1 | | | | | | | |

### Example 17

### Genome Editing of Fel d1 in Cat Cells

ZFNs were designed to target different regions of the Fel d1 chromosomal sequence in cat (see Geurts et al. (2009) supra). The ZFNs targeted chain 1-exon 1, chain 1-exon 2, or chain 2-exon 2 of Fel dl. The amino acid sequence and DNA binding site of each ZFN are shown in TABLE 7.

TABLE 7
Fel d1 ZFNs

| | | SEQ | DNA binding site | SEQ |
|---|---|---|---|---|
| | | ID | (Contact sites in | ID |
| Name | ZFN protein sequence | NO: | uppercase) | NO: |
| 17 | VPAAMAERPFQCRICMRNFSRSDH | 57 | acAGTAGGGCAGGG | 63 |
| (ch1,ex1) | LSTHIRTHTGEKPFACDICGRKFAR | | TGGgagggctgcgt | |
| | SAHLSRHTKIHTGSQKPFQCRICMR | | | |
| | NFSQSGSLTRHIRTHTGEKPFACDI | | | |
| | CGRKFARSDHLTQHTKIHTGEKPFQ | | | |
| | CRICMRKFALKQHLNEHTKIHLRGS | | | |
| 18 | VPAAMAERPFQCRICMRNFSRSDN | 58 | ggCCACAGCAGGTA | 64 |
| (ch1,ex1) | LSAHIRTHTGEKPFACDICGRKFAQ | | TAAAAGggttccag | |
| | SANRIKHTKIHTGSQKPFQCRICMR | | | |
| | NFSQSGALARHIRTHTGEKPFACDI | | | |
| | CGRKFARSDNLREHTKIHTGSQKPF | | | |
| | QCRICMRNFSRSDHLSEHIRTHTGE | | | |
| | KPFACDICGRKFAQSATRKKHTKIH | | | |
| | LRGS | | | |
| 7 | VPAAMAERPFQCRICMRNFSQSGH | 59 | tcGTCGGGggTTCCC | 65 |
| (ch1,ex2) | LARHIRTHTGEKPFACDICGRKFAQ | | GTCAGGAataggt | |
| | SADRTKHTKIHTGSQKPFQCRICMR | | | |
| | NFSRSDTLSEHIRTHTGEKPFACDIC | | | |
| | GRKFANRRGRWSHTKIHTPNPHRR | | | |
| | TDPSHKPFQCRICMRNFSRSDHLS | | | |
| | RHIRTHTGEKPFACDICGRKFADPS | | | |
| | YLPRHTKIHLRGS | | | |
| 9 | VPAAMAERPFQCRICMRNFSRSDS | 60 | atGTTGAGCAAGTGg | 66 |
| (ch1,ex2) | LSVHIRTHTGEKPFACDICGRKFAQ | | cacaatacaatgc | |
| | NANRKTHTKIHTGSQKPFQCRICMR | | | |
| | NFSRSANLARHIRTHTGEKPFACDI | | | |
| | CGRKFATSGSLTRHTKIHLRGS | | | |
| 12 | VPAAMAERPFQCRICMRNFSRSDT | 61 | aaGAGTCCGTTcTCC | 67 |
| (ch2,ex2) | LSAHIRTHTGEKPFACDICGRKFAD | | ACGtagcaatcct | |
| | KRTRTTHTKIHTHPRAPIPKPFQCRI | | | |
| | CMRNFSTSGSLSRHIRTHTGEKPFA | | | |
| | CDICGRKFADSSDRKKHTKIHTGEK | | | |
| | PFQCRICMRKFARSDNLTRHTKIHL | | | |
| | RGS | | | |
| 13 | VPAAMAERPFQCRICMRNFSRSDT | 62 | ccAGGGTCtTGGATG | 68 |
| (ch2,ex2) | LSAHIRTHTGEKPFACDICGRKFAD | | GACTAGtcatggt | |
| | KRTRTTHTKIHTHPRAPIPKPFQCRI | | | |
| | CMRNFSTSGSLSRHIRTHTGEKPFA | | | |
| | CDICGRKFADSSDRKKHTKIHTGEK | | | |
| | PFQCRICMRKFARSDNLTRHTKIHL | | | |
| | RGS | | | |

Feline AKD cells were transfected with mRNA encoding Fel d1 ZFNs (17/18), which target exon 1 of chain 1; Fel d1 ZFNs (7/9), which target exon 2 of chain 1, or Fel d1 ZFNs (12/13), which target exon 2 of chain 2. The efficiency of ZFN-mediated cutting was estimated using the Cel-1 assay as described above. The cutting efficiency of the 17/18 Fel d1 ZFN pair was estimated to be about 17% (see FIG. 16). The 7/9 Fel d1 ZFN pair cleaved chain 1, exon 2 with an efficiency of about 16% (see FIG. 17). FIG. 18 illustrates that chain 2, exon 2 was cleaved by the 12/13 Fel d1 ZFN pair.

### Example 18

### Genome Editing of Fel d1 in Cat Embryos

To facilitate inactivation of the Fel d1 locus, cat embryos were treated with two pairs of Fel d1 ZFNs. One pair (17/18) targeted chain 1-exon 1 and the other pair (12/13) targeted chain 2-exon 2. Because of the genomic organization of Fel d1 locus, the coding region for chain 2 (which is transcribed from the “lower” strand) is located about 4000 bp upstream of the coding region for chain 1 (which is transcribed from the “upper” strand). Thus, it was hypothesized that editing events are two separate locations may mediate a large deletion from the Fel d1 locus. Cat embryos were co-injected with capped, polyadenylated mRNAs encoding the pairs of ZFNs essentially as described above in Example 16. TABLE 8 presents the development of the embryos following microinjection. Embryos injected with the higher concentration Fel dl ZFNs had a higher survival rate than those injected with the lower concentration.

TABLE 8
Embryo development.

| No. of | | | No. of | Day 2 | | Day 5 | | Day 7/8 |
|---|---|---|---|---|---|---|---|---|
| oocytes | | | fertilized | | Cleaved | Degen. | Morula/ | Blast./ |
| (n = | | No. | oocytes | | (2-4 | No addl | cleav. | cleav. |
| cats) | Treatment | repl. | injected | Degen. | cells) | cleav. | embryo | embryo |
| 100 | IVF | | 5 | | 5 | | 5 | 4 |
| (n = 4) | (Cont) | | | | | | | (80%) |
| | EDTA | | | | | | | |
| | (Cont) | | | | | | | |
| | Feld1 | 2 | 31 | 4 | 18 | 3 | 15 | 4 |
| | 40 ng/ | | | | | | | (22%) |
| | uL | | | | | | | |
| | Feld1 | 1 | 27 | 1 | 14 | 3 | 11 | 1 |
| | 20 ng/ | | | | | | | (7.1%) |
| | uL | | | | | | | |

On day 8, the control and experimental embryos were harvested for analysis. Control blastocysts contained about 150-300 cells, experimental blastocysts contained about 70-100 cells, and experimental morula contained about 16-30 cells. DNA of individual embryos was extracted using standard procedures, and subjected to Cel-1 analysis (see FIG. 19). Samples in lanes 1, 3, and 7 displayed the expected Cel-1 digestion products. Sequence analysis revealed that extra bands in other lanes (including the control lane, 6) were due to nearby SNPs.

To further analyze the edited Fel d1 loci, the targeted region was PCR amplified and sequenced using standard methods. Sequence analysis confirmed that sample #5 had a 4541 bp deletion between the coding regions for chain 2 and chain 1 (see FIG. 20). In particular, the binding site for ZFN 13 was truncated by 2 bp and the binding for ZFN 12 was deleted along with additional downstream sequence. Surprisingly, the binding site for the 17/18 pair was intact, indicating that the deletion was a result of cleavage by the 12/13 ZFN pair (see FIG. 21).

### Example 19

### Genome Editing of Cauxin in Cat Cells

Pairs of ZFNs that target regions of the cauxin locus were designed and tested in cat cells as detailed above. TABLE 9 presents the amino acid sequence of the zinc finger helices and DNA binding site of each active ZFN.

TABLE 9
Cauxin ZFNs

| | | | DNA binding site | |
|---|---|---|---|---|
| | Sequence of Zinc | SEQ ID | (Contact sites in | SEQ ID |
| Name | Finger Helices | NO: | uppercase) | NO: |
| 1 | QSGNLAR LAYDRRK | 69 | atCCGGCTGGACCG | 77 |
| (exon 1) | RSDTLSE QSSHLAR | | TCTGAActcctagc | |
| | QSSDLSR RRDTLLD | | | |
| 2 | QSGDLTR NKHHRNR | 70 | agTGGGATGTGGGT | 78 |
| (exon 1) | RSDALAR TSGNLTR | | GCAcccaggccgga | |
| | RRYYLRL | | | |
| 9 | RSDNLAR WRGDRVK | 71 | caGCAGCTGGCCCT | 79 |
| (exon 2) | DRSHLAR QSSDLSR | | GAGgggacacacag | |
| | QSGDLTR | | | |
| 10 | RSDNLSE SSRNLAS | 72 | tgCACCAGtGAGGAG | 80 |
| (exon 2) | RSANLAR RSDNLTR | | CACCAGgctggga | |
| | RSDNLSE SSRNLAS | | | |
| 17 | DSSDRKK QSSDLSR | 73 | taCAGTGGTTTGCTT | 81 |
| (exon 2) | YHWYLKK RSDHLSQ | | CCcccggacccat | |
| | TSAN RTT | | | |
| 18 | QSGNLAR WLSSLGI | 74 | ggGAAGCAcCATGC | 82 |
| (exon 2) | DRSDLSR LRFNLRN | | CTGTGAAcatgttc | |
| | QSGDLTR QSGNLAR | | | |
| 29 | DRSNLSR DAFTRTR | 75 | agGCAGCCAAGGCG | 83 |
| (exon 4) | RSDNLSV ERGTLAR | | GACccatcgaagga | |
| | QSGDLTR | | | |
| 30 | TNHGLNE TSSNLSR | 76 | gaGGACGTGCTGAT | 84 |
| (exon 4) | QSSDLSR HKYHLRS | | CGTgactacccagt | |
| | QSGHLSR | | | |

FIG. 22 presents results from a Cel-1 assay in which cleavage of the cauxin locus by the 1/2 pair, the 9/10 pair, and the 17/18 ZFN pairs were confirmed. FIG. 23 Illustrates cleavage of the cauxin locus by the 29/30 ZFN pair.

### Example 20

### Genome Editing of Agouti in Model Organism Cells

Zinc finger nuclease (ZFN)-mediated genome editing may be tested in the cells of a model organism such as an ovine using a ZFN that binds to the chromosomal sequence of a hair color-related gene of the ovine cell such as MSH receptor proteins, tyrosinase (TYR), tyrosinase-related protein 1 (TYRP1), agouti signaling protein (ASIP), melanophilin (MLPH). The particular coat color-related gene to be edited may be a gene having identical DNA binding sites to the DNA binding sites of the corresponding ovine homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into ovine cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected hair color-related gene locus in ovine cells using a ZFN.

### Example 21

### Genome Editing of Agouti in Model Organism Embryos

The embryos of a model organism such as an ovine may be harvested using standard procedures and injected with capped, polyadenylated mRNA encoding a ZFN similar to that described in Example 20. The ovine embryos may be at the 2-4 cell stage when microinjected. Control embryos were injected with 0.1 mM EDTA. The frequency of ZFN-induced double strand chromosomal breaks was estimated using the Cel-1 assay as described in Example 20. The cutting efficiency may be estimated using the Cel-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

### Example 22

### Genome Editing of FibH in Model Organism Cells

Zinc finger nuclease (ZFN)-mediated genome editing may be tested in the cells of a model organism such as silkworm, Bombyx mori, using a ZFN that binds to the chromosomal sequence of a silkworm fiber related gene of the silkworm cell such as Fibroin heavy chain (FibH), Fibroin light chain (FibL), fibrohexamerin P25, Sericin (Seri), Cry toxin receptor (BtR175), Cytochrome P450 (CYP4, CYP6, CYP9). The particular silk fiber-related gene to be edited may be a gene having identical DNA binding sites to the DNA binding sites of the corresponding insect, such as spider homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into silkworm, Bombyx mori, cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected cognition-related gene locus in silkworm, Bombyx mori, cells using a ZFN.

### Example 23

### Genome Editing of FibH in Model Organism Embryos

The embryos of a model organism such as silkworm, Bombyx mori, egg embryo may be harvested using standard procedures and injected with capped, polyadenylated mRNA encoding a ZFN similar to that described in Example 22. The silkworm, Bombyx mori, egg embryos may be at the 2-4 cell stage when microinjected. Control embryos were injected with 0.1 mM EDTA. The frequency of ZFN-induced double strand chromosomal breaks was estimated using the Cel-1 assay as described in Example 22. The cutting efficiency may be estimated using the CEl-1 assay results.

The development of the embryos following microinjection may be assessed. Embryos injected with a small volume ZFN mRNA may be compared to embryos injected with EDTA to determine the effect of the ZFN mRNA on embryo survival to later stage.

### Example 24

### TUBA1B Promoter

The following example details use of a tubulin promoter to regulate the expression of heterologous protein(s). TUBA1B, which codes for tubulin alpha-1B, was chosen as the target chromosomal sequence. A pair of zinc finger nucleases (ZFNs) was designed to target a location in the TUBA1B coding region. For more additional information see Science (2009) 325:433, which is incorporated by reference herein in its entirety. One ZFN was designed to bind the sequence 5′ CTTCGCCTCCTAATC 3′ (SEQ ID NO:86), and the other ZFN was designed to bind the sequence 5′ CACTATGGTGAGTAA 3′ (SEQ ID NO:87) (FIG. 24A). Upon binding, the ZFN pair introduces a double-stranded break in the sequence 5′ CCTAGC 3′ that lies between the two ZFN recognition sequences. Capped, polyadenylated mRNAs encoding the ZFN pair were produced using known molecular biology techniques.

The gene of interest (i.e., SH2 biosensor) comprised a sequence encoding GFP linked to two SH2 domains and a 2A peptide domain (see FIG. 24B). A plasmid (FIG. 25) was constructed to serve as donor polynucleotide for the targeted integration of the SH2 biosensor sequence into the TUBA1B locus of a human cell line. The plasmid comprised the SH2 biosensor coding sequence flanked by 1 Kb and 700 bp of TUBA1B locus sequence upstream and downstream of the cut site introduced by the ZFN pair. The plasmid was designed such that the SH2 biosensor coding sequence would be integrated in-frame with the endogenous sequence just downstream of the tubulin start codon. Upon activation of the TUBA1B locus, two separate proteins are made, as depicted in FIG. 24B.

The donor plasmid and the pair of RNAs encoding ZFNs were transfected into U2OS, A549, K562, HEK293, or HEK293T cells. The nucleic acid mixture comprised one part donor DNA to one part ZFN RNAs. The transfected cells were then cultured under standard conditions. Analysis of individual cell clones revealed GFP fluorescence, indicating the expression of the heterologous biosensor. Western analysis confirmed that expression of α-tubulin was not affected by the targeted integration (FIG. 24C).

Another donor plasmid was constructed to allow insertion if a Grb2-containing biosensor (i.e., GFP-2xSH2-Grb2-2A). The Grb2-containing biosensor is activated by EGF and undergoes nuclear translocation. A549 cells were transfected with the nucleic acids and cultured to allow integration and expression of the TUBA1B locus. Cells were exposed to 100 ng/ml of EGF and imaged. FIG. 26 presents a time course of the translocation of the SH2 biosensor.

### Example 25

### ACTB Promoter

The following example was designed to test the use of a stronger promoter. A well known strong promoter is within the ACTB locus, which encodes β-actin. A pair of ZFNs was designed to target the ACTB locus. One ZFN was designed to bind the sequence 5′ GTCGTCGACAACGGCTCC 3′ (SEQ ID NO:88), and the other ZFN was designed to bind the sequence 5′ TGCAAGGCCGGCTTCGCGG 3′ (SEQ ID NO:89). Upon binding, the ZFN pair introduces a double-stranded break in the sequence 5′ GGCATG 3′ that lies between the two ZFN recognition sequences.

A donor plasmid was designed to provide the SH2 biosensor sequence, as well as tag the endogenously produced β-actin (i.e., GFP-2x-SH2-2A-RFP) (FIG. 27). The nucleic acids were introduced into cells, and two fluorescent proteins were made (i.e., GFP biosensor and RFP-tagged actin). The fluorescence of each protein was monitored using fluorescent microscopy.

To better monitor the different fluorescent proteins, another donor plasmid was constructed to contain a Grb2-containing biosensor. Accordingly, the donor plasmid comprised: GFP-2xSH2-Grb2-2A-RFP. A549 cells were transfected with the nucleic acids and cultured to allow integration and expression of the ACTB locus. Cells were exposed to 100 ng/ml of EGF and imaged. FIG. 28 presents a time course of the translocation of the GFP-Grb2 biosensor and the location of RFP-actin. The amount biosensor produced was so high that there were high levels of unbound or “free” biosensor, thereby drastically increasing the amount of background fluorescence.

### Example 26

### LMNB1 Promoter

To target the LMNB1 locus, which codes for lamin B1 protein, another pair of ZFNs was made. One ZFN was designed to bind the sequence 5′ CCTCGCCGCCCCGCT 3′ (SEQ ID NO:90), and the other ZFN was designed to bind the sequence 5′ GCCGCCCGCCATGGCG 3′ (SEQ ID NO:91). Upon binding, the ZFN pair introduces a double-stranded break in the sequence 5′ GTCTCC 3′ that lies between the two recognition sequences.

A donor plasmid may be constructed to comprise a sequence encoding a biosensor protein that is flanked by LMNB1 sequences upstream and downstream of the ZFN cleavage site. The nucleic acids encoding the ZFNs and the donor plasmid may be introduced into cells, and the cells may be monitored as detailed above.

### Example 27

### Identification of ZFNs that Edit the LRRK2 Locus

The LRRK2 gene in rat was chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The LRRK2 gene region (XM_{—}235581) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-tgGGTCATGAAGTGGGGGTGagtgctgt-3′ (SEQ ID NO:94; contact sites in uppercase) and 5′-gaGCCCTGTACCTGGCTGTCtacgacct′3′ (SEQ ID NO:95) cleaved within the LRRK2 locus.

### Example 28

### Editing the LRRK2 Locus in Rat Embryos

Capped, polyadenylated mRNA encoding the active pair of ZFNs was microinjected into fertilized rat embryos using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the LRRK2 locus was PCR amplified using appropriate primers. The amplified DNA was subcloned into a suitable vector and sequenced using standard methods. FIG. 29 illustrates edited LRRK2 loci in two founder animals. One animal had a 10 bp deletion in the target sequence of exon 30, and the second animal had an 8 bp deletion in the target sequence of exon 30. These deletions disrupt the reading frame of the LRRK2 coding region.

### Example 29

### Identification of ZFNs that Edit the SNCA Locus

ZFNs that may edit the SNCA (α-synuclein) locus were designed by scanning the rat SNCA locus (NM_{—}019169) for putative zinc finger binding sites. The ZFNs were assembled and tested essentially as described in Example 27. This analysis revealed that the ZFN pair targeted to bind 5′-agTCAGCACAGGCATGTccatgttgagt-3′ (SEQ ID NO:96) and 5′-ccTCTGGGGTAGTGAACAGGtctcccac-3′ (SEQ ID NO:97) cleaved within SNCA gene.

### Example 30

### Identification of ZFNs that Edit the DJ-1 Locus

ZFNs with activity at the DJ-1 locus were identified as described above. That is, the rat DJ-1 gene (NM_{—}019169) was scanned for putative zinc finger binding sites, and ZFNs were assembled and tested essentially as described in Example 27. It was found that the ZFN pair targeted to bind 5′-aaGCCGACTAGAGAGAGaacccaaacgc-3′ (SEQ ID NO:98) and 5′-gtGAAGGAGATcCTCAAGgagcaggaga-3′ (SEQ ID NO:99) edited the DJ-1 locus.

### Example 31

### Identification of ZFNs that Edit the Parkin Locus

To identify ZFNs that target and cleave the Parkin locus, the rat Parkin gene (NM_{—}020093) was scanned for putative zinc finger binding sites. The ZFNs pairs were assembled and tested essentially as described in Example 27. This analysis revealed that the ZFN pair targeted to bind 5′-gaACTCGGaGTTTCCCAGgctggacctt-3′ (SEQ ID NO:100) and 5′-gtGCGGCACCTGCAGACaagcaaccctc-3′ (SEQ ID NO:101) cleaved within the Parkin gene.

### Example 32

### Identification of ZFNs that Edit the PINK1 Locus

ZFNs with activity at the PINK1 locus were identified essentially as described above. The rat PINK1 gene (NM_{—}020093) was scanned for putative zinc finger binding sites. The ZFNs were assembled and tested essentially as described in Example 27. This analysis revealed that the ZFN pair targeted to bind 5′-ggGTAGTAGTGTGGGGGtagcatgtcag-3′ (SEQ ID NO:102) and 5′-aaGGCCTGgGCCACGGCCGCAcactctt-3′ (SEQ ID NO:103) edited the PINK1 gene.

The table below presents the amino acid sequences of helices of the active ZFNs.

| | | SEQ ID |
|---|---|---|
| Name | Sequence of Zinc Finger Helices | NO: |
| SNCA | WRSCRSA QSGSLTR RSDNLRE QSGSLTR | 104 |
| | QSADRTK | |
| SNCA | RSDHLSA DRSNRKT RSAALSR QSGSLTR | 105 |
| | RSDHLSE RKHDRTK | |
| DJ-1 | RSDALSV QSQHRTT RSDNLSV DRSNLTR | 106 |
| | DRSDLSR | |
| DJ-1 | RSDNLST DNSSRIT TSSNLSR QSGHLQR | 107 |
| | QSGNLAR | |
| LRRK2 | ASTGLIR RSDHLSR RSDALSR QSGNLAR | 108 |
| | NNTQLIE TSSILSR | |
| LRRK2 | DRSALSR QSSDLRR RSDVLSA DRSNRIK | 109 |
| | RSDSLSA DRSSRTK | |
| Parkin | RSDNLSQ ASNDRKK HRSSLRR RSDHLSE | 110 |
| | ARSTRTN | |
| Parkin | DRSNLSR QSGDLTR HKTSLKD QSGDLTR | 111 |
| | RSDDLTR | |
| PINK1 | RSSHLSR RSDHLST ASSARKT QSGALAR | 112 |
| | QSGSLTR | |
| PINK1 | QSGDLTR DRSDLSR RSDTLSV DNSTRIK | 113 |
| | RSDALSV DSSHRTR | |

### Example 33

### Genome Editing of ApoE Locus

Zinc finger nucleases (ZFNs) that target and cleave the ApoE locus of rabbit may be designed, assembled, and validated using strategies and procedures previously described (see Geurts et al. Science (2009) 325:433). ZFN design made use of an archive of pre-validated 1-finger and 2-finger modules. The rabbit ApoE gene region may be scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rabbit cells. Control cells may be injected with mRNA encoding GFP. Active ZFN pairs may be identified by detecting ZFN-induced double strand chromosomal breaks using the Cel-1 nuclease assay. This assay may detect alleles of the target locus that deviate from wild type as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch may be cleaved by the surveyor nuclease Cel-1, and the cleavage products can be resolved by gel electrophoresis. This assay may identify a pair of active ZFNs that edited the ApoE locus.

To mediate editing of the ApoE gene locus in animals, fertilized rabbit one cell embryos may be microinjected with mRNA encoding the active pair of ZFNs using standard procedures (e.g., see Geurts et al. (2009) supra). The injected embryos may be either incubated in vitro, or transferred to pseudopregnant female rabbits to be carried to parturition. The resulting embryos/fetus, or the toe/tail clip of live born animals may be harvested for DNA extraction and analysis. DNA may be isolated using standard procedures. The targeted region of the ApoE locus may be PCR amplified using appropriate primers. The amplified DNA may be subcloned into a suitable vector and sequenced using standard methods.

### Example 34

### Genome Editing of FAH in a Model Organism

ZFN-mediated genome editing may be used to study the effects of a “knock-out” mutation in a rabbit or human disease-related chromosomal sequence, such as a chromosomal sequence encoding the fumarylacetoacetate hydrolase (FAH), in a genetically modified model animal and cells derived from the animal. Such a model animal may be a rabbit. In general, ZFNs that bind to the rabbit chromosomal sequence encoding the fumarylacetoacetate hydrolase associated with rabbit immunodeficiency may be used to introduce a deletion or insertion such that the coding region of the FAH gene is disrupted such that a functional FAH protein may not be produced.

Suitable fertilized embryos may be microinjected with capped, polyadenylated mRNA encoding the ZFN essentially as detailed above in Example 33. The frequency of ZFN-induced double strand chromosomal breaks may be determined using the Cel-1 nuclease assay, as detailed above. The sequence of the edited chromosomal sequence may be analyzed as described above. The development of immunodeficiency symptoms and disorders caused by the fumarylacetoacetate hydrolase “knock-out” may be assessed in the genetically modified rabbit or progeny thereof. Furthermore, molecular analyses of immunodeficiency-related pathways may be performed in cells derived from the genetically modified animal comprising a FAH “knock-out”.

### Example 35

### Generation of a Humanized Rabbit Expressing a Mutant Form of Human cTnl

Familial hypertrophic cardiomyopathy (FHC) displays an autosomal dominant mode of inheritance and a diverse genetic etiology. FHC or a phenocopy can be caused by multiple mutations in genes encoding various contractile, structural, channel and kinase proteins. Commonly, arrhythmias, particularly ventricular tachycardia and fibrillation associated with FHC would lead to sudden death. A single base change at cTnl locus leads to alteration of a disease-associated protein, cardiac troponin. ZFN-mediated genome editing may be used to generate a humanized rabbit wherein the rabbit cTnl locus is replaced with a mutant form of the human cTnl locus comprising one or more mutations. Such a humanized rabbit may be used to study the development of the diseases associated with the human FHC. In addition, the humanized rabbit may be used to assess the efficacy of potential therapeutic agents targeted at the pathway leading to FHC comprising cTnl.

The genetically modified rabbit may be generated using the methods described in the Examples above. However, to generate the humanized rabbit, the ZFN mRNA may be co-injected with the human chromosomal sequence encoding the mutant cardiac troponin protein into the rabbit embryo. The rabbit chromosomal sequence may then be replaced by the mutant human sequence by homologous recombination, and a humanized rabbit expressing a mutant form of the cardiac troponin protein may be produced.

### Example 36

### Genome Editing of PRPN in Model Organism Cells

Zinc finger nuclease (ZFN)-mediated genome editing may be tested in the cells of a model organism such as a bovine using a ZFN that binds to the chromosomal sequence of a prion protein gene of the bovine cell such PRPN. The particular gene to be edited may be a gene having identical DNA binding sites to the DNA binding sites of the corresponding bovine homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into bovine cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected PRPN gene locus in bovine cells using a ZFN.

### Example 37

### Genome Editing of PRPN in Model Organism Embryos

The embryos of a model organism such as a bovine may be harvested using standard procedures and injected with capped, polyadenylated mRNA encoding a ZFN similar to that described in Example 36. The bovine embryos may be at the one cell stage when microinjected. Control embryos may be injected with 0.1 mM EDTA. The frequency of ZFN-induced double strand chromosomal breaks may be estimated using the Cel-1 assay as described in Example 36. The cutting efficiency may be estimated using the CEl-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

### Example 38

### Identification of ZFNs that Edit the ApoE Locus

The ApoE gene was chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The rat ApoE gene region (NM_{—}138828) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′ aaGCGGTTCAGGGCCTGctcccagggtt-3′ (SEQ ID NO: 117; contact sites in uppercase) and 5′ ggGATTACCTGcGCTGGGtgcagacgct-3′ (SEQ ID NO: 118) cleaved within the ApoE locus

### Example 39

### Editing the ApoE Locus in Rat Embryos

Capped, polyadenylated mRNA encoding the active pair of ZFNs was microinjected into fertilized rat embryos using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the ApoE locus was PCR amplified using appropriate primers. The amplified DNA was subcloned into a suitable vector and sequenced using standard methods. FIG. 30 presents two edited ApoE loci. One animal had a 16 bp deletion in the target sequence of exon 2, and a second animal had a 1 bp deletion in the target sequence of exon 2. These deletions disrupt the reading frame of the ApoE coding region.

### Example 40

### Identification of ZFNs that Edit the Leptin Locus

ZFNs that target and cleave the leptin gene in rat were identified essentially as described above. The rat leptin gene (NM_{—}013076) was scanned for putative zinc finger binding sites. ZFNs were assembled and tested essentially as described in Example 38. This assay revealed that the ZFN pair targeted to bind 5′-gtGGATAGGCACAGcttgaacataggac-3′ (SEQ ID NO: 119; contact sites in uppercase) and 5′ aaGTCCAGGATGACACCaaaaccctcat-3′ (SEQ ID NO: 120) cleaved within the leptin locus

### Example 41

### Editing the Leptin Locus in Rat Embryos

Rat embryos were microinjected with mRNA encoding the active pair of leptin ZFNs essentially as described in Example 39. The injected embryos were incubated and DNA was extracted from the resultant animals. The targeted region of the leptin locus was PCR amplified using appropriate primers. The amplified DNA was subcloned into a suitable vector and sequenced using standard methods. FIG. 31 presents an edited leptin locus, in which a 151 bp region was deleted from the 3′ end of exon 1 and the 5′ end of intron 1.

### Example 42

### Editing the Pten Locus in Rat Embryos

ZFNs that target and cleave the Pten locus in rats were designed and tested for activity essentially as described above in Example 38. An active pair of ZFNs was identified. The DNA binding sites were 5′-CCCCAGTTTGTGGTCtgcca-3′ SEQ ID NO:121) and 5′-gcTAAAGGTGAAGATCTA-3′ (SEQ ID NO:122). Capped, polyadenylated mRNA encoding the active pair may be microinjected into rat embryos and the resultant embryos may b analyzed as described in Example 39. Accordingly, the Pten locus may be edited to contain a deletion or an insertion such that the coding region is disrupted and no functional gene product is made.

### Example 43

### Genome Editing of Canca1C in Model Organism Cells

Zinc finger nuclease (ZFN)-mediated genome editing may be tested in the cells of a model organism such as a rat using ZFN that binds to the chromosomal sequences of a cardiovascular-related gene of the rat cell such as Canca1C, Sod1, Pten, Ppar(alpha), and combinations thereof. The particular chromosomal sequence involved in cardiovascular disease to be edited may be a gene having identical DNA binding sites to the DNA binding sites of the corresponding human homologue of the gene. Capped, polyadenylated mRNA encoding the ZFN may be produced using known molecular biology techniques, including, but not limited to, a technique substantially similar to the technique described in Science (2009) 325:433, which is incorporated by reference herein in its entirety. The mRNA may be transfected into rat cells as well as human K562 cells, assuming the K562 cells have identical DNA binding sites. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected cognition-related gene locus in human and rat cells using a ZFN.

### Example 44

### Genome Editing of Canca1C in Model Organism Embryos

The embryos of a model organism such as a rat may be harvested using standard procedures and injected with capped, polyadenylated mRNA encoding a ZFN similar to that described in Example 43. The rat embryos may be at the single cell stage when microinjected. Control embryos may be injected with 0.1 mM EDTA. The frequency of ZFN-induced double strand chromosomal breaks may be estimated using the Cel-1 assay as described in Example 43. The cutting efficiency may be estimated using the CEl-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

The table below presents the amino acid sequences of helices of the active ZFNs.

| | | SEQ ID |
|---|---|---|
| Name | Sequence of Zinc Finger Helices | NO: |
| ApoE | RSDALSV DSSHRTR RSDNLSE TSGSLTR RSDDLTR | 123 |
| ApoE | RSDHLSR QSSDLRR RSDVLSA DRSNRIK TSSNLSR | 124 |
| Leptin | RSDALSE QNATRTK RSDYLST QNAHRKT | 125 |
| Leptin | DQSTLRN DRSNLSR TSANLSR RSDNLSE DRSALAR | 126 |

### Example 45

### Genome Editing of Myostatin/GDF8, CD163 or Sialoadhesin in Model Organism Cells

Zinc finger nuclease (ZFN)-mediated genome editing may be tested in the cells of a model organism such as an porcine using a ZFN that binds to the chromosomal sequence of a hair color-related gene of the porcine cell such as MC1R, MSH receptor proteins, tyrosinase (TYR), tyrosinase-related protein 1 (TYRP1), agouti signaling protein (ASIP), melanophilin (MLPH). The particular coat color-related gene to be edited may be a gene having identical DNA binding sites to the DNA binding sites of the corresponding porcine homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into porcine cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected myostatin/GDF8, CD163 or sialoadhesin gene locus in porcine cells using a ZFN.

### Example 46

### Genome Editing of HAL, RN, ESR, IGF2, GHRH, H-FABP, GH, IGF1, PIT1, GHRHR or GHR in Model Organism Embryos

The embryos of a model organism such as a porcine may be harvested using standard procedures and injected with capped, polyadenylated mRNA encoding a ZFN similar to that described in Example 45. The porcine embryos may be at the 2-4 cell stage when microinjected. Control embryos were injected with 0.1 mM EDTA. The frequency of ZFN-induced double strand chromosomal breaks was estimated using the Cel-1 assay as described in Example 45. The cutting efficiency may be estimated using the CEl-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

### Example 47

### Genome Editing of Can f 1 Locus

Zinc finger nucleases (ZFNs) that target and cleave the Can f 1 locus of canine may be designed, assembled, and validated using strategies and procedures previously described (see Geurts et al. Science (2009) 325:433). ZFN design made use of an archive of pre-validated 1-finger and 2-finger modules. The canine Can f 1 gene region may be scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into canine cells. Control cells may be injected with mRNA encoding GFP. Active ZFN pairs may be identified by detecting ZFN-induced double strand chromosomal breaks using the Cel-1 nuclease assay. This assay may detect alleles of the target locus that deviate from wild type as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch may be cleaved by the surveyor nuclease Cel-1, and the cleavage products can be resolved by gel electrophoresis. This assay may identify a pair of active ZFNs that edited the Can f 1 locus.

To mediate editing of the Can f 1 gene locus in animals, fertilized canine embryos may be microinjected with mRNA encoding the active pair of ZFNs using standard procedures (e.g., see Geurts et al. (2009) supra). The injected embryos may be either incubated in vitro, or transferred to pseudopregnant female canines to be carried to parturition. The resulting embryos/fetus, or the toe/tail clip of live born animals may be harvested for DNA extraction and analysis. DNA may be isolated using standard procedures. The targeted region of the Can f 1 locus may be PCR amplified using appropriate primers. The amplified DNA may be subcloned into a suitable vector and sequenced using standard methods.

### Example 48

### Genome Editing of HCRTR2 in a Model Organism

ZFN-mediated genome editing may be used to study the effects of a “knock-out” mutation in a canine or human disease-related chromosomal sequence, such as a chromosomal sequence encoding the hypocretin receptor protein, in a genetically modified model animal and cells derived from the animal. Such a model animal may be a canine. In general, ZFNs that bind to the canine chromosomal sequence encoding the hypocretin receptor associated with canine narcolepsy may be used to introduce a deletion or insertion such that the coding region of the HCRTR2 gene is disrupted such that a functional hypocretin receptor protein may not be produced.

Suitable fertilized embryos may be microinjected with capped, polyadenylated mRNA encoding the ZFN essentially as detailed above in Example 47. The frequency of ZFN-induced double strand chromosomal breaks may be determined using the Cel-1 nuclease assay, as detailed above. The sequence of the edited chromosomal sequence may be analyzed as described above. The development of narcolepsy symptoms and disorders caused by the hypocretin receptor “knock-out” may be assessed in the genetically modified canine or progeny thereof. Furthermore, molecular analyses of narcolepsy-related pathways may be performed in cells derived from the genetically modified animal comprising a HCRTR2 “knock-out”.

### Example 49

### Generation of a Humanized Canine Expressing a Mutant Form of Human BHD

BHD is a multisystem disorder in humans that has strong similarity to RCND, a naturally occurring inherited canine cancer syndrome. RCND locus overlaps with human BHD locus in genome comparison. A single base change at RCND locus leads to alteration of a disease-associated protein folliculin. ZFN-mediated genome editing may be used to generate a humanized canine wherein the canine RCND locus is replaced with a mutant form of the human BHD locus comprising one or more mutations. Such a humanized canine may be used to study the development of the diseases associated with the mutant human BHD protein. In addition, the humanized canine may be used to assess the efficacy of potential therapeutic agents targeted at the pathway leading to kidney cancer comprising BHD.

The genetically modified canine may be generated using the methods described in the Examples above. However, to generate the humanized canine, the ZFN mRNA may be co-injected with the human chromosomal sequence encoding the mutant BHD protein into the canine embryo. The canine chromosomal sequence may then be replaced by the mutant human sequence by homologous recombination, and a humanized canine expressing a mutant form of the BHD protein may be produced.

### Example 50

### Genome Editing of an Addiction-Related Protein in Model Organism Cells

Zinc finger nuclease (ZFN)-mediated genome editing may be tested in the cells of a model organism such as a rat using a ZFN that binds to the chromosomal sequence of an addiction-related gene of the rat cell such as ABAT (4-aminobutyrate aminotransferase), DRD2 (Dopamine receptor D2), DRD3 (Dopamine receptor D3), DRD4 (Dopamine receptor D4), GRIA1 (Glutamate receptor, ionotropic, AMPA 1), GRIA2 (Glutamate receptor, ionotropic, AMPA 2), GRIN1 (Glutamate receptor, ionotropic, N-methyl D-aspartate 1), GRIN2A (Glutamate receptor, ionotropic, N-methyl D-aspartate 2A), GRM5 (Metabotropic glutamate receptor 5), HTR1B (5-Hydroxytryptamine (serotonin) receptor 1B), PDYN (Dynorphin), or PRKCE (Protein kinase C, epsilon). The particular addiction-related gene to be edited may be a gene having identical DNA binding sites to the DNA binding sites of the corresponding human homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into rat cells as well as human K562 cells, assuming the K562 cells have identical DNA binding sites. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected addiction-related gene locus in human and rat cells using a ZFN.

### Example 51

### Genome Editing of an Addiction-Related Protein in Model Organism Embryos

The embryos of a model organism such as a rat may be harvested using standard procedures and injected with capped, polyadenylated mRNA encoding a ZFN similar to that described in Example 50. The rat embryos may at the single cell stage when microinjected. Control embryos were injected with 0.1 mM EDTA. The frequency of ZFN-induced double strand chromosomal breaks was estimated using the Cel-1 assay as described in Example 50. The cutting efficiency may be estimated using the CEl-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

### Example 52

### Genome Editing of the APP Locus

Zinc finger nucleases (ZFNs) that target and cleave the APP locus of rats were designed, assembled, and validated using strategies and procedures previously described (see Geurts et al. Science (2009) 325:433). ZFN design made use of an archive of pre-validated 1-finger and 2-finger modules. The rat APP gene region was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay identified a pair of active ZFNs that edited the APP locus. The zinc finger binding sites were 5′-GCCAGCACCCCTGACgcag′3-(SEQ ID NO:129) and 5′-tcGACAAGTACCTGGAG′3′ (SEQ ID NO:130).

To mediate editing of the APP gene locus in animals, fertilized rat embryos were microinjected with mRNA encoding the active pair of ZFNs using standard procedures (e.g., see Geurts et al. (2009) supra). The injected embryos were either incubated in vitro, or transferred to pseudopregnant female rats to be carried to parturition. The resulting embryos/fetus, or the toe/tail clip of live animals were harvested for DNA extraction and analysis. DNA was isolated using standard procedures. The targeted region of the APP locus was PCR amplified using appropriate primers. The amplified DNA was subcloned into a suitable vector and sequenced using standard methods. FIG. 32 presents edited APP loci in two founder animals; one had a 292 bp deletion in exon 9 (FIG. 32A) and the other had a 309 bp deletion in exon 9 (FIG. 32B).

### Example 53

### Genome Editing of Cognition-Related Genes in Model Organism Cells

ZFN-mediated genome editing may be tested in the cells of a model organism such as a rat using a ZFN that binds to the chromosomal sequence of a cognition-related gene such as ANK3 (Ankryn 3), APP (Amyloid precursor protein), B2M (Beta-2 microglobulin), BRD1 (Bromodomain containing 1), FMR1 (Fragile X mental retardation 1), MECP2 (Methyl CpG binding protein 2), NGFR (Nerve growth factor receptor), NLGN3 (Neuroligin 3), or NRXN1 (Neurexin 1). ZFNs may be designed and tested essentially as described in Example 52. ZFNs targeted to a specific cognition-related gene may be used to introduce a deletion or insertion such that the coding region of the gene of interest is inactivated.

### Example 54

### Genome Editing of Cognition-Related Genes in Model Organisms

The embryos of a model organism such as a rat may be harvested using standard procedures and injected with capped, polyadenylated mRNA encoding ZFNs that target cognition-related genes, as detailed above in Example 52. Donor or exchange polynucleotides comprising sequences for integration or exchange may be co-injected with the ZFNs. The edited chromosomal regions in the resultant animals may be analyzed as described above. The modified animals may be phenotypically analyzed for changes in behavior, learning, etc. Moreover, the genetically modified animal may be used to assess the efficacy of potential therapeutic agents for the treatment of cognition-related disorders.

### Example 55

### Genome Editing of CCR2 in a Model Organism

Zinc finger nuclease (ZFN)-mediated genome editing may be used to study the effects of a “knock-out” mutation in an inflammation-related chromosomal sequence, such as a chromosomal sequence encoding the CCR2 protein, in a genetically modified model animal and cells derived from the animal. Such a model animal may be a rat. In general, ZFNs that bind to the rat chromosomal sequence encoding the inflammation-related protein CCR2 may be used to introduce a non-sense mutation into the coding region of the CCR2 gene, such that an active CCR2 protein may not be produced.

Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into rat embryos. The rat embryos may be at the single cell stage when microinjected. Control embryos may be injected with 0.1 mM EDTA. The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The development of the embryos following microinjection, and the development of inflammation-related symptoms and disorders caused by the CCR2 “knock-out” may be assessed in the genetically modified rat. For CCR2, inflammation-related symptoms and disorders may include development of rheumatoid arthritis and an altered inflammatory response against tumors. The results may be compared to the control rat injected with 0.1 mM EDTA, where the chromosomal region encoding the CCR2 protein is not altered. In addition, molecular analysis of inflammation-related pathways may be performed in cells derived from the genetically modified animal comprising a CCR2 “knock-out”.

### Example 56

### Generation of a Humanized Rat Expressing a Mutant Form of Human Perforin-1

Missense mutations in perforin-1, a critical effector of lymphocyte cytotoxicity, lead to a spectrum of diseases, from familial hemophagocytic lymphohistiocytosis to an increased risk of tumorigenesis. One such mutation is the V50M missense mutation where the valine amino acid at position 50 in perforin-1 is replaced with methionine. ZFN-mediated genome editing may be used to generate a humanized rat wherein the rat PRF1 gene is replaced with a mutant form of the human PRF1 gene comprising the V50M mutation. Such a humanized rat may be used to study the development of the diseases associated with the mutant human perforin-1 protein. In addition, the humanized rat may be used to assess the efficacy of potential therapeutic agents targeted at the inflammatory pathway comprising perforin-1.

The genetically modified rat may be generated using the methods described in Example 55 above. However, to generate the humanized rat, the ZFN mRNA may be co-injected with the human chromosomal sequence encoding the mutant perforin-1 protein into the rat embryo. The rat chromosomal sequence may then be replaced by the mutant human sequence by homologous recombination, and a humanized rat expressing a mutant form of the perforin-1 protein may be produced.

### Example 57

### Editing the Pten Locus

ZFNs that target and cleave the Pten locus in rats were designed and tested for activity essentially as described above in Example 55. An active pair of ZFNs was identified. The DNA binding sites were 5′-CCCCAGTTTGTGGTCtgcca-3′ (SEQ ID NO:135) and 5′-gcTAAAGGTGAAGATCTA-3′ (SEQ ID NO:136). Capped, polyadenylated mRNA encoding the active pair may be microinjected into rat embryos and the resultant embryos may be analyzed as described in Example 55. Accordingly, the Pten locus may be edited to contain a deletion or an insertion such that the coding region is disrupted and no functional gene product is made.

### Example 58

### Identification of ZFNs that Edit the Rag1 Locus

The Rag1 gene was chosen for zinc finger nuclease (ZFN) mediated genome editing. ZFNs were designed, assembled, and validated using strategies and procedures described in the examples above. ZFN design made use of an archive of pre-validated 1-finger and 2-finger modules. The rat Rag1 gene region (XM_{—}001079242) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites. Capped, polyadenylated mRNA encoding each pair of ZFNs was produced and transfected into rat cells. Control cells were injected with mRNA encoding GFP. Active ZFN pairs were identified by detecting ZFN-induced double strand chromosomal breaks using the Cel-1 nuclease assay. This assay revealed that the ZFN pair targeted to bind 5′-ttCCTTGGGCAGTAGACctgactgtgag-3′ (SEQ ID NO:137; contact sites in upper case) and 5′-gtGACCGTGGAGTGGCAcccccacacac-3′ (SEQ ID NO: 138) cleaved within the Rag1 gene.

### Example 59

### Editing the Rag1 Locus

Capped, polyadenylated mRNA encoding the active pair of ZFNs was microinjected into fertilized rat embryos as described in the examples above. The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the Rag1 locus was PCR amplified using appropriate primers. The amplified DNA was subcloned into a suitable vector and sequenced using standard methods. FIG. 33 presents DNA sequences of edited Rag1 loci in two animals (SEQ ID NOS: 131 and 132). One animal had a 808 bp deletion in exon 2, and a second animal had a 29 bp deletion in the target sequence of exon 2. These deletions disrupt the reading frame of the Rag1 coding region.

### Example 60

### Identification of ZFNs that Edit the Rag2 Locus

ZFNs that target and cleave the Rag2 gene were identified essentially as described above. The rat Rag2 gene (XM_{—}001079235) was scanned for putative zinc finger binding sites. ZFNs were assembled and tested essentially as described in Example 55. This assay revealed that the ZFN pair targeted to bind 5′-acGTGGTATATaGCCGAGgaaaaagtgt-3′ (SEQ ID NO: 139; contact sites in uppercase) and 5′-atACCACGTCAATGGAAtggccatatct-′3′ (SEQ ID NO: 140) cleaved within the Rag2 locus.

### Example 61

### Editing the Rag2 Locus

Rat embryos were microinjected with mRNA encoding the active pair of Rag2 ZFNs essentially as described in Example 56. The injected embryos were incubated and DNA was extracted from the resultant animals. The targeted region of the Rag2 locus was PCR amplified using appropriate primers. The amplified DNA was subcloned into a suitable vector and sequenced using standard methods. FIG. 34 presents DNA sequences of edited Rag2 loci in two animals. One animal had a 13 bp deletion in the target sequence in exon 3, and a second animal had a 2 bp deletion in the target sequence of exon 3. These deletions disrupt the reading frame of the Rag2 coding region.

### Example 62

### Identification of ZFNs that Edit the FoxN1 Locus

ZFNs that target and cleave the FoxN1 gene were identified essentially as described above in Example 55. The rat FoxN1 gene (XM_{—}220632) was scanned for putative zinc finger binding sites. ZFNs were assembled and tested essentially as described in Example 55. This assay revealed two pairs of active ZFNs that cleaved within the FoxN1 locus: a first pair targeted to bind 5′-ttAAGGGCCATGAAGATgaggatgctac-3′ (SEQ ID NO: 141; contact sites in uppercase) and 5′-caGCAAGACCGGAAGCCttccagtcagt-′3′ (SEQ ID NO: 142); and a second pair targeted to bind 5′-ttGTCGATTTTGGAAGGattgagggccc-3′ (SEQ ID NO: 143) and 5′-atGCAGGAAGAGCTGCAgaagtggaaga-′3′ (SEQ ID NO: 144)

### Example 63

### Identification of ZFNs that Edit the DNAPK Locus

ZFNs that target and cleave the DNAPK gene were identified essentially as described above in Example 55. The rat DNAPK gene (NM_{—}001108327) was scanned for putative zinc finger binding sites. ZFNs were assembled and tested essentially as described in Example 55. This assay revealed that the ZFN pair targeted to bind 5′-taCACAAGTCCtTCTCCAggagctagaa-3′ (SEQ ID NO: 145; contact sites in uppercase) and 5′-acAAAGCTTATGAAGGTcttagtgaaaa-′3′ (SEQ ID NO: 146) cleaved within the DNAPK locus.

The table below presents the amino acid sequences of helices of the active ZFNs.

| | | SEQ |
|---|---|---|
| | | ID |
| Name | Sequence of Zinc Finger Helices | NO: |
| RAG1 | DRSNLSR QSGSLTR ERGTLAR RSDHLTT HKTSLKD | 147 |
| RAG1 | QNATRIK RSDALSR QSGHLSR RSADLTE DRANLSR | 148 |
| RAG2 | RSDNLSR DSSTRKK NSGNLDK QSGALAR | 149 |
| | RSDALAR | |
| RAG2 | QSGNLAR RSDSLSV QSADRTK RSDTLST DRKTRIN | 150 |
| FOXN1 | TSGNLTR QSGNLAR LKQNLDA DRSHLTR | 151 |
| | RLDNRTA | |
| FOXN1 | DRSDLSR QSGNLAR RSDTLSE QRQHRTT | 152 |
| | QNATRIK | |
| FOXN1 | RSDHLSA QSGHLSR DSESLNA TSSNLSR | 153 |
| | DRSSRKR | |
| FOXN1 | QSGSLTR QSSDLRR QRTHLTQ QSGHLQR | 154 |
| | QSGDLTR | |
| DNAPK | QSGDLTR SSSDRKK DSSDRKK RSDNLST | 155 |
| | DNSNRIN | |
| DNAPK | TSGHLSR QSGNLAR HLGNLKT QSSDLSR | 156 |
| | QSGNRTT | |

### Example 64

### Genome Editing of Oct 1 in a Model Organism

ZFN-mediated genome editing may be used to study the effects of a “knock-out” mutation in an AD-related chromosomal sequence, such as a chromosomal sequence encoding the Oct 1 protein, in a genetically modified model animal and cells derived from the animal. Such a model animal may be a rat. In general, ZFNs that bind to the rat chromosomal sequence encoding the Oct 1 protein associated with AD may be used to introduce a deletion or insertion such that the coding region of the Oct 1 gene is disrupted such that a functional Oct 1 protein may not be produced.

Suitable fertilized embryos which may be at the single-cell stage may be microinjected with capped, polyadenylated mRNA encoding the ZFN. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the Oct 1 “knock-out” may be assessed in the genetically modified rat or progeny thereof. Furthermore, molecular analyses of AD-related pathways may be performed in cells derived from the genetically modified animal comprising an ErbB4 “knock-out”.

### Example 65

### Generation of a Humanized Rat Expressing a Mutant Form of Human Genes Involved in ADME and Toxicology

Mutations in any of the chromosomal sequences involved in ADME and toxicology can be used in the generation of a humanized rat expressing a mutant form of the gene. The genes can be Oct 1, Oct 2, Hfe2, Ppar(alpha), and combinations thereof. ZFN-mediated genome editing may be used to generate a humanized rat wherein the rat gene is replaced with a mutant form of the human gene comprising the mutation. Such a humanized rat may be used to study the development of the diseases associated with the mutant human protein encoded by the gene of interest. In addition, the humanized rat may be used to assess the efficacy of potential therapeutic agents targeted at the pathway leading to AD comprising the gene of interest.

The genetically modified rat may be generated using the methods described in the Example above. However, to generate the humanized rat, the ZFN mRNA may be co-injected with the human chromosomal sequence encoding the mutant protein into the rat embryo. The rat chromosomal sequence may then be replaced by the mutant human sequence by homologous recombination, and a humanized rat expressing a mutant form of the protein may be produced.

### Example 66

### Identification of ZFNs that Edit the Mdr1a Locus

The Mdr1a gene was chosen for zinc finger nuclease (ZFN) mediated genome editing. ZFNs were designed, assembled, and validated using strategies and procedures previously described (see Geurts et al., Science (2009) 325:433). ZFN design made use of an archive of pre-validated 1-finger and 2-finger modules. The rat Mdr1a gene region (NM_{—}133401) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and re-annealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-acAGGGCTGATGGCcaaaatcacaagag-3′ (SEQ ID NO: 164; contact sites in uppercase) and 5′-ttGGACTGTCAGCTGGTatttgggcaaa-′3′ (SEQ ID NO: 165) cleaved within the Mdr1a locus.

### Example 67

### Editing the Mdr1a Locus

Capped, polyadenylated mRNA encoding the active pair of ZFNs was microinjected into fertilized rat embryos using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the Mdr1a locus was PCR amplified using appropriate primers. The amplified DNA was subcloned into a suitable vector and sequenced using standard methods. FIG. 35 presents DNA sequences of edited Mdr1a loci in two animals. One animal had a 20 bp deletion in the target sequence in exon 7, and a second animal had a 15 bp deletion and a 3 bp insertion in the target sequence of exon 7. The edited loci harbored frameshift mutations and multiple translational stop codons.

Western analyses were performed to confirm that the Mdr1a locus was inactivated such that no Mdr1a protein was produced. A cell lysate was prepared from the proximal colon of Mdr1a knock-out rat. Control cell lysate was prepared from a human neuroblastoma cell line. As shown on FIG. 36, no Mdr1a protein was detected in the Mdr1a (−/−) animal, indicating that the Mdr1a locus was inactivated.

### Example 68

### Identification of ZFNs that Edit the Mdr1b Locus

ZFNs that target and cleave the Mdr1b gene were identified essentially as described above. The rat Mdr1b gene (NM_{—}012623) was scanned for putative zinc finger binding sites. ZFNs were assembled and tested essentially as described in Example 64. This assay revealed that the ZFN pair targeted to bind 5′-agGAGGGGAAGCAGGGTtccgtggatga-3′ (SEQ ID NO: 166; contact sites in uppercase) and 5′-atGCTGGTGTTCGGatacatgacagata-3′ (SEQ ID NO: 167) cleaved within the Mdr1b locus.

### Example 69

### Identification of ZFNs that Edit the Mrp1 Locus

ZFNs that target and cleave the Mrp1 gene were identified essentially as described above in Example 64. The rat Mrp1 gene (NM_{—}022281) was scanned for putative zinc finger binding sites. ZFNs were assembled and tested essentially as described in Example 64. This assay revealed that the ZFN pair targeted to bind 5′-gaAGGGCCCAGGTTCTAagaaaaagcca-3′ (SEQ ID NO: 168; contact sites in uppercase) and 5′-tgCTGGCTGGGGTGGCTgttatgatcct-′3′ (SEQ ID NO: 169) cleaved within the Mrp1 locus.

### Example 70

### Editing the Mrp1 Locus

Rat embryos were microinjected with mRNA encoding the active pair of Mrp1 ZFNs essentially as described in Example 65. The injected embryos were incubated and DNA was extracted from the resultant animals. The targeted region of the Mrp1 locus was PCR amplified using appropriate primers. The amplified DNA was subcloned into a suitable vector and sequenced using standard methods. FIG. 37 presents DNA sequences of edited Mrp1 loci in two animals. One animal had a 43 bp deletion in exon 11, and a second animal had a 14 bp deletion in exon 11. These deletions disrupt the reading frame of the Mrp1 coding region.

### Example 71

### Identification of ZFNs that Edit the Mrp2 Locus

ZFNs that target and cleave the Mrp2 gene were identified essentially as described above in Example 64. The rat Mrp2 gene (NM_{—}012833) was scanned for putative zinc finger binding sites. ZFNs were assembled and tested essentially as described in Example 64. This assay revealed that the ZFN pair targeted to bind 5′-ttGCTGGTGACtGACCTTgttttaaacc-3′ (SEQ ID NO: 170; contact sites in uppercase) and 5′-ttGAGGCGGCCATGACAAAGgacctgca-′3′ (SEQ ID NO: 171) cleaved within the Mrp2 locus.

### Example 72

### Editing the Mrp2 Locus

Rat embryos were microinjected with mRNA encoding the active pair of Mrp2 ZFNs essentially as described in Example 65. The injected embryos were incubated and DNA was extracted from the resultant animals. The targeted region of the Mrp2 locus was PCR amplified using appropriate primers. The amplified DNA was subcloned into a suitable vector and sequenced using standard methods. FIG. 38 presents DNA sequence of an edited Mrp2 locus in which 726 bp was deleted from exon 7, thereby disrupting the reading frame of the Mrp2 coding region.

### Example 73

### Identification of ZFNs that Edit the BCRP Locus

ZFNs that target and cleave the BCRP gene were identified essentially as described above in Example 64. The rat BCRP gene (NM_{—}181381) was scanned for putative zinc finger binding sites. ZFNs were assembled and tested essentially as described in Example 64. This assay revealed that the ZFN pair targeted to bind 5′-atGACGTCAAGGAAGAAgtctgcagggt-3′ (SEQ ID NO: 172; contact sites in uppercase) and 5′-acGGAGATTCTTCGGCTgtaatgttaaa-′3′ (SEQ ID NO: 173) cleaved within the BCRP locus.

### Example 74

### Editing the BCRP Locus

Rat embryos were microinjected with mRNA encoding the active pair of BCRP ZFNs essentially as described in Example 65. The injected embryos were incubated and DNA was extracted from the resultant animals. The targeted region of the BCRP gene was PCR amplified using appropriate primers. The amplified DNA was subcloned into a suitable vector and sequenced using standard methods. FIG. 39 presents the DNA sequences of edited BCRP loci in two founder animals. One animal had a 588 bp deletion in exon 7, and the second animal had a 696 bp deletion in exon 7. These deletions disrupt the reading frame of the BCRP coding region.

### Example 75

### Disruption of Mdr1a

In vitro preparation of ZFN mRNAs: the ZFN expression plasmids were obtained from Sigma's CompoZr product line. Each plasmid was linearized at the XbaI site, which is located at the 3′ end of the FokI ORF. 5′ capped and 3′ polyA tailed message RNA was prepared using either MessageMax T7 Capped transcription kit and poly (A) polymerase tailing kit (Epicentre Biotechnology, Madison, Wis.) or mMessage Machine T7 kit and poly (A) tailing kit (Ambion, Austin, Tex.). The poly A tailing reaction was precipitated twice with an equal volume of 5 M NH4OAc and then dissolved in injection buffer (1 mM Tris-HCl, pH 7.4, 0.25 mM EDTA). mRNA concentration was estimated using a NanoDrop 2000 Spectrometer (Thermo Scientific, Wilmington, Del.).

ZFN validation in cultured cells: In short, when ZFNs make a double-strand break at the target site that is repaired by the non-homologous end-joining pathway, deletions or insertions are introduced. The wild-type and mutated alleles are amplified in the same PCR reaction. When the mixture is denatured and allowed to re-anneal, the wild-type and mutated alleles form double strands with unpaired region around the cleavage site, which can be recognized and cleaved by a single strand specific endonuclease to generate two smaller molecules in addition to the parental PCR product. The presence of the cleaved PCR bands indicates ZFN activity in the transfected cells.

The NIH 3T3 cells were grown in DMEM with 10% FBS and antibiotics at 37° C. with 5% CO2. ZFN mRNAs were paired at 1:1 ratio and transfected into the NIH 3T3 cells to confirm ZFN activity using a Nucleofector (Lonza, Basel, Switzerland), following the manufacture's 96-well shuttle protocol for 3T3 cells. Twenty-four hours after transfection, culturing medium was removed, and cells were incubated with 15 ul of trypsin per well for 5 min at 37° C. Cell suspension was then transferred to 100 ul of QuickExtract (Epicentre) and incubated at 68° C. for 10 min and 98° C. for 3 min. The extracted DNA was then used as template in a PCR reaction to amplify around the target site with following primer pairs:

Mdr1a Cel-I F:
(SEQ ID NO: 174)
ctgtttcttgacaaaacaacactaggctc
Mdr1a Cel-I R:
(SEQ ID NO: 175)
gggtcatgggaaagagtttaaaatc

Each 50 ul PCR reaction contained 1 ul of template, 5 ul of buffer II, 5 ul of 10 uM each primer, 0.5 ul of AccuPrime High Fidelity (Invitrogen, Carsbad, Calif.) and 38.5 ul of water. The following PCR program was used: 95° C., 5 min, 35 cycles of 95° C., 30 sec, 60° C., 30 sec, and 68° C., 45 sec, and then 68° C., 5 min, 4° C. Three microliter of the above PCR reaction was mixed with 7 ul of 1× buffer II and incubated under the following program: 95° C., 10 min, 95° C. to 85° C., at −2° C./s, 85° C. to 25° C. at −0.1° C./s, 4° C. forever One microliter each of nuclease S and enhancer (Transgenomic, Omaha, Nebr.) were added to digest the above reaction at 42° C. for 20 min. The mixture is resolved on a 10% polyacrylamide TBE gel (Bio-Rad, Hercules, Calif.).

Microinjection and mouse husbandry: FVB/NTac and C57BL/6NTac mice were housed in static cages and maintained on a 14 h/10 h light/dark cycle with ad libitum access to food and water. Three to four week-old females were injected with PMS (5 I.U./per mouse) 48 h before hCG (5 I.U./mouse) injection. One-cell fertilized eggs were harvested 10-12 h after hCG injection for microinjection. ZFN mRNA was injected at 2 ng/ul. Injected eggs were transferred to pseudopregnant females (Swiss Webster (SW) females from Taconic Labs mated with vasectomized SW males) at 0.5 dpc.

Founder identification using mutation detection assay: toe clips were incubated in 100-200 ul of QuickExtract (Epicentre Biotechnology) at 50° C. for 30 min, 65° C. for 10 min and 98° C. for 3 min. PCR and mutation detection assay were done under the same conditions as in ZFN validation in cultured cells using the same sets of primers.

TA cloning and sequencing: to identify the modifications in founders, the extracted DNA was amplified with Sigma's JumpStart Taq ReadyMix PCR kit. Each PCR reaction contained 25 ul of 2× ReadyMix, 5 ul of primers, 1 ul of template, and 19 ul of water. The same PCR program was used as in ZFN validation in cultured cells. Each PCR reaction was cloned using TOPO TA cloning kit (Invitrogen) following the manufacture's instructions. At least 8 colonies were picked from each transformation, PCR amplified with T3 and T7 primers, and sequenced with either T3 or T7 primer. Sequencing was done at Elim Biopharmaceuticals (Hayward, Calif.).

PCR for detecting large deletions: to detect larger deletions, another set of primers were used for each of the target:

(SEQ ID NO: 176)
Mdr1a 800F: catgctgtgaagcagatacc
(SEQ ID NO: 177)
Mdr1a 800R: ctgaaaactgaatgagacatttgc

Each 50 ul PCR contained: 1 ul of template, 5 ul of 10× buffer 11, 5 ul of 10 uM of each 800F/R primer, 0.5 ul of AccuPrime Taq Polymerase High Fidelity (Invitrogen), and 38.5 ul of water. The following program was used: 95° C., 5 min, 35 cycles of 95° C., 30 sec, 62° C., 30 sec, and 68° C., 45 sec, and then 68° C., 5 min, 4° C., forever. The samples were resolved on a 1% agarose gel. Distinct bands with lower molecular weight than the wt were sequenced.

RNA preparation from tissues and RT-PCR: Mdr1a−/− or Mdr1a+/+ littermates were sacrificed for tissue harvest at 5-9 weeks of age. Large intestine, kidney and liver tissues were dissected and immediately used or archived for later processing, tissue biopsies were placed in RNAlater solution (Ambion) and stored at −20° C. Total RNA was prepared using GenElute Mammalian Total RNA Miniprep kit (Sigma) following manufacture's instructions. To eliminate any DNA contamination the RNA was treated with DNAseI (New England Biolabs, Ipswich, Mass.) before being loaded onto the purification columns. RT-PCR reaction was carried out with 1 ul of total RNA, primers RT-F (5′-GCCGATAAAAGAGCCATGTTTG) (SEQ ID NO: 178) and RT-R (5′-GATAAGGAGAAAAGCTGCACC) (SEQ ID NO: 179), using SuperScript™ III One-Step RT-PCR System with Platinum® Taq High Fidelity kit (Invitrogen). Reverse transcription and subsequent PCR were carried out with 1 cycle of 55° C. for 30 min. and 94° C. for 2 min. for cDNA synthesis; and 40 cycles of 94° C. for 15 sec, 56° C. for 30 sec, and 68° C. for 1 min for amplification. The PCR product was loaded in a 1.2% agarose gel and visualized with ethidium bromide.

TABLE 10
Summary of deletions in Mdr1a
−10 −5 −2 +2 +5 +10
GCCATCAGCCCTGTTICTTGGACTGTCAGCTGGT

| | Deletion size | Position |
|---|---|---|
| ID | (bp) + insertion | |
| 2 | 6 + A | −4, +2 |
| 3 | 4 + C | −1, +3 |
| 4 | 3 | −2, +1 |
| 5 | 646 | −640, +6 |
| 6 | 695 | −583, +112 |
| 7 | 19 | −14, +5 |
| 8 | 248 | −238, +10 |
| 11 | 417, 19 | (−528- −112),(−14, +5) |
| | 533 | −27, +506 |
| 13 | 392 | −20, +372 |
| 17 | 2 | −1, +1 |
| | 19 | −14, +5 |
| | 19 | −18, +1 |
| 18 | 2 | +301-+2 |
| 19 | 25 | −25- −−1 |
| 20 | 19 | −15, +6 |
| 21 | 533 | −524, +9 |
| | 584 | -579, +5 |
| 23 | 396 | −389, +7 |
| 25 | 533 | −6, +527 |
| 26 | 13 | −5, +8 |
| | 534 | −516, +18 |
| 27 | 75 | −72, +3 |
| | 19 | −14, +5 |
| | 7 | −2, +5 |
| 28 | 731 | −724, +7 |
| 29 | 314 | −306, +8 |
| | 319 | −306, +13 |
| | 22 | −7, +15 |
| 31 | 11 | −4, +7 |
| 32 | 23 | −9, +14 |
| | 13 | −6, +7 |
| | 9 | −8, +1 |
| 34 | 6 | −2, +4 |
| 36 | 19 | −14, +5 |
| 38 | 430 | −423, +7 |
| | 28 | −25, +3 |

Interestingly, three small deletions were each found in two founders: a 19 bp deletion in founders 7 and 36, a 21 bp deletion in founders 17 and 27, and a 6 bp deletion in founders 34 and 44 (FIG. 43).

A high rate of germline transmission from Mdr1a founders was observed. Nine of the founders were chosen to backcross to the wild-type FVB/N mice to the F1 generation, all of which transmitted at least one mutant allele to their offspring. Seven founders transmitted multiple mutated alleles. Interestingly, in some cases, novel alleles that were not identified in founders also transmitted germline, such as founders 6, 8, 13, 21, and 44 (Table 11).

TABLE 11
Alleles transmitted in germline

| | Deletion | # | | | % |
|---|---|---|---|---|---|
| Founder ID | (bp) | Hets | Wildtype | Total | Transmission |
| 6 | Small | 5 | 2 | 9 | 77.8 |
| | 694 | 2 | | | |
| 8 | Small | 3 | 0 | 4 | 100.0 |
| | 248 | 1 | | | |
| 11 | 417, 19 | 3 | 3 | 7 | 57.1 |
| | 533 | 1 | | | |
| 13 | 2 | 1 | 0 | 1 | 100.0 |
| 21 | 533 + 5 bp | 4 | 2 | 12 | 58.3 |
| | 47 | 1 | | | |
| | 19 | 1 | | | |
| | 21 | 1 | | | |
| 23 | 396 | 14 | 15 | 29 | 48.3 |
| 26 | 534 | 2 | 0 | 15 | 100.0 |
| | 19 | 8 | | | |
| | 11 | 5 | | | |
| 27 | 75 | 4 | 17 | 37 | 54.1 |
| | 19 | 10 | | | |
| | 7 | 6 | | | |
| 44 | 455 | 1 | 6 | 16 | 56.3 |
| | 7 | 1 | | | |
| | 6 | 7 | | | |

To verify that deletion in the Mdr1a gene abolishes its expression, we performed RT-PCR on total RNA from liver, kidney and intestine of Mdr1a−/− mice established from founder 23, with a 396 bp deletion (FIG. 44A), using a forward and a reverse primer located in exons 5 and 9, respectively. The Mdr1a protein is differentially expressed in tissues. Liver and large intestine predominantly express Mdr1a, and kidney expresses both Mdr1a and Mdr1b. Samples from all the Mdr1a−/− tissues produced a smaller product at lower yield than corresponding wild-type samples, with a sequence correlating to exon 7 skipping, which introduces multiple premature stop codons in exon 8 in the mutant animals.

The RT-PCR results demonstrate that the Mdr1a−/− samples produce a transcript missing the 172 bp exon 7 at lower than wild-type level, possibly due to the premature stop codons introduced by exon skipping (FIG. 44B) that lead to non-sense mediated decay. In the Mdr1a−/− samples, there were faint bands at and above the size of the wild-type transcript, which are most likely PCR artifact because amplification of those bands excised from the gel yielded mostly the exon skipped product. The bands at the wild-type size in the second round of PCR were mixtures that did not yield readable sequences (not shown). The mouse Mdr1a gene has 28 exons, and the encoded protein is composed of two units of six transmembrane domains (TMs 1-6 and TMs 7-12) and an ATP binding site with a linker region in between. All 12 TM domains as well as the two ATP-binding motifs are essential for Mdr1a function. The Mdr1a ZFNs target exon 7, which encodes TMs 3 and 4. A partial protein resulting from exon skipping and premature translational terminations will not be functional. The Mdr1a−/− mice derived from founder 23 thus represent a functional knock-out.

To validate potential off-target sites of Mdr1a ZFN's, we identified 20 sites in the mouse genome that are most similar to the Mdr1a target site, all with 5 bp mismatches from the ZFN binding sequence. One site is in the Mdr1b gene, which is 88% identical to the Mdr1a gene. To validate the specificity of the Mdr1a ZFNs, we tested the Mdr1b site in all 44 Mdr1a F0 pups using mutation detection assay. None of the 44 pups had an NHEJ event at the Mdr1b site (FIG. 45). The finding that no modifications were detected at the Mdr1b site in any of the 44 live births indicates specificity of the Mdr1a ZFNs. In addition, undesired modifications at loci unlinked to the target site will be lost during subsequent breeding.

Table 12 lists sites among twenty sites in the mouse genome that were checked for off-target activity of Mdr1a ZFNs, which are most similar (with five mismatches) to the Mdr1a target site. Listed are the numbers of the chromosomes they are on and gene names if known. All the mismatched bases are in lower case. The spacer sequence between the binding sites is in bold letters.

TABLE 12
Potential off-target sites for Mdr1a ZFNs

| Chr. | Target | | SEQ ID |
|---|---|---|---|
| No. | Name | Binding Sequence | NO: |
| 5 | Abcb1a | GCCATCAGCCCTGTTCTTGGACTGTCAGCTGGT | 180 |
| 1 | Pld5 | GCCATCAGCtCTCAAAGAGGACTGTaAGaaGcT | 181 |
| 2 | | GCCAaCAGCtCTATTTT-GGACTcTCcGCTGcT | 182 |
| 3 | Slc33a1 | GCCATCAGCtCTATAACAtGACTGTCtaCTGaT | 183 |
| 3 | Syt11 | GtCAcCAaCCCTCTCCATGGAaaGTCAGCTGGT | 184 |
| 4 | | GaCtTCAGCCCTGACTGCtGACTGgCAaCTGGT | 185 |
| 4 | Anp32b | GCCAgCAGCCCTTTCCTTGaAggGTCAGCTaGT | 186 |
| 5 | Pitpnm2 | GCCATCAGCCCgCTCATGaGcCTGTttGCTGGT | 187 |
| 5 | | GCCAgCAGCCCTGCCTG-GGcCTGgCAGtTaGT | 188 |
| 5 | Abcb1b | GCtgTCAGCCCTCTTATTGGAtTGTCAtCTGcT | 189 |
| 6 | Mitf | GCCcTCAGCCCTCGAGATGctCTGTCAtCaGGT | 190 |
| 7 | lqck | GCCATCAGCCCaCTGTG-GGACTtTgAGtgGGT | 191 |
| 8 | Kifc3 | caCcTgAGCCCgCAACT-GGACTGTCAGCTGGT | 192 |
| 8 | | cCCATCAaCaCTAACACAGGACTGgCAtCTGGT | 193 |
| 10 | Oprm1 | tCCAgCAGCtCTGTCTG-GGACTGTtAGaTGGT | 194 |
| 10 | Pcbp3 | cCCAaCAGCCCTATTAG-GGACaGgCAcCTGGT | 195 |
| 11 | | GCCATCAGgCaTGGAGA-GGACatTCAGCTGGa | 196 |
| 12 | | GCCATCgcCCCTGGCCT-GGAtgGTCtGCTGGT | 197 |
| 12 | | cCCATCAGCaCTGTGGACGGtCgGTCAtCTGGT | 198 |
| 15 | | GCCAggAGCCtTTCAAGTGGACTGTCAGtTGcT | 199 |
| 16 | EtvS | GCCAgCAGCtgTGACTGTGGgCTaTCAGCTGGT | 200 |

Table 13 below presents the amino acid sequences of helices of the active ZFNs.

TABLE 13
Amino acid sequences of helices of active ZFNs

| Name | Sequence of Zinc Finger Helices | SEQ ID NO: |
|---|---|---|
| Mdr1a | DRSHLSR TSGNLTR QSSDLSR RSDHLTQ | 201 |
| Mdra | TSGHLSR QSSDLSR QSADRTK RSDVLSE | 202 |
| | QSGHLSR | |
| Mdr1b | TSGHLSR RSDNLSE RNANRIT RSDHLSE | 203 |
| | RNDNRKR | |
| Mdr1b | RSDHLSE NNSSRTR TSGHLSR QSSDLRR | 204 |
| MRP1 | TNGQLKE TSSSLSR RSDNLSE ASKTRKN | 205 |
| | RSDHLTQ | |
| MRP1 | DRSALSR RSDALAR RSDHLSR QSSDLRR | 206 |
| | RSDVLSE | |
| MRP2 | TSDHLTE DRSNLSR DRSNLTR TSGHLSR | 207 |
| | QSSDLRR | |
| MRP2 | RSDNLSV QNATRIN RSDALST DRSTRTK | 208 |
| | RSDDLSR RNDNRTK | |
| BCRP | QSGNLAR QSGNLAR RSDSLST DNASRIR | 209 |
| | DRSNLTR | |
| BCRP | QSSDLSR RNDDRKK RREDLIT TSSNLSR | 210 |
| | QSGHLSR | |

### Example 76

### Genome Editing of APP Locus

Zinc finger nucleases (ZFNs) that target and cleave the APP locus of rats were designed, assembled, and validated using strategies and procedures previously described (see Geurts et al. Science (2009) 325:433). ZFN design made use of an archive of pre-validated 1-finger and 2-finger modules. The rat APP gene region was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay identified a pair of active ZFNs that edited the APP locus. The zinc finger binding sites were 5′-GCCAGCACCCCTGACgcag-3′ (SEQ ID NO:213) and 5′-tcGACAAGTACCTGGAG-3′ (SEQ ID NO:214).

To mediate editing of the APP gene locus in animals, fertilized rat embryos were microinjected with mRNA encoding the active pair of ZFNs using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the APP locus was PCR amplified using appropriate primers. The amplified DNA was subcloned into a suitable vector and sequenced using standard methods. FIG. 32 presents edited APP loci in two founder animals; one had a 292 bp deletion in exon 9 (FIG. 32A) and the other had a 309 bp deletion in exon 9 (FIG. 32B).

### Example 77

### Genome Editing of ApoE Locus

ZFNs with activity at the ApoE locus were identified as described above. That is, the rat ApoE gene (NM_{—}138828) was scanned for putative zinc finger binding sites, and pairs of ZFNs were assembled and tested essentially as described in Example 76. It was found that the ZFN pair targeted to bind 5′-aaGCGGTTCAGGGCCTGctcccagggtt-3′ (SEQ ID NO:215; contact sites in upper case) and 5′-ggGATTACCTGcGCTGGGtgcagacgct-3′ (SEQ ID NO:216) cleaved the ApoE locus.

Fertilized one-cell embryos were injected with mRNAs encoding the active ZFN pair as described above in Example 76. The resultant animals were analyzed as detailed in Example 76. FIG. 30 presents two edited ApoE loci. One animal had a 16 bp deletion in the target sequence of exon 2, and a second animal had a 1 bp deletion in the target sequence of exon 2. These deletions disrupt the reading frame of the ApoE coding region.

### Example 78

### Genome Editing of BDNF Locus

To identify ZFNs that target and cleave the BDNF locus, the rat BDNF gene (NM_{—}012513) was scanned for putative zinc finger binding sites. The ZFNs pairs were assembled and tested essentially as described in Example 76. This analysis revealed that the ZFN pair targeted to bind 5′-cgGGGTCGGAGtGGCGCCgaaccctcat-3′ (SEQ ID NO:217) and 5′-cgGGGTCGGAGtGGCGCCgaaccctcat-3′ (SEQ ID NO:218) edited the BDNF locus.

Fertilized rat embryos were microinjected with mRNAs encoding the active ZNF pair and analyzed essentially as described above in Example 76. FIG. 46 presents edited BDNF loci in two founder animals; one had a 14 bp deletion in the target sequence in exon 2 and the other had a 7 bp deletion in the target sequence in exon 2.

The genetically modified rats were observed for phenotypic changes. Homozygous animals died within 2 weeks of birth. Heterozygous and homozygous animals were smaller in size than corresponding control animals (i.e., derived from embryos microinjected with GFP mRNA).

### Example 79

### Genome Editing of PSEN1 in a Model Organism

ZFN-mediated genome editing may be used to study the effects of a “knock-out” mutation in an AD-related chromosomal sequence, such as a chromosomal sequence encoding the PSEN1 protein, in a genetically modified model animal and cells derived from the animal. Such a model animal may be a rat. In general, ZFNs that bind to the rat chromosomal sequence encoding the PSEN1 protein associated with AD may be used to introduce a deletion or insertion such that the coding region of the PSEN1 gene is disrupted such that a functional PSEN1 protein may not be produced.

Suitable fertilized embryos may be microinjected with capped, polyadenylated mRNA encoding the ZFN essentially as detailed above in Example 76. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the PSEN1 “knock-out” may be assessed in the genetically modified rat or progeny thereof. Furthermore, molecular analyses of AD-related pathways may be performed in cells derived from the genetically modified animal comprising a PSEN1 “knock-out”.

### Example 80

### Generation of a Humanized Rat Expressing a Mutant Form of Human PSEN2

Missense mutations in PSEN2, a part of the enzymatic complex that cleaves amyloid beta peptide from APP, cause type 4 familial AD. One such mutation is the M239V missense mutation where the methionine residue acid at position 239 in PSEN2 is replaced with a valine residue. ZFN-mediated genome editing may be used to generate a humanized rat wherein the rat PSEN2 gene is replaced with a mutant form of the human PSEN2 gene comprising the M239V mutation. Such a humanized rat may be used to study the development of the diseases associated with the mutant human PSEN2 protein. In addition, the humanized rat may be used to assess the efficacy of potential therapeutic agents targeted at the pathway leading to AD comprising PSEN2.

The genetically modified rat may be generated using the methods described in the Examples above. However, to generate the humanized rat, the ZFN mRNA may be co-injected with the human chromosomal sequence encoding the mutant PSEN2 protein into the rat embryo. The rat chromosomal sequence may then be replaced by the mutant human sequence by homologous recombination, and a humanized rat expressing a mutant form of the PSEN2 protein may be produced.

The table below presents the amino acid sequences of helices of the active ZFNs.

| | | SEQ ID |
|---|---|---|
| Name | Sequence of Zinc Finger Helices | NO: |
| ApoE | RSDALSV DSSHRTR RSDNLSE TSGSLTR RSDDLTR | 219 |
| ApoE | RSDHLSR QSSDLRR RSDVLSA DRSNRIK TSSNLSR | 220 |
| BDNF | DRSDLSR DRSHLAR RSHNLAR RSDDLSK | 221 |
| | RSAHLSR | |
| BDNF | RSDNLAR QSSDLRR RSSHLSR RSDALSR | 222 |
| | DRSDLSR | |

### Example 81

### Genome Editing of BZRAP1 in a Model Organism

Zinc finger nuclease (ZFN)-mediated genome editing may be used to study the effects of a “knock-out” mutation in an ASD-associated chromosomal sequence, such as a chromosomal sequence encoding the BZRAP1 protein, in a genetically modified model animal and cells derived from the animal. Such a model animal may be a rat. In general, ZFNs that bind to the rat chromosomal sequence encoding the BZRAP1 protein associated with ASD may be used to introduce a non-sense mutation into the coding region of the BZRAP1 gene, such that an active BZRAP1 protein may not be produced.

Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into rat embryos. The rat embryos may be at the single cell stage when microinjected. Control embryos may be injected with 0.1 mM EDTA. The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The development of the embryos following microinjection, and the development of ASD-related symptoms and disorders caused by the BZRAP1 “knock-out” may be assessed in the genetically modified rat. For BZRAP1, ASD-related symptoms and disorders may include development of rheumatoid arthritis and an altered inflammatory response against tumors. The results may be compared to the control rat injected with 0.1 mM EDTA, where the chromosomal region encoding the BZRAP1 protein is not altered. In addition, molecular analysis of ASD-related pathways may be performed in cells derived from the genetically modified animal comprising a BZRAP1 “knock-out”.

### Example 82

### Generation of a Humanized Rat Expressing a Mutant Form of Human Neurexin-1

Missense mutations in neurexin-1, a presynaptic protein that helps glue together neurons at the synapse, are associated with autism. One such mutation is the L18Q missense mutation where the leucine amino acid at position 18 in neurexin-1 is replaced with glutamine. ZFN-mediated genome editing may be used to generate a humanized rat wherein the rat NRXN1 gene is replaced with a mutant form of the human NRXN1 gene comprising the L18Q mutation. Such a humanized rat may be used to study the development of autism. In addition, the humanized rat may be used to assess the efficacy of potential autism therapeutic agents targeted at perforin-1.

The genetically modified rat may be generated using the methods described in Example 81 above. However, to generate the humanized rat, the ZFN mRNA may be co-injected with the human chromosomal sequence encoding the mutant neurexin-1 protein into the rat embryo. The rat chromosomal sequence may then be replaced by the mutant human sequence by homologous recombination, and a humanized rat expressing a mutant form of the neurexin-1 protein may be produced.

### Example 83

### Genome Editing of NOG Locus

Zinc finger nucleases (ZFNs) that target and cleave the NOG locus of rats may be designed, assembled and validated using strategies and procedures previously described (see Geurts et al. Science (2009) 325:433). ZFN design may make use of an archive of pre-validated 1-finger and 2-finger modules. The rat NOG gene region was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rat cells. Control cells may be injected with mRNA encoding GFP. Active ZFN pairs may be identified by detecting ZFN-induced double strand chromosomal breaks using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay may be used to identify a pair of active ZFNs that edited the APP locus.

To mediate editing of the NOG gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA may be isolated using standard procedures. The targeted region of the NOG locus may be PCR amplified using appropriate primers. The amplified DNA may be subcloned into a suitable vector and sequenced using standard methods.

### Example 84

### Genome Editing of BMP4 in a Model Organism

ZFN-mediated genome editing may be used to study the effects of a “knock-out” mutation in neurodevelopmental chromosomal sequence, such as a chromosomal sequence encoding the BMP4 protein, in a genetically modified model animal and cells derived from the animal. Such a model animal may be a rat. In general, ZFNs that bind to the rat chromosomal sequence encoding the BMP4 protein associated with a neurodevelopmental pathway may be used to introduce a deletion or insertion such that the coding region of the BMP4 gene is disrupted such that a functional BMP4 protein may not be produced.

Suitable fertilized embryos may be microinjected with capped, polyadenylated mRNA encoding the ZFN essentially as detailed above in Example 83. The frequency of ZFN-induced double strand chromosomal breaks may be determined using the Cel-1 nuclease assay, as detailed above. The sequence of the edited chromosomal sequence may be analyzed as described above. The development of the neurodevelopmental symptoms and disorders caused by the BMP4 “knock-out” may be assessed in the genetically modified rat or progeny thereof. Furthermore, molecular analyses of neurodevelopmental pathways may be performed in cells derived from the genetically modified animal comprising a BMP4 “knock-out”.

### Example 85

### Generation of a Humanized Rat Expressing a Mutant Form of Human BMP4

Four missense mutations in BMP4 were detected in a population of human spina bifida aperta patients. ZFN-mediated genome editing may be used to generate a humanized rat wherein the rat BMP4 gene is replaced with a mutant form of the human BMP4 gene associated with spina bifida aperta, or any combination of the four mutations. Such a humanized rat may be used to study the development of the spina bifida aperta associated with the mutant human BMP4 protein. In addition, the humanized rat may be used to assess the efficacy of potential therapeutic agents targeted at the pathway leading to spina bifida aperta comprising BMP4.

The genetically modified rat may be generated using the methods described in the Example 83. However, to generate the humanized rat, the ZFN mRNA may be co-injected with the human chromosomal sequence encoding the mutant BMP4 protein into the rat embryo. The rat chromosomal sequence may then be replaced by the mutant human sequence by homologous recombination, and a humanized rat expressing a mutant form of the BMP4 protein may be produced.

### Example 86

### Generation of a Humanized Rat Expressing a Mutant Form of Human Perforin-1

Missense mutations in perforin-1, a critical effector of lymphocyte cytotoxicity, lead to a spectrum of diseases, from familial hemophagocytic lymphohistiocytosis to an increased risk of tumorigenesis. One such mutation is the V50M missense mutation where the valine amino acid at position 50 in perforin-1 is replaced with methionine. ZFN-mediated genome editing may be used to generate a humanized rat wherein the rat PRF1 gene is replaced with a mutant form of the human PRF1 gene comprising the V50M mutation. Such a humanized rat may be used to study the development of the diseases associated with the mutant human perforin-1 protein. In addition, the humanized rat may be used to assess the efficacy of potential therapeutic agents targeted at the inflammatory pathway comprising perforin-1.

The genetically modified rat may be generated using the methods described in Example 38 above. However, to generate the humanized rat, the ZFN mRNA may be co-injected with the human chromosomal sequence encoding the mutant perforin-1 protein into the rat embryo. The rat chromosomal sequence may then be replaced by the mutant human sequence by homologous recombination, and a humanized rat expressing a mutant form of the perforin-1 protein may be produced.

The table below presents the amino acid sequences of helices of the active ZFNs.

| | | SEQ |
|---|---|---|
| | | ID |
| Name | Sequence of Zinc Finger Helices | NO: |
| ApoE | RSDALSV DSSHRTR RSDNLSE TSGSLTR RSDDLTR | 223 |
| ApoE | RSDHLSR QSSDLRR RSDVLSA DRSNRIK TSSNLSR | 224 |

### Example 87

### Genome Editing of TRPM5 Locus

Zinc finger nucleases (ZFNs) that target and cleave the TRPM5 locus of rats may be designed, assembled, and validated using strategies and procedures previously described (see Geurts et al. Science (2009) 325:433). ZFN design may make use of an archive of pre-validated 1-finger and 2-finger modules. The rat TRPM5 gene region was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rat cells. Control cells may be injected with mRNA encoding GFP. Active ZFN pairs may be identified by detecting ZFN-induced double strand chromosomal breaks using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay may be used to identify a pair of active ZFNs that edited the TRPM5 locus.

To mediate editing of the TRPM5 gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA may be isolated using standard procedures. The targeted region of the TRPM5 locus may be PCR amplified using appropriate primers. The amplified DNA may be subcloned into a suitable vector and sequenced using standard methods.

### Example 88

### Genome Editing of ERAL1 in a Model Organism

ZFN-mediated genome editing may be used to study the effects of a “knock-out” mutation in nociception-related chromosomal sequence, such as a chromosomal sequence encoding the ERAL1 protein, in a genetically modified model animal and cells derived from the animal. Such a model animal may be a rat. In general, ZFNs that bind to the rat chromosomal sequence encoding the ERAL1 protein associated with a nociception pathway may be used to introduce a deletion or insertion such that the coding region of the ERAL1 gene is disrupted such that a functional ERAL1 protein may not be produced.

Suitable fertilized embryos may be microinjected with capped, polyadenylated mRNA encoding the ZFN essentially as detailed above in Example 87. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the ERAL1 “knock-out” may be assessed in the genetically modified rat or progeny thereof. Furthermore, molecular analyses of nociception-related pathways may be performed in cells derived from the genetically modified animal comprising a ERAL1 “knock-out”.

### Example 89

### Generation of a Humanized Rat Expressing a Mutant Form of Human SCN9A

Missense mutations in SCN9A, a sodium ion channel that is expressed at high levels in nociceptive dorsal root ganglion (DRG) neurons, are associated with erythromelagia, an inherited disorder characterized by symmetrical burning pain of the feet, lower legs, and hands. Three mutations have been characterized in SCN9A: W897X, located in the P-loop of domain 2; I767X, located in the S2 segment of domain 2; and S459X, located in the linker region between domains 1 and 2, any one of which results in a truncated non-functional protein. ZFN-mediated genome editing may be used to generate a humanized rat wherein the rat SCN9A gene is replaced with a mutant form of the human SCN9A gene comprising the W897X mutation, the I767X mutation, the S459X mutation, or any combination of the three mutations. Such a humanized rat may be used to study the development of the erythromelagia associated with the mutant human SCN9A protein. In addition, the humanized rat may be used to assess the efficacy of potential therapeutic agents targeted at the pathway leading to erythromelagia comprising SCN9A.

The genetically modified rat may be generated using the methods described in Example 87 above. However, to generate the humanized rat, the ZFN mRNA may be co-injected with the human chromosomal sequence encoding the mutant SCN9A protein into the rat embryo. The rat chromosomal sequence may then be replaced by the mutant human sequence by homologous recombination, and a humanized rat expressing a mutant form of the SCN9A protein may be produced.

### Example 90

### Identification of ZFNs that Edit the DISC1 Locus

The DISC1 gene in rat was chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The DISC1 gene region (NM_{—}175596) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-taGTCCCGGCAGGCTATcctgggcggtg-3′ (SEQ ID NO: 226; contact sites in uppercase) and 5′-ccGTCACCAGGCGGGACtggctgatgcg-3′ (SEQ ID NO: 227) cleaved within the DISC1 locus.

### Example 91

### Editing the DISC1 Locus in Rat Embryos

Capped, polyadenylated mRNA encoding the active pair of ZFNs was microinjected into fertilized rat embryos using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the DISC1 locus was PCR amplified using appropriate primers. The amplified DNA was subcloned into a suitable vector and sequenced using standard methods. FIG. 47 presents an edited DISC1 locus in which 20 bp was deleted from the target sequence in exon 5. This deletion disrupts the reading frame of the DISC1 coding region.

### Example 92

### Identification of ZFNs that Edit the BDNF Locus

To identify ZFNs that target and cleave the BDNF locus, the rat BDNF gene (NM_{—}012513) was scanned for putative zinc finger binding sites. The ZFNs were assembled and tested essentially as described in Example 90. This analysis revealed that the ZFN pair targeted to bind 5′-cgGGGTCGGAGtGGCGCCgaaccctcat-3′ (SEQ ID NO: 228) and 5′-ccGCCGTGGGGaGCTGAGcgtgtgtgac-3′ (SEQ ID NO: 229) cleaved within the BDNF locus.

Fertilized rat embryos were microinjected with mRNAs encoding the active ZNF pair and analyzed essentially as described above in Example 91. FIG. 46 presents edited BDNF loci in two founder animals; one had a 14 bp deletion in the target sequence in exon 2 and the other had a 7 bp deletion in the target sequence in exon 2.

The genetically modified rats were observed for phenotypic changes. Homozygous animals died within 2 weeks of birth. Heterozygous and homozygous animals were smaller in size than corresponding control animals (i.e., derived from embryos microinjected with GFP mRNA).

### Example 93

### Genome Editing of ErbB4 in a Model Organism

ZFN-mediated genome editing may be used to study the effects of a “knock-out” mutation in a chromosomal sequence associated with schizophrenia, such as a chromosomal sequence encoding the ErbB4 protein, in a genetically modified model animal and cells derived from the animal. Such a model animal may be a rat. In general, ZFNs that bind to the rat chromosomal sequence encoding the ErbB4 protein associated with schizophrenia may be used to introduce a deletion or insertion such that the coding region of the ErbB4 gene is disrupted such that a functional ErbB4 protein may not be produced.

Suitable fertilized embryos may be microinjected with capped, polyadenylated mRNA encoding the ZFN. The frequency of ZFN-induced double strand chromosomal breaks may be determined using the Cel-1 nuclease assay, as detailed above. The sequence of the edited chromosomal sequence may be analyzed as described above. The development of schizophrenia symptoms and disorders caused by the ErbB4 “knock-out” may be assessed in the genetically modified rat or progeny thereof. Furthermore, molecular analyses of schizophrenia-related pathways may be performed in cells derived from the genetically modified animal comprising an ErbB4 “knock-out”.

### Example 94

### Generation of a Humanized Rat Expressing a Mutant Form of Human TPH1

To develop a “humanized” animal model for the evaluation of schizophrenia symptoms and treatments, a rat comprising a genome including the human mutant form of TPH1 may be created. The human mutant form may be A218C that is found within intron 7 of TPH1; A218C is thought to be highly associated with schizophrenia. ZFN-mediated genome editing may be used to generate a humanized rat wherein the rat gene is replaced with the A218C mutant form of human TPH1. Such a humanized rat may be used to study the development of schizophrenia associated with the mutant human protein encoded by the mutated TPH1. In addition, the humanized rat may be used to assess the efficacy of potential therapeutic agents targeted at the pathway associated with TPH1.

The genetically modified rat may be generated using the methods described in Example 91 above. However, to generate the humanized rat, the ZFN mRNA may be co-injected with the human chromosomal sequence encoding the mutant TPH1 protein into the rat embryo. The rat chromosomal sequence may then be replaced by the mutant human sequence by homologous recombination, and a humanized rat expressing a mutant form of the protein may be produced.

The table below presents the amino acid sequences of helices of the active ZFNs.

| | | SEQ |
|---|---|---|
| | | ID |
| Name | Sequence of Zinc Finger Helices | NO: |
| DISC1 | NSGNLDK DRSHLSR QSGDLTR RSDTLSQ DRSARTR | 230 |
| DISC1 | DRSNLSR RSDNLRE RSDHLSA DSSTRKT DRSSRKR | 231 |
| BDNF | DRSDLSR DRSHLAR RSHNLAR RSDDLSK RSAHLSR | 232 |
| BDNF | RSDNLAR QSSDLRR RSSHLSR RSDALSR DRSDLSR | 233 |

### Example 95

### Identification of ZFNs that Edit the p53 Locus

The p53 gene was chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The rat p53 gene region (NM_{—}030989) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were transfected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-atCTGGAGGAAGACtGGAGAAcaagagc-3′ (SEQ ID NO:234; contact sites shown in uppercase) and 5′-atATTCTGGTAAGGAGCCGGgcaagagg-3′ (SEQ ID NO:235) edited the p53 gene.

### Example 96

### Editing of the p53 Locus in Rat Embryos

Capped, polyadenylated mRNA encoding the active pair of ZFNs was microinjected into fertilized rat embryos using standard procedures (e.g., see Geurts et al. (2009) supra). Control embryos were microinjected with saline or mRNA encoding GFP. The injected embryos were transferred to pseudopregnant female rats to be carried to parturition. Toe/tail of clips of each live born animal was harvested for DNA extraction and analysis using a Cel-1 assay. As shown in FIG. 48, about 25% of the experimental animals had an edited p53 gene locus.

### Example 97

### Inactivation of the p53 Locus in Rat

To determine that the edited p53 locus was inactivated, Western analyses were performed to confirm that no p53 protein was produced. Cell lysates were prepared from the kidney and liver of a wildtype animal and a p53 knock-out animal. As shown on FIG. 49, both cytoplasmic and nuclear lysates of the p53 knock-out animal were devoid of p53 protein. The levels of actin protein were constant among the wildtype and mutant samples, however. Thus, the p53 edited rat was a p53 knock-out rat.

### Example 98

### Identification of ZFNs that Edit the BCRP Locus in Rat

ZFNs that target and cleave the BCRP gene were identified essentially as described above in Example 95. The rat BCRP gene (NM_{—}1811381) was scanned for putative zinc finger binding sites. ZFNs were assembled and tested essentially as described in Example 95. It was found that the ZFN pair targeted to bind 5′-atGACGTCAAGGAAGAAgtctgcagggt-3′ (SEQ ID NO:236) and 5′-acGGAGATTCTTCGGCTgtaatgttaaa-3′ (SEQ ID NO:237) edited the BCRP gene.

### Example 99

### Editing the BCRP Locus

Rat embryos were microinjected with mRNA encoding the active pair of BCRP ZFNs essentially as described in Examples 95 and 96. The injected embryos were incubated and DNA was extracted from the resultant animals. The targeted region of the BCRP gene was PCR amplified using appropriate primers. The amplified DNA was subcloned into a suitable vector and sequenced using standard methods. FIG. 39 presents edited BCRP loci in two founder animals. One animal had a 588 bp deletion in exon 7, and the second animal had a 696 bp deletion in exon 7. These deletions disrupt the reading frame of the BCRP coding region.

### Example 100

### Editing the Pten Locus

ZFNs that target and cleave the Pten locus in rats were designed and tested for activity essentially as described above in Example 95. An active pair of ZFNs was identified. The DNA binding sites were 5′-CCCCAGTTTGTGGTCtgcca-3′ (SEQ ID NO:238) and 5′-gcTAAAGGTGAAGATCTA-3′ (SEQ ID NO:239). Capped, polyadenylated mRNA encoding the active pair may be microinjected into rat embryos and the resultant embryos may be analyzed as described in Examples 95 and 96. Accordingly, the Pten locus may be edited to contain a deletion or an insertion such that the coding region is disrupted and no functional protein is made.

The table below presents the amino acid sequences of helices of the active ZFNs.

| | | SEQ |
|---|---|---|
| | | ID |
| Name | Sequence of Zinc Finger Helices | NO: |
| p53 | QSGNLAR QSGHLSR DRSALSR QSGNLAR | 240 |
| | RSDALSR RSDALTQ | |
| p53 | RSDHLSE TSSDRTK RSDHLSA QSGSLTR RSDVLSE | 241 |
| | HSNARKT | |
| BRCP | QSGNLAR QSGNLAR RSDSLST DNASRIR DRSNLTR | 242 |
| BRCP | QSSDLSR RNDDRKK RREDLIT TSSNLSR QSGHLSR | 243 |

### Example 101

### Genome Editing of HTT in a Model Organism

ZFN-mediated genome editing may be used to study the effects of a “knock-out” mutation in a trinucleotide repeat expansion-related chromosomal sequence, such as a chromosomal sequence encoding the HTT protein, in a genetically modified model animal and cells derived from the animal. Such a model animal may be a rat. In general, ZFNs that bind to the rat chromosomal sequence encoding the HTT protein associated with trinucleotide repeat expansion disorders may be used to introduce a deletion or insertion such that the coding region of the HTT gene is disrupted such that a functional HTT protein may not be produced.

Suitable fertilized embryos may be microinjected with capped, polyadenylated mRNA encoding the ZFN according to known molecular biology techniques. The frequency of ZFN-induced double strand chromosomal breaks may be determined using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The sequence of the edited chromosomal sequence may be analyzed. The development of trinucleotide repeat expansion disorders caused by the HTT “knock-out” may be assessed in the genetically modified rat or progeny thereof. Furthermore, molecular analyses of trinucleotide repeat expansion-related pathways may be performed in cells derived from the genetically modified animal comprising a HTT “knock-out”.

### Example 102

### Generation of a Humanized Rat Expressing a Mutant Form of Human Genes involved in Trinucleotide Repeat Expansion Disorders

Mutations in any of the chromosomal sequences involved in trinucleotide repeat expansion disorders may be used in the generation of a humanized rat expressing a mutant form of the gene. The genes can htt, ar, fxn, atxn1, atxn2, atxn3, atxn7, atxn10, dmpk, atn1, cbp, vldlr, and combinations thereof. ZFN-mediated genome editing may be used to generate a humanized rat wherein the rat gene is replaced with a mutant form of the human gene comprising the mutation. Such a humanized rat may be used to study the development of the diseases associated with the mutant human protein encoded by the gene of interest. In addition, the humanized rat may be used to assess the efficacy of potential therapeutic agents targeted at the pathway leading to a trinucleotide repeat expansion disorder comprising the gene of interest.

The genetically modified rat may be generated using the methods described in the Example above. However, to generate the humanized rat, the ZFN mRNA may be co-injected with the human chromosomal sequence encoding the mutant protein into the rat embryo. The rat chromosomal sequence may then be replaced by the mutant human sequence by homologous recombination, and a humanized rat expressing a mutant form of the protein may be produced.

### Example 103

### Genome Editing of 5-HTT in a Model Organism

ZFN-mediated genome editing may be used to study the effects of a “knock-out” mutation in a neurotransmission-related chromosomal sequence, such as a chromosomal sequence encoding the 5-HTT protein, in a genetically modified model animal and cells derived from the animal. Such a model animal may be a rat. In general, ZFNs that bind to the rat chromosomal sequence encoding the 5-HTT protein associated with neurotransmission-related disorders may be used to introduce a deletion or insertion such that the coding region of the 5-HTT gene is disrupted such that a functional 5-HTT protein may not be produced.

Suitable fertilized embryos may be microinjected with capped, polyadenylated mRNA encoding the ZFN. The frequency of ZFN-induced double strand chromosomal breaks may be determined using the Cel-1 nuclease assay, as detailed above. The sequence of the edited chromosomal sequence may be analyzed as described above. The development of neurotransmission symptoms and disorders caused by the 5-HTT “knock-out” may be assessed in the genetically modified rat or progeny thereof. Furthermore, molecular analyses of neurotransmission-related pathways may be performed in cells derived from the genetically modified animal comprising an ErbB4 “knock-out”.

### Example 104

### Generation of a Humanized Rat Expressing a Mutant Form of Human Genes Involved in Neurotransmission

Mutations in any of the chromosomal sequences involved in neurotransmission disorders may be used in the generation of a humanized rat expressing a mutant form of the gene. The genes can be 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1K4, COMT, MAO, DBH, TyrH, CB1, CB2, FAAH, MAGL and combinations thereof. ZFN-mediated genome editing may be used to generate a humanized rat wherein the rat gene is replaced with a mutant form of the human gene comprising the mutation. Such a humanized rat may be used to study the development of the diseases associated with the mutant human protein encoded by the gene of interest. In addition, the humanized rat may be used to assess the efficacy of potential therapeutic agents targeted at the pathway leading to a neurotransmission disorder comprising the gene of interest.

The genetically modified rat may be generated using the methods described in the Examples above. However, to generate the humanized rat, the ZFN mRNA may be co-injected with the human chromosomal sequence encoding the mutant protein into the rat embryo. The rat chromosomal sequence may then be replaced by the mutant human sequence by homologous recombination, and a humanized rat expressing a mutant form of the protein may be produced.

### Example 105

### Genome Editing of the APH-1 Locus

Zinc finger nucleases (ZFNs) that target and cleave the APH-1 locus of rats may be designed, assembled, and validated using strategies and procedures previously described (see Geurts et al. Science (2009) 325:433). ZFN design may make use of an archive of pre-validated 1-finger and 2-finger modules. The rat APH-1 gene region may be scanned for putative zinc finger binding sites to which existing modules could be fused to generate a pair of 4-, 5-, or 6-finger proteins that may bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rat cells. Control cells may then be injected with mRNA encoding GFP. Active ZFN pairs may be identified by detecting ZFN-induced double strand chromosomal breaks using the Cel-1 nuclease assay. This assay may detect alleles of the target locus that deviate from wild type as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch may be cleaved by the surveyor nuclease Cel-1, and the cleavage products may be resolved by gel electrophoresis. This assay may identify a pair of active ZFNs that edit the APH-1 locus.

To mediate editing of the APH-1 gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA may be isolated using standard procedures. The targeted region of the APH-1 locus may then be PCR amplified using appropriate primers. The amplified DNA may be subcloned into a suitable vector and sequenced using standard methods.

### Example 106

### Genome Editing of Secretase-Related Genes in Model Organism Cells

ZFN-mediated genome editing may be tested in the cells of a model organism such as a rat using a ZFN that binds to the chromosomal sequence of a secretase-related gene such as APH-1A, APH-1B, PSEN1, NCSTN, or PEN-2 ZFNs may be designed and tested essentially as described in Example 105. ZFNs targeted to a specific secretase-related gene may be used to introduce a deletion or insertion such that the coding region of the gene of interest is inactivated.

### Example 107

### Genome Editing of Secretase-Related Genes in Model Organisms

The embryos of a model organism such as a rat may be harvested using standard procedures and injected with capped, polyadenylated mRNA encoding ZFNs that target secretase-related genes, as detailed above in Example 105. Donor or exchange polynucleotides comprising sequences for integration or exchange may be co-injected with the ZFNs. The edited chromosomal regions in the resultant animals may be analyzed as described above. The modified animals may be phenotypically analyzed for changes in behavior, learning, etc. Moreover, the genetically modified animal may be used to assess the efficacy of potential therapeutic agents for the treatment of a secretase disorder.

### Example 108

### Genome Editing of the SOD1 Locus

Zinc finger nucleases (ZFNs) that target and cleave the SOD1 locus of rats may be designed, assembled, and validated using strategies and procedures previously described (see Geurts et al. Science (2009) 325:433). ZFN design may make use of an archive of pre-validated 1-finger and 2-finger modules. The rat SOD1 gene region may be scanned for putative zinc finger binding sites to which existing modules could be fused to generate a pair of 4-, 5-, or 6-finger proteins that would may a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rat cells. Control cells may then be injected with mRNA encoding GFP. Active ZFN pairs may be identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” may form at the site of mismatch that may be cleaved by the surveyor nuclease Cel-1, and the cleavage products may be resolved by gel electrophoresis. This assay may be used to identify a pair of active ZFNs that edited the SOD1 locus.

To mediate editing of the SOD1 gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA may be isolated using standard procedures. The targeted region of the SOD1 locus may be PCR amplified using appropriate primers. The amplified DNA may be subcloned into a suitable vector and sequenced using standard methods.

### Example 109

### Genome Editing of ALS-Related Genes in Model Organism Cells

ZFN-mediated genome editing may be tested in the cells of a model organism such as a rat using a ZFN that binds to the chromosomal sequence of a ALS-related gene such as SOD1, ALS2, FUS, TARDBP, or VEGF(A, B, or C) ZFNs may be designed and tested essentially as described in Example 108. ZFNs targeted to a specific ALS-related gene may be used to introduce a deletion or insertion such that the coding region of the gene of interest is inactivated.

### Example 110

### Genome Editing of ALS-Related Genes in Model Organisms

The embryos of a model organism such as a rat may be harvested using standard procedures and injected with capped, polyadenylated mRNA encoding ZFNs that target ALS-related genes, as detailed above in Example 108. Donor or exchange polynucleotides comprising sequences for integration or exchange may be co-injected with the ZFNs. The edited chromosomal regions in the resultant animals may be analyzed as described above. The modified animals may be phenotypically analyzed for changes in behavior, learning, etc. Moreover, the genetically modified animal may be used to assess the efficacy of potential therapeutic agents for the treatment of ALS.

### Example 111

### Genome Editing of the prnd Locus

Zinc finger nucleases (ZFNs) that target and cleave the prdn locus of rats may be designed, assembled, and validated using strategies and procedures previously described (see Geurts et al. Science (2009) 325:433). ZFN design made use of an archive of pre-validated 1-finger and 2-finger modules. The rat prdn gene region may be scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rat cells. Control cells may be injected with mRNA encoding GFP. Active ZFN pairs may be identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch will be cleaved by the surveyor nuclease Cel-1, and the cleavage products can be resolved by gel electrophoresis. This assay may identify a pair of active ZFNs that edited the prnd locus.

To mediate editing of the prnd gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA can be isolated using standard procedures. The targeted region of the prnd locus is to be PCR amplified using appropriate primers. The amplified DNA is to be subcloned into a suitable vector and sequenced using standard methods.

### Example 112

### Genome Editing of Dpl Genes in Model Organism Cells

ZFN-mediated genome editing may be used to study the effects of a “knock-out” mutation in an AD-related chromosomal sequence, such as a chromosomal sequence encoding the Dpl protein, in a genetically modified model animal and cells derived from the animal. Such a model animal may be a rat. In general, ZFNs that bind to the rat chromosomal sequence encoding the Dpl protein associated with AD may be used to introduce a deletion or insertion such that the coding region of the Dpl gene (Prnd) is disrupted such that a functional Dpl protein may not be produced.

Suitable fertilized embryos may be microinjected with capped, polyadenylated mRNA encoding the ZFN essentially as detailed above in Example 111. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the Dpl “knock-out” may be assessed in the genetically modified rat or progeny thereof. Furthermore, molecular analyses of AD-related pathways may be performed in cells derived from the genetically modified animal comprising a Dpl “knock-out”.

### Example 113

### Genome Editing of Prp Genes in Model Organisms

Coding polymorphism at PrP codon 129 has a strong association with disease susceptibility and phenotype modifying effect, especially when the amino acid at codon 129 is methionine or valine. ZFN-mediated genome editing may be used to generate a humanized rat wherein the rat Prp gene is replaced with a mutant form of the human Prpn gene comprising sequence with 129M or 129V. Such a humanized rat may be used to study the development of the diseases associated with the mutant human PSEN2 protein. In addition, the humanized rat may be used to assess the efficacy of potential therapeutic agents targeted at the pathway leading to prion disorder comprising neurotoxic PrP isoform.

### Example 114

### Genome Editing of Agouti in Model Organism Cells

Zinc finger nuclease (ZFN)-mediated genome editing may be tested in the cells of a model organism such as an equine using a ZFN that binds to the chromosomal sequence of a hair color-related gene of the equine cell such as MSH receptor proteins, agouti signaling protein (ASIP) and melanophilin (MLPH). The particular coat color-related gene to be edited may be a gene having identical DNA binding sites to the DNA binding sites of the corresponding equine homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into equine cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch may be cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected hair color-related gene locus in equine cells using a ZFN.

### Example 115

### Genome Editing of Agouti in Model Organism Embryos

The embryos of a model organism such as an equine may be harvested using standard procedures and injected with capped, polyadenylated mRNA encoding a ZFN similar to that described in Example 114. The equine embryos may be generally at the one-cell stage when microinjected. Control embryos may be injected with 0.1 mM EDTA. The frequency of ZFN-induced double strand chromosomal breaks may be estimated using the Cel-1 assay as described in Example 114. The cutting efficiency may be estimated using the CEl-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

### Example 116

### Generation of a Humanized Equine Expressing a Mutant Form of Human SCID

The first human mutation in the gene encoding DNA-PKcs (DNA-dependent protein kinase catalytic subunit) has been identified in a radiosensitive T-B-SCID patient. A mutation in the DNA-PKcs gene has been predicted for a long time, but spontaneous mutations had only been identified in mouse, horse and dog models. A single base change at DNA-PKcs may lead to alteration of a disease-associated kinase subunit protein. ZFN-mediated genome editing may be used to generate a humanized equine wherein the equine DNA-PKcs is replaced with a mutant form of the human DNA-PKcs comprising one or more mutations. Such a humanized equine may be used to study the development of the diseases associated with the mutant human DNA-PKcs protein. In addition, the humanized equine may be used to assess the efficacy of potential therapeutic agents targeted at the pathway leading to immunodeficiency comprising DNA-PKcs.

The genetically modified equine may be generated using the methods described in the Examples above. However, to generate the humanized equine, the ZFN mRNA may be co-injected with the human chromosomal sequence encoding the mutant DNA-PKcs protein into the equine embryo. The equine chromosomal sequence may then be replaced by the mutant human sequence by homologous recombination, and a humanized equine expressing a mutant form of the DNA-PKcs protein may be produced.

1. A method for editing a chromosomal sequence, the method comprising:
a. introducing into a non-human embryo comprising the chromosomal sequence at least one nucleic acid encoding a zinc finger nuclease, the embryo being other than a zebrafish or fruit fly embryo, the zinc finger nuclease being able to bind a target sequence in the chromosomal sequence and to cleave a cleavage site in the chromosomal sequence, and, optionally,
(i) at least one donor polynucleotide comprising a donor sequence for integration, an upstream sequence, and a downstream sequence, wherein the donor sequence is flanked by the upstream sequence and the downstream sequence, and wherein the upstream sequence and the downstream sequence share substantial sequence identity with either side of the cleavage site, or
(ii) at least one exchange polynucleotide comprising an exchange sequence that is substantially identical to a portion of the chromosomal sequence at the cleavage site and further comprising at least one nucleotide change; and
b. culturing the embryo to allow expression of the zinc finger nuclease such that the zinc finger nuclease introduces a double-stranded break into the chromosomal sequence at the cleavage site, and wherein the double-stranded break is repaired by either
(i) a non-homologous end-joining repair process such that a mutation is introduced into the chromosomal sequence, or
(ii) a homology-directed repair process such that the donor sequence is integrated into the chromosomal sequence or the exchange sequence is exchanged with the portion of the chromosomal sequence.

2. The method of claim 1, wherein the embryo is a mammalian embryo.

3. The method of claim 1, wherein the embryo is a one cell embryo.

4. The method of claim 1, wherein more than one nucleic acid encoding a zinc finger nuclease is introduced into the embryo.

5. The method of claim 1, wherein the nucleic acid encoding a zinc finger nuclease is an RNA.

6. The method of claim 5, wherein the RNA is capped.

7. The method of claim 5, wherein the RNA is polyadenylated.

8. The method of claim 1, wherein more than one of a polynucleotide chosen from the donor polynucleotides, the exchange polynucleotides, or any combination thereof are introduced into the embryo.

9-10. (canceled)

11. A non-human animal comprising at least one edited chromosomal sequence, the animal being created by the method of claim 1.

12. The non-human animal of claim 11, wherein the animal is a rodent.

13. The non-human animal of claim 11, wherein the animal is a livestock animal.

14. The non-human animal of claim 11, wherein the animal is a companion animal.

15. A cell comprising at least one edited chromosomal sequence, the cell being derived from an animal created using the method of claim 1.

16-17. (canceled)

18. The cell of claim 15, wherein the cell is a cultured cell, a primary cell, or a stem cell.

19. A non-human embryo, the embryo being other than a zebrafish or fruit fly embryo and comprising at least one nucleic acid encoding a zinc finger nuclease that is able to bind a target sequence in the chromosomal sequence and cleave a cleavage site in the chromosomal sequence, and, optionally,
(i) at least one donor polynucleotide comprising a donor sequence for integration, an upstream sequence, and a downstream sequence, wherein the donor sequence is flanked by the upstream sequence and the downstream sequence, and wherein the upstream sequence and the downstream sequence share substantial sequence identity with either side of the cleavage site, or
(ii) at least one exchange polynucleotide comprising an exchange sequence that is substantially identical to a portion of the chromosomal sequence at the cleavage site and which further comprises at least one nucleotide change.

20. The embryo of claim 19, wherein the embryo is a one cell embryo.

21. The embryo of claim 19, wherein the embryo is a mammalian embryo.
